SEQUENCE LISTING (1662 sequences)

SEQ ID NO: 1 - DNA, 36 bp, Artificial Sequence
  source: /note="Description of Artificial Sequence: Syntheticoligonucleotide"
accgatcatgtctgggttaccagttagtcgtgtctc

SEQ ID NO: 2 - DNA, 36 bp, Artificial Sequence
  source: /note="Description of Artificial Sequence: Syntheticoligonucleotide"
tcgtcttgaaccaatgaccaaatgcaagccctccat

SEQ ID NO: 3 - DNA, 36 bp, Artificial Sequence
  source: /note="Description of Artificial Sequence: Syntheticoligonucleotide"
atacgagggcacaggagctattagtgtagcgaaagg

SEQ ID NO: 4 - DNA, 36 bp, Artificial Sequence
  source: /note="Description of Artificial Sequence: Syntheticoligonucleotide"
tcaccacctgatttctgttgccaccgcatcagttta

SEQ ID NO: 5 - DNA, 36 bp, Artificial Sequence
  source: /note="Description of Artificial Sequence: Syntheticoligonucleotide"
tttccatacctgcagcggcatctattcatgacatgt

SEQ ID NO: 6 - DNA, 36 bp, Artificial Sequence
  source: /note="Description of Artificial Sequence: Syntheticoligonucleotide"
ccattaagctcacccacagggagttggagtctaaac

SEQ ID NO: 7 - DNA, 36 bp, Artificial Sequence
  source: /note="Description of Artificial Sequence: Syntheticoligonucleotide"
gttagaaggaccacaacggaccagagagtgcatata

SEQ ID NO: 8 - DNA, 36 bp, Artificial Sequence
  source: /note="Description of Artificial Sequence: Syntheticoligonucleotide"
aattctcaggctagtcgacggatttaccgtcactcg

SEQ ID NO: 9 - DNA, 36 bp, Artificial Sequence
  source: /note="Description of Artificial Sequence: Syntheticoligonucleotide"
cctacaaacctttagcagtcctctgttggtctctgc

SEQ ID NO: 10 - DNA, 36 bp, Artificial Sequence
  source: /note="Description of Artificial Sequence: Syntheticoligonucleotide"
aaggtctccagattcaatggtacgaccatccgactc

SEQ ID NO: 11 - DNA, 36 bp, Artificial Sequence
  source: /note="Description of Artificial Sequence: Syntheticoligonucleotide"
aactgtctgttctctgtgaggaattctccttccgag

SEQ ID NO: 12 - DNA, 36 bp, Artificial Sequence
  source: /note="Description of Artificial Sequence: Syntheticoligonucleotide"
tcacgtaaccattgtcgtttaatgtctcccagcgtt

SEQ ID NO: 13 - DNA, 36 bp, Artificial Sequence
  source: /note="Description of Artificial Sequence: Syntheticoligonucleotide"
ctatgccagatgatgctatcccgacgatgaacggtt

SEQ ID NO: 14 - DNA, 36 bp, Artificial Sequence
  source: /note="Description of Artificial Sequence: Syntheticoligonucleotide"
tccctacgtcacaactacctattcgagtgtggtctt

SEQ ID NO: 15 - DNA, 36 bp, Artificial Sequence
  source: /note="Description of Artificial Sequence: Syntheticoligonucleotide"
gtgctatctagctaaccaagcgggctctttataggc

SEQ ID NO: 16 - DNA, 36 bp, Artificial Sequence
  source: /note="Description of Artificial Sequence: Syntheticoligonucleotide"
atactaagggttactgccatagtgccgacgcgtaga

SEQ ID NO: 17 - DNA, 36 bp, Artificial Sequence
  source: /note="Description of Artificial Sequence: Syntheticoligonucleotide"
aggtgtttctttctagacaccaagtatccacaggca

SEQ ID NO: 18 - DNA, 36 bp, Artificial Sequence
  source: /note="Description of Artificial Sequence: Syntheticoligonucleotide"
cgagacggagcaacgtttgctttcagttaatcggag

SEQ ID NO: 19 - DNA, 36 bp, Artificial Sequence
  source: /note="Description of Artificial Sequence: Syntheticoligonucleotide"
gacggaatcttacataaagtgtttggagatggtagg

SEQ ID NO: 20 - DNA, 36 bp, Artificial Sequence
  source: /note="Description of Artificial Sequence: Syntheticoligonucleotide"
cagtaccgtgatttaagtcgggtagacgatgcggat

SEQ ID NO: 21 - DNA, 36 bp, Artificial Sequence
  source: /note="Description of Artificial Sequence: Syntheticoligonucleotide"
gtctctacgttataccggatttgggtattctctgag

SEQ ID NO: 22 - DNA, 36 bp, Artificial Sequence
  source: /note="Description of Artificial Sequence: Syntheticoligonucleotide"
ctttctttgagactgcgggaaagcggttcggtaact

SEQ ID NO: 23 - DNA, 36 bp, Artificial Sequence
  source: /note="Description of Artificial Sequence: Syntheticoligonucleotide"
gtacttacacggcttggctcagtgcccgtttcatat

SEQ ID NO: 24 - DNA, 36 bp, Artificial Sequence
  source: /note="Description of Artificial Sequence: Syntheticoligonucleotide"
ctcggtgttctgtaggtaaataacgagtaatcgcac

SEQ ID NO: 25 - DNA, 36 bp, Artificial Sequence
  source: /note="Description of Artificial Sequence: Syntheticoligonucleotide"
gtaacatcccaagcgaacctggcctttagtacccaa

SEQ ID NO: 26 - DNA, 36 bp, Artificial Sequence
  source: /note="Description of Artificial Sequence: Syntheticoligonucleotide"
gggtagtgctgcaacagtcgcgaattataaatacgg

SEQ ID NO: 27 - DNA, 36 bp, Artificial Sequence
  source: /note="Description of Artificial Sequence: Syntheticoligonucleotide"
agtcagtcatttatcacacacatacacagtgagctc

SEQ ID NO: 28 - DNA, 36 bp, Artificial Sequence
  source: /note="Description of Artificial Sequence: Syntheticoligonucleotide"
ttcaaagatgctcatcacccttcgcatctcggacca

SEQ ID NO: 29 - DNA, 36 bp, Artificial Sequence
  source: /note="Description of Artificial Sequence: Syntheticoligonucleotide"
atagtctcgagtgcgcctgtccctcttacagtttct

SEQ ID NO: 30 - DNA, 36 bp, Artificial Sequence
  source: /note="Description of Artificial Sequence: Syntheticoligonucleotide"
tcctacgcgatctacctgtattactttactggcaac

SEQ ID NO: 31 - DNA, 36 bp, Artificial Sequence
  source: /note="Description of Artificial Sequence: Syntheticoligonucleotide"
ttctgcggaagctgatccgtcacacaatccttcttg

SEQ ID NO: 32 - DNA, 36 bp, Artificial Sequence
  source: /note="Description of Artificial Sequence: Syntheticoligonucleotide"
tggttccgccgcttgaatacaaccaatacttatcgg

SEQ ID NO: 33 - DNA, 36 bp, Artificial Sequence
  source: /note="Description of Artificial Sequence: Syntheticoligonucleotide"
gtgagcgtttacgagcagtacgcttcaactcaattc

SEQ ID NO: 34 - DNA, 36 bp, Artificial Sequence
  source: /note="Description of Artificial Sequence: Syntheticoligonucleotide"
cagggtgttattgttggaacgccaaatcgtctgaac

SEQ ID NO: 35 - DNA, 36 bp, Artificial Sequence
  source: /note="Description of Artificial Sequence: Syntheticoligonucleotide"
cgctcaaatgaactttcaacatcgaatccttgctgg

SEQ ID NO: 36 - DNA, 36 bp, Artificial Sequence
  source: /note="Description of Artificial Sequence: Syntheticoligonucleotide"
tggtattgggcagatgcttcttaggaattgtgcagt

SEQ ID NO: 37 - DNA, 36 bp, Artificial Sequence
  source: /note="Description of Artificial Sequence: Syntheticoligonucleotide"
atcttgccatgcacgaaatttacgattaggtcagcg

SEQ ID NO: 38 - DNA, 36 bp, Artificial Sequence
  source: /note="Description of Artificial Sequence: Syntheticoligonucleotide"
tatactacttaaacaggaacccgctctccgcaggac

SEQ ID NO: 39 - DNA, 36 bp, Artificial Sequence
  source: /note="Description of Artificial Sequence: Syntheticoligonucleotide"
gtagatccctacattcagaaatgcgtctgttgacgg

SEQ ID NO: 40 - DNA, 36 bp, Artificial Sequence
  source: /note="Description of Artificial Sequence: Syntheticoligonucleotide"
gtgtgctaagtgcgcgaatataatagcaagtagttg

SEQ ID NO: 41 - DNA, 36 bp, Artificial Sequence
  source: /note="Description of Artificial Sequence: Syntheticoligonucleotide"
cgcaagcagataatgagttagttaggcacacatcat

SEQ ID NO: 42 - DNA, 36 bp, Artificial Sequence
  source: /note="Description of Artificial Sequence: Syntheticoligonucleotide"
agtatcacgctcctaggcttgtaacagattggctag

SEQ ID NO: 43 - DNA, 36 bp, Artificial Sequence
  source: /note="Description of Artificial Sequence: Syntheticoligonucleotide"
ccatacaattcgtcaatctatgtgacactgtccacc

SEQ ID NO: 44 - DNA, 36 bp, Artificial Sequence
  source: /note="Description of Artificial Sequence: Syntheticoligonucleotide"
acatgacgggttgctcaacataagacgttactcagc

SEQ ID NO: 45 - DNA, 36 bp, Artificial Sequence
  source: /note="Description of Artificial Sequence: Syntheticoligonucleotide"
cggcggttaataaacgtaacgacatgagtgtccctg

SEQ ID NO: 46 - DNA, 36 bp, Artificial Sequence
  source: /note="Description of Artificial Sequence: Syntheticoligonucleotide"
tttcaagtatttccgacattcctgcctagttccgcg

SEQ ID NO: 47 - DNA, 36 bp, Artificial Sequence
  source: /note="Description of Artificial Sequence: Syntheticoligonucleotide"
ttcgtaactagactagcgtacgtcgctataggtctt

SEQ ID NO: 48 - DNA, 36 bp, Artificial Sequence
  source: /note="Description of Artificial Sequence: Syntheticoligonucleotide"
agtggaattcggtgccgcgtataaatcaactgactg

SEQ ID NO: 49 - DNA, 36 bp, Artificial Sequence
  source: /note="Description of Artificial Sequence: Syntheticoligonucleotide"
tttgcctcatccttacacaagacaccttctcctaca

SEQ ID NO: 50 - DNA, 36 bp, Artificial Sequence
  source: /note="Description of Artificial Sequence: Syntheticoligonucleotide"
gatatcaacaacccaccctgccttatcttcgtgtat

SEQ ID NO: 51 - DNA, 36 bp, Artificial Sequence
  source: /note="Description of Artificial Sequence: Syntheticoligonucleotide"
ccagatatagttaaattagctgcgcgtgtacatacg

SEQ ID NO: 52 - DNA, 36 bp, Artificial Sequence
  source: /note="Description of Artificial Sequence: Syntheticoligonucleotide"
tgaatattacccgtcccgcgaccttcaatgagtcgt

SEQ ID NO: 53 - DNA, 36 bp, Artificial Sequence
  source: /note="Description of Artificial Sequence: Syntheticoligonucleotide"
tcatcaattgggacatgttattcctccgtaagcttg

SEQ ID NO: 54 - DNA, 36 bp, Artificial Sequence
  source: /note="Description of Artificial Sequence: Syntheticoligonucleotide"
ttatagaacaaagtcaagggccgtgcgtaattcggg

SEQ ID NO: 55 - DNA, 36 bp, Artificial Sequence
  source: /note="Description of Artificial Sequence: Syntheticoligonucleotide"
gcctcagtaggccctaagttaacatcagctatgtac

SEQ ID NO: 56 - DNA, 36 bp, Artificial Sequence
  source: /note="Description of Artificial Sequence: Syntheticoligonucleotide"
agcaggccaatggagtggtaattcatcttggcctca

SEQ ID NO: 57 - DNA, 36 bp, Artificial Sequence
  source: /note="Description of Artificial Sequence: Syntheticoligonucleotide"
taagtgtcggctcatatctaaagcagacggacataa

SEQ ID NO: 58 - DNA, 36 bp, Artificial Sequence
  source: /note="Description of Artificial Sequence: Syntheticoligonucleotide"
cattgatatgatgatcctcgagccggtcgaacttac

SEQ ID NO: 59 - DNA, 36 bp, Artificial Sequence
  source: /note="Description of Artificial Sequence: Syntheticoligonucleotide"
ggaagtactggcgtctttcgacgcttatcaaactac

SEQ ID NO: 60 - DNA, 36 bp, Artificial Sequence
  source: /note="Description of Artificial Sequence: Syntheticoligonucleotide"
aatatacgagaaacccataagatagcgcgcattcgt

SEQ ID NO: 61 - DNA, 36 bp, Artificial Sequence
  source: /note="Description of Artificial Sequence: Syntheticoligonucleotide"
aacagctactacctgggtcttaatgcggaggttgga

SEQ ID NO: 62 - DNA, 36 bp, Artificial Sequence
  source: /note="Description of Artificial Sequence: Syntheticoligonucleotide"
cccactgatctaacatgaggccggtgggatttgaaa

SEQ ID NO: 63 - DNA, 36 bp, Artificial Sequence
  source: /note="Description of Artificial Sequence: Syntheticoligonucleotide"
ccgacctcgtactaaccgctgccctgataaagaatt

SEQ ID NO: 64 - DNA, 36 bp, Artificial Sequence
  source: /note="Description of Artificial Sequence: Syntheticoligonucleotide"
cgttcgtgacaaagatccgggcctctctatcatagt

SEQ ID NO: 65 - DNA, 36 bp, Artificial Sequence
  source: /note="Description of Artificial Sequence: Syntheticoligonucleotide"
gtcgtcgttcagagtacgtatcaaaggaatgatcaa

SEQ ID NO: 66 - DNA, 36 bp, Artificial Sequence
  source: /note="Description of Artificial Sequence: Syntheticoligonucleotide"
aacagcacggcctccagatcctctgtctttgcataa

SEQ ID NO: 67 - DNA, 36 bp, Artificial Sequence
  source: /note="Description of Artificial Sequence: Syntheticoligonucleotide"
ttactgaaacgctgagtctttccgcttagctactgg

SEQ ID NO: 68 - DNA, 36 bp, Artificial Sequence
  source: /note="Description of Artificial Sequence: Syntheticoligonucleotide"
attcttcgaacgagccattcaattggtatacggagt

SEQ ID NO: 69 - DNA, 36 bp, Artificial Sequence
  source: /note="Description of Artificial Sequence: Syntheticoligonucleotide"
ttagagagcgcgtgcactatttgtaggtaggtatgc

SEQ ID NO: 70 - DNA, 36 bp, Artificial Sequence
  source: /note="Description of Artificial Sequence: Syntheticoligonucleotide"
atctggtcttgagattaaatagctttgccggttgtg

SEQ ID NO: 71 - DNA, 36 bp, Artificial Sequence
  source: /note="Description of Artificial Sequence: Syntheticoligonucleotide"
tagcttaatctgttcgcacaaagtactgctcgtccc

SEQ ID NO: 72 - DNA, 36 bp, Artificial Sequence
  source: /note="Description of Artificial Sequence: Syntheticoligonucleotide"
ccgcaatttctcaattacgactaaagctctcgcggc

SEQ ID NO: 73 - DNA, 36 bp, Artificial Sequence
  source: /note="Description of Artificial Sequence: Syntheticoligonucleotide"
gaccgcactatatttcagctctcattgggcattgca

SEQ ID NO: 74 - DNA, 36 bp, Artificial Sequence
  source: /note="Description of Artificial Sequence: Syntheticoligonucleotide"
agcggcttgtttagactctattcctgaggacctgac

SEQ ID NO: 75 - DNA, 36 bp, Artificial Sequence
  source: /note="Description of Artificial Sequence: Syntheticoligonucleotide"
cataccaacgacagtccaatcactttccttcactcc

SEQ ID NO: 76 - DNA, 36 bp, Artificial Sequence
  source: /note="Description of Artificial Sequence: Syntheticoligonucleotide"
tatatcgaaatcaccgcaacgactcgctttctcatt

SEQ ID NO: 77 - DNA, 36 bp, Artificial Sequence
  source: /note="Description of Artificial Sequence: Syntheticoligonucleotide"
atgtttaaagaaatcccggacggctatgtcaagcgg

SEQ ID NO: 78 - DNA, 36 bp, Artificial Sequence
  source: /note="Description of Artificial Sequence: Syntheticoligonucleotide"
cagctatactatcatgtgttgatcggagaccgctga

SEQ ID NO: 79 - DNA, 36 bp, Artificial Sequence
  source: /note="Description of Artificial Sequence: Syntheticoligonucleotide"
aataacagcgtggtatatcatccgacgtgtctatct

SEQ ID NO: 80 - DNA, 36 bp, Artificial Sequence
  source: /note="Description of Artificial Sequence: Syntheticoligonucleotide"
gtatgtagaaggtcaactgcagcgagcgaattcctt

SEQ ID NO: 81 - DNA, 36 bp, Artificial Sequence
  source: /note="Description of Artificial Sequence: Syntheticoligonucleotide"
tcgctatttgacttgtataggtccctccactaggta

SEQ ID NO: 82 - DNA, 36 bp, Artificial Sequence
  source: /note="Description of Artificial Sequence: Syntheticoligonucleotide"
aaagtgggttggtacccgattcttatcaaactctac

SEQ ID NO: 83 - DNA, 36 bp, Artificial Sequence
  source: /note="Description of Artificial Sequence: Syntheticoligonucleotide"
gacgcattagttgtctagaaccatcatcaacctgtc

SEQ ID NO: 84 - DNA, 36 bp, Artificial Sequence
  source: /note="Description of Artificial Sequence: Syntheticoligonucleotide"
actattaacaccatcagggtcaatgccatggaaggt

SEQ ID NO: 85 - DNA, 36 bp, Artificial Sequence
  source: /note="Description of Artificial Sequence: Syntheticoligonucleotide"
tacgagttaggtccatgtgaacgcataggctgcgaa

SEQ ID NO: 86 - DNA, 36 bp, Artificial Sequence
  source: /note="Description of Artificial Sequence: Syntheticoligonucleotide"
tagtcaaggtctttcacctgttgccgctgtatatat

SEQ ID NO: 87 - DNA, 36 bp, Artificial Sequence
  source: /note="Description of Artificial Sequence: Syntheticoligonucleotide"
atattattagcacgcccgagtattgcttaggccgac

SEQ ID NO: 88 - DNA, 36 bp, Artificial Sequence
  source: /note="Description of Artificial Sequence: Syntheticoligonucleotide"
atgaatctacgtggcgttgtgtcgggtatcgtcgat

SEQ ID NO: 89 - DNA, 36 bp, Artificial Sequence
  source: /note="Description of Artificial Sequence: Syntheticoligonucleotide"
agttactcaccgtggacgaataaacattgctagccc

SEQ ID NO: 90 - DNA, 36 bp, Artificial Sequence
  source: /note="Description of Artificial Sequence: Syntheticoligonucleotide"
ccctcgcgaaacttgaagctacagacatgtgcatga

SEQ ID NO: 91 - DNA, 36 bp, Artificial Sequence
  source: /note="Description of Artificial Sequence: Syntheticoligonucleotide"
gactgattcctctacttactgtctggatggacaggt

SEQ ID NO: 92 - DNA, 36 bp, Artificial Sequence
  source: /note="Description of Artificial Sequence: Syntheticoligonucleotide"
acaatcaggcaggatgacaccaatctggctagactc

SEQ ID NO: 93 - DNA, 36 bp, Artificial Sequence
  source: /note="Description of Artificial Sequence: Syntheticoligonucleotide"
tggagcgtaagccttggagccttgatctagaatgaa

SEQ ID NO: 94 - DNA, 36 bp, Artificial Sequence
  source: /note="Description of Artificial Sequence: Syntheticoligonucleotide"
cgaagcgtcttaaccttagaactttccagtgagtgg

SEQ ID NO: 95 - DNA, 36 bp, Artificial Sequence
  source: /note="Description of Artificial Sequence: Syntheticoligonucleotide"
cgaacattcagggttctggttcgtcagtcgcctaaa

SEQ ID NO: 96 - DNA, 36 bp, Artificial Sequence
  source: /note="Description of Artificial Sequence: Syntheticoligonucleotide"
tcctcaatcgctctacatccgaggagcaagatacaa

SEQ ID NO: 97 - DNA, 36 bp, Artificial Sequence
  source: /note="Description of Artificial Sequence: Syntheticoligonucleotide"
tgtgttgggacggtaatgaggacacaatcgatcagt

SEQ ID NO: 98 - DNA, 36 bp, Artificial Sequence
  source: /note="Description of Artificial Sequence: Syntheticoligonucleotide"
agcttactcaaacaagttagcactgaaggctacaca

SEQ ID NO: 99 - DNA, 36 bp, Artificial Sequence
  source: /note="Description of Artificial Sequence: Syntheticoligonucleotide"
tcgaattgcagcaccgaccttgtgagtcctaaacat

SEQ ID NO: 100 - DNA, 36 bp, Artificial Sequence
  source: /note="Description of Artificial Sequence: Syntheticoligonucleotide"
agcgaagtgagagagaatggtgatccgtgtgattat

SEQ ID NO: 101 - DNA, 36 bp, Artificial Sequence
  source: /note="Description of Artificial Sequence: Syntheticoligonucleotide"
tggaccggaagggttaatcgtatgcggcatgaacaa

SEQ ID NO: 102 - DNA, 36 bp, Artificial Sequence
  source: /note="Description of Artificial Sequence: Syntheticoligonucleotide"
aatagggactctaactcaatctcgtgacagcataca

SEQ ID NO: 103 - DNA, 36 bp, Artificial Sequence
  source: /note="Description of Artificial Sequence: Syntheticoligonucleotide"
aggctcgtcaaatggtcaaaccttcacaaacaactc

SEQ ID NO: 104 - DNA, 36 bp, Artificial Sequence
  source: /note="Description of Artificial Sequence: Syntheticoligonucleotide"
ggaccgttctactcgacgaacttacacttggtcgta

SEQ ID NO: 105 - DNA, 36 bp, Artificial Sequence
  source: /note="Description of Artificial Sequence: Syntheticoligonucleotide"
tgcgacagttgctacatgtcctcttaccacccttca

SEQ ID NO: 106 - DNA, 36 bp, Artificial Sequence
  source: /note="Description of Artificial Sequence: Syntheticoligonucleotide"
gccgtgaatcgtgctttggatgctcaatatacacta

SEQ ID NO: 107 - DNA, 36 bp, Artificial Sequence
  source: /note="Description of Artificial Sequence: Syntheticoligonucleotide"
ctatctgctactcagagaaacgaggttcaggatctc

SEQ ID NO: 108 - DNA, 36 bp, Artificial Sequence
  source: /note="Description of Artificial Sequence: Syntheticoligonucleotide"
gatcctgggattattgatgtggcacccaaacgcaag

SEQ ID NO: 109 - DNA, 36 bp, Artificial Sequence
  source: /note="Description of Artificial Sequence: Syntheticoligonucleotide"
gcggaaccacagctttcttaggttgcatcaatttag

SEQ ID NO: 110 - DNA, 36 bp, Artificial Sequence
  source: /note="Description of Artificial Sequence: Syntheticoligonucleotide"
atctgtgcggtagatgcacgtcattagtctactata

SEQ ID NO: 111 - DNA, 36 bp, Artificial Sequence
  source: /note="Description of Artificial Sequence: Syntheticoligonucleotide"
ccgacgttattcgattcgggaaacagactgtgcttc

SEQ ID NO: 112 - DNA, 36 bp, Artificial Sequence
  source: /note="Description of Artificial Sequence: Syntheticoligonucleotide"
tctggcgctgggtagtaacgtaacacagtttaatta

SEQ ID NO: 113 - DNA, 36 bp, Artificial Sequence
  source: /note="Description of Artificial Sequence: Syntheticoligonucleotide"
agtgggcgcagaacaaccgcagttaagataacacta

SEQ ID NO: 114 - DNA, 36 bp, Artificial Sequence
  source: /note="Description of Artificial Sequence: Syntheticoligonucleotide"
cgtacgtagggacaccgacatgagatataacataga

SEQ ID NO: 115 - DNA, 36 bp, Artificial Sequence
  source: /note="Description of Artificial Sequence: Syntheticoligonucleotide"
catttcgccgtcttcgtaacaacaacggcgtttcgt

SEQ ID NO: 116 - DNA, 36 bp, Artificial Sequence
  source: /note="Description of Artificial Sequence: Syntheticoligonucleotide"
acgagtgacggagtgactgggtttggaattatgctt

SEQ ID NO: 117 - DNA, 36 bp, Artificial Sequence
  source: /note="Description of Artificial Sequence: Syntheticoligonucleotide"
gtacttcagcgcggtgcgtgtagcatgagaattatc

SEQ ID NO: 118 - DNA, 36 bp, Artificial Sequence
  source: /note="Description of Artificial Sequence: Syntheticoligonucleotide"
tggctcttgaacacgtaacgaactatcaatgcggtt

SEQ ID NO: 119 - DNA, 36 bp, Artificial Sequence
  source: /note="Description of Artificial Sequence: Syntheticoligonucleotide"
ttaaacaataagatcccagaacggagcctggcccaa

SEQ ID NO: 120 - DNA, 36 bp, Artificial Sequence
  source: /note="Description of Artificial Sequence: Syntheticoligonucleotide"
atgaacactctccatcttgcactaagtcaggaagca

SEQ ID NO: 121 - DNA, 36 bp, Artificial Sequence
  source: /note="Description of Artificial Sequence: Syntheticoligonucleotide"
ccctcagattctgtgattccactttataggacacgg

SEQ ID NO: 122 - DNA, 36 bp, Artificial Sequence
  source: /note="Description of Artificial Sequence: Syntheticoligonucleotide"
gacatcatatacgtgaacagcaggagaaccaatacg

SEQ ID NO: 123 - DNA, 36 bp, Artificial Sequence
  source: /note="Description of Artificial Sequence: Syntheticoligonucleotide"
gctactgtggagagggtttgtgaatctaggagcaca

SEQ ID NO: 124 - DNA, 36 bp, Artificial Sequence
  source: /note="Description of Artificial Sequence: Syntheticoligonucleotide"
acagcaatgctagggagcaataaacataaccatcca

SEQ ID NO: 125 - DNA, 36 bp, Artificial Sequence
  source: /note="Description of Artificial Sequence: Syntheticoligonucleotide"
gagacgttctctttccatttggatcatttcggaccg

SEQ ID NO: 126 - DNA, 36 bp, Artificial Sequence
  source: /note="Description of Artificial Sequence: Syntheticoligonucleotide"
aaccagaagttgtggcccatactcgtttactgggtg

SEQ ID NO: 127 - DNA, 36 bp, Artificial Sequence
  source: /note="Description of Artificial Sequence: Syntheticoligonucleotide"
gcgaatcgtaactccaccacagagtacgacgatttg

SEQ ID NO: 128 - DNA, 36 bp, Artificial Sequence
  source: /note="Description of Artificial Sequence: Syntheticoligonucleotide"
cggtcgtagcccttatattcgctcagtacgattgac

SEQ ID NO: 129 - DNA, 36 bp, Artificial Sequence
  source: /note="Description of Artificial Sequence: Syntheticoligonucleotide"
ggttccgcttgcgaccgtttagatgtttcagaacag

SEQ ID NO: 130 - DNA, 36 bp, Artificial Sequence
  source: /note="Description of Artificial Sequence: Syntheticoligonucleotide"
ggctgttcgcgtgatacgtcgtaaacctagatagtc

SEQ ID NO: 131 - DNA, 36 bp, Artificial Sequence
  source: /note="Description of Artificial Sequence: Syntheticoligonucleotide"
aaccatgtctagtatttgtcacgtcctgtatgaccg

SEQ ID NO: 132 - DNA, 36 bp, Artificial Sequence
  source: /note="Description of Artificial Sequence: Syntheticoligonucleotide"
tgtacttcgccacacctgtccttgtggtttgcctaa

SEQ ID NO: 133 - DNA, 36 bp, Artificial Sequence
  source: /note="Description of Artificial Sequence: Syntheticoligonucleotide"
actagggtccttcagagccggtagatgtatggcata

SEQ ID NO: 134 - DNA, 36 bp, Artificial Sequence
  source: /note="Description of Artificial Sequence: Syntheticoligonucleotide"
tttacactggtatagggacggtgtgtagccgagcta

SEQ ID NO: 135 - DNA, 36 bp, Artificial Sequence
  source: /note="Description of Artificial Sequence: Syntheticoligonucleotide"
gtgggttaaatatgatttggaggacgagacgcgcat

SEQ ID NO: 136 - DNA, 36 bp, Artificial Sequence
  source: /note="Description of Artificial Sequence: Syntheticoligonucleotide"
aaagttctgacggctattcgcagtttccacggaact

SEQ ID NO: 137 - DNA, 36 bp, Artificial Sequence
  source: /note="Description of Artificial Sequence: Syntheticoligonucleotide"
agattgccttcgtgtgcagaatagcggcatcgtctt

SEQ ID NO: 138 - DNA, 36 bp, Artificial Sequence
  source: /note="Description of Artificial Sequence: Syntheticoligonucleotide"
attccaaggcgatacgggctcgtcttaacgggaatt

SEQ ID NO: 139 - DNA, 36 bp, Artificial Sequence
  source: /note="Description of Artificial Sequence: Syntheticoligonucleotide"
tataagcctcgcctgacagacgttattgtctacaca

SEQ ID NO: 140 - DNA, 36 bp, Artificial Sequence
  source: /note="Description of Artificial Sequence: Syntheticoligonucleotide"
tgctggcttaacgcccatctacacttagctatagaa

SEQ ID NO: 141 - DNA, 36 bp, Artificial Sequence
  source: /note="Description of Artificial Sequence: Syntheticoligonucleotide"
cgggaggttataaaccgcacatagtaaatagctcaa

SEQ ID NO: 142 - DNA, 36 bp, Artificial Sequence
  source: /note="Description of Artificial Sequence: Syntheticoligonucleotide"
atcagttcgcctgtgagcagcagctaatacctgtaa

SEQ ID NO: 143 - DNA, 36 bp, Artificial Sequence
  source: /note="Description of Artificial Sequence: Syntheticoligonucleotide"
aaaggtatcaactacgtatacctgggatgaacagac

SEQ ID NO: 144 - DNA, 36 bp, Artificial Sequence
  source: /note="Description of Artificial Sequence: Syntheticoligonucleotide"
ctccagggatagtttctatgagtttgaacaacgtcg

SEQ ID NO: 145 - DNA, 36 bp, Artificial Sequence
  source: /note="Description of Artificial Sequence: Syntheticoligonucleotide"
ctctatttgacgaactgtctgtaagcacccaaggat

SEQ ID NO: 146 - DNA, 36 bp, Artificial Sequence
  source: /note="Description of Artificial Sequence: Syntheticoligonucleotide"
gagttatatgaaggaaagtgtctcggccgtactttc

SEQ ID NO: 147 - DNA, 36 bp, Artificial Sequence
  source: /note="Description of Artificial Sequence: Syntheticoligonucleotide"
gtctggtcgtgtacccacaaatatagggctgtcttg

SEQ ID NO: 148 - DNA, 36 bp, Artificial Sequence
  source: /note="Description of Artificial Sequence: Syntheticoligonucleotide"
aacatccagatagcgaaaccagtctttactttggcc

SEQ ID NO: 149 - DNA, 36 bp, Artificial Sequence
  source: /note="Description of Artificial Sequence: Syntheticoligonucleotide"
actaaagcgctcgatccaccatttcttgaactgcaa

SEQ ID NO: 150 - DNA, 36 bp, Artificial Sequence
  source: /note="Description of Artificial Sequence: Syntheticoligonucleotide"
ggacatgtagtctaacactgggcgtcataggattgc

SEQ ID NO: 151 - DNA, 36 bp, Artificial Sequence
  source: /note="Description of Artificial Sequence: Syntheticoligonucleotide"
atcttcgaactcgcttcaacctggactgtgctgtta

SEQ ID NO: 152 - DNA, 36 bp, Artificial Sequence
  source: /note="Description of Artificial Sequence: Syntheticoligonucleotide"
tcccgtgctcaattgcgattactacaaagagtagcc

SEQ ID NO: 153 - DNA, 36 bp, Artificial Sequence
  source: /note="Description of Artificial Sequence: Syntheticoligonucleotide"
tcaatttctcgccggagtttgccactgcttcctatg

SEQ ID NO: 154 - DNA, 36 bp, Artificial Sequence
  source: /note="Description of Artificial Sequence: Syntheticoligonucleotide"
atcactatactatggacgcatggagagtgggtatcc

SEQ ID NO: 155 - DNA, 36 bp, Artificial Sequence
  source: /note="Description of Artificial Sequence: Syntheticoligonucleotide"
cacggtttgattagatgcaatagcgttggctgaatg

SEQ ID NO: 156 - DNA, 36 bp, Artificial Sequence
  source: /note="Description of Artificial Sequence: Syntheticoligonucleotide"
ctactctctgaatacattatccgagtgggcgaggtt

SEQ ID NO: 157 - DNA, 36 bp, Artificial Sequence
  source: /note="Description of Artificial Sequence: Syntheticoligonucleotide"
ccgctggtaagttgattgtgcaacccgtaaccttta

SEQ ID NO: 158 - DNA, 36 bp, Artificial Sequence
  source: /note="Description of Artificial Sequence: Syntheticoligonucleotide"
aggaataaagcgacataagaagagcatgcactcttg

SEQ ID NO: 159 - DNA, 36 bp, Artificial Sequence
  source: /note="Description of Artificial Sequence: Syntheticoligonucleotide"
ctgactcctaagtgatgagaacatatagcccacagg

SEQ ID NO: 160 - DNA, 36 bp, Artificial Sequence
  source: /note="Description of Artificial Sequence: Syntheticoligonucleotide"
aatcgttcgttagtgctacgccttcacttaagctat

SEQ ID NO: 161 - DNA, 36 bp, Artificial Sequence
  source: /note="Description of Artificial Sequence: Syntheticoligonucleotide"
caacctgtatcggagaccatttgtaatcacatcgcc

SEQ ID NO: 162 - DNA, 36 bp, Artificial Sequence
  source: /note="Description of Artificial Sequence: Syntheticoligonucleotide"
atcaacgtttgcaataagattcagctggagtagagc

SEQ ID NO: 163 - DNA, 36 bp, Artificial Sequence
  source: /note="Description of Artificial Sequence: Syntheticoligonucleotide"
actatgctccggtaatgggtcattagattcgaagga

SEQ ID NO: 164 - DNA, 36 bp, Artificial Sequence
  source: /note="Description of Artificial Sequence: Syntheticoligonucleotide"
ctacatcgacgaatgctttgtccactattaacgtcg

SEQ ID NO: 165 - DNA, 36 bp, Artificial Sequence
  source: /note="Description of Artificial Sequence: Syntheticoligonucleotide"
ttatcgtggtgtgataactgatttgctttcgggagt

SEQ ID NO: 166 - DNA, 36 bp, Artificial Sequence
  source: /note="Description of Artificial Sequence: Syntheticoligonucleotide"
ggatctacagtgactctatcgggttgggtagttctt

SEQ ID NO: 167 - DNA, 36 bp, Artificial Sequence
  source: /note="Description of Artificial Sequence: Syntheticoligonucleotide"
attcctgaccggatggctgtaggacatagttgtaag

SEQ ID NO: 168 - DNA, 36 bp, Artificial Sequence
  source: /note="Description of Artificial Sequence: Syntheticoligonucleotide"
atgctgacgctgaggtacgctaacaggacaaatcca

SEQ ID NO: 169 - DNA, 36 bp, Artificial Sequence
  source: /note="Description of Artificial Sequence: Syntheticoligonucleotide"
acaattagcggccatatctgttaagtcattcctccg

SEQ ID NO: 170 - DNA, 36 bp, Artificial Sequence
  source: /note="Description of Artificial Sequence: Syntheticoligonucleotide"
tgcataaagaatcctcggagtagttggatcctgatg

SEQ ID NO: 171 - DNA, 36 bp, Artificial Sequence
  source: /note="Description of Artificial Sequence: Syntheticoligonucleotide"
ggacaggccagttaaacattgcgggaagcttaacta

SEQ ID NO: 172 - DNA, 36 bp, Artificial Sequence
  source: /note="Description of Artificial Sequence: Syntheticoligonucleotide"
tttgcgcccggtggttaatccctaatagatctcact

SEQ ID NO: 173 - DNA, 36 bp, Artificial Sequence
  source: /note="Description of Artificial Sequence: Syntheticoligonucleotide"
ggttggtgtctgcaaattgctggcgttggtaatctg

SEQ ID NO: 174 - DNA, 36 bp, Artificial Sequence
  source: /note="Description of Artificial Sequence: Syntheticoligonucleotide"
acgctgtatctccggctgtcaatatgtgaattccgc

SEQ ID NO: 175 - DNA, 36 bp, Artificial Sequence
  source: /note="Description of Artificial Sequence: Syntheticoligonucleotide"
tccactttagtctgcagtcggtgctctcttactcta

SEQ ID NO: 176 - DNA, 36 bp, Artificial Sequence
  source: /note="Description of Artificial Sequence: Syntheticoligonucleotide"
agtaattaaggcttcccattgatccgccgagcatta

SEQ ID NO: 177 - DNA, 36 bp, Artificial Sequence
  source: /note="Description of Artificial Sequence: Syntheticoligonucleotide"
cagaatataccttcggtagcacagcagaccttaggt

SEQ ID NO: 178 - DNA, 36 bp, Artificial Sequence
  source: /note="Description of Artificial Sequence: Syntheticoligonucleotide"
ccgaaactgttgatcatcgcgctttcaaacgggtta

SEQ ID NO: 179 - DNA, 36 bp, Artificial Sequence
  source: /note="Description of Artificial Sequence: Syntheticoligonucleotide"
atgactcggcgatcttgtctgggagctagcaaattc

SEQ ID NO: 180 - DNA, 36 bp, Artificial Sequence
  source: /note="Description of Artificial Sequence: Syntheticoligonucleotide"
gggtcactacgttaaagtgttggtatggccctctaa

SEQ ID NO: 181 - DNA, 36 bp, Artificial Sequence
  source: /note="Description of Artificial Sequence: Syntheticoligonucleotide"
ttcaacaccgttatggatccgtgccgaatcagatcg

SEQ ID NO: 182 - DNA, 36 bp, Artificial Sequence
  source: /note="Description of Artificial Sequence: Syntheticoligonucleotide"
tcagtctgtatggagtatcggcacttccacatcctg

SEQ ID NO: 183 - DNA, 36 bp, Artificial Sequence
  source: /note="Description of Artificial Sequence: Syntheticoligonucleotide"
tgcgggcaatagtagcttggatctcgtgcaattagg

SEQ ID NO: 184 - DNA, 36 bp, Artificial Sequence
  source: /note="Description of Artificial Sequence: Syntheticoligonucleotide"
aattccggtttaccgtcgctcacatttcctggagac

SEQ ID NO: 185 - DNA, 36 bp, Artificial Sequence
  source: /note="Description of Artificial Sequence: Syntheticoligonucleotide"
agttgtgttgtgcgaaattaggcggatgctacggga

SEQ ID NO: 186 - DNA, 36 bp, Artificial Sequence
  source: /note="Description of Artificial Sequence: Syntheticoligonucleotide"
acgttgcctggctgagtgtgttaatgatgtctcgat

SEQ ID NO: 187 - DNA, 36 bp, Artificial Sequence
  source: /note="Description of Artificial Sequence: Syntheticoligonucleotide"
gagacacgactaactggtaacccagacatgatcggt

SEQ ID NO: 188 - DNA, 36 bp, Artificial Sequence
  source: /note="Description of Artificial Sequence: Syntheticoligonucleotide"
atggagggcttgcatttggtcattggttcaagacga

SEQ ID NO: 189 - DNA, 36 bp, Artificial Sequence
  source: /note="Description of Artificial Sequence: Syntheticoligonucleotide"
cctttcgctacactaatagctcctgtgccctcgtat

SEQ ID NO: 190 - DNA, 36 bp, Artificial Sequence
  source: /note="Description of Artificial Sequence: Syntheticoligonucleotide"
taaactgatgcggtggcaacagaaatcaggtggtga

SEQ ID NO: 191 - DNA, 36 bp, Artificial Sequence
  source: /note="Description of Artificial Sequence: Syntheticoligonucleotide"
acatgtcatgaatagatgccgctgcaggtatggaaa

SEQ ID NO: 192 - DNA, 36 bp, Artificial Sequence
  source: /note="Description of Artificial Sequence: Syntheticoligonucleotide"
gtttagactccaactccctgtgggtgagcttaatgg

SEQ ID NO: 193 - DNA, 36 bp, Artificial Sequence
  source: /note="Description of Artificial Sequence: Syntheticoligonucleotide"
tatatgcactctctggtccgttgtggtccttctaac

SEQ ID NO: 194 - DNA, 36 bp, Artificial Sequence
  source: /note="Description of Artificial Sequence: Syntheticoligonucleotide"
cgagtgacggtaaatccgtcgactagcctgagaatt

SEQ ID NO: 195 - DNA, 36 bp, Artificial Sequence
  source: /note="Description of Artificial Sequence: Syntheticoligonucleotide"
gcagagaccaacagaggactgctaaaggtttgtagg

SEQ ID NO: 196 - DNA, 36 bp, Artificial Sequence
  source: /note="Description of Artificial Sequence: Syntheticoligonucleotide"
gagtcggatggtcgtaccattgaatctggagacctt

SEQ ID NO: 197 - DNA, 36 bp, Artificial Sequence
  source: /note="Description of Artificial Sequence: Syntheticoligonucleotide"
ctcggaaggagaattcctcacagagaacagacagtt

SEQ ID NO: 198 - DNA, 36 bp, Artificial Sequence
  source: /note="Description of Artificial Sequence: Syntheticoligonucleotide"
aacgctgggagacattaaacgacaatggttacgtga

SEQ ID NO: 199 - DNA, 36 bp, Artificial Sequence
  source: /note="Description of Artificial Sequence: Syntheticoligonucleotide"
aaccgttcatcgtcgggatagcatcatctggcatag

SEQ ID NO: 200 - DNA, 36 bp, Artificial Sequence
  source: /note="Description of Artificial Sequence: Syntheticoligonucleotide"
aagaccacactcgaataggtagttgtgacgtaggga

SEQ ID NO: 201 - DNA, 36 bp, Artificial Sequence
  source: /note="Description of Artificial Sequence: Syntheticoligonucleotide"
gcctataaagagcccgcttggttagctagatagcac

SEQ ID NO: 202 - DNA, 36 bp, Artificial Sequence
  source: /note="Description of Artificial Sequence: Syntheticoligonucleotide"
tctacgcgtcggcactatggcagtaacccttagtat

SEQ ID NO: 203 - DNA, 36 bp, Artificial Sequence
  source: /note="Description of Artificial Sequence: Syntheticoligonucleotide"
tgcctgtggatacttggtgtctagaaagaaacacct

SEQ ID NO: 204 - DNA, 36 bp, Artificial Sequence
  source: /note="Description of Artificial Sequence: Syntheticoligonucleotide"
ctccgattaactgaaagcaaacgttgctccgtctcg

SEQ ID NO: 205 - DNA, 36 bp, Artificial Sequence
  source: /note="Description of Artificial Sequence: Syntheticoligonucleotide"
cctaccatctccaaacactttatgtaagattccgtc

SEQ ID NO: 206 - DNA, 36 bp, Artificial Sequence
  source: /note="Description of Artificial Sequence: Syntheticoligonucleotide"
atccgcatcgtctacccgacttaaatcacggtactg

SEQ ID NO: 207 - DNA, 36 bp, Artificial Sequence
  source: /note="Description of Artificial Sequence: Syntheticoligonucleotide"
ctcagagaatacccaaatccggtataacgtagagac

SEQ ID NO: 208 - DNA, 36 bp, Artificial Sequence
  source: /note="Description of Artificial Sequence: Syntheticoligonucleotide"
agttaccgaaccgctttcccgcagtctcaaagaaag

SEQ ID NO: 209 - DNA, 36 bp, Artificial Sequence
  source: /note="Description of Artificial Sequence: Syntheticoligonucleotide"
atatgaaacgggcactgagccaagccgtgtaagtac

SEQ ID NO: 210 - DNA, 36 bp, Artificial Sequence
  source: /note="Description of Artificial Sequence: Syntheticoligonucleotide"
gtgcgattactcgttatttacctacagaacaccgag

SEQ ID NO: 211 - DNA, 36 bp, Artificial Sequence
  source: /note="Description of Artificial Sequence: Syntheticoligonucleotide"
ttgggtactaaaggccaggttcgcttgggatgttac

SEQ ID NO: 212 - DNA, 36 bp, Artificial Sequence
  source: /note="Description of Artificial Sequence: Syntheticoligonucleotide"
ccgtatttataattcgcgactgttgcagcactaccc

SEQ ID NO: 213 - DNA, 36 bp, Artificial Sequence
  source: /note="Description of Artificial Sequence: Syntheticoligonucleotide"
gagctcactgtgtatgtgtgtgataaatgactgact

SEQ ID NO: 214 - DNA, 36 bp, Artificial Sequence
  source: /note="Description of Artificial Sequence: Syntheticoligonucleotide"
tggtccgagatgcgaagggtgatgagcatctttgaa

SEQ ID NO: 215 - DNA, 36 bp, Artificial Sequence
  source: /note="Description of Artificial Sequence: Syntheticoligonucleotide"
agaaactgtaagagggacaggcgcactcgagactat

SEQ ID NO: 216 - DNA, 36 bp, Artificial Sequence
  source: /note="Description of Artificial Sequence: Syntheticoligonucleotide"
gttgccagtaaagtaatacaggtagatcgcgtagga

SEQ ID NO: 217 - DNA, 36 bp, Artificial Sequence
  source: /note="Description of Artificial Sequence: Syntheticoligonucleotide"
caagaaggattgtgtgacggatcagcttccgcagaa

SEQ ID NO: 218 - DNA, 36 bp, Artificial Sequence
  source: /note="Description of Artificial Sequence: Syntheticoligonucleotide"
ccgataagtattggttgtattcaagcggcggaacca

SEQ ID NO: 219 - DNA, 36 bp, Artificial Sequence
  source: /note="Description of Artificial Sequence: Syntheticoligonucleotide"
gaattgagttgaagcgtactgctcgtaaacgctcac

SEQ ID NO: 220 - DNA, 36 bp, Artificial Sequence
  source: /note="Description of Artificial Sequence: Syntheticoligonucleotide"
gttcagacgatttggcgttccaacaataacaccctg

SEQ ID NO: 221 - DNA, 36 bp, Artificial Sequence
  source: /note="Description of Artificial Sequence: Syntheticoligonucleotide"
ccagcaaggattcgatgttgaaagttcatttgagcg

SEQ ID NO: 222 - DNA, 36 bp, Artificial Sequence
  source: /note="Description of Artificial Sequence: Syntheticoligonucleotide"
actgcacaattcctaagaagcatctgcccaatacca

SEQ ID NO: 223 - DNA, 36 bp, Artificial Sequence
  source: /note="Description of Artificial Sequence: Syntheticoligonucleotide"
cgctgacctaatcgtaaatttcgtgcatggcaagat

SEQ ID NO: 224 - DNA, 36 bp, Artificial Sequence
  source: /note="Description of Artificial Sequence: Syntheticoligonucleotide"
gtcctgcggagagcgggttcctgtttaagtagtata

SEQ ID NO: 225 - DNA, 36 bp, Artificial Sequence
  source: /note="Description of Artificial Sequence: Syntheticoligonucleotide"
ccgtcaacagacgcatttctgaatgtagggatctac

SEQ ID NO: 226 - DNA, 36 bp, Artificial Sequence
  source: /note="Description of Artificial Sequence: Syntheticoligonucleotide"
caactacttgctattatattcgcgcacttagcacac

SEQ ID NO: 227 - DNA, 36 bp, Artificial Sequence
  source: /note="Description of Artificial Sequence: Syntheticoligonucleotide"
atgatgtgtgcctaactaactcattatctgcttgcg

SEQ ID NO: 228 - DNA, 36 bp, Artificial Sequence
  source: /note="Description of Artificial Sequence: Syntheticoligonucleotide"
ctagccaatctgttacaagcctaggagcgtgatact

SEQ ID NO: 229 - DNA, 36 bp, Artificial Sequence
  source: /note="Description of Artificial Sequence: Syntheticoligonucleotide"
ggtggacagtgtcacatagattgacgaattgtatgg

SEQ ID NO: 230 - DNA, 36 bp, Artificial Sequence
  source: /note="Description of Artificial Sequence: Syntheticoligonucleotide"
gctgagtaacgtcttatgttgagcaacccgtcatgt

SEQ ID NO: 231 - DNA, 36 bp, Artificial Sequence
  source: /note="Description of Artificial Sequence: Syntheticoligonucleotide"
cagggacactcatgtcgttacgtttattaaccgccg

SEQ ID NO: 232 - DNA, 36 bp, Artificial Sequence
  source: /note="Description of Artificial Sequence: Syntheticoligonucleotide"
cgcggaactaggcaggaatgtcggaaatacttgaaa

SEQ ID NO: 233 - DNA, 36 bp, Artificial Sequence
  source: /note="Description of Artificial Sequence: Syntheticoligonucleotide"
aagacctatagcgacgtacgctagtctagttacgaa

SEQ ID NO: 234 - DNA, 36 bp, Artificial Sequence
  source: /note="Description of Artificial Sequence: Syntheticoligonucleotide"
cagtcagttgatttatacgcggcaccgaattccact

SEQ ID NO: 235 - DNA, 36 bp, Artificial Sequence
  source: /note="Description of Artificial Sequence: Syntheticoligonucleotide"
tgtaggagaaggtgtcttgtgtaaggatgaggcaaa

SEQ ID NO: 236 - DNA, 36 bp, Artificial Sequence
  source: /note="Description of Artificial Sequence: Syntheticoligonucleotide"
atacacgaagataaggcagggtgggttgttgatatc

SEQ ID NO: 237 - DNA, 36 bp, Artificial Sequence
  source: /note="Description of Artificial Sequence: Syntheticoligonucleotide"
cgtatgtacacgcgcagctaatttaactatatctgg

SEQ ID NO: 238 - DNA, 36 bp, Artificial Sequence
  source: /note="Description of Artificial Sequence: Syntheticoligonucleotide"
acgactcattgaaggtcgcgggacgggtaatattca

SEQ ID NO: 239 - DNA, 36 bp, Artificial Sequence
  source: /note="Description of Artificial Sequence: Syntheticoligonucleotide"
caagcttacggaggaataacatgtcccaattgatga

SEQ ID NO: 240 - DNA, 36 bp, Artificial Sequence
  source: /note="Description of Artificial Sequence: Syntheticoligonucleotide"
cccgaattacgcacggcccttgactttgttctataa

SEQ ID NO: 241 - DNA, 36 bp, Artificial Sequence
  source: /note="Description of Artificial Sequence: Syntheticoligonucleotide"
gtacatagctgatgttaacttagggcctactgaggc

SEQ ID NO: 242 - DNA, 36 bp, Artificial Sequence
  source: /note="Description of Artificial Sequence: Syntheticoligonucleotide"
tgaggccaagatgaattaccactccattggcctgct

SEQ ID NO: 243 - DNA, 36 bp, Artificial Sequence
  source: /note="Description of Artificial Sequence: Syntheticoligonucleotide"
ttatgtccgtctgctttagatatgagccgacactta

SEQ ID NO: 244 - DNA, 36 bp, Artificial Sequence
  source: /note="Description of Artificial Sequence: Syntheticoligonucleotide"
gtaagttcgaccggctcgaggatcatcatatcaatg

SEQ ID NO: 245 - DNA, 36 bp, Artificial Sequence
  source: /note="Description of Artificial Sequence: Syntheticoligonucleotide"
gtagtttgataagcgtcgaaagacgccagtacttcc

SEQ ID NO: 246 - DNA, 36 bp, Artificial Sequence
  source: /note="Description of Artificial Sequence: Syntheticoligonucleotide"
acgaatgcgcgctatcttatgggtttctcgtatatt

SEQ ID NO: 247 - DNA, 36 bp, Artificial Sequence
  source: /note="Description of Artificial Sequence: Syntheticoligonucleotide"
tccaacctccgcattaagacccaggtagtagctgtt

SEQ ID NO: 248 - DNA, 36 bp, Artificial Sequence
  source: /note="Description of Artificial Sequence: Syntheticoligonucleotide"
tttcaaatcccaccggcctcatgttagatcagtggg

SEQ ID NO: 249 - DNA, 36 bp, Artificial Sequence
  source: /note="Description of Artificial Sequence: Syntheticoligonucleotide"
aattctttatcagggcagcggttagtacgaggtcgg

SEQ ID NO: 250 - DNA, 36 bp, Artificial Sequence
  source: /note="Description of Artificial Sequence: Syntheticoligonucleotide"
actatgatagagaggcccggatctttgtcacgaacg

SEQ ID NO: 251 - DNA, 36 bp, Artificial Sequence
  source: /note="Description of Artificial Sequence: Syntheticoligonucleotide"
ttgatcattcctttgatacgtactctgaacgacgac

SEQ ID NO: 252 - DNA, 36 bp, Artificial Sequence
  source: /note="Description of Artificial Sequence: Syntheticoligonucleotide"
ttatgcaaagacagaggatctggaggccgtgctgtt

SEQ ID NO: 253 - DNA, 36 bp, Artificial Sequence
  source: /note="Description of Artificial Sequence: Syntheticoligonucleotide"
ccagtagctaagcggaaagactcagcgtttcagtaa

SEQ ID NO: 254 - DNA, 36 bp, Artificial Sequence
  source: /note="Description of Artificial Sequence: Syntheticoligonucleotide"
actccgtataccaattgaatggctcgttcgaagaat

SEQ ID NO: 255 - DNA, 36 bp, Artificial Sequence
  source: /note="Description of Artificial Sequence: Syntheticoligonucleotide"
gcatacctacctacaaatagtgcacgcgctctctaa

SEQ ID NO: 256 - DNA, 36 bp, Artificial Sequence
  source: /note="Description of Artificial Sequence: Syntheticoligonucleotide"
cacaaccggcaaagctatttaatctcaagaccagat

SEQ ID NO: 257 - DNA, 36 bp, Artificial Sequence
  source: /note="Description of Artificial Sequence: Syntheticoligonucleotide"
gggacgagcagtactttgtgcgaacagattaagcta

SEQ ID NO: 258 - DNA, 36 bp, Artificial Sequence
  source: /note="Description of Artificial Sequence: Syntheticoligonucleotide"
gccgcgagagctttagtcgtaattgagaaattgcgg

SEQ ID NO: 259 - DNA, 36 bp, Artificial Sequence
  source: /note="Description of Artificial Sequence: Syntheticoligonucleotide"
tgcaatgcccaatgagagctgaaatatagtgcggtc

SEQ ID NO: 260 - DNA, 36 bp, Artificial Sequence
  source: /note="Description of Artificial Sequence: Syntheticoligonucleotide"
gtcaggtcctcaggaatagagtctaaacaagccgct

SEQ ID NO: 261 - DNA, 36 bp, Artificial Sequence
  source: /note="Description of Artificial Sequence: Syntheticoligonucleotide"
ggagtgaaggaaagtgattggactgtcgttggtatg

SEQ ID NO: 262 - DNA, 36 bp, Artificial Sequence
  source: /note="Description of Artificial Sequence: Syntheticoligonucleotide"
aatgagaaagcgagtcgttgcggtgatttcgatata

SEQ ID NO: 263 - DNA, 36 bp, Artificial Sequence
  source: /note="Description of Artificial Sequence: Syntheticoligonucleotide"
ccgcttgacatagccgtccgggatttctttaaacat

SEQ ID NO: 264 - DNA, 36 bp, Artificial Sequence
  source: /note="Description of Artificial Sequence: Syntheticoligonucleotide"
tcagcggtctccgatcaacacatgatagtatagctg

SEQ ID NO: 265 - DNA, 36 bp, Artificial Sequence
  source: /note="Description of Artificial Sequence: Syntheticoligonucleotide"
agatagacacgtcggatgatataccacgctgttatt

SEQ ID NO: 266 - DNA, 36 bp, Artificial Sequence
  source: /note="Description of Artificial Sequence: Syntheticoligonucleotide"
aaggaattcgctcgctgcagttgaccttctacatac

SEQ ID NO: 267 - DNA, 36 bp, Artificial Sequence
  source: /note="Description of Artificial Sequence: Syntheticoligonucleotide"
tacctagtggagggacctatacaagtcaaatagcga

SEQ ID NO: 268 - DNA, 36 bp, Artificial Sequence
  source: /note="Description of Artificial Sequence: Syntheticoligonucleotide"
gtagagtttgataagaatcgggtaccaacccacttt

SEQ ID NO: 269 - DNA, 36 bp, Artificial Sequence
  source: /note="Description of Artificial Sequence: Syntheticoligonucleotide"
gacaggttgatgatggttctagacaactaatgcgtc

SEQ ID NO: 270 - DNA, 36 bp, Artificial Sequence
  source: /note="Description of Artificial Sequence: Syntheticoligonucleotide"
accttccatggcattgaccctgatggtgttaatagt

SEQ ID NO: 271 - DNA, 36 bp, Artificial Sequence
  source: /note="Description of Artificial Sequence: Syntheticoligonucleotide"
ttcgcagcctatgcgttcacatggacctaactcgta

SEQ ID NO: 272 - DNA, 36 bp, Artificial Sequence
  source: /note="Description of Artificial Sequence: Syntheticoligonucleotide"
atatatacagcggcaacaggtgaaagaccttgacta

SEQ ID NO: 273 - DNA, 36 bp, Artificial Sequence
  source: /note="Description of Artificial Sequence: Syntheticoligonucleotide"
gtcggcctaagcaatactcgggcgtgctaataatat

SEQ ID NO: 274 - DNA, 36 bp, Artificial Sequence
  source: /note="Description of Artificial Sequence: Syntheticoligonucleotide"
atcgacgatacccgacacaacgccacgtagattcat

SEQ ID NO: 275 - DNA, 36 bp, Artificial Sequence
  source: /note="Description of Artificial Sequence: Syntheticoligonucleotide"
gggctagcaatgtttattcgtccacggtgagtaact

SEQ ID NO: 276 - DNA, 36 bp, Artificial Sequence
  source: /note="Description of Artificial Sequence: Syntheticoligonucleotide"
tcatgcacatgtctgtagcttcaagtttcgcgaggg

SEQ ID NO: 277 - DNA, 36 bp, Artificial Sequence
  source: /note="Description of Artificial Sequence: Syntheticoligonucleotide"
acctgtccatccagacagtaagtagaggaatcagtc

SEQ ID NO: 278 - DNA, 36 bp, Artificial Sequence
  source: /note="Description of Artificial Sequence: Syntheticoligonucleotide"
gagtctagccagattggtgtcatcctgcctgattgt

SEQ ID NO: 279 - DNA, 36 bp, Artificial Sequence
  source: /note="Description of Artificial Sequence: Syntheticoligonucleotide"
ttcattctagatcaaggctccaaggcttacgctcca

SEQ ID NO: 280 - DNA, 36 bp, Artificial Sequence
  source: /note="Description of Artificial Sequence: Syntheticoligonucleotide"
ccactcactggaaagttctaaggttaagacgcttcg

SEQ ID NO: 281 - DNA, 36 bp, Artificial Sequence
  source: /note="Description of Artificial Sequence: Syntheticoligonucleotide"
tttaggcgactgacgaaccagaaccctgaatgttcg

SEQ ID NO: 282 - DNA, 36 bp, Artificial Sequence
  source: /note="Description of Artificial Sequence: Syntheticoligonucleotide"
ttgtatcttgctcctcggatgtagagcgattgagga

SEQ ID NO: 283 - DNA, 36 bp, Artificial Sequence
  source: /note="Description of Artificial Sequence: Syntheticoligonucleotide"
actgatcgattgtgtcctcattaccgtcccaacaca

SEQ ID NO: 284 - DNA, 36 bp, Artificial Sequence
  source: /note="Description of Artificial Sequence: Syntheticoligonucleotide"
tgtgtagccttcagtgctaacttgtttgagtaagct

SEQ ID NO: 285 - DNA, 36 bp, Artificial Sequence
  source: /note="Description of Artificial Sequence: Syntheticoligonucleotide"
atgtttaggactcacaaggtcggtgctgcaattcga

SEQ ID NO: 286 - DNA, 36 bp, Artificial Sequence
  source: /note="Description of Artificial Sequence: Syntheticoligonucleotide"
ataatcacacggatcaccattctctctcacttcgct

SEQ ID NO: 287 - DNA, 36 bp, Artificial Sequence
  source: /note="Description of Artificial Sequence: Syntheticoligonucleotide"
ttgttcatgccgcatacgattaacccttccggtcca

SEQ ID NO: 288 - DNA, 36 bp, Artificial Sequence
  source: /note="Description of Artificial Sequence: Syntheticoligonucleotide"
tgtatgctgtcacgagattgagttagagtccctatt

SEQ ID NO: 289 - DNA, 36 bp, Artificial Sequence
  source: /note="Description of Artificial Sequence: Syntheticoligonucleotide"
gagttgtttgtgaaggtttgaccatttgacgagcct

SEQ ID NO: 290 - DNA, 36 bp, Artificial Sequence
  source: /note="Description of Artificial Sequence: Syntheticoligonucleotide"
tacgaccaagtgtaagttcgtcgagtagaacggtcc

SEQ ID NO: 291 - DNA, 36 bp, Artificial Sequence
  source: /note="Description of Artificial Sequence: Syntheticoligonucleotide"
tgaagggtggtaagaggacatgtagcaactgtcgca

SEQ ID NO: 292 - DNA, 36 bp, Artificial Sequence
  source: /note="Description of Artificial Sequence: Syntheticoligonucleotide"
tagtgtatattgagcatccaaagcacgattcacggc

SEQ ID NO: 293 - DNA, 36 bp, Artificial Sequence
  source: /note="Description of Artificial Sequence: Syntheticoligonucleotide"
gagatcctgaacctcgtttctctgagtagcagatag

SEQ ID NO: 294 - DNA, 36 bp, Artificial Sequence
  source: /note="Description of Artificial Sequence: Syntheticoligonucleotide"
cttgcgtttgggtgccacatcaataatcccaggatc

SEQ ID NO: 295 - DNA, 36 bp, Artificial Sequence
  source: /note="Description of Artificial Sequence: Syntheticoligonucleotide"
ctaaattgatgcaacctaagaaagctgtggttccgc

SEQ ID NO: 296 - DNA, 36 bp, Artificial Sequence
  source: /note="Description of Artificial Sequence: Syntheticoligonucleotide"
tatagtagactaatgacgtgcatctaccgcacagat

SEQ ID NO: 297 - DNA, 36 bp, Artificial Sequence
  source: /note="Description of Artificial Sequence: Syntheticoligonucleotide"
gaagcacagtctgtttcccgaatcgaataacgtcgg

SEQ ID NO: 298 - DNA, 36 bp, Artificial Sequence
  source: /note="Description of Artificial Sequence: Syntheticoligonucleotide"
taattaaactgtgttacgttactacccagcgccaga

SEQ ID NO: 299 - DNA, 36 bp, Artificial Sequence
  source: /note="Description of Artificial Sequence: Syntheticoligonucleotide"
tagtgttatcttaactgcggttgttctgcgcccact

SEQ ID NO: 300 - DNA, 36 bp, Artificial Sequence
  source: /note="Description of Artificial Sequence: Syntheticoligonucleotide"
tctatgttatatctcatgtcggtgtccctacgtacg

SEQ ID NO: 301 - DNA, 36 bp, Artificial Sequence
  source: /note="Description of Artificial Sequence: Syntheticoligonucleotide"
acgaaacgccgttgttgttacgaagacggcgaaatg

SEQ ID NO: 302 - DNA, 36 bp, Artificial Sequence
  source: /note="Description of Artificial Sequence: Syntheticoligonucleotide"
aagcataattccaaacccagtcactccgtcactcgt

SEQ ID NO: 303 - DNA, 36 bp, Artificial Sequence
  source: /note="Description of Artificial Sequence: Syntheticoligonucleotide"
gataattctcatgctacacgcaccgcgctgaagtac

SEQ ID NO: 304 - DNA, 36 bp, Artificial Sequence
  source: /note="Description of Artificial Sequence: Syntheticoligonucleotide"
aaccgcattgatagttcgttacgtgttcaagagcca

SEQ ID NO: 305 - DNA, 36 bp, Artificial Sequence
  source: /note="Description of Artificial Sequence: Syntheticoligonucleotide"
ttgggccaggctccgttctgggatcttattgtttaa

SEQ ID NO: 306 - DNA, 36 bp, Artificial Sequence
  source: /note="Description of Artificial Sequence: Syntheticoligonucleotide"
tgcttcctgacttagtgcaagatggagagtgttcat

SEQ ID NO: 307 - DNA, 36 bp, Artificial Sequence
  source: /note="Description of Artificial Sequence: Syntheticoligonucleotide"
ccgtgtcctataaagtggaatcacagaatctgaggg

SEQ ID NO: 308 - DNA, 36 bp, Artificial Sequence
  source: /note="Description of Artificial Sequence: Syntheticoligonucleotide"
cgtattggttctcctgctgttcacgtatatgatgtc

SEQ ID NO: 309 - DNA, 36 bp, Artificial Sequence
  source: /note="Description of Artificial Sequence: Syntheticoligonucleotide"
tgtgctcctagattcacaaaccctctccacagtagc

SEQ ID NO: 310 - DNA, 36 bp, Artificial Sequence
  source: /note="Description of Artificial Sequence: Syntheticoligonucleotide"
tggatggttatgtttattgctccctagcattgctgt

SEQ ID NO: 311 - DNA, 36 bp, Artificial Sequence
  source: /note="Description of Artificial Sequence: Syntheticoligonucleotide"
cggtccgaaatgatccaaatggaaagagaacgtctc

SEQ ID NO: 312 - DNA, 36 bp, Artificial Sequence
  source: /note="Description of Artificial Sequence: Syntheticoligonucleotide"
cacccagtaaacgagtatgggccacaacttctggtt

SEQ ID NO: 313 - DNA, 36 bp, Artificial Sequence
  source: /note="Description of Artificial Sequence: Syntheticoligonucleotide"
caaatcgtcgtactctgtggtggagttacgattcgc

SEQ ID NO: 314 - DNA, 36 bp, Artificial Sequence
  source: /note="Description of Artificial Sequence: Syntheticoligonucleotide"
gtcaatcgtactgagcgaatataagggctacgaccg

SEQ ID NO: 315 - DNA, 36 bp, Artificial Sequence
  source: /note="Description of Artificial Sequence: Syntheticoligonucleotide"
ctgttctgaaacatctaaacggtcgcaagcggaacc

SEQ ID NO: 316 - DNA, 36 bp, Artificial Sequence
  source: /note="Description of Artificial Sequence: Syntheticoligonucleotide"
gactatctaggtttacgacgtatcacgcgaacagcc

SEQ ID NO: 317 - DNA, 36 bp, Artificial Sequence
  source: /note="Description of Artificial Sequence: Syntheticoligonucleotide"
cggtcatacaggacgtgacaaatactagacatggtt

SEQ ID NO: 318 - DNA, 36 bp, Artificial Sequence
  source: /note="Description of Artificial Sequence: Syntheticoligonucleotide"
ttaggcaaaccacaaggacaggtgtggcgaagtaca

SEQ ID NO: 319 - DNA, 36 bp, Artificial Sequence
  source: /note="Description of Artificial Sequence: Syntheticoligonucleotide"
tatgccatacatctaccggctctgaaggaccctagt

SEQ ID NO: 320 - DNA, 36 bp, Artificial Sequence
  source: /note="Description of Artificial Sequence: Syntheticoligonucleotide"
tagctcggctacacaccgtccctataccagtgtaaa

SEQ ID NO: 321 - DNA, 36 bp, Artificial Sequence
  source: /note="Description of Artificial Sequence: Syntheticoligonucleotide"
atgcgcgtctcgtcctccaaatcatatttaacccac

SEQ ID NO: 322 - DNA, 36 bp, Artificial Sequence
  source: /note="Description of Artificial Sequence: Syntheticoligonucleotide"
agttccgtggaaactgcgaatagccgtcagaacttt

SEQ ID NO: 323 - DNA, 36 bp, Artificial Sequence
  source: /note="Description of Artificial Sequence: Syntheticoligonucleotide"
aagacgatgccgctattctgcacacgaaggcaatct

SEQ ID NO: 324 - DNA, 36 bp, Artificial Sequence
  source: /note="Description of Artificial Sequence: Syntheticoligonucleotide"
aattcccgttaagacgagcccgtatcgccttggaat

SEQ ID NO: 325 - DNA, 36 bp, Artificial Sequence
  source: /note="Description of Artificial Sequence: Syntheticoligonucleotide"
tgtgtagacaataacgtctgtcaggcgaggcttata

SEQ ID NO: 326 - DNA, 36 bp, Artificial Sequence
  source: /note="Description of Artificial Sequence: Syntheticoligonucleotide"
ttctatagctaagtgtagatgggcgttaagccagca

SEQ ID NO: 327 - DNA, 36 bp, Artificial Sequence
  source: /note="Description of Artificial Sequence: Syntheticoligonucleotide"
ttgagctatttactatgtgcggtttataacctcccg

SEQ ID NO: 328 - DNA, 36 bp, Artificial Sequence
  source: /note="Description of Artificial Sequence: Syntheticoligonucleotide"
ttacaggtattagctgctgctcacaggcgaactgat

SEQ ID NO: 329 - DNA, 36 bp, Artificial Sequence
  source: /note="Description of Artificial Sequence: Syntheticoligonucleotide"
gtctgttcatcccaggtatacgtagttgataccttt

SEQ ID NO: 330 - DNA, 36 bp, Artificial Sequence
  source: /note="Description of Artificial Sequence: Syntheticoligonucleotide"
cgacgttgttcaaactcatagaaactatccctggag

SEQ ID NO: 331 - DNA, 36 bp, Artificial Sequence
  source: /note="Description of Artificial Sequence: Syntheticoligonucleotide"
atccttgggtgcttacagacagttcgtcaaatagag

SEQ ID NO: 332 - DNA, 36 bp, Artificial Sequence
  source: /note="Description of Artificial Sequence: Syntheticoligonucleotide"
gaaagtacggccgagacactttccttcatataactc

SEQ ID NO: 333 - DNA, 36 bp, Artificial Sequence
  source: /note="Description of Artificial Sequence: Syntheticoligonucleotide"
caagacagccctatatttgtgggtacacgaccagac

SEQ ID NO: 334 - DNA, 36 bp, Artificial Sequence
  source: /note="Description of Artificial Sequence: Syntheticoligonucleotide"
ggccaaagtaaagactggtttcgctatctggatgtt

SEQ ID NO: 335 - DNA, 36 bp, Artificial Sequence
  source: /note="Description of Artificial Sequence: Syntheticoligonucleotide"
ttgcagttcaagaaatggtggatcgagcgctttagt

SEQ ID NO: 336 - DNA, 36 bp, Artificial Sequence
  source: /note="Description of Artificial Sequence: Syntheticoligonucleotide"
gcaatcctatgacgcccagtgttagactacatgtcc

SEQ ID NO: 337 - DNA, 36 bp, Artificial Sequence
  source: /note="Description of Artificial Sequence: Syntheticoligonucleotide"
taacagcacagtccaggttgaagcgagttcgaagat

SEQ ID NO: 338 - DNA, 36 bp, Artificial Sequence
  source: /note="Description of Artificial Sequence: Syntheticoligonucleotide"
ggctactctttgtagtaatcgcaattgagcacggga

SEQ ID NO: 339 - DNA, 36 bp, Artificial Sequence
  source: /note="Description of Artificial Sequence: Syntheticoligonucleotide"
cataggaagcagtggcaaactccggcgagaaattga

SEQ ID NO: 340 - DNA, 36 bp, Artificial Sequence
  source: /note="Description of Artificial Sequence: Syntheticoligonucleotide"
ggatacccactctccatgcgtccatagtatagtgat

SEQ ID NO: 341 - DNA, 36 bp, Artificial Sequence
  source: /note="Description of Artificial Sequence: Syntheticoligonucleotide"
cattcagccaacgctattgcatctaatcaaaccgtg

SEQ ID NO: 342 - DNA, 36 bp, Artificial Sequence
  source: /note="Description of Artificial Sequence: Syntheticoligonucleotide"
aacctcgcccactcggataatgtattcagagagtag

SEQ ID NO: 343 - DNA, 36 bp, Artificial Sequence
  source: /note="Description of Artificial Sequence: Syntheticoligonucleotide"
taaaggttacgggttgcacaatcaacttaccagcgg

SEQ ID NO: 344 - DNA, 36 bp, Artificial Sequence
  source: /note="Description of Artificial Sequence: Syntheticoligonucleotide"
caagagtgcatgctcttcttatgtcgctttattcct

SEQ ID NO: 345 - DNA, 36 bp, Artificial Sequence
  source: /note="Description of Artificial Sequence: Syntheticoligonucleotide"
cctgtgggctatatgttctcatcacttaggagtcag

SEQ ID NO: 346 - DNA, 36 bp, Artificial Sequence
  source: /note="Description of Artificial Sequence: Syntheticoligonucleotide"
atagcttaagtgaaggcgtagcactaacgaacgatt

SEQ ID NO: 347 - DNA, 36 bp, Artificial Sequence
  source: /note="Description of Artificial Sequence: Syntheticoligonucleotide"
ggcgatgtgattacaaatggtctccgatacaggttg

SEQ ID NO: 348 - DNA, 36 bp, Artificial Sequence
  source: /note="Description of Artificial Sequence: Syntheticoligonucleotide"
gctctactccagctgaatcttattgcaaacgttgat

SEQ ID NO: 349 - DNA, 36 bp, Artificial Sequence
  source: /note="Description of Artificial Sequence: Syntheticoligonucleotide"
tccttcgaatctaatgacccattaccggagcatagt

SEQ ID NO: 350 - DNA, 36 bp, Artificial Sequence
  source: /note="Description of Artificial Sequence: Syntheticoligonucleotide"
cgacgttaatagtggacaaagcattcgtcgatgtag

SEQ ID NO: 351 - DNA, 36 bp, Artificial Sequence
  source: /note="Description of Artificial Sequence: Syntheticoligonucleotide"
actcccgaaagcaaatcagttatcacaccacgataa

SEQ ID NO: 352 - DNA, 36 bp, Artificial Sequence
  source: /note="Description of Artificial Sequence: Syntheticoligonucleotide"
aagaactacccaacccgatagagtcactgtagatcc

SEQ ID NO: 353 - DNA, 36 bp, Artificial Sequence
  source: /note="Description of Artificial Sequence: Syntheticoligonucleotide"
cttacaactatgtcctacagccatccggtcaggaat

SEQ ID NO: 354 - DNA, 36 bp, Artificial Sequence
  source: /note="Description of Artificial Sequence: Syntheticoligonucleotide"
tggatttgtcctgttagcgtacctcagcgtcagcat

SEQ ID NO: 355 - DNA, 36 bp, Artificial Sequence
  source: /note="Description of Artificial Sequence: Syntheticoligonucleotide"
cggaggaatgacttaacagatatggccgctaattgt

SEQ ID NO: 356 - DNA, 36 bp, Artificial Sequence
  source: /note="Description of Artificial Sequence: Syntheticoligonucleotide"
catcaggatccaactactccgaggattctttatgca

SEQ ID NO: 357 - DNA, 36 bp, Artificial Sequence
  source: /note="Description of Artificial Sequence: Syntheticoligonucleotide"
tagttaagcttcccgcaatgtttaactggcctgtcc

SEQ ID NO: 358 - DNA, 36 bp, Artificial Sequence
  source: /note="Description of Artificial Sequence: Syntheticoligonucleotide"
agtgagatctattagggattaaccaccgggcgcaaa

SEQ ID NO: 359 - DNA, 36 bp, Artificial Sequence
  source: /note="Description of Artificial Sequence: Syntheticoligonucleotide"
cagattaccaacgccagcaatttgcagacaccaacc

SEQ ID NO: 360 - DNA, 36 bp, Artificial Sequence
  source: /note="Description of Artificial Sequence: Syntheticoligonucleotide"
gcggaattcacatattgacagccggagatacagcgt

SEQ ID NO: 361 - DNA, 36 bp, Artificial Sequence
  source: /note="Description of Artificial Sequence: Syntheticoligonucleotide"
tagagtaagagagcaccgactgcagactaaagtgga

SEQ ID NO: 362 - DNA, 36 bp, Artificial Sequence
  source: /note="Description of Artificial Sequence: Syntheticoligonucleotide"
taatgctcggcggatcaatgggaagccttaattact

SEQ ID NO: 363 - DNA, 36 bp, Artificial Sequence
  source: /note="Description of Artificial Sequence: Syntheticoligonucleotide"
acctaaggtctgctgtgctaccgaaggtatattctg

SEQ ID NO: 364 - DNA, 36 bp, Artificial Sequence
  source: /note="Description of Artificial Sequence: Syntheticoligonucleotide"
taacccgtttgaaagcgcgatgatcaacagtttcgg

SEQ ID NO: 365 - DNA, 36 bp, Artificial Sequence
  source: /note="Description of Artificial Sequence: Syntheticoligonucleotide"
gaatttgctagctcccagacaagatcgccgagtcat

SEQ ID NO: 366 - DNA, 36 bp, Artificial Sequence
  source: /note="Description of Artificial Sequence: Syntheticoligonucleotide"
ttagagggccataccaacactttaacgtagtgaccc

SEQ ID NO: 367 - DNA, 36 bp, Artificial Sequence
  source: /note="Description of Artificial Sequence: Syntheticoligonucleotide"
cgatctgattcggcacggatccataacggtgttgaa

SEQ ID NO: 368 - DNA, 36 bp, Artificial Sequence
  source: /note="Description of Artificial Sequence: Syntheticoligonucleotide"
caggatgtggaagtgccgatactccatacagactga

SEQ ID NO: 369 - DNA, 36 bp, Artificial Sequence
  source: /note="Description of Artificial Sequence: Syntheticoligonucleotide"
cctaattgcacgagatccaagctactattgcccgca

SEQ ID NO: 370 - DNA, 36 bp, Artificial Sequence
  source: /note="Description of Artificial Sequence: Syntheticoligonucleotide"
gtctccaggaaatgtgagcgacggtaaaccggaatt

SEQ ID NO: 371 - DNA, 36 bp, Artificial Sequence
  source: /note="Description of Artificial Sequence: Syntheticoligonucleotide"
tcccgtagcatccgcctaatttcgcacaacacaact

SEQ ID NO: 372 - DNA, 36 bp, Artificial Sequence
  source: /note="Description of Artificial Sequence: Syntheticoligonucleotide"
atcgagacatcattaacacactcagccaggcaacgt

SEQ ID NO: 373 - DNA, 36 bp, Artificial Sequence
  source: /note="Description of Artificial Sequence: Syntheticoligonucleotide"
ctctgtgctgattgaccattgggtctgtactagcca

SEQ ID NO: 374 - DNA, 36 bp, Artificial Sequence
  source: /note="Description of Artificial Sequence: Syntheticoligonucleotide"
tacctcccgaacgtaaaccagtaaccaagttctgct

SEQ ID NO: 375 - DNA, 36 bp, Artificial Sequence
  source: /note="Description of Artificial Sequence: Syntheticoligonucleotide"
ggaaagcgatgtgattatcgaggacacgggagcata

SEQ ID NO: 376 - DNA, 36 bp, Artificial Sequence
  source: /note="Description of Artificial Sequence: Syntheticoligonucleotide"
atttgactacgccaccgttgtctttagtccaccact

SEQ ID NO: 377 - DNA, 36 bp, Artificial Sequence
  source: /note="Description of Artificial Sequence: Syntheticoligonucleotide"
tgtacagtacttatctacggcgacgtccataccttt

SEQ ID NO: 378 - DNA, 36 bp, Artificial Sequence
  source: /note="Description of Artificial Sequence: Syntheticoligonucleotide"
caaatctgaggttgagggacacccactcgaattacc

SEQ ID NO: 379 - DNA, 36 bp, Artificial Sequence
  source: /note="Description of Artificial Sequence: Syntheticoligonucleotide"
atatacgtgagagaccaggcaacaccaggaagattg

SEQ ID NO: 380 - DNA, 36 bp, Artificial Sequence
  source: /note="Description of Artificial Sequence: Syntheticoligonucleotide"
gctcactgccatttaggcagctgatcggactcttaa

SEQ ID NO: 381 - DNA, 36 bp, Artificial Sequence
  source: /note="Description of Artificial Sequence: Syntheticoligonucleotide"
cgtctctggttgtctcctgacgatttccaaacatcc

SEQ ID NO: 382 - DNA, 36 bp, Artificial Sequence
  source: /note="Description of Artificial Sequence: Syntheticoligonucleotide"
ctcagcctaccagcatggtaacttagacctctggaa

SEQ ID NO: 383 - DNA, 36 bp, Artificial Sequence
  source: /note="Description of Artificial Sequence: Syntheticoligonucleotide"
gagccttcctcttaaggagtgtctcttgtctgtcaa

SEQ ID NO: 384 - DNA, 36 bp, Artificial Sequence
  source: /note="Description of Artificial Sequence: Syntheticoligonucleotide"
ttgcgaccctctgtaatttgctgttaccaatgcact

SEQ ID NO: 385 - DNA, 36 bp, Artificial Sequence
  source: /note="Description of Artificial Sequence: Syntheticoligonucleotide"
ttggcaagtagcagccctatcgtagtagaccgtatc

SEQ ID NO: 386 - DNA, 36 bp, Artificial Sequence
  source: /note="Description of Artificial Sequence: Syntheticoligonucleotide"
ttctggtgtgagcttatccatcaacactgcatccct

SEQ ID NO: 387 - DNA, 36 bp, Artificial Sequence
  source: /note="Description of Artificial Sequence: Syntheticoligonucleotide"
cggatatttctcgggcgaaccaatcgatctatcgtg

SEQ ID NO: 388 - DNA, 36 bp, Artificial Sequence
  source: /note="Description of Artificial Sequence: Syntheticoligonucleotide"
agatgcgcagccgtgataccgtcattgggaatcata

SEQ ID NO: 389 - DNA, 36 bp, Artificial Sequence
  source: /note="Description of Artificial Sequence: Syntheticoligonucleotide"
acggacacctatgaaccacagatctttctttgtgga

SEQ ID NO: 390 - DNA, 36 bp, Artificial Sequence
  source: /note="Description of Artificial Sequence: Syntheticoligonucleotide"
gaggctaattgactttcgtttgcaacgaggcagagc

SEQ ID NO: 391 - DNA, 36 bp, Artificial Sequence
  source: /note="Description of Artificial Sequence: Syntheticoligonucleotide"
ggatggtagaggtttgtgaaatacattctaaggcag

SEQ ID NO: 392 - DNA, 36 bp, Artificial Sequence
  source: /note="Description of Artificial Sequence: Syntheticoligonucleotide"
taggcgtagcagatgggctgaatttagtgccatgac

SEQ ID NO: 393 - DNA, 36 bp, Artificial Sequence
  source: /note="Description of Artificial Sequence: Syntheticoligonucleotide"
gagtctcttatgggtttaggccatattgcatctctg

SEQ ID NO: 394 - DNA, 36 bp, Artificial Sequence
  source: /note="Description of Artificial Sequence: Syntheticoligonucleotide"
tcaatggcttggcgaaagggcgtcagagtttctttc

SEQ ID NO: 395 - DNA, 36 bp, Artificial Sequence
  source: /note="Description of Artificial Sequence: Syntheticoligonucleotide"
tatactttgcccgtgactcggttcggcacattcatg

SEQ ID NO: 396 - DNA, 36 bp, Artificial Sequence
  source: /note="Description of Artificial Sequence: Syntheticoligonucleotide"
cacgctaatgagcaataaatggatgtcttgtggctc

SEQ ID NO: 397 - DNA, 36 bp, Artificial Sequence
  source: /note="Description of Artificial Sequence: Syntheticoligonucleotide"
aacccatgatttccggtccaagcgaaccctacaatg

SEQ ID NO: 398 - DNA, 36 bp, Artificial Sequence
  source: /note="Description of Artificial Sequence: Syntheticoligonucleotide"
ggcataaatattaagcgctgacaacgtcgtgatggg

SEQ ID NO: 399 - DNA, 36 bp, Artificial Sequence
  source: /note="Description of Artificial Sequence: Syntheticoligonucleotide"
ctcgagtgacacatacacacactatttactgactga

SEQ ID NO: 400 - DNA, 36 bp, Artificial Sequence
  source: /note="Description of Artificial Sequence: Syntheticoligonucleotide"
accaggctctacgcttcccactactcgtagaaactt

SEQ ID NO: 401 - DNA, 36 bp, Artificial Sequence
  source: /note="Description of Artificial Sequence: Syntheticoligonucleotide"
tctttgacattctccctgtccgcgtgagctctgata

SEQ ID NO: 402 - DNA, 36 bp, Artificial Sequence
  source: /note="Description of Artificial Sequence: Syntheticoligonucleotide"
caacggtcatttcattatgtccatctagcgcatcct

SEQ ID NO: 403 - DNA, 36 bp, Artificial Sequence
  source: /note="Description of Artificial Sequence: Syntheticoligonucleotide"
gttcttcctaacacactgcctagtcgaaggcgtctt

SEQ ID NO: 404 - DNA, 36 bp, Artificial Sequence
  source: /note="Description of Artificial Sequence: Syntheticoligonucleotide"
ggctattcataaccaacataagttcgccgccttggt

SEQ ID NO: 405 - DNA, 36 bp, Artificial Sequence
  source: /note="Description of Artificial Sequence: Syntheticoligonucleotide"
cttaactcaacttcgcatgacgagcatttgcgagtg

SEQ ID NO: 406 - DNA, 36 bp, Artificial Sequence
  source: /note="Description of Artificial Sequence: Syntheticoligonucleotide"
caagtctgctaaaccgcaaggttgttattgtgggac

SEQ ID NO: 407 - DNA, 36 bp, Artificial Sequence
  source: /note="Description of Artificial Sequence: Syntheticoligonucleotide"
ggtcgttcctaagctacaactttcaagtaaactcgc

SEQ ID NO: 408 - DNA, 36 bp, Artificial Sequence
  source: /note="Description of Artificial Sequence: Syntheticoligonucleotide"
tgacgtgttaaggattcttcgtagacgggttatggt

SEQ ID NO: 409 - DNA, 36 bp, Artificial Sequence
  source: /note="Description of Artificial Sequence: Syntheticoligonucleotide"
gcgactggattagcatttaaagcacgtaccgttcta

SEQ ID NO: 410 - DNA, 36 bp, Artificial Sequence
  source: /note="Description of Artificial Sequence: Syntheticoligonucleotide"
caggacgcctctcgcccaaggacaaattcatcatat

SEQ ID NO: 411 - DNA, 36 bp, Artificial Sequence
  source: /note="Description of Artificial Sequence: Syntheticoligonucleotide"
ggcagttgtctgcgtaaagacttacatccctagatg

SEQ ID NO: 412 - DNA, 36 bp, Artificial Sequence
  source: /note="Description of Artificial Sequence: Syntheticoligonucleotide"
gttgatgaacgataatataagcgcgtgaatcgtgtg

SEQ ID NO: 413 - DNA, 36 bp, Artificial Sequence
  source: /note="Description of Artificial Sequence: Syntheticoligonucleotide"
tactacacacggattgattgagtaatagacgaacgc

SEQ ID NO: 414 - DNA, 36 bp, Artificial Sequence
  source: /note="Description of Artificial Sequence: Syntheticoligonucleotide"
gatcggttagacaatgttcggatcctcgcactatga

SEQ ID NO: 415 - DNA, 36 bp, Artificial Sequence
  source: /note="Description of Artificial Sequence: Syntheticoligonucleotide"
ccacctgtcacagtgtatctaactgcttaacatacc

SEQ ID NO: 416 - DNA, 36 bp, Artificial Sequence
  source: /note="Description of Artificial Sequence: Syntheticoligonucleotide"
cgactcattgcagaatacaactcgttgggcagtaca

SEQ ID NO: 417 - DNA, 36 bp, Artificial Sequence
  source: /note="Description of Artificial Sequence: Syntheticoligonucleotide"
gtccctgtgagtacagcaatgcaaataattggcggc

SEQ ID NO: 418 - DNA, 36 bp, Artificial Sequence
  source: /note="Description of Artificial Sequence: Syntheticoligonucleotide"
gcgccttgatccgtccttacagcctttatgaacttt

SEQ ID NO: 419 - DNA, 36 bp, Artificial Sequence
  source: /note="Description of Artificial Sequence: Syntheticoligonucleotide"
ttctggatatcgctgcatgcgatcagatcaatgctt

SEQ ID NO: 420 - DNA, 36 bp, Artificial Sequence
  source: /note="Description of Artificial Sequence: Syntheticoligonucleotide"
gtcagtcaactaaatatgcgccgtggcttaaggtga

SEQ ID NO: 421 - DNA, 36 bp, Artificial Sequence
  source: /note="Description of Artificial Sequence: Syntheticoligonucleotide"
acatcctcttccacagaacacattcctactccgttt

SEQ ID NO: 422 - DNA, 36 bp, Artificial Sequence
  source: /note="Description of Artificial Sequence: Syntheticoligonucleotide"
tatgtgcttctattccgtcccacccaacaactatag

SEQ ID NO: 423 - DNA, 36 bp, Artificial Sequence
  source: /note="Description of Artificial Sequence: Syntheticoligonucleotide"
gcatacatgtgcgcgtcgattaaattgatatagacc

SEQ ID NO: 424 - DNA, 36 bp, Artificial Sequence
  source: /note="Description of Artificial Sequence: Syntheticoligonucleotide"
tgctgagtaacttccagcgccctgcccattataagt

SEQ ID NO: 425 - DNA, 36 bp, Artificial Sequence
  source: /note="Description of Artificial Sequence: Syntheticoligonucleotide"
gttcgaatgcctccttattgtacagggttaactact

SEQ ID NO: 426 - DNA, 36 bp, Artificial Sequence
  source: /note="Description of Artificial Sequence: Syntheticoligonucleotide"
gggcttaatgcgtgccgggaactgaaacaagatatt

SEQ ID NO: 427 - DNA, 36 bp, Artificial Sequence
  source: /note="Description of Artificial Sequence: Syntheticoligonucleotide"
catgtatcgactacaattgaatcccggatgactccg

SEQ ID NO: 428 - DNA, 36 bp, Artificial Sequence
  source: /note="Description of Artificial Sequence: Syntheticoligonucleotide"
actccggttctacttaatggtgaggtaaccggacga

SEQ ID NO: 429 - DNA, 36 bp, Artificial Sequence
  source: /note="Description of Artificial Sequence: Syntheticoligonucleotide"
aatacaggcagacgaaatctatactcggctgtgaat

SEQ ID NO: 430 - DNA, 36 bp, Artificial Sequence
  source: /note="Description of Artificial Sequence: Syntheticoligonucleotide"
cattcaagctggccgagctcctagtagtatagttac

SEQ ID NO: 431 - DNA, 36 bp, Artificial Sequence
  source: /note="Description of Artificial Sequence: Syntheticoligonucleotide"
catcaaactattcgcagctttctgcggtcatgaagg

SEQ ID NO: 432 - DNA, 36 bp, Artificial Sequence
  source: /note="Description of Artificial Sequence: Syntheticoligonucleotide"
tgcttacgcgcgatagaatacccaaagagcatataa

SEQ ID NO: 433 - DNA, 36 bp, Artificial Sequence
  source: /note="Description of Artificial Sequence: Syntheticoligonucleotide"
aggttggaggcgtaattctgggtccatcatcgacaa

SEQ ID NO: 434 - DNA, 36 bp, Artificial Sequence
  source: /note="Description of Artificial Sequence: Syntheticoligonucleotide"
aaagtttagggtggccggagtacaatctagtcaccc

SEQ ID NO: 435 - DNA, 36 bp, Artificial Sequence
  source: /note="Description of Artificial Sequence: Syntheticoligonucleotide"
ttaagaaatagtcccgtcgccaatcatgctccagcc

SEQ ID NO: 436 - DNA, 36 bp, Artificial Sequence
  source: /note="Description of Artificial Sequence: Syntheticoligonucleotide"
tgatactatctctccgggcctagaaacagtgcttgc

SEQ ID NO: 437 - DNA, 36 bp, Artificial Sequence
  source: /note="Description of Artificial Sequence: Syntheticoligonucleotide"
aactagtaaggaaactatgcatgagacttgctgctg

SEQ ID NO: 438 - DNA, 36 bp, Artificial Sequence
  source: /note="Description of Artificial Sequence: Syntheticoligonucleotide"
aatacgtttctgtctcctagacctccggcacgacaa

SEQ ID NO: 439 - DNA, 36 bp, Artificial Sequence
  source: /note="Description of Artificial Sequence: Syntheticoligonucleotide"
ggtcatcgattcgcctttctgagtcgcaaagtcatt

SEQ ID NO: 440 - DNA, 36 bp, Artificial Sequence
  source: /note="Description of Artificial Sequence: Syntheticoligonucleotide"
tgaggcatatggttaacttaccgagcaagcttctta

SEQ ID NO: 441 - DNA, 36 bp, Artificial Sequence
  source: /note="Description of Artificial Sequence: Syntheticoligonucleotide"
cgtatggatggatgtttatcacgtgcgcgagagatt

SEQ ID NO: 442 - DNA, 36 bp, Artificial Sequence
  source: /note="Description of Artificial Sequence: Syntheticoligonucleotide"
gtgttggccgtttcgataaattagagttctggtcta

SEQ ID NO: 443 - DNA, 36 bp, Artificial Sequence
  source: /note="Description of Artificial Sequence: Syntheticoligonucleotide"
ccctgctcgtcatgaaacacgcttgtctaattcgat

SEQ ID NO: 444 - DNA, 36 bp, Artificial Sequence
  source: /note="Description of Artificial Sequence: Syntheticoligonucleotide"
cggcgctctcgaaatcagcattaactctttaacgcc

SEQ ID NO: 445 - DNA, 36 bp, Artificial Sequence
  source: /note="Description of Artificial Sequence: Syntheticoligonucleotide"
acgttacgggttactctcgactttatatcacgccag

SEQ ID NO: 446 - DNA, 36 bp, Artificial Sequence
  source: /note="Description of Artificial Sequence: Syntheticoligonucleotide"
cagtccaggagtccttatctcagatttgttcggcga

SEQ ID NO: 447 - DNA, 36 bp, Artificial Sequence
  source: /note="Description of Artificial Sequence: Syntheticoligonucleotide"
cctcacttcctttcactaacctgacagcaaccatac

SEQ ID NO: 448 - DNA, 36 bp, Artificial Sequence
  source: /note="Description of Artificial Sequence: Syntheticoligonucleotide"
ttactctttcgctcagcaacgccactaaagctatat

SEQ ID NO: 449 - DNA, 36 bp, Artificial Sequence
  source: /note="Description of Artificial Sequence: Syntheticoligonucleotide"
ggcgaactgtatcggcaggccctaaagaaatttgta

SEQ ID NO: 450 - DNA, 36 bp, Artificial Sequence
  source: /note="Description of Artificial Sequence: Syntheticoligonucleotide"
agtcgccagaggctagttgtgtactatcatatcgac

SEQ ID NO: 451 - DNA, 36 bp, Artificial Sequence
  source: /note="Description of Artificial Sequence: Syntheticoligonucleotide"
tctatctgtgcagcctactatatggtgcgacaataa

SEQ ID NO: 452 - DNA, 36 bp, Artificial Sequence
  source: /note="Description of Artificial Sequence: Syntheticoligonucleotide"
ttccttaagcgagcgacgtcaactggaagatgtatg

SEQ ID NO: 453 - DNA, 36 bp, Artificial Sequence
  source: /note="Description of Artificial Sequence: Syntheticoligonucleotide"
atggatcacctccctggatatgttcagtttatcgct

SEQ ID NO: 454 - DNA, 36 bp, Artificial Sequence
  source: /note="Description of Artificial Sequence: Syntheticoligonucleotide"
catctcaaactattcttagcccatggttgggtgaaa

SEQ ID NO: 455 - DNA, 36 bp, Artificial Sequence
  source: /note="Description of Artificial Sequence: Syntheticoligonucleotide"
ctgtccaactactaccaagatctgttgattacgcag

SEQ ID NO: 456 - DNA, 36 bp, Artificial Sequence
  source: /note="Description of Artificial Sequence: Syntheticoligonucleotide"
tggaaggtaccgtaactgggactaccacaattatca

SEQ ID NO: 457 - DNA, 36 bp, Artificial Sequence
  source: /note="Description of Artificial Sequence: Syntheticoligonucleotide"
aagcgtcggatacgcaagtgtacctggattgagcat

SEQ ID NO: 458 - DNA, 36 bp, Artificial Sequence
  source: /note="Description of Artificial Sequence: Syntheticoligonucleotide"
tatatatgtcgccgttgtccactttctggaactgat

SEQ ID NO: 459 - DNA, 36 bp, Artificial Sequence
  source: /note="Description of Artificial Sequence: Syntheticoligonucleotide"
cagccggattcgttatgagcccgcacgattattata

SEQ ID NO: 460 - DNA, 36 bp, Artificial Sequence
  source: /note="Description of Artificial Sequence: Syntheticoligonucleotide"
tagctgctatgggctgtgttgcggtgcatctaagta

SEQ ID NO: 461 - DNA, 36 bp, Artificial Sequence
  source: /note="Description of Artificial Sequence: Syntheticoligonucleotide"
cccgatcgttacaaataagcaggtgccactcattga

SEQ ID NO: 462 - DNA, 36 bp, Artificial Sequence
  source: /note="Description of Artificial Sequence: Syntheticoligonucleotide"
agtacgtgtacagacatcgaagttcaaagcgctccc

SEQ ID NO: 463 - DNA, 36 bp, Artificial Sequence
  source: /note="Description of Artificial Sequence: Syntheticoligonucleotide"
tggacaggtaggtctgtcattcatctccttagtcag

SEQ ID NO: 464 - DNA, 36 bp, Artificial Sequence
  source: /note="Description of Artificial Sequence: Syntheticoligonucleotide"
ctcagatcggtctaaccacagtaggacggactaaca

SEQ ID NO: 465 - DNA, 36 bp, Artificial Sequence
  source: /note="Description of Artificial Sequence: Syntheticoligonucleotide"
aagtaagatctagttccgaggttccgaatgcgaggt

SEQ ID NO: 466 - DNA, 36 bp, Artificial Sequence
  source: /note="Description of Artificial Sequence: Syntheticoligonucleotide"
ggtgagtgacctttcaagattccaattctgcgaagc

SEQ ID NO: 467 - DNA, 36 bp, Artificial Sequence
  source: /note="Description of Artificial Sequence: Syntheticoligonucleotide"
aaatccgctgactgcttggtcttgggacttacaagc

SEQ ID NO: 468 - DNA, 36 bp, Artificial Sequence
  source: /note="Description of Artificial Sequence: Syntheticoligonucleotide"
aacatagaacgaggagcctacatctcgctaactcct

SEQ ID NO: 469 - DNA, 36 bp, Artificial Sequence
  source: /note="Description of Artificial Sequence: Syntheticoligonucleotide"
tgactagctaacacaggagtaatggcagggttgtgt

SEQ ID NO: 470 - DNA, 36 bp, Artificial Sequence
  source: /note="Description of Artificial Sequence: Syntheticoligonucleotide"
acacatcggaagtcacgattgaacaaactcattcga

SEQ ID NO: 471 - DNA, 36 bp, Artificial Sequence
  source: /note="Description of Artificial Sequence: Syntheticoligonucleotide"
tacaaatcctgagtgttccagccacgacgttaagct

SEQ ID NO: 472 - DNA, 36 bp, Artificial Sequence
  source: /note="Description of Artificial Sequence: Syntheticoligonucleotide"
tattagtgtgcctagtggtaagagagagtgaagcga

SEQ ID NO: 473 - DNA, 36 bp, Artificial Sequence
  source: /note="Description of Artificial Sequence: Syntheticoligonucleotide"
aacaagtacggcgtatgctaattgggaaggccaggt

SEQ ID NO: 474 - DNA, 36 bp, Artificial Sequence
  source: /note="Description of Artificial Sequence: Syntheticoligonucleotide"
acatacgacagtgctctaactcaatctcagggataa

SEQ ID NO: 475 - DNA, 36 bp, Artificial Sequence
  source: /note="Description of Artificial Sequence: Syntheticoligonucleotide"
ctcaacaaacacttccaaactggtaaactgctcgga

SEQ ID NO: 476 - DNA, 36 bp, Artificial Sequence
  source: /note="Description of Artificial Sequence: Syntheticoligonucleotide"
atgctggttcacattcaagcagctcatcttgccagg

SEQ ID NO: 477 - DNA, 36 bp, Artificial Sequence
  source: /note="Description of Artificial Sequence: Syntheticoligonucleotide"
acttcccaccattctcctgtacatcgttgacagcgt

SEQ ID NO: 478 - DNA, 36 bp, Artificial Sequence
  source: /note="Description of Artificial Sequence: Syntheticoligonucleotide"
atcacatataactcgtaggtttcgtgctaagtgccg

SEQ ID NO: 479 - DNA, 36 bp, Artificial Sequence
  source: /note="Description of Artificial Sequence: Syntheticoligonucleotide"
ctctaggacttggagcaaagagactcatcgtctatc

SEQ ID NO: 480 - DNA, 36 bp, Artificial Sequence
  source: /note="Description of Artificial Sequence: Syntheticoligonucleotide"
gaacgcaaacccacggtgtagttattagggtcctag

SEQ ID NO: 481 - DNA, 36 bp, Artificial Sequence
  source: /note="Description of Artificial Sequence: Syntheticoligonucleotide"
gatttaactacgttggattctttcgacaccaaggcg

SEQ ID NO: 482 - DNA, 36 bp, Artificial Sequence
  source: /note="Description of Artificial Sequence: Syntheticoligonucleotide"
atatcatctgattactgcacgtagatggcgtgtcta

SEQ ID NO: 483 - DNA, 36 bp, Artificial Sequence
  source: /note="Description of Artificial Sequence: Syntheticoligonucleotide"
cttcgtgtcagacaaagggcttagcttattgcagcc

SEQ ID NO: 484 - DNA, 36 bp, Artificial Sequence
  source: /note="Description of Artificial Sequence: Syntheticoligonucleotide"
attaatttgacacaatgcaatgatgggtcgcggtct

SEQ ID NO: 485 - DNA, 36 bp, Artificial Sequence
  source: /note="Description of Artificial Sequence: Syntheticoligonucleotide"
atcacaatagaattgacgccaacaagacgcgggtga

SEQ ID NO: 486 - DNA, 36 bp, Artificial Sequence
  source: /note="Description of Artificial Sequence: Syntheticoligonucleotide"
agatacaatatagagtacagccacagggatgcatgc

SEQ ID NO: 487 - DNA, 36 bp, Artificial Sequence
  source: /note="Description of Artificial Sequence: Syntheticoligonucleotide"
tgctttgcggcaacaacaatgcttctgccgctttac

SEQ ID NO: 488 - DNA, 36 bp, Artificial Sequence
  source: /note="Description of Artificial Sequence: Syntheticoligonucleotide"
ttcgtattaaggtttgggtcagtgaggcagtgagca

SEQ ID NO: 489 - DNA, 36 bp, Artificial Sequence
  source: /note="Description of Artificial Sequence: Syntheticoligonucleotide"
ctattaagagtacgatgtgcgtggcgcgacttcatg

SEQ ID NO: 490 - DNA, 36 bp, Artificial Sequence
  source: /note="Description of Artificial Sequence: Syntheticoligonucleotide"
ttggcgtaactatcaagcaatgcacaagttctcggt

SEQ ID NO: 491 - DNA, 36 bp, Artificial Sequence
  source: /note="Description of Artificial Sequence: Syntheticoligonucleotide"
aacccggtccgaggcaagaccctagaataacaaatt

SEQ ID NO: 492 - DNA, 36 bp, Artificial Sequence
  source: /note="Description of Artificial Sequence: Syntheticoligonucleotide"
acgaaggactgaatcacgttctacctctcacaagta

SEQ ID NO: 493 - DNA, 36 bp, Artificial Sequence
  source: /note="Description of Artificial Sequence: Syntheticoligonucleotide"
ggcacaggatatttcaccttagtgtcttagactccc

SEQ ID NO: 494 - DNA, 36 bp, Artificial Sequence
  source: /note="Description of Artificial Sequence: Syntheticoligonucleotide"
gcataaccaagaggacgacaagtgcatatactacag

SEQ ID NO: 495 - DNA, 36 bp, Artificial Sequence
  source: /note="Description of Artificial Sequence: Syntheticoligonucleotide"
acacgaggatctaagtgtttgggagaggtgtcatcg

SEQ ID NO: 496 - DNA, 36 bp, Artificial Sequence
  source: /note="Description of Artificial Sequence: Syntheticoligonucleotide"
acctaccaatacaaataacgagggatcgtaacgaca

SEQ ID NO: 497 - DNA, 36 bp, Artificial Sequence
  source: /note="Description of Artificial Sequence: Syntheticoligonucleotide"
gccaggctttactaggtttacctttctcttgcagag

SEQ ID NO: 498 - DNA, 36 bp, Artificial Sequence
  source: /note="Description of Artificial Sequence: Syntheticoligonucleotide"
gtgggtcatttgctcatacccggtgttgaagaccaa

SEQ ID NO: 499 - DNA, 36 bp, Artificial Sequence
  source: /note="Description of Artificial Sequence: Syntheticoligonucleotide"
gtttagcagcatgagacaccacctcaatgctaagcg

SEQ ID NO: 500 - DNA, 36 bp, Artificial Sequence
  source: /note="Description of Artificial Sequence: Syntheticoligonucleotide"
cagttagcatgactcgcttatattcccgatgctggc

SEQ ID NO: 501 - DNA, 36 bp, Artificial Sequence
  source: /note="Description of Artificial Sequence: Syntheticoligonucleotide"
gacaagactttgtagatttgccagcgttcgccttgg

SEQ ID NO: 502 - DNA, 36 bp, Artificial Sequence
  source: /note="Description of Artificial Sequence: Syntheticoligonucleotide"
ctgatagatccaaatgctgcatagtgcgcttgtcgg

SEQ ID NO: 503 - DNA, 36 bp, Artificial Sequence
  source: /note="Description of Artificial Sequence: Syntheticoligonucleotide"
gccagtatgtcctgcactgtttatgatctgtaccaa

SEQ ID NO: 504 - DNA, 36 bp, Artificial Sequence
  source: /note="Description of Artificial Sequence: Syntheticoligonucleotide"
aatccgtttggtgttcctgtccacaccgcttcatgt

SEQ ID NO: 505 - DNA, 36 bp, Artificial Sequence
  source: /note="Description of Artificial Sequence: Syntheticoligonucleotide"
atacggtatgtagatggccgagacttcctgggatca

SEQ ID NO: 506 - DNA, 36 bp, Artificial Sequence
  source: /note="Description of Artificial Sequence: Syntheticoligonucleotide"
atcgagccgatgtgtggcagggatatggtcacattt

SEQ ID NO: 507 - DNA, 36 bp, Artificial Sequence
  source: /note="Description of Artificial Sequence: Syntheticoligonucleotide"
tacgcgcagagcaggaggtttagtataaattgggtg

SEQ ID NO: 508 - DNA, 36 bp, Artificial Sequence
  source: /note="Description of Artificial Sequence: Syntheticoligonucleotide"
tcaaggcacctttgacgcttatcggcagtcttgaaa

SEQ ID NO: 509 - DNA, 36 bp, Artificial Sequence
  source: /note="Description of Artificial Sequence: Syntheticoligonucleotide"
ttctgctacggcgataagacgtgtgcttccgttaga

SEQ ID NO: 510 - DNA, 36 bp, Artificial Sequence
  source: /note="Description of Artificial Sequence: Syntheticoligonucleotide"
ttaagggcaattctgctcgggcatagcggaacctta

SEQ ID NO: 511 - DNA, 36 bp, Artificial Sequence
  source: /note="Description of Artificial Sequence: Syntheticoligonucleotide"
acacatctgttattgcagacagtccgctccgaatat

SEQ ID NO: 512 - DNA, 36 bp, Artificial Sequence
  source: /note="Description of Artificial Sequence: Syntheticoligonucleotide"
aagatatcgattcacatctacccgcaattcggtcgt

SEQ ID NO: 513 - DNA, 36 bp, Artificial Sequence
  source: /note="Description of Artificial Sequence: Syntheticoligonucleotide"
aactcgataaatgatacacgccaaatattggagggc

SEQ ID NO: 514 - DNA, 36 bp, Artificial Sequence
  source: /note="Description of Artificial Sequence: Syntheticoligonucleotide"
aatgtccataatcgacgacgagtgtccgcttgacta

SEQ ID NO: 515 - DNA, 36 bp, Artificial Sequence
  source: /note="Description of Artificial Sequence: Syntheticoligonucleotide"
cagacaagtagggtccatatgcatcaactatggaaa

SEQ ID NO: 516 - DNA, 36 bp, Artificial Sequence
  source: /note="Description of Artificial Sequence: Syntheticoligonucleotide"
gctgcaacaagtttgagtatctttgatagggacctc

SEQ ID NO: 517 - DNA, 36 bp, Artificial Sequence
  source: /note="Description of Artificial Sequence: Syntheticoligonucleotide"
taggaacccacgaatgtctgtcaagcagtttatctc

SEQ ID NO: 518 - DNA, 36 bp, Artificial Sequence
  source: /note="Description of Artificial Sequence: Syntheticoligonucleotide"
ctttcatgccggctctgtgaaaggaagtatattgag

SEQ ID NO: 519 - DNA, 36 bp, Artificial Sequence
  source: /note="Description of Artificial Sequence: Syntheticoligonucleotide"
gttctgtcgggatataaacacccatgtgctggtctg

SEQ ID NO: 520 - DNA, 36 bp, Artificial Sequence
  source: /note="Description of Artificial Sequence: Syntheticoligonucleotide"
ccggtttcatttctgaccaaagcgatagacctacaa

SEQ ID NO: 521 - DNA, 36 bp, Artificial Sequence
  source: /note="Description of Artificial Sequence: Syntheticoligonucleotide"
aacgtcaagttctttaccacctagctcgcgaaatca

SEQ ID NO: 522 - DNA, 36 bp, Artificial Sequence
  source: /note="Description of Artificial Sequence: Syntheticoligonucleotide"
cgttaggatactgcgggtcacaatctgatgtacagg

SEQ ID NO: 523 - DNA, 36 bp, Artificial Sequence
  source: /note="Description of Artificial Sequence: Syntheticoligonucleotide"
attgtcgtgtcaggtccaacttcgctcaagcttcta

SEQ ID NO: 524 - DNA, 36 bp, Artificial Sequence
  source: /note="Description of Artificial Sequence: Syntheticoligonucleotide"
ccgatgagaaacatcattagcgttaactcgtgccct

SEQ ID NO: 525 - DNA, 36 bp, Artificial Sequence
  source: /note="Description of Artificial Sequence: Syntheticoligonucleotide"
gtatccttcgtcaccgtttgaggccgctctttaact

SEQ ID NO: 526 - DNA, 36 bp, Artificial Sequence
  source: /note="Description of Artificial Sequence: Syntheticoligonucleotide"
cctatgggtgagaggtacgcaggtatcatatcacta

SEQ ID NO: 527 - DNA, 36 bp, Artificial Sequence
  source: /note="Description of Artificial Sequence: Syntheticoligonucleotide"
gtaagtcggttgcgataacgtagattagtttggcac

SEQ ID NO: 528 - DNA, 36 bp, Artificial Sequence
  source: /note="Description of Artificial Sequence: Syntheticoligonucleotide"
ttggagcgggtgagcctattacataagtctctcatc

SEQ ID NO: 529 - DNA, 36 bp, Artificial Sequence
  source: /note="Description of Artificial Sequence: Syntheticoligonucleotide"
atttccaatgcccaacgtgttagttgaatggtcgcc

SEQ ID NO: 530 - DNA, 36 bp, Artificial Sequence
  source: /note="Description of Artificial Sequence: Syntheticoligonucleotide"
gttctcacgtacgagaagaatacagcgaaataagga

SEQ ID NO: 531 - DNA, 36 bp, Artificial Sequence
  source: /note="Description of Artificial Sequence: Syntheticoligonucleotide"
ggacacccgatatacaagagtagtgaatcctcagtc

SEQ ID NO: 532 - DNA, 36 bp, Artificial Sequence
  source: /note="Description of Artificial Sequence: Syntheticoligonucleotide"
tatcgaattcacttccgcatcgtgattgcttgctaa

SEQ ID NO: 533 - DNA, 36 bp, Artificial Sequence
  source: /note="Description of Artificial Sequence: Syntheticoligonucleotide"
ccgctacactaatgtttaccagaggctatgtccaac

SEQ ID NO: 534 - DNA, 36 bp, Artificial Sequence
  source: /note="Description of Artificial Sequence: Syntheticoligonucleotide"
cgagatgaggtcgacttagaataacgtttgcaacta

SEQ ID NO: 535 - DNA, 36 bp, Artificial Sequence
  source: /note="Description of Artificial Sequence: Syntheticoligonucleotide"
aggaagcttagattactgggtaatggcctcgtatca

SEQ ID NO: 536 - DNA, 36 bp, Artificial Sequence
  source: /note="Description of Artificial Sequence: Syntheticoligonucleotide"
gctgcaattatcacctgtttcgtaagcagctacatc

SEQ ID NO: 537 - DNA, 36 bp, Artificial Sequence
  source: /note="Description of Artificial Sequence: Syntheticoligonucleotide"
tgagggctttcgtttagtcaatagtgtggtgctatt

SEQ ID NO: 538 - DNA, 36 bp, Artificial Sequence
  source: /note="Description of Artificial Sequence: Syntheticoligonucleotide"
ttcttgatgggttgggctatctcagtgacatctagg

SEQ ID NO: 539 - DNA, 36 bp, Artificial Sequence
  source: /note="Description of Artificial Sequence: Syntheticoligonucleotide"
gaatgttgatacaggatgtcggtaggccagtcctta

SEQ ID NO: 540 - DNA, 36 bp, Artificial Sequence
  source: /note="Description of Artificial Sequence: Syntheticoligonucleotide"
acctaaacaggacaatcgcatggagtcgcagtcgta

SEQ ID NO: 541 - DNA, 36 bp, Artificial Sequence
  source: /note="Description of Artificial Sequence: Syntheticoligonucleotide"
gcctccttactgaattgtctataccggcgattaaca

SEQ ID NO: 542 - DNA, 36 bp, Artificial Sequence
  source: /note="Description of Artificial Sequence: Syntheticoligonucleotide"
gtagtcctaggttgatgaggctcctaagaaatacgt

SEQ ID NO: 543 - DNA, 36 bp, Artificial Sequence
  source: /note="Description of Artificial Sequence: Syntheticoligonucleotide"
atcaattcgaagggcgttacaaattgaccggacagg

SEQ ID NO: 544 - DNA, 36 bp, Artificial Sequence
  source: /note="Description of Artificial Sequence: Syntheticoligonucleotide"
tcactctagataatccctaattggtggcccgcgttt

SEQ ID NO: 545 - DNA, 36 bp, Artificial Sequence
  source: /note="Description of Artificial Sequence: Syntheticoligonucleotide"
gtctaatggttgcggtcgttaaacgtctgtggttgg

SEQ ID NO: 546 - DNA, 36 bp, Artificial Sequence
  source: /note="Description of Artificial Sequence: Syntheticoligonucleotide"
cgccttaagtgtataactgtcggcctctatgtcgca

SEQ ID NO: 547 - DNA, 36 bp, Artificial Sequence
  source: /note="Description of Artificial Sequence: Syntheticoligonucleotide"
atctcattctctcgtggctgacgtctgatttcacct

SEQ ID NO: 548 - DNA, 36 bp, Artificial Sequence
  source: /note="Description of Artificial Sequence: Syntheticoligonucleotide"
attacgagccgcctagttacccttcggaattaatga

SEQ ID NO: 549 - DNA, 36 bp, Artificial Sequence
  source: /note="Description of Artificial Sequence: Syntheticoligonucleotide"
tggattccagacgacacgatggcttccatataagac

SEQ ID NO: 550 - DNA, 36 bp, Artificial Sequence
  source: /note="Description of Artificial Sequence: Syntheticoligonucleotide"
attgggcaaactttcgcgctactagttgtcaaagcc

SEQ ID NO: 551 - DNA, 36 bp, Artificial Sequence
  source: /note="Description of Artificial Sequence: Syntheticoligonucleotide"
cttaaacgatcgagggtctgttctagcggctcagta

SEQ ID NO: 552 - DNA, 36 bp, Artificial Sequence
  source: /note="Description of Artificial Sequence: Syntheticoligonucleotide"
aatctcccggtatggttgtgaaattgcatcactggg

SEQ ID NO: 553 - DNA, 36 bp, Artificial Sequence
  source: /note="Description of Artificial Sequence: Syntheticoligonucleotide"
gctagactaagccgtgcctaggtattgccacaactt

SEQ ID NO: 554 - DNA, 36 bp, Artificial Sequence
  source: /note="Description of Artificial Sequence: Syntheticoligonucleotide"
gtcctacaccttcacggctatgaggtatgtctgact

SEQ ID NO: 555 - DNA, 36 bp, Artificial Sequence
  source: /note="Description of Artificial Sequence: Syntheticoligonucleotide"
ggattaacgtgctctaggttcgatgataacgggcgt

SEQ ID NO: 556 - DNA, 36 bp, Artificial Sequence
  source: /note="Description of Artificial Sequence: Syntheticoligonucleotide"
cagaggtcctttacactcgctgccatttggccttaa

SEQ ID NO: 557 - DNA, 36 bp, Artificial Sequence
  source: /note="Description of Artificial Sequence: Syntheticoligonucleotide"
agggcatcgtaggcggattaaagcgtgttgtgttga

SEQ ID NO: 558 - DNA, 36 bp, Artificial Sequence
  source: /note="Description of Artificial Sequence: Syntheticoligonucleotide"
tagctctgtagtaattgtgtgagtcggtccgttgca

SEQ ID NO: 559 - DNA, 36 bp, Artificial Sequence
  source: /note="Description of Artificial Sequence: Syntheticoligonucleotide"
tggctagtacagacccaatggtcaatcagcacagag

SEQ ID NO: 560 - DNA, 36 bp, Artificial Sequence
  source: /note="Description of Artificial Sequence: Syntheticoligonucleotide"
agcagaacttggttactggtttacgttcgggaggta

SEQ ID NO: 561 - DNA, 36 bp, Artificial Sequence
  source: /note="Description of Artificial Sequence: Syntheticoligonucleotide"
tatgctcccgtgtcctcgataatcacatcgctttcc

SEQ ID NO: 562 - DNA, 36 bp, Artificial Sequence
  source: /note="Description of Artificial Sequence: Syntheticoligonucleotide"
agtggtggactaaagacaacggtggcgtagtcaaat

SEQ ID NO: 563 - DNA, 36 bp, Artificial Sequence
  source: /note="Description of Artificial Sequence: Syntheticoligonucleotide"
aaaggtatggacgtcgccgtagataagtactgtaca

SEQ ID NO: 564 - DNA, 36 bp, Artificial Sequence
  source: /note="Description of Artificial Sequence: Syntheticoligonucleotide"
ggtaattcgagtgggtgtccctcaacctcagatttg

SEQ ID NO: 565 - DNA, 36 bp, Artificial Sequence
  source: /note="Description of Artificial Sequence: Syntheticoligonucleotide"
caatcttcctggtgttgcctggtctctcacgtatat

SEQ ID NO: 566 - DNA, 36 bp, Artificial Sequence
  source: /note="Description of Artificial Sequence: Syntheticoligonucleotide"
ttaagagtccgatcagctgcctaaatggcagtgagc

SEQ ID NO: 567 - DNA, 36 bp, Artificial Sequence
  source: /note="Description of Artificial Sequence: Syntheticoligonucleotide"
ggatgtttggaaatcgtcaggagacaaccagagacg

SEQ ID NO: 568 - DNA, 36 bp, Artificial Sequence
  source: /note="Description of Artificial Sequence: Syntheticoligonucleotide"
ttccagaggtctaagttaccatgctggtaggctgag

SEQ ID NO: 569 - DNA, 36 bp, Artificial Sequence
  source: /note="Description of Artificial Sequence: Syntheticoligonucleotide"
ttgacagacaagagacactccttaagaggaaggctc

SEQ ID NO: 570 - DNA, 36 bp, Artificial Sequence
  source: /note="Description of Artificial Sequence: Syntheticoligonucleotide"
agtgcattggtaacagcaaattacagagggtcgcaa

SEQ ID NO: 571 - DNA, 36 bp, Artificial Sequence
  source: /note="Description of Artificial Sequence: Syntheticoligonucleotide"
gatacggtctactacgatagggctgctacttgccaa

SEQ ID NO: 572 - DNA, 36 bp, Artificial Sequence
  source: /note="Description of Artificial Sequence: Syntheticoligonucleotide"
agggatgcagtgttgatggataagctcacaccagaa

SEQ ID NO: 573 - DNA, 36 bp, Artificial Sequence
  source: /note="Description of Artificial Sequence: Syntheticoligonucleotide"
cacgatagatcgattggttcgcccgagaaatatccg

SEQ ID NO: 574 - DNA, 36 bp, Artificial Sequence
  source: /note="Description of Artificial Sequence: Syntheticoligonucleotide"
tatgattcccaatgacggtatcacggctgcgcatct

SEQ ID NO: 575 - DNA, 36 bp, Artificial Sequence
  source: /note="Description of Artificial Sequence: Syntheticoligonucleotide"
tccacaaagaaagatctgtggttcataggtgtccgt

SEQ ID NO: 576 - DNA, 36 bp, Artificial Sequence
  source: /note="Description of Artificial Sequence: Syntheticoligonucleotide"
gctctgcctcgttgcaaacgaaagtcaattagcctc

SEQ ID NO: 577 - DNA, 36 bp, Artificial Sequence
  source: /note="Description of Artificial Sequence: Syntheticoligonucleotide"
ctgccttagaatgtatttcacaaacctctaccatcc

SEQ ID NO: 578 - DNA, 36 bp, Artificial Sequence
  source: /note="Description of Artificial Sequence: Syntheticoligonucleotide"
gtcatggcactaaattcagcccatctgctacgccta

SEQ ID NO: 579 - DNA, 36 bp, Artificial Sequence
  source: /note="Description of Artificial Sequence: Syntheticoligonucleotide"
cagagatgcaatatggcctaaacccataagagactc

SEQ ID NO: 580 - DNA, 36 bp, Artificial Sequence
  source: /note="Description of Artificial Sequence: Syntheticoligonucleotide"
gaaagaaactctgacgccctttcgccaagccattga

SEQ ID NO: 581 - DNA, 36 bp, Artificial Sequence
  source: /note="Description of Artificial Sequence: Syntheticoligonucleotide"
catgaatgtgccgaaccgagtcacgggcaaagtata

SEQ ID NO: 582 - DNA, 36 bp, Artificial Sequence
  source: /note="Description of Artificial Sequence: Syntheticoligonucleotide"
gagccacaagacatccatttattgctcattagcgtg

SEQ ID NO: 583 - DNA, 36 bp, Artificial Sequence
  source: /note="Description of Artificial Sequence: Syntheticoligonucleotide"
cattgtagggttcgcttggaccggaaatcatgggtt

SEQ ID NO: 584 - DNA, 36 bp, Artificial Sequence
  source: /note="Description of Artificial Sequence: Syntheticoligonucleotide"
cccatcacgacgttgtcagcgcttaatatttatgcc

SEQ ID NO: 585 - DNA, 36 bp, Artificial Sequence
  source: /note="Description of Artificial Sequence: Syntheticoligonucleotide"
tcagtcagtaaatagtgtgtgtatgtgtcactcgag

SEQ ID NO: 586 - DNA, 36 bp, Artificial Sequence
  source: /note="Description of Artificial Sequence: Syntheticoligonucleotide"
aagtttctacgagtagtgggaagcgtagagcctggt

SEQ ID NO: 587 - DNA, 36 bp, Artificial Sequence
  source: /note="Description of Artificial Sequence: Syntheticoligonucleotide"
tatcagagctcacgcggacagggagaatgtcaaaga

SEQ ID NO: 588 - DNA, 36 bp, Artificial Sequence
  source: /note="Description of Artificial Sequence: Syntheticoligonucleotide"
aggatgcgctagatggacataatgaaatgaccgttg

SEQ ID NO: 589 - DNA, 36 bp, Artificial Sequence
  source: /note="Description of Artificial Sequence: Syntheticoligonucleotide"
aagacgccttcgactaggcagtgtgttaggaagaac

SEQ ID NO: 590 - DNA, 36 bp, Artificial Sequence
  source: /note="Description of Artificial Sequence: Syntheticoligonucleotide"
accaaggcggcgaacttatgttggttatgaatagcc

SEQ ID NO: 591 - DNA, 36 bp, Artificial Sequence
  source: /note="Description of Artificial Sequence: Syntheticoligonucleotide"
cactcgcaaatgctcgtcatgcgaagttgagttaag

SEQ ID NO: 592 - DNA, 36 bp, Artificial Sequence
  source: /note="Description of Artificial Sequence: Syntheticoligonucleotide"
gtcccacaataacaaccttgcggtttagcagacttg

SEQ ID NO: 593 - DNA, 36 bp, Artificial Sequence
  source: /note="Description of Artificial Sequence: Syntheticoligonucleotide"
gcgagtttacttgaaagttgtagcttaggaacgacc

SEQ ID NO: 594 - DNA, 36 bp, Artificial Sequence
  source: /note="Description of Artificial Sequence: Syntheticoligonucleotide"
accataacccgtctacgaagaatccttaacacgtca

SEQ ID NO: 595 - DNA, 36 bp, Artificial Sequence
  source: /note="Description of Artificial Sequence: Syntheticoligonucleotide"
tagaacggtacgtgctttaaatgctaatccagtcgc

SEQ ID NO: 596 - DNA, 36 bp, Artificial Sequence
  source: /note="Description of Artificial Sequence: Syntheticoligonucleotide"
atatgatgaatttgtccttgggcgagaggcgtcctg

SEQ ID NO: 597 - DNA, 36 bp, Artificial Sequence
  source: /note="Description of Artificial Sequence: Syntheticoligonucleotide"
catctagggatgtaagtctttacgcagacaactgcc

SEQ ID NO: 598 - DNA, 36 bp, Artificial Sequence
  source: /note="Description of Artificial Sequence: Syntheticoligonucleotide"
cacacgattcacgcgcttatattatcgttcatcaac

SEQ ID NO: 599 - DNA, 36 bp, Artificial Sequence
  source: /note="Description of Artificial Sequence: Syntheticoligonucleotide"
gcgttcgtctattactcaatcaatccgtgtgtagta

SEQ ID NO: 600 - DNA, 36 bp, Artificial Sequence
  source: /note="Description of Artificial Sequence: Syntheticoligonucleotide"
tcatagtgcgaggatccgaacattgtctaaccgatc

SEQ ID NO: 601 - DNA, 36 bp, Artificial Sequence
  source: /note="Description of Artificial Sequence: Syntheticoligonucleotide"
ggtatgttaagcagttagatacactgtgacaggtgg

SEQ ID NO: 602 - DNA, 36 bp, Artificial Sequence
  source: /note="Description of Artificial Sequence: Syntheticoligonucleotide"
tgtactgcccaacgagttgtattctgcaatgagtcg

SEQ ID NO: 603 - DNA, 36 bp, Artificial Sequence
  source: /note="Description of Artificial Sequence: Syntheticoligonucleotide"
gccgccaattatttgcattgctgtactcacagggac

SEQ ID NO: 604 - DNA, 36 bp, Artificial Sequence
  source: /note="Description of Artificial Sequence: Syntheticoligonucleotide"
aaagttcataaaggctgtaaggacggatcaaggcgc

SEQ ID NO: 605 - DNA, 36 bp, Artificial Sequence
  source: /note="Description of Artificial Sequence: Syntheticoligonucleotide"
aagcattgatctgatcgcatgcagcgatatccagaa

SEQ ID NO: 606 - DNA, 36 bp, Artificial Sequence
  source: /note="Description of Artificial Sequence: Syntheticoligonucleotide"
tcaccttaagccacggcgcatatttagttgactgac

SEQ ID NO: 607 - DNA, 36 bp, Artificial Sequence
  source: /note="Description of Artificial Sequence: Syntheticoligonucleotide"
aaacggagtaggaatgtgttctgtggaagaggatgt

SEQ ID NO: 608 - DNA, 36 bp, Artificial Sequence
  source: /note="Description of Artificial Sequence: Syntheticoligonucleotide"
ctatagttgttgggtgggacggaatagaagcacata

SEQ ID NO: 609 - DNA, 36 bp, Artificial Sequence
  source: /note="Description of Artificial Sequence: Syntheticoligonucleotide"
ggtctatatcaatttaatcgacgcgcacatgtatgc

SEQ ID NO: 610 - DNA, 36 bp, Artificial Sequence
  source: /note="Description of Artificial Sequence: Syntheticoligonucleotide"
acttataatgggcagggcgctggaagttactcagca

SEQ ID NO: 611 - DNA, 36 bp, Artificial Sequence
  source: /note="Description of Artificial Sequence: Syntheticoligonucleotide"
agtagttaaccctgtacaataaggaggcattcgaac

SEQ ID NO: 612 - DNA, 36 bp, Artificial Sequence
  source: /note="Description of Artificial Sequence: Syntheticoligonucleotide"
aatatcttgtttcagttcccggcacgcattaagccc

SEQ ID NO: 613 - DNA, 36 bp, Artificial Sequence
  source: /note="Description of Artificial Sequence: Syntheticoligonucleotide"
cggagtcatccgggattcaattgtagtcgatacatg

SEQ ID NO: 614 - DNA, 36 bp, Artificial Sequence
  source: /note="Description of Artificial Sequence: Syntheticoligonucleotide"
tcgtccggttacctcaccattaagtagaaccggagt

SEQ ID NO: 615 - DNA, 36 bp, Artificial Sequence
  source: /note="Description of Artificial Sequence: Syntheticoligonucleotide"
attcacagccgagtatagatttcgtctgcctgtatt

SEQ ID NO: 616 - DNA, 36 bp, Artificial Sequence
  source: /note="Description of Artificial Sequence: Syntheticoligonucleotide"
gtaactatactactaggagctcggccagcttgaatg

SEQ ID NO: 617 - DNA, 36 bp, Artificial Sequence
  source: /note="Description of Artificial Sequence: Syntheticoligonucleotide"
ccttcatgaccgcagaaagctgcgaatagtttgatg

SEQ ID NO: 618 - DNA, 36 bp, Artificial Sequence
  source: /note="Description of Artificial Sequence: Syntheticoligonucleotide"
ttatatgctctttgggtattctatcgcgcgtaagca

SEQ ID NO: 619 - DNA, 36 bp, Artificial Sequence
  source: /note="Description of Artificial Sequence: Syntheticoligonucleotide"
ttgtcgatgatggacccagaattacgcctccaacct

SEQ ID NO: 620 - DNA, 36 bp, Artificial Sequence
  source: /note="Description of Artificial Sequence: Syntheticoligonucleotide"
gggtgactagattgtactccggccaccctaaacttt

SEQ ID NO: 621 - DNA, 36 bp, Artificial Sequence
  source: /note="Description of Artificial Sequence: Syntheticoligonucleotide"
ggctggagcatgattggcgacgggactatttcttaa

SEQ ID NO: 622 - DNA, 36 bp, Artificial Sequence
  source: /note="Description of Artificial Sequence: Syntheticoligonucleotide"
gcaagcactgtttctaggcccggagagatagtatca

SEQ ID NO: 623 - DNA, 36 bp, Artificial Sequence
  source: /note="Description of Artificial Sequence: Syntheticoligonucleotide"
cagcagcaagtctcatgcatagtttccttactagtt

SEQ ID NO: 624 - DNA, 36 bp, Artificial Sequence
  source: /note="Description of Artificial Sequence: Syntheticoligonucleotide"
ttgtcgtgccggaggtctaggagacagaaacgtatt

SEQ ID NO: 625 - DNA, 36 bp, Artificial Sequence
  source: /note="Description of Artificial Sequence: Syntheticoligonucleotide"
aatgactttgcgactcagaaaggcgaatcgatgacc

SEQ ID NO: 626 - DNA, 36 bp, Artificial Sequence
  source: /note="Description of Artificial Sequence: Syntheticoligonucleotide"
taagaagcttgctcggtaagttaaccatatgcctca

SEQ ID NO: 627 - DNA, 36 bp, Artificial Sequence
  source: /note="Description of Artificial Sequence: Syntheticoligonucleotide"
aatctctcgcgcacgtgataaacatccatccatacg

SEQ ID NO: 628 - DNA, 36 bp, Artificial Sequence
  source: /note="Description of Artificial Sequence: Syntheticoligonucleotide"
tagaccagaactctaatttatcgaaacggccaacac

SEQ ID NO: 629 - DNA, 36 bp, Artificial Sequence
  source: /note="Description of Artificial Sequence: Syntheticoligonucleotide"
atcgaattagacaagcgtgtttcatgacgagcaggg

SEQ ID NO: 630 - DNA, 36 bp, Artificial Sequence
  source: /note="Description of Artificial Sequence: Syntheticoligonucleotide"
ggcgttaaagagttaatgctgatttcgagagcgccg

SEQ ID NO: 631 - DNA, 36 bp, Artificial Sequence
  source: /note="Description of Artificial Sequence: Syntheticoligonucleotide"
ctggcgtgatataaagtcgagagtaacccgtaacgt

SEQ ID NO: 632 - DNA, 36 bp, Artificial Sequence
  source: /note="Description of Artificial Sequence: Syntheticoligonucleotide"
tcgccgaacaaatctgagataaggactcctggactg

SEQ ID NO: 633 - DNA, 36 bp, Artificial Sequence
  source: /note="Description of Artificial Sequence: Syntheticoligonucleotide"
gtatggttgctgtcaggttagtgaaaggaagtgagg

SEQ ID NO: 634 - DNA, 36 bp, Artificial Sequence
  source: /note="Description of Artificial Sequence: Syntheticoligonucleotide"
atatagctttagtggcgttgctgagcgaaagagtaa

SEQ ID NO: 635 - DNA, 36 bp, Artificial Sequence
  source: /note="Description of Artificial Sequence: Syntheticoligonucleotide"
tacaaatttctttagggcctgccgatacagttcgcc

SEQ ID NO: 636 - DNA, 36 bp, Artificial Sequence
  source: /note="Description of Artificial Sequence: Syntheticoligonucleotide"
gtcgatatgatagtacacaactagcctctggcgact

SEQ ID NO: 637 - DNA, 36 bp, Artificial Sequence
  source: /note="Description of Artificial Sequence: Syntheticoligonucleotide"
ttattgtcgcaccatatagtaggctgcacagataga

SEQ ID NO: 638 - DNA, 36 bp, Artificial Sequence
  source: /note="Description of Artificial Sequence: Syntheticoligonucleotide"
catacatcttccagttgacgtcgctcgcttaaggaa

SEQ ID NO: 639 - DNA, 36 bp, Artificial Sequence
  source: /note="Description of Artificial Sequence: Syntheticoligonucleotide"
agcgataaactgaacatatccagggaggtgatccat

SEQ ID NO: 640 - DNA, 36 bp, Artificial Sequence
  source: /note="Description of Artificial Sequence: Syntheticoligonucleotide"
tttcacccaaccatgggctaagaatagtttgagatg

SEQ ID NO: 641 - DNA, 36 bp, Artificial Sequence
  source: /note="Description of Artificial Sequence: Syntheticoligonucleotide"
ctgcgtaatcaacagatcttggtagtagttggacag

SEQ ID NO: 642 - DNA, 36 bp, Artificial Sequence
  source: /note="Description of Artificial Sequence: Syntheticoligonucleotide"
tgataattgtggtagtcccagttacggtaccttcca

SEQ ID NO: 643 - DNA, 36 bp, Artificial Sequence
  source: /note="Description of Artificial Sequence: Syntheticoligonucleotide"
atgctcaatccaggtacacttgcgtatccgacgctt

SEQ ID NO: 644 - DNA, 36 bp, Artificial Sequence
  source: /note="Description of Artificial Sequence: Syntheticoligonucleotide"
atcagttccagaaagtggacaacggcgacatatata

SEQ ID NO: 645 - DNA, 36 bp, Artificial Sequence
  source: /note="Description of Artificial Sequence: Syntheticoligonucleotide"
tataataatcgtgcgggctcataacgaatccggctg

SEQ ID NO: 646 - DNA, 36 bp, Artificial Sequence
  source: /note="Description of Artificial Sequence: Syntheticoligonucleotide"
tacttagatgcaccgcaacacagcccatagcagcta

SEQ ID NO: 647 - DNA, 36 bp, Artificial Sequence
  source: /note="Description of Artificial Sequence: Syntheticoligonucleotide"
tcaatgagtggcacctgcttatttgtaacgatcggg

SEQ ID NO: 648 - DNA, 36 bp, Artificial Sequence
  source: /note="Description of Artificial Sequence: Syntheticoligonucleotide"
gggagcgctttgaacttcgatgtctgtacacgtact

SEQ ID NO: 649 - DNA, 36 bp, Artificial Sequence
  source: /note="Description of Artificial Sequence: Syntheticoligonucleotide"
ctgactaaggagatgaatgacagacctacctgtcca

SEQ ID NO: 650 - DNA, 36 bp, Artificial Sequence
  source: /note="Description of Artificial Sequence: Syntheticoligonucleotide"
tgttagtccgtcctactgtggttagaccgatctgag

SEQ ID NO: 651 - DNA, 36 bp, Artificial Sequence
  source: /note="Description of Artificial Sequence: Syntheticoligonucleotide"
acctcgcattcggaacctcggaactagatcttactt

SEQ ID NO: 652 - DNA, 36 bp, Artificial Sequence
  source: /note="Description of Artificial Sequence: Syntheticoligonucleotide"
gcttcgcagaattggaatcttgaaaggtcactcacc

SEQ ID NO: 653 - DNA, 36 bp, Artificial Sequence
  source: /note="Description of Artificial Sequence: Syntheticoligonucleotide"
gcttgtaagtcccaagaccaagcagtcagcggattt

SEQ ID NO: 654 - DNA, 36 bp, Artificial Sequence
  source: /note="Description of Artificial Sequence: Syntheticoligonucleotide"
aggagttagcgagatgtaggctcctcgttctatgtt

SEQ ID NO: 655 - DNA, 36 bp, Artificial Sequence
  source: /note="Description of Artificial Sequence: Syntheticoligonucleotide"
acacaaccctgccattactcctgtgttagctagtca

SEQ ID NO: 656 - DNA, 36 bp, Artificial Sequence
  source: /note="Description of Artificial Sequence: Syntheticoligonucleotide"
tcgaatgagtttgttcaatcgtgacttccgatgtgt

SEQ ID NO: 657 - DNA, 36 bp, Artificial Sequence
  source: /note="Description of Artificial Sequence: Syntheticoligonucleotide"
agcttaacgtcgtggctggaacactcaggatttgta

SEQ ID NO: 658 - DNA, 36 bp, Artificial Sequence
  source: /note="Description of Artificial Sequence: Syntheticoligonucleotide"
tcgcttcactctctcttaccactaggcacactaata

SEQ ID NO: 659 - DNA, 36 bp, Artificial Sequence
  source: /note="Description of Artificial Sequence: Syntheticoligonucleotide"
acctggccttcccaattagcatacgccgtacttgtt

SEQ ID NO: 660 - DNA, 36 bp, Artificial Sequence
  source: /note="Description of Artificial Sequence: Syntheticoligonucleotide"
ttatccctgagattgagttagagcactgtcgtatgt

SEQ ID NO: 661 - DNA, 36 bp, Artificial Sequence
  source: /note="Description of Artificial Sequence: Syntheticoligonucleotide"
tccgagcagtttaccagtttggaagtgtttgttgag

SEQ ID NO: 662 - DNA, 36 bp, Artificial Sequence
  source: /note="Description of Artificial Sequence: Syntheticoligonucleotide"
cctggcaagatgagctgcttgaatgtgaaccagcat

SEQ ID NO: 663 - DNA, 36 bp, Artificial Sequence
  source: /note="Description of Artificial Sequence: Syntheticoligonucleotide"
acgctgtcaacgatgtacaggagaatggtgggaagt

SEQ ID NO: 664 - DNA, 36 bp, Artificial Sequence
  source: /note="Description of Artificial Sequence: Syntheticoligonucleotide"
cggcacttagcacgaaacctacgagttatatgtgat

SEQ ID NO: 665 - DNA, 36 bp, Artificial Sequence
  source: /note="Description of Artificial Sequence: Syntheticoligonucleotide"
gatagacgatgagtctctttgctccaagtcctagag

SEQ ID NO: 666 - DNA, 36 bp, Artificial Sequence
  source: /note="Description of Artificial Sequence: Syntheticoligonucleotide"
ctaggaccctaataactacaccgtgggtttgcgttc

SEQ ID NO: 667 - DNA, 36 bp, Artificial Sequence
  source: /note="Description of Artificial Sequence: Syntheticoligonucleotide"
cgccttggtgtcgaaagaatccaacgtagttaaatc

SEQ ID NO: 668 - DNA, 36 bp, Artificial Sequence
  source: /note="Description of Artificial Sequence: Syntheticoligonucleotide"
tagacacgccatctacgtgcagtaatcagatgatat

SEQ ID NO: 669 - DNA, 36 bp, Artificial Sequence
  source: /note="Description of Artificial Sequence: Syntheticoligonucleotide"
ggctgcaataagctaagccctttgtctgacacgaag

SEQ ID NO: 670 - DNA, 36 bp, Artificial Sequence
  source: /note="Description of Artificial Sequence: Syntheticoligonucleotide"
agaccgcgacccatcattgcattgtgtcaaattaat

SEQ ID NO: 671 - DNA, 36 bp, Artificial Sequence
  source: /note="Description of Artificial Sequence: Syntheticoligonucleotide"
tcacccgcgtcttgttggcgtcaattctattgtgat

SEQ ID NO: 672 - DNA, 36 bp, Artificial Sequence
  source: /note="Description of Artificial Sequence: Syntheticoligonucleotide"
gcatgcatccctgtggctgtactctatattgtatct

SEQ ID NO: 673 - DNA, 36 bp, Artificial Sequence
  source: /note="Description of Artificial Sequence: Syntheticoligonucleotide"
gtaaagcggcagaagcattgttgttgccgcaaagca

SEQ ID NO: 674 - DNA, 36 bp, Artificial Sequence
  source: /note="Description of Artificial Sequence: Syntheticoligonucleotide"
tgctcactgcctcactgacccaaaccttaatacgaa

SEQ ID NO: 675 - DNA, 36 bp, Artificial Sequence
  source: /note="Description of Artificial Sequence: Syntheticoligonucleotide"
catgaagtcgcgccacgcacatcgtactcttaatag

SEQ ID NO: 676 - DNA, 36 bp, Artificial Sequence
  source: /note="Description of Artificial Sequence: Syntheticoligonucleotide"
accgagaacttgtgcattgcttgatagttacgccaa

SEQ ID NO: 677 - DNA, 36 bp, Artificial Sequence
  source: /note="Description of Artificial Sequence: Syntheticoligonucleotide"
aatttgttattctagggtcttgcctcggaccgggtt

SEQ ID NO: 678 - DNA, 36 bp, Artificial Sequence
  source: /note="Description of Artificial Sequence: Syntheticoligonucleotide"
tacttgtgagaggtagaacgtgattcagtccttcgt

SEQ ID NO: 679 - DNA, 36 bp, Artificial Sequence
  source: /note="Description of Artificial Sequence: Syntheticoligonucleotide"
gggagtctaagacactaaggtgaaatatcctgtgcc

SEQ ID NO: 680 - DNA, 36 bp, Artificial Sequence
  source: /note="Description of Artificial Sequence: Syntheticoligonucleotide"
ctgtagtatatgcacttgtcgtcctcttggttatgc

SEQ ID NO: 681 - DNA, 36 bp, Artificial Sequence
  source: /note="Description of Artificial Sequence: Syntheticoligonucleotide"
cgatgacacctctcccaaacacttagatcctcgtgt

SEQ ID NO: 682 - DNA, 36 bp, Artificial Sequence
  source: /note="Description of Artificial Sequence: Syntheticoligonucleotide"
tgtcgttacgatccctcgttatttgtattggtaggt

SEQ ID NO: 683 - DNA, 36 bp, Artificial Sequence
  source: /note="Description of Artificial Sequence: Syntheticoligonucleotide"
ctctgcaagagaaaggtaaacctagtaaagcctggc

SEQ ID NO: 684 - DNA, 36 bp, Artificial Sequence
  source: /note="Description of Artificial Sequence: Syntheticoligonucleotide"
ttggtcttcaacaccgggtatgagcaaatgacccac

SEQ ID NO: 685 - DNA, 36 bp, Artificial Sequence
  source: /note="Description of Artificial Sequence: Syntheticoligonucleotide"
cgcttagcattgaggtggtgtctcatgctgctaaac

SEQ ID NO: 686 - DNA, 36 bp, Artificial Sequence
  source: /note="Description of Artificial Sequence: Syntheticoligonucleotide"
gccagcatcgggaatataagcgagtcatgctaactg

SEQ ID NO: 687 - DNA, 36 bp, Artificial Sequence
  source: /note="Description of Artificial Sequence: Syntheticoligonucleotide"
ccaaggcgaacgctggcaaatctacaaagtcttgtc

SEQ ID NO: 688 - DNA, 36 bp, Artificial Sequence
  source: /note="Description of Artificial Sequence: Syntheticoligonucleotide"
ccgacaagcgcactatgcagcatttggatctatcag

SEQ ID NO: 689 - DNA, 36 bp, Artificial Sequence
  source: /note="Description of Artificial Sequence: Syntheticoligonucleotide"
ttggtacagatcataaacagtgcaggacatactggc

SEQ ID NO: 690 - DNA, 36 bp, Artificial Sequence
  source: /note="Description of Artificial Sequence: Syntheticoligonucleotide"
acatgaagcggtgtggacaggaacaccaaacggatt

SEQ ID NO: 691 - DNA, 36 bp, Artificial Sequence
  source: /note="Description of Artificial Sequence: Syntheticoligonucleotide"
tgatcccaggaagtctcggccatctacataccgtat

SEQ ID NO: 692 - DNA, 36 bp, Artificial Sequence
  source: /note="Description of Artificial Sequence: Syntheticoligonucleotide"
aaatgtgaccatatccctgccacacatcggctcgat

SEQ ID NO: 693 - DNA, 36 bp, Artificial Sequence
  source: /note="Description of Artificial Sequence: Syntheticoligonucleotide"
cacccaatttatactaaacctcctgctctgcgcgta

SEQ ID NO: 694 - DNA, 36 bp, Artificial Sequence
  source: /note="Description of Artificial Sequence: Syntheticoligonucleotide"
tttcaagactgccgataagcgtcaaaggtgccttga

SEQ ID NO: 695 - DNA, 36 bp, Artificial Sequence
  source: /note="Description of Artificial Sequence: Syntheticoligonucleotide"
tctaacggaagcacacgtcttatcgccgtagcagaa

SEQ ID NO: 696 - DNA, 36 bp, Artificial Sequence
  source: /note="Description of Artificial Sequence: Syntheticoligonucleotide"
taaggttccgctatgcccgagcagaattgcccttaa

SEQ ID NO: 697 - DNA, 36 bp, Artificial Sequence
  source: /note="Description of Artificial Sequence: Syntheticoligonucleotide"
atattcggagcggactgtctgcaataacagatgtgt

SEQ ID NO: 698 - DNA, 36 bp, Artificial Sequence
  source: /note="Description of Artificial Sequence: Syntheticoligonucleotide"
acgaccgaattgcgggtagatgtgaatcgatatctt

SEQ ID NO: 699 - DNA, 36 bp, Artificial Sequence
  source: /note="Description of Artificial Sequence: Syntheticoligonucleotide"
gccctccaatatttggcgtgtatcatttatcgagtt

SEQ ID NO: 700 - DNA, 36 bp, Artificial Sequence
  source: /note="Description of Artificial Sequence: Syntheticoligonucleotide"
tagtcaagcggacactcgtcgtcgattatggacatt

SEQ ID NO: 701 - DNA, 36 bp, Artificial Sequence
  source: /note="Description of Artificial Sequence: Syntheticoligonucleotide"
tttccatagttgatgcatatggaccctacttgtctg

SEQ ID NO: 702 - DNA, 36 bp, Artificial Sequence
  source: /note="Description of Artificial Sequence: Syntheticoligonucleotide"
gaggtccctatcaaagatactcaaacttgttgcagc

SEQ ID NO: 703 - DNA, 36 bp, Artificial Sequence
  source: /note="Description of Artificial Sequence: Syntheticoligonucleotide"
gagataaactgcttgacagacattcgtgggttccta

SEQ ID NO: 704 - DNA, 36 bp, Artificial Sequence
  source: /note="Description of Artificial Sequence: Syntheticoligonucleotide"
ctcaatatacttcctttcacagagccggcatgaaag

SEQ ID NO: 705 - DNA, 36 bp, Artificial Sequence
  source: /note="Description of Artificial Sequence: Syntheticoligonucleotide"
cagaccagcacatgggtgtttatatcccgacagaac

SEQ ID NO: 706 - DNA, 36 bp, Artificial Sequence
  source: /note="Description of Artificial Sequence: Syntheticoligonucleotide"
ttgtaggtctatcgctttggtcagaaatgaaaccgg

SEQ ID NO: 707 - DNA, 36 bp, Artificial Sequence
  source: /note="Description of Artificial Sequence: Syntheticoligonucleotide"
tgatttcgcgagctaggtggtaaagaacttgacgtt

SEQ ID NO: 708 - DNA, 36 bp, Artificial Sequence
  source: /note="Description of Artificial Sequence: Syntheticoligonucleotide"
cctgtacatcagattgtgacccgcagtatcctaacg

SEQ ID NO: 709 - DNA, 36 bp, Artificial Sequence
  source: /note="Description of Artificial Sequence: Syntheticoligonucleotide"
tagaagcttgagcgaagttggacctgacacgacaat

SEQ ID NO: 710 - DNA, 36 bp, Artificial Sequence
  source: /note="Description of Artificial Sequence: Syntheticoligonucleotide"
agggcacgagttaacgctaatgatgtttctcatcgg

SEQ ID NO: 711 - DNA, 36 bp, Artificial Sequence
  source: /note="Description of Artificial Sequence: Syntheticoligonucleotide"
agttaaagagcggcctcaaacggtgacgaaggatac

SEQ ID NO: 712 - DNA, 36 bp, Artificial Sequence
  source: /note="Description of Artificial Sequence: Syntheticoligonucleotide"
tagtgatatgatacctgcgtacctctcacccatagg

SEQ ID NO: 713 - DNA, 36 bp, Artificial Sequence
  source: /note="Description of Artificial Sequence: Syntheticoligonucleotide"
gtgccaaactaatctacgttatcgcaaccgacttac

SEQ ID NO: 714 - DNA, 36 bp, Artificial Sequence
  source: /note="Description of Artificial Sequence: Syntheticoligonucleotide"
gatgagagacttatgtaataggctcacccgctccaa

SEQ ID NO: 715 - DNA, 36 bp, Artificial Sequence
  source: /note="Description of Artificial Sequence: Syntheticoligonucleotide"
ggcgaccattcaactaacacgttgggcattggaaat

SEQ ID NO: 716 - DNA, 36 bp, Artificial Sequence
  source: /note="Description of Artificial Sequence: Syntheticoligonucleotide"
tccttatttcgctgtattcttctcgtacgtgagaac

SEQ ID NO: 717 - DNA, 36 bp, Artificial Sequence
  source: /note="Description of Artificial Sequence: Syntheticoligonucleotide"
gactgaggattcactactcttgtatatcgggtgtcc

SEQ ID NO: 718 - DNA, 36 bp, Artificial Sequence
  source: /note="Description of Artificial Sequence: Syntheticoligonucleotide"
ttagcaagcaatcacgatgcggaagtgaattcgata

SEQ ID NO: 719 - DNA, 36 bp, Artificial Sequence
  source: /note="Description of Artificial Sequence: Syntheticoligonucleotide"
gttggacatagcctctggtaaacattagtgtagcgg

SEQ ID NO: 720 - DNA, 36 bp, Artificial Sequence
  source: /note="Description of Artificial Sequence: Syntheticoligonucleotide"
tagttgcaaacgttattctaagtcgacctcatctcg

SEQ ID NO: 721 - DNA, 36 bp, Artificial Sequence
  source: /note="Description of Artificial Sequence: Syntheticoligonucleotide"
tgatacgaggccattacccagtaatctaagcttcct

SEQ ID NO: 722 - DNA, 36 bp, Artificial Sequence
  source: /note="Description of Artificial Sequence: Syntheticoligonucleotide"
gatgtagctgcttacgaaacaggtgataattgcagc

SEQ ID NO: 723 - DNA, 36 bp, Artificial Sequence
  source: /note="Description of Artificial Sequence: Syntheticoligonucleotide"
aatagcaccacactattgactaaacgaaagccctca

SEQ ID NO: 724 - DNA, 36 bp, Artificial Sequence
  source: /note="Description of Artificial Sequence: Syntheticoligonucleotide"
cctagatgtcactgagatagcccaacccatcaagaa

SEQ ID NO: 725 - DNA, 36 bp, Artificial Sequence
  source: /note="Description of Artificial Sequence: Syntheticoligonucleotide"
taaggactggcctaccgacatcctgtatcaacattc

SEQ ID NO: 726 - DNA, 36 bp, Artificial Sequence
  source: /note="Description of Artificial Sequence: Syntheticoligonucleotide"
tacgactgcgactccatgcgattgtcctgtttaggt

SEQ ID NO: 727 - DNA, 36 bp, Artificial Sequence
  source: /note="Description of Artificial Sequence: Syntheticoligonucleotide"
tgttaatcgccggtatagacaattcagtaaggaggc

SEQ ID NO: 728 - DNA, 36 bp, Artificial Sequence
  source: /note="Description of Artificial Sequence: Syntheticoligonucleotide"
acgtatttcttaggagcctcatcaacctaggactac

SEQ ID NO: 729 - DNA, 36 bp, Artificial Sequence
  source: /note="Description of Artificial Sequence: Syntheticoligonucleotide"
cctgtccggtcaatttgtaacgcccttcgaattgat

SEQ ID NO: 730 - DNA, 36 bp, Artificial Sequence
  source: /note="Description of Artificial Sequence: Syntheticoligonucleotide"
aaacgcgggccaccaattagggattatctagagtga

SEQ ID NO: 731 - DNA, 36 bp, Artificial Sequence
  source: /note="Description of Artificial Sequence: Syntheticoligonucleotide"
ccaaccacagacgtttaacgaccgcaaccattagac

SEQ ID NO: 732 - DNA, 36 bp, Artificial Sequence
  source: /note="Description of Artificial Sequence: Syntheticoligonucleotide"
tgcgacatagaggccgacagttatacacttaaggcg

SEQ ID NO: 733 - DNA, 36 bp, Artificial Sequence
  source: /note="Description of Artificial Sequence: Syntheticoligonucleotide"
aggtgaaatcagacgtcagccacgagagaatgagat

SEQ ID NO: 734 - DNA, 36 bp, Artificial Sequence
  source: /note="Description of Artificial Sequence: Syntheticoligonucleotide"
tcattaattccgaagggtaactaggcggctcgtaat

SEQ ID NO: 735 - DNA, 36 bp, Artificial Sequence
  source: /note="Description of Artificial Sequence: Syntheticoligonucleotide"
gtcttatatggaagccatcgtgtcgtctggaatcca

SEQ ID NO: 736 - DNA, 36 bp, Artificial Sequence
  source: /note="Description of Artificial Sequence: Syntheticoligonucleotide"
ggctttgacaactagtagcgcgaaagtttgcccaat

SEQ ID NO: 737 - DNA, 36 bp, Artificial Sequence
  source: /note="Description of Artificial Sequence: Syntheticoligonucleotide"
tactgagccgctagaacagaccctcgatcgtttaag

SEQ ID NO: 738 - DNA, 36 bp, Artificial Sequence
  source: /note="Description of Artificial Sequence: Syntheticoligonucleotide"
cccagtgatgcaatttcacaaccataccgggagatt

SEQ ID NO: 739 - DNA, 36 bp, Artificial Sequence
  source: /note="Description of Artificial Sequence: Syntheticoligonucleotide"
aagttgtggcaatacctaggcacggcttagtctagc

SEQ ID NO: 740 - DNA, 36 bp, Artificial Sequence
  source: /note="Description of Artificial Sequence: Syntheticoligonucleotide"
agtcagacatacctcatagccgtgaaggtgtaggac

SEQ ID NO: 741 - DNA, 36 bp, Artificial Sequence
  source: /note="Description of Artificial Sequence: Syntheticoligonucleotide"
acgcccgttatcatcgaacctagagcacgttaatcc

SEQ ID NO: 742 - DNA, 36 bp, Artificial Sequence
  source: /note="Description of Artificial Sequence: Syntheticoligonucleotide"
ttaaggccaaatggcagcgagtgtaaaggacctctg

SEQ ID NO: 743 - DNA, 36 bp, Artificial Sequence
  source: /note="Description of Artificial Sequence: Syntheticoligonucleotide"
tcaacacaacacgctttaatccgcctacgatgccct

SEQ ID NO: 744 - DNA, 36 bp, Artificial Sequence
  source: /note="Description of Artificial Sequence: Syntheticoligonucleotide"
tgcaacggaccgactcacacaattactacagagcta

SEQ ID NO: 745 - DNA, 24 bp, Artificial Sequence
  source: /note="Description of Artificial Sequence: Syntheticoligonucleotide"
actggtaacccagacatgatcggt

SEQ ID NO: 746 - DNA, 24 bp, Artificial Sequence
  source: /note="Description of Artificial Sequence: Syntheticoligonucleotide"
catttggtcattggttcaagacga

SEQ ID NO: 747 - DNA, 24 bp, Artificial Sequence
  source: /note="Description of Artificial Sequence: Syntheticoligonucleotide"
ctaatagctcctgtgccctcgtat

SEQ ID NO: 748 - DNA, 24 bp, Artificial Sequence
  source: /note="Description of Artificial Sequence: Syntheticoligonucleotide"
gtggcaacagaaatcaggtggtga

SEQ ID NO: 749 - DNA, 24 bp, Artificial Sequence
  source: /note="Description of Artificial Sequence: Syntheticoligonucleotide"
tagatgccgctgcaggtatggaaa

SEQ ID NO: 750 - DNA, 24 bp, Artificial Sequence
  source: /note="Description of Artificial Sequence: Syntheticoligonucleotide"
actccctgtgggtgagcttaatgg

SEQ ID NO: 751 - DNA, 24 bp, Artificial Sequence
  source: /note="Description of Artificial Sequence: Syntheticoligonucleotide"
ctggtccgttgtggtccttctaac

SEQ ID NO: 752 - DNA, 24 bp, Artificial Sequence
  source: /note="Description of Artificial Sequence: Syntheticoligonucleotide"
aatccgtcgactagcctgagaatt

SEQ ID NO: 753 - DNA, 24 bp, Artificial Sequence
  source: /note="Description of Artificial Sequence: Syntheticoligonucleotide"
agaggactgctaaaggtttgtagg

SEQ ID NO: 754 - DNA, 24 bp, Artificial Sequence
  source: /note="Description of Artificial Sequence: Syntheticoligonucleotide"
cgtaccattgaatctggagacctt

SEQ ID NO: 755 - DNA, 24 bp, Artificial Sequence
  source: /note="Description of Artificial Sequence: Syntheticoligonucleotide"
attcctcacagagaacagacagtt

SEQ ID NO: 756 - DNA, 24 bp, Artificial Sequence
  source: /note="Description of Artificial Sequence: Syntheticoligonucleotide"
cattaaacgacaatggttacgtga

SEQ ID NO: 757 - DNA, 24 bp, Artificial Sequence
  source: /note="Description of Artificial Sequence: Syntheticoligonucleotide"
tcgggatagcatcatctggcatag

SEQ ID NO: 758 - DNA, 24 bp, Artificial Sequence
  source: /note="Description of Artificial Sequence: Syntheticoligonucleotide"
gaataggtagttgtgacgtaggga

SEQ ID NO: 759 - DNA, 24 bp, Artificial Sequence
  source: /note="Description of Artificial Sequence: Syntheticoligonucleotide"
cccgcttggttagctagatagcac

SEQ ID NO: 760 - DNA, 24 bp, Artificial Sequence
  source: /note="Description of Artificial Sequence: Syntheticoligonucleotide"
cactatggcagtaacccttagtat

SEQ ID NO: 761 - DNA, 24 bp, Artificial Sequence
  source: /note="Description of Artificial Sequence: Syntheticoligonucleotide"
cttggtgtctagaaagaaacacct

SEQ ID NO: 762 - DNA, 24 bp, Artificial Sequence
  source: /note="Description of Artificial Sequence: Syntheticoligonucleotide"
gaaagcaaacgttgctccgtctcg

SEQ ID NO: 763 - DNA, 24 bp, Artificial Sequence
  source: /note="Description of Artificial Sequence: Syntheticoligonucleotide"
aaacactttatgtaagattccgtc

SEQ ID NO: 764 - DNA, 24 bp, Artificial Sequence
  source: /note="Description of Artificial Sequence: Syntheticoligonucleotide"
tacccgacttaaatcacggtactg

SEQ ID NO: 765 - DNA, 24 bp, Artificial Sequence
  source: /note="Description of Artificial Sequence: Syntheticoligonucleotide"
ccaaatccggtataacgtagagac

SEQ ID NO: 766 - DNA, 24 bp, Artificial Sequence
  source: /note="Description of Artificial Sequence: Syntheticoligonucleotide"
gctttcccgcagtctcaaagaaag

SEQ ID NO: 767 - DNA, 24 bp, Artificial Sequence
  source: /note="Description of Artificial Sequence: Syntheticoligonucleotide"
cactgagccaagccgtgtaagtac

SEQ ID NO: 768 - DNA, 24 bp, Artificial Sequence
  source: /note="Description of Artificial Sequence: Syntheticoligonucleotide"
gttatttacctacagaacaccgag

SEQ ID NO: 769 - DNA, 24 bp, Artificial Sequence
  source: /note="Description of Artificial Sequence: Syntheticoligonucleotide"
ggccaggttcgcttgggatgttac

SEQ ID NO: 770 - DNA, 24 bp, Artificial Sequence
  source: /note="Description of Artificial Sequence: Syntheticoligonucleotide"
ttcgcgactgttgcagcactaccc

SEQ ID NO: 771 - DNA, 24 bp, Artificial Sequence
  source: /note="Description of Artificial Sequence: Syntheticoligonucleotide"
tatgtgtgtgataaatgactgact

SEQ ID NO: 772 - DNA, 24 bp, Artificial Sequence
  source: /note="Description of Artificial Sequence: Syntheticoligonucleotide"
cgaagggtgatgagcatctttgaa

SEQ ID NO: 773 - DNA, 24 bp, Artificial Sequence
  source: /note="Description of Artificial Sequence: Syntheticoligonucleotide"
agggacaggcgcactcgagactat

SEQ ID NO: 774 - DNA, 24 bp, Artificial Sequence
  source: /note="Description of Artificial Sequence: Syntheticoligonucleotide"
gtaatacaggtagatcgcgtagga

SEQ ID NO: 775 - DNA, 24 bp, Artificial Sequence
  source: /note="Description of Artificial Sequence: Syntheticoligonucleotide"
tgtgacggatcagcttccgcagaa

SEQ ID NO: 776 - DNA, 24 bp, Artificial Sequence
  source: /note="Description of Artificial Sequence: Syntheticoligonucleotide"
ggttgtattcaagcggcggaacca

SEQ ID NO: 777 - DNA, 24 bp, Artificial Sequence
  source: /note="Description of Artificial Sequence: Syntheticoligonucleotide"
agcgtactgctcgtaaacgctcac

SEQ ID NO: 778 - DNA, 24 bp, Artificial Sequence
  source: /note="Description of Artificial Sequence: Syntheticoligonucleotide"
tggcgttccaacaataacaccctg

SEQ ID NO: 779 - DNA, 24 bp, Artificial Sequence
  source: /note="Description of Artificial Sequence: Syntheticoligonucleotide"
cgatgttgaaagttcatttgagcg

SEQ ID NO: 780 - DNA, 24 bp, Artificial Sequence
  source: /note="Description of Artificial Sequence: Syntheticoligonucleotide"
ctaagaagcatctgcccaatacca

SEQ ID NO: 781 - DNA, 24 bp, Artificial Sequence
  source: /note="Description of Artificial Sequence: Syntheticoligonucleotide"
cgtaaatttcgtgcatggcaagat

SEQ ID NO: 782 - DNA, 24 bp, Artificial Sequence
  source: /note="Description of Artificial Sequence: Syntheticoligonucleotide"
gcgggttcctgtttaagtagtata

SEQ ID NO: 783 - DNA, 24 bp, Artificial Sequence
  source: /note="Description of Artificial Sequence: Syntheticoligonucleotide"
gcatttctgaatgtagggatctac

SEQ ID NO: 784 - DNA, 24 bp, Artificial Sequence
  source: /note="Description of Artificial Sequence: Syntheticoligonucleotide"
attatattcgcgcacttagcacac

SEQ ID NO: 785 - DNA, 24 bp, Artificial Sequence
  source: /note="Description of Artificial Sequence: Syntheticoligonucleotide"
taactaactcattatctgcttgcg

SEQ ID NO: 786 - DNA, 24 bp, Artificial Sequence
  source: /note="Description of Artificial Sequence: Syntheticoligonucleotide"
ttacaagcctaggagcgtgatact

SEQ ID NO: 787 - DNA, 24 bp, Artificial Sequence
  source: /note="Description of Artificial Sequence: Syntheticoligonucleotide"
cacatagattgacgaattgtatgg

SEQ ID NO: 788 - DNA, 24 bp, Artificial Sequence
  source: /note="Description of Artificial Sequence: Syntheticoligonucleotide"
cttatgttgagcaacccgtcatgt

SEQ ID NO: 789 - DNA, 24 bp, Artificial Sequence
  source: /note="Description of Artificial Sequence: Syntheticoligonucleotide"
tgtcgttacgtttattaaccgccg

SEQ ID NO: 790 - DNA, 24 bp, Artificial Sequence
  source: /note="Description of Artificial Sequence: Syntheticoligonucleotide"
caggaatgtcggaaatacttgaaa

SEQ ID NO: 791 - DNA, 24 bp, Artificial Sequence
  source: /note="Description of Artificial Sequence: Syntheticoligonucleotide"
gacgtacgctagtctagttacgaa

SEQ ID NO: 792 - DNA, 24 bp, Artificial Sequence
  source: /note="Description of Artificial Sequence: Syntheticoligonucleotide"
ttatacgcggcaccgaattccact

SEQ ID NO: 793 - DNA, 24 bp, Artificial Sequence
  source: /note="Description of Artificial Sequence: Syntheticoligonucleotide"
tgtcttgtgtaaggatgaggcaaa

SEQ ID NO: 794 - DNA, 24 bp, Artificial Sequence
  source: /note="Description of Artificial Sequence: Syntheticoligonucleotide"
aaggcagggtgggttgttgatatc

SEQ ID NO: 795 - DNA, 24 bp, Artificial Sequence
  source: /note="Description of Artificial Sequence: Syntheticoligonucleotide"
cgcagctaatttaactatatctgg

SEQ ID NO: 796 - DNA, 24 bp, Artificial Sequence
  source: /note="Description of Artificial Sequence: Syntheticoligonucleotide"
aggtcgcgggacgggtaatattca

SEQ ID NO: 797 - DNA, 24 bp, Artificial Sequence
  source: /note="Description of Artificial Sequence: Syntheticoligonucleotide"
ggaataacatgtcccaattgatga

SEQ ID NO: 798 - DNA, 24 bp, Artificial Sequence
  source: /note="Description of Artificial Sequence: Syntheticoligonucleotide"
acggcccttgactttgttctataa

SEQ ID NO: 799 - DNA, 24 bp, Artificial Sequence
  source: /note="Description of Artificial Sequence: Syntheticoligonucleotide"
tgttaacttagggcctactgaggc

SEQ ID NO: 800 - DNA, 24 bp, Artificial Sequence
  source: /note="Description of Artificial Sequence: Syntheticoligonucleotide"
gaattaccactccattggcctgct

SEQ ID NO: 801 - DNA, 24 bp, Artificial Sequence
  source: /note="Description of Artificial Sequence: Syntheticoligonucleotide"
gctttagatatgagccgacactta

SEQ ID NO: 802 - DNA, 24 bp, Artificial Sequence
  source: /note="Description of Artificial Sequence: Syntheticoligonucleotide"
ggctcgaggatcatcatatcaatg

SEQ ID NO: 803 - DNA, 24 bp, Artificial Sequence
  source: /note="Description of Artificial Sequence: Syntheticoligonucleotide"
gcgtcgaaagacgccagtacttcc

SEQ ID NO: 804 - DNA, 24 bp, Artificial Sequence
  source: /note="Description of Artificial Sequence: Syntheticoligonucleotide"
tatcttatgggtttctcgtatatt

SEQ ID NO: 805 - DNA, 24 bp, Artificial Sequence
  source: /note="Description of Artificial Sequence: Syntheticoligonucleotide"
attaagacccaggtagtagctgtt

SEQ ID NO: 806 - DNA, 24 bp, Artificial Sequence
  source: /note="Description of Artificial Sequence: Syntheticoligonucleotide"
ccggcctcatgttagatcagtggg

SEQ ID NO: 807 - DNA, 24 bp, Artificial Sequence
  source: /note="Description of Artificial Sequence: Syntheticoligonucleotide"
gggcagcggttagtacgaggtcgg

SEQ ID NO: 808 - DNA, 24 bp, Artificial Sequence
  source: /note="Description of Artificial Sequence: Syntheticoligonucleotide"
aggcccggatctttgtcacgaacg

SEQ ID NO: 809 - DNA, 24 bp, Artificial Sequence
  source: /note="Description of Artificial Sequence: Syntheticoligonucleotide"
ttgatacgtactctgaacgacgac

SEQ ID NO: 810 - DNA, 24 bp, Artificial Sequence
  source: /note="Description of Artificial Sequence: Syntheticoligonucleotide"
agaggatctggaggccgtgctgtt

SEQ ID NO: 811 - DNA, 24 bp, Artificial Sequence
  source: /note="Description of Artificial Sequence: Syntheticoligonucleotide"
cggaaagactcagcgtttcagtaa

SEQ ID NO: 812 - DNA, 24 bp, Artificial Sequence
  source: /note="Description of Artificial Sequence: Syntheticoligonucleotide"
aattgaatggctcgttcgaagaat

SEQ ID NO: 813 - DNA, 24 bp, Artificial Sequence
  source: /note="Description of Artificial Sequence: Syntheticoligonucleotide"
acaaatagtgcacgcgctctctaa

SEQ ID NO: 814 - DNA, 24 bp, Artificial Sequence
  source: /note="Description of Artificial Sequence: Syntheticoligonucleotide"
agctatttaatctcaagaccagat

SEQ ID NO: 815 - DNA, 24 bp, Artificial Sequence
  source: /note="Description of Artificial Sequence: Syntheticoligonucleotide"
actttgtgcgaacagattaagcta

SEQ ID NO: 816 - DNA, 24 bp, Artificial Sequence
  source: /note="Description of Artificial Sequence: Syntheticoligonucleotide"
ttagtcgtaattgagaaattgcgg

SEQ ID NO: 817 - DNA, 24 bp, Artificial Sequence
  source: /note="Description of Artificial Sequence: Syntheticoligonucleotide"
tgagagctgaaatatagtgcggtc

SEQ ID NO: 818 - DNA, 24 bp, Artificial Sequence
  source: /note="Description of Artificial Sequence: Syntheticoligonucleotide"
ggaatagagtctaaacaagccgct

SEQ ID NO: 819 - DNA, 24 bp, Artificial Sequence
  source: /note="Description of Artificial Sequence: Syntheticoligonucleotide"
agtgattggactgtcgttggtatg

SEQ ID NO: 820 - DNA, 24 bp, Artificial Sequence
  source: /note="Description of Artificial Sequence: Syntheticoligonucleotide"
agtcgttgcggtgatttcgatata

SEQ ID NO: 821 - DNA, 24 bp, Artificial Sequence
  source: /note="Description of Artificial Sequence: Syntheticoligonucleotide"
gccgtccgggatttctttaaacat

SEQ ID NO: 822 - DNA, 24 bp, Artificial Sequence
  source: /note="Description of Artificial Sequence: Syntheticoligonucleotide"
gatcaacacatgatagtatagctg

SEQ ID NO: 823 - DNA, 24 bp, Artificial Sequence
  source: /note="Description of Artificial Sequence: Syntheticoligonucleotide"
cggatgatataccacgctgttatt

SEQ ID NO: 824 - DNA, 24 bp, Artificial Sequence
  source: /note="Description of Artificial Sequence: Syntheticoligonucleotide"
cgctgcagttgaccttctacatac

SEQ ID NO: 825 - DNA, 24 bp, Artificial Sequence
  source: /note="Description of Artificial Sequence: Syntheticoligonucleotide"
ggacctatacaagtcaaatagcga

SEQ ID NO: 826 - DNA, 24 bp, Artificial Sequence
  source: /note="Description of Artificial Sequence: Syntheticoligonucleotide"
aagaatcgggtaccaacccacttt

SEQ ID NO: 827 - DNA, 24 bp, Artificial Sequence
  source: /note="Description of Artificial Sequence: Syntheticoligonucleotide"
atggttctagacaactaatgcgtc

SEQ ID NO: 828 - DNA, 24 bp, Artificial Sequence
  source: /note="Description of Artificial Sequence: Syntheticoligonucleotide"
attgaccctgatggtgttaatagt

SEQ ID NO: 829 - DNA, 24 bp, Artificial Sequence
  source: /note="Description of Artificial Sequence: Syntheticoligonucleotide"
gcgttcacatggacctaactcgta

SEQ ID NO: 830 - DNA, 24 bp, Artificial Sequence
  source: /note="Description of Artificial Sequence: Syntheticoligonucleotide"
gcaacaggtgaaagaccttgacta

SEQ ID NO: 831 - DNA, 24 bp, Artificial Sequence
  source: /note="Description of Artificial Sequence: Syntheticoligonucleotide"
aatactcgggcgtgctaataatat

SEQ ID NO: 832 - DNA, 24 bp, Artificial Sequence
  source: /note="Description of Artificial Sequence: Syntheticoligonucleotide"
cgacacaacgccacgtagattcat

SEQ ID NO: 833 - DNA, 24 bp, Artificial Sequence
  source: /note="Description of Artificial Sequence: Syntheticoligonucleotide"
tttattcgtccacggtgagtaact

SEQ ID NO: 834 - DNA, 24 bp, Artificial Sequence
  source: /note="Description of Artificial Sequence: Syntheticoligonucleotide"
ctgtagcttcaagtttcgcgaggg

SEQ ID NO: 835 - DNA, 24 bp, Artificial Sequence
  source: /note="Description of Artificial Sequence: Syntheticoligonucleotide"
agacagtaagtagaggaatcagtc

SEQ ID NO: 836 - DNA, 24 bp, Artificial Sequence
  source: /note="Description of Artificial Sequence: Syntheticoligonucleotide"
attggtgtcatcctgcctgattgt

SEQ ID NO: 837 - DNA, 24 bp, Artificial Sequence
  source: /note="Description of Artificial Sequence: Syntheticoligonucleotide"
caaggctccaaggcttacgctcca

SEQ ID NO: 838 - DNA, 24 bp, Artificial Sequence
  source: /note="Description of Artificial Sequence: Syntheticoligonucleotide"
aagttctaaggttaagacgcttcg

SEQ ID NO: 839 - DNA, 24 bp, Artificial Sequence
  source: /note="Description of Artificial Sequence: Syntheticoligonucleotide"
acgaaccagaaccctgaatgttcg

SEQ ID NO: 840 - DNA, 24 bp, Artificial Sequence
  source: /note="Description of Artificial Sequence: Syntheticoligonucleotide"
cctcggatgtagagcgattgagga

SEQ ID NO: 841 - DNA, 24 bp, Artificial Sequence
  source: /note="Description of Artificial Sequence: Syntheticoligonucleotide"
tgtcctcattaccgtcccaacaca

SEQ ID NO: 842 - DNA, 24 bp, Artificial Sequence
  source: /note="Description of Artificial Sequence: Syntheticoligonucleotide"
agtgctaacttgtttgagtaagct

SEQ ID NO: 843 - DNA, 24 bp, Artificial Sequence
  source: /note="Description of Artificial Sequence: Syntheticoligonucleotide"
cacaaggtcggtgctgcaattcga

SEQ ID NO: 844 - DNA, 24 bp, Artificial Sequence
  source: /note="Description of Artificial Sequence: Syntheticoligonucleotide"
atcaccattctctctcacttcgct

SEQ ID NO: 845 - DNA, 24 bp, Artificial Sequence
  source: /note="Description of Artificial Sequence: Syntheticoligonucleotide"
catacgattaacccttccggtcca

SEQ ID NO: 846 - DNA, 24 bp, Artificial Sequence
  source: /note="Description of Artificial Sequence: Syntheticoligonucleotide"
cgagattgagttagagtccctatt

SEQ ID NO: 847 - DNA, 24 bp, Artificial Sequence
  source: /note="Description of Artificial Sequence: Syntheticoligonucleotide"
aaggtttgaccatttgacgagcct

SEQ ID NO: 848 - DNA, 24 bp, Artificial Sequence
  source: /note="Description of Artificial Sequence: Syntheticoligonucleotide"
taagttcgtcgagtagaacggtcc

SEQ ID NO: 849 - DNA, 24 bp, Artificial Sequence
  source: /note="Description of Artificial Sequence: Syntheticoligonucleotide"
agaggacatgtagcaactgtcgca

SEQ ID NO: 850 - DNA, 24 bp, Artificial Sequence
  source: /note="Description of Artificial Sequence: Syntheticoligonucleotide"
agcatccaaagcacgattcacggc

SEQ ID NO: 851 - DNA, 24 bp, Artificial Sequence
  source: /note="Description of Artificial Sequence: Syntheticoligonucleotide"
ctcgtttctctgagtagcagatag

SEQ ID NO: 852 - DNA, 24 bp, Artificial Sequence
  source: /note="Description of Artificial Sequence: Syntheticoligonucleotide"
tgccacatcaataatcccaggatc

SEQ ID NO: 853 - DNA, 24 bp, Artificial Sequence
  source: /note="Description of Artificial Sequence: Syntheticoligonucleotide"
aacctaagaaagctgtggttccgc

SEQ ID NO: 854 - DNA, 24 bp, Artificial Sequence
  source: /note="Description of Artificial Sequence: Syntheticoligonucleotide"
atgacgtgcatctaccgcacagat

SEQ ID NO: 855 - DNA, 24 bp, Artificial Sequence
  source: /note="Description of Artificial Sequence: Syntheticoligonucleotide"
gtttcccgaatcgaataacgtcgg

SEQ ID NO: 856 - DNA, 24 bp, Artificial Sequence
  source: /note="Description of Artificial Sequence: Syntheticoligonucleotide"
gttacgttactacccagcgccaga

SEQ ID NO: 857 - DNA, 24 bp, Artificial Sequence
  source: /note="Description of Artificial Sequence: Syntheticoligonucleotide"
aactgcggttgttctgcgcccact

SEQ ID NO: 858 - DNA, 24 bp, Artificial Sequence
  source: /note="Description of Artificial Sequence: Syntheticoligonucleotide"
ctcatgtcggtgtccctacgtacg

SEQ ID NO: 859 - DNA, 24 bp, Artificial Sequence
  source: /note="Description of Artificial Sequence: Syntheticoligonucleotide"
tgttgttacgaagacggcgaaatg

SEQ ID NO: 860 - DNA, 24 bp, Artificial Sequence
  source: /note="Description of Artificial Sequence: Syntheticoligonucleotide"
aaacccagtcactccgtcactcgt

SEQ ID NO: 861 - DNA, 24 bp, Artificial Sequence
  source: /note="Description of Artificial Sequence: Syntheticoligonucleotide"
gctacacgcaccgcgctgaagtac

SEQ ID NO: 862 - DNA, 24 bp, Artificial Sequence
  source: /note="Description of Artificial Sequence: Syntheticoligonucleotide"
agttcgttacgtgttcaagagcca

SEQ ID NO: 863 - DNA, 24 bp, Artificial Sequence
  source: /note="Description of Artificial Sequence: Syntheticoligonucleotide"
ccgttctgggatcttattgtttaa

SEQ ID NO: 864 - DNA, 24 bp, Artificial Sequence
  source: /note="Description of Artificial Sequence: Syntheticoligonucleotide"
tagtgcaagatggagagtgttcat

SEQ ID NO: 865 - DNA, 24 bp, Artificial Sequence
  source: /note="Description of Artificial Sequence: Syntheticoligonucleotide"
aagtggaatcacagaatctgaggg

SEQ ID NO: 866 - DNA, 24 bp, Artificial Sequence
  source: /note="Description of Artificial Sequence: Syntheticoligonucleotide"
cctgctgttcacgtatatgatgtc

SEQ ID NO: 867 - DNA, 24 bp, Artificial Sequence
  source: /note="Description of Artificial Sequence: Syntheticoligonucleotide"
ttcacaaaccctctccacagtagc

SEQ ID NO: 868 - DNA, 24 bp, Artificial Sequence
  source: /note="Description of Artificial Sequence: Syntheticoligonucleotide"
tttattgctccctagcattgctgt

SEQ ID NO: 869 - DNA, 24 bp, Artificial Sequence
  source: /note="Description of Artificial Sequence: Syntheticoligonucleotide"
atccaaatggaaagagaacgtctc

SEQ ID NO: 870 - DNA, 24 bp, Artificial Sequence
  source: /note="Description of Artificial Sequence: Syntheticoligonucleotide"
gagtatgggccacaacttctggtt

SEQ ID NO: 871 - DNA, 24 bp, Artificial Sequence
  source: /note="Description of Artificial Sequence: Syntheticoligonucleotide"
ctctgtggtggagttacgattcgc

SEQ ID NO: 872 - DNA, 24 bp, Artificial Sequence
  source: /note="Description of Artificial Sequence: Syntheticoligonucleotide"
gagcgaatataagggctacgaccg

SEQ ID NO: 873 - DNA, 24 bp, Artificial Sequence
  source: /note="Description of Artificial Sequence: Syntheticoligonucleotide"
atctaaacggtcgcaagcggaacc

SEQ ID NO: 874 - DNA, 24 bp, Artificial Sequence
  source: /note="Description of Artificial Sequence: Syntheticoligonucleotide"
ttacgacgtatcacgcgaacagcc

SEQ ID NO: 875 - DNA, 24 bp, Artificial Sequence
  source: /note="Description of Artificial Sequence: Syntheticoligonucleotide"
acgtgacaaatactagacatggtt

SEQ ID NO: 876 - DNA, 24 bp, Artificial Sequence
  source: /note="Description of Artificial Sequence: Syntheticoligonucleotide"
caaggacaggtgtggcgaagtaca

SEQ ID NO: 877 - DNA, 24 bp, Artificial Sequence
  source: /note="Description of Artificial Sequence: Syntheticoligonucleotide"
ctaccggctctgaaggaccctagt

SEQ ID NO: 878 - DNA, 24 bp, Artificial Sequence
  source: /note="Description of Artificial Sequence: Syntheticoligonucleotide"
acaccgtccctataccagtgtaaa

SEQ ID NO: 879 - DNA, 24 bp, Artificial Sequence
  source: /note="Description of Artificial Sequence: Syntheticoligonucleotide"
tcctccaaatcatatttaacccac

SEQ ID NO: 880 - DNA, 24 bp, Artificial Sequence
  source: /note="Description of Artificial Sequence: Syntheticoligonucleotide"
actgcgaatagccgtcagaacttt

SEQ ID NO: 881 - DNA, 24 bp, Artificial Sequence
  source: /note="Description of Artificial Sequence: Syntheticoligonucleotide"
ctattctgcacacgaaggcaatct

SEQ ID NO: 882 - DNA, 24 bp, Artificial Sequence
  source: /note="Description of Artificial Sequence: Syntheticoligonucleotide"
gacgagcccgtatcgccttggaat

SEQ ID NO: 883 - DNA, 24 bp, Artificial Sequence
  source: /note="Description of Artificial Sequence: Syntheticoligonucleotide"
aacgtctgtcaggcgaggcttata

SEQ ID NO: 884 - DNA, 24 bp, Artificial Sequence
  source: /note="Description of Artificial Sequence: Syntheticoligonucleotide"
gtgtagatgggcgttaagccagca

SEQ ID NO: 885 - DNA, 24 bp, Artificial Sequence
  source: /note="Description of Artificial Sequence: Syntheticoligonucleotide"
ctatgtgcggtttataacctcccg

SEQ ID NO: 886 - DNA, 24 bp, Artificial Sequence
  source: /note="Description of Artificial Sequence: Syntheticoligonucleotide"
gctgctgctcacaggcgaactgat

SEQ ID NO: 887 - DNA, 24 bp, Artificial Sequence
  source: /note="Description of Artificial Sequence: Syntheticoligonucleotide"
caggtatacgtagttgataccttt

SEQ ID NO: 888 - DNA, 24 bp, Artificial Sequence
  source: /note="Description of Artificial Sequence: Syntheticoligonucleotide"
aactcatagaaactatccctggag

SEQ ID NO: 889 - DNA, 24 bp, Artificial Sequence
  source: /note="Description of Artificial Sequence: Syntheticoligonucleotide"
ttacagacagttcgtcaaatagag

SEQ ID NO: 890 - DNA, 24 bp, Artificial Sequence
  source: /note="Description of Artificial Sequence: Syntheticoligonucleotide"
gagacactttccttcatataactc

SEQ ID NO: 891 - DNA, 24 bp, Artificial Sequence
  source: /note="Description of Artificial Sequence: Syntheticoligonucleotide"
atatttgtgggtacacgaccagac

SEQ ID NO: 892 - DNA, 24 bp, Artificial Sequence
  source: /note="Description of Artificial Sequence: Syntheticoligonucleotide"
gactggtttcgctatctggatgtt

SEQ ID NO: 893 - DNA, 24 bp, Artificial Sequence
  source: /note="Description of Artificial Sequence: Syntheticoligonucleotide"
aaatggtggatcgagcgctttagt

SEQ ID NO: 894 - DNA, 24 bp, Artificial Sequence
  source: /note="Description of Artificial Sequence: Syntheticoligonucleotide"
cgcccagtgttagactacatgtcc

SEQ ID NO: 895 - DNA, 24 bp, Artificial Sequence
  source: /note="Description of Artificial Sequence: Syntheticoligonucleotide"
ccaggttgaagcgagttcgaagat

SEQ ID NO: 896 - DNA, 24 bp, Artificial Sequence
  source: /note="Description of Artificial Sequence: Syntheticoligonucleotide"
tagtaatcgcaattgagcacggga

SEQ ID NO: 897 - DNA, 24 bp, Artificial Sequence
  source: /note="Description of Artificial Sequence: Syntheticoligonucleotide"
tggcaaactccggcgagaaattga

SEQ ID NO: 898 - DNA, 24 bp, Artificial Sequence
  source: /note="Description of Artificial Sequence: Syntheticoligonucleotide"
tccatgcgtccatagtatagtgat

SEQ ID NO: 899 - DNA, 24 bp, Artificial Sequence
  source: /note="Description of Artificial Sequence: Syntheticoligonucleotide"
gctattgcatctaatcaaaccgtg

SEQ ID NO: 900 - DNA, 24 bp, Artificial Sequence
  source: /note="Description of Artificial Sequence: Syntheticoligonucleotide"
tcggataatgtattcagagagtag

SEQ ID NO: 901 - DNA, 24 bp, Artificial Sequence
  source: /note="Description of Artificial Sequence: Syntheticoligonucleotide"
gttgcacaatcaacttaccagcgg

SEQ ID NO: 902 - DNA, 24 bp, Artificial Sequence
  source: /note="Description of Artificial Sequence: Syntheticoligonucleotide"
ctcttcttatgtcgctttattcct

SEQ ID NO: 903 - DNA, 24 bp, Artificial Sequence
  source: /note="Description of Artificial Sequence: Syntheticoligonucleotide"
atgttctcatcacttaggagtcag

SEQ ID NO: 904 - DNA, 24 bp, Artificial Sequence
  source: /note="Description of Artificial Sequence: Syntheticoligonucleotide"
aaggcgtagcactaacgaacgatt

SEQ ID NO: 905 - DNA, 24 bp, Artificial Sequence
  source: /note="Description of Artificial Sequence: Syntheticoligonucleotide"
acaaatggtctccgatacaggttg

SEQ ID NO: 906 - DNA, 24 bp, Artificial Sequence
  source: /note="Description of Artificial Sequence: Syntheticoligonucleotide"
ctgaatcttattgcaaacgttgat

SEQ ID NO: 907 - DNA, 24 bp, Artificial Sequence
  source: /note="Description of Artificial Sequence: Syntheticoligonucleotide"
aatgacccattaccggagcatagt

SEQ ID NO: 908 - DNA, 24 bp, Artificial Sequence
  source: /note="Description of Artificial Sequence: Syntheticoligonucleotide"
tggacaaagcattcgtcgatgtag

SEQ ID NO: 909 - DNA, 24 bp, Artificial Sequence
  source: /note="Description of Artificial Sequence: Syntheticoligonucleotide"
aaatcagttatcacaccacgataa

SEQ ID NO: 910 - DNA, 24 bp, Artificial Sequence
  source: /note="Description of Artificial Sequence: Syntheticoligonucleotide"
acccgatagagtcactgtagatcc

SEQ ID NO: 911 - DNA, 24 bp, Artificial Sequence
  source: /note="Description of Artificial Sequence: Syntheticoligonucleotide"
tcctacagccatccggtcaggaat

SEQ ID NO: 912 - DNA, 24 bp, Artificial Sequence
  source: /note="Description of Artificial Sequence: Syntheticoligonucleotide"
gttagcgtacctcagcgtcagcat

SEQ ID NO: 913 - DNA, 24 bp, Artificial Sequence
  source: /note="Description of Artificial Sequence: Syntheticoligonucleotide"
ttaacagatatggccgctaattgt

SEQ ID NO: 914 - DNA, 24 bp, Artificial Sequence
  source: /note="Description of Artificial Sequence: Syntheticoligonucleotide"
actactccgaggattctttatgca

SEQ ID NO: 915 - DNA, 24 bp, Artificial Sequence
  source: /note="Description of Artificial Sequence: Syntheticoligonucleotide"
ccgcaatgtttaactggcctgtcc

SEQ ID NO: 916 - DNA, 24 bp, Artificial Sequence
  source: /note="Description of Artificial Sequence: Syntheticoligonucleotide"
tagggattaaccaccgggcgcaaa

SEQ ID NO: 917 - DNA, 24 bp, Artificial Sequence
  source: /note="Description of Artificial Sequence: Syntheticoligonucleotide"
gccagcaatttgcagacaccaacc

SEQ ID NO: 918 - DNA, 24 bp, Artificial Sequence
  source: /note="Description of Artificial Sequence: Syntheticoligonucleotide"
tattgacagccggagatacagcgt

SEQ ID NO: 919 - DNA, 24 bp, Artificial Sequence
  source: /note="Description of Artificial Sequence: Syntheticoligonucleotide"
gcaccgactgcagactaaagtgga

SEQ ID NO: 920 - DNA, 24 bp, Artificial Sequence
  source: /note="Description of Artificial Sequence: Syntheticoligonucleotide"
gatcaatgggaagccttaattact

SEQ ID NO: 921 - DNA, 24 bp, Artificial Sequence
  source: /note="Description of Artificial Sequence: Syntheticoligonucleotide"
ctgtgctaccgaaggtatattctg

SEQ ID NO: 922 - DNA, 24 bp, Artificial Sequence
  source: /note="Description of Artificial Sequence: Syntheticoligonucleotide"
aagcgcgatgatcaacagtttcgg

SEQ ID NO: 923 - DNA, 24 bp, Artificial Sequence
  source: /note="Description of Artificial Sequence: Syntheticoligonucleotide"
tcccagacaagatcgccgagtcat

SEQ ID NO: 924 - DNA, 24 bp, Artificial Sequence
  source: /note="Description of Artificial Sequence: Syntheticoligonucleotide"
accaacactttaacgtagtgaccc

SEQ ID NO: 925 - DNA, 24 bp, Artificial Sequence
  source: /note="Description of Artificial Sequence: Syntheticoligonucleotide"
gcacggatccataacggtgttgaa

SEQ ID NO: 926 - DNA, 24 bp, Artificial Sequence
  source: /note="Description of Artificial Sequence: Syntheticoligonucleotide"
gtgccgatactccatacagactga

SEQ ID NO: 927 - DNA, 24 bp, Artificial Sequence
  source: /note="Description of Artificial Sequence: Syntheticoligonucleotide"
agatccaagctactattgcccgca

SEQ ID NO: 928 - DNA, 24 bp, Artificial Sequence
  source: /note="Description of Artificial Sequence: Syntheticoligonucleotide"
tgtgagcgacggtaaaccggaatt

SEQ ID NO: 929 - DNA, 24 bp, Artificial Sequence
  source: /note="Description of Artificial Sequence: Syntheticoligonucleotide"
cgcctaatttcgcacaacacaact

SEQ ID NO: 930 - DNA, 24 bp, Artificial Sequence
  source: /note="Description of Artificial Sequence: Syntheticoligonucleotide"
ttaacacactcagccaggcaacgt

SEQ ID NO: 931 - DNA, 24 bp, Artificial Sequence
  source: /note="Description of Artificial Sequence: Syntheticoligonucleotide"
tgggttaccagttagtcgtgtctc

SEQ ID NO: 932 - DNA, 24 bp, Artificial Sequence
  source: /note="Description of Artificial Sequence: Syntheticoligonucleotide"
aatgaccaaatgcaagccctccat

SEQ ID NO: 933 - DNA, 24 bp, Artificial Sequence
  source: /note="Description of Artificial Sequence: Syntheticoligonucleotide"
aggagctattagtgtagcgaaagg

SEQ ID NO: 934 - DNA, 24 bp, Artificial Sequence
  source: /note="Description of Artificial Sequence: Syntheticoligonucleotide"
ttctgttgccaccgcatcagttta

SEQ ID NO: 935 - DNA, 24 bp, Artificial Sequence
  source: /note="Description of Artificial Sequence: Syntheticoligonucleotide"
cagcggcatctattcatgacatgt

SEQ ID NO: 936 - DNA, 24 bp, Artificial Sequence
  source: /note="Description of Artificial Sequence: Syntheticoligonucleotide"
cccacagggagttggagtctaaac

SEQ ID NO: 937 - DNA, 24 bp, Artificial Sequence
  source: /note="Description of Artificial Sequence: Syntheticoligonucleotide"
acaacggaccagagagtgcatata

SEQ ID NO: 938 - DNA, 24 bp, Artificial Sequence
  source: /note="Description of Artificial Sequence: Syntheticoligonucleotide"
agtcgacggatttaccgtcactcg

SEQ ID NO: 939 - DNA, 24 bp, Artificial Sequence
  source: /note="Description of Artificial Sequence: Syntheticoligonucleotide"
tagcagtcctctgttggtctctgc

SEQ ID NO: 940 - DNA, 24 bp, Artificial Sequence
  source: /note="Description of Artificial Sequence: Syntheticoligonucleotide"
ttcaatggtacgaccatccgactc

SEQ ID NO: 941 - DNA, 24 bp, Artificial Sequence
  source: /note="Description of Artificial Sequence: Syntheticoligonucleotide"
tctgtgaggaattctccttccgag

SEQ ID NO: 942 - DNA, 24 bp, Artificial Sequence
  source: /note="Description of Artificial Sequence: Syntheticoligonucleotide"
tgtcgtttaatgtctcccagcgtt

SEQ ID NO: 943 - DNA, 24 bp, Artificial Sequence
  source: /note="Description of Artificial Sequence: Syntheticoligonucleotide"
atgctatcccgacgatgaacggtt

SEQ ID NO: 944 - DNA, 24 bp, Artificial Sequence
  source: /note="Description of Artificial Sequence: Syntheticoligonucleotide"
aactacctattcgagtgtggtctt

SEQ ID NO: 945 - DNA, 24 bp, Artificial Sequence
  source: /note="Description of Artificial Sequence: Syntheticoligonucleotide"
taaccaagcgggctctttataggc

SEQ ID NO: 946 - DNA, 24 bp, Artificial Sequence
  source: /note="Description of Artificial Sequence: Syntheticoligonucleotide"
actgccatagtgccgacgcgtaga

SEQ ID NO: 947 - DNA, 24 bp, Artificial Sequence
  source: /note="Description of Artificial Sequence: Syntheticoligonucleotide"
ctagacaccaagtatccacaggca

SEQ ID NO: 948 - DNA, 24 bp, Artificial Sequence
  source: /note="Description of Artificial Sequence: Syntheticoligonucleotide"
acgtttgctttcagttaatcggag

SEQ ID NO: 949 - DNA, 24 bp, Artificial Sequence
  source: /note="Description of Artificial Sequence: Syntheticoligonucleotide"
cataaagtgtttggagatggtagg

SEQ ID NO: 950 - DNA, 24 bp, Artificial Sequence
  source: /note="Description of Artificial Sequence: Syntheticoligonucleotide"
ttaagtcgggtagacgatgcggat

SEQ ID NO: 951 - DNA, 24 bp, Artificial Sequence
  source: /note="Description of Artificial Sequence: Syntheticoligonucleotide"
taccggatttgggtattctctgag

SEQ ID NO: 952 - DNA, 24 bp, Artificial Sequence
  source: /note="Description of Artificial Sequence: Syntheticoligonucleotide"
ctgcgggaaagcggttcggtaact

SEQ ID NO: 953 - DNA, 24 bp, Artificial Sequence
  source: /note="Description of Artificial Sequence: Syntheticoligonucleotide"
cttggctcagtgcccgtttcatat

SEQ ID NO: 954 - DNA, 24 bp, Artificial Sequence
  source: /note="Description of Artificial Sequence: Syntheticoligonucleotide"
taggtaaataacgagtaatcgcac

SEQ ID NO: 955 - DNA, 24 bp, Artificial Sequence
  source: /note="Description of Artificial Sequence: Syntheticoligonucleotide"
gcgaacctggcctttagtacccaa

SEQ ID NO: 956 - DNA, 24 bp, Artificial Sequence
  source: /note="Description of Artificial Sequence: Syntheticoligonucleotide"
aacagtcgcgaattataaatacgg

SEQ ID NO: 957 - DNA, 24 bp, Artificial Sequence
  source: /note="Description of Artificial Sequence: Syntheticoligonucleotide"
atcacacacatacacagtgagctc

SEQ ID NO: 958 - DNA, 24 bp, Artificial Sequence
  source: /note="Description of Artificial Sequence: Syntheticoligonucleotide"
catcacccttcgcatctcggacca

SEQ ID NO: 959 - DNA, 24 bp, Artificial Sequence
  source: /note="Description of Artificial Sequence: Syntheticoligonucleotide"
gcgcctgtccctcttacagtttct

SEQ ID NO: 960 - DNA, 24 bp, Artificial Sequence
  source: /note="Description of Artificial Sequence: Syntheticoligonucleotide"
tacctgtattactttactggcaac

SEQ ID NO: 961 - DNA, 24 bp, Artificial Sequence
  source: /note="Description of Artificial Sequence: Syntheticoligonucleotide"
tgatccgtcacacaatccttcttg

SEQ ID NO: 962 - DNA, 24 bp, Artificial Sequence
  source: /note="Description of Artificial Sequence: Syntheticoligonucleotide"
ttgaatacaaccaatacttatcgg

SEQ ID NO: 963 - DNA, 24 bp, Artificial Sequence
  source: /note="Description of Artificial Sequence: Syntheticoligonucleotide"
gagcagtacgcttcaactcaattc

SEQ ID NO: 964 - DNA, 24 bp, Artificial Sequence
  source: /note="Description of Artificial Sequence: Syntheticoligonucleotide"
gttggaacgccaaatcgtctgaac

SEQ ID NO: 965 - DNA, 24 bp, Artificial Sequence
  source: /note="Description of Artificial Sequence: Syntheticoligonucleotide"
ctttcaacatcgaatccttgctgg

SEQ ID NO: 966 - DNA, 24 bp, Artificial Sequence
  source: /note="Description of Artificial Sequence: Syntheticoligonucleotide"
gatgcttcttaggaattgtgcagt

SEQ ID NO: 967 - DNA, 24 bp, Artificial Sequence
  source: /note="Description of Artificial Sequence: Syntheticoligonucleotide"
acgaaatttacgattaggtcagcg

SEQ ID NO: 968 - DNA, 24 bp, Artificial Sequence
  source: /note="Description of Artificial Sequence: Syntheticoligonucleotide"
acaggaacccgctctccgcaggac

SEQ ID NO: 969 - DNA, 24 bp, Artificial Sequence
  source: /note="Description of Artificial Sequence: Syntheticoligonucleotide"
attcagaaatgcgtctgttgacgg

SEQ ID NO: 970 - DNA, 24 bp, Artificial Sequence
  source: /note="Description of Artificial Sequence: Syntheticoligonucleotide"
cgcgaatataatagcaagtagttg

SEQ ID NO: 971 - DNA, 24 bp, Artificial Sequence
  source: /note="Description of Artificial Sequence: Syntheticoligonucleotide"
atgagttagttaggcacacatcat

SEQ ID NO: 972 - DNA, 24 bp, Artificial Sequence
  source: /note="Description of Artificial Sequence: Syntheticoligonucleotide"
ctaggcttgtaacagattggctag

SEQ ID NO: 973 - DNA, 24 bp, Artificial Sequence
  source: /note="Description of Artificial Sequence: Syntheticoligonucleotide"
tcaatctatgtgacactgtccacc

SEQ ID NO: 974 - DNA, 24 bp, Artificial Sequence
  source: /note="Description of Artificial Sequence: Syntheticoligonucleotide"
gctcaacataagacgttactcagc

SEQ ID NO: 975 - DNA, 24 bp, Artificial Sequence
  source: /note="Description of Artificial Sequence: Syntheticoligonucleotide"
aacgtaacgacatgagtgtccctg

SEQ ID NO: 976 - DNA, 24 bp, Artificial Sequence
  source: /note="Description of Artificial Sequence: Syntheticoligonucleotide"
ccgacattcctgcctagttccgcg

SEQ ID NO: 977 - DNA, 24 bp, Artificial Sequence
  source: /note="Description of Artificial Sequence: Syntheticoligonucleotide"
ctagcgtacgtcgctataggtctt

SEQ ID NO: 978 - DNA, 24 bp, Artificial Sequence
  source: /note="Description of Artificial Sequence: Syntheticoligonucleotide"
tgccgcgtataaatcaactgactg

SEQ ID NO: 979 - DNA, 24 bp, Artificial Sequence
  source: /note="Description of Artificial Sequence: Syntheticoligonucleotide"
ttacacaagacaccttctcctaca

SEQ ID NO: 980 - DNA, 24 bp, Artificial Sequence
  source: /note="Description of Artificial Sequence: Syntheticoligonucleotide"
ccaccctgccttatcttcgtgtat

SEQ ID NO: 981 - DNA, 24 bp, Artificial Sequence
  source: /note="Description of Artificial Sequence: Syntheticoligonucleotide"
aaattagctgcgcgtgtacatacg

SEQ ID NO: 982 - DNA, 24 bp, Artificial Sequence
  source: /note="Description of Artificial Sequence: Syntheticoligonucleotide"
gtcccgcgaccttcaatgagtcgt

SEQ ID NO: 983 - DNA, 24 bp, Artificial Sequence
  source: /note="Description of Artificial Sequence: Syntheticoligonucleotide"
acatgttattcctccgtaagcttg

SEQ ID NO: 984 - DNA, 24 bp, Artificial Sequence
  source: /note="Description of Artificial Sequence: Syntheticoligonucleotide"
gtcaagggccgtgcgtaattcggg

SEQ ID NO: 985 - DNA, 24 bp, Artificial Sequence
  source: /note="Description of Artificial Sequence: Syntheticoligonucleotide"
cctaagttaacatcagctatgtac

SEQ ID NO: 986 - DNA, 24 bp, Artificial Sequence
  source: /note="Description of Artificial Sequence: Syntheticoligonucleotide"
gagtggtaattcatcttggcctca

SEQ ID NO: 987 - DNA, 24 bp, Artificial Sequence
  source: /note="Description of Artificial Sequence: Syntheticoligonucleotide"
catatctaaagcagacggacataa

SEQ ID NO: 988 - DNA, 24 bp, Artificial Sequence
  source: /note="Description of Artificial Sequence: Syntheticoligonucleotide"
gatcctcgagccggtcgaacttac

SEQ ID NO: 989 - DNA, 24 bp, Artificial Sequence
  source: /note="Description of Artificial Sequence: Syntheticoligonucleotide"
gtctttcgacgcttatcaaactac

SEQ ID NO: 990 - DNA, 24 bp, Artificial Sequence
  source: /note="Description of Artificial Sequence: Syntheticoligonucleotide"
acccataagatagcgcgcattcgt

SEQ ID NO: 991 - DNA, 24 bp, Artificial Sequence
  source: /note="Description of Artificial Sequence: Syntheticoligonucleotide"
ctgggtcttaatgcggaggttgga

SEQ ID NO: 992 - DNA, 24 bp, Artificial Sequence
  source: /note="Description of Artificial Sequence: Syntheticoligonucleotide"
acatgaggccggtgggatttgaaa

SEQ ID NO: 993 - DNA, 24 bp, Artificial Sequence
  source: /note="Description of Artificial Sequence: Syntheticoligonucleotide"
taaccgctgccctgataaagaatt

SEQ ID NO: 994 - DNA, 24 bp, Artificial Sequence
  source: /note="Description of Artificial Sequence: Syntheticoligonucleotide"
agatccgggcctctctatcatagt

SEQ ID NO: 995 - DNA, 24 bp, Artificial Sequence
  source: /note="Description of Artificial Sequence: Syntheticoligonucleotide"
agtacgtatcaaaggaatgatcaa

SEQ ID NO: 996 - DNA, 24 bp, Artificial Sequence
  source: /note="Description of Artificial Sequence: Syntheticoligonucleotide"
tccagatcctctgtctttgcataa

SEQ ID NO: 997 - DNA, 24 bp, Artificial Sequence
  source: /note="Description of Artificial Sequence: Syntheticoligonucleotide"
tgagtctttccgcttagctactgg

SEQ ID NO: 998 - DNA, 24 bp, Artificial Sequence
  source: /note="Description of Artificial Sequence: Syntheticoligonucleotide"
agccattcaattggtatacggagt

SEQ ID NO: 999 - DNA, 24 bp, Artificial Sequence
  source: /note="Description of Artificial Sequence: Syntheticoligonucleotide"
tgcactatttgtaggtaggtatgc

SEQ ID NO: 1000 - DNA, 24 bp, Artificial Sequence
  source: /note="Description of Artificial Sequence: Syntheticoligonucleotide"
gattaaatagctttgccggttgtg

SEQ ID NO: 1001 - DNA, 24 bp, Artificial Sequence
  source: /note="Description of Artificial Sequence: Syntheticoligonucleotide"
ttcgcacaaagtactgctcgtccc

SEQ ID NO: 1002 - DNA, 24 bp, Artificial Sequence
  source: /note="Description of Artificial Sequence: Syntheticoligonucleotide"
aattacgactaaagctctcgcggc

SEQ ID NO: 1003 - DNA, 24 bp, Artificial Sequence
  source: /note="Description of Artificial Sequence: Syntheticoligonucleotide"
tttcagctctcattgggcattgca

SEQ ID NO: 1004 - DNA, 24 bp, Artificial Sequence
  source: /note="Description of Artificial Sequence: Syntheticoligonucleotide"
agactctattcctgaggacctgac

SEQ ID NO: 1005 - DNA, 24 bp, Artificial Sequence
  source: /note="Description of Artificial Sequence: Syntheticoligonucleotide"
agtccaatcactttccttcactcc

SEQ ID NO: 1006 - DNA, 24 bp, Artificial Sequence
  source: /note="Description of Artificial Sequence: Syntheticoligonucleotide"
accgcaacgactcgctttctcatt

SEQ ID NO: 1007 - DNA, 24 bp, Artificial Sequence
  source: /note="Description of Artificial Sequence: Syntheticoligonucleotide"
atcccggacggctatgtcaagcgg

SEQ ID NO: 1008 - DNA, 24 bp, Artificial Sequence
  source: /note="Description of Artificial Sequence: Syntheticoligonucleotide"
catgtgttgatcggagaccgctga

SEQ ID NO: 1009 - DNA, 24 bp, Artificial Sequence
  source: /note="Description of Artificial Sequence: Syntheticoligonucleotide"
gtatatcatccgacgtgtctatct

SEQ ID NO: 1010 - DNA, 24 bp, Artificial Sequence
  source: /note="Description of Artificial Sequence: Syntheticoligonucleotide"
tcaactgcagcgagcgaattcctt

SEQ ID NO: 1011 - DNA, 24 bp, Artificial Sequence
  source: /note="Description of Artificial Sequence: Syntheticoligonucleotide"
ttgtataggtccctccactaggta

SEQ ID NO: 1012 - DNA, 24 bp, Artificial Sequence
  source: /note="Description of Artificial Sequence: Syntheticoligonucleotide"
tacccgattcttatcaaactctac

SEQ ID NO: 1013 - DNA, 24 bp, Artificial Sequence
  source: /note="Description of Artificial Sequence: Syntheticoligonucleotide"
gtctagaaccatcatcaacctgtc

SEQ ID NO: 1014 - DNA, 24 bp, Artificial Sequence
  source: /note="Description of Artificial Sequence: Syntheticoligonucleotide"
atcagggtcaatgccatggaaggt

SEQ ID NO: 1015 - DNA, 24 bp, Artificial Sequence
  source: /note="Description of Artificial Sequence: Syntheticoligonucleotide"
ccatgtgaacgcataggctgcgaa

SEQ ID NO: 1016 - DNA, 24 bp, Artificial Sequence
  source: /note="Description of Artificial Sequence: Syntheticoligonucleotide"
ttcacctgttgccgctgtatatat

SEQ ID NO: 1017 - DNA, 24 bp, Artificial Sequence
  source: /note="Description of Artificial Sequence: Syntheticoligonucleotide"
cgcccgagtattgcttaggccgac

SEQ ID NO: 1018 - DNA, 24 bp, Artificial Sequence
  source: /note="Description of Artificial Sequence: Syntheticoligonucleotide"
ggcgttgtgtcgggtatcgtcgat

SEQ ID NO: 1019 - DNA, 24 bp, Artificial Sequence
  source: /note="Description of Artificial Sequence: Syntheticoligonucleotide"
tggacgaataaacattgctagccc

SEQ ID NO: 1020 - DNA, 24 bp, Artificial Sequence
  source: /note="Description of Artificial Sequence: Syntheticoligonucleotide"
ttgaagctacagacatgtgcatga

SEQ ID NO: 1021 - DNA, 24 bp, Artificial Sequence
  source: /note="Description of Artificial Sequence: Syntheticoligonucleotide"
tacttactgtctggatggacaggt

SEQ ID NO: 1022 - DNA, 24 bp, Artificial Sequence
  source: /note="Description of Artificial Sequence: Syntheticoligonucleotide"
gatgacaccaatctggctagactc

SEQ ID NO: 1023 - DNA, 24 bp, Artificial Sequence
  source: /note="Description of Artificial Sequence: Syntheticoligonucleotide"
cttggagccttgatctagaatgaa

SEQ ID NO: 1024 - DNA, 24 bp, Artificial Sequence
  source: /note="Description of Artificial Sequence: Syntheticoligonucleotide"
accttagaactttccagtgagtgg

SEQ ID NO: 1025 - DNA, 24 bp, Artificial Sequence
  source: /note="Description of Artificial Sequence: Syntheticoligonucleotide"
gttctggttcgtcagtcgcctaaa

SEQ ID NO: 1026 - DNA, 24 bp, Artificial Sequence
  source: /note="Description of Artificial Sequence: Syntheticoligonucleotide"
ctacatccgaggagcaagatacaa

SEQ ID NO: 1027 - DNA, 24 bp, Artificial Sequence
  source: /note="Description of Artificial Sequence: Syntheticoligonucleotide"
gtaatgaggacacaatcgatcagt

SEQ ID NO: 1028 - DNA, 24 bp, Artificial Sequence
  source: /note="Description of Artificial Sequence: Syntheticoligonucleotide"
caagttagcactgaaggctacaca

SEQ ID NO: 1029 - DNA, 24 bp, Artificial Sequence
  source: /note="Description of Artificial Sequence: Syntheticoligonucleotide"
accgaccttgtgagtcctaaacat

SEQ ID NO: 1030 - DNA, 24 bp, Artificial Sequence
  source: /note="Description of Artificial Sequence: Syntheticoligonucleotide"
gagaatggtgatccgtgtgattat

SEQ ID NO: 1031 - DNA, 24 bp, Artificial Sequence
  source: /note="Description of Artificial Sequence: Syntheticoligonucleotide"
gttaatcgtatgcggcatgaacaa

SEQ ID NO: 1032 - DNA, 24 bp, Artificial Sequence
  source: /note="Description of Artificial Sequence: Syntheticoligonucleotide"
aactcaatctcgtgacagcataca

SEQ ID NO: 1033 - DNA, 24 bp, Artificial Sequence
  source: /note="Description of Artificial Sequence: Syntheticoligonucleotide"
tggtcaaaccttcacaaacaactc

SEQ ID NO: 1034 - DNA, 24 bp, Artificial Sequence
  source: /note="Description of Artificial Sequence: Syntheticoligonucleotide"
tcgacgaacttacacttggtcgta

SEQ ID NO: 1035 - DNA, 24 bp, Artificial Sequence
  source: /note="Description of Artificial Sequence: Syntheticoligonucleotide"
tacatgtcctcttaccacccttca

SEQ ID NO: 1036 - DNA, 24 bp, Artificial Sequence
  source: /note="Description of Artificial Sequence: Syntheticoligonucleotide"
gctttggatgctcaatatacacta

SEQ ID NO: 1037 - DNA, 24 bp, Artificial Sequence
  source: /note="Description of Artificial Sequence: Syntheticoligonucleotide"
cagagaaacgaggttcaggatctc

SEQ ID NO: 1038 - DNA, 24 bp, Artificial Sequence
  source: /note="Description of Artificial Sequence: Syntheticoligonucleotide"
attgatgtggcacccaaacgcaag

SEQ ID NO: 1039 - DNA, 24 bp, Artificial Sequence
  source: /note="Description of Artificial Sequence: Syntheticoligonucleotide"
ctttcttaggttgcatcaatttag

SEQ ID NO: 1040 - DNA, 24 bp, Artificial Sequence
  source: /note="Description of Artificial Sequence: Syntheticoligonucleotide"
gatgcacgtcattagtctactata

SEQ ID NO: 1041 - DNA, 24 bp, Artificial Sequence
  source: /note="Description of Artificial Sequence: Syntheticoligonucleotide"
gattcgggaaacagactgtgcttc

SEQ ID NO: 1042 - DNA, 24 bp, Artificial Sequence
  source: /note="Description of Artificial Sequence: Syntheticoligonucleotide"
tagtaacgtaacacagtttaatta

SEQ ID NO: 1043 - DNA, 24 bp, Artificial Sequence
  source: /note="Description of Artificial Sequence: Syntheticoligonucleotide"
acaaccgcagttaagataacacta

SEQ ID NO: 1044 - DNA, 24 bp, Artificial Sequence
  source: /note="Description of Artificial Sequence: Syntheticoligonucleotide"
caccgacatgagatataacataga

SEQ ID NO: 1045 - DNA, 24 bp, Artificial Sequence
  source: /note="Description of Artificial Sequence: Syntheticoligonucleotide"
ttcgtaacaacaacggcgtttcgt

SEQ ID NO: 1046 - DNA, 24 bp, Artificial Sequence
  source: /note="Description of Artificial Sequence: Syntheticoligonucleotide"
gtgactgggtttggaattatgctt

SEQ ID NO: 1047 - DNA, 24 bp, Artificial Sequence
  source: /note="Description of Artificial Sequence: Syntheticoligonucleotide"
ggtgcgtgtagcatgagaattatc

SEQ ID NO: 1048 - DNA, 24 bp, Artificial Sequence
  source: /note="Description of Artificial Sequence: Syntheticoligonucleotide"
acgtaacgaactatcaatgcggtt

SEQ ID NO: 1049 - DNA, 24 bp, Artificial Sequence
  source: /note="Description of Artificial Sequence: Syntheticoligonucleotide"
atcccagaacggagcctggcccaa

SEQ ID NO: 1050 - DNA, 24 bp, Artificial Sequence
  source: /note="Description of Artificial Sequence: Syntheticoligonucleotide"
catcttgcactaagtcaggaagca

SEQ ID NO: 1051 - DNA, 24 bp, Artificial Sequence
  source: /note="Description of Artificial Sequence: Syntheticoligonucleotide"
gtgattccactttataggacacgg

SEQ ID NO: 1052 - DNA, 24 bp, Artificial Sequence
  source: /note="Description of Artificial Sequence: Syntheticoligonucleotide"
gtgaacagcaggagaaccaatacg

SEQ ID NO: 1053 - DNA, 24 bp, Artificial Sequence
  source: /note="Description of Artificial Sequence: Syntheticoligonucleotide"
agggtttgtgaatctaggagcaca

SEQ ID NO: 1054 - DNA, 24 bp, Artificial Sequence
  source: /note="Description of Artificial Sequence: Syntheticoligonucleotide"
gggagcaataaacataaccatcca

SEQ ID NO: 1055 - DNA, 24 bp, Artificial Sequence
  source: /note="Description of Artificial Sequence: Syntheticoligonucleotide"
ttccatttggatcatttcggaccg

SEQ ID NO: 1056 - DNA, 24 bp, Artificial Sequence
  source: /note="Description of Artificial Sequence: Syntheticoligonucleotide"
tggcccatactcgtttactgggtg

SEQ ID NO: 1057 - DNA, 24 bp, Artificial Sequence
  source: /note="Description of Artificial Sequence: Syntheticoligonucleotide"
tccaccacagagtacgacgatttg

SEQ ID NO: 1058 - DNA, 24 bp, Artificial Sequence
  source: /note="Description of Artificial Sequence: Syntheticoligonucleotide"
ttatattcgctcagtacgattgac

SEQ ID NO: 1059 - DNA, 24 bp, Artificial Sequence
  source: /note="Description of Artificial Sequence: Syntheticoligonucleotide"
gaccgtttagatgtttcagaacag

SEQ ID NO: 1060 - DNA, 24 bp, Artificial Sequence
  source: /note="Description of Artificial Sequence: Syntheticoligonucleotide"
gatacgtcgtaaacctagatagtc

SEQ ID NO: 1061 - DNA, 24 bp, Artificial Sequence
  source: /note="Description of Artificial Sequence: Syntheticoligonucleotide"
tatttgtcacgtcctgtatgaccg

SEQ ID NO: 1062 - DNA, 24 bp, Artificial Sequence
  source: /note="Description of Artificial Sequence: Syntheticoligonucleotide"
cacctgtccttgtggtttgcctaa

SEQ ID NO: 1063 - DNA, 24 bp, Artificial Sequence
  source: /note="Description of Artificial Sequence: Syntheticoligonucleotide"
cagagccggtagatgtatggcata

SEQ ID NO: 1064 - DNA, 24 bp, Artificial Sequence
  source: /note="Description of Artificial Sequence: Syntheticoligonucleotide"
tagggacggtgtgtagccgagcta

SEQ ID NO: 1065 - DNA, 24 bp, Artificial Sequence
  source: /note="Description of Artificial Sequence: Syntheticoligonucleotide"
tgatttggaggacgagacgcgcat

SEQ ID NO: 1066 - DNA, 24 bp, Artificial Sequence
  source: /note="Description of Artificial Sequence: Syntheticoligonucleotide"
gctattcgcagtttccacggaact

SEQ ID NO: 1067 - DNA, 24 bp, Artificial Sequence
  source: /note="Description of Artificial Sequence: Syntheticoligonucleotide"
tgtgcagaatagcggcatcgtctt

SEQ ID NO: 1068 - DNA, 24 bp, Artificial Sequence
  source: /note="Description of Artificial Sequence: Syntheticoligonucleotide"
tacgggctcgtcttaacgggaatt

SEQ ID NO: 1069 - DNA, 24 bp, Artificial Sequence
  source: /note="Description of Artificial Sequence: Syntheticoligonucleotide"
ctgacagacgttattgtctacaca

SEQ ID NO: 1070 - DNA, 24 bp, Artificial Sequence
  source: /note="Description of Artificial Sequence: Syntheticoligonucleotide"
gcccatctacacttagctatagaa

SEQ ID NO: 1071 - DNA, 24 bp, Artificial Sequence
  source: /note="Description of Artificial Sequence: Syntheticoligonucleotide"
aaccgcacatagtaaatagctcaa

SEQ ID NO: 1072 - DNA, 24 bp, Artificial Sequence
  source: /note="Description of Artificial Sequence: Syntheticoligonucleotide"
gtgagcagcagctaatacctgtaa

SEQ ID NO: 1073 - DNA, 24 bp, Artificial Sequence
  source: /note="Description of Artificial Sequence: Syntheticoligonucleotide"
tacgtatacctgggatgaacagac

SEQ ID NO: 1074 - DNA, 24 bp, Artificial Sequence
  source: /note="Description of Artificial Sequence: Syntheticoligonucleotide"
tttctatgagtttgaacaacgtcg

SEQ ID NO: 1075 - DNA, 24 bp, Artificial Sequence
  source: /note="Description of Artificial Sequence: Syntheticoligonucleotide"
aactgtctgtaagcacccaaggat

SEQ ID NO: 1076 - DNA, 24 bp, Artificial Sequence
  source: /note="Description of Artificial Sequence: Syntheticoligonucleotide"
ggaaagtgtctcggccgtactttc

SEQ ID NO: 1077 - DNA, 24 bp, Artificial Sequence
  source: /note="Description of Artificial Sequence: Syntheticoligonucleotide"
acccacaaatatagggctgtcttg

SEQ ID NO: 1078 - DNA, 24 bp, Artificial Sequence
  source: /note="Description of Artificial Sequence: Syntheticoligonucleotide"
gcgaaaccagtctttactttggcc

SEQ ID NO: 1079 - DNA, 24 bp, Artificial Sequence
  source: /note="Description of Artificial Sequence: Syntheticoligonucleotide"
gatccaccatttcttgaactgcaa

SEQ ID NO: 1080 - DNA, 24 bp, Artificial Sequence
  source: /note="Description of Artificial Sequence: Syntheticoligonucleotide"
taacactgggcgtcataggattgc

SEQ ID NO: 1081 - DNA, 24 bp, Artificial Sequence
  source: /note="Description of Artificial Sequence: Syntheticoligonucleotide"
gcttcaacctggactgtgctgtta

SEQ ID NO: 1082 - DNA, 24 bp, Artificial Sequence
  source: /note="Description of Artificial Sequence: Syntheticoligonucleotide"
ttgcgattactacaaagagtagcc

SEQ ID NO: 1083 - DNA, 24 bp, Artificial Sequence
  source: /note="Description of Artificial Sequence: Syntheticoligonucleotide"
cggagtttgccactgcttcctatg

SEQ ID NO: 1084 - DNA, 24 bp, Artificial Sequence
  source: /note="Description of Artificial Sequence: Syntheticoligonucleotide"
tggacgcatggagagtgggtatcc

SEQ ID NO: 1085 - DNA, 24 bp, Artificial Sequence
  source: /note="Description of Artificial Sequence: Syntheticoligonucleotide"
agatgcaatagcgttggctgaatg

SEQ ID NO: 1086 - DNA, 24 bp, Artificial Sequence
  source: /note="Description of Artificial Sequence: Syntheticoligonucleotide"
tacattatccgagtgggcgaggtt

SEQ ID NO: 1087 - DNA, 24 bp, Artificial Sequence
  source: /note="Description of Artificial Sequence: Syntheticoligonucleotide"
tgattgtgcaacccgtaaccttta

SEQ ID NO: 1088 - DNA, 24 bp, Artificial Sequence
  source: /note="Description of Artificial Sequence: Syntheticoligonucleotide"
acataagaagagcatgcactcttg

SEQ ID NO: 1089 - DNA, 24 bp, Artificial Sequence
  source: /note="Description of Artificial Sequence: Syntheticoligonucleotide"
tgatgagaacatatagcccacagg

SEQ ID NO: 1090 - DNA, 24 bp, Artificial Sequence
  source: /note="Description of Artificial Sequence: Syntheticoligonucleotide"
gtgctacgccttcacttaagctat

SEQ ID NO: 1091 - DNA, 24 bp, Artificial Sequence
  source: /note="Description of Artificial Sequence: Syntheticoligonucleotide"
gagaccatttgtaatcacatcgcc

SEQ ID NO: 1092 - DNA, 24 bp, Artificial Sequence
  source: /note="Description of Artificial Sequence: Syntheticoligonucleotide"
aataagattcagctggagtagagc

SEQ ID NO: 1093 - DNA, 24 bp, Artificial Sequence
  source: /note="Description of Artificial Sequence: Syntheticoligonucleotide"
taatgggtcattagattcgaagga

SEQ ID NO: 1094 - DNA, 24 bp, Artificial Sequence
  source: /note="Description of Artificial Sequence: Syntheticoligonucleotide"
atgctttgtccactattaacgtcg

SEQ ID NO: 1095 - DNA, 24 bp, Artificial Sequence
  source: /note="Description of Artificial Sequence: Syntheticoligonucleotide"
gataactgatttgctttcgggagt

SEQ ID NO: 1096 - DNA, 24 bp, Artificial Sequence
  source: /note="Description of Artificial Sequence: Syntheticoligonucleotide"
actctatcgggttgggtagttctt

SEQ ID NO: 1097 - DNA, 24 bp, Artificial Sequence
  source: /note="Description of Artificial Sequence: Syntheticoligonucleotide"
atggctgtaggacatagttgtaag

SEQ ID NO: 1098 - DNA, 24 bp, Artificial Sequence
  source: /note="Description of Artificial Sequence: Syntheticoligonucleotide"
aggtacgctaacaggacaaatcca

SEQ ID NO: 1099 - DNA, 24 bp, Artificial Sequence
  source: /note="Description of Artificial Sequence: Syntheticoligonucleotide"
catatctgttaagtcattcctccg

SEQ ID NO: 1100 - DNA, 24 bp, Artificial Sequence
  source: /note="Description of Artificial Sequence: Syntheticoligonucleotide"
cctcggagtagttggatcctgatg

SEQ ID NO: 1101 - DNA, 24 bp, Artificial Sequence
  source: /note="Description of Artificial Sequence: Syntheticoligonucleotide"
taaacattgcgggaagcttaacta

SEQ ID NO: 1102 - DNA, 24 bp, Artificial Sequence
  source: /note="Description of Artificial Sequence: Syntheticoligonucleotide"
ggttaatccctaatagatctcact

SEQ ID NO: 1103 - DNA, 24 bp, Artificial Sequence
  source: /note="Description of Artificial Sequence: Syntheticoligonucleotide"
caaattgctggcgttggtaatctg

SEQ ID NO: 1104 - DNA, 24 bp, Artificial Sequence
  source: /note="Description of Artificial Sequence: Syntheticoligonucleotide"
cggctgtcaatatgtgaattccgc

SEQ ID NO: 1105 - DNA, 24 bp, Artificial Sequence
  source: /note="Description of Artificial Sequence: Syntheticoligonucleotide"
tgcagtcggtgctctcttactcta

SEQ ID NO: 1106 - DNA, 24 bp, Artificial Sequence
  source: /note="Description of Artificial Sequence: Syntheticoligonucleotide"
ttcccattgatccgccgagcatta

SEQ ID NO: 1107 - DNA, 24 bp, Artificial Sequence
  source: /note="Description of Artificial Sequence: Syntheticoligonucleotide"
tcggtagcacagcagaccttaggt

SEQ ID NO: 1108 - DNA, 24 bp, Artificial Sequence
  source: /note="Description of Artificial Sequence: Syntheticoligonucleotide"
atcatcgcgctttcaaacgggtta

SEQ ID NO: 1109 - DNA, 24 bp, Artificial Sequence
  source: /note="Description of Artificial Sequence: Syntheticoligonucleotide"
tcttgtctgggagctagcaaattc

SEQ ID NO: 1110 - DNA, 24 bp, Artificial Sequence
  source: /note="Description of Artificial Sequence: Syntheticoligonucleotide"
taaagtgttggtatggccctctaa

SEQ ID NO: 1111 - DNA, 24 bp, Artificial Sequence
  source: /note="Description of Artificial Sequence: Syntheticoligonucleotide"
atggatccgtgccgaatcagatcg

SEQ ID NO: 1112 - DNA, 24 bp, Artificial Sequence
  source: /note="Description of Artificial Sequence: Syntheticoligonucleotide"
gagtatcggcacttccacatcctg

SEQ ID NO: 1113 - DNA, 24 bp, Artificial Sequence
  source: /note="Description of Artificial Sequence: Syntheticoligonucleotide"
tagcttggatctcgtgcaattagg

SEQ ID NO: 1114 - DNA, 24 bp, Artificial Sequence
  source: /note="Description of Artificial Sequence: Syntheticoligonucleotide"
ccgtcgctcacatttcctggagac

SEQ ID NO: 1115 - DNA, 24 bp, Artificial Sequence
  source: /note="Description of Artificial Sequence: Syntheticoligonucleotide"
cgaaattaggcggatgctacggga

SEQ ID NO: 1116 - DNA, 24 bp, Artificial Sequence
  source: /note="Description of Artificial Sequence: Syntheticoligonucleotide"
tgagtgtgttaatgatgtctcgat

SEQ ID NO: 1117 - DNA, 24 bp, Artificial Sequence
  source: /note="Description of Artificial Sequence: Syntheticoligonucleotide"
acccaatggtcaatcagcacagag

SEQ ID NO: 1118 - DNA, 24 bp, Artificial Sequence
  source: /note="Description of Artificial Sequence: Syntheticoligonucleotide"
ttactggtttacgttcgggaggta

SEQ ID NO: 1119 - DNA, 24 bp, Artificial Sequence
  source: /note="Description of Artificial Sequence: Syntheticoligonucleotide"
tcctcgataatcacatcgctttcc

SEQ ID NO: 1120 - DNA, 24 bp, Artificial Sequence
  source: /note="Description of Artificial Sequence: Syntheticoligonucleotide"
aagacaacggtggcgtagtcaaat

SEQ ID NO: 1121 - DNA, 24 bp, Artificial Sequence
  source: /note="Description of Artificial Sequence: Syntheticoligonucleotide"
gtcgccgtagataagtactgtaca

SEQ ID NO: 1122 - DNA, 24 bp, Artificial Sequence
  source: /note="Description of Artificial Sequence: Syntheticoligonucleotide"
gggtgtccctcaacctcagatttg

SEQ ID NO: 1123 - DNA, 24 bp, Artificial Sequence
  source: /note="Description of Artificial Sequence: Syntheticoligonucleotide"
tgttgcctggtctctcacgtatat

SEQ ID NO: 1124 - DNA, 24 bp, Artificial Sequence
  source: /note="Description of Artificial Sequence: Syntheticoligonucleotide"
tcagctgcctaaatggcagtgagc

SEQ ID NO: 1125 - DNA, 24 bp, Artificial Sequence
  source: /note="Description of Artificial Sequence: Syntheticoligonucleotide"
atcgtcaggagacaaccagagacg

SEQ ID NO: 1126 - DNA, 24 bp, Artificial Sequence
  source: /note="Description of Artificial Sequence: Syntheticoligonucleotide"
aagttaccatgctggtaggctgag

SEQ ID NO: 1127 - DNA, 24 bp, Artificial Sequence
  source: /note="Description of Artificial Sequence: Syntheticoligonucleotide"
agacactccttaagaggaaggctc

SEQ ID NO: 1128 - DNA, 24 bp, Artificial Sequence
  source: /note="Description of Artificial Sequence: Syntheticoligonucleotide"
acagcaaattacagagggtcgcaa

SEQ ID NO: 1129 - DNA, 24 bp, Artificial Sequence
  source: /note="Description of Artificial Sequence: Syntheticoligonucleotide"
tacgatagggctgctacttgccaa

SEQ ID NO: 1130 - DNA, 24 bp, Artificial Sequence
  source: /note="Description of Artificial Sequence: Syntheticoligonucleotide"
ttgatggataagctcacaccagaa

SEQ ID NO: 1131 - DNA, 24 bp, Artificial Sequence
  source: /note="Description of Artificial Sequence: Syntheticoligonucleotide"
attggttcgcccgagaaatatccg

SEQ ID NO: 1132 - DNA, 24 bp, Artificial Sequence
  source: /note="Description of Artificial Sequence: Syntheticoligonucleotide"
tgacggtatcacggctgcgcatct

SEQ ID NO: 1133 - DNA, 24 bp, Artificial Sequence
  source: /note="Description of Artificial Sequence: Syntheticoligonucleotide"
gatctgtggttcataggtgtccgt

SEQ ID NO: 1134 - DNA, 24 bp, Artificial Sequence
  source: /note="Description of Artificial Sequence: Syntheticoligonucleotide"
tgcaaacgaaagtcaattagcctc

SEQ ID NO: 1135 - DNA, 24 bp, Artificial Sequence
  source: /note="Description of Artificial Sequence: Syntheticoligonucleotide"
gtatttcacaaacctctaccatcc

SEQ ID NO: 1136 - DNA, 24 bp, Artificial Sequence
  source: /note="Description of Artificial Sequence: Syntheticoligonucleotide"
aattcagcccatctgctacgccta

SEQ ID NO: 1137 - DNA, 24 bp, Artificial Sequence
  source: /note="Description of Artificial Sequence: Syntheticoligonucleotide"
atggcctaaacccataagagactc

SEQ ID NO: 1138 - DNA, 24 bp, Artificial Sequence
  source: /note="Description of Artificial Sequence: Syntheticoligonucleotide"
gacgccctttcgccaagccattga

SEQ ID NO: 1139 - DNA, 24 bp, Artificial Sequence
  source: /note="Description of Artificial Sequence: Syntheticoligonucleotide"
gaaccgagtcacgggcaaagtata

SEQ ID NO: 1140 - DNA, 24 bp, Artificial Sequence
  source: /note="Description of Artificial Sequence: Syntheticoligonucleotide"
atccatttattgctcattagcgtg

SEQ ID NO: 1141 - DNA, 24 bp, Artificial Sequence
  source: /note="Description of Artificial Sequence: Syntheticoligonucleotide"
cgcttggaccggaaatcatgggtt

SEQ ID NO: 1142 - DNA, 24 bp, Artificial Sequence
  source: /note="Description of Artificial Sequence: Syntheticoligonucleotide"
ttgtcagcgcttaatatttatgcc

SEQ ID NO: 1143 - DNA, 24 bp, Artificial Sequence
  source: /note="Description of Artificial Sequence: Syntheticoligonucleotide"
tagtgtgtgtatgtgtcactcgag

SEQ ID NO: 1144 - DNA, 24 bp, Artificial Sequence
  source: /note="Description of Artificial Sequence: Syntheticoligonucleotide"
gtagtgggaagcgtagagcctggt

SEQ ID NO: 1145 - DNA, 24 bp, Artificial Sequence
  source: /note="Description of Artificial Sequence: Syntheticoligonucleotide"
cgcggacagggagaatgtcaaaga

SEQ ID NO: 1146 - DNA, 24 bp, Artificial Sequence
  source: /note="Description of Artificial Sequence: Syntheticoligonucleotide"
atggacataatgaaatgaccgttg

SEQ ID NO: 1147 - DNA, 24 bp, Artificial Sequence
  source: /note="Description of Artificial Sequence: Syntheticoligonucleotide"
actaggcagtgtgttaggaagaac

SEQ ID NO: 1148 - DNA, 24 bp, Artificial Sequence
  source: /note="Description of Artificial Sequence: Syntheticoligonucleotide"
aacttatgttggttatgaatagcc

SEQ ID NO: 1149 - DNA, 24 bp, Artificial Sequence
  source: /note="Description of Artificial Sequence: Syntheticoligonucleotide"
ctcgtcatgcgaagttgagttaag

SEQ ID NO: 1150 - DNA, 24 bp, Artificial Sequence
  source: /note="Description of Artificial Sequence: Syntheticoligonucleotide"
caaccttgcggtttagcagacttg

SEQ ID NO: 1151 - DNA, 24 bp, Artificial Sequence
  source: /note="Description of Artificial Sequence: Syntheticoligonucleotide"
gaaagttgtagcttaggaacgacc

SEQ ID NO: 1152 - DNA, 24 bp, Artificial Sequence
  source: /note="Description of Artificial Sequence: Syntheticoligonucleotide"
ctacgaagaatccttaacacgtca

SEQ ID NO: 1153 - DNA, 24 bp, Artificial Sequence
  source: /note="Description of Artificial Sequence: Syntheticoligonucleotide"
tgctttaaatgctaatccagtcgc

SEQ ID NO: 1154 - DNA, 24 bp, Artificial Sequence
  source: /note="Description of Artificial Sequence: Syntheticoligonucleotide"
tgtccttgggcgagaggcgtcctg

SEQ ID NO: 1155 - DNA, 24 bp, Artificial Sequence
  source: /note="Description of Artificial Sequence: Syntheticoligonucleotide"
taagtctttacgcagacaactgcc

SEQ ID NO: 1156 - DNA, 24 bp, Artificial Sequence
  source: /note="Description of Artificial Sequence: Syntheticoligonucleotide"
gcgcttatattatcgttcatcaac

SEQ ID NO: 1157 - DNA, 24 bp, Artificial Sequence
  source: /note="Description of Artificial Sequence: Syntheticoligonucleotide"
tactcaatcaatccgtgtgtagta

SEQ ID NO: 1158 - DNA, 24 bp, Artificial Sequence
  source: /note="Description of Artificial Sequence: Syntheticoligonucleotide"
gatccgaacattgtctaaccgatc

SEQ ID NO: 1159 - DNA, 24 bp, Artificial Sequence
  source: /note="Description of Artificial Sequence: Syntheticoligonucleotide"
agttagatacactgtgacaggtgg

SEQ ID NO: 1160 - DNA, 24 bp, Artificial Sequence
  source: /note="Description of Artificial Sequence: Syntheticoligonucleotide"
cgagttgtattctgcaatgagtcg

SEQ ID NO: 1161 - DNA, 24 bp, Artificial Sequence
  source: /note="Description of Artificial Sequence: Syntheticoligonucleotide"
ttgcattgctgtactcacagggac

SEQ ID NO: 1162 - DNA, 24 bp, Artificial Sequence
  source: /note="Description of Artificial Sequence: Syntheticoligonucleotide"
ggctgtaaggacggatcaaggcgc

SEQ ID NO: 1163 - DNA, 24 bp, Artificial Sequence
  source: /note="Description of Artificial Sequence: Syntheticoligonucleotide"
gatcgcatgcagcgatatccagaa

SEQ ID NO: 1164 - DNA, 24 bp, Artificial Sequence
  source: /note="Description of Artificial Sequence: Syntheticoligonucleotide"
acggcgcatatttagttgactgac

SEQ ID NO: 1165 - DNA, 24 bp, Artificial Sequence
  source: /note="Description of Artificial Sequence: Syntheticoligonucleotide"
aatgtgttctgtggaagaggatgt

SEQ ID NO: 1166 - DNA, 24 bp, Artificial Sequence
  source: /note="Description of Artificial Sequence: Syntheticoligonucleotide"
ggtgggacggaatagaagcacata

SEQ ID NO: 1167 - DNA, 24 bp, Artificial Sequence
  source: /note="Description of Artificial Sequence: Syntheticoligonucleotide"
tttaatcgacgcgcacatgtatgc

SEQ ID NO: 1168 - DNA, 24 bp, Artificial Sequence
  source: /note="Description of Artificial Sequence: Syntheticoligonucleotide"
cagggcgctggaagttactcagca

SEQ ID NO: 1169 - DNA, 24 bp, Artificial Sequence
  source: /note="Description of Artificial Sequence: Syntheticoligonucleotide"
tgtacaataaggaggcattcgaac

SEQ ID NO: 1170 - DNA, 24 bp, Artificial Sequence
  source: /note="Description of Artificial Sequence: Syntheticoligonucleotide"
cagttcccggcacgcattaagccc

SEQ ID NO: 1171 - DNA, 24 bp, Artificial Sequence
  source: /note="Description of Artificial Sequence: Syntheticoligonucleotide"
ggattcaattgtagtcgatacatg

SEQ ID NO: 1172 - DNA, 24 bp, Artificial Sequence
  source: /note="Description of Artificial Sequence: Syntheticoligonucleotide"
ctcaccattaagtagaaccggagt

SEQ ID NO: 1173 - DNA, 24 bp, Artificial Sequence
  source: /note="Description of Artificial Sequence: Syntheticoligonucleotide"
gtatagatttcgtctgcctgtatt

SEQ ID NO: 1174 - DNA, 24 bp, Artificial Sequence
  source: /note="Description of Artificial Sequence: Syntheticoligonucleotide"
ctaggagctcggccagcttgaatg

SEQ ID NO: 1175 - DNA, 24 bp, Artificial Sequence
  source: /note="Description of Artificial Sequence: Syntheticoligonucleotide"
cagaaagctgcgaatagtttgatg

SEQ ID NO: 1176 - DNA, 24 bp, Artificial Sequence
  source: /note="Description of Artificial Sequence: Syntheticoligonucleotide"
tgggtattctatcgcgcgtaagca

SEQ ID NO: 1177 - DNA, 24 bp, Artificial Sequence
  source: /note="Description of Artificial Sequence: Syntheticoligonucleotide"
gacccagaattacgcctccaacct

SEQ ID NO: 1178 - DNA, 24 bp, Artificial Sequence
  source: /note="Description of Artificial Sequence: Syntheticoligonucleotide"
tgtactccggccaccctaaacttt

SEQ ID NO: 1179 - DNA, 24 bp, Artificial Sequence
  source: /note="Description of Artificial Sequence: Syntheticoligonucleotide"
attggcgacgggactatttcttaa

SEQ ID NO: 1180 - DNA, 24 bp, Artificial Sequence
  source: /note="Description of Artificial Sequence: Syntheticoligonucleotide"
tctaggcccggagagatagtatca

SEQ ID NO: 1181 - DNA, 24 bp, Artificial Sequence
  source: /note="Description of Artificial Sequence: Syntheticoligonucleotide"
tcatgcatagtttccttactagtt

SEQ ID NO: 1182 - DNA, 24 bp, Artificial Sequence
  source: /note="Description of Artificial Sequence: Syntheticoligonucleotide"
aggtctaggagacagaaacgtatt

SEQ ID NO: 1183 - DNA, 24 bp, Artificial Sequence
  source: /note="Description of Artificial Sequence: Syntheticoligonucleotide"
actcagaaaggcgaatcgatgacc

SEQ ID NO: 1184 - DNA, 24 bp, Artificial Sequence
  source: /note="Description of Artificial Sequence: Syntheticoligonucleotide"
tcggtaagttaaccatatgcctca

SEQ ID NO: 1185 - DNA, 24 bp, Artificial Sequence
  source: /note="Description of Artificial Sequence: Syntheticoligonucleotide"
acgtgataaacatccatccatacg

SEQ ID NO: 1186 - DNA, 24 bp, Artificial Sequence
  source: /note="Description of Artificial Sequence: Syntheticoligonucleotide"
ctaatttatcgaaacggccaacac

SEQ ID NO: 1187 - DNA, 24 bp, Artificial Sequence
  source: /note="Description of Artificial Sequence: Syntheticoligonucleotide"
aagcgtgtttcatgacgagcaggg

SEQ ID NO: 1188 - DNA, 24 bp, Artificial Sequence
  source: /note="Description of Artificial Sequence: Syntheticoligonucleotide"
ttaatgctgatttcgagagcgccg

SEQ ID NO: 1189 - DNA, 24 bp, Artificial Sequence
  source: /note="Description of Artificial Sequence: Syntheticoligonucleotide"
aaagtcgagagtaacccgtaacgt

SEQ ID NO: 1190 - DNA, 24 bp, Artificial Sequence
  source: /note="Description of Artificial Sequence: Syntheticoligonucleotide"
tctgagataaggactcctggactg

SEQ ID NO: 1191 - DNA, 24 bp, Artificial Sequence
  source: /note="Description of Artificial Sequence: Syntheticoligonucleotide"
tcaggttagtgaaaggaagtgagg

SEQ ID NO: 1192 - DNA, 24 bp, Artificial Sequence
  source: /note="Description of Artificial Sequence: Syntheticoligonucleotide"
tggcgttgctgagcgaaagagtaa

SEQ ID NO: 1193 - DNA, 24 bp, Artificial Sequence
  source: /note="Description of Artificial Sequence: Syntheticoligonucleotide"
tagggcctgccgatacagttcgcc

SEQ ID NO: 1194 - DNA, 24 bp, Artificial Sequence
  source: /note="Description of Artificial Sequence: Syntheticoligonucleotide"
gtacacaactagcctctggcgact

SEQ ID NO: 1195 - DNA, 24 bp, Artificial Sequence
  source: /note="Description of Artificial Sequence: Syntheticoligonucleotide"
catatagtaggctgcacagataga

SEQ ID NO: 1196 - DNA, 24 bp, Artificial Sequence
  source: /note="Description of Artificial Sequence: Syntheticoligonucleotide"
agttgacgtcgctcgcttaaggaa

SEQ ID NO: 1197 - DNA, 24 bp, Artificial Sequence
  source: /note="Description of Artificial Sequence: Syntheticoligonucleotide"
aacatatccagggaggtgatccat

SEQ ID NO: 1198 - DNA, 24 bp, Artificial Sequence
  source: /note="Description of Artificial Sequence: Syntheticoligonucleotide"
atgggctaagaatagtttgagatg

SEQ ID NO: 1199 - DNA, 24 bp, Artificial Sequence
  source: /note="Description of Artificial Sequence: Syntheticoligonucleotide"
cagatcttggtagtagttggacag

SEQ ID NO: 1200 - DNA, 24 bp, Artificial Sequence
  source: /note="Description of Artificial Sequence: Syntheticoligonucleotide"
tagtcccagttacggtaccttcca

SEQ ID NO: 1201 - DNA, 24 bp, Artificial Sequence
  source: /note="Description of Artificial Sequence: Syntheticoligonucleotide"
ggtacacttgcgtatccgacgctt

SEQ ID NO: 1202 - DNA, 24 bp, Artificial Sequence
  source: /note="Description of Artificial Sequence: Syntheticoligonucleotide"
aagtggacaacggcgacatatata

SEQ ID NO: 1203 - DNA, 24 bp, Artificial Sequence
  source: /note="Description of Artificial Sequence: Syntheticoligonucleotide"
gcgggctcataacgaatccggctg

SEQ ID NO: 1204 - DNA, 24 bp, Artificial Sequence
  source: /note="Description of Artificial Sequence: Syntheticoligonucleotide"
ccgcaacacagcccatagcagcta

SEQ ID NO: 1205 - DNA, 24 bp, Artificial Sequence
  source: /note="Description of Artificial Sequence: Syntheticoligonucleotide"
acctgcttatttgtaacgatcggg

SEQ ID NO: 1206 - DNA, 24 bp, Artificial Sequence
  source: /note="Description of Artificial Sequence: Syntheticoligonucleotide"
aacttcgatgtctgtacacgtact

SEQ ID NO: 1207 - DNA, 24 bp, Artificial Sequence
  source: /note="Description of Artificial Sequence: Syntheticoligonucleotide"
atgaatgacagacctacctgtcca

SEQ ID NO: 1208 - DNA, 24 bp, Artificial Sequence
  source: /note="Description of Artificial Sequence: Syntheticoligonucleotide"
ctactgtggttagaccgatctgag

SEQ ID NO: 1209 - DNA, 24 bp, Artificial Sequence
  source: /note="Description of Artificial Sequence: Syntheticoligonucleotide"
gaacctcggaactagatcttactt

SEQ ID NO: 1210 - DNA, 24 bp, Artificial Sequence
  source: /note="Description of Artificial Sequence: Syntheticoligonucleotide"
tggaatcttgaaaggtcactcacc

SEQ ID NO: 1211 - DNA, 24 bp, Artificial Sequence
  source: /note="Description of Artificial Sequence: Syntheticoligonucleotide"
caagaccaagcagtcagcggattt

SEQ ID NO: 1212 - DNA, 24 bp, Artificial Sequence
  source: /note="Description of Artificial Sequence: Syntheticoligonucleotide"
gatgtaggctcctcgttctatgtt

SEQ ID NO: 1213 - DNA, 24 bp, Artificial Sequence
  source: /note="Description of Artificial Sequence: Syntheticoligonucleotide"
cattactcctgtgttagctagtca

SEQ ID NO: 1214 - DNA, 24 bp, Artificial Sequence
  source: /note="Description of Artificial Sequence: Syntheticoligonucleotide"
gttcaatcgtgacttccgatgtgt

SEQ ID NO: 1215 - DNA, 24 bp, Artificial Sequence
  source: /note="Description of Artificial Sequence: Syntheticoligonucleotide"
tggctggaacactcaggatttgta

SEQ ID NO: 1216 - DNA, 24 bp, Artificial Sequence
  source: /note="Description of Artificial Sequence: Syntheticoligonucleotide"
ctcttaccactaggcacactaata

SEQ ID NO: 1217 - DNA, 24 bp, Artificial Sequence
  source: /note="Description of Artificial Sequence: Syntheticoligonucleotide"
caattagcatacgccgtacttgtt

SEQ ID NO: 1218 - DNA, 24 bp, Artificial Sequence
  source: /note="Description of Artificial Sequence: Syntheticoligonucleotide"
ttgagttagagcactgtcgtatgt

SEQ ID NO: 1219 - DNA, 24 bp, Artificial Sequence
  source: /note="Description of Artificial Sequence: Syntheticoligonucleotide"
accagtttggaagtgtttgttgag

SEQ ID NO: 1220 - DNA, 24 bp, Artificial Sequence
  source: /note="Description of Artificial Sequence: Syntheticoligonucleotide"
agctgcttgaatgtgaaccagcat

SEQ ID NO: 1221 - DNA, 24 bp, Artificial Sequence
  source: /note="Description of Artificial Sequence: Syntheticoligonucleotide"
atgtacaggagaatggtgggaagt

SEQ ID NO: 1222 - DNA, 24 bp, Artificial Sequence
  source: /note="Description of Artificial Sequence: Syntheticoligonucleotide"
cgaaacctacgagttatatgtgat

SEQ ID NO: 1223 - DNA, 24 bp, Artificial Sequence
  source: /note="Description of Artificial Sequence: Syntheticoligonucleotide"
gtctctttgctccaagtcctagag

SEQ ID NO: 1224 - DNA, 24 bp, Artificial Sequence
  source: /note="Description of Artificial Sequence: Syntheticoligonucleotide"
taactacaccgtgggtttgcgttc

SEQ ID NO: 1225 - DNA, 24 bp, Artificial Sequence
  source: /note="Description of Artificial Sequence: Syntheticoligonucleotide"
gaaagaatccaacgtagttaaatc

SEQ ID NO: 1226 - DNA, 24 bp, Artificial Sequence
  source: /note="Description of Artificial Sequence: Syntheticoligonucleotide"
ctacgtgcagtaatcagatgatat

SEQ ID NO: 1227 - DNA, 24 bp, Artificial Sequence
  source: /note="Description of Artificial Sequence: Syntheticoligonucleotide"
ctaagccctttgtctgacacgaag

SEQ ID NO: 1228 - DNA, 24 bp, Artificial Sequence
  source: /note="Description of Artificial Sequence: Syntheticoligonucleotide"
atcattgcattgtgtcaaattaat

SEQ ID NO: 1229 - DNA, 24 bp, Artificial Sequence
  source: /note="Description of Artificial Sequence: Syntheticoligonucleotide"
tgttggcgtcaattctattgtgat

SEQ ID NO: 1230 - DNA, 24 bp, Artificial Sequence
  source: /note="Description of Artificial Sequence: Syntheticoligonucleotide"
gtggctgtactctatattgtatct

SEQ ID NO: 1231 - DNA, 24 bp, Artificial Sequence
  source: /note="Description of Artificial Sequence: Syntheticoligonucleotide"
aagcattgttgttgccgcaaagca

SEQ ID NO: 1232 - DNA, 24 bp, Artificial Sequence
  source: /note="Description of Artificial Sequence: Syntheticoligonucleotide"
cactgacccaaaccttaatacgaa

SEQ ID NO: 1233 - DNA, 24 bp, Artificial Sequence
  source: /note="Description of Artificial Sequence: Syntheticoligonucleotide"
ccacgcacatcgtactcttaatag

SEQ ID NO: 1234 - DNA, 24 bp, Artificial Sequence
  source: /note="Description of Artificial Sequence: Syntheticoligonucleotide"
tgcattgcttgatagttacgccaa

SEQ ID NO: 1235 - DNA, 24 bp, Artificial Sequence
  source: /note="Description of Artificial Sequence: Syntheticoligonucleotide"
tagggtcttgcctcggaccgggtt

SEQ ID NO: 1236 - DNA, 24 bp, Artificial Sequence
  source: /note="Description of Artificial Sequence: Syntheticoligonucleotide"
gtagaacgtgattcagtccttcgt

SEQ ID NO: 1237 - DNA, 24 bp, Artificial Sequence
  source: /note="Description of Artificial Sequence: Syntheticoligonucleotide"
cactaaggtgaaatatcctgtgcc

SEQ ID NO: 1238 - DNA, 24 bp, Artificial Sequence
  source: /note="Description of Artificial Sequence: Syntheticoligonucleotide"
cacttgtcgtcctcttggttatgc

SEQ ID NO: 1239 - DNA, 24 bp, Artificial Sequence
  source: /note="Description of Artificial Sequence: Syntheticoligonucleotide"
tcccaaacacttagatcctcgtgt

SEQ ID NO: 1240 - DNA, 24 bp, Artificial Sequence
  source: /note="Description of Artificial Sequence: Syntheticoligonucleotide"
ccctcgttatttgtattggtaggt

SEQ ID NO: 1241 - DNA, 24 bp, Artificial Sequence
  source: /note="Description of Artificial Sequence: Syntheticoligonucleotide"
aaggtaaacctagtaaagcctggc

SEQ ID NO: 1242 - DNA, 24 bp, Artificial Sequence
  source: /note="Description of Artificial Sequence: Syntheticoligonucleotide"
accgggtatgagcaaatgacccac

SEQ ID NO: 1243 - DNA, 24 bp, Artificial Sequence
  source: /note="Description of Artificial Sequence: Syntheticoligonucleotide"
aggtggtgtctcatgctgctaaac

SEQ ID NO: 1244 - DNA, 24 bp, Artificial Sequence
  source: /note="Description of Artificial Sequence: Syntheticoligonucleotide"
aatataagcgagtcatgctaactg

SEQ ID NO: 1245 - DNA, 24 bp, Artificial Sequence
  source: /note="Description of Artificial Sequence: Syntheticoligonucleotide"
ctggcaaatctacaaagtcttgtc

SEQ ID NO: 1246 - DNA, 24 bp, Artificial Sequence
  source: /note="Description of Artificial Sequence: Syntheticoligonucleotide"
ctatgcagcatttggatctatcag

SEQ ID NO: 1247 - DNA, 24 bp, Artificial Sequence
  source: /note="Description of Artificial Sequence: Syntheticoligonucleotide"
ataaacagtgcaggacatactggc

SEQ ID NO: 1248 - DNA, 24 bp, Artificial Sequence
  source: /note="Description of Artificial Sequence: Syntheticoligonucleotide"
gtggacaggaacaccaaacggatt

SEQ ID NO: 1249 - DNA, 24 bp, Artificial Sequence
  source: /note="Description of Artificial Sequence: Syntheticoligonucleotide"
gtctcggccatctacataccgtat

SEQ ID NO: 1250 - DNA, 24 bp, Artificial Sequence
  source: /note="Description of Artificial Sequence: Syntheticoligonucleotide"
atccctgccacacatcggctcgat

SEQ ID NO: 1251 - DNA, 24 bp, Artificial Sequence
  source: /note="Description of Artificial Sequence: Syntheticoligonucleotide"
actaaacctcctgctctgcgcgta

SEQ ID NO: 1252 - DNA, 24 bp, Artificial Sequence
  source: /note="Description of Artificial Sequence: Syntheticoligonucleotide"
cgataagcgtcaaaggtgccttga

SEQ ID NO: 1253 - DNA, 24 bp, Artificial Sequence
  source: /note="Description of Artificial Sequence: Syntheticoligonucleotide"
acacgtcttatcgccgtagcagaa

SEQ ID NO: 1254 - DNA, 24 bp, Artificial Sequence
  source: /note="Description of Artificial Sequence: Syntheticoligonucleotide"
atgcccgagcagaattgcccttaa

SEQ ID NO: 1255 - DNA, 24 bp, Artificial Sequence
  source: /note="Description of Artificial Sequence: Syntheticoligonucleotide"
gactgtctgcaataacagatgtgt

SEQ ID NO: 1256 - DNA, 24 bp, Artificial Sequence
  source: /note="Description of Artificial Sequence: Syntheticoligonucleotide"
cgggtagatgtgaatcgatatctt

SEQ ID NO: 1257 - DNA, 24 bp, Artificial Sequence
  source: /note="Description of Artificial Sequence: Syntheticoligonucleotide"
ttggcgtgtatcatttatcgagtt

SEQ ID NO: 1258 - DNA, 24 bp, Artificial Sequence
  source: /note="Description of Artificial Sequence: Syntheticoligonucleotide"
cactcgtcgtcgattatggacatt

SEQ ID NO: 1259 - DNA, 24 bp, Artificial Sequence
  source: /note="Description of Artificial Sequence: Syntheticoligonucleotide"
atgcatatggaccctacttgtctg

SEQ ID NO: 1260 - DNA, 24 bp, Artificial Sequence
  source: /note="Description of Artificial Sequence: Syntheticoligonucleotide"
aaagatactcaaacttgttgcagc

SEQ ID NO: 1261 - DNA, 24 bp, Artificial Sequence
  source: /note="Description of Artificial Sequence: Syntheticoligonucleotide"
ttgacagacattcgtgggttccta

SEQ ID NO: 1262 - DNA, 24 bp, Artificial Sequence
  source: /note="Description of Artificial Sequence: Syntheticoligonucleotide"
cctttcacagagccggcatgaaag

SEQ ID NO: 1263 - DNA, 24 bp, Artificial Sequence
  source: /note="Description of Artificial Sequence: Syntheticoligonucleotide"
tgggtgtttatatcccgacagaac

SEQ ID NO: 1264 - DNA, 24 bp, Artificial Sequence
  source: /note="Description of Artificial Sequence: Syntheticoligonucleotide"
cgctttggtcagaaatgaaaccgg

SEQ ID NO: 1265 - DNA, 24 bp, Artificial Sequence
  source: /note="Description of Artificial Sequence: Syntheticoligonucleotide"
ctaggtggtaaagaacttgacgtt

SEQ ID NO: 1266 - DNA, 24 bp, Artificial Sequence
  source: /note="Description of Artificial Sequence: Syntheticoligonucleotide"
attgtgacccgcagtatcctaacg

SEQ ID NO: 1267 - DNA, 24 bp, Artificial Sequence
  source: /note="Description of Artificial Sequence: Syntheticoligonucleotide"
cgaagttggacctgacacgacaat

SEQ ID NO: 1268 - DNA, 24 bp, Artificial Sequence
  source: /note="Description of Artificial Sequence: Syntheticoligonucleotide"
aacgctaatgatgtttctcatcgg

SEQ ID NO: 1269 - DNA, 24 bp, Artificial Sequence
  source: /note="Description of Artificial Sequence: Syntheticoligonucleotide"
gcctcaaacggtgacgaaggatac

SEQ ID NO: 1270 - DNA, 24 bp, Artificial Sequence
  source: /note="Description of Artificial Sequence: Syntheticoligonucleotide"
acctgcgtacctctcacccatagg

SEQ ID NO: 1271 - DNA, 24 bp, Artificial Sequence
  source: /note="Description of Artificial Sequence: Syntheticoligonucleotide"
tctacgttatcgcaaccgacttac

SEQ ID NO: 1272 - DNA, 24 bp, Artificial Sequence
  source: /note="Description of Artificial Sequence: Syntheticoligonucleotide"
atgtaataggctcacccgctccaa

SEQ ID NO: 1273 - DNA, 24 bp, Artificial Sequence
  source: /note="Description of Artificial Sequence: Syntheticoligonucleotide"
actaacacgttgggcattggaaat

SEQ ID NO: 1274 - DNA, 24 bp, Artificial Sequence
  source: /note="Description of Artificial Sequence: Syntheticoligonucleotide"
tgtattcttctcgtacgtgagaac

SEQ ID NO: 1275 - DNA, 24 bp, Artificial Sequence
  source: /note="Description of Artificial Sequence: Syntheticoligonucleotide"
actactcttgtatatcgggtgtcc

SEQ ID NO: 1276 - DNA, 24 bp, Artificial Sequence
  source: /note="Description of Artificial Sequence: Syntheticoligonucleotide"
cacgatgcggaagtgaattcgata

SEQ ID NO: 1277 - DNA, 24 bp, Artificial Sequence
  source: /note="Description of Artificial Sequence: Syntheticoligonucleotide"
ctctggtaaacattagtgtagcgg

SEQ ID NO: 1278 - DNA, 24 bp, Artificial Sequence
  source: /note="Description of Artificial Sequence: Syntheticoligonucleotide"
ttattctaagtcgacctcatctcg

SEQ ID NO: 1279 - DNA, 24 bp, Artificial Sequence
  source: /note="Description of Artificial Sequence: Syntheticoligonucleotide"
attacccagtaatctaagcttcct

SEQ ID NO: 1280 - DNA, 24 bp, Artificial Sequence
  source: /note="Description of Artificial Sequence: Syntheticoligonucleotide"
tacgaaacaggtgataattgcagc

SEQ ID NO: 1281 - DNA, 24 bp, Artificial Sequence
  source: /note="Description of Artificial Sequence: Syntheticoligonucleotide"
ctattgactaaacgaaagccctca

SEQ ID NO: 1282 - DNA, 24 bp, Artificial Sequence
  source: /note="Description of Artificial Sequence: Syntheticoligonucleotide"
tgagatagcccaacccatcaagaa

SEQ ID NO: 1283 - DNA, 24 bp, Artificial Sequence
  source: /note="Description of Artificial Sequence: Syntheticoligonucleotide"
taccgacatcctgtatcaacattc

SEQ ID NO: 1284 - DNA, 24 bp, Artificial Sequence
  source: /note="Description of Artificial Sequence: Syntheticoligonucleotide"
tccatgcgattgtcctgtttaggt

SEQ ID NO: 1285 - DNA, 24 bp, Artificial Sequence
  source: /note="Description of Artificial Sequence: Syntheticoligonucleotide"
gtatagacaattcagtaaggaggc

SEQ ID NO: 1286 - DNA, 24 bp, Artificial Sequence
  source: /note="Description of Artificial Sequence: Syntheticoligonucleotide"
ggagcctcatcaacctaggactac

SEQ ID NO: 1287 - DNA, 24 bp, Artificial Sequence
  source: /note="Description of Artificial Sequence: Syntheticoligonucleotide"
atttgtaacgcccttcgaattgat

SEQ ID NO: 1288 - DNA, 24 bp, Artificial Sequence
  source: /note="Description of Artificial Sequence: Syntheticoligonucleotide"
ccaattagggattatctagagtga

SEQ ID NO: 1289 - DNA, 24 bp, Artificial Sequence
  source: /note="Description of Artificial Sequence: Syntheticoligonucleotide"
gtttaacgaccgcaaccattagac

SEQ ID NO: 1290 - DNA, 24 bp, Artificial Sequence
  source: /note="Description of Artificial Sequence: Syntheticoligonucleotide"
gccgacagttatacacttaaggcg

SEQ ID NO: 1291 - DNA, 24 bp, Artificial Sequence
  source: /note="Description of Artificial Sequence: Syntheticoligonucleotide"
acgtcagccacgagagaatgagat

SEQ ID NO: 1292 - DNA, 24 bp, Artificial Sequence
  source: /note="Description of Artificial Sequence: Syntheticoligonucleotide"
aagggtaactaggcggctcgtaat

SEQ ID NO: 1293 - DNA, 24 bp, Artificial Sequence
  source: /note="Description of Artificial Sequence: Syntheticoligonucleotide"
agccatcgtgtcgtctggaatcca

SEQ ID NO: 1294 - DNA, 24 bp, Artificial Sequence
  source: /note="Description of Artificial Sequence: Syntheticoligonucleotide"
tagtagcgcgaaagtttgcccaat

SEQ ID NO: 1295 - DNA, 24 bp, Artificial Sequence
  source: /note="Description of Artificial Sequence: Syntheticoligonucleotide"
agaacagaccctcgatcgtttaag

SEQ ID NO: 1296 - DNA, 24 bp, Artificial Sequence
  source: /note="Description of Artificial Sequence: Syntheticoligonucleotide"
atttcacaaccataccgggagatt

SEQ ID NO: 1297 - DNA, 24 bp, Artificial Sequence
  source: /note="Description of Artificial Sequence: Syntheticoligonucleotide"
tacctaggcacggcttagtctagc

SEQ ID NO: 1298 - DNA, 24 bp, Artificial Sequence
  source: /note="Description of Artificial Sequence: Syntheticoligonucleotide"
ctcatagccgtgaaggtgtaggac

SEQ ID NO: 1299 - DNA, 24 bp, Artificial Sequence
  source: /note="Description of Artificial Sequence: Syntheticoligonucleotide"
atcgaacctagagcacgttaatcc

SEQ ID NO: 1300 - DNA, 24 bp, Artificial Sequence
  source: /note="Description of Artificial Sequence: Syntheticoligonucleotide"
ggcagcgagtgtaaaggacctctg

SEQ ID NO: 1301 - DNA, 24 bp, Artificial Sequence
  source: /note="Description of Artificial Sequence: Syntheticoligonucleotide"
gctttaatccgcctacgatgccct

SEQ ID NO: 1302 - DNA, 24 bp, Artificial Sequence
  source: /note="Description of Artificial Sequence: Syntheticoligonucleotide"
actcacacaattactacagagcta

SEQ ID NO: 1303 - DNA, 24 bp, Artificial Sequence
  source: /note="Description of Artificial Sequence: Syntheticoligonucleotide"
tgaccattgggtctgtactagcca

SEQ ID NO: 1304 - DNA, 24 bp, Artificial Sequence
  source: /note="Description of Artificial Sequence: Syntheticoligonucleotide"
gtaaaccagtaaccaagttctgct

SEQ ID NO: 1305 - DNA, 24 bp, Artificial Sequence
  source: /note="Description of Artificial Sequence: Syntheticoligonucleotide"
gattatcgaggacacgggagcata

SEQ ID NO: 1306 - DNA, 24 bp, Artificial Sequence
  source: /note="Description of Artificial Sequence: Syntheticoligonucleotide"
caccgttgtctttagtccaccact

SEQ ID NO: 1307 - DNA, 24 bp, Artificial Sequence
  source: /note="Description of Artificial Sequence: Syntheticoligonucleotide"
atctacggcgacgtccataccttt

SEQ ID NO: 1308 - DNA, 24 bp, Artificial Sequence
  source: /note="Description of Artificial Sequence: Syntheticoligonucleotide"
tgagggacacccactcgaattacc

SEQ ID NO: 1309 - DNA, 24 bp, Artificial Sequence
  source: /note="Description of Artificial Sequence: Syntheticoligonucleotide"
gaccaggcaacaccaggaagattg

SEQ ID NO: 1310 - DNA, 24 bp, Artificial Sequence
  source: /note="Description of Artificial Sequence: Syntheticoligonucleotide"
ttaggcagctgatcggactcttaa

SEQ ID NO: 1311 - DNA, 24 bp, Artificial Sequence
  source: /note="Description of Artificial Sequence: Syntheticoligonucleotide"
tctcctgacgatttccaaacatcc

SEQ ID NO: 1312 - DNA, 24 bp, Artificial Sequence
  source: /note="Description of Artificial Sequence: Syntheticoligonucleotide"
gcatggtaacttagacctctggaa

SEQ ID NO: 1313 - DNA, 24 bp, Artificial Sequence
  source: /note="Description of Artificial Sequence: Syntheticoligonucleotide"
taaggagtgtctcttgtctgtcaa

SEQ ID NO: 1314 - DNA, 24 bp, Artificial Sequence
  source: /note="Description of Artificial Sequence: Syntheticoligonucleotide"
gtaatttgctgttaccaatgcact

SEQ ID NO: 1315 - DNA, 24 bp, Artificial Sequence
  source: /note="Description of Artificial Sequence: Syntheticoligonucleotide"
agccctatcgtagtagaccgtatc

SEQ ID NO: 1316 - DNA, 24 bp, Artificial Sequence
  source: /note="Description of Artificial Sequence: Syntheticoligonucleotide"
cttatccatcaacactgcatccct

SEQ ID NO: 1317 - DNA, 24 bp, Artificial Sequence
  source: /note="Description of Artificial Sequence: Syntheticoligonucleotide"
gggcgaaccaatcgatctatcgtg

SEQ ID NO: 1318 - DNA, 24 bp, Artificial Sequence
  source: /note="Description of Artificial Sequence: Syntheticoligonucleotide"
gtgataccgtcattgggaatcata

SEQ ID NO: 1319 - DNA, 24 bp, Artificial Sequence
  source: /note="Description of Artificial Sequence: Syntheticoligonucleotide"
gaaccacagatctttctttgtgga

SEQ ID NO: 1320 - DNA, 24 bp, Artificial Sequence
  source: /note="Description of Artificial Sequence: Syntheticoligonucleotide"
ctttcgtttgcaacgaggcagagc

SEQ ID NO: 1321 - DNA, 24 bp, Artificial Sequence
  source: /note="Description of Artificial Sequence: Syntheticoligonucleotide"
tttgtgaaatacattctaaggcag

SEQ ID NO: 1322 - DNA, 24 bp, Artificial Sequence
  source: /note="Description of Artificial Sequence: Syntheticoligonucleotide"
atgggctgaatttagtgccatgac

SEQ ID NO: 1323 - DNA, 24 bp, Artificial Sequence
  source: /note="Description of Artificial Sequence: Syntheticoligonucleotide"
ggtttaggccatattgcatctctg

SEQ ID NO: 1324 - DNA, 24 bp, Artificial Sequence
  source: /note="Description of Artificial Sequence: Syntheticoligonucleotide"
cgaaagggcgtcagagtttctttc

SEQ ID NO: 1325 - DNA, 24 bp, Artificial Sequence
  source: /note="Description of Artificial Sequence: Syntheticoligonucleotide"
gtgactcggttcggcacattcatg

SEQ ID NO: 1326 - DNA, 24 bp, Artificial Sequence
  source: /note="Description of Artificial Sequence: Syntheticoligonucleotide"
caataaatggatgtcttgtggctc

SEQ ID NO: 1327 - DNA, 24 bp, Artificial Sequence
  source: /note="Description of Artificial Sequence: Syntheticoligonucleotide"
ccggtccaagcgaaccctacaatg

SEQ ID NO: 1328 - DNA, 24 bp, Artificial Sequence
  source: /note="Description of Artificial Sequence: Syntheticoligonucleotide"
aagcgctgacaacgtcgtgatggg

SEQ ID NO: 1329 - DNA, 24 bp, Artificial Sequence
  source: /note="Description of Artificial Sequence: Syntheticoligonucleotide"
atacacacactatttactgactga

SEQ ID NO: 1330 - DNA, 24 bp, Artificial Sequence
  source: /note="Description of Artificial Sequence: Syntheticoligonucleotide"
gcttcccactactcgtagaaactt

SEQ ID NO: 1331 - DNA, 24 bp, Artificial Sequence
  source: /note="Description of Artificial Sequence: Syntheticoligonucleotide"
tccctgtccgcgtgagctctgata

SEQ ID NO: 1332 - DNA, 24 bp, Artificial Sequence
  source: /note="Description of Artificial Sequence: Syntheticoligonucleotide"
cattatgtccatctagcgcatcct

SEQ ID NO: 1333 - DNA, 24 bp, Artificial Sequence
  source: /note="Description of Artificial Sequence: Syntheticoligonucleotide"
acactgcctagtcgaaggcgtctt

SEQ ID NO: 1334 - DNA, 24 bp, Artificial Sequence
  source: /note="Description of Artificial Sequence: Syntheticoligonucleotide"
ccaacataagttcgccgccttggt

SEQ ID NO: 1335 - DNA, 24 bp, Artificial Sequence
  source: /note="Description of Artificial Sequence: Syntheticoligonucleotide"
tcgcatgacgagcatttgcgagtg

SEQ ID NO: 1336 - DNA, 24 bp, Artificial Sequence
  source: /note="Description of Artificial Sequence: Syntheticoligonucleotide"
accgcaaggttgttattgtgggac

SEQ ID NO: 1337 - DNA, 24 bp, Artificial Sequence
  source: /note="Description of Artificial Sequence: Syntheticoligonucleotide"
gctacaactttcaagtaaactcgc

SEQ ID NO: 1338 - DNA, 24 bp, Artificial Sequence
  source: /note="Description of Artificial Sequence: Syntheticoligonucleotide"
gattcttcgtagacgggttatggt

SEQ ID NO: 1339 - DNA, 24 bp, Artificial Sequence
  source: /note="Description of Artificial Sequence: Syntheticoligonucleotide"
gcatttaaagcacgtaccgttcta

SEQ ID NO: 1340 - DNA, 24 bp, Artificial Sequence
  source: /note="Description of Artificial Sequence: Syntheticoligonucleotide"
cgcccaaggacaaattcatcatat

SEQ ID NO: 1341 - DNA, 24 bp, Artificial Sequence
  source: /note="Description of Artificial Sequence: Syntheticoligonucleotide"
cgtaaagacttacatccctagatg

SEQ ID NO: 1342 - DNA, 24 bp, Artificial Sequence
  source: /note="Description of Artificial Sequence: Syntheticoligonucleotide"
taatataagcgcgtgaatcgtgtg

SEQ ID NO: 1343 - DNA, 24 bp, Artificial Sequence
  source: /note="Description of Artificial Sequence: Syntheticoligonucleotide"
attgattgagtaatagacgaacgc

SEQ ID NO: 1344 - DNA, 24 bp, Artificial Sequence
  source: /note="Description of Artificial Sequence: Syntheticoligonucleotide"
aatgttcggatcctcgcactatga

SEQ ID NO: 1345 - DNA, 24 bp, Artificial Sequence
  source: /note="Description of Artificial Sequence: Syntheticoligonucleotide"
gtgtatctaactgcttaacatacc

SEQ ID NO: 1346 - DNA, 24 bp, Artificial Sequence
  source: /note="Description of Artificial Sequence: Syntheticoligonucleotide"
gaatacaactcgttgggcagtaca

SEQ ID NO: 1347 - DNA, 24 bp, Artificial Sequence
  source: /note="Description of Artificial Sequence: Syntheticoligonucleotide"
acagcaatgcaaataattggcggc

SEQ ID NO: 1348 - DNA, 24 bp, Artificial Sequence
  source: /note="Description of Artificial Sequence: Syntheticoligonucleotide"
gtccttacagcctttatgaacttt

SEQ ID NO: 1349 - DNA, 24 bp, Artificial Sequence
  source: /note="Description of Artificial Sequence: Syntheticoligonucleotide"
ctgcatgcgatcagatcaatgctt

SEQ ID NO: 1350 - DNA, 24 bp, Artificial Sequence
  source: /note="Description of Artificial Sequence: Syntheticoligonucleotide"
aatatgcgccgtggcttaaggtga

SEQ ID NO: 1351 - DNA, 24 bp, Artificial Sequence
  source: /note="Description of Artificial Sequence: Syntheticoligonucleotide"
acagaacacattcctactccgttt

SEQ ID NO: 1352 - DNA, 24 bp, Artificial Sequence
  source: /note="Description of Artificial Sequence: Syntheticoligonucleotide"
ttccgtcccacccaacaactatag

SEQ ID NO: 1353 - DNA, 24 bp, Artificial Sequence
  source: /note="Description of Artificial Sequence: Syntheticoligonucleotide"
gcgtcgattaaattgatatagacc

SEQ ID NO: 1354 - DNA, 24 bp, Artificial Sequence
  source: /note="Description of Artificial Sequence: Syntheticoligonucleotide"
tccagcgccctgcccattataagt

SEQ ID NO: 1355 - DNA, 24 bp, Artificial Sequence
  source: /note="Description of Artificial Sequence: Syntheticoligonucleotide"
ccttattgtacagggttaactact

SEQ ID NO: 1356 - DNA, 24 bp, Artificial Sequence
  source: /note="Description of Artificial Sequence: Syntheticoligonucleotide"
tgccgggaactgaaacaagatatt

SEQ ID NO: 1357 - DNA, 24 bp, Artificial Sequence
  source: /note="Description of Artificial Sequence: Syntheticoligonucleotide"
acaattgaatcccggatgactccg

SEQ ID NO: 1358 - DNA, 24 bp, Artificial Sequence
  source: /note="Description of Artificial Sequence: Syntheticoligonucleotide"
cttaatggtgaggtaaccggacga

SEQ ID NO: 1359 - DNA, 24 bp, Artificial Sequence
  source: /note="Description of Artificial Sequence: Syntheticoligonucleotide"
cgaaatctatactcggctgtgaat

SEQ ID NO: 1360 - DNA, 24 bp, Artificial Sequence
  source: /note="Description of Artificial Sequence: Syntheticoligonucleotide"
ccgagctcctagtagtatagttac

SEQ ID NO: 1361 - DNA, 24 bp, Artificial Sequence
  source: /note="Description of Artificial Sequence: Syntheticoligonucleotide"
cgcagctttctgcggtcatgaagg

SEQ ID NO: 1362 - DNA, 24 bp, Artificial Sequence
  source: /note="Description of Artificial Sequence: Syntheticoligonucleotide"
atagaatacccaaagagcatataa

SEQ ID NO: 1363 - DNA, 24 bp, Artificial Sequence
  source: /note="Description of Artificial Sequence: Syntheticoligonucleotide"
taattctgggtccatcatcgacaa

SEQ ID NO: 1364 - DNA, 24 bp, Artificial Sequence
  source: /note="Description of Artificial Sequence: Syntheticoligonucleotide"
ggccggagtacaatctagtcaccc

SEQ ID NO: 1365 - DNA, 24 bp, Artificial Sequence
  source: /note="Description of Artificial Sequence: Syntheticoligonucleotide"
cccgtcgccaatcatgctccagcc

SEQ ID NO: 1366 - DNA, 24 bp, Artificial Sequence
  source: /note="Description of Artificial Sequence: Syntheticoligonucleotide"
tccgggcctagaaacagtgcttgc

SEQ ID NO: 1367 - DNA, 24 bp, Artificial Sequence
  source: /note="Description of Artificial Sequence: Syntheticoligonucleotide"
aactatgcatgagacttgctgctg

SEQ ID NO: 1368 - DNA, 24 bp, Artificial Sequence
  source: /note="Description of Artificial Sequence: Syntheticoligonucleotide"
tctcctagacctccggcacgacaa

SEQ ID NO: 1369 - DNA, 24 bp, Artificial Sequence
  source: /note="Description of Artificial Sequence: Syntheticoligonucleotide"
gcctttctgagtcgcaaagtcatt

SEQ ID NO: 1370 - DNA, 24 bp, Artificial Sequence
  source: /note="Description of Artificial Sequence: Syntheticoligonucleotide"
ttaacttaccgagcaagcttctta

SEQ ID NO: 1371 - DNA, 24 bp, Artificial Sequence
  source: /note="Description of Artificial Sequence: Syntheticoligonucleotide"
tgtttatcacgtgcgcgagagatt

SEQ ID NO: 1372 - DNA, 24 bp, Artificial Sequence
  source: /note="Description of Artificial Sequence: Syntheticoligonucleotide"
tcgataaattagagttctggtcta

SEQ ID NO: 1373 - DNA, 24 bp, Artificial Sequence
  source: /note="Description of Artificial Sequence: Syntheticoligonucleotide"
tgaaacacgcttgtctaattcgat

SEQ ID NO: 1374 - DNA, 24 bp, Artificial Sequence
  source: /note="Description of Artificial Sequence: Syntheticoligonucleotide"
aatcagcattaactctttaacgcc

SEQ ID NO: 1375 - DNA, 24 bp, Artificial Sequence
  source: /note="Description of Artificial Sequence: Syntheticoligonucleotide"
actctcgactttatatcacgccag

SEQ ID NO: 1376 - DNA, 24 bp, Artificial Sequence
  source: /note="Description of Artificial Sequence: Syntheticoligonucleotide"
ccttatctcagatttgttcggcga

SEQ ID NO: 1377 - DNA, 24 bp, Artificial Sequence
  source: /note="Description of Artificial Sequence: Syntheticoligonucleotide"
tcactaacctgacagcaaccatac

SEQ ID NO: 1378 - DNA, 24 bp, Artificial Sequence
  source: /note="Description of Artificial Sequence: Syntheticoligonucleotide"
tcagcaacgccactaaagctatat

SEQ ID NO: 1379 - DNA, 24 bp, Artificial Sequence
  source: /note="Description of Artificial Sequence: Syntheticoligonucleotide"
cggcaggccctaaagaaatttgta

SEQ ID NO: 1380 - DNA, 24 bp, Artificial Sequence
  source: /note="Description of Artificial Sequence: Syntheticoligonucleotide"
ctagttgtgtactatcatatcgac

SEQ ID NO: 1381 - DNA, 24 bp, Artificial Sequence
  source: /note="Description of Artificial Sequence: Syntheticoligonucleotide"
gcctactatatggtgcgacaataa

SEQ ID NO: 1382 - DNA, 24 bp, Artificial Sequence
  source: /note="Description of Artificial Sequence: Syntheticoligonucleotide"
gcgacgtcaactggaagatgtatg

SEQ ID NO: 1383 - DNA, 24 bp, Artificial Sequence
  source: /note="Description of Artificial Sequence: Syntheticoligonucleotide"
cctggatatgttcagtttatcgct

SEQ ID NO: 1384 - DNA, 24 bp, Artificial Sequence
  source: /note="Description of Artificial Sequence: Syntheticoligonucleotide"
ttcttagcccatggttgggtgaaa

SEQ ID NO: 1385 - DNA, 24 bp, Artificial Sequence
  source: /note="Description of Artificial Sequence: Syntheticoligonucleotide"
taccaagatctgttgattacgcag

SEQ ID NO: 1386 - DNA, 24 bp, Artificial Sequence
  source: /note="Description of Artificial Sequence: Syntheticoligonucleotide"
taactgggactaccacaattatca

SEQ ID NO: 1387 - DNA, 24 bp, Artificial Sequence
  source: /note="Description of Artificial Sequence: Syntheticoligonucleotide"
cgcaagtgtacctggattgagcat

SEQ ID NO: 1388 - DNA, 24 bp, Artificial Sequence
  source: /note="Description of Artificial Sequence: Syntheticoligonucleotide"
cgttgtccactttctggaactgat

SEQ ID NO: 1389 - DNA, 24 bp, Artificial Sequence
  source: /note="Description of Artificial Sequence: Syntheticoligonucleotide"
ttatgagcccgcacgattattata

SEQ ID NO: 1390 - DNA, 24 bp, Artificial Sequence
  source: /note="Description of Artificial Sequence: Syntheticoligonucleotide"
gctgtgttgcggtgcatctaagta

SEQ ID NO: 1391 - DNA, 24 bp, Artificial Sequence
  source: /note="Description of Artificial Sequence: Syntheticoligonucleotide"
aaataagcaggtgccactcattga

SEQ ID NO: 1392 - DNA, 24 bp, Artificial Sequence
  source: /note="Description of Artificial Sequence: Syntheticoligonucleotide"
gacatcgaagttcaaagcgctccc

SEQ ID NO: 1393 - DNA, 24 bp, Artificial Sequence
  source: /note="Description of Artificial Sequence: Syntheticoligonucleotide"
tctgtcattcatctccttagtcag

SEQ ID NO: 1394 - DNA, 24 bp, Artificial Sequence
  source: /note="Description of Artificial Sequence: Syntheticoligonucleotide"
taaccacagtaggacggactaaca

SEQ ID NO: 1395 - DNA, 24 bp, Artificial Sequence
  source: /note="Description of Artificial Sequence: Syntheticoligonucleotide"
gttccgaggttccgaatgcgaggt

SEQ ID NO: 1396 - DNA, 24 bp, Artificial Sequence
  source: /note="Description of Artificial Sequence: Syntheticoligonucleotide"
ttcaagattccaattctgcgaagc

SEQ ID NO: 1397 - DNA, 24 bp, Artificial Sequence
  source: /note="Description of Artificial Sequence: Syntheticoligonucleotide"
tgcttggtcttgggacttacaagc

SEQ ID NO: 1398 - DNA, 24 bp, Artificial Sequence
  source: /note="Description of Artificial Sequence: Syntheticoligonucleotide"
ggagcctacatctcgctaactcct

SEQ ID NO: 1399 - DNA, 24 bp, Artificial Sequence
  source: /note="Description of Artificial Sequence: Syntheticoligonucleotide"
acaggagtaatggcagggttgtgt

SEQ ID NO: 1400 - DNA, 24 bp, Artificial Sequence
  source: /note="Description of Artificial Sequence: Syntheticoligonucleotide"
tcacgattgaacaaactcattcga

SEQ ID NO: 1401 - DNA, 24 bp, Artificial Sequence
  source: /note="Description of Artificial Sequence: Syntheticoligonucleotide"
gtgttccagccacgacgttaagct

SEQ ID NO: 1402 - DNA, 24 bp, Artificial Sequence
  source: /note="Description of Artificial Sequence: Syntheticoligonucleotide"
tagtggtaagagagagtgaagcga

SEQ ID NO: 1403 - DNA, 24 bp, Artificial Sequence
  source: /note="Description of Artificial Sequence: Syntheticoligonucleotide"
gtatgctaattgggaaggccaggt

SEQ ID NO: 1404 - DNA, 24 bp, Artificial Sequence
  source: /note="Description of Artificial Sequence: Syntheticoligonucleotide"
gctctaactcaatctcagggataa

SEQ ID NO: 1405 - DNA, 24 bp, Artificial Sequence
  source: /note="Description of Artificial Sequence: Syntheticoligonucleotide"
ttccaaactggtaaactgctcgga

SEQ ID NO: 1406 - DNA, 24 bp, Artificial Sequence
  source: /note="Description of Artificial Sequence: Syntheticoligonucleotide"
attcaagcagctcatcttgccagg

SEQ ID NO: 1407 - DNA, 24 bp, Artificial Sequence
  source: /note="Description of Artificial Sequence: Syntheticoligonucleotide"
tctcctgtacatcgttgacagcgt

SEQ ID NO: 1408 - DNA, 24 bp, Artificial Sequence
  source: /note="Description of Artificial Sequence: Syntheticoligonucleotide"
tcgtaggtttcgtgctaagtgccg

SEQ ID NO: 1409 - DNA, 24 bp, Artificial Sequence
  source: /note="Description of Artificial Sequence: Syntheticoligonucleotide"
gagcaaagagactcatcgtctatc

SEQ ID NO: 1410 - DNA, 24 bp, Artificial Sequence
  source: /note="Description of Artificial Sequence: Syntheticoligonucleotide"
acggtgtagttattagggtcctag

SEQ ID NO: 1411 - DNA, 24 bp, Artificial Sequence
  source: /note="Description of Artificial Sequence: Syntheticoligonucleotide"
ttggattctttcgacaccaaggcg

SEQ ID NO: 1412 - DNA, 24 bp, Artificial Sequence
  source: /note="Description of Artificial Sequence: Syntheticoligonucleotide"
tactgcacgtagatggcgtgtcta

SEQ ID NO: 1413 - DNA, 24 bp, Artificial Sequence
  source: /note="Description of Artificial Sequence: Syntheticoligonucleotide"
caaagggcttagcttattgcagcc

SEQ ID NO: 1414 - DNA, 24 bp, Artificial Sequence
  source: /note="Description of Artificial Sequence: Syntheticoligonucleotide"
caatgcaatgatgggtcgcggtct

SEQ ID NO: 1415 - DNA, 24 bp, Artificial Sequence
  source: /note="Description of Artificial Sequence: Syntheticoligonucleotide"
ttgacgccaacaagacgcgggtga

SEQ ID NO: 1416 - DNA, 24 bp, Artificial Sequence
  source: /note="Description of Artificial Sequence: Syntheticoligonucleotide"
gagtacagccacagggatgcatgc

SEQ ID NO: 1417 - DNA, 24 bp, Artificial Sequence
  source: /note="Description of Artificial Sequence: Syntheticoligonucleotide"
acaacaatgcttctgccgctttac

SEQ ID NO: 1418 - DNA, 24 bp, Artificial Sequence
  source: /note="Description of Artificial Sequence: Syntheticoligonucleotide"
tttgggtcagtgaggcagtgagca

SEQ ID NO: 1419 - DNA, 24 bp, Artificial Sequence
  source: /note="Description of Artificial Sequence: Syntheticoligonucleotide"
cgatgtgcgtggcgcgacttcatg

SEQ ID NO: 1420 - DNA, 24 bp, Artificial Sequence
  source: /note="Description of Artificial Sequence: Syntheticoligonucleotide"
tcaagcaatgcacaagttctcggt

SEQ ID NO: 1421 - DNA, 24 bp, Artificial Sequence
  source: /note="Description of Artificial Sequence: Syntheticoligonucleotide"
ggcaagaccctagaataacaaatt

SEQ ID NO: 1422 - DNA, 24 bp, Artificial Sequence
  source: /note="Description of Artificial Sequence: Syntheticoligonucleotide"
atcacgttctacctctcacaagta

SEQ ID NO: 1423 - DNA, 24 bp, Artificial Sequence
  source: /note="Description of Artificial Sequence: Syntheticoligonucleotide"
ttcaccttagtgtcttagactccc

SEQ ID NO: 1424 - DNA, 24 bp, Artificial Sequence
  source: /note="Description of Artificial Sequence: Syntheticoligonucleotide"
ggacgacaagtgcatatactacag

SEQ ID NO: 1425 - DNA, 24 bp, Artificial Sequence
  source: /note="Description of Artificial Sequence: Syntheticoligonucleotide"
aagtgtttgggagaggtgtcatcg

SEQ ID NO: 1426 - DNA, 24 bp, Artificial Sequence
  source: /note="Description of Artificial Sequence: Syntheticoligonucleotide"
aaataacgagggatcgtaacgaca

SEQ ID NO: 1427 - DNA, 24 bp, Artificial Sequence
  source: /note="Description of Artificial Sequence: Syntheticoligonucleotide"
taggtttacctttctcttgcagag

SEQ ID NO: 1428 - DNA, 24 bp, Artificial Sequence
  source: /note="Description of Artificial Sequence: Syntheticoligonucleotide"
ctcatacccggtgttgaagaccaa

SEQ ID NO: 1429 - DNA, 24 bp, Artificial Sequence
  source: /note="Description of Artificial Sequence: Syntheticoligonucleotide"
gagacaccacctcaatgctaagcg

SEQ ID NO: 1430 - DNA, 24 bp, Artificial Sequence
  source: /note="Description of Artificial Sequence: Syntheticoligonucleotide"
ctcgcttatattcccgatgctggc

SEQ ID NO: 1431 - DNA, 24 bp, Artificial Sequence
  source: /note="Description of Artificial Sequence: Syntheticoligonucleotide"
tagatttgccagcgttcgccttgg

SEQ ID NO: 1432 - DNA, 24 bp, Artificial Sequence
  source: /note="Description of Artificial Sequence: Syntheticoligonucleotide"
aatgctgcatagtgcgcttgtcgg

SEQ ID NO: 1433 - DNA, 24 bp, Artificial Sequence
  source: /note="Description of Artificial Sequence: Syntheticoligonucleotide"
tgcactgtttatgatctgtaccaa

SEQ ID NO: 1434 - DNA, 24 bp, Artificial Sequence
  source: /note="Description of Artificial Sequence: Syntheticoligonucleotide"
gttcctgtccacaccgcttcatgt

SEQ ID NO: 1435 - DNA, 24 bp, Artificial Sequence
  source: /note="Description of Artificial Sequence: Syntheticoligonucleotide"
gatggccgagacttcctgggatca

SEQ ID NO: 1436 - DNA, 24 bp, Artificial Sequence
  source: /note="Description of Artificial Sequence: Syntheticoligonucleotide"
tgtggcagggatatggtcacattt

SEQ ID NO: 1437 - DNA, 24 bp, Artificial Sequence
  source: /note="Description of Artificial Sequence: Syntheticoligonucleotide"
aggaggtttagtataaattgggtg

SEQ ID NO: 1438 - DNA, 24 bp, Artificial Sequence
  source: /note="Description of Artificial Sequence: Syntheticoligonucleotide"
tgacgcttatcggcagtcttgaaa

SEQ ID NO: 1439 - DNA, 24 bp, Artificial Sequence
  source: /note="Description of Artificial Sequence: Syntheticoligonucleotide"
gataagacgtgtgcttccgttaga

SEQ ID NO: 1440 - DNA, 24 bp, Artificial Sequence
  source: /note="Description of Artificial Sequence: Syntheticoligonucleotide"
ctgctcgggcatagcggaacctta

SEQ ID NO: 1441 - DNA, 24 bp, Artificial Sequence
  source: /note="Description of Artificial Sequence: Syntheticoligonucleotide"
ttgcagacagtccgctccgaatat

SEQ ID NO: 1442 - DNA, 24 bp, Artificial Sequence
  source: /note="Description of Artificial Sequence: Syntheticoligonucleotide"
cacatctacccgcaattcggtcgt

SEQ ID NO: 1443 - DNA, 24 bp, Artificial Sequence
  source: /note="Description of Artificial Sequence: Syntheticoligonucleotide"
gatacacgccaaatattggagggc

SEQ ID NO: 1444 - DNA, 24 bp, Artificial Sequence
  source: /note="Description of Artificial Sequence: Syntheticoligonucleotide"
cgacgacgagtgtccgcttgacta

SEQ ID NO: 1445 - DNA, 24 bp, Artificial Sequence
  source: /note="Description of Artificial Sequence: Syntheticoligonucleotide"
gtccatatgcatcaactatggaaa

SEQ ID NO: 1446 - DNA, 24 bp, Artificial Sequence
  source: /note="Description of Artificial Sequence: Syntheticoligonucleotide"
ttgagtatctttgatagggacctc

SEQ ID NO: 1447 - DNA, 24 bp, Artificial Sequence
  source: /note="Description of Artificial Sequence: Syntheticoligonucleotide"
aatgtctgtcaagcagtttatctc

SEQ ID NO: 1448 - DNA, 24 bp, Artificial Sequence
  source: /note="Description of Artificial Sequence: Syntheticoligonucleotide"
ctctgtgaaaggaagtatattgag

SEQ ID NO: 1449 - DNA, 24 bp, Artificial Sequence
  source: /note="Description of Artificial Sequence: Syntheticoligonucleotide"
tataaacacccatgtgctggtctg

SEQ ID NO: 1450 - DNA, 24 bp, Artificial Sequence
  source: /note="Description of Artificial Sequence: Syntheticoligonucleotide"
ctgaccaaagcgatagacctacaa

SEQ ID NO: 1451 - DNA, 24 bp, Artificial Sequence
  source: /note="Description of Artificial Sequence: Syntheticoligonucleotide"
tttaccacctagctcgcgaaatca

SEQ ID NO: 1452 - DNA, 24 bp, Artificial Sequence
  source: /note="Description of Artificial Sequence: Syntheticoligonucleotide"
gcgggtcacaatctgatgtacagg

SEQ ID NO: 1453 - DNA, 24 bp, Artificial Sequence
  source: /note="Description of Artificial Sequence: Syntheticoligonucleotide"
ggtccaacttcgctcaagcttcta

SEQ ID NO: 1454 - DNA, 24 bp, Artificial Sequence
  source: /note="Description of Artificial Sequence: Syntheticoligonucleotide"
atcattagcgttaactcgtgccct

SEQ ID NO: 1455 - DNA, 24 bp, Artificial Sequence
  source: /note="Description of Artificial Sequence: Syntheticoligonucleotide"
accgtttgaggccgctctttaact

SEQ ID NO: 1456 - DNA, 24 bp, Artificial Sequence
  source: /note="Description of Artificial Sequence: Syntheticoligonucleotide"
aggtacgcaggtatcatatcacta

SEQ ID NO: 1457 - DNA, 24 bp, Artificial Sequence
  source: /note="Description of Artificial Sequence: Syntheticoligonucleotide"
cgataacgtagattagtttggcac

SEQ ID NO: 1458 - DNA, 24 bp, Artificial Sequence
  source: /note="Description of Artificial Sequence: Syntheticoligonucleotide"
agcctattacataagtctctcatc

SEQ ID NO: 1459 - DNA, 24 bp, Artificial Sequence
  source: /note="Description of Artificial Sequence: Syntheticoligonucleotide"
caacgtgttagttgaatggtcgcc

SEQ ID NO: 1460 - DNA, 24 bp, Artificial Sequence
  source: /note="Description of Artificial Sequence: Syntheticoligonucleotide"
gagaagaatacagcgaaataagga

SEQ ID NO: 1461 - DNA, 24 bp, Artificial Sequence
  source: /note="Description of Artificial Sequence: Syntheticoligonucleotide"
tacaagagtagtgaatcctcagtc

SEQ ID NO: 1462 - DNA, 24 bp, Artificial Sequence
  source: /note="Description of Artificial Sequence: Syntheticoligonucleotide"
ttccgcatcgtgattgcttgctaa

SEQ ID NO: 1463 - DNA, 24 bp, Artificial Sequence
  source: /note="Description of Artificial Sequence: Syntheticoligonucleotide"
tgtttaccagaggctatgtccaac

SEQ ID NO: 1464 - DNA, 24 bp, Artificial Sequence
  source: /note="Description of Artificial Sequence: Syntheticoligonucleotide"
gacttagaataacgtttgcaacta

SEQ ID NO: 1465 - DNA, 24 bp, Artificial Sequence
  source: /note="Description of Artificial Sequence: Syntheticoligonucleotide"
ttactgggtaatggcctcgtatca

SEQ ID NO: 1466 - DNA, 24 bp, Artificial Sequence
  source: /note="Description of Artificial Sequence: Syntheticoligonucleotide"
acctgtttcgtaagcagctacatc

SEQ ID NO: 1467 - DNA, 24 bp, Artificial Sequence
  source: /note="Description of Artificial Sequence: Syntheticoligonucleotide"
tttagtcaatagtgtggtgctatt

SEQ ID NO: 1468 - DNA, 24 bp, Artificial Sequence
  source: /note="Description of Artificial Sequence: Syntheticoligonucleotide"
tgggctatctcagtgacatctagg

SEQ ID NO: 1469 - DNA, 24 bp, Artificial Sequence
  source: /note="Description of Artificial Sequence: Syntheticoligonucleotide"
aggatgtcggtaggccagtcctta

SEQ ID NO: 1470 - DNA, 24 bp, Artificial Sequence
  source: /note="Description of Artificial Sequence: Syntheticoligonucleotide"
caatcgcatggagtcgcagtcgta

SEQ ID NO: 1471 - DNA, 24 bp, Artificial Sequence
  source: /note="Description of Artificial Sequence: Syntheticoligonucleotide"
aattgtctataccggcgattaaca

SEQ ID NO: 1472 - DNA, 24 bp, Artificial Sequence
  source: /note="Description of Artificial Sequence: Syntheticoligonucleotide"
tgatgaggctcctaagaaatacgt

SEQ ID NO: 1473 - DNA, 24 bp, Artificial Sequence
  source: /note="Description of Artificial Sequence: Syntheticoligonucleotide"
ggcgttacaaattgaccggacagg

SEQ ID NO: 1474 - DNA, 24 bp, Artificial Sequence
  source: /note="Description of Artificial Sequence: Syntheticoligonucleotide"
atccctaattggtggcccgcgttt

SEQ ID NO: 1475 - DNA, 24 bp, Artificial Sequence
  source: /note="Description of Artificial Sequence: Syntheticoligonucleotide"
cggtcgttaaacgtctgtggttgg

SEQ ID NO: 1476 - DNA, 24 bp, Artificial Sequence
  source: /note="Description of Artificial Sequence: Syntheticoligonucleotide"
ataactgtcggcctctatgtcgca

SEQ ID NO: 1477 - DNA, 24 bp, Artificial Sequence
  source: /note="Description of Artificial Sequence: Syntheticoligonucleotide"
cgtggctgacgtctgatttcacct

SEQ ID NO: 1478 - DNA, 24 bp, Artificial Sequence
  source: /note="Description of Artificial Sequence: Syntheticoligonucleotide"
ctagttacccttcggaattaatga

SEQ ID NO: 1479 - DNA, 24 bp, Artificial Sequence
  source: /note="Description of Artificial Sequence: Syntheticoligonucleotide"
gacacgatggcttccatataagac

SEQ ID NO: 1480 - DNA, 24 bp, Artificial Sequence
  source: /note="Description of Artificial Sequence: Syntheticoligonucleotide"
ttcgcgctactagttgtcaaagcc

SEQ ID NO: 1481 - DNA, 24 bp, Artificial Sequence
  source: /note="Description of Artificial Sequence: Syntheticoligonucleotide"
agggtctgttctagcggctcagta

SEQ ID NO: 1482 - DNA, 24 bp, Artificial Sequence
  source: /note="Description of Artificial Sequence: Syntheticoligonucleotide"
tggttgtgaaattgcatcactggg

SEQ ID NO: 1483 - DNA, 24 bp, Artificial Sequence
  source: /note="Description of Artificial Sequence: Syntheticoligonucleotide"
cgtgcctaggtattgccacaactt

SEQ ID NO: 1484 - DNA, 24 bp, Artificial Sequence
  source: /note="Description of Artificial Sequence: Syntheticoligonucleotide"
cacggctatgaggtatgtctgact

SEQ ID NO: 1485 - DNA, 24 bp, Artificial Sequence
  source: /note="Description of Artificial Sequence: Syntheticoligonucleotide"
tctaggttcgatgataacgggcgt

SEQ ID NO: 1486 - DNA, 24 bp, Artificial Sequence
  source: /note="Description of Artificial Sequence: Syntheticoligonucleotide"
acactcgctgccatttggccttaa

SEQ ID NO: 1487 - DNA, 24 bp, Artificial Sequence
  source: /note="Description of Artificial Sequence: Syntheticoligonucleotide"
gcggattaaagcgtgttgtgttga

SEQ ID NO: 1488 - DNA, 24 bp, Artificial Sequence
  source: /note="Description of Artificial Sequence: Syntheticoligonucleotide"
aattgtgtgagtcggtccgttgca

SEQ ID NO: 1489 - DNA, 36 bp, Artificial Sequence; identical to SEQ ID NO: 1
  source: /note="Description of Artificial Sequence: Syntheticoligonucleotide"

SEQ ID NO: 1490 - DNA, 36 bp, Artificial Sequence; identical to SEQ ID NO: 2
  source: /note="Description of Artificial Sequence: Syntheticoligonucleotide"

SEQ ID NO: 1491 - DNA, 36 bp, Artificial Sequence; identical to SEQ ID NO: 3
  source: /note="Description of Artificial Sequence: Syntheticoligonucleotide"

SEQ ID NO: 1492 - DNA, 36 bp, Artificial Sequence; identical to SEQ ID NO: 4
  source: /note="Description of Artificial Sequence: Syntheticoligonucleotide"

SEQ ID NO: 1493 - DNA, 36 bp, Artificial Sequence; identical to SEQ ID NO: 5
  source: /note="Description of Artificial Sequence: Syntheticoligonucleotide"

SEQ ID NO: 1494 - DNA, 36 bp, Artificial Sequence; identical to SEQ ID NO: 6
  source: /note="Description of Artificial Sequence: Syntheticoligonucleotide"

SEQ ID NO: 1495 - DNA, 36 bp, Artificial Sequence; identical to SEQ ID NO: 7
  source: /note="Description of Artificial Sequence: Syntheticoligonucleotide"

SEQ ID NO: 1496 - DNA, 36 bp, Artificial Sequence; identical to SEQ ID NO: 8
  source: /note="Description of Artificial Sequence: Syntheticoligonucleotide"

SEQ ID NO: 1497 - DNA, 36 bp, Artificial Sequence; identical to SEQ ID NO: 9
  source: /note="Description of Artificial Sequence: Syntheticoligonucleotide"

SEQ ID NO: 1498 - DNA, 36 bp, Artificial Sequence; identical to SEQ ID NO: 10
  source: /note="Description of Artificial Sequence: Syntheticoligonucleotide"

SEQ ID NO: 1499 - DNA, 70 bp, Artificial Sequence
  source: /note="Description of Artificial Sequence: Syntheticoligonucleotide"
tctctcatggcactgtactcttcttgtccagctgtatccagtatgtccaacaaacaggtttcaccatcta

SEQ ID NO: 1500 - DNA, 70 bp, Artificial Sequence
  source: /note="Description of Artificial Sequence: Syntheticoligonucleotide"
tctctcatggcactgtactcttcttttccagctgtatccagtatgtccaacaaacaggtttcaccatcta

SEQ ID NO: 1501 - DNA, 22 bp, Artificial Sequence
  source: /note="Description of Artificial Sequence: Syntheticprobe"
acatactggatacagctggacw

SEQ ID NO: 1502 - DNA, 21 bp, Artificial Sequence
  source: /note="Description of Artificial Sequence: Syntheticprobe"
agaagagtacagtgccatgag

SEQ ID NO: 1503 - DNA, 42 bp, Artificial Sequence
  source: /note="Description of Artificial Sequence: Syntheticprobe"
gtggcaacagaaatcaggtggtgacactcttgcctacgccac

SEQ ID NO: 1504 - DNA, 42 bp, Artificial Sequence
  source: /note="Description of Artificial Sequence: Syntheticprobe"
agaggactgctaaaggtttgtaggcactcttgcctacgccat

SEQ ID NO: 1505 - DNA, 21 bp, Artificial Sequence
  source: /note="Description of Artificial Sequence: Syntheticprobe"
cagctccaactaccacaagtt

SEQ ID NO: 1506 - DNA, 60 bp, Artificial Sequence
  source: /note="Description of Artificial Sequence: Syntheticoligonucleotide"
actgaatataaacttgtggtagttggagctggtggcgtaggcaagagtgccttgacgata

SEQ ID NO: 1507 - DNA, 60 bp, Artificial Sequence
  source: /note="Description of Artificial Sequence: Syntheticoligonucleotide"
actgaatataaacttgtggtagttggagctgatggcgtaggcaagagtgccttgacgata

SEQ ID NO: 1508 - DNA, 18 bp, Artificial Sequence
  source: /note="Description of Artificial Sequence: Syntheticprobe"
cactcttgcctacgccay

SEQ ID NO: 1509 - DNA, 21 bp, Artificial Sequence
  source: /note="Description of Artificial Sequence: Syntheticprobe"
cagctccaactaccacaagtt

SEQ ID NO: 1510 - DNA, 50 bp, Artificial Sequence
  source: /note="Description of Artificial Sequence: Syntheticprobe"
ctaatagctcctgtgccctcgtatctccatagaaaatctttctcctgctc

SEQ ID NO: 1511 - DNA, 50 bp, Artificial Sequence
  source: /note="Description of Artificial Sequence: Syntheticprobe"
aatccgtcgactagcctgagaattctccatagaaaatctttctcctgctt

SEQ ID NO: 1512 - DNA, 24 bp, Artificial Sequence
  source: /note="Description of Artificial Sequence: Syntheticprobe"
agtgatttcagagagaggatctcg

SEQ ID NO: 1513 - DNA, 71 bp, Artificial Sequence
  source: /note="Description of Artificial Sequence: Syntheticoligonucleotide"
aatttctacacgagatcctctctctgaaatcactgagcaggagaaagattttctatggagtcacaggtaag

SEQ ID NO: 1514 - DNA, 71 bp, Artificial Sequence
  source: /note="Description of Artificial Sequence: Syntheticoligonucleotide"
aatttctacacgagatcctctctctgaaatcactaagcaggagaaagattttctatggagtcacaggtaag

SEQ ID NO: 1515 - DNA, 26 bp, Artificial Sequence
  source: /note="Description of Artificial Sequence: Syntheticprobe"
ctccatagaaaatctttctcctgcty

SEQ ID NO: 1516 - DNA, 24 bp, Artificial Sequence
  source: /note="Description of Artificial Sequence: Syntheticprobe"
agtgatttcagagagaggatctcg

SEQ ID NO: 1517 - DNA, 50 bp, Artificial Sequence
  source: /note="Description of Artificial Sequence: Syntheticprobe"
tagatgccgctgcaggtatggaaaggtgctttatttttaggtacttctcg

SEQ ID NO: 1518 - DNA, 50 bp, Artificial Sequence
  source: /note="Description of Artificial Sequence: Syntheticprobe"
cgtaccattgaatctggagaccttggtgctttatttttaggtacttctca

SEQ ID NO: 1519 - DNA, 21 bp, Artificial Sequence
  source: /note="Description of Artificial Sequence: Syntheticprobe"
cttggtttgagctgtttgagg

SEQ ID NO: 1520 - DNA, 70 bp, Artificial Sequence
  source: /note="Description of Artificial Sequence: Syntheticoligonucleotide"
tccaccacctcctcaaacagctcaaaccaagcgagaagtacctaaaaataaagcacctactgctgaaaag

SEQ ID NO: 1521 - DNA, 70 bp, Artificial Sequence
  source: /note="Description of Artificial Sequence: Syntheticoligonucleotide"
tccaccacctcctcaaacagctcaaaccaagtgagaagtacctaaaaataaagcacctactgctgaaaag

SEQ ID NO: 1522 - DNA, 26 bp, Artificial Sequence
  source: /note="Description of Artificial Sequence: Syntheticprobe"
ggtgctttatttttaggtacttctcr

SEQ ID NO: 1523 - DNA, 21 bp, Artificial Sequence
  source: /note="Description of Artificial Sequence: Syntheticprobe"
cttggtttgagctgtttgagg

SEQ ID NO: 1524 - DNA, 46 bp, Artificial Sequence
  source: /note="Description of Artificial Sequence: Syntheticprobe"
ctaatagctcctgtgccctcgtatacatactggatacagctggaca

SEQ ID NO: 1525 - DNA, 46 bp, Artificial Sequence
  source: /note="Description of Artificial Sequence: Syntheticprobe"
aatccgtcgactagcctgagaattacatactggatacagctggact

SEQ ID NO: 1526 - DNA, 21 bp, Artificial Sequence
  source: /note="Description of Artificial Sequence: Syntheticprobe"
agaagagtacagtgccatgag

SEQ ID NO: 1527 - DNA, 70 bp, Artificial Sequence
  source: /note="Description of Artificial Sequence: Syntheticoligonucleotide"
tctctcatggcactgtactcttctagtccagctgtatccagtatgtccaacaaacaggtttcaccatcta

SEQ ID NO: 1528 - DNA, 46 bp, Artificial Sequence
  source: /note="Description of Artificial Sequence: Syntheticprobe"
actggtaacccagacatgatcggtacatactggatacagctggaca

SEQ ID NO: 1529 - DNA, 46 bp, Artificial Sequence
  source: /note="Description of Artificial Sequence: Syntheticprobe"
actccctgtgggtgagcttaatggacatactggatacagctggact

SEQ ID NO: 1530 - DNA, 40 bp, Artificial Sequence
  source: /note="Description of Artificial Sequence: Syntheticprobe"
catttggtcattggttcaagacgatcatggtgggggcagc

SEQ ID NO: 1531 - DNA, 40 bp, Artificial Sequence
  source: /note="Description of Artificial Sequence: Syntheticprobe"
ctggtccgttgtggtccttctaactcatggtgggggcagt

SEQ ID NO: 1532 - DNA, 18 bp, Artificial Sequence
  source: /note="Description of Artificial Sequence: Syntheticprobe"
gcctcacaacctccgtca

SEQ ID NO: 1533 - DNA, 53 bp, Artificial Sequence
  source: /note="Description of Artificial Sequence: Syntheticoligonucleotide"
cagcacatgacggaggttgtgaggcgctgcccccaccatgagcgctgctcaga

SEQ ID NO: 1534 - DNA, 53 bp, Artificial Sequence
  source: /note="Description of Artificial Sequence: Syntheticoligonucleotide"
cagcacatgacggaggttgtgaggcactgcccccaccatgagcgctgctcaga

SEQ ID NO: 1535 - DNA, 16 bp, Artificial Sequence
  source: /note="Description of Artificial Sequence: Syntheticprobe"
tcatggtgggggcagy

SEQ ID NO: 1536 - DNA, 18 bp, Artificial Sequence
  source: /note="Description of Artificial Sequence: Syntheticprobe"
gcctcacaacctccgtca

SEQ ID NO: 1537 - DNA, 47 bp, Artificial Sequence
  source: /note="Description of Artificial Sequence: Syntheticprobe"
gctcccgttaatgctcccgttaataggtgattttggtctagctacag

SEQ ID NO: 1538 - DNA, 45 bp, Artificial Sequence
  source: /note="Description of Artificial Sequence: Syntheticprobe"
tagcaagggaaatagcaagggaaagacatactggatacagctgga

SEQ ID NO: 1539 - DNA, 45 bp, Artificial Sequence
  source: /note="Description of Artificial Sequence: Syntheticprobe"
tggtgaattagctggtgaattagcacatactggatacagctggac

SEQ ID NO: 1540 - DNA, 52 bp, Artificial Sequence
  source: /note="Description of Artificial Sequence: Syntheticprobe"
actggtaacccagacatgatcggtctggcaccattaaagaaaatatcatctt

SEQ ID NO: 1541 - DNA, 51 bp, Artificial Sequence
  source: /note="Description of Artificial Sequence: Syntheticprobe"
actccctgtgggtgagcttaatgggcctggcaccattaaagaaaatatcat

SEQ ID NO: 1542 - DNA, 30 bp, Artificial Sequence
  source: /note="Description of Artificial Sequence: Syntheticprobe"
tggtgtttcctatgatgaatatagatacag

SEQ ID NO: 1543 - DNA, 48 bp, Artificial Sequence
  source: /note="Description of Artificial Sequence: Syntheticprobe"
ctaatagctcctgtgccctcgtatactcagtgtgattccaccttctcc

SEQ ID NO: 1544 - DNA, 48 bp, Artificial Sequence
  source: /note="Description of Artificial Sequence: Syntheticprobe"
aatccgtcgactagcctgagaattactcagtgtgattccaccttctca

SEQ ID NO: 1545 - DNA, 28 bp, Artificial Sequence
  source: /note="Description of Artificial Sequence: Syntheticprobe"
aagaactatattgtctttctctgcaaac

SEQ ID NO: 1546 - DNA, 50 bp, Artificial Sequence
  source: /note="Description of Artificial Sequence: Syntheticprobe"
catttggtcattggttcaagacgattaatgctatgcagaaaatcttagag

SEQ ID NO: 1547 - DNA, 51 bp, Artificial Sequence
  source: /note="Description of Artificial Sequence: Syntheticprobe"
ctggtccgttgtggtccttctaactcattaatgctatgcagaaaatcttag

SEQ ID NO: 1548 - DNA, 22 bp, Artificial Sequence
  source: /note="Description of Artificial Sequence: Syntheticprobe"
tgtcccatctgtctggagttga

SEQ ID NO: 1549 - DNA, 45 bp, Artificial Sequence
  source: /note="Description of Artificial Sequence: Syntheticprobe"
ctaatagctcctgtgccctcgtatcaaagcgagcaagagaatccc

SEQ ID NO: 1550 - DNA, 45 bp, Artificial Sequence
  source: /note="Description of Artificial Sequence: Syntheticprobe"
aatccgtcgactagcctgagaattaaagcgagcaagagaatcccc

SEQ ID NO: 1551 - DNA, 23 bp, Artificial Sequence
  source: /note="Description of Artificial Sequence: Syntheticprobe"
aggacagaaagatcttcaggggg

SEQ ID NO: 1552 - DNA, 47 bp, Artificial Sequence
  source: /note="Description of Artificial Sequence: Syntheticprobe"
actggtaacccagacatgatcggtgtgggatttttagcacagcaagt

SEQ ID NO: 1553 - DNA, 47 bp, Artificial Sequence
  source: /note="Description of Artificial Sequence: Syntheticprobe"
actccctgtgggtgagcttaatggtgtgggatttttagcacagcaag

SEQ ID NO: 1554 - DNA, 25 bp, Artificial Sequence
  source: /note="Description of Artificial Sequence: Syntheticprobe"
ggaaaatctgtccaggtatcagatg

SEQ ID NO: 1555 - DNA, 43 bp, Artificial Sequence
  source: /note="Description of Artificial Sequence: Syntheticprobe"
actggtaacccagacatgatcggtagaaggtgtctgcgggagc

SEQ ID NO: 1556 - DNA, 45 bp, Artificial Sequence
  source: /note="Description of Artificial Sequence: Syntheticprobe"
actggtaacccagacatgatcggtgctgcgtgatgatgaaatcgg

SEQ ID NO: 1557 - DNA, 43 bp, Artificial Sequence
  source: /note="Description of Artificial Sequence: Syntheticprobe"
actccctgtgggtgagcttaatggagaaggtgtctgcgggagt

SEQ ID NO: 1558 - DNA, 45 bp, Artificial Sequence
  source: /note="Description of Artificial Sequence: Syntheticprobe"
actccctgtgggtgagcttaatgggctgcgtgatgatgaaatcga

SEQ ID NO: 1559 - DNA, 42 bp, Artificial Sequence
  source: /note="Description of Artificial Sequence: Syntheticprobe"
catttggtcattggttcaagacgagacagcgagcagctgagc

SEQ ID NO: 1560 - DNA, 41 bp, Artificial Sequence
  source: /note="Description of Artificial Sequence: Syntheticprobe"
catttggtcattggttcaagacgagcgcatcacagtcgcgg

SEQ ID NO: 1561 - DNA, 42 bp, Artificial Sequence
  source: /note="Description of Artificial Sequence: Syntheticprobe"
ctggtccgttgtggtccttctaacgacagcgagcagctgaga

SEQ ID NO: 1562 - DNA, 41 bp, Artificial Sequence
  source: /note="Description of Artificial Sequence: Syntheticprobe"
ctggtccgttgtggtccttctaacgcgcatcacagtcgcgt

SEQ ID NO: 1563 - DNA, 51 bp, Artificial Sequence
  source: /note="Description of Artificial Sequence: Syntheticprobe"
ctaatagctcctgtgccctcgtatgagacatcatcaagtggagagaaatca

SEQ ID NO: 1564 - DNA, 57 bp, Artificial Sequence
  source: /note="Description of Artificial Sequence: Syntheticprobe"
ctaatagctcctgtgccctcgtatctgttataaaatttataatgcagtttaaactat

SEQ ID NO: 1565 - DNA, 51 bp, Artificial Sequence
  source: /note="Description of Artificial Sequence: Syntheticprobe"
aatccgtcgactagcctgagaattgagacatcatcaagtggagagaaatcg

SEQ ID NO: 1566 - DNA, 57 bp, Artificial Sequence
  source: /note="Description of Artificial Sequence: Syntheticprobe"
aatccgtcgactagcctgagaattctgttataaaatttataatgcagtttaaactac

SEQ ID NO: 1567 - DNA, 46 bp, Artificial Sequence
  source: /note="Description of Artificial Sequence: Syntheticprobe"
gtggcaacagaaatcaggtggtgacatcatcaaagccccaggctac

SEQ ID NO: 1568 - DNA, 47 bp, Artificial Sequence
  source: /note="Description of Artificial Sequence: Syntheticprobe"
gtggcaacagaaatcaggtggtgaagttgtacttgatgtcgtgtttg

SEQ ID NO: 1569 - DNA, 46 bp, Artificial Sequence
  source: /note="Description of Artificial Sequence: Syntheticprobe"
agaggactgctaaaggtttgtaggcatcatcaaagccccaggctat

SEQ ID NO: 1570 - DNA, 47 bp, Artificial Sequence
  source: /note="Description of Artificial Sequence: Syntheticprobe"
agaggactgctaaaggtttgtaggagttgtacttgatgtcgtgttta

SEQ ID NO: 1571 - DNA, 49 bp, Artificial Sequence
  source: /note="Description of Artificial Sequence: Syntheticprobe"
tagatgccgctgcaggtatggaaaccaatgtggtataagttttctgttt

SEQ ID NO: 1572 - DNA, 48 bp, Artificial Sequence
  source: /note="Description of Artificial Sequence: Syntheticprobe"
tagatgccgctgcaggtatggaaattactatctgtcagggcctttcta

SEQ ID NO: 1573 - DNA, 49 bp, Artificial Sequence
  source: /note="Description of Artificial Sequence: Syntheticprobe"
cgtaccattgaatctggagaccttccaatgtggtataagttttctgttc

SEQ ID NO: 1574 - DNA, 48 bp, Artificial Sequence
  source: /note="Description of Artificial Sequence: Syntheticprobe"
cgtaccattgaatctggagaccttttactatctgtcagggcctttctg

SEQ ID NO: 1575 - DNA, 50 bp, Artificial Sequence
  source: /note="Description of Artificial Sequence: Syntheticprobe"
actggtaacccagacatgatcggtagaacaatcaatgctgttattactgt

SEQ ID NO: 1576 - DNA, 45 bp, Artificial Sequence
  source: /note="Description of Artificial Sequence: Syntheticprobe"
actggtaacccagacatgatcggtccacatcaatcatggtgggaa

SEQ ID NO: 1577 - DNA, 50 bp, Artificial Sequence
  source: /note="Description of Artificial Sequence: Syntheticprobe"
actccctgtgggtgagcttaatggagaacaatcaatgctgttattactga

SEQ ID NO: 1578 - DNA, 45 bp, Artificial Sequence
  source: /note="Description of Artificial Sequence: Syntheticprobe"
actccctgtgggtgagcttaatggccacatcaatcatggtgggat

SEQ ID NO: 1579 - DNA, 52 bp, Artificial Sequence
  source: /note="Description of Artificial Sequence: Syntheticprobe"
ctaatagctcctgtgccctcgtatccaaaacgtgttctcgtgactcagcaat

SEQ ID NO: 1580 - DNA, 51 bp, Artificial Sequence
  source: /note="Description of Artificial Sequence: Syntheticprobe"
ctaatagctcctgtgccctcgtattacaggtaatggattctgacaaggaaa

SEQ ID NO: 1581 - DNA, 52 bp, Artificial Sequence
  source: /note="Description of Artificial Sequence: Syntheticprobe"
aatccgtcgactagcctgagaattccaaaacgtgttctcgtgactcagcaaa

SEQ ID NO: 1582 - DNA, 51 bp, Artificial Sequence
  source: /note="Description of Artificial Sequence: Syntheticprobe"
aatccgtcgactagcctgagaatttacaggtaatggattctgacaaggaat

SEQ ID NO: 1583 - DNA, 48 bp, Artificial Sequence
  source: /note="Description of Artificial Sequence: Syntheticprobe"
gtggcaacagaaatcaggtggtgaagtgggtcctaaaaactcttacat

SEQ ID NO: 1584 - DNA, 49 bp, Artificial Sequence
  source: /note="Description of Artificial Sequence: Syntheticprobe"
gtggcaacagaaatcaggtggtgaccactgtgatcttctatgtatgcaa

SEQ ID NO: 1585 - DNA, 48 bp, Artificial Sequence
  source: /note="Description of Artificial Sequence: Syntheticprobe"
agaggactgctaaaggtttgtaggagtgggtcctaaaaactcttacac

SEQ ID NO: 1586 - DNA, 49 bp, Artificial Sequence
  source: /note="Description of Artificial Sequence: Syntheticprobe"
agaggactgctaaaggtttgtaggccactgtgatcttctatgtatgcag

SEQ ID NO: 1587 - DNA, 45 bp, Artificial Sequence
  source: /note="Description of Artificial Sequence: Syntheticprobe"
tagatgccgctgcaggtatggaaacaccattgtgtgcctatacca

SEQ ID NO: 1588 - DNA, 41 bp, Artificial Sequence
  source: /note="Description of Artificial Sequence: Syntheticprobe"
tagatgccgctgcaggtatggaaaccctggggacaagcact

SEQ ID NO: 1589 - DNA, 45 bp, Artificial Sequence
  source: /note="Description of Artificial Sequence: Syntheticprobe"
cgtaccattgaatctggagaccttcaccattgtgtgcctataccg

SEQ ID NO: 1590 - DNA, 41 bp, Artificial Sequence
  source: /note="Description of Artificial Sequence: Syntheticprobe"
cgtaccattgaatctggagaccttccctggggacaagcacc

SEQ ID NO: 1591 - DNA, 20 bp, Artificial Sequence
  source: /note="Description of Artificial Sequence: Syntheticprobe"
cgatttcatcatcacgcagc

SEQ ID NO: 1592 - DNA, 19 bp, Artificial Sequence
  source: /note="Description of Artificial Sequence: Syntheticprobe"
ctcccgcagacaccttctc

SEQ ID NO: 1593 - DNA, 20 bp, Artificial Sequence
  source: /note="Description of Artificial Sequence: Syntheticprobe"
cgatttcatcatcacgcagc

SEQ ID NO: 1594 - DNA, 19 bp, Artificial Sequence
  source: /note="Description of Artificial Sequence: Syntheticprobe"
ctcccgcagacaccttctc

SEQ ID NO: 1595 - DNA, 18 bp, Artificial Sequence
  source: /note="Description of Artificial Sequence: Syntheticprobe"
cgcgactgtgatgcgcta

SEQ ID NO: 1596 - DNA, 18 bp, Artificial Sequence
  source: /note="Description of Artificial Sequence: Syntheticprobe"
ctcagctgctcgctgtcc

SEQ ID NO: 1597 - DNA, 18 bp, Artificial Sequence
  source: /note="Description of Artificial Sequence: Syntheticprobe"
cgcgactgtgatgcgcta

SEQ ID NO: 1598 - DNA, 18 bp, Artificial Sequence
  source: /note="Description of Artificial Sequence: Syntheticprobe"
ctcagctgctcgctgtcc

SEQ ID NO: 1599 - DNA, 32 bp, Artificial Sequence
  source: /note="Description of Artificial Sequence: Syntheticprobe"
tagtttaaactgcattataaattttataacag

SEQ ID NO: 1600 - DNA, 26 bp, Artificial Sequence
  source: /note="Description of Artificial Sequence: Syntheticprobe"
gatttctctccacttgatgatgtctc

SEQ ID NO: 1601 - DNA, 32 bp, Artificial Sequence; identical to SEQ ID NO: 1599
  source: /note="Description of Artificial Sequence: Syntheticprobe"

SEQ ID NO: 1602 - DNA, 26 bp, Artificial Sequence
  source: /note="Description of Artificial Sequence: Syntheticprobe"
gatttctctccacttgatgatgtctc

SEQ ID NO: 1603 - DNA, 23 bp, Artificial Sequence
  source: /note="Description of Artificial Sequence: Syntheticprobe"
aaacacgacatcaagtacaactg

SEQ ID NO: 1604 - DNA, 20 bp, Artificial Sequence
  source: /note="Description of Artificial Sequence: Syntheticprobe"
tagcctggggctttgatgat

SEQ ID NO: 1605 - DNA, 23 bp, Artificial Sequence
  source: /note="Description of Artificial Sequence: Syntheticprobe"
aaacacgacatcaagtacaactg

SEQ ID NO: 1606 - DNA, 20 bp, Artificial Sequence
  source: /note="Description of Artificial Sequence: Syntheticprobe"
tagcctggggctttgatgat

SEQ ID NO: 1607 - DNA, 24 bp, Artificial Sequence
  source: /note="Description of Artificial Sequence: Syntheticprobe"
agaaaggccctgacagatagtaac

SEQ ID NO: 1608 - DNA, 25 bp, Artificial Sequence
  source: /note="Description of Artificial Sequence: Syntheticprobe"
aacagaaaacttataccacattggg

SEQ ID NO: 1609 - DNA, 24 bp, Artificial Sequence
  source: /note="Description of Artificial Sequence: Syntheticprobe"
agaaaggccctgacagatagtaac

SEQ ID NO: 1610 - DNA, 25 bp, Artificial Sequence
  source: /note="Description of Artificial Sequence: Syntheticprobe"
aacagaaaacttataccacattggg

SEQ ID NO: 1611 - DNA, 22 bp, Artificial Sequence
  source: /note="Description of Artificial Sequence: Syntheticprobe"
tcccaccatgattgatgtgggg

SEQ ID NO: 1612 - DNA, 28 bp, Artificial Sequence
  source: /note="Description of Artificial Sequence: Syntheticprobe"
cagtaataacagcattgattgttcttac

SEQ ID NO: 1613 - DNA, 22 bp, Artificial Sequence
  source: /note="Description of Artificial Sequence: Syntheticprobe"
tcccaccatgattgatgtgggg

SEQ ID NO: 1614 - DNA, 28 bp, Artificial Sequence
  source: /note="Description of Artificial Sequence: Syntheticprobe"
cagtaataacagcattgattgttcttac

SEQ ID NO: 1615 - DNA, 26 bp, Artificial Sequence
  source: /note="Description of Artificial Sequence: Syntheticprobe"
ttccttgtcagaatccattacctgta

SEQ ID NO: 1616 - DNA, 21 bp, Artificial Sequence
  source: /note="Description of Artificial Sequence: Syntheticprobe"
ttgctgagtcacgagaacacg

SEQ ID NO: 1617 - DNA, 26 bp, Artificial Sequence
  source: /note="Description of Artificial Sequence: Syntheticprobe"
ttccttgtcagaatccattacctgta

SEQ ID NO: 1618 - DNA, 21 bp, Artificial Sequence
  source: /note="Description of Artificial Sequence: Syntheticprobe"
ttgctgagtcacgagaacacg

SEQ ID NO: 1619 - DNA, 24 bp, Artificial Sequence
  source: /note="Description of Artificial Sequence: Syntheticprobe"
tgcatacatagaagatcacagtgg

SEQ ID NO: 1620 - DNA, 23 bp, Artificial Sequence
  source: /note="Description of Artificial Sequence: Syntheticprobe"
tgtaagagtttttaggacccact

SEQ ID NO: 1621 - DNA, 24 bp, Artificial Sequence
  source: /note="Description of Artificial Sequence: Syntheticprobe"
tgcatacatagaagatcacagtgg

SEQ ID NO: 1622 - DNA, 23 bp, Artificial Sequence
  source: /note="Description of Artificial Sequence: Syntheticprobe"
tgtaagagtttttaggacccact

SEQ ID NO: 1623 - DNA, 20 bp, Artificial Sequence
  source: /note="Description of Artificial Sequence: Syntheticprobe"
gtgcttgtccccaggggata

SEQ ID NO: 1624 - DNA, 21 bp, Artificial Sequence
  source: /note="Description of Artificial Sequence: Syntheticprobe"
ggtataggcacacaatggtga

SEQ ID NO: 1625 - DNA, 20 bp, Artificial Sequence
  source: /note="Description of Artificial Sequence: Syntheticprobe"
gtgcttgtccccaggggata

SEQ ID NO: 1626 - DNA, 22 bp, Artificial Sequence
  source: /note="Description of Artificial Sequence: Syntheticprimer"
gtcatccctattggcaggttac

SEQ ID NO: 1627 - DNA, 20 bp, Artificial Sequence
  source: /note="Description of Artificial Sequence: Syntheticprimer"
cagggccttccttgtatctc

SEQ ID NO: 1628 - DNA, 23 bp, Artificial Sequence
  source: /note="Description of Artificial Sequence: Syntheticprimer"
cctcagtcaagttcagagtcttc

SEQ ID NO: 1629 - DNA, 23 bp, Artificial Sequence
  source: /note="Description of Artificial Sequence: Syntheticprimer"
tccatgaacctcaaggactattg

SEQ ID NO: 1630 - DNA, 20 bp, Artificial Sequence
  source: /note="Description of Artificial Sequence: Syntheticprimer"
ggtgattggtcctctgattg

SEQ ID NO: 1631 - DNA, 20 bp, Artificial Sequence
  source: /note="Description of Artificial Sequence: Syntheticprimer"
gactaacggctaacctggac

SEQ ID NO: 1632 - DNA, 20 bp, Artificial Sequence
  source: /note="Description of Artificial Sequence: Syntheticprimer"
tgagcctggccactatttac

SEQ ID NO: 1633 - DNA, 23 bp, Artificial Sequence
  source: /note="Description of Artificial Sequence: Syntheticprimer"
ctaggagagctggtaatttggtc

SEQ ID NO: 1634 - DNA, 21 bp, Artificial Sequence
  source: /note="Description of Artificial Sequence: Syntheticprimer"
actaccagcccaagatttctc

SEQ ID NO: 1635 - DNA, 22 bp, Artificial Sequence
  source: /note="Description of Artificial Sequence: Syntheticprimer"
cttcacaaagcggaagaatgtg

SEQ ID NO: 1636 - DNA, 19 bp, Artificial Sequence
  source: /note="Description of Artificial Sequence: Syntheticprimer"
tcgaagttccatcgctcac

SEQ ID NO: 1637 - DNA, 20 bp, Artificial Sequence
  source: /note="Description of Artificial Sequence: Syntheticprimer"
agcaaggcagtcagttacag

SEQ ID NO: 1638 - DNA, 21 bp, Artificial Sequence
  source: /note="Description of Artificial Sequence: Syntheticprimer"
cggatgtacagcgagtatgtg

SEQ ID NO: 1639 - DNA, 19 bp, Artificial Sequence
  source: /note="Description of Artificial Sequence: Syntheticprimer"
ccctgatttccacaagtcc

SEQ ID NO: 1640 - DNA, 24 bp, Artificial Sequence
  source: /note="Description of Artificial Sequence: Syntheticprimer"
aggccatctccttaatatttaccc

SEQ ID NO: 1641 - DNA, 22 bp, Artificial Sequence
  source: /note="Description of Artificial Sequence: Syntheticprimer"
cttccattctaggctacagctc

SEQ ID NO: 1642 - DNA, 21 bp, Artificial Sequence
  source: /note="Description of Artificial Sequence: Syntheticprimer"
tccattgccactgtgatcttc

SEQ ID NO: 1643 - DNA, 20 bp, Artificial Sequence
  source: /note="Description of Artificial Sequence: Syntheticprimer"
attccttgggctaccagttc

SEQ ID NO: 1644 - RNA, 22 nt, Artificial Sequence
  source: /note="Description of Artificial Sequence: Syntheticoligonucleotide"
uggagugugacaaugguguuug

SEQ ID NO: 1645 - DNA, 46 bp, Artificial Sequence
  source: /note="Description of Artificial Sequence: Syntheticchimeric probe"
  source: /note="Description of Combined DNA/RNA Molecule: Syntheticchimeric probe"
actggtaacccagacatgatcggtcaaacaccauugucacacucca

SEQ ID NO: 1646 - DNA, 20 bp, Artificial Sequence
  source: /note="Description of Artificial Sequence: Syntheticoligonucleotide"
ggctaaatcgctccaccaag

SEQ ID NO: 1647 - DNA, 12 bp, Artificial Sequence
  source: /note="Description of Artificial Sequence: Syntheticprobe"
gacatgatcggt

SEQ ID NO: 1648 - DNA, 24 bp, Artificial Sequence
  source: /note="Description of Artificial Sequence: Syntheticprobe"
actggtaacccagacatgatcggt

SEQ ID NO: 1649 - DNA, 10 bp, Artificial Sequence
  source: /note="Description of Artificial Sequence: Syntheticoligonucleotide"
accgatcatg

SEQ ID NO: 1650 - DNA, 10 bp, Artificial Sequence
  source: /note="Description of Artificial Sequence: Syntheticoligonucleotide"
gtactagcca

SEQ ID NO: 1651 - DNA, 10 bp, Artificial Sequence
  source: /note="Description of Artificial Sequence: Syntheticoligonucleotide"
catgatcggt

SEQ ID NO: 1652 - DNA, 10 bp, Artificial Sequence
  source: /note="Description of Artificial Sequence: Syntheticoligonucleotide"
tggctagtac

SEQ ID NO: 1653 - DNA, 48 bp, Artificial Sequence
  source: /note="Description of Artificial Sequence: Syntheticprobe"
actggtaacccagacatgatcggtaggtgattttggtctagctacagt

SEQ ID NO: 1654 - DNA, 48 bp, Artificial Sequence
  source: /note="Description of Artificial Sequence: Syntheticprobe"
actccctgtgggtgagcttaatggaggtgattttggtctagctacaga

SEQ ID NO: 1655 - DNA, 21 bp, Artificial Sequence
  source: /note="Description of Artificial Sequence: Syntheticprobe"
gaaatctcgatggagtgggtc

SEQ ID NO: 1656 - DNA, 75 bp, Artificial Sequence
  source: /note="Description of Artificial Sequence: Syntheticoligonucleotide"
ttcaaactgatgggacccactccatcgagatttcactgtagctagaccaaaatcacctatttttactgtgaggtc

SEQ ID NO: 1657 - DNA, 75 bp, Artificial Sequence
  source: /note="Description of Artificial Sequence: Syntheticoligonucleotide"
ttcaaactgatgggacccactccatcgagatttctctgtagctagaccaaaatcacctatttttactgtgaggtc

SEQ ID NO: 1658 - DNA, 24 bp, Artificial Sequence
  source: /note="Description of Artificial Sequence: Syntheticprobe"
aggtgattttggtctagctacagw

SEQ ID NO: 1659 - DNA, 21 bp, Artificial Sequence
  source: /note="Description of Artificial Sequence: Syntheticprobe"
gaaatctcgatggagtgggtc

SEQ ID NO: 1660 - DNA, 46 bp, Artificial Sequence
  source: /note="Description of Artificial Sequence: Syntheticprobe"
catttggtcattggttcaagacgagacatactggatacagctggac

SEQ ID NO: 1661 - DNA, 46 bp, Artificial Sequence
  source: /note="Description of Artificial Sequence: Syntheticprobe"
ctggtccgttgtggtccttctaacgacatactggatacagctggaa

SEQ ID NO: 1662 - DNA, 22 bp, Artificial Sequence
  source: /note="Description of Artificial Sequence: Syntheticprobe"
aagaagagtacagtgccatgag

(19) United States
(12) Patent Application Publication
(10) Pub. No.: US 2023/0175043 A1
(43) Pub. Date: Jun. 8, 2023

(54) KITS FOR DETECTING ONE OR MORE TARGET NUCLEIC ACID ANALYTES IN A SAMPLE AND METHODS OF MAKING AND USING THE SAME

(71) Applicant: Meso Scale Technologies, LLC., Rockville, MD (US)

(72) Inventors: John KENTEN, Boyds, MD (US); Sudeep KUMAR, Basking Ridge, NJ (US); Galina NIKOLENKO, Damascus, MD (US); Laukik SARDESAI, Gaithersburg, MD (US); Alexander K. TUCKER-SCHWARTZ, Bethesda, MD (US); George SIGAL, Rockville, MD (US); Seth Beebe HARKINS, Bethesda, MD (US); Robert UMEK, Rockville, MD (US); Timothy J. BREAK, Clarksburg, MD (US); Laure MOLLER, San Diego, CA (US)

(21) Appl. No.: 17/607,731

(22) PCT Filed: Apr. 30, 2020
(86) PCT No.: PCT/US2020/030754

§ 371 Date: Oct. 29, 2021

Related U.S. Application Data
(60) Provisional application No. 62/963,415, filed on Jan. 20, 2020.
(60) Provisional application No. 62/866,512, filed on Jun. 25, 2019.
(60) Provisional application No. 62/843,153, filed on May 3, 2019.

(51) Int. Cl. C12Q 1/6825

(52) U.S. Cl. CPC C12Q 1/6825; C12Q 1/6837; C12Q 2600/16

(57) ABSTRACT

Oligonucleotides, methods and kits are provided for detecting, identifying or quantifying one or more target analytes in a sample as well as methods for immobilizing oligonucleotides onto a support surface.

### FIELD OF THE INVENTION

The present disclosure relates to kits for detecting one or more target analytes in a sample and methods of making and using the same.

### SEQUENCE LISTING

The instant application contains a Sequence Listing which has been submitted electronically in ASCII format and is hereby incorporated by reference in its entirety. Said ASCII copy, created on Apr. 17, 2020, is named 0076-0006WO1_SL.txt and is 431,364 bytes in size.

### BACKGROUND OF THE INVENTION

Single nucleotide polymorphism (SNP) refers to a single nucleotide variation in the genome of an organism in which there are two or more distinct nucleotide residues (alleles) that each appear in a significant portion (>1%) of the population. SNPs are the most frequent form of sequence variation among individuals and are involved in the etiology of many heritable diseases. Wang et al. (1998), Large-Scale Identification, Mapping, and Genotyping of Single-Nucleotide Polymorphisms in the Human Genome, Science, 280:1077-1082. There are an estimated 10 million SNPs in the human genome, which can occur in coding and noncoding regions. Kruglyak et al. (2001) Variation is the Spice of Life, Nat. Genet., 27:234-236. Many SNPs have no effect on cell function, but others have been associated with inherited traits, genetic diseases, age-associated diseases, and responses to drugs and environmental factors.

Genotyping assays are genetic tests that are used to detect the presence of a nucleotide sequence in a sample and can be used to detect the presence of SNPs or other sequence variations in a sample, including, but not limited to deletions and insertions, duplications, and translocations. High-density oligonucleotide arrays use hundreds of thousands of probes arrayed on a chip to allow for the simultaneous interrogation of many nucleotide sequences.

Because large scale analysis of nucleotide sequences in a sample is required to associate a sequence with a disease or susceptibility to a disease, to link a sequence to individual variability in drug response, or to perform population studies, there remains a need for kits for identifying nucleotide sequences in a sample.

### SUMMARY OF THE INVENTION

The present invention relates to kits for identifying, detecting or quantifying one or more target analytes in a sample and methods of making and using the same. In one aspect, the target analyte includes a nucleic acid sequence. In one aspect, the target analyte includes a polypeptide sequence. In one aspect, the method or kit includes one or more capture molecules that are or can be immobilized in discrete binding domains on a support surface. In one aspect, a set of two or more non-cross-reactive capture oligonucleotides is provided.

In one aspect, the set of capture oligonucleotides is a subset of a parent set of capture oligonucleotides and one or more capture oligonucleotides in the set includes a nucleotide sequence having at least 20, 21, 22, 23, 24, 25, 26, 27, 28, 29, 30, 31, 32, 33, 34, 35 or 36 consecutive nucleotides of one or more nucleotide sequences from the parent set, wherein the parent set of capture oligonucleotides is selected from:
(a) capture set 1, including capture oligonucleotides having a nucleotide sequence selected from SEQ ID Nos: 1-64;
(b) capture set 2, including capture oligonucleotides having a nucleotide sequence selected from SEQ ID Nos: 65-122;
(c) capture set 3, including capture oligonucleotides having a nucleotide sequence selected from SEQ ID Nos: 123-186;
(d) capture set 4, including capture oligonucleotides having a nucleotide sequence selected from SEQ ID Nos: 187-250;
(e) capture set 5, including capture oligonucleotides having a nucleotide sequence selected from SEQ ID Nos: 251-308;
(f) capture set 6, including capture oligonucleotides having a nucleotide sequence selected from SEQ ID Nos: 309-372;
(g) capture set 7, including capture oligonucleotides having a nucleotide sequence selected from SEQ ID Nos: 373-436;
(h) capture set 8, including capture oligonucleotides having a nucleotide sequence selected from SEQ ID Nos: 437-494;
(i) capture set 9, including capture oligonucleotides having a nucleotide sequence selected from SEQ ID Nos: 495-558;
(j) capture set 10, including capture oligonucleotides having a nucleotide sequence selected from SEQ ID Nos: 559-662;
(k) capture set 11, including capture oligonucleotides having a nucleotide sequence selected from SEQ ID Nos: 623-680; and
(1) capture set 12, including capture oligonucleotides having a nucleotide sequence selected from SEQ ID Nos: 681-744.

In another aspect, a set of two or more non-cross-reactive capture oligonucleotides is provided, wherein the set of capture oligonucleotides is a subset of a parent set of capture oligonucleotides and one or more capture oligonucleotides in the set includes a nucleotide sequence having at least 95%, 96%, 97%, 98%, 99% or 100% identity to one or more nucleotide sequences from the parent set, wherein the parent set of capture oligonucleotides is selected from:
(a) capture set 1, including capture oligonucleotides having a nucleotide sequence selected from SEQ ID Nos: 1-64;
(b) capture set 2, including capture oligonucleotides having a nucleotide sequence selected from SEQ ID Nos: 65-122;
(c) capture set 3, including capture oligonucleotides having a nucleotide sequence selected from SEQ ID Nos: 123-186;
(d) capture set 4, including capture oligonucleotides having a nucleotide sequence selected from SEQ ID Nos: 187-250;
(e) capture set 5, including capture oligonucleotides having a nucleotide sequence selected from SEQ ID Nos: 251-308;
(f) capture set 6, including capture oligonucleotides having a nucleotide sequence selected from SEQ ID Nos: 309-372;
(g) capture set 7, including capture oligonucleotides having a nucleotide sequence selected from SEQ ID Nos: 373-436;
(h) capture set 8, including capture oligonucleotides having a nucleotide sequence selected from SEQ ID Nos: 437-494;
(i) capture set 9, including capture oligonucleotides having a nucleotide sequence selected from SEQ ID Nos: 495-558;
(j) capture set 10, including capture oligonucleotides having a nucleotide sequence selected from SEQ ID Nos: 559-662;
(k) capture set 11, including capture oligonucleotides having a nucleotide sequence selected from SEQ ID Nos: 623-680; and
(1) capture set 12, including capture oligonucleotides having a nucleotide sequence selected from SEQ ID Nos: 681-744.

In another aspect, a set of two or more non-cross-reactive capture oligonucleotides is provided, wherein the set of capture oligonucleotides is a subset of a parent set of capture oligonucleotides, wherein the parent set of capture oligonucleotides is selected from:
(a) capture set 1, including capture oligonucleotides having a nucleotide sequence selected from SEQ ID Nos: 1-64;
(b) capture set 2, including capture oligonucleotides having a nucleotide sequence selected from SEQ ID Nos: 65-122;
(c) capture set 3, including capture oligonucleotides having a nucleotide sequence selected from SEQ ID Nos: 123-186;
(d) capture set 4, including capture oligonucleotides having a nucleotide sequence selected from SEQ ID Nos: 187-250;
(e) capture set 5, including capture oligonucleotides having a nucleotide sequence selected from SEQ ID Nos: 251-308;
(f) capture set 6, including capture oligonucleotides having a nucleotide sequence selected from SEQ ID Nos: 309-372;
(g) capture set 7, including capture oligonucleotides having a nucleotide sequence selected from SEQ ID Nos: 373-436;
(h) capture set 8, including capture oligonucleotides having a nucleotide sequence selected from SEQ ID Nos: 437-494;
(i) capture set 9, including capture oligonucleotides having a nucleotide sequence selected from SEQ ID Nos: 495-558;
(j) capture set 10, including capture oligonucleotides having a nucleotide sequence selected from SEQ ID Nos: 559-662;
(k) capture set 11, including capture oligonucleotides having a nucleotide sequence selected from SEQ ID Nos: 623-680; and
(1) capture set 12, including capture oligonucleotides having a nucleotide sequence selected from SEQ ID Nos: 681-744.

In one aspect, the set of two or more non-cross-reactive capture oligonucleotides includes one or more capture oligonucleotides selected from:
(a) capture oligonucleotides having at least 20, 21, 22, 23, 24, 25, 26, 27, 28, 29, 30, 31, 32, 33, 34, 35 or 36 consecutive nucleotides of a sequence selected from SEQ ID Nos: 1-64;
(b) capture oligonucleotides including a sequence having at least 95%, 96%, 97%, 98%, 99% or 100% identity to a sequence selected from SEQ ID Nos: 1-64;
(c) capture oligonucleotides having at least 20, 21, 22, 23, 24, 25, 26, 27, 28, 29, 30, 31, 32, 33, 34, 35 or 36 consecutive nucleotides of a sequence having at least 95%, 96%, 97%, 98%, 99% or 100% identity to as sequence selected from SEQ ID Nos: 1-64;
(d) capture oligonucleotides including a sequence selected from SEQ ID Nos: 1-64; and
(e) capture oligonucleotides selected from any of (a)-(d).

In one aspect, the set of two or more non-cross-reactive capture oligonucleotides includes one or more capture oligonucleotides selected from:
(a) capture oligonucleotides including a sequence having at least 20, 21, 22, 23, 24, 25, 26, 27, 28, 29, 30, 31, 32, 33, 34, 35 or 36 consecutive nucleotides of a sequence selected from SEQ ID Nos: 1-10;
(b) capture oligonucleotides including a sequence having at least 95%, 96%, 97%, 98%, 99% or 100% identity to a sequence selected from SEQ ID Nos: 1-10;
(c) capture oligonucleotides having at least 20, 21, 22, 23, 24, 25, 26, 27, 28, 29, 30, 31, 32, 33, 34, 35 or 36 consecutive nucleotides of a sequence having at least 95%, 96%, 97%, 98%, 99% or 100% identity to as sequence selected from SEQ ID Nos: 1-10;
(d) capture oligonucleotides including a sequence selected from SEQ ID Nos: 1-10; and
(e) capture oligonucleotides selected from any of (a)-(d).

In one aspect, a set of non-cross-reactive capture oligonucleotides immobilized in an array is provided, wherein the set of non-cross-reactive oligonucleotides meet one or more of the following requirements:
(a) GC content between about 40% and about 50%; (b) AG content between about 30 and about 70%;
(c) CT content between about 30% and about 70%;
(d) a maximum string of base repeats in a sequence of no more than three;
(e) no undesired oligonucleotide-oligonucleotide interactions with strings of more than 7 complementary base pair matches in a row;
(f) no undesired oligonucleotide-oligonucleotide interactions with a string of 18 consecutive bases or less where:
   (i) the terminal bases at each end are complementary matches; and
   (ii) the sum of the complementary base pair matches minus the sum of the mismatches is greater than 7;
(g) no strings of 20, 21, 22, 23, 24, 25, 26, 27, 28, 29, 30, 31, 32, 33, 34, 35, or 36 base pairs or longer that match a sequence or complement of a sequence or both in a genome or in nature;
(h) differences in the free energy of hybridization for the sequences with their complements is less than about 1 kCal/mol, about 2 kCal/mol, about 3 kCal/mol or about 4 kCal/mol;
(i) no predicted hairpin loops with 4 or more consecutive matches in the stem; and
(j) no predicted hairpin loops with 4 or more consecutive matches in the stem and loop sizes greater than 6 bases.

In one aspect, the set of two or more non-cross-reactive capture oligonucleotides includes one or more capture oligonucleotides having a reactive functional group. In one aspect, the reactive functional group is attached to the capture oligonucleotide through a linker. In one aspect, the reactive functional group includes a thiol group.

In one aspect, one or more of the oligonucleotides are immobilized on a surface through the reactive functional group. In one aspect, the surface includes an electrode surface. In one aspect, the electrode includes a carbon-based electrode. In one aspect, the electrode includes a carbon ink electrode.

In one aspect, one or more non-reactive capture oligonucleotides are at least 20 nucleotides long. In one aspect, one or more non-reactive capture oligonucleotides are at least 24 nucleotides long. In one aspect, one or more non-reactive capture oligonucleotides are at least 36 nucleotides long.

In one aspect, a kit including a set of two or more non-cross-reactive capture oligonucleotides is provided. In one aspect, the kit includes a set of at least 2, 3, 4, 5, 6, 7, 8, 9, 10, 11, 12, 13, 14, 15, 16, 17, 18, 19, 20, 21, 22, 23, 24 or 25 and up to 64 non-cross-reactive capture oligonucleotides. In one aspect, the kit includes at least 10 non-cross-reactive capture oligonucleotides.

In one aspect, a set of two or more non-cross-reactive oligonucleotide tags is provided, wherein the set of oligonucleotide tags is a subset of a parent set of oligonucleotide tags and one or more oligonucleotide tags in the set includes a nucleotide sequence having at least 20, 21, 22, 23 or 24 consecutive nucleotides of one or more nucleotide sequences from the parent set, wherein the parent set is selected from:
(a) tag set 1, including oligonucleotide tags having a nucleotide sequence selected from SEQ ID Nos: 745-808;
(b) tag set 2, including oligonucleotide tags having a nucleotide sequence selected from SEQ ID Nos: 809-866;
(c) tag set 3, including oligonucleotide tags having a nucleotide sequence selected from SEQ ID Nos: 867-930;
(d) tag set 4, including oligonucleotide tags having a nucleotide sequence selected from SEQ ID Nos: 931-994;
(e) tag set 5, including oligonucleotide tags having a nucleotide sequence selected from SEQ ID Nos: 995-1052;
(f) tag set 6, including oligonucleotide tags having a nucleotide sequence selected from SEQ ID Nos: 1053-1116;
(g) tag set 7, including oligonucleotide tags having a nucleotide sequence selected from SEQ ID Nos: 1117-1180;
(h) tag set 8, including oligonucleotide tags having a nucleotide sequence selected from SEQ ID Nos: 1181-1238;
(i) tag set 9, including oligonucleotide tags having a nucleotide sequence selected from SEQ ID Nos: 1239-1302;
(j) tag set 10, including oligonucleotide tags having a nucleotide sequence selected from SEQ ID Nos: 1303-1366;
(k) tag set 11, including oligonucleotide tags having a nucleotide sequence selected from SEQ ID Nos: 1367-1424; and
(l) tag set 12, including oligonucleotide tags having a nucleotide sequence selected from SEQ ID Nos: 1425-1488.

In one aspect, a set of two or more non-cross-reactive oligonucleotide tags is provided, wherein the set of oligonucleotide tags is a subset of a parent set of oligonucleotide tags and one or more oligonucleotide tag in the set includes a nucleotide sequence having at least 95%, 96%, 97%, 98%, 99% or 100% identity to one or more nucleotide sequences from the parent set, wherein the parent set is selected from:
(a) tag set 1, including oligonucleotide tags having a nucleotide sequence selected from SEQ ID Nos: 745-808;
(b) tag set 2, including oligonucleotide tags having a nucleotide sequence selected from SEQ ID Nos: 809-866;
(c) tag set 3, including oligonucleotide tags having a nucleotide sequence selected from SEQ ID Nos: 867-930;
(d) tag set 4, including oligonucleotide tags having a nucleotide sequence selected from SEQ ID Nos: 931-994;
(e) tag set 5, including oligonucleotide tags having a nucleotide sequence selected from SEQ ID Nos: 995-1052;
(f) tag set 6, including oligonucleotide tags having a nucleotide sequence selected from SEQ ID Nos: 1053-1116;
(g) tag set 7, including oligonucleotide tags having a nucleotide sequence selected from SEQ ID Nos: 1117-1180;
(h) tag set 8, including oligonucleotide tags having a nucleotide sequence selected from SEQ ID Nos: 1181-1238;
(i) tag set 9, including oligonucleotide tags having a nucleotide sequence selected from SEQ ID Nos: 1239-1302;
(j) tag set 10, including oligonucleotide tags having a nucleotide sequence selected from SEQ ID Nos: 1303-1366;
(k) tag set 11, including oligonucleotide tags having a nucleotide sequence selected from SEQ ID Nos: 1367-1424; and
(l) tag set 12, including oligonucleotide tags having a nucleotide sequence selected from SEQ ID Nos: 1425-1488.

In one aspect, a set of two or more non-cross-reactive oligonucleotide tags is provided, wherein the set of oligonucleotide tags is a subset of a parent set of oligonucleotide tags, wherein the parent set is selected from:
(a) tag set 1, including oligonucleotide tags having a nucleotide sequence selected from SEQ ID Nos: 745-808;
(b) tag set 2, including oligonucleotide tags having a nucleotide sequence selected from SEQ ID Nos: 809-866;
(c) tag set 3, including oligonucleotide tags having a nucleotide sequence selected from SEQ ID Nos: 867-930;
(d) tag set 4, including oligonucleotide tags having a nucleotide sequence selected from SEQ ID Nos: 931-994;
(e) tag set 5, including oligonucleotide tags having a nucleotide sequence selected from SEQ ID Nos: 995-1052;
(f) tag set 6, including oligonucleotide tags having a nucleotide sequence selected from SEQ ID Nos: 1053-1116;
(g) tag set 7, including oligonucleotide tags having a nucleotide sequence selected from SEQ ID Nos: 1117-1180;
(h) tag set 8, including oligonucleotide tags having a nucleotide sequence selected from SEQ ID Nos: 1181-1238;
(i) tag set 9, including oligonucleotide tags having a nucleotide sequence selected from SEQ ID Nos: 1239-1302;
(j) tag set 10, including oligonucleotide tags having a nucleotide sequence selected from SEQ ID Nos: 1303-1366;
(k) tag set 11, including oligonucleotide tags having a nucleotide sequence selected from SEQ ID Nos: 1367-1424; and
(l) tag set 12, including oligonucleotide tags having a nucleotide sequence selected from SEQ ID Nos: 1425-1488.

In one aspect, the set of two or more non-cross-reactive oligonucleotide tags includes one or more oligonucleotide tags selected from:
(a) oligonucleotide tags including a sequence having at least 20, 21, 22, 23, or 24 consecutive nucleotides of a sequence selected from SEQ ID Nos: 745-808;
(b) oligonucleotide tags including a sequence having at least 95%, 96%, 97%, 98%, 99% or 100% identity to a sequence selected from SEQ ID Nos: 745-808;
(c) oligonucleotide tags having at least 20, 21, 22, 23 or 24 consecutive nucleotides of a sequence having at least 95%, 96%, 97%, 98%, 99% or 100% identity to as sequence selected from SEQ ID Nos: 745-808;
(d) oligonucleotide tags including a sequence selected from SEQ ID Nos: 745-808; and
(e) oligonucleotide tags selected from any of (a)-(d).

In one aspect, the set of two or more non-cross-reactive oligonucleotide tags includes one or more oligonucleotide tags selected from:
(a) oligonucleotide tags including a sequence having at least 20, 21, 22, 23 or 24 consecutive nucleotides of a sequence selected from SEQ ID Nos: 745-754;
(b) oligonucleotide tags including a sequence having at least 95%, 96%, 97%, 98%, 99% or 100% identity to a sequence selected from SEQ ID Nos: 745-754;
(c) oligonucleotide tags having at least 20, 21, 22, 23 or 24 consecutive nucleotides of a sequence having at least 95%, 96%, 97%, 98%, 99% or 100% identity to as sequence selected from SEQ ID Nos: 745-754;
(d) oligonucleotide tags including a sequence selected from SEQ ID Nos: 745-754; and
(e) oligonucleotide tags selected from any of (a)-(d).

In one aspect, the set includes two or more non-cross-reactive oligonucleotide tags, wherein one or more oligonucleotide tag in the set hybridizes to a non-complementary capture oligonucleotide less than 0.05% relative to a complementary capture oligonucleotide.

In one aspect, one or more oligonucleotide tags in the set include a label. In one aspect, the label is attached to the oligonucleotide tag through a linker. In one aspect, the label is selected from a radioactive, fluorescent, chemiluminescent, electrochemiluminescent, light absorbing, light scattering, electrochemical, magnetic and enzymatic label. In one aspect, the label includes an electrochemiluminescent label. In one aspect, the label is selected from a radioactive, fluorescent, colorimetric, antigenic and enzymatic label. In one aspect, the label includes a biotin or a hapten. In one aspect, the label includes biotin, fluorescein or digoxigenin. In one aspect, the label includes an organometallic complex including a transition metal. In one aspect, the transition metal includes ruthenium. In one aspect, the label includes a MSD SULFO-TAG™ label. In one aspect, the label includes a primary binding reagent that is a binding partner of a secondary binding reagent. In one aspect, the secondary binding reagent includes biotin, streptavidin, avidin, or an antibody. In one aspect, the primary binding reagent includes an oligonucleotide and the secondary binding reagent includes an oligonucleotide that is complementary to the primary binding reagent.

In one aspect, a kit that includes a set of two or more non-cross-reactive oligonucleotide tags is provided. In one aspect, the kit includes a set of at least 2, 3, 4, 5, 6, 7, 8, 9, 10, 11, 12, 13, 14, 15, 16, 17, 18, 19, 20, 21, 22, 23, 24 or 25 and up to 64 non-cross-reactive oligonucleotide tags. In one aspect, the kit includes a set of at least 10 non-cross-reactive oligonucleotide tags.

In one aspect, a method of immobilizing an oligonucleotide on a carbon-based support surface is provided. In one aspect, the oligonucleotide includes a thiol group. In one aspect, the method includes:
(a) printing one or more liquid droplets including the oligonucleotide on the carbon-based support surface;
(b) allowing the liquid droplets to spread on the surface;
(c) drying the liquid droplets to form dried droplets;
(d) immobilizing the oligonucleotide to the carbon-based support surface through the thiol group; and
(e) washing the dried droplets with a wash solution including a thiol-containing compound to remove non-immobilized oligonucleotide.

In one aspect, a method of manufacturing a carbon-based support surface having one or more immobilized oligonucleotides is provided. In one aspect, the method includes:
(a) printing one or more liquid droplets comprising an oligonucleotide comprising a thiol group on the carbon-based support surface;
(b) allowing the liquid droplets to spread on the surface;
(c) drying the liquid droplets to form dried droplets;
(d) immobilizing the oligonucleotide to the carbon-based support surface through the thiol group; and
(e) washing the dried droplets with a wash solution comprising a thiol-containing compound to remove non-immobilized oligonucleotide.

In one aspect, the carbon-based support surface includes a carbon-based electrode. In one aspect, the oligonucleotide is covalently attached to the carbon-based support surface through the thiol group. In one aspect, the method includes printing an array of liquid droplets that include the capture oligonucleotides on a plurality of binding domains on the carbon-based support surface.

In one aspect, a multi-well plate is provided that includes:
(a) one or more wells with one or more carbon-based electrodes; and
(b) a set of one or more non-cross-reactive capture oligonucleotides selected from a parent set of non-cross-reactive capture oligonucleotides as described herein, wherein one or more non-cross-reactive capture oligonucleotides are immobilized on one or more carbon-based electrodes.

In one aspect, a method of manufacturing a multi-well plate is provided in which the method includes:
(a) printing one or more liquid droplets comprising an oligonucleotide comprising a thiol group on the carbon-based electrode;
(b) allowing the liquid droplets to spread on the surface;
(c) drying the liquid droplets to form dried droplets;
(d) immobilizing the oligonucleotide to the carbon-based electrode through the thiol group; and
(e) washing the dried droplets with a wash solution comprising a thiol-containing compound to remove non-immobilized oligonucleotide.

In one aspect, the carbon-based support surface includes a carbon-based electrode. In one aspect, the oligonucleotide is covalently attached to the carbon-based support surface through the thiol group. In one aspect, the method includes printing an array of liquid droplets including the capture oligonucleotides on a plurality of binding domains on the support surface.

In one aspect, a method of immobilizing an array of capture oligonucleotides on a surface of one or more carbon-based electrodes is provided. In one aspect, the method includes:
(a) printing an array of liquid droplets including one or more capture oligonucleotides on a plurality of binding domains on the surface of one or more carbon-based electrodes, wherein one or more capture oligonucleotides include a thiol group;
(b) allowing the liquid droplets to spread on the surface;
(c) drying the liquid droplets to form dried droplets;
(d) incubating the dried droplets for a sufficient amount time to immobilize one or more oligonucleotides to the surface of the carbon-based electrode through the thiol groups; and
(e) washing the dried droplets with a wash solution including a thiol-containing compound to remove excess non-immobilized oligonucleotides.

In one aspect, the capture oligonucleotides printed on one binding domain of the array have a different sequence than capture oligonucleotides printed on other binding domains in the array. In one aspect, each binding domain includes less than about 0.1%, 0.09%, 0.08%, 0.07%, 0.06%, 0.05%, 0.04%, 0.03%, 0.02%, or 0.01% contaminating capture oligonucleotide. In one aspect, each binding domain includes less than about 0.05% contaminating capture oligonucleotide.

In one aspect, the method includes printing the array on a plurality of carbon-based electrodes. In one aspect, one or more carbon-based electrodes include a plurality of binding domains. In one aspect, the carbon-based electrode includes a carbon ink electrode. In one aspect, one or more carbon-based electrodes are in a multi-well plate.

In one aspect, the liquid droplets include a surfactant. In one aspect, the thiol-containing compound is water-soluble and has a molecular weight of less than about 200 g/mol, 175 g/mol, 150 g/mol, or 125 g/mol. In one aspect, the thiol-containing compound is selected from cysteine, cysteamine, dithiothreitol, 3-mercaptoproprionate, 3-mercapto-1-propanesulfonic acid, and combinations thereof. In one aspect, the thiol-containing compound includes cysteine. In one aspect, the wash solution includes between about 5 mM and about 750 mM cysteine, between about 10 mM and about 500 mM cysteine, or between about 25 mM and about 75 mM cysteine. In one aspect, the wash solution includes a pH buffering component, a surfactant, or a combination thereof and has a pH between about 7 and 9. In one aspect, the pH buffering component includes Tris, the surfactant includes Triton X-100 and the pH is about 8.0.

In one aspect, the method includes packaging the carbon-based electrode in a desiccated package. In one aspect, the carbon-based electrode is packaged in the desiccated package before the wash step. In one aspect, the carbon-based electrode is packaged in the desiccated package after the wash step.

In one aspect, kit is provided that includes:
(a) one or more support surfaces; and
(b) a set of one or more non-cross-reactive capture oligonucleotides selected from a parent set of non-cross-reactive capture oligonucleotides, wherein one or more non-cross-reactive capture oligonucleotides are immobilized on one or more support surfaces.

In one aspect, one or more support surfaces provided in the kit include a carbon-based support surface. In one aspect, one or more support surfaces include a carbon-based electrode.

In one aspect, a kit is provided that includes:
(a) one or more carbon-based electrodes having one or more surfaces; and
(b) a set of one or more non-cross-reactive capture oligonucleotides selected from a parent set of non-cross-reactive capture oligonucleotides, wherein one or more non-cross-reactive capture oligonucleotides are immobilized on one or more surfaces of the carbon-based electrode.

In one aspect, the kit includes one or more carbon ink electrodes.

In one aspect, the kit includes one or more non-cross-reactive capture oligonucleotides immobilized on one or more support surfaces in an array. In one aspect, one or more non-cross-reactive capture oligonucleotides are immobilized in one or more binding domains. In one aspect, two or more non-cross-reactive capture oligonucleotides immobilized in two or more unique binding domains, wherein the sequence of capture oligonucleotides immobilized on each unique binding domain are the same.

In one aspect, the kit includes one or more of the following components:
(a) a labeled oligonucleotide probe including a sequence complementary to a target sequence in a nucleic acid of interest,
(b) one or more blocking probes,
(c) one or more nucleoside triphosphates,
(d) one or more labeled nucleoside triphosphates,
(e) one or more labeled dideoxy nucleoside triphosphates;
(f) one or more ligase, and
(g) one or more polymerase.

In one aspect, the kit includes a plurality of labeled oligonucleotide probes including a first sequence complementary to a target sequence in a nucleic acid of interest and an oligonucleotide tag having a sequence that is complementary to a sequence of a capture oligonucleotide.

In one aspect, a kit for detecting, identifying or quantifying one or more target nucleotide sequences in a sample is provided, wherein one or more target nucleotide sequences include a polymorphic nucleotide, the kit including at least one pair of oligonucleotide probes including:
(i) a targeting probe including a single stranded oligonucleotide tag that is complementary to at least a portion of a capture oligonucleotide immobilized on the support surface and a first nucleic acid sequence that is complementary to a first region of the target nucleotide sequence in the sample; and
(ii) a detecting probe including a label and a second nucleic acid sequence that is complementary to a second region of the target nucleotide sequence that is adjacent to the first region to which the first nucleic acid sequence of the targeting probe sequence is complementary, wherein the targeting or detecting probe includes a terminal 3′ or 5′ nucleotide situated over the polymorphic nucleotide of the target nucleotide sequence.

In one aspect, the targeting probe has a terminal 3′ nucleotide complementary to a region of the target nucleotide sequence adjacent to the region to which the 5′ terminal nucleotide of the detecting probe is complementary. In one aspect, the terminal 3′ nucleotide of the targeting probe is complementary to the polymorphic nucleotide of the target nucleotide sequence.

In one aspect, the kit includes first and second targeting probes that bind the target nucleotide sequence, wherein the first and second targeting probes differ only in the terminal 3′ nucleotide. In one aspect, the first targeting probe is complementary to a wild type sequence and the second targeting probe is complementary to a mutant sequence.

In one aspect, the kit includes a plurality of pairs of oligonucleotide probes for a plurality of target nucleotide sequence.

In one aspect, the kit includes a detecting probe with a label attached to a 3′ end.

In one aspect, a kit is provided for detecting, identifying or quantifying one or more target nucleotide sequences in a sample, wherein one or more target nucleotide sequences include a polymorphic nucleotide and the kit includes one or more targeting probes including:
(a) a single stranded oligonucleotide tag that is complementary to at least a portion of a capture oligonucleotide immobilized on the support surface;
(b) a targeting nucleic acid sequence that is complementary to a target nucleotide sequence in the sample; and
(c) a label.

In one aspect, the kit includes one or more of the following:
(a) a polymerase; and
(b) one or more dideoxynucleotide triphosphates (ddNTPs).

In one aspect, the kit includes an oligonucleotide tag that is attached to a 5′ end of the targeting probe and the targeting nucleic acid sequence includes a 3′ end that is complementary to a nucleotide adjacent to a polymorphic nucleotide in one or more target nucleotide sequences in the sample. In one aspect, the oligonucleotide tag is attached to a 5′ end of the targeting probe and the targeting nucleic acid sequence includes a terminal 3′ nucleotide complementary to a polymorphic nucleotide of in one or more target nucleotide sequences in the sample.

In one aspect, the kit includes a plurality of probes including targeting nucleic acid sequences that are complementary to a plurality of target nucleotide sequences in the sample.

In one aspect, the kit includes labeled nucleoside triphosphate. In one aspect, the kit includes labeled nucleoside triphosphate and a secondary binding reagent. In one aspect, the kit includes the labeled nucleoside triphosphate includes a binding partner of a secondary binding reagent. In one aspect, the kit includes the secondary binding reagent includes avidin, streptavidin or an antibody and the labeled nucleoside triphosphate includes a biotin or hapten label.

In one aspect, the kit includes labeled nucleoside triphosphate with a radioactive, fluorescent, chemiluminescent, electrochemiluminescent, light absorbing, light scattering, electrochemical, magnetic or enzymatic label. In one aspect, the nucleoside triphosphate is labeled with an electrochemiluminescent label.

In one aspect, the kit includes labeled dideoxy nucleotide triphosphate complementary to the polymorphic nucleotide of the target nucleotide sequence.

In one aspect, the kit includes one or more of the following components:
(a) a hybridization buffer,
(b) a binding buffer,
(c) a label,
(d) a secondary binding reagent,
(d) a read buffer; and
(e) a unique kit identifier.

In one aspect, the hybridization buffer includes a nucleic acid denaturant. In one aspect, the nucleic acid denaturant includes formamide. In one aspect, the hybridization buffer is provided as two separate components that can be combined to form the hybridization buffer. In one aspect, the binding buffer includes a surfactant. In one aspect, the read buffer includes an electrochemiluminescence read buffer. In one aspect, the electrochemiluminescence read buffer includes one or more electrochemiluminescence co-reactants selected from a tertiary amine, tripropylamine, and N-butyldiethanolamine.

In one aspect, the kit includes one or more non-cross-reactive capture oligonucleotides immobilized in one or more binding domains on the surface of one or more carbon-based electrodes. In one aspect, one or more capture oligonucleotides include a thiol group and are covalently bound to the carbon-based surface through the thiol group. In one aspect, the capture oligonucleotide is attached to the thiol group through a linker. In one aspect, the capture oligonucleotide is covalently attached to the support surface. In one aspect, the non-cross-reactive capture oligonucleotides are immobilized in an array. In one aspect, the non-cross-reactive capture oligonucleotides are immobilized on a bead array.

In one aspect, the kit includes a set of two or more non-cross-reactive capture oligonucleotides selected from:
(a) capture oligonucleotides having at least 20, 21, 22, 23, 24, 25, 26, 27, 28, 29, 30, 31, 32, 33, 34, 35 or 36 consecutive nucleotides of a sequence selected from SEQ ID Nos: 1-64;
(b) capture oligonucleotides including a sequence having at least 95%, 96%, 97%, 98%, 99% or 100% identity to a sequence selected from SEQ ID Nos: 1-64;
(c) capture oligonucleotides including a sequence selected from SEQ ID Nos: 1-64; and
(d) capture oligonucleotides selected from any of the foregoing sets.

In one aspect, the kit includes a set of two or more non-cross-reactive capture oligonucleotides selected from:
(a) capture oligonucleotides including a sequence having at least 20, 21, 22, 23, 24, 25, 26, 27, 28, 29, 30, 31, 32, 33, 34, 35 or 36 consecutive nucleotides of a sequence selected from SEQ ID Nos: 1-10;
(b) capture oligonucleotides including a sequence having at least 95%, 96%, 97%, 98%, 99% or 100% identity to a sequence selected from SEQ ID Nos: 1-10;
(c) capture oligonucleotides including a sequence selected from SEQ ID Nos: 1-10; and
(d) capture oligonucleotides selected from any of the foregoing sets.

In one aspect, the oligonucleotide tag in the kit includes 24 or more nucleotides. In one aspect, the oligonucleotide tags include 36 or more nucleotides.

In one aspect, the oligonucleotide tag binds to a non-complementary capture oligonucleotide less than 0.05% relative to a complementary capture oligonucleotide.

In one aspect, the kit includes more than one electrode having one or more binding domains.

In one aspect, the kit includes two or more capture oligonucleotides immobilized in two or more unique binding domains, wherein the sequence of capture oligonucleotides immobilized on each unique binding domain are the same. In one aspect, the support surface includes at least 1, 2, 3, 4, 5, 6, 7, 8, 9, 10, 15, 20, or 25 distinct capture oligonucleotides immobilized in at least 1, 2, 3, 4, 5, 6, 7, 8, 9, 10, 15, 20, or 25 unique binding domains. In one aspect, one or more binding domains include at least some capture oligonucleotides that are not covalently bound to the electrode surface through a thiol group. In one aspect, one or more binding domains include more than 10%, 15%, 20%, 25%, 50% or 75% capture oligonucleotides that are not covalently bound to the support surface through a thiol group. In one aspect, the binding domains include less than about 0.1%, 0.09%, 0.08%, 0.07%, 0.06%, 0.05%, 0.04%, 0.03%, 0.02%, or 0.01% contaminating capture oligonucleotides.

In one aspect, the kit includes a support surface that includes a multi-well plate. In one aspect, one or more wells of the multi-well plate include one or more electrodes. In one aspect, one or more electrodes include a carbon-based electrode. In one aspect, one or more electrodes include carbon ink. In one aspect, one or more electrode in one or more wells of the multi-well plate include one or more binding domains. In one aspect, one or more capture oligonucleotides are immobilized on one or more binding domains on the one or more electrodes.

In one aspect, the kit includes a wash solution including a thiol-containing compound. In one aspect, the thiol-containing compound is water-soluble and has a molecular weight less than about 200 g/mol, 175 g/mol, 150 g/mol, or 125 g/mol. In one aspect, the thiol-containing compound is selected from cysteine, cysteamine, dithiothreitol, 3-mercaptoproprionate, 3-mercapto-1-propanesulfonic acid and combinations thereof. In one aspect, the thiol-containing compound includes cysteine. In one aspect, the thiol-containing compound includes a zwitterion. In one aspect, the wash solution includes an aqueous solution. In one aspect, the aqueous wash solution includes between about 5 mM and 750 mM cysteine, between about 10 mM and about 500 mM cysteine, or between about 25 mM and about 75 mM cysteine. In one aspect, the wash solution includes a pH buffering component, a surfactant, or a combination thereof. In one aspect, the pH buffer includes Tris and the surfactant includes Triton X-100. In one aspect, the wash solution has a pH between 7 and 9. In one aspect, the wash solution has a pH of about 8. In one aspect, the wash solution includes between about 15 mM and about 25 mM Tris, between about 0.05% and about 0.15% triton X-100, between about 5 mM and 750 mM cysteine and has a pH of about 8.0. In one aspect, the wash solution includes between about 15mM and about 25 mM Tris, between about 0.05% and about 0.15% triton X-100, between about 25 mM and about 75 mM cysteine and has a pH of about 8.0. In one aspect, the wash solution includes about 20 mM Tris, about 0.1% Triton X-100, about 50 mM cysteine and has a pH of about 8.0. In one aspect, one or more components of the wash solution are provided in dry form. In one aspect, the kit includes a liquid diluent for reconstituting the components of the wash solution.

In one aspect, the kit includes one or more blocking probes. In one aspect, the kit includes one or pairs of blocking probes, wherein each pair of blocking probes includes:
(a) a first blocking probe including a sequence identical to the sequence of the targeting probe, but without the single stranded oligonucleotide tag; and
(b) a second blocking probe having a sequence identical to the sequence of the detecting probe, but without the label.

In one aspect, the kit includes at least one pair of blocking probe for each pair of oligonucleotide probes.

In one aspect, the kit includes a label selected from a radioactive, fluorescent, chemiluminescent, electrochemiluminescent, light absorbing, light scattering, electrochemical, magnetic and an enzymatic label. In one aspect, the label includes an electrochemiluminescent label. In one aspect, the label includes an organometallic complex including a transition metal. In one aspect, the transition metal includes ruthenium. In one aspect, the label includes a MSD SULFO-TAG™ label. In one aspect, the label includes a binding partner of a secondary binding reagent. In one aspect, the secondary binding reagent includes biotin, streptavidin, avidin, or an antibody. In one aspect, the label includes a hapten selected from biotin, fluorescein and digoxigenin. In one aspect, the label includes a primary binding agent including a first oligonucleotide sequence and the secondary binding reagent includes a second oligonucleotide sequence that is complementary to the first oligonucleotide sequence of the primary binding agent.

In one aspect, a method is provided for identifying, detecting or quantifying a target analyte. In one aspect, the method includes providing an array having one or more carbon-based electrodes having one or more surfaces; and one or more non-cross-reactive capture oligonucleotides immobilized in one or more binding domains on one or more surfaces of the one or more carbon-based electrodes; contacting the array with a composition including one or more target analytes, wherein the one or more target analytes are linked to an oligonucleotide tag that is complementary to at least a portion of a capture oligonucleotide immobilized on the support surface and a label; incubating the composition of one or more target analytes under conditions in which the oligonucleotide tags hybridize to their complementary capture oligonucleotides to form a hybridization complex; and identifying, detecting or quantifying the target analyte based on the presence or absence of the label in an array location.

In one aspect, the method includes contacting the array with a composition that includes a plurality of target analytes, wherein each target analyte is linked to an oligonucleotide tag that is complementary to a different capture oligonucleotide and the target analyte can be identified, detected or quantified based on the binding of the oligonucleotide tag in an array location. In one aspect, less than about 0.05% of the oligonucleotide tags bind to a non-complementary capture oligonucleotide on the array as compared to their corresponding complementary capture oligonucleotide. In one aspect, the oligonucleotide tags include ate least 12, 24, or 36 nucleotides. In one aspect, one or more electrodes include carbon ink electrodes. In one aspect, one or more electrodes are included in a multi-well plate. In one aspect, each well of the multi-well plate includes an electrode. In one aspect, the electrode in each well of the multi-well plate includes an array of capture oligonucleotides immobilized in a plurality of binding domains. In one aspect, one or more target analytes are linked to the oligonucleotide tag through a binding partner. In one aspect, the binding partner includes an antibody that specifically binds to the target analyte. In one aspect, the binding partner includes an oligonucleotide sequence that is complementary to an oligonucleotide sequence of the target analyte. In one aspect, the oligonucleotide tag and the binding partner are different regions of a single oligonucleotide strand. In one aspect, one or more target analytes includes a nucleic acid sequence and the binding partner includes an oligonucleotide sequence complementary to a target sequence in a nucleic acid sequence of interest.

In one aspect, the method includes incubating the composition of one or more target analytes at a temperature of between about 27° C. and about 47° C., a formamide concentration between about 21% and about 41%, a salt concentration between about 300 mM and about 500 mM and a pH between about 7.5 and about 8.5 such that the oligonucleotide tags hybridize to their complementary capture oligonucleotides to form a hybridization complex. In one aspect, incubating includes a temperature of about 37° C., a formamide concentration of about 31%, a salt concentration of about 400 mM and a pH of 8.0.

In one aspect, one or more target analytes are labeled with a primary binding reagent. In one aspect, one or more target analytes are incubated with one or more pairs of oligonucleotide probes that include:
(a) a targeting probe including a single stranded oligonucleotide tag that is complementary to at least a portion of a capture oligonucleotide immobilized on the support surface and a first nucleic acid sequence that is complementary to a first region of the target nucleotide sequence in the sample; and
(b) a detecting probe including a label and a second nucleic acid sequence that is complementary to a second region of the target nucleotide sequence,
wherein incubating includes incubating under conditions in which the detecting probes bind to their corresponding target analytes.

In one aspect, the targeting probe and the detecting probe can bind concurrently to the target analyte to form a sandwich complex. In one aspect, the targeting and detecting probes have nucleotide sequences that are complementary to adjacent sequences of the target nucleotide sequence. In one aspect, the targeting probe has a nucleic acid sequence with a 3′ end that hybridizes to the target nucleotide sequence adjacent to a 5′ end of the detecting probe. In one aspect, the 3′ nucleotide of the targeting probe is complementary to a polymorphic nucleotide in the target nucleotide sequence.

In one aspect, the method includes incubating the hybridization complex in the presence of a nucleic acid ligase under conditions wherein the nucleic acid ligase ligates the targeting and detecting probes to form a reaction product. In one aspect, the method includes exposing the reaction product to denaturing conditions that dissociate the reaction product from the target nucleotide sequence. In one aspect, the method includes adding one or more blocking probes to the reaction product. In one aspect, the method includes incubating one or more target analytes with a targeting probe including:
(a) a single stranded oligonucleotide tag that is complementary to at least a portion of a capture oligonucleotide immobilized on the support surface;
(b) a targeting nucleic acid sequence that is complementary to a target nucleotide sequence in the sample; and
(c) a label.

In one aspect, the method includes incubating one or more target analytes with a targeting probe under conditions in which the targeting nucleic acid sequence of the targeting probe hybridizes to its complementary sequence on the target nucleotide sequence to form a hybridization complex. In one aspect, the method includes incubating the hybridization complex in the presence of a nucleic acid polymerase under conditions in which the polymerase extends the first probe sequence to form an extension sequence. In one aspect, the method includes incubating the polymerase with one or more labeled nucleoside triphosphates, wherein the extension sequence includes labeled nucleoside triphosphate.

In one aspect, the method includes incubating the polymerase with one or more unlabeled nucleoside triphosphates, wherein the extension sequence is longer than one nucleotide in length and includes unlabeled nucleoside triphosphate. In one aspect, the labeled nucleoside triphosphates include chain-terminating nucleoside triphosphates and the extension sequence is one nucleotide in length. In one aspect, the labeled nucleoside triphosphates include labeled dideoxynucleoside triphosphates. In one aspect, the first nucleic acid sequence of the targeting probe has a 3′ end that is complementary to a nucleotide adjacent to a polymorphic nucleotide in the nucleic acid of interest and the polymerase adds a nucleotide to the 3′ end of the targeting nucleic acid sequence.

In one aspect, method includes washing the array with a wash buffer after the incubating step. In one aspect, washing includes soaking the array in a wash buffer under high stringency conditions. In one aspect, the high stringency conditions include a temperature of between about 27° C. and about 47° C., a formamide concentration between about 21% and about 41%, a salt concentration between about 300 mM and about 500 mM and a pH between 7.5 and 8.5. In one aspect, the high stringency conditions include a temperature of about 37° C., a formamide concentration of about 31%, a salt concentration of about 400 mM and a pH of 8.0. In one aspect, the method includes soaking the array under high stringency conditions for at least 5, 10, 30 or 60 minutes. In another aspect, the high stringency condition includes a low salt condition, for example, a buffer with a salt concentration of less than about 40 mM, 20 mM, 15 mM, or 10 mM. In one aspect, the high stringency conditions include a low salt condition such as 0.1 XPBS at 37° C.

In one aspect, the label includes an electrochemiluminescent label and the method includes a step of generating an assay signal by contacting the electrodes with an electrochemiluminescence read buffer including an electrochemiluminescence co-reactant, and applying an electrical potential to the electrodes. In one aspect, the co-reactant is selected from a tertiary amine, tripropylamine, N-butyldiethanolamine, and combinations thereof. In one aspect, the method includes imaging the assay signal to determine the assay signal associated with each capture oligonucleotide.

### BRIEF DESCRIPTION OF THE FIGURES

FIG. 1A is a schematic of an oligonucleotide ligation assay (OLA) hybridization step;

FIG. 1B is a schematic of an OLA ligation step; FIG. 1C is a schematic of an OLA detection step; FIG. 1D is a schematic of an OLA probe mismatch in which hybridization does not occur.

FIG. 2A is a schematic of a primer extension assay (PEA) in which a labeled ddNTP is added to the 3′ end of the probe; FIG. 2B is a schematic of a PEA in which an unlabeled ddNTP is added to the 3′ end of the probe.

FIG. 3 is a graph showing the effect of changing the length of a linker (or spacer) between a capture oligonucleotide and an electrode on hybridization of a probe to the capture oligonucleotide and detection using electrochemiluminescence.

FIG. 4 is a graph showing the effect of different capture oligonucleotide array wash conditions on the measured cross-reactivity of an oligonucleotide probe specific for one element of the array.

FIG. 5 is a graph comparing the assay signal for an electrochemiluminescence OLA for a BRAF mutation as a function of the concentration of nucleic acid template containing the target BRAF gene region and compares the signal generated with the mutant sequence vs. the wild type sequence.

FIG. 6 is a graph showing the assay signals generated by a panel of electrochemiluminescence OLAs as function of the concentration of their specific target sequences.

FIG. 7 is a graph showing that bridging background signals for a panel of electrochemiluminescence OLAs can be reduced by the inclusion of blocking oligonucleotides.

FIG. 8 is a graph showing that elevated background in an electrochemiluminescence OLA due to non-specific binding of a probe to a capture oligonucleotide can be reduced by including blocking oligonucleotides or the additional of a high stringency hot soak step.

FIG. 9 shows the predicted percentage of mutant BRAF and NRAS sequences vs. the actual percentage of mutant sequences for electrochemiluminescence OLA results from PCR-amplified genomic DNA extracted from mixtures of mutant and wildtype cells.

FIG. 10 shows the assays signals for an electrochemiluminescence PEA for the BRAF 1799T>A mutation as a function of the concentration of template nucleic acids representing the mutant and wildtype sequences, showing that the assay is specific for the mutant sequence.

FIG. 11 is a graph showing that a panel of electrochemiluminescence PEAs for BRAF and NRAS SNP had linear responses to input DNA concentration.

FIG. 12 shows the predicted percentage of mutant BRAF 1799T>A sequence vs. the actual percentage of mutant sequence using an electrochemiluminescence BRAF 1799T>A OLA assay to measure PCR-amplified genomic DNA extracted from mixtures of mutant and wildtype cells.

FIG. 13 shows the predicted percentage of mutant NRAS 181C>A sequence vs. the actual percentage of mutant sequence using an electrochemiluminescence NRAS 181C>A OLA assay to measure PCR-amplified genomic DNA extracted from mixtures of mutant and wildtype cells.

FIG. 14 shows the predicted percentage of mutant 182A>T sequence vs. the actual percentage of mutant sequence using an electrochemiluminescence 182A>T OLA assay to measure PCR-amplified genomic DNA extracted from mixtures of mutant and wildtype cells.

FIG. 15 shows an oligonucleotide ligation amplification (OLA) assay for detection, identification, and/or quantification of a target nucleotide sequence, e.g., a therapeutic oligonucleotide, in a sample that may contain oligonucleotide metabolites, as described in embodiments herein.

FIG. 16 shows a direct hybridization method for detection, identification, and/or quantification of a target nucleotide sequence, e.g., a therapeutic oligonucleotide, in a sample that may contain oligonucleotide metabolites, as described in embodiments herein.

FIG. 17 shows a nuclease protection assay (NPA) with direct surface coating for detection, identification, and/or quantification of a target nucleotide sequence, e.g., a therapeutic oligonucleotide, in a sample that may contain oligonucleotide metabolites, as described in embodiments herein.

FIG. 18 shows a hybridization/protection assay for detection, identification, and/or quantification of a target nucleotide sequence, e.g., a therapeutic oligonucleotide, in a sample that may contain oligonucleotide metabolites, as described in embodiments herein.

FIG. 19 shows a sandwich assay for detection, identification, and/or quantification of an antibody, e.g., an anti-drug antibody (ADA), in a sample.

FIG. 20 shows as schematic of a targeting probe and detecting probe bridged by a positive control oligonucleotide that includes nucleotide sequences that are complementary to an ASO sequence.

FIG. 21 shows a modification of the sandwich assay shown in FIG. 19 for detection, identification, and/or quantification of an antibody, e.g., an anti-drug antibody (ADA), in a sample.

### DETAILED DESCRIPTION OF THE INVENTION

### A. Definitions

Unless otherwise defined, scientific and technical terms used herein shall have the meanings that are commonly understood by those of ordinary skill in the art. Further, unless otherwise required by context, singular terms shall include pluralities and plural terms shall include the singular, for example, “a” or “an”, include pluralities, e.g., “one or more” or “at least one” and the term “or” can mean “and/or”, unless explicitly indicated to refer only to alternatives or the alternatives are mutually exclusive. The terms “including,” “includes” and “included”, are not limiting. Ranges provided herein, of any type, include all values within a particular range described and values about an endpoint for a particular range.

As used herein, the term “about” is used to modify, for example, the quantity of an ingredient in a composition, concentration, volume, process temperature, process time, yield, flow rate, pressure, and ranges thereof, employed in describing the invention. The term “about” refers to variation in the numerical quantity that can occur, for example, through typical measuring and handling procedures used for making compounds, compositions, concentrates or formulations; through inadvertent error in these procedures; through differences in the manufacture, source, or purity of starting materials or ingredients used to carry out the methods, and other similar considerations. The term “about” also encompasses amounts that differ due to aging of a formulation with a particular initial concentration or mixture and amounts that differ due to mixing or processing a formulation with a particular initial concentration or mixture. Where modified by the term “about,” the claims appended hereto include such equivalents.

As used herein, ranges expressed using the word “between” are inclusive of the range endpoints. Thus, for example, a range of between 50° C. and 70° C. includes 50° C. to 70° C., i.e., it includes the endpoints of 50° C. and 70° C.

Generally, nomenclatures used in connection with, and techniques of, cell and tissue culture, molecular biology, and protein and oligo- or polynucleotide chemistry and hybridization described herein are those well-known and commonly used in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A “target analyte” can include any molecule of interest capable of being detected and analyzed by the methods and kits described herein and can include biological molecules such as nucleic acids, proteins, carbohydrates, sugars and lipids. In one aspect, the target analyte is a target nucleotide sequence. In another aspect, the target analyte is a protein. In one aspect, the target analyte is a DNA binding protein. The term “target analyte” can refer to the entire molecule of interest or a segment or portion of the molecule of interest. In one aspect, the target analyte includes modified molecules, for example, labeled, cleaved, or chemically or enzymatically treated versions of the molecule of interest.

A “target nucleotide sequence” can include any nucleotide sequence of interest including, but not limited to, sequences found in the DNA or RNA of prokaryotic or eukaryotic DNA organisms. These may include single or double stranded DNA, single or double stranded RNA, DNA/RNA hybrids, or DNA/RNA mosaics. The target nucleotide sequence can include an miRNA, a therapeutic RNA, an mRNA, an RNA virus, or a combination thereof. For double-stranded nucleotide sequences, a target nucleotide sequence can be identified in either strand. The target nucleotide sequence can require extraction, e.g., nuclear DNA or viral genomic DNA or RNA, or can be directly manipulated in a sample, e.g., cell free fetal DNA or cell free tumor DNA in serum or plasma or therapeutic oligonucleotides in circulation. The target nucleotide sequence can be directly isolated from a biological sample or can include amplified sequences from a biological sample. Amplification methods are known and include, but are not limited to, polymerase chain reaction (PCR), whole genome amplification (WGA), reverse transcription followed by the polymerase chain reaction (RT-PCR), strand displacement amplification (SDA), or rolling circle amplification (RCA). Polymerases suitable for the amplification methods herein include, e.g., Taq, Phi, Bst, and Vent-exo, e.g., for DNA amplification, and T7 RNA polymerase, e.g., for RNA amplification.

A target nucleotide sequence can be an oligonucleotide, e.g., a therapeutic oligonucleotide. A “therapeutic oligonucleotide” as used herein refers to an oligonucleotide capable of interacting with a biomolecule to provide a therapeutic effect. In one aspect, the therapeutic oligonucleotide is an antisense oligonucleotide (ASO). ASOs are capable of influencing RNA processing and/or modulating protein expression. An ASO is a single-stranded oligonucleotide that binds to single-stranded RNA to inactivate the RNA. ASOs are single stranded oligonucleotides that are typically from about 5, 10, 15, 20 or 25 nucleotides to about 30, 35, 40, 45 or 50 nucleotides in length. In one aspect, the ASO binds to messenger RNA (mRNA) for a gene, thereby inactivating the gene. In one aspect, the gene is a disease gene. Thus, the ASO can inactivate mRNA of a disease gene to prevent or ameliorate production of a particular disease-causing protein. In one aspect, the ASO includes DNA, RNA, or combination thereof. Therapeutic oligonucleotides and ASOs are further described in, e.g., Goodchild, Methods MolBiol 764:1-15 (2011); Smith et al., Ann Rev Pharmacol Toxicol 59:605-630 (2019); and Stein et al., Mol Ther 25(5):1069-1075 (2017).

In one aspect, the target analyte is an anti-drug antibody (ADA). As used herein, an “anti-drug antibody” or “ADA” is an antibody that is elicited in vivo in an organism against a biopharmaceutical product. The ADA can be elicited against biopharmaceuticals such as therapeutic polypeptides, including, but not limited to, proteins and antibodies and therapeutic oligonucleotides, including, but not limited to, antisense oligonucleotides (ASOs), short interfering RNAs, microRNAs, and synthetic guide strands for CRISPR/Cas. ADA can include any antibody isotype that is capable of binding to the biopharmaceutical product, referred to as binding antibodies, and can also include a subpopulation of the binding antibodies that are able to inhibit functional activity of the therapeutic product, referred to as neutralizing antibodies. Detection of ADA can be an important measure of immunogenicity, which can affect both safety and efficacy of biopharmaceutical products.

Target nucleotide sequences, such as therapeutic oligonucleotides, in a sample can degrade, i.e., shorten, over time, due to various factors such as presence of nucleases, temperature, pH, salt concentration, and the like. Degradation products of the target nucleotide sequence are also referred to as oligonucleotide metabolites. In one aspect, the oligonucleotide metabolite is shorter than the target nucleotide sequence by 1 or more nucleotides, 2 or more nucleotides, 3 or more nucleotides, 4 or more nucleotides, 5 or more nucleotides, 6 or more nucleotides, 7 or more nucleotides, 8 or more nucleotides, 9 or more nucleotides, 10 or more nucleotides, 15 or more nucleotides, or 20 or more nucleotides. In one aspect, the oligonucleotide metabolite is shorter than the target nucleotide sequence by about 1%, about 2%, about 3%, about 4%, about 5%, about 6%, about 7%, about 8%, about 9%, or about 10%. In one aspect, a sample of the present disclosure includes a target nucleotide sequence, e.g., a therapeutic oligonucleotide, and one or more oligonucleotide metabolites, e.g., therapeutic oligonucleotide metabolites.

In one aspect, the target nucleotide sequence is a therapeutic oligonucleotide. In certain aspects, degradation of therapeutic oligonucleotide in a sample is indicative of a pharmacodynamic response to the therapeutic oligonucleotide. Degraded or shortened therapeutic oligonucleotides, also referred to herein as therapeutic oligonucleotide metabolites, may lose therapeutic effectiveness. Methods of the present disclosure can be used to measure the amount of target nucleotide sequence, e.g., therapeutic oligonucleotide, relative to oligonucleotide metabolites, e.g., therapeutic oligonucleotide metabolites. In one aspect, a method of the present disclosure are used to determine the pharmacokinetic parameters of a target nucleotide sequence, e.g., therapeutic oligonucleotide. In one aspect, the pharmacokinetic parameters of a target nucleotide sequence, e.g., therapeutic oligonucleotide, is determined by measuring the rate and/or amount of degradation of the target nucleotide sequence, e.g., therapeutic oligonucleotide, in a biological environment, e.g., a patient. In one aspect, the pharmacokinetic parameter measured is clearance, volume distribution, plasma concentration, half-life, peak time, peak concentration, rate of availability, or combination thereof. Further discussion of the measurement and interpretation of pharmacokinetic parameters can be found in, e.g., Benet, Eur J Respir Dis Suppl 134:45-61 (1984) and Le et al., “Overview of Pharmacokinetics,” Merck Manual Professional Version, revised May 2019. An oligonucleotide metabolite present in a sample may also interfere with the detection, identification, and/or quantification of target nucleotide sequence in the sample. Thus, it may be desirable to remove oligonucleotide metabolites from the sample. Accordingly, methods of the present disclosure can also be used to reduce and/or remove oligonucleotide metabolites from a sample, e.g., in order to obtain a more accurate measurement of the amount of target nucleotide sequence.

“Polymerase chain reaction” or “PCR” refers to a technique used for amplifying a target nucleotide sequence which involves repeated cycles of three steps: (1) denaturation, in which double-stranded DNA templates are heated to separate the strands; (2) annealing, in which primers bind regions flanking the target DNA sequences; and (3) extension, in which DNA polymerase extends the 3′ end of each primer along the template strand. PCR can employ a heat stable DNA polymerase, such as Taq polymerase.

“Nucleotide” refers to a monomeric unit that includes a nitrogenous base, a five-carbon sugar (ribose or deoxyribose) and at least one phosphate group. Nucleotides include ribonucleoside triphosphates, such as, ATP, UTP, CTG, and GTP, found in RNA; deoxyribonucleoside triphosphates, most commonly dATP, dCTP, dGTP, dTTP, found in DNA; and dideoxyribonucleoside triphosphates (ddNTPs), which lack a 3′—OH necessary for polymerase mediated elongation, including, for example, as ddATP, ddCTP, ddGTP and ddTTP.

“Oligonucleotide” or “oligo” refers to a nucleic acid having a nucleotide sequence between about 5 and about 100, about 10 and about 50, or about 10 and about 25 nucleotides in length or at least about 10, 15, 20, 25, 30, 35, 40, 45 or 50 and up to about 50, 75 or 100 nucleotides in length. Oligonucleotides, including, but not limited to, probes, primers, tags or capture oligonucleotides described herein, can be prepared using known methods, including, for example, the phosphoramidite method described by Beaucage and Carruthers (1981) Deoxynucleoside phosphoramidites - a new class of key intermediates for deoxypolynucleotide synthesis. Tetrahedron Lett., 22(2):1859-1862 or the triester method according to Matteucci and Caruthers (1981) Synthesis of deoxynucleotides on a polymer support. J. Am. Chem. Soc., 103(11):3185-3191.

The nucleotides and nucleic acids of the disclosure, including, for example, those in target sequences or oligonucleotide reagents of the disclosure, may include structural analogs that include non-naturally occurring chemical structures that can also participate in hybridization reactions. In one example, a nucleotide or nucleic acid may include a chemical modification that links it to a label or provides a reactive functional group that can be linked to a label, for example, through the use of amine or thiol-modified nucleotide bases, phosphates or sugars. The term “reactive functional group” refers to an atom or associated group of atoms that can undergo a further chemical reaction, for example, to form a covalent bond with another functional group. Examples of reactive functional groups include, but are not limited to, amino, thiol, hydroxy, and carbonyl groups. In one aspect, the reactive functional group includes a thiol group. Labels that can be linked to nucleotides or nucleic acids through these chemical modifications include, but are not limited to, detectable moieties such as biotin, haptens, fluorophores, and electrochemiluminescent (ECL) labels

In another aspect, a nucleotide can be modified to prevent enzymatic or chemical extension of nucleic acid chains into which it is incorporated, for example, by replacing the ribose or deoxyribose group with dideoxyribose. In another example, the backbone components that link together the nucleotide bases (e.g., the sugar or phosphate groups) can be modified or replaced, for example, through the use of peptide nucleic acids (PNAs) or by the incorporation of ribose analogues such as those found in 2′-O-methyl-substituted RNA, locked nucleic acids, bridged nucleic acids and morpholino nucleic acids. These “backbone” analogues may be present in one, some or all of the backbone linkages in a nucleic acid or oligonucleotide and may provide certain advantages such as hybridization products with improved binding stability or stability of the linkages to nucleases. In another example of nucleotide and nucleic acid structural analogues, unnatural nucleotide bases may be included. The unnatural (also referred to as “non-canonical” base) may hybridize with a natural (canonical) base or it may hybridize with another unnatural base.

“Isolated” refers to a target analyte, for example, a polypeptide or protein, or an oligonucleotide or nucleic acid sequence that is substantially or essentially free from other sequences or components which normally accompany or interact with it in its naturally occurring environment. In one aspect, an isolated nucleotide sequence includes components or sequences not found with the nucleic acid sequence in its natural environment. The term “isolated” also includes non-naturally-occurring or recombinantly produced oligonucleotide or protein sequences since such non-naturally-occurring or recombinantly produced sequences are not found in nature. In particular, a non-naturally-occurring or recombinantly produced oligonucleotide may have immediately contiguous sequences that are not found naturally-occurring.

As used herein, the term “variant” refers to an polypeptide or oligonucleotide sequence that is at least 95%, 96%, 97%, 98%, 99% or 100% identical to a reference polypeptide or oligonucleotide sequence or that includes at least 20, 21, 22, 23, 24, 25, 26, 27, 28, 29, 30, 31, 32, 33, 34, 35 or 36 consecutive amino acids or nucleotides of the reference sequence.

The term “identical” means that two polynucleotide or two polypeptide sequences include identical nucleic acid bases or identical amino acid residues, respectively, at the same positions over a comparison window. The term “% sequence identity” can be determined by comparing two aligned sequences over a window of comparison, determining the number of positions at which the identical nucleic acid base or amino acid residue occurs in both sequences to yield the number of matched positions, dividing the number of matched positions by the total number of positions in the comparison window, and multiplying the result by 100 to yield the percentage of sequence identity. The comparison window can include a full-length sequence or may be a subpart of a larger sequence. Various methods and algorithms are known for determining the percent identity between two or sequences, including, but not limited MEGALIGN (DNASTAR, Inc. Madison, Wis.), FASTA, BLAST, or ENTREZ.

“Capture oligonucleotide” refers to an oligonucleotide reagent that can be immobilized on a support surface and is designed to hybridize to (and, therefore, capture on the surface) a complementary oligonucleotide. In one aspect, the capture oligonucleotide is a single stranded sequence that can selectively hybridize, for example, under stringent hybridization conditions, with a single stranded oligonucleotide tag present on a target reaction product. In one aspect, the target reaction product is generated by oligonucleotide ligation using a target nucleotide sequence as a template. In another aspect, the target reaction product is generated by primer extension using a target nucleotide sequence as a template. Capture oligonucleotides may be provided in solid form, e.g., lyophilized, in solution, or immobilized to a support surface, e.g., on particles (e.g., microparticles, beads) or in arrays. Two or more capture oligonucleotides may be provided together. Examples of two or more capture oligonucleotides provided together include parent sets or subsets (also referred to herein as sets) of capture oligonucleotides as described herein.

“Probe” or “Primer” refers to a reagent that includes an oligonucleotide sequence that is capable of hybridizing to a target nucleotide sequence. Probes can include a single stranded sequence that is complementary or substantially complementary to a portion of the target nucleotide sequence. The probe may also include a tag sequence (which may also be referred to herein as a directing sequence) that is complementary to a capture oligonucleotide. The sequence that is complementary to the target nucleotide sequence and the tag sequence may be present on the same nucleic acid strand within the probe, or they may be present on different strands within the probe, for example, the probe may include a first strand having a sequence complementary to the target sequence and a bridging sequence and a second strand having a tag sequence and a sequence complementary to the bridging sequence on the first strand, wherein the first and second strands are hybridized or can hybridize through the bridging sequences. Probes can be DNA or RNA and may contain modified nitrogenous bases analogs or which have been modified by labels or linkers suitable for attaching labels. Probes should be sufficiently long to allow hybridization of the probe to the target nucleotide sequence, typically between about 5 and about 100, about 10 and about 50, about 20 and about 30, or at least about 5, 6, 7, 8, 9, 10, 15, 20 or 25 and up to about 30, 35, 40, 45, 50, 75 or 100 nucleotides in length. Probes can be prepared by any suitable method known in the art, including chemical or enzymatic synthesis or by cleavage of larger nucleic acids using non-specific nucleic acid-cleaving chemicals or enzymes, or with site-specific restriction endonucleases. In some applications, a probe that is hybridized to a complementary region in a target sequence can prime extension of the probe by a polymerase, acting as a starting point for replication of adjacent single stranded regions on the target sequence.

“Linker” (also referred to herein as “spacer”) refers to one or more atoms that join one chemical moiety to another chemical moiety, for example, one or more atoms that join a reactive functional group or label to an oligonucleotide. The linker can be a nucleotide or non-nucleotide compound that includes one or more atoms, for example, from about 2, 3, 4, 5, 6, 7, 8, 9 or 10 atoms to about 11, 12, 13, 14, 15, 16, 17, 18, 19, or 20 atoms and can include atoms such as carbon, oxygen, sulfur, nitrogen and phosphorus and combinations thereof. Examples of linkers include low molecular weight groups such as amide, ester, carbonate and ether groups, as well as higher molecular weight linking groups such as polyethylene glycol (PEG) and alkyl chains. Thus, linkers may comprise one or more atoms, units, or molecules.

“Label” refers to a chemical group or moiety that has a detectable physical property or is capable of causing a chemical group or moiety to exhibit a detectable physical property, including, for example, an enzyme that catalyzes conversion of a substrate into a detectable product. A label can be detected by spectroscopic, photochemical, biochemical, immunochemical, electrical, optical, chemical, or other methods. Examples of labels include, but are not limited to, radioisotopes, enzymes, substrates, fluorescent molecules, chemiluminescent moieties, electrochemiluminescent moieties, magnetic particles, and bioluminescent moieties. In another aspect, the label is a compound that is a member of a binding pair, in which a first member of the binding pair (which can be referred to as a “primary binding reagent”) is attached to a substrate, for example, an oligonucleotide, and the other member of the binding pair (which can be referred to as a “secondary binding reagent”) has a detectable physical property. Non-limiting examples of binding pairs include biotin and streptavidin, or avidin; complementary oligonucleotides; hapten and hapten binding partner; and antibody/antigen binding pairs.

“Detection” refers to detecting, observing, or quantifying the presence of a substance, such as an oligonucleotide, based on the presence or absence of a label.

“Complementary” refers to nucleic acid molecules or a sequence of nucleic acid molecules that interact by the formation of hydrogen bonds, for example, according to the Watson-Crick base-pairing model. For example, hybridization can occur between two complementary DNA molecules (DNA-DNA hybridization), two RNA molecules (RNA-RNA hybridization), or between complementary DNA and RNA molecules (DNA-RNA hybridization). Hybridization can occur between a short nucleotide sequence that is complementary to a portion of a longer nucleotide sequence. Hybridization can occur between sequences that do not have 100% “sequence complementarity” (i.e., sequences where less than 100% of the nucleotides align based on a base-pairing model such as the Watson-Crick base-pairing model), although sequences having less sequence complementarity are less stable and less likely hybridize than sequences having greater sequence complementarity. In one aspect, the nucleotides of the complementary sequences have 100% sequence complementarity based on the Watson-Crick model. In another aspect, the nucleotides of the complementary sequences have at least about 90%, 95%, 96%, 97%, 98% or 99% sequence complementarity based on the Watson-Crick model.

Whether or not two complementary sequences hybridize can depend on the stringency of the hybridization conditions, which can vary depending on conditions such as temperature, solvent, ionic strength and other parameters. The stringency of the hybridization conditions can be selected to provide selective formation or maintenance of a desired hybridization product of two complementary nucleic acid sequences, in the presence of other potentially cross-reacting or interfering sequences. Stringent conditions are sequence-dependent - typically longer complementary sequences specifically hybridize at higher temperatures than shorter complementary sequences. Generally, stringent hybridization conditions are between about 5° C. to about 10° C. lower than the thermal melting point (Tₘ) (i.e., the temperature at which 50% of the sequences hybridize to a substantially complementary sequence) for a specific nucleotides sequence at a defined ionic strength, concentration of chemical denaturants, pH and concentration of the hybridization partners. Generally, nucleotide sequences having a higher percentage of G and C bases hybridize under more stringent conditions than nucleotide sequences having a lower percentage of G and C bases. Generally, stringency can be increased by increasing temperature, increasing pH, decreasing ionic strength, or increasing the concentration of chemical nucleic acid denaturants (such as formamide, dimethylformamide, dimethylsulfoxide, ethylene glycol, propylene glycol and ethylene carbonate). Stringent hybridization conditions typically include salt concentrations of less than about 1 M, 500 mM, or 200 mM; hybridization temperatures above about 20° C., 30° C., 40° C., 60° C. or 80° C.; and chemical denaturant concentrations above about 10%, 20%, 30% 40% or 50%. Because many factors can affect the stringency of hybridization, the combination of parameters may be more significant than the absolute value of any parameter alone.

The term “complement” or “complementary” refers to two oligonucleotides whose bases form complementary base pairs, base by base, for example, in which A pair with T or U and C pairs with G. Perfect complementarity or 100% complementarity refers to the situation in which each nucleotide of one oligonucleotides sequence or region can hydrogen bond with each nucleotide of a second oligonucleotide strand or region. “Substantial complementarity” refers to sequences that are partially complementary and are able to hybridize under stringent hybridization conditions. Substantially complementary sequences need not hybridize along their entire length.

“Corresponding” can be used to refer to the relationship between a capture oligonucleotide and an oligonucleotide tag, wherein the oligonucleotide tag is designed to specifically bind to a particular capture oligonucleotide sequence under stringent hybridization conditions. In one aspect, an oligonucleotide tag specifically binds to its corresponding capture molecule and does not bind or cross-react with other capture molecules under stringent conditions. In one aspect, an oligonucleotide tag specifically binds to its corresponding capture molecule and does not bind or cross-react with other capture molecules in an array under stringent conditions. In one aspect, the oligonucleotide tag is a single stranded oligonucleotide that has a sequence that is complementary to at least part of a sequence of its “corresponding” capture oligonucleotide. In one aspect, the nucleotides of the “corresponding” oligonucleotide tag and capture oligonucleotide sequences have 100% sequence complementarity based on the Watson-Crick model. In another aspect, the nucleotides of the corresponding sequences have at least about 90%, 95%, 96%, 97%, 98% or 99% sequence complementarity based on the Watson-Crick model.

A single stranded polynucleotide has “direction” or “directionality” because adjacent nucleotides are joined by a phosphodiester bond between their 3′ and 5′ carbons atoms, such that the terminal 5′ and 3′ carbons are exposed at either end of the polynucleotide, which can be referred to as the 5′- (phosphoryl) and 3′- (hydroxyl) ends of the molecule. An “inverse” oligonucleotide has the reverse sequence as a reference oligonucleotide when read in the 5′- to 3′- direction. For example, for a reference oligonucleotide sequence 5′-ACCGATCATG-3′ (SEQ ID NO: 1649), the “inverse” oligonucleotide sequence would be 5′-GTACTAGCCA-3′ (SEQ ID NO: 1650).

According to the rules defined by Watson-Crick base pairing and the antiparallel nature of the DNA-DNA, RNA-RNA, and RNA-DNA double helices, a complement of a sequence includes a string of bases that are (or substantially are) Watson-Crick partners of the bases in the original sequence but ordered from 3′ to 5′. An example of a complement of the sequence 5′-ACCGATCATG-3′ (SEQ ID NO: 1649) would be 5′-CATGATCGGT-3′ (SEQ ID NO: 1651). When the term inverse complement is used herein with respect to a sequence, it is used to refer to the complement of the reverse of the original sequence. An example of an inverse complement of the sequence 5′-ACCGATCATG-3′ (SEQ ID NO: 1649) would be 5′-TGGCTAGTAC-3′ (SEQ ID NO: 1652).

“Cross-react” or “cross-reactive” refers to the ability of an oligonucleotide sequence to hybridize to more than one other oligonucleotide sequence in a sample. In one aspect, the term “cross-react” refers to the ability of a first oligonucleotide sequence to hybridize to a second oligonucleotide sequence in a sample, wherein the second oligonucleotide sequence is not complementary or substantially complementary to the first oligonucleotide sequence. In one aspect, the term “cross-react” or “cross-reactive” refers to the ability of a capture oligonucleotide to hybridize to more than one oligonucleotide tag or more than one tagged target nucleotide sequence in a sample. In one aspect, the cross-reactive capture oligonucleotide hybridizes to one or more oligonucleotide tags in a sample under stringent capture hybridization conditions. In one aspect, stringent capture hybridization conditions include a temperature of between 27° C. and 47° C., a formamide concentration between 21% and 41%, a salt concentration between 300 mM and 500 mM and a pH between 7.5 and 8.5. In one aspect, stringent capture hybridization conditions include a temperature of about 37° C., a formamide concentration of about 31%, a salt concentration of about 400 mM and a pH of 8.0.

“Non-cross-reactive” or “non-cross-reacting” refers to a first oligonucleotide sequence that hybridizes only to a particular oligonucleotide sequence in a sample, for example, the ability of a first oligonucleotide sequence to hybridize only to its corresponding complementary sequence in a sample. In one aspect, the term “non-cross-reactive” refers to the ability of a capture oligonucleotide to hybridize only to one oligonucleotide tag in a sample that include more than one oligonucleotide tag or more than one tagged target nucleotide sequences. In one aspect, the non-cross-reactive oligonucleotide probe hybridizes only to one oligonucleotide tag in a sample under stringent hybridization conditions. In one aspect, non-cross-reactive means that the ratio at which the first oligonucleotide binds to a sequence other than its complementary sequence in a sample is less than 0.05% under stringent capture hybridization conditions.

“Ligase” refers to a class of enzymes which can join nucleotide sequences together by catalyzing the formation of a phosphodiester bond between a 3′ hydroxyl of one nucleotide sequence having a 5′ phosphate of a second nucleotide sequence. Ligases include, E. coli DNA ligase, T4 DNA ligase, T4 RNA ligase, T. aquaticus (Taq) ligase, T. Thermophilus DNA ligase (e.g., HiFi ligase), or Pyrococcus DNA ligase. In one aspect, the ligase is a thermostable ligase. “Ligation” refers to the process of joining two nucleotide sequences together by the formation of a phosphodiester bond between a 3′ hydroxyl of one nucleotide sequence and a 5′ phosphate of a second nucleotide sequence.

“Array” refers to one or more support surfaces having more than one spatially distinct (i.e., not overlapping) addressable locations, referred to herein as binding domains or array elements. In one aspect, each addressable location includes an assay reagent, including, for example, a capture molecule.

A “support surface” refers to a surface material onto which, various substances, for example, oligonucleotides or polypeptides can be immobilized. A “support surface” can be planar or non-planar. In one aspect, the support surface includes a flat surface. In one aspect, the support surface is a plate with a plurality of wells, i.e., a “multi-well plate.” Multi-well plates can include any number of wells of any size or shape, arranged in any pattern or configuration. In another aspect, the support surface has a curved surface. In one aspect, the support surface is provided by one or more particles, beads or microspheres. The terms particles, beads or microspheres can be used interchangeably unless otherwise indicated. In one aspect, the support surface includes color coded particles, beads or microspheres. In one aspect, the support surface includes an assay module, such as an assay plate, slide, cartridge, bead, or chip. In one aspect, the support surface includes assay flow cells or assay fluidics.

In one aspect, the support surface includes a plurality of addressable locations (which may be referred to as “spots”), for example, as is typical in “gene chip” devices. In another aspect, the array includes a plurality of support surfaces that each have one addressable location, as in “bead array” approaches where each bead in a suspension of beads represents an addressable location (which, for example, may be addressed using flow cytometric or microscopic detection techniques). In another aspect, the array includes a plurality of support surfaces that each have one or more, or two or more addressable locations per surface. The addressable locations on a support surface can be arranged in uniform rows and columns or can form other patterns. The number of addressable locations on the array can vary, for example from less than 10 to more than 50, 100, 200, 500, or 1000. “Multiplexing” refers to the simultaneous analysis of more than one assay target in a single assay.

“Carbon-based” refers to a material that contains elemental carbon (C) as a principal component. Examples of carbon-containing or carbon-based materials include, but are not limited to, carbon, carbon black, graphitic carbon, glassy carbon, carbon nanotubes, carbon fibrils, graphite, carbon fibers and mixtures thereof. Carbon-based materials can include elemental carbon, including, for example, graphite, carbon black or carbon nanotubes. In one aspect, carbon-based materials include conducting carbon-polymer composites, conducting polymers, or conducting particles dispersed in a matrix, for example, carbon inks, carbon pastes, or metal inks. Conducting carbon particles include, for example, carbon fibrils, carbon black, or graphitic carbon, dispersed in a matrix, for example, a polymer matrix such as ethylene vinyl acetate (EVA), polystyrene, polyethylene, polyvinyl alcohol, polyvinyl acetate, polyvinyl chloride or acrylonitrile butadiene styrene (ABS). Such polymer matrices can also include copolymers with more than one type of component monomer which may include monomers selected from vinyl acetate, ethylene, vinyl alcohol, vinyl chloride, acrylonitrile, butadiene, styrene or other monomers.

“Allele” refers to a genomic variant of a target nucleotide sequence, which, when translated may result in a functional or dysfunctional gene product. Two allelic forms may be referred to as a “wild type allele” and a “mutant” or “variant” allele. “Wild type” refers to a nucleotide sequence that is predominant in a population. “Mutant” or “variant” refer to a nucleotide sequence that is less frequent in the population. A mutant or variant nucleotide sequence may or may not have functional consequences.

“Polymorphism” or “polymorphic site” refers to one variant in a group of two or more nucleic acids. “Single nucleotide variant” (also sometime called “single nucleotide polymorphism”, “SNP”, or “single nucleotide alteration”) refers to a variant involving only a single nucleotide. A single nucleotide variant can involve a substitution of one nucleotide for another at a polymorphic site or a deletion of a nucleotide from, or an insertion of a nucleotide into, a reference nucleotide sequence. Single nucleotide variants can be common (e.g., present in at least 1% of a population) or rare (e.g., present in less than 1% of a population).

“Kit” refers to a set of components that are provided or gathered to be used together, for example, to create a composition, to manufacture a device, or to carry out a method. A kit can include one or more components. The components of a kit may be provided in one package or in multiple packages, each of which can contain one or more of the components. A listed component of a kit, may in turn, also be provided as a single physical part or as multiple parts to be combined for the kit use. For example, an instrument component of a kit may be provided fully assembled or as multiple instrument parts to be assembled prior to use. Similarly, a liquid reagent component of a kit may be provided as a complete liquid formulation in a container, as one or more dry reagents and one or more liquid diluents to be combined to provide the complete liquid formulation, or as two or more liquid solutions to be combined to provide the complete liquid formulation. As is known in the art, kit components for assays are often shipped and stored separately due to having different storage needs, e.g., storage temperatures of 4° C. versus -70° C.

### B. Overview

Described herein are kits for identifying, detecting or quantifying one or more target analytes in a sample and methods for making and using the same. In one aspect, the method or kit includes one or more capture molecules that are or can be immobilized in discrete binding domains on a support surface. In one aspect, the capture molecules are single stranded capture oligonucleotides with nucleotide sequences that are complementary to a nucleotide sequence of a single stranded oligonucleotide tag attached to a probe or reaction product. In one aspect, a probe that includes an oligonucleotide tag is associated with a target analyte to direct the target analyte to the capture molecule. In one aspect, a target analyte is associated with a first probe that includes an oligonucleotide tag and a second probe that includes a label. In one aspect, a reaction product is generated using a target nucleotide sequence as a template. In one aspect, the reaction product includes an oligonucleotide tag and a label. In one aspect, the method or kit includes one or more oligonucleotide tags. In one aspect, hybridization between the capture oligonucleotide and the complementary nucleotide sequence of a tag on the reaction product immobilizes the reaction product to a support surface so that the captured reaction product can then be identified, detected, or quantified based on the appended label.

In one aspect, a method of immobilizing one or more oligonucleotides on a support surface is provided. In one aspect, the method includes immobilizing one or more oligonucleotides that include a thiol reactive group on a support surface. In one aspect, one or more capture oligonucleotides are immobilized on a support surface in one or more binding domains. In one aspect, the method includes a step of washing the support surface with a thiol-containing wash solution (also referred to herein as a blocking solution or a blocker) to remove unbound oligonucleotide. In one aspect, each binding domain includes less than about 0.1%, 0.09%, 0.08%, 0.07%, 0.06%, 0.05%, 0.04%, 0.03%, 0.02%, or 0.01% contaminating capture oligonucleotide.

In one aspect, the methods and kits for identifying, detecting or quantifying one or more target analytes in a sample described herein provide increased sensitivity over conventional methods. In one aspect, the methods and kits of the present disclosure are capable of detecting nanomolar, suitably picomolar, or more suitably femtomolar concentrations of a target analyte in a sample. In one aspect, the methods and kits of the present disclosure are capable of detecting at least about 0.1 fM, 1 fM, 25 fM, 50 fM, 75 fM or 100 fM and up to about 500 fM, 1 pM, 10 pM, 100 pM, 500 pM or 1 nM, or about 0.1 fM to about 1 nM, about 1 fM to about 100 pM, about 10 fM to about 10 pM, about 50 fM to about 1 pM, or about 100 fM to about 500 fM of a target analyte in a sample. In one aspect, the methods and kits of the present disclosure are capable of detecting about 0.1 fM, about 1 fM, about 2.5 fM, about 5 fM, about 10 fM, about 25 fM, about 50 fM, about 100 fM, about 250 fM, about 500 fM, about 1 pM, about 2.5 pM, about 5 pM, about 10 pM, about 25 pM, about 50 pM, about 100 pM, or about 1 nM of a target analyte in a sample.

In a specific aspect, the methods and kits provided herein are capable of detecting femtomolar concentrations of a polynucleotide, e.g., a therapeutic oligonucleotide or an RNA such as mRNA, in a sample, which advantageously allows for identification, detection, and/or quantification without the need for amplifying the polynucleotide.

In one aspect, the methods and kits provided herein are capable of reducing the amount of time required for identifying, detecting or quantifying one or more target analytes in a sample compared with conventional methods. In one aspect, the methods and kits of the present disclosure are capable of identifying, detecting or quantifying one or more target analytes in a sample in about 1 hour to about 48 hours, about 1.5 hours to about 24 hours, about 2 hours to about 18 hours, about 2.5 hours to about 12 hours, about 3 hours to about 10 hours, about 3.5 hours to about 8 hours, about 4 hours to about 6 hours, or about 4.5 hours to about 5 hours. In one aspect, the methods and kits of the present disclosure are capable of identifying, detecting or quantifying one or more target analytes in a sample in less than about 48 hours, less than about 36 hours, less than about 24 hours, less than about 18 hours, less than about 12 hours, less than about 10 hours, less than about 9 hours, less than about 8 hours, less than about 7 hours, less than about 6 hours, less than about 5 hours, less than about 4 hours, less than about 3 hours, less than about 2 hours, or less than about 1 hour.

### C. Capture Molecule

In one aspect, the method or kit includes one or more capture molecules that are or can be immobilized in discrete binding domains on a support surface. In one aspect, the capture molecules are not naturally occurring sequences. In another aspect, the capture molecules are recombinantly produced. In one aspect, sequences for a set of non-cross-reactive capture molecules are generated using a mathematical algorithm.

In one aspect, the capture molecules are single stranded capture oligonucleotides having nucleotide sequences that are complementary to a nucleotide sequence of a single stranded oligonucleotide tag. In one aspect, the oligonucleotide tag is attached to a target analyte. In one aspect, the oligonucleotide tag is attached to a probe that is associated with a target analyte. In one aspect, the oligonucleotide tag is attached to a reaction product generated using a target nucleotide sequence as a template. In one aspect, hybridization between the capture oligonucleotide and the complementary nucleotide sequence of an oligonucleotide tag immobilizes the target of interest or reaction product to a support surface. The captured target or reaction product can then be identified, detected, or quantified based on an appended label.

In one aspect, the method or kit includes a distinct capture oligonucleotide for each target nucleotide sequence to be identified, detected or measured. In one aspect, hybridization between a plurality of capture oligonucleotides and their complementary oligonucleotide tags occurs simultaneously in parallel across an array of capture oligonucleotides. An array may comprise or consist of two or more capture oligonucleotides described herein. Thus, an array may comprise 2-150 or more capture oligonucleotides, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to 60, 65, 70, 75, 80, 85, 90, 95, 100, 110, 120, 130, 140, or 150 capture oligonucleotides. The oligonucleotides in an array may comprise or consist of a “parent set” or a “subset” (also referred to herein as “set”) of oligonucleotides as described herein.

In one aspect, one or more capture oligonucleotides include single stranded nucleic acid sequences, including for example, nucleic acid sequences including deoxyribonucleic acids (DNA), ribonucleic acids (RNA), or structural analogs that include non-naturally occurring chemical structures that can also participate in hybridization reactions.

In one aspect, the capture oligonucleotides used in a particular array have similar binding energies or melting temperatures (Tm), for example, within at least about 0.5° C., 1° C., 2° C., 3° C., 4° C., or 5° C. of each other, wherein the melting temperature (Tₘ) of an oligonucleotide refers to the temperature at which 50% of the oligonucleotides is hybridized with its complement and 50% is free in solution. Tₘ can be determined using known methods, for example, by measuring the absorbance change of the oligonucleotide with its complement as a function of temperature. In one aspect, the capture oligonucleotide has a melting temperature (Tₘ) at 50 mM NaCl of between about 50° C. and about 70° C., 55° C. and about 65° C., or at least about 50° C., 55° C., or 60° C. and up to about 60° C., 65° C., or 70° C. In one aspect, the capture oligonucleotide has a GC content between about 40% and about 60%, or about 40% and about 50%.

In one aspect, the capture oligonucleotide is between about 20 and about 100, about 30 and about 50, or about 35 and about 40 nucleotides in length, for example, at least about 20, 21, 22, 23, 24, 25, 26, 27, 28, 29, 30, 31, 32, 33, 34, 35, or 36 and up to about 36, 37, 38, 39, 40, 41, 42, 43, 44, 45, 46, 47, 48, 49, 50, 75 or 100 nucleotides in length. In one aspect, the capture oligonucleotide includes at least 20, 24, 30 or 36 nucleotides. Capture oligonucleotides that are at least about 20, 24, 30 or 36 nucleotides in length are able to bind to the tagged target or reaction product and remain bound at higher elevated temperatures and with improved specificity (i.e., less non-specific binding) as compared to shorter capture oligonucleotides. In one aspect, one or more capture oligonucleotides in an array are not identical in length to the nucleic acid sequence of its complementary oligonucleotide tag. In fact, it may be desirable to include a capture oligonucleotide with a sequence that is longer than its complementary single stranded oligonucleotide tag, for example, by up to 5, 10, 15, 20 or 25 bases. In one aspect, the tagged target or reaction product and capture oligonucleotide are included at about a 1:1 ratio. In another aspect, the tagged target or reaction product is present in excess to increase the likelihood of binding the tagged target or reaction product to the capture oligonucleotide. In one aspect, the tagged reaction product and capture oligonucleotide are included at about a 2:1, 3:1, 4:1 or 5:1 ratio.

In one aspect, one or more capture oligonucleotides are covalently or non-covalently immobilized to a support surface. In one aspect, one or more capture oligonucleotides are covalently or non-covalently immobilized to one or more binding domains on a support surface. In one aspect, the capture oligonucleotide is adsorbed to the support surface via electrostatic interactions, for example, between a negatively charged phosphate group on the oligonucleotide and a positive charge on the support surface. In one aspect, one or more capture oligonucleotides are immobilized to the support surface through the binding of a first binding partner attached (directly or through a linker moiety) to the capture oligonucleotide to a second binding partner that is immobilized on the surface. In one aspect, one or more capture oligonucleotides are covalently immobilized to the support surface. In one aspect, one or more capture oligonucleotides are directly immobilized to the support surface. In another aspect, the capture oligonucleotide is immobilized to the support surface through a linker.

In one aspect, one or more capture oligonucleotides include a reactive functional group. In one aspect, the functional group includes a thiol (—SH) or amine (—NH₂) group. In one aspect, one or more capture oligonucleotides are immobilized to the support surface through a reactive functional group. In one aspect, one or more capture oligonucleotides are immobilized to the support surface through a reactive functional group that is attached to the capture oligonucleotide through a linker. In one aspect, the capture oligonucleotide is immobilized to the support surface through a thiol or amine group. In one aspect, the capture oligonucleotide is immobilized to the support surface through a thiol or amine group that is attached to the capture oligonucleotide through a linker (also referred to herein as “spacer”). In one aspect, the linker includes between about 3 and about 20 atoms or molecules or units, or at least about 3, 4, 5, 6, 7, 8, 9, 10 and up to about 10, 11, 12, 13, 14, 15, 16, 17, 18, 19 or 20 atoms or molecules or units. In one aspect, the linker is a carbon atom linker. In one aspect, the linker is an ethylene glycol linker, or a polyethylene glycol (PEG) linker. In one aspect, the linker includes up to 3, 4, 5, or 6 successive PEG units. In another aspect, the linker includes three successive PEG units. In another aspect, the linker includes six successive PEG units. The linker may have the structure shown in Example 2.

In one aspect, one or more capture oligonucleotides are immobilized to a support surface that has been pretreated with a protein such as Bovine Serum Albumin (BSA). In another aspect, the capture oligonucleotide is immobilized to the support surface through a cross-linking agent. Suitable homo-bifunctional and hetero-bifunctional cross-linking agents for connecting proteins and nucleic acids to each other or to other materials are well known in the art, see for example, the Thermo Scientific Crosslinking Technical Handbook, published by Thermo Fisher Scientific, 2012). In one aspect the cross-linking agent is a hetero-bifunctional cross-linking agent comprising an amine reactive moiety (such as an N-hydroxysuccinimide or N-hydroxysulfosuccinimide ester) and a thiol-reactive moiety such as a maleimide, an iodosuccinimide or an activated disulfide (such as a pyridyldisulfide); such hetero-bifunctional cross-functional cross-linking agents include, for example, sulfosuccinimidyl-4-(N-maleimidomethyl)cyclohexane-1-carboxylate (Sulfo-SMCC). In one aspect, the amine reactive moiety (for example, the N-hydroxysuccinimide (NHS) moiety of SMCC) is reacted with a protein to introduce thiol-reactive moieties (for example, the maleimide moiety of SMCC) into the protein. The thiol-reactive moieties are, in turn, reacted with thiol-modified capture oligonucleotides to form protein-oligonucleotide conjugates that are linked through stable thioether bonds. Arrays of the protein-oligonucleotide conjugates can be formed by printing patterns of the reagents on surfaces that adsorb or react with proteins, to generate patterned arrays. In one aspect, arrays are formed by printing protein-oligonucleotide conjugates on graphitic carbon surfaces, for example, screen printed carbon ink electrodes. See, for example, U.S. Pat. Publication No. 2016/0069872, U.S. Pat. 6,977,722 and 7,842,246, the disclosures of which are hereby incorporated by reference in their entirety. In one aspect, one or more capture oligonucleotides are immobilized onto a support surface that has not been pretreated with a protein. In one aspect, the protein component of the protein-oligonucleotide used to immobilize oligonucleotides, as described above, is BSA.

In one approach, a computer algorithm is used to generate sets of capture oligonucleotides of a length discussed above (for example 24, 30 or 36-mers) that meet one or more of the following requirements: (a) GC content between about 40% and about 50%, (b) AG content between about 30 and about 70%, (c) CT content between about 30% and about 70%, (d) a maximum string of base repeats in a sequence of no more than three, (e) no undesired oligonucleotide-oligonucleotide interactions with strings of more than 7 complementary base pair matches in a row, (f) no undesired oligonucleotide-oligonucleotide interactions with a string of 18 consecutive bases or less where (i) the terminal bases at each end are complementary matches and (ii) the sum of the complementary base pair matches minus the sum of the mismatches is greater than 7, (g) no strings of 20 base pairs or longer (e.g., 20, 21, 22, 23, 24, 25, 26, 27, 28, 29, 30, 31, 32, 33, 34, 35, or 36 bp) that match a sequence (or complement of a sequence or both) in a given genome e.g., the human genome, or in sequences in nature, (h) differences in the free energy of hybridization for the sequences with their complements (or for the first 24 oligonucleotides from the 5′ end with its complement) less than about 1 kCal/mol, about 2 kCal/mol, about 3 kCal/mol or about 4 kCal/mol, (i) no predicted hairpin loops with 4 or more consecutive matches in the stem, (j) no predicted hairpin loops with 4 or more consecutive matches in the stem and loop sizes greater than 6 bases. In one aspect, at least criteria (a) through (h) are considered. An undesired oligonucleotide-oligonucleotide interaction in this context refers to an interaction of an oligonucleotide with itself, with another sequence within the set or with the complement of another sequence within the set. The free energy for hybridization (ΔG) is generally calculated for a specified ionic strength, temperature and pH, for example, physiological ionic strength and pH (about 150 mM NaCl, about pH 7.2) at room temperature (about 25° C.) or about 200 mM of a monovalent cation, about pH 7.0 at about 23° C., or another relevant condition. Alternatively or additionally, one or more of the following configurations can be avoided: formation of single nucleotide loops or single nucleotide mismatches positioned between G/C-rich sequences when paired with other capture oligonucleotides used in the assay.

In one aspect, the capture molecule includes an oligonucleotide sequence shown in any of SEQ ID NOs: 1-774 (Tables 1-12). In one aspect, the capture oligonucleotide has a nucleotide sequence that is at least 95%, 96%, 97%, 98%, or 99% identical to a sequence shown in any of SEQ ID NOs: 1-774. In another aspect, the capture oligonucleotide has a nucleotide sequence that includes at least 20, 21, 22, 23, 24, 25, 26, 27, 28, 29, 30, 31, 32, 33, 34, 35 or 36 consecutive nucleotides of a sequence shown in any of SEQ ID Nos: 1-774. In another aspect, the capture oligonucleotide has a nucleotide sequence that includes at least 20 consecutive nucleotides of a sequence shown in any of SEQ ID Nos: 1-774.

In another aspect, the capture oligonucleotide has a nucleotide sequence that is shown in any of SEQ ID Nos: 1-64. In one aspect, the capture oligonucleotide has a nucleotide sequence that is at least 95%, 96%, 97%, 98%, or 99% identical to a sequence shown in any of SEQ ID NOs: 1-64. In another aspect, the capture oligonucleotide has a nucleotide sequence that includes at least 20, 21, 22, 23, 24, 25, 26, 27, 28, 29, 30, 31, 32, 33, 34, 35 or 36 consecutive nucleotides of a sequence shown in any of SEQ ID Nos: 1-64. In another aspect, the capture oligonucleotide has a nucleotide sequence that includes at least 20 consecutive nucleotides of a sequence shown in any of SEQ ID Nos: 1-64.

In another aspect, the capture oligonucleotide has a nucleotide sequence that is shown in any of SEQ ID Nos: 1 to 10, 11 to 13, 25 to 26, 33 to 37, 42, 44 to 46, 54 and 59 to 62. In one aspect, the capture oligonucleotide has a nucleotide sequence that is at least 95%, 96%, 97%, 98%, or 99% identical to a sequence shown in any of SEQ ID NOs: 1 to 10, 11 to 13, 25 to 26, 33 to 37, 42, 44 to 46, 54 and 59 to 62. In another aspect, the capture oligonucleotide has a nucleotide sequence that includes at least 20, 21, 22, 23, 24, 25, 26, 27, 28, 29, 30, 31, 32, 33, 34, 35 or 36 consecutive nucleotides of a sequence shown in any of SEQ ID Nos: 1 to 10, 11 to 13, 25 to 26, 33 to 37, 42, 44 to 46, 54 and 59 to 62. In another aspect, the capture oligonucleotide has a nucleotide sequence that includes at least 20 consecutive nucleotides of a sequence shown in any of SEQ ID Nos: 1 to 10, 11 to 13, 25 to 26, 33 to 37, 42, 44 to 46, 54 and 59 to 62.

In another aspect, the capture oligonucleotide has a nucleotide sequence that is shown in any of SEQ ID Nos: 1-10. In one aspect, the capture oligonucleotide has a nucleotide sequence that is at least 95%, 96%, 97%, 98%, or 99% identical to a sequence shown in any of SEQ ID NOs: 1-10. In another aspect, the capture oligonucleotide has a nucleotide sequence that includes at least 20, 21, 22, 23, 24, 25, 26, 27, 28, 29, 30, 31, 32, 33, 34, 35 or 36 consecutive nucleotides of a sequence shown in any of SEQ ID Nos: 1-10. In another aspect, the capture oligonucleotide has a nucleotide sequence that includes at least 20 consecutive nucleotides of a sequence shown in any of SEQ ID Nos: 1-10. In another aspect, the capture oligonucleotide has a nucleotide sequence that is at least 95%, 96%, 97%, 98%, or 99% identical to a sequence that includes at least 20, 21, 22, 23, 24, 25, 26, 27, 28, 29, 30, 31, 32, 33, 34, 35 or 36 consecutive nucleotides of a sequence shown in any of SEQ ID Nos: 1-10. In another aspect, the capture oligonucleotide has a nucleotide sequence that is at least 95%, 96%, 97%, 98%, or 99% identical to a sequence that includes at least 20 consecutive nucleotides of a sequence shown in any of SEQ ID Nos: 1-10.

In one aspect, a base sequence is used to generate a set of non-cross-reactive capture oligonucleotides using an algorithm. In one aspect, up to four sets of non-cross-reactive capture oligonucleotides are generated: (a) a first set of non-cross-reactive capture oligonucleotides is generated using the base sequence; (b) a second set of non-cross-reactive capture oligonucleotides can be generated that have sequences that are complementary to the capture oligonucleotide sequences in the first set; (c) a third set of non-cross-reactive capture oligonucleotides can be generated that have the reverse sequence of the capture oligonucleotide sequences in the first set; and (d) a fourth set of non-cross-reactive capture oligonucleotides can be generated that have sequences that are the reverse-complement of the capture oligonucleotide sequences in the first set.

In one aspect, each set of non-cross-reactive capture oligonucleotides generated using the base sequence is referred to as a “parent set.” Two or more oligonucleotides from a parent set can be selected to form a “subset” (also referred to herein as “sets”) of non-cross-reactive capture oligonucleotides, wherein each oligonucleotide in the subset is a member of the same parent set (i.e., a subset cannot include capture oligonucleotides from more than one parent set).

For example, a base sequence can be used to generate: (a) a first parent set of non-cross-reactive capture oligonucleotides; (b) a second parent set of non-cross-reactive capture oligonucleotides can be generated that have sequences that are complementary to the capture oligonucleotide sequences in the first set; (c) a third parent set of non-cross-reactive capture oligonucleotides can be generated that have the reverse sequence of the capture oligonucleotide sequences in the first set; and (d) a fourth parent set of non-cross-reactive capture oligonucleotides can be generated that have sequences that are the reverse-complement of the capture oligonucleotide sequences in the first set.

A subset (or set) can include: (a) two or more non-cross-reactive capture oligonucleotides from the first parent set; (b) two or more non-cross-reactive capture oligonucleotides from the second parent set; (c) two or more non-cross-reactive capture oligonucleotides from the third parent set; or (d) two or more non-cross-reactive capture oligonucleotides from the fourth parent set. In one aspect, the set or subset of non-cross-reactive capture oligonucleotides includes between about 50 and about 150, about 50 and about 100, about 60 and about 75, or about 60 and about 65 non-cross-reactive capture oligonucleotides selected from a parent set of non-cross-reactive oligonucleotides. In one aspect, the set or subset of non-cross-reactive capture oligonucleotides includes at least about 2, 3, 4, 5, 6, 7, 8, 9, 10, 11, 12, 13, 14, 15, 16, 17, 18, 19, 20, 21, 22, 23, 24 or 25 and up to about 50, 51, 52, 53, 54, 55, 56, 57, 58, 59, 60, 61, 62, 63, 64, 65, 70, 75, 80, 85, 90, 95, 100, 125 or 150 non-cross-reactive oligonucleotides selected from a parent set of non-cross-reactive oligonucleotides.

In one aspect, a first base sequence is used to generate a first set of non-cross-reactive capture oligonucleotides shown in Table 1 (SEQ ID NOs: 1-64). The complementary sequences of this first set of non-cross-reactive capture oligonucleotides can be used to generate another set of non-cross-reactive sequences shown in Table 4 (SEQ ID NOs: 187-250). The reverse sequences of this first set of non-cross-reactive capture oligonucleotides can be used to generate another set of non-cross-reactive sequences shown in Table 7 (SEQ ID NOs: 373-436). The inverse complement sequences of this first set of non-cross-reactive capture oligonucleotides can be used to generate another set of non-cross-reactive sequences shown in Table 10 (SEQ ID NOs: 559-622).

In one aspect, the set of non-cross-reactive capture oligonucleotides includes two or more sequences from a parent set shown in Table 1 (SEQ ID NOs: 1-64). In one aspect, the set of non-cross-reactive capture oligonucleotides includes two or more sequences from a parent set shown in Table 4 (SEQ ID NOs: 187-250). In one aspect, the set of non-cross-reactive capture oligonucleotides includes two or more sequences from a parent set shown in Table 7 (SEQ ID NOs: 373-436). In one aspect, the set of non-cross-reactive capture oligonucleotides includes two or more sequences from a parent set shown in Table 10 (SEQ ID NOs: 559-622). In one aspect, the set of non-cross-reactive capture oligonucleotides is a subset of at least 2, 3, 4, 5, 6, 7, 8, 9, 10, 11, 12, 13, 14, 15, 16, 17, 18, 19, 20, 21, 22, 23, 24 or 25 and up to 64 non-cross-reactive sequences selected from a parent set shown in Table 1 (SEQ ID NOs: 1-64), Table 4 (SEQ ID NOs: 187-250), Table 7 (SEQ ID NOs: 373-436) or Table 10 (SEQ ID NOs: 559-622).

In one aspect, the set of non-cross-reactive capture oligonucleotides includes one or more capture oligonucleotides having a nucleotide sequence that is at least 95%, 96%, 97%, 98%, 99% or 100% identical to a sequence shown in Table 1 (SEQ ID NOs: 1-64), Table 4 (SEQ ID NOs: 187-250), Table 7 (SEQ ID NOs: 373-436) or Table 10 (SEQ ID NOs: 559-622). In another aspect, the set of non-cross-reactive capture oligonucleotides includes one or more capture oligonucleotides having a nucleotide sequence that includes at least 20, 21, 22, 23, 24, 25, 26, 27, 28, 29, 30, 31, 32, 33, 34, 35 or 36 consecutive nucleotides of a sequence shown in Table 1 (SEQ ID NOs: 1-64), Table 4 (SEQ ID NOs: 187-250), Table 7 (SEQ ID NOs: 373-436) or Table 10 (SEQ ID NOs: 559-622). In one aspect, the set of non-cross-reactive capture oligonucleotides includes one or more capture oligonucleotides having a nucleotide sequence that is at least 95%, 96%, 97%, 98%, 99% or 100% identical to a sequence that includes at least 20, 21, 22, 23, 24, 25, 26, 27, 28, 29, 30, 31, 32, 33, 34, 35 or 36 consecutive nucleotides of a sequence shown in Table 1 (SEQ ID NOs: 1-64), Table 4 (SEQ ID NOs: 187-250), Table 7 (SEQ ID NOs: 373-436) or Table 10 (SEQ ID NOs: 559-622).

In one aspect, a second base sequence is used to generate a second set of non-cross-reactive capture oligonucleotides shown in Table 2 (SEQ ID NOs: 65-122). The complementary sequences of this second set of non-cross-reactive capture oligonucleotides can be used to generate another set of non-cross-reactive sequences shown in Table 5 (SEQ ID NOs: 251-308). The reverse sequences of this second set of non-cross-reactive capture oligonucleotides can be used to generate another set of non-cross-reactive sequences shown in Table 8 (SEQ ID NOs: 437-494). The inverse complement sequences of this second set of non-cross-reactive capture oligonucleotides can be used to generate another set of non-cross-reactive sequences shown in Table 11 (SEQ ID NOs: 623-680).

In one aspect, the set of non-cross-reactive capture oligonucleotides includes two or more sequences from a parent set shown in Table 2 (SEQ ID NOs: 65-122). In one aspect, the set of non-cross-reactive capture oligonucleotides includes two or more sequences from a parent set shown in Table 5 (SEQ ID NOs: 251-308). In one aspect, the set of non-cross-reactive capture oligonucleotides includes two or more sequences from a parent set shown in Table 8 (SEQ ID NOs: 437-494). In one aspect, the set of non-cross-reactive capture oligonucleotides includes two or more sequences from a parent set shown in Table 11 (SEQ ID NOs: 623-680). In one aspect, the set of non-cross-reactive capture oligonucleotides is a subset of at least 2, 3, 4, 5, 6, 7, 8, 9, 10, 11, 12, 13, 14, 15, 16, 17, 18, 19, 20, 21, 22, 23, 24 or 25 and up to 64 non-cross-reactive sequences selected from a parent set shown in Table 2 (SEQ ID NOs: 65-122), Table 5 (SEQ ID NOs: 251-308), Table 8 (SEQ ID NOs: 437-494) or Table 11 (SEQ ID NOs: 623-680).

In one aspect, the set of non-cross-reactive capture oligonucleotides includes one or more capture oligonucleotides having a nucleotide sequence that is at least 95%, 96%, 97%, 98%, 99% or 100% identical to a sequence shown in Table 2 (SEQ ID NOs: 65-122), Table 5 (SEQ ID NOs: 251-308), Table 8 (SEQ ID NOs: 437-494) or Table 11 (SEQ ID NOs: 623-680).. In another aspect, the set of non-cross-reactive capture oligonucleotides includes one or more capture oligonucleotides having a nucleotide sequence that includes at least 20, 21, 22, 23, 24, 25, 26, 27, 28, 29, 30, 31, 32, 33, 34, 35 or 36 consecutive nucleotides of a sequence shown in Table 2 (SEQ ID NOs: 65-122), Table 5 (SEQ ID NOs: 251-308), Table 8 (SEQ ID NOs: 437-494) or Table 11 (SEQ ID NOs: 623-680). In one aspect, the set of non-cross-reactive capture oligonucleotides includes one or more capture oligonucleotides having a nucleotide sequence that includes at least 20, 21, 22, 23, 24, 25, 26, 27, 28, 29, 30, 31, 32, 33, 34, 35 or 36 consecutive nucleotides of that is at least 95%, 96%, 97%, 98%, 99% or 100% identical to a sequence shown in Table 2 (SEQ ID NOs: 65-122), Table 5 (SEQ ID NOs: 251-308), Table 8 (SEQ ID NOs: 437-494) or Table 11 (SEQ ID NOs: 623-680).

In one aspect, a third base sequence is used to generate a third set of non-cross-reactive capture oligonucleotides shown in Table 3 (SEQ ID NOs: 123-186). The complementary sequences of this third set of non-cross-reactive capture oligonucleotides can be used to generate another set of non-cross-reactive sequences shown in Table 6 (SEQ ID NOs: 309-372). The reverse sequences of this third set of non-cross-reactive capture oligonucleotides can be used to generate another set of non-cross-reactive sequences shown in Table 9 (SEQ ID NOs: 495-558). The inverse complement sequences of this third set of non-cross-reactive capture oligonucleotides can be used to generate another set of non-cross-reactive sequences shown in Table 12 (SEQ ID NOs: 681-744).

In one aspect, the set of non-cross-reactive capture oligonucleotides includes two or more sequences from a parent set shown in Table 3 (SEQ ID NOs: 123-186). In one aspect, the set of non-cross-reactive capture oligonucleotides includes two or more sequences from a parent set shown in Table 6 (SEQ ID NOs: 309-372). In one aspect, the set of non-cross-reactive capture oligonucleotides includes two or more sequences from a parent set shown in Table 9 (SEQ ID NOs: 495-558). In one aspect, the set of non-cross-reactive capture oligonucleotides includes two or more sequences from a parent set shown in Table 12 (SEQ ID NOs: 681-744). In one aspect, the set of non-cross-reactive capture oligonucleotides is a subset of at least 2, 3, 4, 5, 6, 7, 8, 9, 10, 11, 12, 13, 14, 15, 16, 17, 18, 19, 20, 21, 22, 23, 24 or 25 and up to 64 non-cross-reactive sequences selected from a parent set shown in Table 3 (SEQ ID NOs: 123-186), Table 6 (SEQ ID NOs: 309-372), Table 9 (SEQ ID NOs: 495-558) or Table 12 (SEQ ID NOs: 681-744).

In one aspect, the set of non-cross-reactive capture oligonucleotides includes one or more capture oligonucleotides having a nucleotide sequence that is at least 95%, 96%, 97%, 98%, 99% or 100% identical to a sequence shown in Table 3 (SEQ ID NOs: 123-186), Table 6 (SEQ ID NOs: 309-372), Table 9 (SEQ ID NOs: 495-558) or Table 12 (SEQ ID NOs: 681-744). In another aspect, the set of non-cross-reactive capture oligonucleotides includes one or more capture oligonucleotides having a nucleotide sequence that includes at least 20, 21, 22, 23, 24, 25, 26, 27, 28, 29, 30, 31, 32, 33, 34, 35 or 36 consecutive nucleotides of a sequence shown in Table 3 (SEQ ID NOs: 123-186), Table 6 (SEQ ID NOs: 309-372), Table 9 (SEQ ID NOs: 495-558) or Table 12 (SEQ ID NOs: 681-744). In one aspect, the set of non-cross-reactive capture oligonucleotides includes one or more capture oligonucleotides having a nucleotide sequence that includes at least 20, 21, 22, 23, 24, 25, 26, 27, 28, 29, 30, 31, 32, 33, 34, 35 or 36 consecutive nucleotides of that is at least 95%, 96%, 97%, 98%, 99% or 100% identical to a sequence shown in Table 3 (SEQ ID NOs: 123-186), Table 6 (SEQ ID NOs: 309-372), Table 9 (SEQ ID NOs: 495-558) or Table 12 (SEQ ID NOs: 681-744).

In one aspect, the set of non-cross-reactive capture oligonucleotides includes one or more capture oligonucleotides selected from: capture oligonucleotides having at least 20, 21, 22, 23, 24, 25, 26, 27, 28, 29, 30, 31, 32, 33, 34, 35 or 36 consecutive nucleotides of a sequence selected from SEQ ID Nos: 1-64; capture oligonucleotides having a sequence that is at least 95%, 96%, 97%, 98%, 99% or 100% identical to a sequence selected from SEQ ID Nos: 1-64; capture oligonucleotides having at least 20, 21, 22, 23, 24, 25, 26, 27, 28, 29, 30, 31, 32, 33, 34, 35 or 36 consecutive nucleotides of a sequence that is at least 95%, 96%, 97%, 98%, 99% or 100% identical to a sequence selected from SEQ ID Nos: 1-64; capture oligonucleotides having a sequence selected from SEQ ID Nos: 1-64; and combinations thereof.

In one aspect, the set of non-cross-reactive capture oligonucleotides includes one or more capture oligonucleotides selected from: capture oligonucleotides having at least 20, 21, 22, 23, 24, 25, 26, 27, 28, 29, 30, 31, 32, 33, 34, 35 or 36 consecutive nucleotides of a sequence selected from SEQ ID Nos: 1-10; capture oligonucleotides having a sequence that is at least 95%, 96%, 97%, 98%, 99% or 100% identical to a sequence selected from SEQ ID Nos: 1-10; capture oligonucleotides having at least 20, 21, 22, 23, 24, 25, 26, 27, 28, 29, 30, 31, 32, 33, 34, 35 or 36 consecutive nucleotides of a sequence that is at least 95%, 96%, 97%, 98%, 99% or 100% identical to a sequence selected from SEQ ID Nos: 1-10; capture oligonucleotides having a sequence selected from SEQ ID Nos: 1-10; and combinations thereof.

In one aspect, the capture oligonucleotide is covalently bound to a protein and immobilization on the support surface is achieved through adsorption of the protein to the support surface. Examples of proteins that may be used include an albumin, such as bovine serum albumin (BSA), an immunoglobulin or another protein selected for its ability to adsorb to the support surface. In another aspect, the capture oligonucleotide is attached (directly or through a linker) to a first binding partner from a binding partner pair and immobilization is achieved by binding of this first binding partner to a second binding partner from the binding partner pair that is immobilized on the support surface. Binding partner pairs that are suitable for use in immobilizing capture oligonucleotides include binding partner pairs know in the art such as biotin-streptavidin, biotin-avidin, antibody-hapten, antibody-epitope tag (for example, antibody-FLAG), nickel-NTA and receptor-ligand pairs. In one aspect, the capture oligonucleotide is covalently bound to the protein or the first binding partner through a thiol (-SH) or amine (-NH₂) group. This binding can be direct or through a linking group (for example, a bifunction linking group such as those described in the Thermo Scientific Crosslinking Technical Handbook, published by Thermo Fisher Scientific, 2012). In one aspect, the thiol or amine group is at the 5′- or 3′- end of the capture oligonucleotide. In one aspect, the capture oligonucleotide is a 5′-terminal thiolated oligonucleotide. In one aspect, the capture oligonucleotide is a 3′-terminal thiolated oligonucleotide. In one aspect, the thiol group is incorporated at an internal position of the capture oligonucleotide. In one aspect, the capture oligonucleotide has a nucleotide sequence that includes a sequence shown in any of SEQ ID NOs: 1489-1498 (Table 25). In one aspect, the capture oligonucleotide has a nucleotide sequence that is at least 95%, 96%, 97%, 98%, or 99% identical to a sequence shown in any of SEQ ID NOs: 1489-1498. In another aspect, the capture oligonucleotide has a nucleotide sequence that includes at least 20, 21, 22, 23, 24, 25, 26, 27, 28, 29, 30, 31, 32, 33, 34, 35 or 36 consecutive nucleotides of a sequence shown in SEQ ID Nos: 1489-1498. In another aspect, the capture oligonucleotide has a nucleotide sequence that includes at least 20, 21, 22, 23, 24, 25, 26, 27, 28, 29, 30, 31, 32, 33, 34, 35 or 36 consecutive nucleotides of a sequence that is at least 95%, 96%, 97%, 98%, or 99% identical to a sequence shown in any of SEQ ID NOs: 1489-1498.

In one aspect, the capture oligonucleotide is covalently bound to the support surface through a thiol (-SH) or amine (-NH₂) group. In one aspect, the thiol or amine group is at the 5′-or 3′- end of the capture oligonucleotide. In one aspect, the capture oligonucleotide is a 5′-terminal thiolated oligonucleotide. In one aspect, the capture oligonucleotide is a 3′-terminal thiolated oligonucleotide. In one aspect, the thiol group is incorporated at an internal position of the capture oligonucleotide. In one aspect, the capture oligonucleotide has a nucleotide sequence that includes a sequence shown in any of SEQ ID NOs: 1489-1498 (Table 25). In one aspect, the capture oligonucleotide has a nucleotide sequence that is at least 95%, 96%, 97%, 98%, or 99% identical to a sequence shown in any of SEQ ID NOs: 1489-1498. In another aspect, the capture oligonucleotide has a nucleotide sequence that includes at least 20, 21, 22, 23, 24, 25, 26, 27, 28, 29, 30, 31, 32, 33, 34, 35 or 36 consecutive nucleotides of a sequence shown in SEQ ID Nos: 1489-1498. In another aspect, the capture oligonucleotide has a nucleotide sequence that includes at least 20, 21, 22, 23, 24, 25, 26, 27, 28, 29, 30, 31, 32, 33, 34, 35 or 36 consecutive nucleotides of a sequence that is at least 95%, 96%, 97%, 98%, or 99% identical to a sequence shown in any of SEQ ID NOs: 1489-1498.

In one aspect, the capture oligonucleotide has a nucleotide sequence that is the complement, the reverse or the inverse complement of a nucleotide sequence shown in SEQ ID NOs: 1489-1498. In one aspect, the capture oligonucleotide has a nucleotide sequence that is the complement, the reverse or the inverse complement of a nucleotide sequence that is at least 95%, 96%, 97%, 98%, or 99% identical to a sequence shown in SEQ ID NOs: 1489-1498. In another aspect, the capture oligonucleotide has a sequence that is the complement, the reverse or the inverse complement of a sequence having at least 20, 21, 22, 23, 24, 25, 26, 27, 28, 29, 30, 31, 32, 33, 34, 35 or 36 consecutive nucleotides of a sequence shown in SEQ ID Nos: 1489-1498. In another aspect, the capture oligonucleotide has a nucleotide sequence that is the complement, the reverse or the inverse complement of a sequence that includes at least 20, 21, 22, 23, 24, 25, 26, 27, 28, 29, 30, 31, 32, 33, 34, 35 or 36 consecutive nucleotides of a sequence that is at least 95%, 96%, 97%, 98%, or 99% identical to a sequence shown in any of SEQ ID NOs: 1489-1498.

In one aspect, one or more capture oligonucleotides include only three bases (TAG) to reduce hybridization with native sequences, similar to Luminex x-TAG technology.

### D. Support Surface

In one aspect, one or more capture oligonucleotides are immobilized on a support surface. The capture oligonucleotides can be immobilized on a variety of support surfaces, including support surfaces used in conventional binding assays. In one aspect, the support surface has a flat surface. In another aspect, the support surface has a curved surface. In one aspect, the support surface includes an assay module, such as an assay plate, slide, cartridge, bead, or chip. In one aspect, the support surface includes color coded microspheres. See, for example, Yang et al. (2001) BADGE, BeadsArray for the Detection of Gene Expression, a High-Throughput Diagnostic Bioassay. Genome Res. 11(11): 1888-1898. In one aspect, the support surface includes one or more beads on which one or more capture oligonucleotides are immobilized.

Support surfaces can be made from a variety of suitable materials including polymers, such as polystyrene and polypropylene, ceramics, glass, composite materials, including, for example, carbon-polymer composites such as carbon-based inks. In one aspect, the support surface is a carbon-based support surface.

In one aspect, the support surface is provided by one or more particles or “beads”. In one aspect, the beads can have a diameter up to about 1 cm (or 10,000 µm), 5,000 µm, 1,000 µm, 500 µm or 100 µm. In one aspect, beads have a diameter between about 10 nm and about 100 µm, between about 100 nm and about 10 µm or between about 0.5 µm and about 5 µm. In one aspect, the beads are paramagnetic, providing the ability to capture the beads through the use of a magnetic field. In one aspect, the support surface is provided by streptavidin or avidin-coated magnetic beads and biotin-labeled capture oligonucleotides are immobilized on the beads.

In one aspect, the support surface is a plate with a plurality of wells, i.e., a “multi-well plate.” Multi-well plates can include any number of wells of any size or shape, arranged in any pattern or configuration. In one aspect, the multi-well plate includes between about 1 to about 10,000 wells. In one aspect, the multi-well assay plates use industry standard formats for the number, size, shape and configuration of the plate and wells. Examples of standard formats include 96-, 384-, 1536- and 9600-well plates, with the wells configured in two-dimensional arrays. Other multi-well formats include single well, two well, six well and twenty-four well and 6144 well plates. In one aspect, the support surface includes a 96 well-plate.

In one aspect, the support surface includes a two-dimensional patterned array in which capture molecules are printed at known locations, referred to as binding domains. In one aspect, the support surface includes a patterned array of discrete, non-overlapping, addressable binding domains to which capture oligonucleotides are immobilized, wherein the sequence of the capture oligonucleotide in each binding domain is known and can be correlated with an appropriate target analyte or target reaction product. In one aspect, all capture oligonucleotides in a particular binding domain have the same sequence and the capture oligonucleotides in one binding domain have a sequence different from capture oligonucleotides in other binding domains. In one aspect, multiple binding domains are arrayed in orderly rows and columns on a support surface and the precise location and sequence of each binding domain is recorded in a computer database. In one aspect, the array is arranged in a symmetrical grid pattern. In other aspects, the array is arranged another pattern, including, but not limited to, radially distributed lines, spiral lines, or ordered clusters. In another aspect, each binding domain is positioned on a surface of one or more microparticles or beads wherein the microparticles or beads are coded to allow for discrimination between different binding domains.

In one aspect, the support surface is a multi-well plate that includes one or more discrete addressable binding domains within each well that correspond to one or more capture oligonucleotides. In one aspect, the support surface includes at least one binding domain for detecting a wild type nucleotide sequence and separate binding domain for detecting a mutant nucleotide sequence. In one aspect, each well includes at least 2, 3, 4, 5, 6, 7, 8, 9, 10, 11, 12, 13, 14, 15, 16, 17, 18, 19, 20, 21, 22, 23, 24 or 25 binding domains. In one aspect, each well includes at least 7, 10, 16, or 25 binding domains.

In one aspect, the support surface is a multi-well plate that includes at least 24, 96, or 384 wells and each well includes array of up to 10 binding domains in which different capture oligonucleotides are immobilized in discrete binding domains. In a more particular aspect, the support surface is a 96 well plate in which each well includes an array having up to 10 binding domains. In one aspect, each well of a 96-well plate includes up to 10 binding domains, having up to 10 distinct capture oligonucleotides immobilized thereon. In one aspect, each well includes the same patterned array with the same capture oligonucleotides. In another aspect, different wells may include a different patterned array of capture oligonucleotides.

### E. Electrodes

In one aspect, a target analyte, including, for example, a polypeptide or nucleic acid sequence, is identified, detected or quantified using electrochemiluminescence. Multiplexed measurement of analytes using electrochemiluminescence is described in U.S. Pat. Nos. 7,842,246 and 6,977,722, the disclosures of which are incorporated herein by reference in their entireties.

In one aspect, the support surface includes one or more electrodes. In one aspect, the support surface includes one or more working electrodes and one or more counter electrodes. In one aspect, the support surface includes one or more binding domains formed on one or more electrodes for use in electrochemical or electrochemiluminescence assays.

In one aspect, the binding domains are formed by collecting beads coated with capture oligonucleotides onto the electrode surface. In one aspect, the beads are paramagnetic and the beads are collected on the electrode through the use of a magnetic field.

In one aspect, one or more capture oligonucleotides are covalently or non-covalently immobilized on one or more binding domains on one or more electrodes on the support surface. In one aspect, multiple distinct binding domains are present on one or more electrodes for multiplexed measurement of target analytes in a sample.

In one aspect, the electrodes are provided within an assay module that provides assay containers, assay flow cells, assay fluidics or other components useful for carrying out an assay. Examples of assay modules for carrying out electrochemiluminescence assays include, for example, multiarray case, assay plates case, cartridge case, and the like. In one aspect, the electrodes are provided within an assay module that provides assay containers, assay flow cells, assay fluidics or other components useful for carrying out an assay. Examples of assay modules for carrying out electrochemiluminescence assays can be found in U.S. Pat. Nos. 6,673,533, 7,842,246, 9,731,297, and 8,298834. In one aspect, the support surface is multi-well plate that includes at least one electrode. In one aspect, each well of a multi-well assay plate includes at least one electrode. In one aspect, at least one well of the multi-well assay plate includes a working electrode. In another aspect, at least one well of the multi-well assay plate includes a working electrode and a counter electrode. In another aspect, each well of the multi-well assay plate includes a working electrode and a counter electrode. In one aspect, the working electrode is adjacent, but not in electrical contact with the counter electrode.

In one aspect, the electrodes are constructed from a conductive material, including, for example, a metal such as gold, silver, platinum, nickel, steel, iridium, copper, aluminum, a conductive alloy, or combinations thereof. In another aspect, the electrodes include semiconducting materials such as silicon and germanium or semi-conducting films such as indium tin oxide (ITO) and antimony tin oxide (ATO). In another aspect, the electrodes include oxide coated metals, such as aluminum oxide coated aluminum. In one aspect, the electrode includes a carbon-based material. In one aspect the electrodes include mixtures of materials containing conducting composites, inks, pastes, polymer blends, and metal/non-metal composites, including for example, mixtures of conductive or semi-conductive materials with non-conductive materials. In one aspect, the electrodes include carbon-based materials such as carbon, glassy carbon, carbon black, graphitic carbon, carbon nanotubes, carbon fibrils, graphite, carbon fibers and mixtures thereof. In one aspect, the electrodes include conducting carbon-polymer composites, conducting polymers, or conducting particles dispersed in a matrix, for example, carbon inks, carbon pastes, or metal inks. In one aspect, the working electrode is made of a carbon-polymer composite that includes, for example, conducting carbon particles, such as carbon fibrils, carbon black, or graphitic carbon, dispersed in a matrix, for example, a polymer matrix such as ethylene vinyl acetate (EVA), polystyrene, polyethylene, polyvinyal acetate, polyvinyl chloride, polyvinyl alcohol, acrylonitrile butadiene styrene (ABS), or copolymers of one or more of these polymers.

In one aspect, the working electrode is made of a continuous conducting sheet or a film of one or more conducting materials, which may be extruded, pressed or molded. In another aspect, the working electrode is made of a conducting material deposited or patterned on a substrate, for example, by printing, painting, coating, spin-coating, evaporation, chemical vapor deposition, electrolytic deposition, electroless deposition, photolithography or other electronics microfabrication techniques. Inone aspect, the working electrode includes a conductive carbon ink printed on a polymeric support, for example, by ink-jet printing, laser printing, or screenprinting. Carbon inks are known and include materials produced by Acheson Colloids Co. (e.g., Acheson 440B, 423ss, PF407A, PF407C, PM-003A, 30D071, 435A, Electrodag 505SS, and Aquadag™), E. I. Du Pont de Nemours and Co. (e.g., Dupont 7105, 7101, 7102, 7103, 7144, 7082, 7861D, and CB050), Conductive Compounds Inc (e.g., C-100), and Ercon Inc. (e.g., G-451).

In one aspect, the working electrode is a continuous film. In another aspect, the working electrode includes one or more discrete regions or a pattern of discrete regions. Alternately, the working electrode may include a plurality of connected regions. One or more regions of exposed electrode surface on a working electrode can be defined by a patterned insulating layer covering the working electrode, for example, by screen printing a patterned dielectric ink layer over a working electrode, or by adhering a die-cut insulating film. The exposed regions may define the array elements of arrays of reagents printed on the working electrode and may take on array shapes and patterns as described above. In one aspect, the insulating layer defines a series of circular regions (or “spots”) of exposed working electrode surface.

A counter electrode may have one or more of the properties described above generally for working electrodes. In one aspect, the working and counter electrodes are constructed from the same material. In another aspect, the working and counter electrodes are not constructed from the same material, for example, the working electrode may be a carbon electrode and the counter electrode may be a metal electrode.

In one aspect, one or more capture oligonucleotides are immobilized on one or more electrodes by passive adsorption. In another aspect, one or more capture oligonucleotides are covalently immobilized on the electrodes. In one aspect the electrodes are derivatized or modified, for example, to immobilize reagents such as capture oligonucleotides on the surface of the electrodes. In one aspect, the electrode is modified by chemical or mechanical treatment to improve the immobilization of reagents, for example, to introduce functional groups for immobilization of reagents or to enhance its adsorptive properties. Examples of functional groups that can be introduced include, but are not limited, to carboxylic acid (COOH), hydroxy (OH), amino (NH₂), activated carboxyls (e.g., N-hydroxy succinimide (NHS)-esters), polyethylene glycols), thiols, alkyl ((CH₂)ₙ) groups, or combinations thereof). In one aspect, one or more reagents, for example, capture oligonucleotides, are immobilize by either covalent or non-covalent means to a carbon-containing electrode, for example, carbon black, fibrils, or carbon dispersed in another material. It has been found that capture molecules having thiol groups can bind covalently to carbon-containing electrodes, for example to screen-printed carbon ink electrodes, without having to first deposit an additional thiol-reactive layer such as a protein layer or a chemical cross-linking layer. In one aspect, methods are provided for direct attachment of capture molecules having thiol groups, such as thiol-modified oligonucleotides, to electrodes which provide simple, robust, efficient and reproducible processes for forming capture surfaces and arrays on electrodes. In one aspect, one or more capture oligonucleotides having thiol groups are directly immobilized on carbon-containing electrodes, such as screen-printed carbon ink electrodes, through reaction of the thiols with the electrode, without first adding a thiol-reactive layer to the electrode.

In one aspect the electrode is treated with a plasma, for example, a low temperature plasma, such as a glow-discharge plasma, to alter the physical properties, chemical composition, or surface-chemical properties of the electrode, for example, to aid in the immobilization of reagents such as a capture oligonucleotide, or to reduce contaminants, improve adhesion to other materials, alter the wettability of the surface, facilitate deposition of materials, create patterns, or improve uniformity. Examples of useful plasmas include oxygen, nitrogen, argon, ammonia, hydrogen, fluorocarbons, water and combinations thereof. In one aspect, oxygen plasma is used to treat an electrode with carbon particles in a carbon-polymer composite material. In another aspect, oxygen is used to introduce carboxylic acids or other oxidized carbon functionality into carbon or organic materials (for example, activated esters or acyl chlorides) to facilitate coupling of reagents. In another aspect, ammonia-containing plasmas may be used to introduce amino groups for use in coupling assay reagents. In one aspect, the electrode is not pretreated to aid in the immobilization of one or more capture oligonucleotides.

In one aspect, the support surface includes an assay module such as a multi-well plate having one or more working or counter electrodes in each well. In one aspect, the multi-well plate includes a plurality of working or counter electrodes in each well. In one aspect, the working or counter electrodes of the multi-well plate include carbon, for example, screen-printed layers of carbon inks. In one aspect, one or more capture oligonucleotides are immobilized on the screen-printed carbon ink through a thiol moiety on the capture oligonucleotide. In one aspect, the working electrode is used to induce an electrochemiluminescent signal from a label that is attached to a reaction product. In one aspect, the electrochemiluminescent signal is emitted from ruthenium-tris-bipyridine in the presence of a co-reactant such as a tertiary alkyl amine, for example, tripropyl amine or butyldiethanolamine.

In one aspect, the electrode contains binding domains as described above that are defined by dielectric ink (i.e., electrically insulating ink). The electrode is a working electrode with a dielectric printed over it in a pattern that defines the binding domains described above. In one aspect, the binding domains are roughly circular areas of exposed working electrode (or “spots”). The electrodes are in 96-well plates formed by adhering an injection molded 96-well plate top to a mylar sheet that defines the bottom of the wells. The top surface of the mylar sheet has screen printed carbon ink electrodes printed on it such that each well includes a carbon ink working electrode roughly in the center of the well and two carbon ink counter electrodes roughly towards two edges of the well. The electrodes printed on the bottom of the mylar sheet, connected through conductive through-holes to the top of the sheet, provide contacts for applying electrical voltage to the working and counter electrodes.

### F. Methods of Immobilizing a Capture Molecule

In one aspect, a method of immobilizing one or more capture molecules on a support surface is provided. In one aspect, one or more capture molecules include one or more single stranded capture oligonucleotide molecules as described herein.

In one aspect, the method includes immobilizing one or more capture molecules on a support surface that includes a carbon-based support surface. In one aspect, the method includes immobilizing one or more capture molecules on a support surface that includes one or more electrodes. In one aspect, the method includes immobilizing one or more capture molecules on a support surface that includes one or more carbon-based electrodes.

In one aspect, the support surface is a multi-well plate that includes one or more electrodes. In one aspect, the support surface is a multi-well plate that includes one or more electrodes in each well. In one aspect, one or more capture molecules are immobilized on a support surface in an array.

In one aspect, the method includes spotting or printing two or more capture oligonucleotides in an array on a first electrode in a first well of the multi-well plate and subsequently printing one or more capture oligonucleotides in an array on an electrode in one or more additional wells of the multi-well plate. In one aspect, at least some of the printed arrays in each well are the same. In another aspect, at least some of the printed arrays in each well are different.

In one aspect, one or more capture molecules are spotted or printed at one or more known locations within the array, referred to as binding domains. In one aspect, one or more capture oligonucleotides are immobilized in discrete, non-overlapping, addressable binding domains and the sequence of the capture oligonucleotide in each binding domain is known and can be correlated with a target analyte. In one aspect, all capture oligonucleotides in a particular binding domain have the same sequence and the capture oligonucleotides in one binding domain have a sequence different from capture oligonucleotides in other binding domains.

In one aspect, one or more capture molecules are spotted or printed onto discrete binding domains on the support surface. In one aspect, an array of capture oligonucleotides is spotted or printed onto discrete binding domains on a support surface. In one aspect, the capture molecules are spotted or printed by contact printing, including, for example, contact pin printing or microstamping, or by non-contact printing, including, for example, photolithography, laser writing, electrospray deposition, and inkjet printing. In general, spotting or printing methods include applying one or more liquid droplets that include one or more capture molecules onto discrete binding domains on the support surface and allowing the liquid droplets to dry. In one aspect, the liquid droplets are allowed to spread to cover an area the support surface. In one aspect, the support surface includes one or more regions of higher wettability and one or more regions of lower wettability, wherein the regions of higher wettability define binding domains or array elements. Wettability refers to the interaction between a liquid and a solid surface, more particularly, to the phenomenon in which an aqueous solution does not spread onto a solid surface, but instead contracts to form droplets. In one aspect, the solid support has surface properties to encourage droplet formation when small volumes of an aqueous solution are dispensed onto one or more discrete binding domains. Solutions of capture molecules printed on the higher wettability regions spread to the boundaries with the lower wettability regions providing precise control over the shape and position of binding domains. In one aspect, the binding domains are regions of exposed electrode surface on a working electrode, and a patterned insulating layer on the working electrode (for example a screen-printed dielectric ink over a screen-printed carbon ink electrode) defines the lower wettability boundaries of the exposed electrode regions.

Methods for immobilizing oligonucleotides to a support surface are known (see, for example, Balasheb Nimse et al. (2014) Immobilization Techniques for Microarray: Challenges and Applications. Sensors. 14(2): 22208-22229) and are generally based on one or more of the following mechanisms: (1) physical adsorption, for example, via charge-charge or hydrophobic interactions (2) covalent immobilization, for example, via chemical bonding; and (3) non-covalent protein-ligand interactions such as streptavidin-biotin immobilization. In one aspect, one or more oligonucleotides are immobilized to a functionalized support surface. In one aspect, one or more oligonucleotides are immobilized to a support surface that has not been modified to include one or more functional groups. In one aspect, one or more oligonucleotides are immobilized by physical absorption to a support surface that includes one or more of the following moieties: amine, nitrocellulose, poly(l-lysine), PAAH, and diazonium. In one aspect, one or more oligonucleotides are immobilized to a support surface by covalent interactions, for example through a thiol (—SH), amine (—NH₂), or hydrazide group. In one aspect, the support surface includes or is modified to include a reactive functionality, including, for example, carboxyl (—COOH), aldehyde (—CHO), epoxy (—CHCH₂O), isothiocyanate (—N═C═S), maleimide (—HC₂(CO)₂NH), or mercaptosilane (—Si—R—SH). In one aspect, the oligonucleotide includes or is modified to include a reactive functionality, including, for example, a thiol, amine or hydrazide group. In one aspect, one or more oligonucleotides are immobilized to a support surface through a nucleophilic or electrophilic functionality present on the support surface.

In one aspect, one or more capture molecules include a thiol group. In one aspect, one or more capture molecules are immobilized on the support surface through a thiol group present on the capture molecule. In one aspect, the method includes spotting or printing one or more capture molecules that include a thiol group onto a carbon-based support surface and incubating the printed support surface to immobilize one or more capture molecules on the support surface through the thiol group. In one aspect, one or more capture molecules are covalently attached to the support surface through the thiol group.

In one aspect, one or more capture molecules are immobilized onto a support surface by printing liquid droplets (e.g., 50 nL) that contain the capture molecules onto the support surface, allowing the liquid droplets to spread, allowing the liquid droplets to dry, and incubating the dried droplets for an amount of time sufficient to immobilize the capture molecules to the support surface (e.g., overnight). In one aspect, one or more capture molecules that include a thiol group are immobilized onto a carbon-based support surface by printing liquid droplets that contain the capture molecules onto the support surface, allowing the liquid droplets to spread, allowing the liquid droplets to dry and incubating the dried droplets for an amount of time sufficient to immobilize the capture molecules to the support surface through the thiol groups. In one aspect, the liquid droplets are printed in an array. In one aspect, the liquid droplets are printed in one or more binding domains. In one aspect, the carbon-based support surface includes one or more carbon-based electrodes. In one aspect, one or more capture molecules are covalently attached to the carbon-based electrodes through a thiol group. In one aspect, a patterned insulating layer is included on the carbon-based support surface to delimit the spread of liquid droplets printed on the support surface.

In one aspect, the carbon-based support surface is pretreated, for example, to introduce one or more functional groups on the support surface, for example, to increase reactivity between the thiol group on the capture molecule and the support surface. In one aspect, the carbon-based support surface is pretreated with a protein such as bovine serum albumin (BSA). In another aspect, the carbon-based support surface is not pretreated to introduce any functional groups on the support surface before immobilizing one or more capture oligonucleotides to the support surface through the thiol group. In one aspect, the support surface is not modified with a protein to increase reactivity of the thiol group on the capture molecule and the support surface.

In one aspect, the support surface is washed with a wash (or blocking) solution after one or more capture oligonucleotides are spotted or printed on to the surface to remove free capture oligonucleotide (i.e., capture oligonucleotides that are not immobilized to the support surface) (also referred to herein as a “blocking” step; see, e.g., Example 3). In one aspect, the support surface is washed with a wash solution after printing and drying. In one aspect, the support surface is washed before it is packaged in a desiccated package. In another aspect, the support surface is washed after it is packaged in a desiccated package.

In one aspect, the washing or blocking step comprises adding the wash or blocking solution to the surface (e.g., 50 uL of solution per well for a 96-well assay plate) and incubating for 30 to 60 minutes. The incubation temperature may be any convenient temperature, e.g., room temperature or 37° C. The incubation may take place while shaking the surface. The wash or blocking step may comprise removing the wash or blocking solution and rinsing the surface with a buffer such as PBS.

In one aspect, the wash solution includes a thiol-containing compound. During the wash step, excess thiol-containing capture molecules from one binding domain on a carbon-based electrode can transfer to another binding domain and become permanently affixed. This transfer of capture molecules, and the resulting cross-contamination of binding domains, can be reduced by including a thiol-containing compound in the wash solution. While not wishing to be bound by theory, it is believed that the thiol-containing compound in the wash solution competes with the free (unbound) capture oligonucleotide and prevents cross-contamination of binding domains from the binding of excess capture oligonucleotide that is removed from a different binding domain. In one aspect, the wash solution includes a water-soluble thiol-containing compound. In one aspect, the wash solution includes a water-soluble thiol-containing compound having a molecular weight of less than about 200 g/mol, about 175 g/mol, about 150 g/mol or about 125 g/mol. In one aspect, the water-soluble thiol-containing compound includes a zwitterion.

In one aspect, the wash solution includes a water-soluble thiol selected from cysteine (e.g., L-cysteine), cysteamine, dithiothreitol, 3-mercaptoproprionoate, and 3-mercapto-1-propanesulfonic acid. In one aspect, the water-soluble thiol containing compound includes cysteine.

In one aspect, the wash solution includes a pH buffering component. In one aspect, the pH buffering component includes Tris. In one aspect, the wash solution includes a surfactant. In one aspect, the surfactant includes Triton X-100. In one aspect, the wash solution includes a metal chelating agent.

In one aspect, the wash solution includes between about 5 mM and about 750 mM, between about 10 mM and about 500 mM, about 25 mM and about 75 mM, or about 50 mM of the thiol-containing compound. In one aspect, the wash solution includes between about 5 mM and about 750 mM, between about 10 mM and about 500 mM, about 25 mM and about 75 mM, or about 50 mM cysteine. In one aspect, the wash solution includes between about 10 mM and about 30 mM, or about 15 mM and about 25 mM, or about 20 mM of a buffer such as Tris. In one aspect, the wash includes between about 0.05% and about 0.5%, or between about 0.05% and 0.2%, or about 0.1% of a surfactant such as Triton X-100. In one aspect, the wash solution has a pH between about 7 and about 9, about 7.5 and about 8.5, or about 8.0.

In one aspect, the wash or blocking solution includes one or more of the following reagents: (i) known polymers useful for reducing background signals in hybridization assays, including, but not limited to, PS20, polyvinyl alcohol (PVA), polyvinylpyrrolidone (~ 1,000 kD or ~ 360 kD), Ficoll, and polyethylene glycol (~ 3 kD and ~ 10 kD), (ii) nucleic acids or other polyanions including, but not limited to, salmon sperm DNA, herring DNA, calf thymus DNA, sheared PolyA, yeast tRNA; and heparin, (iii) monomeric and polymeric protein blocking agents including, but not limited to, BSA and poly-BSA, (iv) surfactants, including, but not limited to, sodium dodecyl sulfate (SDS), 3-[(3-cholamidopropyl)dimethylammonio]-1-propanesulfonate (CHAPS), triton-100, and tween-20, and (v) hydrogen bond destabilizers, including, but not limited to, formamide and propylene glycol.

In one aspect, the method includes a step of immobilizing one or more capture oligonucleotides on a support surface and then washing excess non-immobilized capture oligonucleotide off the support surface with a wash solution. In one aspect, washing includes washing the immobilized capture oligonucleotides under stringent wash conditions. In one aspect, the stringent wash conditions include a temperature of between about 27° C. and about 47° C., a formamide concentration between about 21% and about 41%, a salt concentration between about 300 mM and about 500 mM and a pH between about 7.5 and about 8.5. In one aspect, the high stringency conditions include a temperature of about 37° C., a formamide concentration of about 31%, a salt concentration of about 400 mM and a pH of 8.0. In one aspect, the immobilized oligonucleotides are exposed to high stringency conditions for at least 5, 10, 30 or 60 minutes. In another aspect, the high stringency condition includes a low salt condition, for example, a buffer with a salt concentration of less than about 40 mM, 20 mM, 15 mM, or 10 mM. In one aspect, the high stringency conditions include a low salt condition such as 0.1X PBS at 37° C.

In one aspect, one or more capture oligonucleotides are immobilized on the support surface in an array. In one aspect, one or more capture oligonucleotides are immobilized on the support surface in one or more binding domains. In one aspect, the capture oligonucleotides printed on one binding domain of the array have a different sequence than capture oligonucleotides printed on other binding domains in the array.

While not wishing to be bound by theory, it is believed that the wash solution brings loosely bound capture oligonucleotides into solution, from which they can potentially be re-deposited to the surface either via SH-covalent binding or other mechanisms. If a capture oligonucleotide is re-deposited on a binding domain with capture oligonucleotides having a different nucleotide sequence, it is considered a contaminating capture molecule. The presence of contaminating capture molecules can interfere with the assay results. In one aspect, the binding domains of an array prepared by the methods described herein include less than about 0.1%, 0.09%, 0.08%, 0.07%, 0.06%, 0.05%, 0.04%, 0.03%, 0.02%, or 0.01% contaminating capture molecules.

In one aspect, cross-reactivity between the binding partners (i.e., oligonucleotide tags) of a set of capture molecules is less than about 0.1%, 0.09%, 0.08%, 0.07%, 0.06%, 0.05%, 0.04%, 0.03%, 0.02%, or 0.01%. In one aspect, assay specificity (including cross-reactivity from either binding of non-complementary sequences or from capture oligonucleotide cross-contamination) is determined. In one aspect, specificity is determined by adding one or more samples containing one or more labeled QC probes to one or more replicate plates under conditions in which the QC probes hybridize to their corresponding complementary capture molecules immobilized on the plate surface. The plates are then washed to remove excess QC probe and the presence of bound QC probe is detected, either by detection of a primary label or by the addition of a secondary binding partner. Cross-reactivity can be calculated for each array, for example, for each well in a multi-well plate, as the signal detected from the binding of a probe to a spot with a non-specific capture nucleotide as a percentage of the signal from the binding of the probe to the spot with its corresponding complementary capture nucleotide. In one aspect, the calculation includes a correction for non-specific background signal detected in the absence of any QC probe.

In one aspect, assay specificity is determined using a set of quality control (QC) oligonucleotide probes. In one aspect, the QC probes include nucleotide sequences complementary to at least 20, 21, 22, 23, 24, 25, 26, 27, 28, 29, 30, 31, 32, 33, 34, 35 or 36 consecutive nucleic acids of a corresponding capture molecule in a set of non-cross-reactive capture molecules immobilized on a surface. In one aspect, the set includes QC probes having nucleotide sequences complementary to at least 20, 21, 22, 23, 24, 25, 26, 27, 28, 29, 30, 31, 32, 33, 34, 35 or 36 consecutive nucleic acids of a corresponding capture molecule in a set of non-cross-reactive capture molecules with a sequence shown in any of SEQ ID NOs: 1-774. In one aspect, the set includes QC probes having nucleotide sequences complementary to at least 20, 21, 22, 23, 24, 25, 26, 27, 28, 29, 30, 31, 32, 33, 34, 35 or 36 consecutive nucleic acids of a corresponding capture molecule in a set of non-cross-reactive capture molecules shown in SEQ ID NOs: 1-64. In one aspect, the set includes QC probes having nucleotide sequences complementary to at least 20, 21, 22, 23, 24, 25, 26, 27, 28, 29, 30, 31, 32, 33, 34, 35 or 36 consecutive nucleic acids of a corresponding capture molecule in a set of non-cross-reactive capture molecules shown in SEQ ID NOs: 1-10.

In one aspect, the QC probes include a label. In one aspect, the label is attached directly to the QC probe. In another aspect, the label is attached to the QC probe through a linker. In one aspect, the label is a compound that is a member of a binding pair, in which a first member of the binding pair (which can be referred to as a “primary binding reagent”) is attached to a substrate, for example, an oligonucleotide, and the other member of the binding pair (which can be referred to as a “secondary binding reagent”) has a detectable physical property. Examples or primary labels include, but are not limited to, an electrochemiluminescence label, an organometallic complex that includes a transition metal, for example, ruthenium. In one aspect, the primary label includes streptavidin. In one aspect, the primary label includes MSD SULFO-TAG labeled streptavidin.

In one aspect, the label includes a secondary binding reagent that binds to the primary binding reagent. In one aspect, the primary binding reagent includes biotin, a hapten, streptavidin, avidin or antibody or antigen. In one aspect, the secondary binding reagent includes biotin, a hapten, streptavidin, avidin or antibody or antigen. In one aspect, the secondary binding reagent includes an electrochemiluminescence label. In one aspect, the secondary binding reagent includes an organometallic complex that includes a transition metal, for example, ruthenium. In one aspect, QC probes include biotin and the secondary binding reagent includes MSD SULFO-TAG labeled streptavidin. In one aspect, the QC probes are modified at the 3′ end with biotin as shown in the structure below:

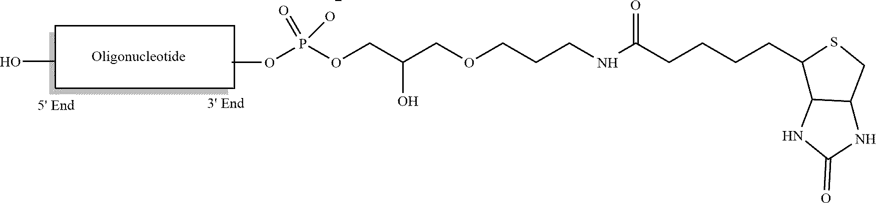

In one aspect, the percent of contaminating capture molecules is measured by the method of Example 4.

In one aspect, the uniformity of one or more binding domains on a plate (intraplate) or across two or more plates (interpolate) can be determined using known methods for determining the coefficient of variation (CV). In one aspect, the intraplate or interplate binding domains have a CV of less than about 10%, 9.5%, 9%, 8.5%, 8%, 7.5%, 7%, 6.5%, 6%, 5.5%, 5%, 4.5%, 4%, 3.5%, 3%, 2.5%, 2%, 1.5%, or 1%. In one aspect, the average intraplate or interplate CV is between about 3% and about 6%, or less than about 5%. In one aspect, binding domain uniformity is measured by the method of Example 5.

### G. Aptamer Immobilization

In one aspect, a method of immobilizing one or more aptamers on a support surface is provided. In one aspect, one or more aptamers are immobilized onto a support surface by binding to one or more single stranded capture molecules that are immobilized to the support surface as described herein. In one aspect, the aptamer is an oligonucleotide that is capable of specifically binding to a target molecule and can include, for example, DNA, RNA or XNA aptamers which bind to molecular targets, including, for example, small molecules, proteins, nucleic acids, cells, tissues and organisms non-covalent interactions, such as electrostatic and hydrophobic interactions. In another aspect, the aptamer is a peptide that is capable of specifically binding to a target molecule that includes at least one or more variable peptide domains displayed by a protein scaffold. In one aspect, the immobilized aptamers are used as probes for one or more target analytes. In one aspect, the immobilized aptamers are used in a microarray.

### H. Oligonucleotide Probe

In one aspect, the method or kit includes one or more probe reagents that are capable of specifically binding to a target analyte in a sample. In one aspect, the oligonucleotide probe includes a binding partner that is capable of specifically binding to a target analyte in a sample.

As used herein, the term “binding partner” refers to a member of a pair of moieties that specifically bind to each other under a particular set of conditions, that is the binding pair bind to each other to the substantial exclusion of other moieties present in the environment. A binding partner can be any molecule, such as a polypeptide, lipid, glycolipid, nucleic acid molecule, carbohydrate or other molecule, with which another molecule specifically interacts, for example, through covalent or noncovalent interactions, including, for example, the interaction of an antibody with its cognate antigen, the interaction between two complementary nucleotide sequences, or the interaction between biotin and streptavidin or avidin. The term “corresponding” refers to the relationship between two specific binding partners, such that one member of a binding partner pair “corresponds” to the other member of the pair.

In one aspect, the binding partner includes an antibody that specifically binds to the target analyte. In another aspect, the binding partner includes an oligonucleotide sequence that is complementary to an oligonucleotide sequence of the target analyte such that the oligonucleotide probe is capable of hybridizing to the target nucleotide sequence.

In one aspect, the oligonucleotide probe includes an oligonucleotide tag and a binding partner. In one aspect, the binding partner includes a single stranded sequence that is complementary or substantially complementary to a portion of a target nucleotide sequence. In one aspect, the probe includes an oligonucleotide tag having a sequence that is complementary to a sequence of a capture oligonucleotide. In one aspect, the oligonucleotide tag and the binding partner are different regions of a single oligonucleotide strand.

In one aspect, the probe is a single stranded nucleic acid sequence, including, for example, nucleic acid sequences including deoxyribonucleic acids (DNA) or ribonucleic acids (RNA), peptide nucleic acids (PNA) or locked nucleic acids (LNA). In one aspect, the probe includes one or more modified nitrogenous bases analogs or bases that have been modified to include a label or a reactive functional group or linker suitable for attaching a label.

In one aspect, the probe is between about 5 and about 100, about 10 and about 50, about 20 and about 30, or at least about 5, 6, 7, 8, 9, 10, 15, 20 or 25 and up to about 30, 35, 40, 45, 50, 75 or 100 nucleotides in length. Probes can be prepared by any suitable method known in the art, including chemical or enzymatic synthesis or by cleavage of larger nucleic acids using non-specific nucleic acid-cleaving chemicals or enzymes, or with site-specific restriction endonucleases. In some applications, a probe that is hybridized to a complementary region in a target sequence can prime extension of the probe by a polymerase, acting as a starting point for replication of adjacent single stranded regions on the target sequence.

In one aspect, the probe includes a label. In one aspect, the label is attached directly to the probe. In another aspect, the label is attached to the probe through a linker. In one aspect, the label is a compound that is a member of a binding pair, in which a first member of the binding pair (which can be referred to as a “primary binding reagent”) is attached to a substrate, for example, an oligonucleotide, and the other member of the binding pair (which can be referred to as a “secondary binding reagent”) has a detectable physical property. Examples or primary labels include, but are not limited to, an electrochemiluminescence label, an organometallic complex that includes a transition metal, for example, ruthenium. In one aspect, the primary label is the MSD SULFO-TAG label.

In one aspect, a secondary binding reagent binds to the primary binding reagent. In one aspect, the primary binding reagent includes biotin, a hapten, streptavidin, avidin or antibody or antigen. In one aspect, the secondary binding reagent includes biotin, a hapten, streptavidin, avidin or antibody or antigen. In one aspect, the secondary binding reagent includes an electrochemiluminescence label. In one aspect, the secondary binding reagent includes an organometallic complex that includes a transition metal, for example, ruthenium. In one aspect, the secondary binding reagent includes the MSD SULFO-TAG label.

In one aspect, the kit includes one or more probe reagents. In one aspect, the end user prepares one or more probe reagents.

### I. Oligonucleotide Tags

In one aspect, the probe includes an oligonucleotide tag having a sequence that specifically binds to an oligonucleotide sequence of a capture molecule. In one aspect, the tag includes a single stranded oligonucleotide that is complementary to at least a portion of the nucleotide sequence of a single stranded capture oligonucleotide. In one aspect, the oligonucleotide tag is recombinantly produced. In one aspect, the oligonucleotide tags are not naturally occurring sequences. In one aspect, one or more capture oligonucleotides include single stranded nucleic acid sequences, including for example, nucleic acid sequences including deoxyribonucleic acids (DNA), ribonucleic acids (RNA), or structural analogs that include non-naturally occurring chemical structures that can also participate in hybridization reactions.

In one aspect, the tag is attached to the 5′-end of the probe. In another aspect, the tag is attached to the 3′-end of the probe. In one aspect, the tag is not complementary to and does not hybridize with the target nucleotide sequence.

In one aspect, the sequence that is complementary to the target nucleotide sequence and the oligonucleotide tag sequence are present on one nucleic acid strand within a probe. In another aspect, the sequence that is complementary to the target nucleotide sequence and the oligonucleotide tag sequence are present on different nucleic acid strands. In one aspect, the probe includes a first strand having a sequence complementary to the target sequence and a first bridging sequence and a second strand having an oligonucleotide tag sequence and a second bridging sequence complementary to the first bridging sequence, wherein the first and second strands are hybridized or can hybridize through the first and second bridging sequences.

In one aspect, the oligonucleotide tag includes a label. In one aspect, the label is attached directly to the oligonucleotide tag. In another aspect, the label is attached to the oligonucleotide tag through a linker. In one aspect, the label is attached to the 5′ terminal nucleotide of the oligonucleotide tag. In another aspect, the label is attached to the 3′ terminal nucleotide of the oligonucleotide tag. In one aspect, the label is attached along the length of the oligonucleotide tag.

In one aspect, the label includes a radioactive, fluorescent, chemiluminescent, electrochemiluminescent, light absorbing, light scattering, electrochemical, magnetic or enzymatic label. In one aspect, the label includes an electrochemiluminescent label. In one aspect, the label includes a hapten. In one aspect, label is biotin, fluorescein or digoxigenin. In one aspect, the label includes an organometallic complex that includes a transition metal. In one aspect, the transition metal includes ruthenium. In one aspect, the label is a MSD SULFO-TAG™ label.

In one aspect, the oligonucleotide tag includes a primary binding reagent as a label, wherein the primary binding reagent is a binding partner of a secondary binding reagent. In one aspect, the primary binding reagent includes biotin, streptavidin, avidin, or an antigen. In one aspect, the secondary binding reagent includes biotin, streptavidin, avidin, or an antibody. In one aspect, the primary binding reagent includes an oligonucleotide and the secondary binding reagent is an oligonucleotide having a sequence that is complementary to the sequence of the primary binding reagent.

In one aspect, the tag has a nucleotide sequence that is at least about 15, 16, 17, 18, 19, 20, 21, 22, 23, 24 or 25 and up to about 25, 26, 27, 28, 29, 30, 31, 32, 33, 34, 35, 36, 37, 38, 39 or 40, or between about 15 and about 40, or about 20 and about 30 nucleotides in length. In one aspect, the tag includes a nucleotide sequence that is at least about 1, 2, 3, 4, 5, 6, 7, 8, 9, or 10 and up to about 11, 12, 13, 14, 15, 16, 17, 18, 19, or 20, or between about 1 and about 20 or between about 10 and about 15 or between about 12 and about 13 nucleotides shorter than the complementary capture oligonucleotide sequence. In one aspect, the tag has a nucleotide sequence that is at least about 24, 30 or 36 nucleotides in length.

In one aspect, the oligonucleotide tag has a sequence that hybridizes to a capture molecule having a sequence shown in any of SEQ ID NOs: 1-774 (Tables 1-12). In one aspect, the oligonucleotide tag has a sequence that hybridizes to a complementary capture molecule having a sequence shown in any of SEQ ID NOs: 1-744 (Tables 1-12). In one aspect, the tag has a nucleotide sequence is complementary to a sequence that is at least about 20, 21, 22, 23, 24, 25, 26, 27, 28, 29, 30, 31, 32, 33, 34, 35 or 36 consecutive nucleotides of a sequence shown in any of SEQ ID NOs: 1-744. In one aspect, the tag has a nucleotide sequence that is complementary to a sequence that is at least about 24, 30 or 36 consecutive nucleotides of a sequence shown in any of SEQ ID NOs: 1-744. In one aspect, the oligonucleotide tag has a nucleic acid sequence shown in any of SEQ ID NOs: 745-1488 (Tables 13-24).

In one aspect, the oligonucleotide tag has a nucleotide sequence that is at least 95%, 96%, 97%, 98%, 99% or 100% identical to a sequence shown in any of SEQ ID NOs: 745-1488 (Tables 13-24). In another aspect, the oligonucleotide tag has a nucleotide sequence that includes at least 20, 21, 22, 23, 24, 25, 26, 27, 28, 29, 30, 31, 32, 33, 34, 35 or 36 consecutive nucleotides of a sequence shown in any of SEQ ID NOs: 745-1488 (Tables 13-24). In another aspect, the oligonucleotide tag has a nucleotide sequence that includes at least 20 consecutive nucleotides of a sequence shown in any of SEQ ID NOs: 745-1488 (Tables 13-24). In another aspect, the oligonucleotide tag has a nucleotide sequence that includes at least 20, 21, 22, 23, 24, 25, 26, 27, 28, 29, 30, 31, 32, 33, 34, 35 or 36 consecutive nucleotides of a sequence that is at least 95%, 96%, 97%, 98%, 99% or 100% identical to a nucleotide sequence in any of SEQ ID NOs: 745-1488 (Tables 13-24). In another aspect, the oligonucleotide tag has a nucleotide sequence that includes at least 20 consecutive nucleotides of a sequence that is at least 95%, 96%, 97%, 98%, 99% or 100% identical to a nucleotide sequence in any of SEQ ID NOs: 745-1488 (Tables 13-24).

In one aspect, the oligonucleotide tag has a nucleotide sequence that is at least 95%, 96%, 97%, 98%, 99% or 100% identical to a sequence shown in any of SEQ ID NOs: 745-754, 755-757, 769-770, 777-781, 786, 788-790, 798 and 803-806. In another aspect, the oligonucleotide tag has a nucleotide sequence that includes at least 20, 21, 22, 23, 24, 25, 26, 27, 28, 29, 30, 31, 32, 33, 34, 35 or 36 consecutive nucleotides of a sequence shown in any of SEQ ID NOs: 745-754, 755-757, 769-770, 777-781, 786, 788-790, 798 and 803-806. In another aspect, the oligonucleotide tag has a nucleotide sequence that includes at least 20 consecutive nucleotides of a sequence shown in any of SEQ ID NOs: 745-754, 755-757, 769-770, 777-781, 786, 788-790, 798 and 803-806. In another aspect, the oligonucleotide tag has a nucleotide sequence that includes at least 20, 21, 22, 23, 24, 25, 26, 27, 28, 29, 30, 31, 32, 33, 34, 35 or 36 consecutive nucleotides of a sequence that is at least 95%, 96%, 97%, 98%, 99% or 100% identical to a nucleotide sequence in any of SEQ ID NOs: 745-754, 755-757, 769-770, 777-781, 786, 788-790, 798 and 803-806. In one aspect, the oligonucleotide tag has a nucleotide sequence shown in any of SEQ ID NOs: 745-754, 755-757, 769-770, 777-781, 786, 788-790, 798 and 803-806.

In one aspect, the oligonucleotide tag has a nucleotide sequence that is at least 95%, 96%, 97%, 98%, 99% or 100% identical to a sequence shown in any of SEQ ID NOs: 745-754. In another aspect, the oligonucleotide tag has a nucleotide sequence that includes at least 20, 21, 22, 23, 24, 25, 26, 27, 28, 29, 30, 31, 32, 33, 34, 35 or 36 consecutive nucleotides of a sequence shown in any of SEQ ID NOs: 745-754. In another aspect, the oligonucleotide tag has a nucleotide sequence that includes at least 20, 21, 22, 23, 24, 25, 26, 27, 28, 29, 30, 31, 32, 33, 34, 35 or 36 consecutive nucleotides of a sequence that is at least 95%, 96%, 97%, 98%, 99% or 100% identical to a nucleotide sequence in any of SEQ ID NOs: 745-754. In one aspect, the oligonucleotide tag has a nucleotide sequence shown in any of SEQ ID NOs: 745-754.

In one aspect, the method or kit includes a set of non-cross-reactive oligonucleotide tags selected from a “parent set” of non-cross-reactive oligonucleotide tags. In one aspect, the set of non-cross-reactive oligonucleotide tags are complementary to a set of non-cross-reactive capture oligonucleotides. In one aspect, the non-cross-reactive oligonucleotide tags in a set are configured to hybridize to their corresponding complementary sequences in a corresponding set of capture oligonucleotides. In one aspect, the oligonucleotide tags in a set hybridize to the non-complementary sequences in a corresponding set of capture oligonucleotides less than 0.05% relative to the complementary sequences.

Two or more oligonucleotides from a parent set can be selected to form a “subset” of non-cross-reactive oligonucleotide tags, wherein each oligonucleotide in the subset is a member of the original parent set. A subset cannot include oligonucleotide tags from more than one parent set. In one aspect, the set or subset of non-cross-reactive oligonucleotide tags includes at least 2, 3, 4, 5, 6, 7, 8, 9, 10, 11, 12, 13, 14, 15, 16, 17, 18, 19, 20, 21, 22, 23, 24 or 25 and up to 64 non-cross-reactive sequences selected from a parent set of non-cross-reactive sequences.

In one aspect, a first set of non-cross-reactive oligonucleotide tags is generated that is complementary to one or more capture sequences shown in Table 1 (SEQ ID NOs: 1-64). In one aspect, the first set of non-cross-reactive oligonucleotide tags includes two or more oligonucleotide tags from a parent set shown in Table 13 (SEQ ID NOs: 745-808).

In one aspect, a second set of non-cross-reactive oligonucleotide tags is generated that is complementary to one or more capture sequences shown in Table 2 (SEQ ID NOs: 65-122). In one aspect, the second set of non-cross-reactive oligonucleotide tags includes two or more oligonucleotide tags from a parent set shown in Table 14 (SEQ ID NOs: 809-866).

In one aspect, a third set of non-cross-reactive oligonucleotide tags is generated that is complementary to one or more capture sequences shown in Table 3 (SEQ ID NOs: 123-186). In one aspect, the third set of non-cross-reactive oligonucleotide tags includes two or more oligonucleotide tags from a parent set shown in Table 15 (SEQ ID NOs: 867-930).

In one aspect, a fourth set of non-cross-reactive oligonucleotide tags is generated that is complementary to one or more capture sequences shown in Table 4 (SEQ ID NOs: 187-250). In one aspect, the fourth set of non-cross-reactive oligonucleotide tags includes two or more oligonucleotide tags from a parent set shown in Table 16 (SEQ ID NOs: 931-994).

In one aspect, a fifth set of non-cross-reactive oligonucleotide tags is generated that is complementary to one or more capture sequences shown in Table 5 (SEQ ID NOs: 251-308). In one aspect, the second set of non-cross-reactive oligonucleotide tags includes two or more oligonucleotide tags from a parent set shown in 17 (SEQ ID NOs: 995-1052).

In one aspect, a sixth set of non-cross-reactive oligonucleotide tags is generated that is complementary to one or more capture sequences shown in Table 6 (SEQ ID NOs: 309-372). In one aspect, the second set of non-cross-reactive oligonucleotide tags includes two or more oligonucleotide tags from a parent set shown in Table 18 (SEQ ID NOs: 1053-1116).

In one aspect, a seventh set of non-cross-reactive oligonucleotide tags is generated that is complementary to one or more capture sequences shown in Table 7 (SEQ ID NOs: 373-436). In one aspect, the second set of non-cross-reactive oligonucleotide tags includes two or more oligonucleotide tags from a parent set shown in Table 19 (SEQ ID NOs: 1117-1180).

In one aspect, an eighth set of non-cross-reactive oligonucleotide tags is generated that is complementary to one or more capture sequences shown in Table 8 (SEQ ID NOs: 437-494). In one aspect, the second set of non-cross-reactive oligonucleotide tags includes two or more oligonucleotide tags from a parent set shown in Table 20 (SEQ ID NOs: 1181-1238).

In one aspect, a ninth set of non-cross-reactive oligonucleotide tags is generated that is complementary to one or more capture sequences shown in Table 9 (SEQ ID NOs: 495-558). In one aspect, the second set of non-cross-reactive oligonucleotide tags includes two or more oligonucleotide tags from a parent set shown in Table 21 (SEQ ID NOs: 1239-1302).

In one aspect, a tenth set of non-cross-reactive oligonucleotide tags is generated that is complementary to one or more capture sequences shown in Table 10 (SEQ ID NOs: 559-622). In one aspect, the second set of non-cross-reactive oligonucleotide tags includes two or more oligonucleotide tags from a parent set shown in Table 22 (SEQ ID NOs: 1303-1366).

In one aspect, an eleventh set of non-cross-reactive oligonucleotide tags is generated that is complementary to one or more capture sequences shown in Table 11 (SEQ ID NOs: 623-680). In one aspect, the second set of non-cross-reactive oligonucleotide tags includes two or more oligonucleotide tags from a parent set shown in Table 23 (SEQ ID NOs: 1367-1424).

In one aspect, a twelfth set of non-cross-reactive oligonucleotide tags is generated that is complementary to one or more capture sequences shown in Table 12 (SEQ ID NOs: 681-744). In one aspect, the second set of non-cross-reactive oligonucleotide tags includes two or more oligonucleotide tags from a parent set shown in Table 24 (SEQ ID NOs: 1425-1488).

In one aspect, the set of non-cross-reactive oligonucleotide tags includes one or more tags having a nucleotide sequence that is at least 95%, 96%, 97%, 98%, 99% or 100% identical to sequence that is complementary to a sequence of a capture oligonucleotide in Table 1 (SEQ ID NOs: 1-64), Table 2 (SEQ ID NOs: 65-122), Table 3 (SEQ ID NOs: 123-186), Table 4 (SEQ ID NOs: 187-250), Table 5 (SEQ ID NOs: 251-308), Table 6 (SEQ ID NOs: 309-372), Table 7 (SEQ ID NOs: 373-436), Table 8 (SEQ ID NOs: 437-494), Table 9 (SEQ ID NOs: 495-558), Table 10 (SEQ ID NOs: 559-622), Table 11 (SEQ ID NOs: 623-680), or Table 12 (SEQ ID NOs: 681-744). In one aspect, the set of non-cross-reactive oligonucleotide tags includes one or more tags having a nucleotide sequence that is at least 95%, 96%, 97%, 98%, 99% or 100% identical to a sequence shown in Table 13 (SEQ ID NOs: 745-808), Table 14 (SEQ ID NOs: 809-866), Table 15 (SEQ ID NOs: 867-930), Table 16 (SEQ ID NOs: 931-994), Table 17 (SEQ ID NOs: 995-1052), Table 18 (SEQ ID NOs: 1053-1116), Table 19 (SEQ ID NOs: 1117-1180), Table 20 (SEQ ID NOs: 1181-1238), Table 21 (SEQ ID NOs: 1239-1302), Table 22 (SEQ ID NOs: 1303-1366), Table 23 (SEQ ID NOs: 1367-1424), or Table 24 (SEQ ID NOs: 1425-1488).

In another aspect, the set of non-cross-reactive oligonucleotide tags includes one or more tags having a nucleotide sequence that includes at least 20, 21, 22, 23 or 24, consecutive nucleotides of a sequence that is complementary to a sequence of a capture oligonucleotide in Table 1 (SEQ ID NOs: 1-64), Table 2 (SEQ ID NOs: 65-122), Table 3 (SEQ ID NOs: 123-186), Table 4 (SEQ ID NOs: 187-250), Table 5 (SEQ ID NOs: 251-308), Table 6 (SEQ ID NOs: 309-372), Table 7 (SEQ ID NOs: 373-436), Table 8 (SEQ ID NOs: 437-494), Table 9 (SEQ ID NOs: 495-558), Table 10 (SEQ ID NOs: 559-622), Table 11 (SEQ ID NOs: 623-680), or Table 12 (SEQ ID NOs: 681-744). In another aspect, the set of non-cross-reactive oligonucleotide tags includes one or more tags having a nucleotide sequence that includes at least 20, 21, 22, 23 or 24, consecutive nucleotides of a sequence shown in Table 13 (SEQ ID NOs: 745-808), Table 14 (SEQ ID NOs: 809-866), Table 15 (SEQ ID NOs: 867-930), Table 16 (SEQ ID NOs: 931-994), Table 17 (SEQ ID NOs: 995-1052), Table 18 (SEQ ID NOs: 1053-1116), Table 19 (SEQ ID NOs: 1117-1180), Table 20 (SEQ ID NOs: 1181-1238), Table 21 (SEQ ID NOs: 1239-1302), Table 22 (SEQ ID NOs: 1303-1366), Table 23 (SEQ ID NOs: 1367-1424), or Table 24 (SEQ ID NOs: 1425-1488).

In another aspect, the set of non-cross-reactive oligonucleotide tags includes one or more tags having a nucleotide sequence that includes at least 20, 21, 22, 23 or 24, consecutive nucleotides of a sequence that is at least 95%, 96%, 97%, 98%, 99% or 100% identical to a sequence complementary to a sequence of a capture oligonucleotide in Table 1 (SEQ ID NOs: 1-64), Table 2 (SEQ ID NOs: 65-122), Table 3 (SEQ ID NOs: 123-186), Table 4 (SEQ ID NOs: 187-250), Table 5 (SEQ ID NOs: 251-308), Table 6 (SEQ ID NOs: 309-372), Table 7 (SEQ ID NOs: 373-436), Table 8 (SEQ ID NOs: 437-494), Table 9 (SEQ ID NOs: 495-558), Table 10 (SEQ ID NOs: 559-622), Table 11 (SEQ ID NOs: 623-680), or Table 12 (SEQ ID NOs: 681-744). In another aspect, the set of non-cross-reactive oligonucleotide tags includes one or more oligonucleotide tags having a nucleotide sequence that includes at least 20, 21, 22, 23 or 24, consecutive nucleotides of a sequence that is at least 95%, 96%, 97%, 98%, 99% or 100% identical to a sequence shown in Table 13 (SEQ ID NOs: 745-808), Table 14 (SEQ ID NOs: 809-866), Table 15 (SEQ ID NOs: 867-930), Table 16 (SEQ ID NOs: 931-994), Table 17 (SEQ ID NOs: 995-1052), Table 18 (SEQ ID NOs: 1053-1116), Table 19 (SEQ ID NOs: 1117-1180), Table 20 (SEQ ID NOs: 1181-1238), Table 21 (SEQ ID NOs: 1239-1302), Table 22 (SEQ ID NOs: 1303-1366), Table 23 (SEQ ID NOs: 1367-1424), or Table 24 (SEQ ID NOs: 1425-1488).

In one aspect, the non-cross-reactive oligonucleotide tags in the set are selected from: oligonucleotide tags having a sequence having at least 20, 21, 22, 23, or 24 consecutive nucleotides of a sequence selected from SEQ ID Nos: 745-808; oligonucleotide tags having a sequence having at least 95%, 96%, 97%, 98%, 99% or 100% identity to a sequence selected from SEQ ID Nos: 745-808; oligonucleotide tags having a sequence having at least 20, 21, 22, 23, or 24 consecutive nucleotides of a sequence having at least 95%, 96%, 97%, 98%, 99% or 100% identity to a sequence selected from SEQ ID Nos: 745-808; oligonucleotide tags having a sequence selected from SEQ ID Nos: 745-808; and combinations thereof.

In one aspect, the non-cross-reactive oligonucleotide tags in the set are selected from: oligonucleotide tags having a sequence having at least 20, 21, 22, 23 or 24 consecutive nucleotides of a sequence selected from SEQ ID Nos: 745-754; oligonucleotide tags having a sequence having at least 95%, 96%, 97%, 98%, 99% or 100% identity to a sequence selected from SEQ ID Nos: 745-754; oligonucleotide tags having a sequence having at least 20, 21, 22, 23 or 24 consecutive nucleotides of a sequence having at least 95%, 96%, 97%, 98%, 99% or 100% identity to a sequence selected from SEQ ID Nos: 745-754; oligonucleotide tags having a sequence selected from SEQ ID Nos: 745-754; and combinations thereof.

### J. Detection of Labeled Oligonucleotide Products

In one aspect, a method and kit are provided for labeling and detecting one or more target analytes in a sample. In one aspect, the presence of one or more target analytes in a sample is determined by generating a reaction product that includes an oligonucleotide tag. In one aspect, the reaction product includes a label. Various methods can be used to generate a reaction product. In one aspect, the reaction product is generated by methods described herein, including, but not limited to a sandwich assay, oligonucleotide ligation assay (OLA), primer extension assay (PEA), direct hybridization assay, polymerase chain reaction (PCR) based assay or other targeted amplification assay, and a nuclease protection assay.

### 1. Sandwich Assay

In one aspect, a method and kit are provided for detecting, identifying or quantifying one or more target analytes in a sample using a sandwich assay. In one aspect, the method or kit includes one or more sets of probes that includes a targeting probe and a detecting probe. In one aspect, the targeting probe includes a single stranded oligonucleotide tag that is complementary to at least a portion of a capture oligonucleotide immobilized on the support surface and a first binding partner. In one aspect, the first binding partner includes a first nucleic acid sequence. In one aspect, the first nucleic acid sequence of the first binding partner is complementary to a first region of a target nucleotide sequence in the sample. In one aspect, the first nucleic acid sequence of the first binding partner includes a therapeutic oligonucleotide. In one aspect, the therapeutic oligonucleotide includes an RNA oligonucleotide sequence. In one aspect, the therapeutic oligonucleotide is selected from miRNA, a therapeutic RNA, an mRNA, an RNA virus, an antisense oligonucleotide (ASO), or a combination thereof. In one aspect, the first nucleic acid sequence of the first binding partner is specifically bound by an anti-drug antibody (ADA) in a sample. In another aspect, the first binding partner includes an antibody that specifically binds to a target analyte in the sample. In one aspect, the detecting probe includes a label and a second binding partner.

In one aspect, the second binding partner includes a second nucleic acid sequence. In one aspect, the second nucleic acid sequence of the second binding partner is complementary to a second region of a target nucleotide sequence. In one aspect, the second nucleic acid sequence of the second binding partner includes a therapeutic oligonucleotide. In one aspect, the therapeutic oligonucleotide includes an RNA oligonucleotide sequence. In one aspect, the therapeutic oligonucleotide is selected from miRNA, a therapeutic RNA, an mRNA, an RNA virus, an antisense oligonucleotide (ASO), or a combination thereof. In one aspect, the second nucleic acid sequence of the second binding partner is specifically bound by an anti-drug antibody (ADA) in a sample. In one aspect, the first ASO of the first binding partner and the second ASO of the second binding partner are specifically bound by the same anti-drug antibody.

In one aspect, the nucleotide sequence of the first ASO of the first binding partner and the nucleotide sequence of the second ASO of the second binding partner are at least about 95%, 96%, 97%, 98%, 99% or 100% identical. In one aspect, the second binding partner includes an antibody that specifically binds to a target analyte in the sample. In one aspect, the targeting probe and the detecting probe can bind concurrently to the same target analyte in the sample to form a reaction product. In one aspect, the reaction product is a sandwich complex.

In one aspect, the method or kit include a plurality of sets of probes that can be used in a multiplexed array to detect, identify, or quantify a plurality of target analytes in parallel. In one aspect, each set of probes includes a targeting probe with a first binding partner that specifically binds to a different first target analyte than the targeting probe in another set and an oligonucleotide tag having a sequence that is complementary to a different capture oligonucleotide sequence than the targeting probes in the other sets. In one aspect, each set of probes includes a detecting probe that includes a second binding partner that specifically binds the first target analyte and a label. In one aspect, the method or kit include a plurality of sets of oligonucleotide probes. In one aspect, each set of probes includes a targeting probe in which the first binding partner includes a nucleic acid sequence that is complementary to a first target nucleotide sequence and an oligonucleotide tag having a sequence that is complementary to a capture oligonucleotide sequence, wherein the target nucleotide sequence for the targeting probe in one set is different than the target nucleotide sequence in a targeting probe in another set. In one aspect, the sequence of the oligonucleotide tag of the targeting probe in one set is complementary to a different capture oligonucleotide sequence than the sequence of the oligonucleotide tag of targeting probes in another set. In one aspect, the method or kit includes a detecting probe that has a second binding partner that is a second target nucleotide sequence complementary to a second target nucleotide sequence in the one or more target nucleotides.

In one aspect, the method includes a step of providing an array that includes one or more carbon-based electrodes having one or more surfaces; and one or more non-cross-reactive capture oligonucleotides described herein, wherein one or more non-cross-reactive capture oligonucleotides are immobilized in one or more binding domains on one or more surfaces of the one or more carbon-based electrodes. In one aspect, the method includes a step of associating one or more target analytes with an oligonucleotide tag that is complementary to at least a portion of a capture oligonucleotide immobilized on the support surface and a label and then contacting the array with the composition that includes one or more tagged and labeled target analytes or reaction products. As used herein, “associating” or “associated” means that the oligonucleotide tag or label are either covalently or noncovalently bound to the target analyte. In one aspect, one or more target analytes are associated with an oligonucleotide tag and a label in a sandwich complex. In one aspect, the target analyte is used to generate a reaction product that includes an oligonucleotide tag and a label. In one aspect, the method includes a step of incubating the sandwich complex or reaction products with a support surface under conditions in which the oligonucleotide tags of the sandwich complex or reaction product hybridize to their corresponding complementary capture oligonucleotides and identifying, detecting or quantifying the target analyte based on the presence or absence of the label in an array location.

### 2. Oligonucleotide Ligation Assay (OLA)

In one aspect, the array is contacted with a composition that includes a plurality of target analytes, wherein each target analyte is associated with an oligonucleotide tag that is complementary to a different capture oligonucleotide and the target analyte can be identified, detected or quantified based on the binding of the oligonucleotide tag in an array location. In one aspect, the array is contacted with a composition that includes a plurality of tagged and labeled reaction products, wherein each target analyte is used to generate a reaction product that includes an oligonucleotide tag that is complementary to a different capture oligonucleotide and the target analyte in the sample can be identified, detected or quantified based on the binding of the reaction product in an array location.

In one aspect, a tagged and labeled reaction product is prepared by an oligonucleotide ligation assay (OLA) and can be captured and detected to identify, detect or quantify one or more target nucleotide sequences. In one aspect, the ligation assay is used to detect, identify or quantify a single nucleotide polymorphism (SNP) in one or more target nucleotide sequences. In one aspect, the ligation assay is performed following amplification of one or more target nucleotide sequences in a sample. In another aspect, the ligation assay is performed on a sample in which one or more target nucleotide sequences have not been amplified. In one aspect, the reaction product from the ligation assay is amplified before capture and detection. In another aspect, the reaction product from the ligation assay is not amplified before capture and detection. The reaction product of the ligation assay can be amplified using known methods.

Methods for performing oligonucleotide ligation reactions are known and generally include the following steps: A sample that contains or may contain one or more nucleotide sequences of interest is contacted with pairs of single stranded oligonucleotide probes that are complementary to one or more target nucleotide sequences and are allowed to hybridize to the target nucleotide sequence. Probes that hybridize to adjacent regions of the target nucleotides sequences are ligated to form a reaction product. In one aspect, these steps can be repeated to obtain multiple copies of the reaction product. In one aspect, the nucleotide sequences in the ligation reaction mixture are denatured before the annealing step. The target nucleotide sequence can be detected, identified or quantified based on the presence, absence or quantity of the reaction product in the sample.

The joining of probes by DNA ligase is dependent on three events: (1) the oligonucleotide probes must hybridize to complementary sequences within the target nucleotide sequence; (2) the oligonucleotide probes must be adjacent to one another in a 5′- to 3′-orientation with no intervening nucleotides; and (3) the oligonucleotide probes must have perfect base-pair complementarity with the target nucleotide sequence at the site of their join. A single nucleotide mismatch between the primers and target may inhibit ligation.

In one aspect, the probes are generated by identifying a nucleic acid sequence that includes about 40 base pairs on both sides of a SNP site in a target nucleotide sequence (for a total of about 80 base pairs) and creating a probe having complementary sequences upstream and downstream of the SNP that span about 18 to about 28 nucleotides. In one aspect, two targeting probes are generated that differ at the SNP position. Typically, only one detecting probe is needed to detect the wild type and variant alleles.

In a further aspect, the target nucleotide sequence is a small nucleic acid, e.g., at least about 15 base pairs, at least about 16 base pairs, at least about 17 base pairs, at least about 18 base pairs, at least about 19 base pairs, or at least about 20 base pairs and up to about 20 base pairs in length, up to about 25 base pairs in length, up to about 30 base pairs in length, up to about 40 base pairs in length or up to about 50 base pairs in length. In one aspect, the probe for detecting such small nucleic acid targets includes at least about 8 base pairs, at least about 9 base pairs, at least about 10 base pairs, at least about 11 base pairs, or at least about 12 base pairs and up to about 20 base pairs in length, up to about 25 base pairs in length, up to about 30 base pairs in length, up to about 40 base pairs in length or up to about 50 base pairs in length, and the probe and the small nucleic acid target are ligated after hybridizing another as described herein.

The length of the oligonucleotide probe sequences can vary based on the ligation temperature requirements for the OLA reaction (e.g., between about 62° C. and about 64° C.). Bases can be added or removed from the targeting or detecting probes until the probe length is suitable for a given reaction temperature.

After the sequence and length of the targeting and detecting probes is determined, an oligonucleotide tag can be added to the targeting probe. In one aspect, the oligonucleotide tag is added to the 5′ end of an upstream targeting probe. In one aspect, each oligonucleotide tag is complementary to a different capture oligonucleotide immobilized on the support surface. In one aspect, the detecting probe includes a label. In one aspect, the detecting probe includes a 5′ phosphate group and 3′ label. In one aspect, the detecting probe includes a 5′ phosphate group and a 3′ biotin label.

In one aspect, the method includes the use of more than one pair of probes. In one aspect, a pair of probes is provided for each target sequence in a sample. In one aspect, three probes are prepared for the detection of a SNP pair, two targeting probes that vary at the single nucleotide polymorphism and one detecting probe. In one aspect, the two targeting probes include a 5′ oligonucleotide tag and a 3′ nucleic acid that is complementary to either the wild type or variant single nucleotide polymorphism in a target nucleic acid of interest and the detecting probe includes a 3′ label. In one aspect, the 3′ label is a primary binding reagent that binds to a detectable secondary binding reagent. In one aspect, the 3′ label includes biotin and the secondary binding reagent includes MSD SULFO-TAG streptavidin. In one aspect, a pair of probes is prepared for each allele at a polymorphic site, for example, two probes may be prepared, one for the wild type allele and one for the mutant allele. In one aspect, a ligation reaction is performed for each target nucleotide sequence. In another aspect, a multiplexed ligation reaction is performed for more than one target nucleotide sequence. In one aspect, a multiplexed ligation reaction is performed for between about 1 and about 100, or up to 2, 3, 4, 5, 6, 7, 8, 9, 10, 25, 50, 75, or 100 target nucleotide sequences. In one aspect, the multiplexed ligation reaction is performed to detect, identify or quantify up to 10 target nucleotide sequences in each well. In one aspect, a plurality of allele pairs are detected, identified or quantified. In one aspect, up to five allele pairs (i.e., wild type and mutant SNP pairs) are detected, identified or quantified in each well. In one aspect, detecting, identifying or quantifying includes determining whether a sample is homozygous, heterozygous or null for a variant allele.

In one aspect, the probes are joined using a template-dependent ligase, for example, a DNA ligase such as E. coli DNA ligase, T4 DNA ligase, T. aquaticus (Taq) ligase, T. Thermophilus DNA ligase, or Pyrococcus DNA ligase. In one aspect, the ligase is a thermostable ligase. In another aspect, the probes are joined by chemical ligation. In one aspect, hybridization and ligation are performed in a combined step, for example, using multiple thermocycles and a thermostable ligase. In one aspect, the reaction mixture includes at least about 100 U/mL, 500 U/mL or 1000 U/mL and up to about 1500 U/mL or 2000 U/mL ligase.

In one aspect, the ligation assay is performed by combining the sample with one or more pairs of probes and a ligase in a ligation buffer. In one aspect, the sample, probes and ligase are combined with ligation buffer to form a ligation reaction mixture having a volume of at least about 10 µL, 15 µL or 20 µL and up to about 20 µL, 25 µL or 50 µL.

In one aspect, each pair of probes includes a targeting probe and a detecting probe. In one aspect, the targeting probe includes a nucleotide sequence that is complementary to a first region of a target nucleotide sequence and a single stranded oligonucleotide tag that is complementary to at least a portion of a capture oligonucleotide immobilized on a support surface. In one aspect, the detecting probe includes a label and a nucleotide sequence that is complementary to a second region of the target nucleotide sequence that is adjacent to the first region to which the first nucleic acid sequence of the targeting probe sequence is complementary. In one aspect, the 5′-end of the targeting probe is phosphorylated and is adjacent to the 3′-hydroxyl of the detecting probe when the pair of probes is annealed to the target nucleotide sequence, such that the ends of the two probes may be ligated by the formation of a phosphodiester bond. In one aspect, the 5′-end of the detecting probe is phosphorylated and is adjacent to the 3′-hydroxyl of the targeting probe when the pair of probes is annealed to the target nucleotide sequence, such that the ends of the two probes may be ligated by the formation of a phosphodiester bond.

In one aspect, the targeting probe includes between about 5 and about 100, about 10 and about 50, about 20 and about 30, or at least about 5, 6, 7, 8, 9, 10, 15, 20 or 25 and up to about 30, 35, 40, 45, 50, 75 or 100 nucleotides. In one aspect, at least about 1 nM, 2 nM, 3 nM, 4 nM or 5 nM and up to about 5 nM, 10 nM, 25 nM or 50 nM of the targeting probe is included in the reaction mixture.

In one aspect, the entire length of the targeting probe is complementary to a target nucleotide sequence. In another aspect, a portion of the targeting probe is complementary to the target nucleotide sequence. In one aspect, the targeting probe is complementary to the target nucleotide sequence downstream of a polymorphic site. In one aspect, the targeting probe is an allele-specific probe that includes a nucleic acid sequence that is complementary to a region of a target nucleotide sequence that includes a single nucleotide variant. In one aspect, the targeting probe is an allele-specific probe that includes a nucleic acid sequence that is complementary to a region of a target nucleotide sequence that includes a single nucleotide polymorphism. In one aspect, a 3′-terminal nucleic acid of the targeting probe is complementary to a polymorphic nucleic acid of the target nucleotide sequence. In another aspect, a 3′-terminal nucleic acid of the targeting probe is complementary to a nucleotide 3′ of the polymorphic nucleic acid of the target nucleotide sequence.

In one aspect, the targeting probe includes a tag that specifically binds to a capture molecule. In one aspect, the tag includes a single stranded oligonucleotide sequence that is complementary to at least a portion of the nucleotides sequence of a single stranded capture oligonucleotide. In one aspect, the tag is attached to the 5′-end of the targeting probe. In another aspect, the tag is attached to the 3′-end of the targeting probe. In one aspect, the tag is not complementary to and does not hybridize with the target nucleotide sequence.

In one aspect, the tag includes a nucleotide sequence that is at least about 1, 2, 3, 4, 5, 6, 7, 8, 9, or 10 and up to about 11, 12, 13, 14, 15, 16, 17, 18, 19, or 20, or between about 1 and about 20 or between about 10 and about 15 or between about 12 and about 13 nucleotides shorter than the complementary capture oligonucleotide sequence. In one aspect, the tag has a nucleotide sequence that is at least about 15, 16, 17, 18, 19, 20, 21, 22, 23, 24 or 25 and up to about 25, 26, 27, 28, 29, 30, 31, 32, 33, 34, 35, 36, 37, 38, 39 or 40, or between about 15 and about 40, or about 20 and about 30 nucleotides in length.

In one aspect, the tag includes a nucleotide sequence that is complementary to at least a portion of a nucleotide sequence for a capture oligonucleotide shown in SEQ ID Nos: 1-10. In one aspect, the tag includes a nucleotide sequence that is complementary to between about 20 and about 25, or about 24 consecutive nucleotides of a sequence of a capture oligonucleotide shown in SEQ ID Nos: 1-10. In one aspect, the single stranded oligonucleotide tag is prepared using known methods based on the sequence of the capture oligonucleotide.

In one aspect, each pair of oligonucleotide probes includes a detecting probe having between about 5 and about 100, about 10 and about 50, about 20 and about 30, or at least about 5, 6, 7, 8, 9, 10, 15, 20 or 25 and up to about 30, 35, 40, 45, 50, 75 or 100 nucleotides.

In one aspect, the targeting and detecting probes have a melting temperature of between about 60° C. and about 65° C., or between about 62° C. and about 64° C. In one aspect the targeting and detecting probes have similar melting temperatures (i.e., within about 1° C., 2° C., 3° C., 4° C., or 5° C.).

In one aspect, the targeting and detecting probes for a target nucleotide sequence are included in the ligation reaction mixture in a 1:1 ratio. In another aspect, the detecting probe is included in excess, for example, the ligation reaction mixture can include at least about 5×, 10× or 20× more of the detecting probe as compared to the targeting probe. In one aspect, at least about 10 nM, 25 nM, 50 nM, 75 nM, 100 nM, 150 nM or 200 nM of the detecting probe is included in the reaction mixture.

In one aspect, the entire length of the detecting probe is complementary to the target nucleotide sequence. In another aspect, a portion of the detecting probe is complementary to the target nucleotide sequence. In one aspect, the detecting probe is complementary to the target nucleotide sequence upstream of a polymorphic site. In one aspect, the detecting probe includes a nucleic acid sequence that is complementary to a region of a target nucleotide sequence that includes a single nucleotide variant. In one aspect, the detecting probe includes a nucleic acid sequence that is complementary to a region of a target nucleotide sequence that includes a single nucleotide polymorphism. In one aspect, a 5′-terminal nucleic acid of the detecting probe is complementary to a polymorphic nucleic acid of the target nucleotide sequence. In another aspect, a 5′-terminal nucleic acid of the detecting probe hybridizes to a nucleic acid that is 5′ of a polymorphic nucleic acid of the target nucleotide sequence.

In one aspect, the detecting probe includes a label. In one aspect, the label is attached to the 3′ end of the detecting probe. In one aspect, the label is attached to the 3′ end of the detecting probe and the 5′ end has a nucleic acid sequence that is complementary to a sequence of the target nucleotide immediately adjacent to a sequence of the target nucleotide to which the 3′ end of the targeting probe hybridizes. In one aspect, the label is attached to the 5′ end of the detecting probe and the 3′ end has a nucleic acid sequence that is complementary to a sequence of the target nucleotide immediately adjacent to a sequence of the target nucleotide to which the 5′ end of the targeting probe hybridizes.

In one aspect, the targeting probe hybridizes to the target nucleotide sequence such that the 3′ end of the targeting probe is situated directly over a polymorphic nucleotide of the target nucleotide sequence and the detecting probe hybridizes to the target nucleotide sequence adjacent to the polymorphic site, providing a 5′ end for the ligation reaction. If the targeting probe is complementary to the polymorphic nucleotide in the target nucleotide sequence, the first oligonucleotide will hybridize to the target nucleotide sequence at the polymorphic site and ligation can occur. If the targeting probe is not complementary to the polymorphic nucleotide in the nucleotide sequence, the first oligonucleotide will not hybridize to the target nucleotide sequence at the polymorphic site and ligation will not occur.

In another aspect, the targeting probe hybridizes to the target nucleotide sequence such that the terminal 5′- base of the targeting probe is situated directly over a polymorphic nucleotide of the target nucleotide sequence and the detecting probe hybridizes to the target nucleotide sequence adjacent to the polymorphic site, providing a 3′ end for the ligation reaction.

In another aspect, the detecting probe hybridizes to the target nucleotide sequence such that the terminal 5′- base of the detecting probe is situated directly over a polymorphic nucleotide of the target nucleotide sequence and the targeting probe hybridizes to the target nucleotide sequence adjacent to the polymorphic site, providing a 3′ end for the ligation reaction.

In another aspect, the detecting probe hybridizes to the target nucleotide sequence such that the terminal 3′- base of the detecting probe is situated directly over a polymorphic nucleotide of the target nucleotide sequence and the targeting probe hybridizes to the target nucleotide sequence adjacent to the polymorphic site, providing a 5′ end for the ligation reaction.

In one aspect, the method includes (i) contacting a sample containing one or more target nucleotides with a pair of oligonucleotide probes and a DNA ligase to form a ligation reaction mixture; (ii) hybridizing the pair of probes to the target nucleotide sequence, wherein the pair includes a capture or detecting probe with a terminal 3′ or 5′ base that is situated directly over a polymorphic nucleotide of the target nucleotide sequence; (iii) ligating the targeting and detecting probes together to form a labeled and tagged reaction product; (iv) contacting a support surface on which one or more capture oligonucleotides are immobilized with the labeled and tagged reaction product; (v) allowing the tag to hybridize to the capture oligonucleotide; and (vi) detecting the presence of the tagged and labeled reaction product.

In one aspect, the probes used in the ligation assay are included at in excess over the target nucleotide sequence (i.e., at the nM level) and, therefore, in some cases non-specific binding of oligonucleotides and target can be detected on plate as a positive signal. While not wishing to be bound by theory, it is believed that non-specific hybridization can be the result of the probes hybridizing to the target nucleotide sequence and remaining hybridized without ligation, which results in a signal that is not due to a ligation reaction product, but a non-specific signal referred to as bridging background.

In one aspect, the method includes providing one or more blocking probes in the ligation reaction mixture. In one aspect, including one or more blocking probes in the ligation reaction mixture reduces non-specific bridging background. As used herein, the term “blocking probe” refers to a single stranded nucleotide sequence that is complementary to the target nucleotide sequence and straddles the probe ligation site but does not include a tag or label, or a single stranded nucleotide sequence that is complementary to a probe designed to hybridize to the target nucleotide sequence. In one aspect, the blocking probe is largely colinear with the probe sequences. In one aspect, the blocking probe includes at least about 20, 25, 30, 35, 40, 45 or 50 and up to about 50, 75, 100, 150, or 200, or between about 20 and about 200, or between about 50 and about 100 nucleotides that are complementary to either the target nucleotide sequence or a probe directed against the target nucleotide sequence. In one aspect, a pair of blocking probes is included in the ligation reaction mixture, in which the first blocking probe has a sequence identical to the connection probe, but without the complementary oligonucleotide tag; and the second blocking probe has a sequence identical to the detecting probe, but without the biotin label. In one aspect, up to 2, 3, 4 or 5 additional nucleotides can be added to the 5′- and 3′-end of the blocking probe that are complementary to the target nucleotide sequence adjacent to the probe sequences.

While not wishing to be bound by theory, it is believed that the presence of a blocking probe can reduce formation of complexes in which the target nucleotide sequence functions as a bridge for probes that are annealed to the target sequence, but not ligated, such that the complex can generate a false signal. In one aspect, a pair of blocking probe is included in the ligation reaction mixture. In another aspect, one or more blocking probes are included in the ligation reaction mixture in excess over the corresponding OLA probes. In one aspect, one or more blocking probes are included in at least about 10×, 20×, 30×, 40×, 50×, 60×, 70×, 80×, 90× or 100× molar excess over the corresponding OLA probes.

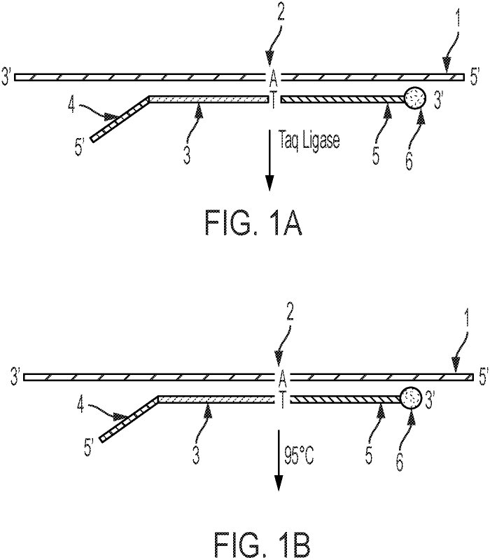

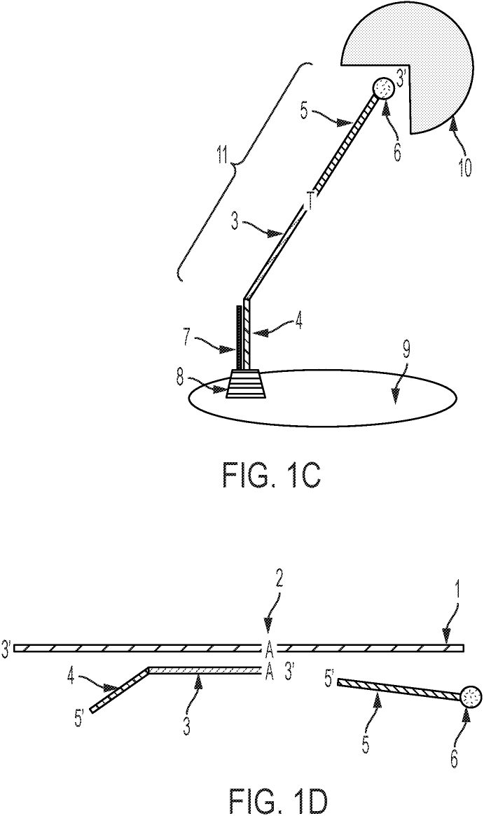

One embodiment of an oligonucleotide ligation assay is represented schematically in FIGS. 1A 1B. Briefly, a target nucleotide sequence 1 that includes a polymorphic site 2 is contacted with a pair of oligonucleotide probes that includes a targeting probe 3 with a oligonucleotide tag 4 and a nucleotide that is complementary to the polymorphic site and a detecting probe 5 with a label 6. The oligonucleotide probes 3, 5 are allowed to hybridize to the target nucleotide sequence. (FIG. 1A) Oligonucleotide probes 3, 5 that hybridize with perfect complementarity at the polymorphic site are ligated to form a tagged 4 and labeled 6 reaction product 11. (FIG. 1B) The reaction mixture containing the tagged 4 and labeled 6 ligation product 11 is introduced onto a support surface having one or more capture oligonucleotides 7 immobilized in one or more binding domains 9. A signal 10 is detected if the tagged 4 and labeled 6 ligation product 11 is immobilized on the support surface through hybridization between complementary nucleotide sequenced contained in the tagged oligonucleotide 4 and the capture oligonucleotide 7. (FIG. 1C).

In one aspect, a multiplex ligase detection reaction is provided. In one aspect, a sample is contacted with one or more allele-specific probes and one or more common probes. In one aspect, one or more allele-specific probes include an upstream probe that includes 5′ oligonucleotide tag with a sequence that is complementary to a capture oligonucleotide sequence and a 3′ sequence that corresponds to a polymorphism of interest. In one aspect, one or more common probes is a downstream probe that is 5′-phosphorylated and 3′-biotinylated. In one aspect, the multiplex ligation probes are contacted with a sample containing one or more target analytes, allowed to hybridize and adjacent probes are ligated with a DNA ligase to form a ligation product. In one aspect, one or more immobilized capture oligonucleotides are contacted with the ligation products and the oligonucleotide tags are allowed to hybridize with their corresponding capture oligonucleotides. The immobilized ligation products can be detected, for example, using labeled streptavidin, for example, SULFO-TAG labeled streptavidin.

In one aspect, an oligonucleotide ligation assay (OLA) is used for detection, identification, and/or quantification of a target nucleotide sequence that is contained in a sample that may contain degradation products of the target nucleotide sequence, also referred to as oligonucleotide metabolites. In one aspect, the sample containing the target nucleotide sequence further includes one or more oligonucleotide metabolites. In one aspect, an OLA is used to measure the amount of target nucleotide sequence in a sample relative to oligonucleotide metabolites. In one aspect, an OLA is used to determine a pharmacokinetic parameter of a target nucleotide sequence. In one aspect, the pharmacokinetic parameter measured is clearance, volume distribution, plasma concentration, half-life, peak time, peak concentration, rate of availability, or combination thereof. Measurement and interpretation of pharmacokinetic parameters are described herein. In one aspect, the target nucleotide sequence is a therapeutic oligonucleotide. In one aspect, the therapeutic oligonucleotide is an antisense oligonucleotide (ASO). In one aspect, the oligonucleotide metabolite is a therapeutic oligonucleotide metabolite. Therapeutic oligonucleotides, ASOs, and their metabolism and pharmacology are described herein.

In one aspect, the oligonucleotide metabolite is shorter than the target nucleotide sequence by 1 or more nucleotides, 2 or more nucleotides, 3 or more nucleotides, 4 or more nucleotides, 5 or more nucleotides, 6 or more nucleotides, 7 or more nucleotides, 8 or more nucleotides, 9 or more nucleotides, 10 or more nucleotides, 15 or more nucleotides, or 20 or more nucleotides. In one aspect, the oligonucleotide metabolite is shorter than the target nucleotide sequence by about 1%, about 2%, about 3%, about 4%, about 5%, about 6%, about 7%, about 8%, about 9%, or about 10%. In one aspect, the target nucleotide sequence is a therapeutic oligonucleotide. In one aspect, the therapeutic oligonucleotide is an antisense oligonucleotide (ASO). In one aspect, the oligonucleotide metabolite is a therapeutic oligonucleotide metabolite.

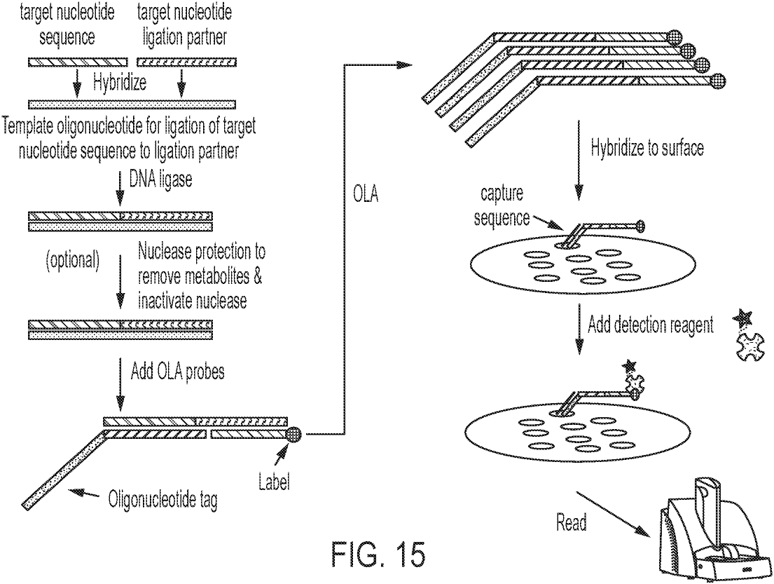

An exemplary embodiment is illustrated in FIG. 15. In FIG. 15, a sample containing a target nucleotide sequence is contacted with a template oligonucleotide. The template oligonucleotide comprises a first sequence complementary to the target nucleotide sequence, and a second sequence adjacent to the first sequence and complementary to a ligation partner of the target nucleotide sequence. In one aspect, the target nucleotide sequence hybridizes to the first sequence of the template oligonucleotide, and the ligation partner of the target nucleotide sequence hybridizes to the second sequence of the template oligonucleotide. In one aspect, the target nucleotide sequence and ligation partner hybridize over the entire length of the template oligonucleotide. In one aspect, the target nucleotide sequence and ligation partner hybridize with the template oligonucleotide to form a double-stranded complex. The target nucleotide sequence and ligation partner are ligated together using methods described herein to form an target nucleotide sequence ligation product. The target nucleotide sequence ligation product is then contacted with pairs of single stranded oligonucleotide probes that are complementary to the target nucleotide sequence ligation product and allowed to hybridize to the target nucleotide sequence ligation product. In one aspect, probes capable of hybridizing to adjacent regions of the target nucleotide sequence ligation product are added to the target nucleotide sequence ligation product. In one aspect, two adjacent probes, each hybridizing to adjacent regions of the target nucleotide sequence ligation product, are ligated to form a reaction product. In one aspect, the probes comprise a targeting probe and a detecting probe as described herein. In one aspect, the targeting probe and detecting probe hybridize over the entire length of the target nucleotide sequence ligation product. In one aspect, the targeting probe comprises a oligonucleotide tag. Targeting probes and oligonucleotide tags are further described herein. In one aspect, the oligonucleotide tag is complementary to at least a portion of a capture oligonucleotide immobilized on a support surface. In one aspect, the detecting probe comprises a label. Detecting probes and labels are further described herein. In one aspect, the label comprises biotin, and the detection reagent is linked to streptavidin. In another aspect, the label comprises a hapten, and the detection reagent is linked to a hapten binding partner such as an antibody. Labels, detection reagents, and modes of binding between labels and detection reagents are further described herein. In one aspect, the surface is contacted with a detection reagent for binding to the label. In one aspect, the detection reagent is an electrochemiluminescent reagent. In one aspect, the detection reagent comprises an MSD SULFO-TAG. In one aspect, electrochemiluminescence is measured as described herein to detect, identify, and/or quantify the target nucleotide sequence. In one aspect, the amount of target nucleotide sequence in the sample is measured to determine a pharmacokinetic parameter of the target nucleotide sequence. In one aspect, the target nucleotide sequence is a therapeutic oligonucleotide. In one aspect, the therapeutic oligonucleotide is an antisense oligonucleotide. In one aspect, the therapeutic oligonucleotide is detected without amplifying the therapeutic oligonucleotide. In one aspect, the therapeutic oligonucleotide in the sample is detected without a nucleic acid extraction step.

In one aspect, the sample containing the target nucleotide sequence also includes one or more oligonucleotide metabolites. In one aspect, the oligonucleotide metabolite interferes with the detection, identification, and/or quantification of the target nucleotide sequence. Thus, it may be desirable to remove oligonucleotide metabolites from the sample. Accordingly, in one aspect, a nuclease specific for single-stranded oligonucleotides (i.e., a “single-strand-specific nuclease”) is added to the sample while the target nucleotide sequence ligation product and the template oligonucleotide are hybridized and prior to addition of the probes, as outlined in FIG. 15. The single-strand-specific nuclease specifically removes single-stranded oligonucleotide metabolites while being substantially unreactive to the hybridized target nucleotide sequence ligation product and template oligonucleotide. In one aspect, the single-strand-specific nuclease additionally removes excess unhybridized template oligonucleotide. Non-limiting examples of single-strand-specific nucleases include nuclease S1 (e.g., isolated from Aspergillus oryzae), nuclease P1 (e.g., isolated from Penicillium citrinum), nuclease MB (e.g., isolated from mung bean Vigna radiata), and nucleases isolated from Alteromonas espejiana, Neurospora crassa, and Ustilago maydis. Single-strand-specific nucleases can also include, e.g., RNases such as RNase A, RNase H, RNase I, RNase III, RNase L, RNase P, RNase PhyM, RNase T1, RNase T2, RNase U2, RNase V, PNPase, RNase PH, RNase R, RNase D, RNase T, RNaseONE, oligoribonuclease, exoribonuclease I, and exoribonuclease II. Additional nucleases that may be suitable for the present methods include certain DNases. Additional nucleases, including single-strand-specific nucleases, are provided in, e.g., Yang, Q Rev Biophys 44(1):1-93 (2011) and Desai et al., FEMS Microbiol Rev 26:457-491 (2003). In one aspect, the target nucleotide sequence is a therapeutic oligonucleotide. In one aspect, the therapeutic oligonucleotide is an antisense oligonucleotide. In one aspect, the oligonucleotide metabolite is a therapeutic oligonucleotide metabolite. In one aspect, the target nucleotide sequence includes RNA. In one aspect, the target nucleotide sequence includes an miRNA, a therapeutic RNA, an mRNA, an RNA virus, or a combination thereof.

### 3. Primer Extension Assay (PEA)

In another aspect, one or more target nucleotide sequences in a sample is detected, identified or quantified using a primer extension assay (PEA). In one aspect, the target nucleotide sequence includes one or more single nucleotide variants (SNV). In another aspect, the nucleotide sequence includes one or more single nucleotide polymorphisms (SNP). In one aspect, primer extension is performed following amplification of the target nucleotide sequence in a sample. In another aspect, primer extension is performed on a sample that has not been amplified.

Methods for performing primer extension assays are known and generally include the following steps: A sample is contacted with a probe having a nucleotide sequence complementary to a target nucleotide sequence. In one aspect, the entire length of the probe is complementary to a target nucleotide sequence. In another aspect, a portion of the probe is complementary to the target nucleotide sequence. In one aspect, the probe includes a nucleic acid sequence that is complementary to the nucleic acid sequence of the target nucleotide sequence immediately flanking the 3′ end of a polymorphism, such that the probe hybridizes to the target nucleotides sequence downstream of the polymorphic nucleotide. In one aspect, the probe includes between about 5 and about 100, about 10 and about 50, about 20 and about 30, or at least about 5, 6, 7, 8, 9, 10, 15, 20 or 25 and up to about 30, 35, 40, 45, 50, 75 or 100 nucleotides.

In one aspect, the probe is a targeting probe that includes a tag that specifically binds to a capture molecule. In one aspect, the tag includes a single stranded oligonucleotide sequence that is complementary to a nucleotides sequence of a single stranded capture oligonucleotide. In one aspect, the tag is attached to the 5′ end of the targeting probe. In one aspect, the tag is attached to the 5′ end of the targeting probe and a 3′ terminal nucleic acid of the targeting probe is complementary to a nucleic acid immediately downstream of a polymorphic site of the target nucleotide sequence. In one aspect, the single stranded oligonucleotide tag is prepared using known methods by the end user based on the sequence of the capture oligonucleotide.

In one aspect, the tag includes a nucleotide sequence that is at least about 1, 2, 3, 4, 5, 6, 7, 8, 9, or 10 and up to about 11, 12, 13, 14, 15, 16, 17, 18, 19, or 20, or between about 1 and about 20 or between about 10 and about 15 nucleotides shorter than the capture oligonucleotide sequence. In one aspect, the tag has a nucleotide sequence that is at least about 15, 16, 17, 18, 19, 20, 21, 22, 23, 24 or 25 and up to about 25, 26, 27, 28, 29, 30, 31, 32, 33, 34, 35, 36, 37, 38, 39 or 40, or between about 15 and about 40, or about 20 and about 30 nucleotides in length. In one aspect, the tag includes a nucleotide sequence that is complementary to at least a portion of a nucleotide sequence for a capture oligonucleotide shown in SEQ ID NOs: 1-64. In one aspect, the tag includes a nucleotide sequence that is complementary to at least a portion of a nucleotide sequence for a capture oligonucleotide shown in SEQ ID NOs: 1-10.

In one aspect, one or more tag oligonucleotides contain a sequence that is complementary to full sequence of their corresponding capture oligonucleotide. In one aspect, one or more tag oligonucleotides contain a sequence that is complementary to only a portion of the sequence of their corresponding capture oligonucleotide. For example, and not by way of limitation, the capture oligonucleotide may contain a linker as described herein, which may consist of or comprise an oligonucleotide sequence that is not complementary to the tag oligonucleotide sequence, proximal to the surface to which it is attached (e.g., beginning with a thiol-modified terminal nucleotide). The region of complementarity between the tag and capture oligonucleotides may also vary in length. In some aspects of the invention the regions of complementarity between the oligonucleotides is at least 20, 21, 22, 23, 24, 25, 26, 27, 28, 29, 30, 31, 32, 33, 34, 35 or 36 nucleotides in length.

In one aspect, the method includes contacting a sample containing one or more target nucleotide sequences with a targeting probe and hybridizing the targeting probe to the target oligonucleotide in the presence of a primer extension reaction mixture that includes a polymerase and one or more 2′3′-dideoxynucleotide triphosphates (ddNTPs), including, for example, ddA, ddT, ddC, ddG. In one aspect, ddNTP is complementary to the polymorphic site is labeled. In one aspect, ddNTP that are not complementary to the polymorphic site are not labeled. In one aspect, the ddNTP that is complementary to a wild-type polymorphic nucleotide is labeled. In another aspect, ddNTP is complementary to a mutant polymorphic nucleotide is labeled. In one aspect, the 3′ end of the targeting probe is extended by a single ddNTP. In one aspect, the primer is extended by one labeled ddNTP to form a tagged and labeled reaction product when the labeled ddNTP is complementary to the polymorphic nucleotide. When the labeled ddNTP is not complementary to the polymorphic nucleotide such that the primer is extended by unlabeled ddNTP and is not detected.

Suitable polymerase enzymes include, but are not limited to, DNA polymerase, RNA polymerase, DNA dependent RNA polymerase (reverse transcriptase) and active subunits thereof, including, for example, the Klenow fragment of DNA polymerase. In one aspect, the polymerase is DNA polymerase. In one aspect, the polymerase is a thermostable polymerase such as a Taq polymerase.

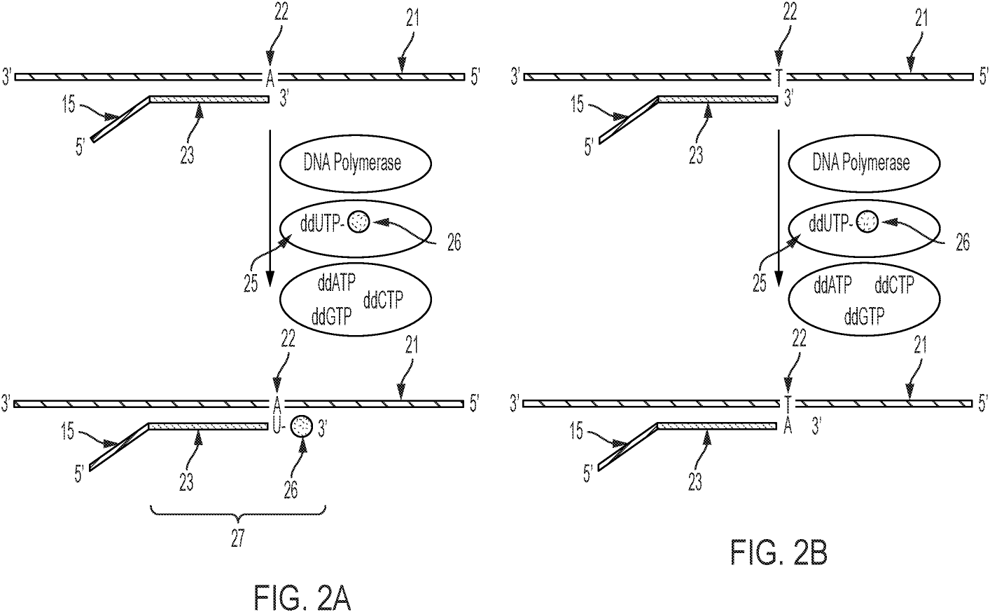

One embodiment of a primer extension assay is represented schematically in FIGS. 2A 2B. Briefly, a target nucleotide sequence 21 that includes a polymorphic site 22 is contacted with a targeting probe 23 with a oligonucleotide tag 25 in the presence of a primer extension reaction mixture that includes DNA polymerase and 2′3′-dideoxynucleotide triphosphates (ddNTPs), i.e., ddA, ddT, ddC, ddG, wherein the ddNTP 25 that is complementary to the polymorphic site is labeled 26. The 3′ end of the targeting probe is extended by a single ddNTP. As shown in FIG. 2A, the primer is extended by one labeled ddNTP to form a tagged and labeled reaction product when the labeled ddNTP is complementary to the polymorphic nucleotide. As shown in FIG. 2B, the primer is extended by an unlabeled ddNTP when the polymorphic nucleotide is not complementary to labeled ddNTP, resulting in an unlabeled reaction product that will not be detected.

### 4. Direct Hybridization

In one aspect, a method or kit is provided for detecting, identifying or quantifying one or more target analytes in a sample using a direct hybridization method. In one aspect, the method or kit includes one or more capture oligonucleotides that include one or more nucleic acid sequences that are complementary to a sequence of one or more target nucleic acids in a sample (referred to herein as “target specific capture oligonucleotides”). In one aspect, the method or kit include a plurality of target specific capture oligonucleotides that can be used in a multiplexed array to detect, identify, or quantify a plurality of target analytes in parallel.

In one aspect, the method includes a step of providing a support surface onto which one or more target specific capture molecules are immobilized. In one aspect, the support surface has a flat surface. In one aspect, the support surface is a plate with a plurality of wells, i.e., a “multi-well plate.” Multi-well plates can include any number of wells of any size or shape, arranged in any pattern or configuration. In another aspect, the support surface has a curved surface. In one aspect, the support surface includes an assay module, such as an assay plate, slide, cartridge, bead, or chip. In one aspect, the support surface is provided by one or more particles or “beads”. In one aspect, the support surface includes color coded microspheres. See, for example, Yang et al. (2001) BADGE, BeadsArray for the Detection of Gene Expression, a High-Throughput Diagnostic Bioassay. Genome Res. 11(11): 1888-1898. In one aspect, the support surface includes one or more beads on which one or more target specific capture oligonucleotides are immobilized.

In one aspect, one or more target specific capture molecules are immobilized in binding domains in an array. In one aspect, the support surface includes one or more carbon-based electrodes having one or more surfaces and one or more target specific capture oligonucleotides immobilized in one or more binding domains on one or more surfaces of the one or more carbon-based electrodes.

In one aspect, a sample that contains or is suspected of containing one or more target analytes is contacted with one or more oligonucleotide probes that include one or more sequences complementary to a sequence on one or more target nucleic acids and labeled primers that include sequences that are complementary to one or more target analytes under conditions in which the labeled primer hybridizes to the target analyte. The target analyte can then be amplified using known techniques, such as PCR amplification, to form a labeled reaction product.

In one aspect, a support surface on which one or more target specific capture oligonucleotide sequences are immobilized is contacted with the labeled reaction product under conditions in which one or more labeled reaction products are able to hybridize to their corresponding complementary capture oligonucleotide sequences and identifying, detecting or quantifying the target analyte based on the presence or absence of the label in an array location.

In one aspect, direct hybridization is used to detect, identify or quantify the presence of a virus in a sample. In one aspect, direct hybridization can be used for human papillomavirus (HPV) genotyping. Infection with human papilloma virus (HPV) is the main cause of cervical cancer. More than 200 HPV genotypes have been identified, and approximately 40 are responsible for genital infection. HPV types 16, 18, 26, 31, 33, 35, 39, 45, 51, 52, 53, 56, 58, 59, 66, 68, 73 and 82 are considered carcinogenic. Munoz et al. (2003) Epidemiologic classification of human papillomavirus types associated with cervical cancer. N. Engl. J. Med. 3(48):518.

In one aspect, direct hybridization is used to detect, identify or quantify the presence of bacteria in a sample. In one aspect, direct hybridization is used to detect, identify or quantify Chlamydia trachomatis (C. trachomatis) in a sample. In one aspect, direct hybridization is used to detect, identify or quantify one or more of the three main serotypes for C. trachomatis (serotypes A-C).

In one aspect, direct hybridization is used to detect, identify or quantify the presence of Salmonella enterica in a sample. More than 2600 different serotypes have been identified and can be divided into typhoidal and non-typhoidal servovars. Gal-mor et al. (2014) Same species, different diseases: how and why typhoidal and non-typhoidal Salmonella enterica sevovars differ. Front. Microbiol. 5(391) doi: 10.3389/fmicb.2014.00391.

In one aspect, direct hybridization is used for detection, identification, and/or quantification of a target nucleotide sequence, e.g., therapeutic oligonucleotide, that is in a sample that may contain oligonucleotide metabolites. In one aspect, the sample containing the target nucleotide sequence further includes one or more oligonucleotide metabolites. In one aspect, direct hybridization is used to measure the amount of target nucleotide sequence in a sample relative to oligonucleotide metabolites. In one aspect, direct hybridization is used to determine a pharmacokinetic parameter of a target nucleotide sequence. In one aspect, the pharmacokinetic parameter measured is clearance, volume distribution, plasma concentration, half-life, peak time, peak concentration, rate of availability, or combination thereof. Measurement and interpretation of pharmacokinetic parameters are described herein. In one aspect, the target nucleotide sequence is a therapeutic oligonucleotide. In one aspect, the therapeutic oligonucleotide is an antisense oligonucleotide (ASO). Therapeutic oligonucleotides, ASOs, and their metabolism and pharmacology are described herein.

In one aspect, the oligonucleotide metabolite is shorter than the target nucleotide sequence by 1 or more nucleotides, 2 or more nucleotides, 3 or more nucleotides, 4 or more nucleotides, 5 or more nucleotides, 6 or more nucleotides, 7 or more nucleotides, 8 or more nucleotides, 9 or more nucleotides, 10 or more nucleotides, 15 or more nucleotides, or 20 or more nucleotides. In one aspect, the oligonucleotide metabolite is shorter than the target nucleotide sequence by about 1%, about 2%, about 3%, about 4%, about 5%, about 6%, about 7%, about 8%, about 9%, or about 10%. In one aspect, the target nucleotide sequence is a therapeutic oligonucleotide. In one aspect, the therapeutic oligonucleotide is an antisense oligonucleotide (ASO). In one aspect, the oligonucleotide metabolite is a therapeutic oligonucleotide metabolite.

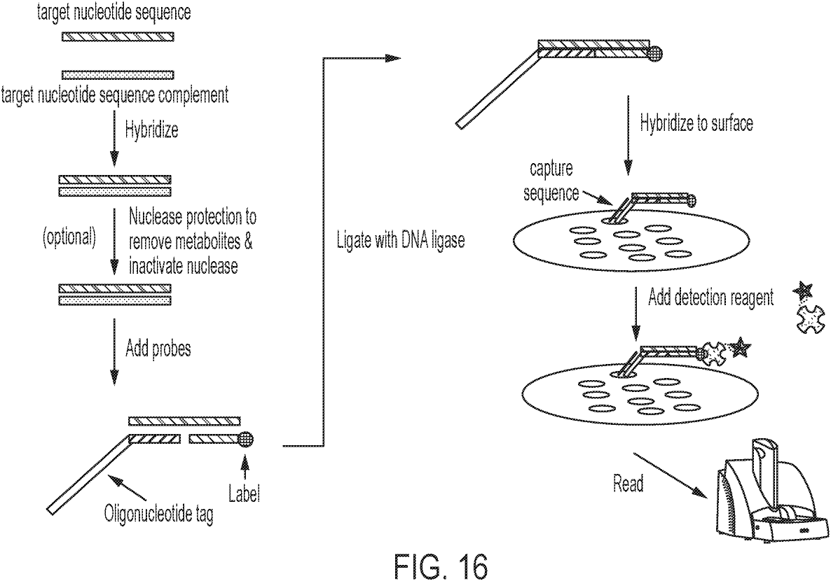

An exemplary embodiment of is illustrated in FIG. 16. In FIG. 16, a sample containing a target nucleotide sequence is contacted with a target nucleotide sequence complement comprising a complementary sequence to the target nucleotide sequence, under conditions wherein the target nucleotide sequence and target nucleotide sequence complement hybridize. In one aspect, the target nucleotide sequence and target nucleotide sequence complement are hybridized over their entire lengths. In one aspect, the target nucleotide sequence analyte and target nucleotide sequence complement hybridize to form a double-stranded complex. In one aspect, the sample containing the target nucleotide sequence further includes one or more oligonucleotide metabolites. Metabolites of target nucleotide sequences, e.g., therapeutic oligonucleotides such as ASOs, are described herein. In one aspect, the method includes removing the oligonucleotide metabolites. In one aspect, a single-strand-specific nuclease is added to the sample while the target nucleotide sequence and target nucleotide sequence complement are hybridized. In one aspect, the single-strand-specific nuclease specifically removes single-stranded oligonucleotide metabolites while being substantially unreactive to the hybridized target nucleotide sequence and target nucleotide sequence complement. In one aspect, the single-strand-specific nuclease additionally removes excess unhybridized target nucleotide sequence complement. Examples of suitable nucleases, including single-strand-specific nucleases, are provided herein. In one aspect, the target nucleotide sequence is a therapeutic oligonucleotide. In one aspect, the therapeutic oligonucleotide is an antisense oligonucleotide. In one aspect, the oligonucleotide metabolite is a therapeutic oligonucleotide metabolite.

In one aspect, after the removal of oligonucleotide metabolite and/or unhybridized target nucleotide sequence complement by the single-strand-specific nuclease, probes capable of hybridizing to adjacent regions of the target nucleotide sequence are added. In one aspect, two adjacent probes, each hybridizing to adjacent regions of the target nucleotide sequence, are ligated to form a reaction product. In one aspect, the probes comprise a targeting probe and a detecting probe as described herein. In one aspect, the targeting probe and detecting probe hybridize over the entire length of the target nucleotide sequence. In one aspect, the targeting probe comprises a oligonucleotide tag. Targeting probes and oligonucleotide tags are further described herein. In one aspect, the oligonucleotide tag is complementary to at least a portion of a capture oligonucleotide immobilized on a support surface. In one aspect, the detecting probe comprises a label. Detecting probes and labels are further described herein. In one aspect, the detecting probe is capable of binding to a detection reagent. In one aspect, the detecting probe comprises a biotin label. In one aspect, the label comprises biotin, and the detection reagent is linked to streptavidin. In another aspect, the label comprises a hapten, and the detection reagent is linked to a hapten binding partner such as an antibody. Labels, detection reagents, and modes of binding between labels and detection reagents are further described herein. In one aspect, the surface is contacted with a detection reagent for binding to the label. In one aspect, the detection reagent is an electrochemiluminescent reagent. In one aspect, the detection reagent comprises an MSD SULFO-TAG. In one aspect, electrochemiluminescence is measured as described herein to detect, identify, and/or quantify the target nucleotide sequence. In one aspect, the amount of target nucleotide sequence in the sample is measured to determine a pharmacokinetic parameter of the target nucleotide sequence. In one aspect, the target nucleotide sequence is a therapeutic oligonucleotide. In one aspect, the therapeutic oligonucleotide is an antisense oligonucleotide. In one aspect, the oligonucleotide metabolite is a therapeutic oligonucleotide metabolite. In one aspect, the target nucleotide sequence includes RNA. In one aspect, the target nucleotide sequence includes miRNA, a therapeutic RNA, an mRNA, an RNA virus or a combination thereof.

### 5. Polymerase Chain Reaction (PCR)

In one aspect, a method or kit is provided for detecting, identifying or quantifying one or more target analytes in a sample using Polymerase Chain Reaction (PCR). In one aspect, a target nucleic acid is amplified using PCR. In one aspect, a method or kit is provided that includes one or more sets of PCR primers, wherein each set of primers includes an upstream primer and a downstream primer. In one aspect, a target nucleotide sequence in a sample is amplified using one or more upstream and downstream PCR primers.

In one aspect, one or more target nucleotide analytes in a sample are amplified using one or more modified upstream or downstream primers. In one aspect, one or more target nucleotide analytes are amplified using one or more upstream primers that include an oligonucleotide tag sequence that is configured to hybridize to a capture oligonucleotide with a complementary sequence. In one aspect, one or more target nucleotide analytes are amplified using one or more downstream primers that include a label. In one aspect, one or more target nucleotide analytes are amplified using one or more downstream primers that include an oligonucleotide tag sequence that is configured to hybridize to a capture oligonucleotide with a complementary sequence. In one aspect, one or more target nucleotide analytes are amplified using one or more upstream primers that include a label.

In one aspect, a target nucleotide sequence is amplified using one or more modified PCR primers to form a PCR reaction product that includes an oligonucleotide tag configured to hybridize to a capture oligonucleotide immobilized on a support surface. In one aspect, a target nucleotide sequence is amplified using one or more modified PCR primers to form a PCR reaction product that includes label. In one aspect, a target nucleotide sequence is amplified using one or more modified PCR primers to form a PCR reaction product that includes an oligonucleotide tag configured to hybridize to a capture oligonucleotide immobilized on a support surface and a label. Methods for labeling PCR reaction products are known and include, for example, labeled deoxynucleotide triphosphates (dNTPs) or modified upstream or downstream primers that include a label.

In one aspect, one or more capture oligonucleotides are immobilized in binding domains in an array on a support surface. In one aspect, the PCR reaction product is captured on the support surface by hybridization of an oligonucleotide tag to its corresponding capture oligonucleotide.

In one aspect, one or more target analytes are detected, identified or quantified using ligation mediated amplification (LM PCR). In one aspect, one or more target analytes are detected, identified or quantified using multiplex “ligation mediated amplification” in combination with the methods described herein. In one aspect, one or more target nucleotide analytes are reverse transcribed using an upstream and a downstream probe. In one aspect, the upstream probe includes a nucleotide sequence that is complementary to a universal primer site, such as T7, an oligonucleotide tag sequence, and a gene specific sequence and the downstream probe includes gene specific fragment contiguous with the gene specific fragment of the upstream probe and a universal primer site, such as T3. In one aspect, the downstream probe is 5′-phosphorylated. In one aspect, the probes are annealed to their targets, free probes are removed and the annealed probes are ligated using a ligase to yield an amplification template. In one aspect, PCR is performed with T3 and 5′-biotinylated T7 primers. In one aspect, capture oligonucleotides that are immobilized to a support surface are contacted with the biotinylated amplicons under conditions in which the oligonucleotide tags hybridize to their corresponding capture oligonucleotides. In one aspect, the captured labeled amplicons are incubated with labeled streptavidin, for example, SULFO-TAG labeled streptavidin so that the captured labeled amplicons can be detected, identified or quantified. See, for example, Peck et al. (2006) A method for high-throughput gene expression signature analysis. Genome Biol. 7(7):R61.

In one aspect, the target analyte is cDNA. In one aspect, the target analyte is mRNA. In one aspect, cDNA is synthesized from poly-A tailed mRNAs using oligo-dT primers. In one aspect, cDNA can be generated from mRNA using random primed cDNA synthesis.

### 6. Nuclease Protection Assay (NPA)

In one aspect, a method or kit is provided for detecting, identifying or quantifying one or more target analytes in a sample using a nuclease protection assay. In one aspect, a nuclease protection assay is used to detect, identify or quantify a target analyte in a sample that contains or is suspected of containing the target analyte. In one aspect, the target analyte includes a single stranded nucleic acid, including, for example, single stranded RNA. In one aspect, the target analyte includes microRNA (miRNA). In one aspect, the sample is contacted with one or more single-stranded probes that include a sequence that is complementary to a sequence of the target analyte and an oligonucleotide tag sequence under conditions in which the target analyte hybridizes to the probe to form a tagged reaction product. In one aspect, the probe is a DNA/RNA hybrid probe that includes a single stranded DNA tag sequence and a single stranded RNA sequence that is complementary to a nucleic acid sequence of the target analyte. In one aspect, the hybrid probe includes a biotin label.

In one aspect, a support surface onto which one or more capture oligonucleotides are immobilized is contacted with a mixture that includes the tagged reaction product under conditions in which one or more oligonucleotide tag sequences hybridize to their corresponding capture oligonucleotide sequences immobilized on the support surface. After the oligonucleotide tags are allowed to hybridize to their corresponding capture oligonucleotides on the support surface, the support surface is washed and contacted with an RNase specific for single stranded RNA, for example, RNase A or RNase I under conditions in which the RNase can digest single-stranded RNA molecules and remove excess probe bound to spots with no hybridized target RNA and cleave any mismatched sites between the probe and target RNA.

In one aspect, the miRNA analysis includes a step-down probe hybridization step, in which the DNA/RNA chimeric probes hybridize to target miRNAs during incremental reductions in annealing temperature.

In one aspect, a nuclease protection assay (NPA) with direct surface coating is used for detection, identification, and/or quantification of a target nucleotide sequence that is in a sample that may contain degradation products of the target nucleotide sequence, also referred to as oligonucleotide metabolites. In one aspect, the sample containing the target nucleotide sequence further includes one or more oligonucleotide metabolites. In one aspect, an NPA with direct surface coating is used to measure the amount of target nucleotide sequence in a sample relative to oligonucleotide metabolites. In one aspect, an NPA with direct surface coating is used to determine a pharmacokinetic parameter of a therapeutic oligonucleotide. In one aspect, the pharmacokinetic parameter measured is clearance, volume distribution, plasma concentration, half-life, peak time, peak concentration, rate of availability, or combination thereof. Measurement and interpretation of pharmacokinetic parameters are described herein. In one aspect, the target nucleotide sequence is a therapeutic oligonucleotide. In one aspect, the therapeutic oligonucleotide is an antisense oligonucleotide (ASO). In one aspect, the oligonucleotide metabolite is a therapeutic oligonucleotide metabolite. Therapeutic oligonucleotides, antisense oligonucleotides, and their metabolism and pharmacology are described herein.

In one aspect, the oligonucleotide metabolite is shorter than the target nucleotide sequence by 1 or more nucleotides, 2 or more nucleotides, 3 or more nucleotides, 4 or more nucleotides, 5 or more nucleotides, 6 or more nucleotides, 7 or more nucleotides, 8 or more nucleotides, 9 or more nucleotides, 10 or more nucleotides, 15 or more nucleotides, or 20 or more nucleotides. In one aspect, the oligonucleotide metabolite is shorter than the target nucleotide sequence by about 1%, about 2%, about 3%, about 4%, about 5%, about 6%, about 7%, about 8%, about 9%, or about 10%. In one aspect, the target nucleotide sequence is a therapeutic oligonucleotide. In one aspect, the therapeutic oligonucleotide is an antisense oligonucleotide (ASO). In one aspect, the oligonucleotide metabolite is a therapeutic oligonucleotide metabolite. In one aspect, the therapeutic oligonucleotide is detected without amplifying the therapeutic oligonucleotide. In one aspect, the therapeutic oligonucleotide in the sample is detected without a nucleic acid extraction step.

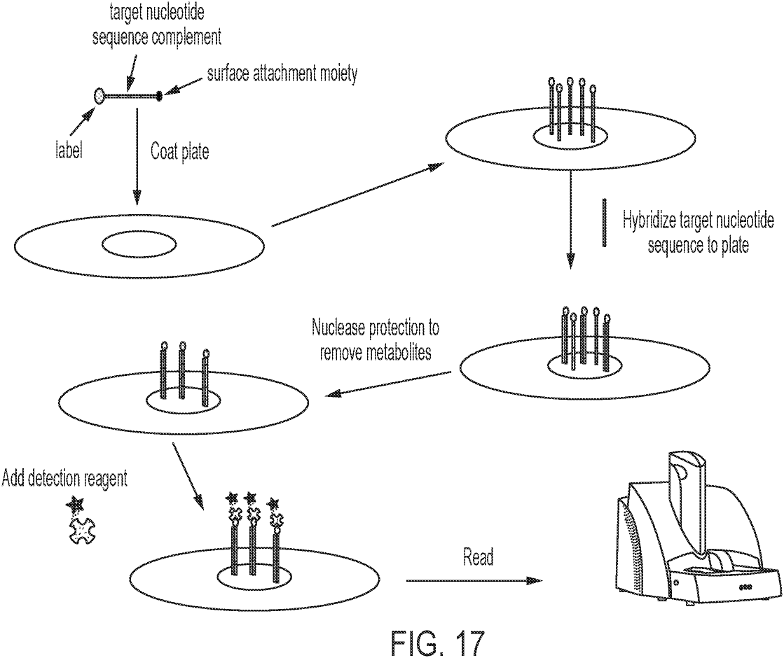

An exemplary embodiment is illustrated in FIG. 17. In FIG. 17, a target nucleotide sequence complement comprising a complementary sequence to a target nucleotide sequence is used as the capture oligonucleotide. The target nucleotide sequence complement comprises a label at one end and a surface attachment moiety on the other end. Methods of immobilizing a capture oligonucleotide to a surface are described herein and include, e.g., electrostatic interactions, complementary binding partners, complementary reactive functional groups, linkers (e.g., cross-linking agents including reactive functional groups), and the like. In one aspect, the surface is coated with the target nucleotide sequence complement via the surface attachment moiety of the target nucleotide sequence complement. In one aspect, the surface attachment moiety comprises a thiol. In one aspect, the surface attachment moiety comprises biotin.

In one aspect, the target nucleotide sequence complement-coated surface is contacted with a sample containing the target nucleotide sequence, under conditions wherein the target nucleotide sequence complement and the target nucleotide sequence hybridize. In one aspect, the target nucleotide sequence and the target nucleotide sequence complement are hybridized over their entire lengths. In one aspect, the target nucleotide sequence and target nucleotide sequence complement hybridize to form a double-stranded complex. In one aspect, the sample containing the target nucleotide sequence further includes one or more oligonucleotide metabolites. Metabolites of target nucleotide sequences, e.g., therapeutic oligonucleotides such as ASOs, are described herein. In one aspect, the method includes removing the oligonucleotide metabolites. In one aspect, a single-strand-specific nuclease is added to the sample while the target nucleotide sequence and target nucleotide sequence complement are hybridized. In one aspect, the single-strand-specific nuclease specifically removes single-stranded oligonucleotide metabolites while being substantially unreactive to the hybridized target nucleotide sequence-target nucleotide sequence complement. Examples of suitable nucleases, including single-strand-specific nucleases are provided herein.

In one aspect, after removal of the oligonucleotide metabolite by the single-strand-specific nuclease, the surface is contacted with a detection reagent capable of binding to the label on the target nucleotide sequence complement. In one aspect, the label comprises biotin, and the detection reagent is linked to streptavidin. In another aspect, the label comprises a hapten, and the detection reagent is linked to a hapten binding partner such as an antibody. Labels, detection reagents, and modes of binding between labels and detection reagents are further described herein. In one aspect, the detection reagent is an electrochemiluminescent reagent. In one aspect, the detection reagent comprises an MSD SULFO-TAG. In one aspect, electrochemiluminescence is measured as described herein to detect, identify, and/or quantify the target nucleotide sequence. In one aspect, the amount of target nucleotide sequence in the sample is measured to determine a pharmacokinetic parameter of the target nucleotide sequence. In one aspect, the target nucleotide sequence is a therapeutic oligonucleotide. In one aspect, the therapeutic oligonucleotide is an antisense oligonucleotide. In one aspect, the oligonucleotide metabolite is a therapeutic oligonucleotide metabolite. In one aspect, the target nucleotide sequence includes RNA. In one aspect, the target nucleotide sequence includes miRNA, a therapeutic RNA, an mRNA, an RNA virus or a combination thereof.

In a further aspect, the target nucleotide sequence is a small nucleic acid, e.g., at least about 15 base pairs, at least about 16 base pairs, at least about 17 base pairs, at least about 18 base pairs, at least about 19 base pairs, or at least about 20 base pairs and up to about 20 base pairs in length, up to about 25 base pairs in length, up to about 30 base pairs in length, up to about 40 base pairs in length or up to about 50 base pairs in length. In one aspect, the probe for detecting such small nucleic acid targets includes at least about 8 base pairs, at least about 9 base pairs, at least about 10 base pairs, at least about 11 base pairs, or at least about 12 base pairs and up to about 20 base pairs in length, up to about 25 base pairs in length, up to about 30 base pairs in length, up to about 40 base pairs in length or up to about 50 base pairs in length, and the probe and the small nucleic acid target are ligated after hybridizing another as described herein.

### 7. Hybridization/Protection Assay

In one aspect, a hybridization/protection assay is used for detection, identification, and/or quantification of a target nucleotide sequence, e.g., a therapeutic oligonucleotide, that is in a sample that may contain oligonucleotide metabolites. In one aspect, the sample containing the target nucleotide sequence further includes one or more oligonucleotide metabolites. In one aspect, the hybridization/protection assay is used to measure the amount of target nucleotide sequence in a sample relative to oligonucleotide metabolites. In one aspect, the hybridization/protection assay is used to determine a pharmacokinetic parameter of a therapeutic oligonucleotide. In one aspect, the pharmacokinetic parameter measured is clearance, volume distribution, plasma concentration, half-life, peak time, peak concentration, rate of availability, or combination thereof. Measurement and interpretation of pharmacokinetic parameters are described herein. In one aspect, the target nucleotide sequence is a therapeutic oligonucleotide. In one aspect, the therapeutic oligonucleotide is an antisense oligonucleotide (ASO). Therapeutic oligonucleotides, ASOs, and their metabolism and pharmacology are described herein.

In one aspect, the oligonucleotide metabolite is shorter than the target nucleotide sequence by 1 or more nucleotides, 2 or more nucleotides, 3 or more nucleotides, 4 or more nucleotides, 5 or more nucleotides, 6 or more nucleotides, 7 or more nucleotides, 8 or more nucleotides, 9 or more nucleotides, 10 or more nucleotides, 15 or more nucleotides, or 20 or more nucleotides. In one aspect, the oligonucleotide metabolite is shorter than the target nucleotide sequence by about 1%, about 2%, about 3%, about 4%, about 5%, about 6%, about 7%, about 8%, about 9%, or about 10%. In one aspect, the target nucleotide sequence is a therapeutic oligonucleotide. In one aspect, the therapeutic oligonucleotide is an antisense oligonucleotide (ASO). In one aspect, the oligonucleotide metabolite is a therapeutic oligonucleotide metabolite. In one aspect, the therapeutic oligonucleotide is detected without amplifying the therapeutic oligonucleotide. In one aspect, the therapeutic oligonucleotide in the sample is detected without a nucleic acid extraction step.

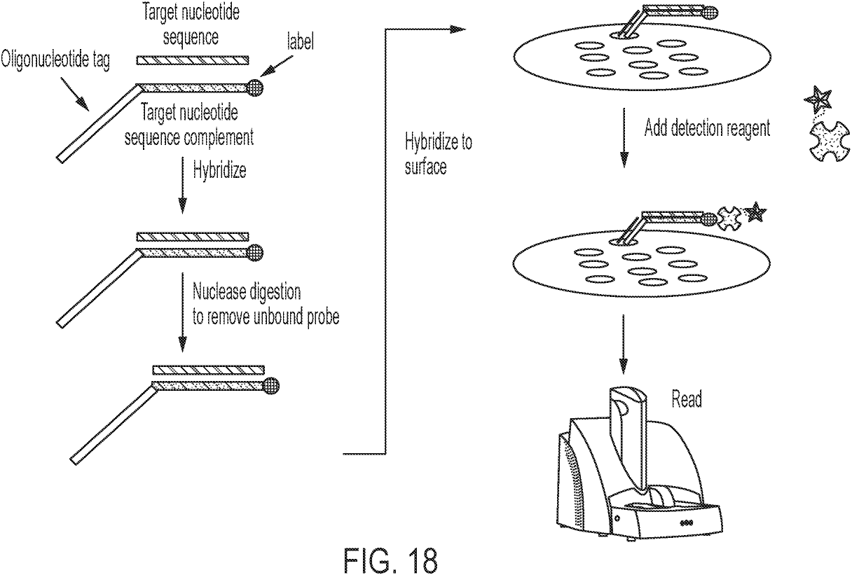

An exemplary embodiment is illustrated in FIG. 18. In FIG. 18, a sample containing a target nucleotide sequence is contacted with a target nucleotide sequence complement probe comprising (i) a target nucleotide sequence complement sequence complementary to the target nucleotide sequence; (ii) an oligonucleotide tag and (iii) a label. In one aspect, the oligonucleotide tag of the target nucleotide sequence complement probe is complementary to at least a portion of a capture oligonucleotide immobilized on a support surface. In one aspect, the oligonucleotide tag of the target nucleotide sequence is double-stranded, and one strand of the oligonucleotide tag is complementary to at least a portion of a capture oligonucleotide immobilized on a support surface. Oligonucleotide tags and capture oligonucleotides are further described herein. In one aspect, the label of the target nucleotide sequence complement probe is capable of binding to a detection reagent. In one aspect, the label comprises biotin, and the detection reagent is linked to streptavidin. In another aspect, the label comprises a hapten, and the detection reagent is linked to a hapten binding partner such as an antibody. Labels, detection reagents, and modes of binding between labels and detection reagents are further described herein.

In one aspect, the target nucleotide sequence hybridizes to the target nucleotide sequence complement probe. In one aspect, the target nucleotide sequence and the target nucleotide sequence complement probe hybridize over the entire length of the target nucleotide sequence and the target nucleotide sequence complement sequence. In one aspect, the oligonucleotide tag is double-stranded, and the target nucleotide sequence and target nucleotide sequence complement sequence hybridize to form a double-stranded complex. In one aspect, the sample containing the target nucleotide sequence further includes one or more oligonucleotide metabolites. Metabolites of target nucleotide sequences, e.g., therapeutic oligonucleotides such as ASOs, are described herein. In one aspect, the method includes removing the oligonucleotide metabolites. In one aspect, a single-strand-specific nuclease is added to the sample while the target nucleotide sequence and target nucleotide sequence complement are hybridized. In one aspect, the single-strand-specific nuclease specifically removes single-stranded oligonucleotide metabolites while being substantially unreactive to the hybridized target nucleotide sequence-target nucleotide sequence complement. In one aspect, the single-strand-specific nuclease additionally removes excess unhybridized target nucleotide sequence complement probe. Examples of suitable nucleases, including single-strand-specific nucleases are provided herein.

In one aspect, after the removal of oligonucleotide metabolite and/or unhybridized target nucleotide sequence complement probe, the hybridized target nucleotide sequence-target nucleotide sequence complement probe is immobilized onto the support surface via binding of the oligonucleotide tag on the target nucleotide sequence complement probe to the capture oligonucleotide on the surface. In one aspect, the oligonucleotide metabolite and/or unhybridized target nucleotide sequence complement probe is removed prior to immobilization of the hybridized target nucleotide sequence-target nucleotide sequence complement probe to provide improved sensitivity compared with simultaneous removal/immobilization, or immobilization followed by removal formats. In one aspect, a detection reagent is added to the surface, and the detection reagent binds to the label on the target nucleotide sequence complement probe. In one aspect, the detection reagent is an electrochemiluminescent reagent. In one aspect, the detection reagent comprises an MSD SULFO-TAG. In one aspect, electrochemiluminescence is measured as described herein to detect, identify, and/or quantify the target nucleotide sequence. In one aspect, the amount of target nucleotide sequence in the sample is measured to determine a pharmacokinetic parameter of the target nucleotide sequence. In one aspect, the target nucleotide sequence is a therapeutic oligonucleotide. In one aspect, the therapeutic oligonucleotide is an antisense oligonucleotide. In one aspect, the oligonucleotide metabolite is a therapeutic oligonucleotide metabolite. In one aspect, the target nucleotide sequence includes RNA. In one aspect, the target nucleotide sequence includes miRNA, a therapeutic RNA, an mRNA, an RNA virus or a combination thereof.

### K. Sample

The method or kit described herein are suitable for detecting one or more target analytes in a sample that contains or is suspected of containing the one or more target analytes. In one aspect, the target analyte includes a target nucleotide sequence. In another aspect, the target analyte includes a target protein. In one aspect, the sample includes or is suspected to include one or more prokaryotic or eukaryotic DNA or RNA sequences of interest. In one aspect, the sample is a biological sample obtained from an organism such as a human or other mammal, including, but not limited, to non-human primates, dogs, cats, cattle, sheep, poultry, horses; or other organisms such as plants, bacteria, fungi, protists or viruses. In one aspect, the biological sample includes a solid material such as a tissue, cells, a cell extract, or a biopsy; or a biological fluid such as urine, blood, saliva, amniotic fluid, exudate from a region of infection or inflammation, a mouth wash containing buccal cells, cerebral spinal fluid, or synovial fluid. In one aspect, the sample is isolated from an individual. In another aspect, the sample is derived from a group of individuals. In one aspect, the sample includes one or more, or multiple individual samples or pooled samples.

In one aspect, the sample includes one or more target DNA sequences, including, but not limited to, single or double stranded DNA, including, but not limited to genomic DNA, mitochondrial DNA, cDNA, whole genome amplified DNA, or combinations thereof. In another aspect, the sample includes one or more target RNA sequences, including, but not limited to, single or double stranded RNA, including, but not limited to, ribosomal RNA, mRNA, miRNA, siRNA, RNAi, or combinations thereof. In another aspect, the sample includes or is suspected to include one or more target nucleotide sequences that are amplicons such as PCR products, plasmids, cosmids, DNA libraries, yeast artificial chromosome (YAC), bacterial artificial chromosome (BAC), synthetic oligonucleotides, restriction fragments, DNA/RNA hybrids, PNA (peptide nucleic acid) or a DNA/RNA mosaic nucleic acid. For a double-stranded nucleic acid, the target nucleotide sequence can be present in either strand. In one aspect, the sample does not include ethylenediaminetetraacetic acid (EDTA).

In one aspect, the sample includes one or more target nucleotide sequences, e.g., therapeutic oligonucleotides, wherein the sample also may contain oligonucleotide metabolites. A “therapeutic oligonucleotide” as used herein refers to an oligonucleotide capable of interacting with a biomolecule to provide a therapeutic effect. In one aspect, the therapeutic oligonucleotide is an antisense oligonucleotide (ASO). ASOs are single stranded oligonucleotides that are typically from about 5, 10, 15, 20 or 25 nucleotides to about 30, 35, 40, 45 or 50 nucleotides in length. ASOs are capable of influencing RNA processing and/or modulating protein expression. An ASO is a single-stranded oligonucleotide that binds to single-stranded RNA to inactivate the RNA. In one aspect, the ASO binds to messenger RNA (mRNA) for a gene, thereby inactivating the gene. In one aspect, the gene is a disease gene. Thus, the ASO can inactivate mRNA of a disease gene to prevent or ameliorate production of a particular disease-causing protein. In one aspect, the ASO comprises DNA, RNA, or combination thereof.

Oligonucleotides, e.g., therapeutic oligonucleotides such as ASOs, in a sample can degrade, e.g., shorten, over time, due to various factors such as presence of nucleases, temperature, pH, salt concentration, and the like. In certain aspects, degradation of therapeutic oligonucleotide in a sample is indicative of a pharmacodynamic response to the therapeutic oligonucleotide. Degraded or shortened therapeutic oligonucleotides, also referred to herein as therapeutic oligonucleotide metabolites, may lose therapeutic effectiveness. In one aspect, the sample includes a therapeutic oligonucleotide and one or more therapeutic oligonucleotide metabolites. In one aspect, the therapeutic oligonucleotide metabolite is shorter than the therapeutic oligonucleotide by 1 or more nucleotides, 2 or more nucleotides, 3 or more nucleotides, 4 or more nucleotides, 5 or more nucleotides, 6 or more nucleotides, 7 or more nucleotides, 8 or more nucleotides, 9 or more nucleotides, 10 or more nucleotides, 15 or more nucleotides, or 20 or more nucleotides. In one aspect, the therapeutic oligonucleotide metabolite is shorter than the therapeutic oligonucleotide by about 1%, about 2%, about 3%, about 4%, about 5%, about 6%, about 7%, about 8%, about 9%, or about 10%.

In one aspect, the methods provided herein are used to measure the amount of therapeutic oligonucleotide in a sample relative to therapeutic oligonucleotide metabolites. In one aspect, the pharmacokinetic parameters of a therapeutic oligonucleotide is determined by measuring the rate and/or amount of degradation of the therapeutic oligonucleotide in a biological environment, e.g., a patient. Thus, in one aspect, the methods provided herein are used to determine a pharmacokinetic parameter of a therapeutic oligonucleotide. In one aspect, the pharmacokinetic parameter measured is clearance, volume distribution, plasma concentration, half-life, peak time, peak concentration, rate of availability, or combination thereof. Measurement and interpretation of pharmacokinetic parameters are further described herein.

In one aspect, the sample includes one or more anti-drug antibodies (ADA). In one aspect, the ADA binds a therapeutic polypeptide, including, but not limited to, a therapeutic protein or a therapeutic antibody. In one aspect, the ADA binds a therapeutic oligonucleotide, including, but not limited to, antisense oligonucleotides (ASOs), short interfering RNAs, microRNAs, and synthetic guide strands for CRISPR/Cas. In one aspect, the ADA is capable of binding to the biopharmaceutical product. In one aspect, the ADA is capable of inhibiting functional activity of the therapeutic product.

In one aspect, the sample includes one or more unamplified target nucleotide sequences. In another aspect, the sample includes one or more target nucleotides sequence obtained by amplification or cloning of the sequences from a biological sample. Amplification can be achieved by methods including, but not limited to, polymerase chain reaction (PCR), whole genome amplification (WGA), reverse transcription followed by the polymerase chain reaction (RT-PCR), strand displacement amplification (SDA), or rolling circle amplification (RCA).

In one aspect, the sample includes or is suspected of including one or more target proteins. In one aspect, the target protein includes a DNA binding protein, including, for example, a protein with a DNA binding domain that can bind to single- or double-stranded DNA. Examples of DNA binding proteins include, but are not limited to, transcription factors, polymerases, nucleases and histones. In one aspect, the DNA binding protein binds to a specific DNA sequence, for example, a transcription factor.

In one aspect, one or more target analytes are purified from a biological sample. Methods for purifying target analytes from a sample are known. Methods for purifying nucleotide sequences from a biological sample are known and include, for example, high performance liquid chromatography (HPLC), for example, reverse phase high performance liquid chromatography (RP-HPLC) or anion exchange high pressure liquid chromatography (AEX HPLC) or polyacrylamide gel electrophoresis (PAGE). Methods for purifying a protein from a biological sample are known and include, for example, chromatography, such as size exclusion chromatography, high performance liquid chromatography (HPLC), hydrophobic interaction chromatography (HIC), ion exchange chromatography, affinity chromatography and electrophoresis.

In one aspect, the sample includes at least about 1 µg, 2 µg, 3 µg, 4 µg, 5 µg, 6 µg, 7 µg, 8 µg, 9 µg or 10 µg and up to about 20 µg, 25 µg, 30 µg, 35 µg, 40 µg, 45 µg or 50 µg, or between about 1 µg and about 50 µg, or between about 5 µg and about 20 µg of one or more target analytes, for example, genomic DNA purified from a cell line or whole genome amplified DNA. In one aspect, the sample includes at least about 0.1 µL. 0.5 µL, 1 µL, 2 µL, 3 µL, 4 µL, 5 µL and up to about 6 µL, 7 µL, 8 µL, 9 µL, 10 µL, 15 µL, 20 µL or 25 µL or between about 1 µL to about 25 µL, or between about 0.1 µL to about 5 µL of a sample containing one or more target analytes, for example, a sample containing one or more amplification products, for example, PCR amplicons generated from cell line DNA. In one aspect, the sample has an analyte concentration of at least about 1 ng/µL, 5 ng/µL, or 10 ng/µL and up to about 25 ng/µL, 50 ng/µL or 100 ng/µL.

In one aspect, the sample includes at least one copy of the target analyte. In one aspect, the sample includes the target nucleic acid in copy numbers less than 10⁷, 10⁶ 10⁵, 10⁴, 10³, 10², or 10¹. In one aspect, these copies are present in between about 0.001 mL and about 1 mL of sample, or in less than about 1 mL, 0.1 mL, 0.01 mL, or 0.001 mL of sample.

### L. Sample Amplification

Although the method or kits described herein can be used in connection with samples in which one or more target nucleotide sequences have not been amplified, it may be desirable to include an amplification step to increase the quantity of target nucleotide in the sample. For example, it may be desirable to amplify the target nucleotide sequence when the target nucleotide sequence includes one or more rare mutations, for example, one or more rare or low allele fraction mutations associated with cancer.

In one aspect, the target nucleotide sequence is amplified by polymerase chain reaction (PCR). Methods for PCR amplification are known. See, for example by Saiki et al. Primer-Directed Enzymatic Amplification of DNA with a Thermostable DNA Polymerase, Science, 239:487-491. Briefly, in PCR amplification, a target nucleotides sequence is contacted with two oligonucleotide primers that flank a specific nucleotide sequence to be amplified. Repeated cycles of heat denaturation, annealing of the primers to their complementary sequences and extension of the annealed primers with DNA polymerase result in the exponential accumulation of the target fragment approximately 2ⁿ, where n is the number of cycles.

In another aspect, the target nucleotide sequence is amplified using rolling circle amplification (RCA), an isothermal nucleic acid (e.g., DNA or RNA) amplification technique in which a polymerase continuously adds single nucleotides to a primer annealed to a circular template, resulting in a long single stranded DNA or RNA sequence containing a plurality, for example, tens to hundreds, of tandem repeats that are complementary to the circular template.

In another aspect, whole genome amplification (WGA) is used to amplify a genomic DNA sample. Methods for whole genome amplification are known and include, for example, Multiple Displacement Amplification (MDA), Degenerate Oligonucleotide PCR (DOP-PCR) and Primer Extension Preamplification (PEP). While DOP-PCR and PEP are based on standard PCR techniques, MDA uses an isothermal reaction setup.

In some aspects, amplification includes the use of one or more oligonucleotide primers which are used by polymerases to initiate DNA or RNA synthesis. Primers can be deoxyribonucleic acid (DNA), ribonucleic acid (RNA), peptide nucleic acid (PNA), locked nucleic acid (LNA), phosphorothioate-linkage containing DNA or a combination thereof and include nucleotide analogs or modified nucleotides. Generally, primers are single stranded oligonucleotides between about 10 and about 100, or about 15 and about 30, or at least about 10, 15 or 20 and up to about 25, 30, 35, 40, 45 or 50 nucleotides in length. In some aspects, the oligonucleotide primers are specific primers, which are complementary to certain regions of the target nucleotide sequence such that the region of the template that is amplified is defined by the primers. Methods for preparing oligonucleotide primers are known. In one aspect, commercially available amplification primers can be used. In one aspect, the primers are at least about 80%, 85%, 90%, 95%, 96%, 97%, 98%, 99% or 100% pure.

In one aspect, the sample includes a PCR product. In one aspect, the PCR product is between about 25 bp and about 500 bp, or about 50 bp and about 300 bp or about 75 bp and about 200 bp in length. In one aspect, the PCR primers have a melting temperature that is similar (i.e., within about 5° C. or 1° C.) of other primers used in a multiplexed PCR assay.

### M. Detection

In one aspect, a target analyte is detected, identified or quantified in an array. In one aspect, a reaction product, including, for example, a PCR reaction product, an OLA reaction product, a PEA reaction product, a sandwich complex or an NPA reaction product as described herein can be detected, identified or quantified in an array. In one aspect, the array is a multiplex array, and the target analyte is detected, identified or quantified by detection of a label attached to an immobilized target molecule on the array. In one aspect, a support surface is contacted with a hybridization mixture containing a tagged reaction product and the reaction product is immobilized onto the support surface by hybridization of the single stranded oligonucleotide tag with its corresponding complementary capture oligonucleotide. In one aspect, the reaction product is amplified before the solid support is contacted with the reaction product. In one aspect, the reaction product is amplified at least about 10×, 20×, 30×, 40× or 50×. In one aspect, the hybridization mixture includes a hybridization buffer. In one aspect, the presence or amount of reaction product can be detected, identified or quantified based on the label attached to the reaction product. In one aspect, the support surface is washed with a wash buffer after the reaction product is immobilized thereon.

In one aspect, the presence of one or more target nucleotides sequences is detected, identified or quantified based on the detection of the reaction product immobilized on the support surface. In one aspect, the presence of the immobilized reaction product is detected by monitoring light emission from a label on the reaction product, including, but not limited to, fluorescence, time-resolved fluorescence, fluorescence resonance energy transfer (FRET), fluorescence polarization (FP), luminescence, chemiluminescence, bioluminescence, phosphorescence, light scattering or electrode induced luminescence. In another aspect, the label includes enzymes or other chemically reactive species with a chemical activity that leads to a measurable signal such as light scattering, absorbance, fluorescence, etc. Examples of enzyme labels include, but are not limited to, horseradish peroxidase or alkaline phosphatase. In one aspect, the label is a detectable hapten, including, but not limited to, biotin, fluorescein or digoxigenin. In one aspect, the reaction product includes a biotin label.

In one aspect, the reaction product is immobilized on one or more binding domains located on the support surface. In one aspect, one or more binding domains are located on one or more electrodes and detecting, identifying or quantifying includes applying a voltage waveform to one or more electrodes to stimulate the labels on the captured reaction products to produce an electrochemical or luminescent signal. In one aspect, detecting, identifying or quantifying includes measuring an electrochemiluminescent signal and correlating the signal with the presence or an amount of target nucleotide sequence in a sample. In one aspect, the intensity of the emitted light is proportional to the amount target in the sample such that the emitted light can provide a quantitative determination of the amount of target nucleotide in the sample.

In one aspect, the support surface is contacted with a detection mixture after the reaction product is immobilized thereon. In one aspect, the detection mixture includes an electrochemiluminescent label. Examples of electrochemiluminescent labels include: i) organometallic compounds where the metal is from, for example, the noble metals of group VIII, including Ru-containing and Os-containing organometallic compounds such as the tris-bipyridyl-ruthenium (RuBpy) moiety and ii) luminol and related compounds. In one aspect, the detection mixture also includes one or more electrochemiluminescence co-reactants, and one or more additional components such as a pH buffering agent, detergent, preservative, anti-foaming agent, salt, metal ion or metal chelating agent. The term “electrochemiluminescent co-reactant” refers to species that participate with the electrochemiluminescent label to and include, but are not limited to, tertiary amines, such as tripropylamine (TPA), oxalate ion, ascorbic acid and persulfate for RuBpy and hydrogen peroxide for luminol. Methods for measuring electrochemiluminescence are known and instruments for making the measurements are commercially available. For example, multiplexed measurement of analytes using electrochemiluminescence is used in the Meso Scale Diagnostics, LLC, MULTI-ARRAY® and SECTOR® Imager line or products (see, e.g., U.S. Pat. Nos. 7,842,246 and 6,977,722, the disclosures of which are incorporated herein by reference in their entireties).

In one aspect, biotin is covalently attached to the reaction product and the detection mixture includes a streptavidin-conjugated label which binds to the immobilized reaction product through the avidin moiety. In one aspect, the streptavidin-conjugated label is an electrochemiluminescent (ECL) label. In one aspect, the electrochemiluminescent label is an n-hydroxysuccinimide ester, such as the Sulfo-TAG NHS-Ester (Meso Scale Diagnostics).

In one aspect, the kit or method are used to detect, identify or quantify one or more single nucleotide polymorphisms (SNP) in one or more target nucleotide sequences. In one aspect, the presence of a SNP of interest is detected by determining the ratio between wild-type and variant allele in a sample. In one aspect, the ratio is determined by determining the ratio of detectable label for the wild-type and variant allele present in an sample. In one aspect, the ratio of electrochemiluminescent label for the wild-type and variant allele is determined. The following formulae can be used to determine the ratio of wild-type or variant allele present in a sample:
ECL Ratio WT = (SignalWT-) / (SignalWT-Bkg + SignalMUT-Bkg)
ECL Ratio MUT = (SignalMUT-Bkg) / (SignalWT-Bkg + SignalMUT-Bkg)
wherein SignalWT is the ECL signal detected for the wild-type allele, SignalMUT is the signal detected for the variant allele and Bkg is the background signal. In one aspect, the background signal is specific for the binding domain corresponding to the wild-type or variant allele, as background signals can vary between binding domains. In one aspect, the background value is the mean value for replicate spots in two wells for a “no ligase control” sample.

The ratio estimates the percent of wild type and variant sequence present in a sample. In one aspect, the possibilities for the sample are: homozygous wild-type, heterozygous, or homozygous variant. In one aspect, the ratio for homozygous wild-type or variant should be greater than about 0.8, heterozygous should be between about 0.3 and about 0.7, and absence of the variant (or wild-type) should be less than about 0.2. The ratio for a homozygous allele can be greater than 1.0 due to signal variability. Similarly, the absence of an allele can result in a ratio that is less than zero, due to background subtraction.

In one aspect, the kit or method is used to detect one or more rare or low-allele fractions of cancer mutations. In one aspect, the frequency of a rare or low-allele fraction mutation present in a sample is determined by generating a calibration curve from the ECL signals using the following formula:
ECL Ratio MUT = (SignalMUT) / (SignalWT + SignalMUT).

Background subtraction is not necessary in preparing the calibration curve, as all signals are compared against the calibration curve, and background is accounted for in the fit. The calibration curve establishes the lowest percent variant allele detectable for a given allele and fitting sample data back to the curve allows for the determination of the percent variant present in each sample.

In one aspect, the assay has a limit of detection (LOD) of between about 1×10⁵ and 10×10⁵, or less than about 10×10⁵, 9×10⁵, 8×10⁵, 7×10⁵, 6×10⁵, 5×10⁵, 4×10⁵, 3×10⁵, 2×10⁵, or 1×10⁵ molecules per well. In one aspect, the LOD for an OLA-based assay is between about 1×10⁵ and 5×10⁵, or about 2×10⁵ molecules per well. In one aspect, the LOD for a PEA-based assay is between about 4×10⁵ and about 6×10⁵, or about 5×10⁵, molecules per well.

### N. Method of Use

Described herein are methods and kits for identifying, detecting or quantifying one or more target analytes in a sample. In one aspect, the target analyte is nucleotide sequence. In another aspect, the target analyte is a protein. In one aspect, the target analyte contains or is suspected of containing a wild-type nucleotide or peptide sequence. In one aspect, the target nucleotide sequence contains or is suspected of containing a mutation, such as a deletion, addition, substitution, transition, transversion, rearrangement, or translocation. In one aspect, the mutation includes a missense, nonsense, silent, or splice-site mutation.

In one aspect, the method or kit is used to detect, identify, or quantify one or more nucleotide sequences in a sample. In one aspect, the method or kit is used to detect, identify or quantify one or more single nucleotide polymorphisms (SNP), copy number variants (CNV), or other sequence variants or mutations in a sample.

In one aspect, the method or kit is used to identify, detect or quantify one or more target nucleotide sequences in a sample containing mixtures of nucleic acids, for example, from multiple genomes or species, multiple individuals, or biological samples such as tumor samples that are derived from mixtures of tissues or cells. In one aspect, the method or kit is used to detect one or more nucleotide sequences that may be present in the sample. In one aspect, the method or kit is used to detect a single nucleotide variant that is present at a frequency of at least about 50% or up to about 100%. In one aspect, the variant is absent. In another aspect, the method or kit is used to detect one or more single nucleotide polymorphisms that are present in more than about 1% of the nucleotide sequences present in the sample. In one aspect, the method or kit is used to detect single nucleotide polymorphisms that are present in less than about 5% or 10% of the nucleotide sequences present in the sample. In one aspect, the method or kit can be used to identify, detect or quantify a nucleic acid mutation in a biological sample that contains a heterogeneous mixture of nucleotide sequences with a mutation in the target region as well as wild-type nucleic acid sequences, in which the mutation may be present in between about 1% and about 5% of the target nucleotide sequences. In one aspect, the method or kit is used to analyze one or more mutations in a target nucleotide that is indicative of the presence of cancerous or precancerous tissue in a biological sample or a tissue biopsy, including for example, single-nucleotide cancer-associated mutations indicative of cancer, such as prostate, breast, colon, pancreatic or cervical cancer. In one aspect, the method or kit is used to detect mutations present in less than about 0.01%, 0.02%, 0.03%, 0.04% or 0.05% of the nucleotide sequences in the sample. In one aspect, the method or kit is used to detect one or more target nucleotide sequences present in a blood sample, extracellular fluids, extracellular vesicles or a liquid biopsy. In one aspect, the method or kit is used to detect one or more mutations of interest in oncology, including, but not limited to mutations in circulating tumor cells in a background of normal cells, or detection of tumor-derived cell-free DNA in blood. In one aspect, the method or kit is used for identifying, detecting or quantifying one or more mutations important for drug development.

In one aspect, the method or kit is used to detect, identify or quantify RNA in a sample. In one aspect, the method or kit is used to detect, identify or quantify non-coding RNA in a sample, including, for example, microRNA (miRNA), small nucleolar RNA (snoRNAs) and spherical nucleic acids (SNAs). In one aspect, the method or kit is used for a genotyping assay. Genotyping methods are known and generally include steps of probe hybridization, probe ligation, and signal amplification, for example, using polymerase chain reaction (PCR), immobilization of the amplified product to a support surface and detection of the target analyte. In one aspect, the method or kit is used for a human genotyping assay. In another aspect, the method or kit is used for a plant genotyping assay, for example, for an agrigenomic assay. In one aspect, the method or kit is used to characterize transcriptional activity (coding and non-coding) for example, in a gene expression analysis or transcriptome analysis.

In one aspect, the method or kit can be used for multiplex analysis of microRNA (miRNA) expression. miRNAs are small noncoding RNAs (approximately 20-22 nucleotides in length) that regulate fundamental cellular processes, including, for example, cellular differentiation and proliferation, developmental timing, hematopoiesis, immune responses, apoptosis, and nervous system patterning. The human genome includes approximately 2000 genes that encode microRNAs (miRNAs). (Kawahara (2014) Human diseases caused by germline and somatic abnormalities in microRNA and mciro-RNA related genes. Congenital Anomalies. 54:12-21). Alterations in miRNA levels, timing of expression, location or target recognition can have devastating consequences and expression profiling of miRNAs can provide valuable information regarding various biological processes. The analysis of primary, precursor, and mature miRNA levels as well as the identification and characterization of miRNA targets can be important for determining the step in miRNA biogenesis or function in a particular mutant or disease. (See, Van Wynsberghe et al. (2011) Analysis of microRNA Expression and Function. Methods Cell Biol. 106:219-252). Sequence length variability of miRNAs (isomiRs) can result in altered targeting capacity or specificity. (Cammaerts et al. (2015) Genetic variants in microRNA genes: impact on microRNA expression, function, and disease. Front. Genet. 6:186).

Various human diseases, including developmental abnormalities and cancers, are caused by either germline or somatic mutations in miRNA genes, or in miRNA-associated genes that encode the miRNA processing machinery or within miRNA-binding sites in the 3′UTRs of target mRNAs. miRNA and miRNA-related genes associated with human disease including, but not limited to, DGCR8 (DiGeorge syndrome), DICER1 (pleuropulmonary blastoma, cystic nephroma, ovarian Sertoli-Leydig-type tumors, pineoblastoma, nonepithelial ovarian tumors), TARBP2 (colon tumors, gastric tumors), XPO5 (colon tumors, gastric tumors, endometrial tumors), mR-14 and miR-146 (5q-syndrome), mi-R-17, miR-18a, miR-19a, miR-19b, miR-20a, and miR-92a (Feingold syndrome 2), miR15a and miR-16-1 (chronic lymphocytic leukemia, diffuse large B-cell lymphoma, multiple myeloma, prostate tumors), miR-16-1 (chronic lymphocytic leukemia), miR-96 (severe deafness), miR-84 (EDICT syndrome), SLITRK1 (Tourette’s syndrome), IRGM (Crohn’s disease) and HDAC6 (X-linked dominant chondrodysplasia). (Kawahara, Y. (2014) Human diseases caused by germline and somatic abnormalities in microRNA and mciro-RNA related genes. Congenital Anomalies. 54:12-21.)

In one aspect, the method or kit is used to identify, detect or quantify one or more target miRNA sequences in a sample. In one aspect, the method or kit is used to identify, detect or quantity microRNA with single base nucleotide differences. In one aspect, the method includes the use of one or more labeled probes that include a tag sequence complementary to an immobilized capture oligonucleotide sequences and a sequence complementary to the miRNA sequence. In one aspect, the label includes a biotin label. In another aspect, the label includes a chemiluminescent label. In one aspect, the method includes contacting a support surface having one or more immobilized capture oligonucleotides with one or more probes that include a tag sequence that is complementary to an immobilized capture oligonucleotide sequence and a sequence that is complementary to a target miRNA sequence under conditions suitable for binding of the tag sequence to the capture oligonucleotide sequence. The support surface is then washed to remove excess probe and is then contacted with a sample that includes or is suspected of including one or more target miRNA sequences under conditions in which the miRNA sequences is able to hybridize to the immobilized probe sequence.

In one aspect, the method or kit is used to identify, detect, or quantify one or more nucleotide sequences or variants associated with a disorder or disease, including, but not limited to, cancer, Alzheimer’s disease, cystic fibrosis, sickle cell anemia, Duchenne muscular dystrophy, thalassemia, or Huntington’s disease. In one aspect, the method or kit can be used to detect one or more polymorphisms of a polymorphic gene such as cytochrome p450.

Many diseases are known to be associated with genetic variations, including, but not limited to, hepatolenticular degeneration (APP7B), obesity (MC4R), Diabetes mellitus, type 2 (IRS1), cystic fibrosis (CTFR), Rett syndrome (MECP2), Alzheimer’s (APP), Creutzfeldt-Jakob syndrome (PRNP), Familial Mediterranean fever (MEFV), gastrointestinal stromal tumors (KIT), pheochromocytoma (RET), Duchenne muscular dystrophy (DMD), diabetes insipidus, neurogenic (AVP), fragile X syndrome (FMR1), ornithine carbamoyltransferase deficiency disease (OTC), Brugada syndrome (SCN5A), Marfan syndrome (FBN1), polycythemia vera (JAK2), polycystic kidney, autosomal recessive (PKHD1), malignant hyperthermia (RYR1), and Canavan disease (ASPA). Pinero et al. (2015) DisGeNET: a discovery platform for the dynamical exploration of human diseases and their genes. Database: doi: 10.1093/database/bav028.

In one aspect, a method or kit is provided to detect, identify or quantify one or more SNPs associated with infectious disease phenotypes, including, for example, Crutzfeldt-Jakob disease (PRNP), Dengue shock Syndrome (MICB), hepatitis B (HLA-DPA1 and HLA-DPB1); hepatitis C (IL28B); HIV-1 and AIDS (HLA-C, HLA-B, HCP5, MICA, PSORS1C3, ZNRD1, RNF39, PARD3B, and CXCR6); leprosy (LACC1, NOD2, RIPK2, CCDC122, and TNFSF15); meningococcal disease (CFH), malaria (HBB); and tuberculosis (GATA6, TAGE1, RBBP8 and CABLES1). Fareed and Afzal (2012) Single nucleotide polymorphism in genome-wide association of human population: A tool for broad spectrum science. Egypt. J. Med. Human Genet. 14:123-134.

In one aspect, a method or kit is provided to detect, identify or quantify one or more SNPs associated with a disease, including, for example, autoimmune diseases, cardiovascular conditions, diabetes, gastrointestinal disorders, lipid metabolism disorders and neuropsychiatric conditions. SNPs associated with autoimmune diseases are known and include, for example SNP associated with rheumatoid arthritis (SPRED2, ANKRD55, IL6ST, PXK, RBPJ, CCR6, IRF5, TRAF1-C5, chromosome 6q23.3 near NTAFIP3, and OLIG3) and systemic lupus erythematosus (BANK1). SNPs associated with cardiovascular conditions are known and include, for example SNP associated with atrial fibrillation/atrial flutter (chromosome 4q25 near PITX2); coronary disease (CDKN2A/B, and MTHFD1L), coronary heart disease (DAB2IP); and myocardial disease (CDKN2A/B). SNPs associated with diabetes are known and include, for example SNP associated with Type 1 diabetes (FUT2, C12orf30, ERBB3, KIAA0350, PTPN2, CD226, TRAFD1, and PTPN11); and Type 2 diabetes (KCNQ1, SLC30A8, FTO, HHEX, CDKAL1, CDKN2B, IGFBP2, CDKN2A/B, and IGF2BP2). SNP associated with gastrointestinal disorders are known and include, for example, SNP associated with celiac disease (KIA1109, TENR, IL2, and IL21); Crohn’s disease (PTPN2, IRGM, NKX2-3, ATG16L1, BSN, MSTI, and IRGM); gallstones (ABCG8 and SH2B3/LNK); and inflammatory bowel disease (IL23R). SNP associated with lipid metabolism disorders include, for example, SNP associated with HDL-cholesterol (GALNT2 and MVK/MMAB); LDLl-cholesterol (CELSR2, PSRC1, SORT1, CILP2, and PBX4); triglycerides (BCL7B, TBL2, MLXIPL, CILP2, PBX4, TRIB1, GALNT2, ANGPTL3, DOCK7, ATG4C, GCKR, TRIB1, NCAN/CILP2, and MLXIPL). SNP associated with neuropsychiatric conditions are known and include, for example, SNP associated with amyotrophic lateral sclerosis (DPP6); APOE e4 with late-onset Alzheimer disease (GAB2); bipolar disorder (DGKH, PALB2, NDUFAB1, and DCTN5); multiple sclerosis (KIAA 0350, IL2RA and IL7RA); restless leg syndrome (BTBD9, MEIS1, BTBD9, MAP2K5 and LBXCOR1); and schizophrenia (CSF2RA). Fareed and Afzal (2012) Single nucleotide polymorphism in genome-wide association of human population: A tool for broad spectrum science. Egypt. J. Med. Human Genet. 14:123-134.

In one aspect, the method or kit is used to identify, detect or quantify one or more nucleotide sequences or variants associated with cancer. In one aspect, the nucleic acid sequence is a wild-type sequence. In one aspect, the nucleic acid sequence is a mutant or variant sequence. In one aspect, a mutation in the nucleotide sequence is associated with cancer. In one aspect, the method or kit is used to identify, detect or quantify the presence or absence of a wild-type, mutant or variant nucleic acid sequence for one or more oncogenes or proto-oncogenes, such as BRAF or KRAS, or one or more tumor suppressor genes, such as BRCA1, BRCA2, PTEN, CTFR and TP53, and combinations thereof. (See, for example, Concert Genetics (2017) The Current Landscape of Genetic Testing).

In one aspect, a method or kit is used to detect medically relevant DNA- or RNA-based markers for cancer. In another aspect, the method or kit is used to personalize medicine to assist in the selection of an effective cancer therapy. In one aspect, the method or kit is used to identify persons at-risk for a hereditary cancer. Hereditary cancer refers to a group of genetic defects which significantly elevate the risk of a person developing cancer which can be can be diagnosed by the identification of germ-line mutations in specific genes, including for example, Li-Fraumeni syndrome (p53), familial adenomatous polyposis (APC), breast cancer (BRCA1; BRCA2; PALB2; TP53; CHEK2; ATM; NBS/NBN; BLM; PTEN; MRE11; BRIP1; BARD1; RAD50; RAD51C; RAD51D; RECQL; FANCC; and FANCM), and hereditary non-polyposis colorectal cancer (HNPCC) syndrome (MLH1; MSH2; MSH3; MSH6; PMS2; EPCAM; APC; MUTYH; NTHL1; POLE; POLD1; SMAD4; BMPRIA;and STK11). Sokolenko and Imyanitov (2018) Molecular Diagnostics in Clinical Oncology. Front. Molec. Bio. 5(76): 1-15.

Additional SNP markers for cancers are known and include markers for, for example, breast cancer (FGFR2, TNCR9/LOC643714, MAP3K1, LSP1, and ERBB4); basal cell carcinoma (RHOU, PADI4, PADI6, RCC2, ARHGEFIOL, KRT5, CDKN2A/B, TCF2, IGF2, IGF2A, INS and TH); colorectal cancer (ORF, DQ515897 and SMAD7); lung cancer (CHRNA3, CHRNA5, CHRNB4, PSMA4, LOCI23688 and TRNAA-UGC); melanoma (CDC91L1), neuroblastoma (FLJ22536, FLJ44180, and BARD1); and thyroid cancer (FOXE1 and NKX2-1). Fareed and Afzal (2012) Single nucleotide polymorphism in genome-wide association of human population: A tool for broad spectrum science. Egypt. J. Med. Human Genet. 14:123-134.

In one aspect, a method or kit is provided to detect, identify or quantify one or more copy number variants (CNV) or aneuploidy associated with human disease, including, for example, neurodevelopmental disorders such as autism, intellectual disability and epilepsy, congenital heart defects and other congenital anomalities. In one aspect, the CNV includes a deletion. In another aspect, the CNV includes a duplication. Examples of disorders associated with a deletion CNV include, but are not limited to, disorders affecting head size, psychiatric disorders and metabolism (KCTD13 and PRRT2), sleep regulation and metabolism (RAI1), facial appearance (ELN), cardiac abnormalities, infantile hypercalcemia, growth or developmental delay (LIMK-1), dysmorphic features, developmental delay, heart defects (GATA4), intellectual disability, epilepsy, seizures, dysmorphism of face and digits (CHRNA7), intellectual disability, distinctive facial features, epilepsy, heart defects, urogenital anomalities (KANSL1), and dysmorphic facial features, velocardio-facial syndrome, cogenital heart disease, learning disabilities, hearing loss (TBX1). Examples of disorders associated with a duplication CNV include, but are not limited to, disorders affecting head size, psychiatric disorders and metabolism (KCTD13 and PRRT2), sleep regulation and metabolism (RAI1), facial appearance (ELN), dysmorphic features, developmental delay, heart defects (GATA4), language and speech delay, autism, epilepsy (LIMK-1), intellectual disability, autism, recurrent ear infections, low set ears, obesity (CHRNA7), developmental delay, microcephaly, facial dysmorphism, abnormal digits and hirsutism, failure to thrive (KANSL1), and dysmorphic facial features, velopharyngeal insufficiency, congenital heart disease, intellectual disabilities, speech delay, hearing loss and failure to thrive (TBX1). Golzio and Katsanis (2013) Genetic Architecture of Reciprocal CNVs. Curr. Opin. Genet. Dev. 23(3):240-248. Frequently observed disorders associated with CNVs include, but are not limited to, Willaims (ELN, deletion phenotype), Prader-Willi or Angelman (UBE3A, deletion phenotype), Smith-Magenis (RAI1, deletion phenotype), Potocki-Lupski (RAI1, duplication phenotype), Koolen-de Vries (MAPT, KANSL1, deletion phenotype), DiGeorge/Velo-cario-facial (TBX1, HIRA, deletion phenotype), and renal cysts and diabetes (HNFIB, deletion phenotype). Martin et al. (2015) CNVs, Aneuploidies and Human Disease. Clinics and Perinatology. 42(2):227-242, see also, Aouiche et al. (2018) Copy number variation related disease genes. Quant. Biol. 6(2):99-112.

In one aspect, the method or kit described herein can be used as a companion diagnostic device to provide information relating to the use of a corresponding therapeutic product. For example, the method or kit can be used to detect, identify or quantify one or more genes, such as, BRCA1 or BRCA2 for patient management relating to therapeutics such as Lynparza® (olaparib), Talzenna®(talazoparib), or Rubraca® (rucaparib) for breast or ovarian cancer; EGFR for patient management relating to therapeutics such as Iressa® (gefitinib), Gilotrif® (afatinib) or Vizimpro® (dacomitinib), Tarceva® (eroltinib), or Tagrisso® (osimertinib) for non-small cell lung cancer; PD-L1 for patient management relating to therapeutics such as Keytruda® (pembrolizimab) or Tecentriq® (atezolizumab) for non-small cell lung cancer; IDH1 for patient management relating to therapeutics such as Tibsovo® (ivosidenib) for acute myeloid leukemia; BCR-ABL for patient management relating to therapeutics such as Tasigna® (nilotinib) for chronic myeloid leukemia; ALK for patient management relating to therapeutics such as Zykadia® (ceritinib), Xalkori® (crizotinib), and Alecensa® (alectinib) for non-small cell lung cancer; IDH2 for patient management relating to therapeutics such as Idhifa®(enasidenib) for acute myeloid leukemia; RAS for patient management relating to therapeutics such as Vectibix®(panitumumab) for colorectal cancer; FLT3 for patient management relating to therapeutics such as Rydapt®(midostaurin) and Xospata®(gilterinib) for acute myelogenous leukemia; KIT (D816V) for patient management relating to therapeutics such as Gleevec® (imatinib mesylate) for aggressive systemic mastocytosis; PDGFRB for patient management relating to therapeutics such as Gleevec® (imatinib mesylate) for myelodysplastic syndrome/myeloproliferative disease; KRAS or EGFR for patient management relating to therapeutics such as Erbitux® (cetuximab) or Vectibix® (panitumumab) for colorectal cancer; c-KIT for patient management relating to therapeutics such as Gleevec® (imatinib mesylate) or Glivec® (imatinib mesylate) for gastrointestinal stromal tumors; HER-2 for patient management relating to therapeutics such as Herceptin® (trastuzumab), Perjeta®(pertuzumab), or Kadcyla®(ado-trastuzumab) for breast cancer; HER-2 for patient management relating to therapeutics such as Herceptin®(trastuzumab) for gastric and gastroesophogeal cancer; BRAF for patient management relating to therapeutics such as Braftovi®(encorafenib), Mektovi®(binimetinib), Mekinist®(tramatenib), Tafinilar®(dabrafenib), Zelboraf®(vemurafenib), or Cotellic®(cobimetinib) for melanoma; or combinations thereof. See, for example, FDA List of Cleared or Approved Companion Diagnostic Devices (In Vitro and Imaging Tools) available at fda.gov.

In one aspect, the method or kit is used to identify, detect or quantify one or more nucleotide sequences or variants to detect pathogenic organisms in a clinical or environmental sample, for example, for clinical diagnostics, food safety testing, environmental monitoring or biodefense. In one aspect, the method or kit is used to identify, detect or quantify one or more pathogens including, viral, bacterial, parasitic and fungal pathogens. In one aspect, the method or kit is used to identify, detect or quantify one or more antibiotic or antiviral resistant pathogenic organisms.

In one aspect, the method or kit includes one or more sets of probes configured to detect the presence of one or more pathogenic genomes. In one aspect, the method or kit is used for high-throughput screening for pathogen detection, genotyping, detection of viruses, detection of virulence markers, detection of antibiotic resistance or outbreak investigation, see, for example, Fourier et al. (2014) Clinical Detection and Characterization of bacterial pathogens in the genomics era. Genome Medicine. 6:114. In one aspect, a method or kit is provided for detection and genotyping of viral pathogens, see, for example, Wang et al. (2002) Microarray-based detection and genotyping of viral pathogens. PNAS. 99(24):15687-15692.

Viral genomes sequences are known and can be found, for example, using the NCBI Viral Genomes Resource, which catalogs all publicly available virus genome sequences and can be accessed at ncbi.nlm.nih.gov/genome/viruses. Similarly, microbial genome sequences are known and can be found, for example, using the NCBI Microbial Genome Resource, which catalogs all publicly available microbial genome sequences and can be accessed at ncbi.nlm.nih.gov/genome/microbes.

In another aspect, the method or kit can be used to detect, identify or quantify one or more viruses, for example, one or more respiratory viruses including, but not limited to, influenza A and B viruses, including for example, influenza A virus subtypes H1, H3, and H5; parainfluenza virus types 1, 2, 3, and 4; respiratory syncytial virus types A and B; adenovirus; metapneumovirus (MPV); rhinovirus; enterovirus; and coronaviruses (CoV) such as OC43 and 229E or severe acute respiratory syndrome coronavirus, NL63, and HKU1; avian influenza virus H5N1; and human bocavirus. In one aspect, a method or kit is provided for detecting the viral capsid (CA) protein.

In one aspect, the method or kit includes one or more “discovery” probes that match genome regions that are unique to a taxonomic family or subfamily, but are shared by the species within that family. “Discovery” probes target sequences that evolve more slowly within families and are useful for detecting species within a known family. In another aspect, the method or kit includes one or more “census” probes that target highly variable regions that are unique to an individual species or strain. “Census” probes are useful for identifying the specific strain of organism in a sample. McLoughlin, K.S. (2011) Microarrays for Pathogen Detection and Analysis. Brief Funct. Genomics. 10(6):342-353.

In one aspect, the method or kit are used to detect, identify or quantify a nucleic acid sequence associated with a pathogenic bacteria. A common gene target used to identify a wide variety of aerobic and anaerobic bacteria is 16S rRNA or rDNA. The rpoB gene, which encodes the β-subunit of bacterial RNA polymerase can also used for bacterial identification, for example, for the identification of rapidly growing mycobacteria. Other bacterial gene targets include tuf (elongation factor Tu), gyrA or gyrB (gyrase A or B), soda (manganese-dependent superoxide dismutase) and heat shock proteins. Petti, C.A. (2007) Detection and Identification of Microorganisms by Gene Amplification and Sequencing. Clin. Infect. Dis. 44:1108-1114.

In one aspect, the method or kit is used to identify, detect or quantify one or more pathogenic organisms in a stool specimen. In one aspect, the method or kit is used to identify, detect or quantify one or more viral, parasitic or bacterial nucleic acid sequences in a human stool specimen. In one aspect, the method or kit is used to identify, detect or quantify one or more bacteria or bacterial toxins, including, but not limited to Campylobacter, Clostridium dificile toxin A/B, Escherichia coli 0157, enterotoxin E. coli (ETEC) LT/ST, shiga-like toxin producing E. coli (STEC) stx1/stx2, Salmonella, Shigella, Vibrio cholerae, and Yersinia enterocolitica. In one aspect, the method or kit is used to identify, detect or quantify one or more viruses, including, but not limited to, adenovirus, norovirus and rotavirus. In one aspect, the method or kit is used to identify, detect or quantify one or more parasites, including, but not limited to, Cryptosporidium, Entamoeba hisolytica, or Giardia.

In one aspect, the method or kit is used to identify, detect or quantify one or more nucleotide sequences or variants associated with organ transplantation outcomes. In one aspect, the method or kit is used to identify, detect or quantify human leukocyte antigen (HLA) to provide information helpful for organ transplantation procedures. Human leukocyte antigen (HLA) molecules are expressed on almost all nucleated cells and are important in graft rejection. The system is highly polymorphic. There are three classical loci at HLA class I: HLA-A, -B, and -Cw, and five loci at class II: HLA-DR, -DQ, -DP, -DM, and -DO. Mahdi, B.M. (2013) A glow of HLA typing in organ transplantation. Clin. Transl. Med. 2:6. Over 7,500 different alleles and over 5,458 expressed antigens are currently known. (Laperrousaz et al. (2012) HLA and non-HLA polymorphisms in renal transplantation. Swiss Med. Wkly. 142:w13668.

In one aspect, the method or kit is used to identify, detect or quantify nucleic acids, for example, nucleic acid therapeutics, in a patient’s circulation. A variety of nucleic acid therapeutics are known and include DNA therapeutics such as antisense oligonucleotides, DNA aptamers and gene therapy, and RNA therapeutics such as microRNAs, short interfering RNAs, ribozymes, RNA decoys and circular RNAs. Examples of antisense oligonucleotides include Fomivirsen, for the management of cytomegalovirus (CMV) retinitis and Mipomersen, an inhibitor of apolipoprotein B-100 synthesis. Examples of oligonucleotides used in gene therapy include Gendicine, for the expression of tumor suppressor gene p53 and Alipgene, for patients with lipoprotein lipase deficiency. Miravirsen is an antisense oligonucleotide that targets liver-specific microRNA-122. Additional therapeutic nucleic acids in clinical trials are listed in Sridharan and Gogtay (2016) Therapeutic Nucleic Acids: Current Clinical Status. Br. J. Clin. Pharmacol. 82(3):659-672, the disclosure of which is incorporated herein in its entirety.

In one aspect, a method or kit is provided for gene expression studies. In one aspect, a method or kit is provided to detect, identify or quantify mRNA expression in a sample. In one aspect, a method or kit is provided to detect, identify or quantify one or more regulatory polymorphisms (rSNP). The term “regulatory polymorphism” refers to a polymorphism that occurs outside an exonic region that can impact gene expression. A cis-acting regulatory polymorphism acts on a copy of a gene present on the same allele and, is typically present in or near the locus of the gene that it regulates. A trans-acting regulatory polymorphism is a polymorphism in one gene that affects the expression of another gene. Knight, J.C. (2005) Regulatory Polymorphisms underlying complex disease traits. J. Mol. Med. (Berl.). 83(2):97-109. Cis- and trans-acting polymorphic regulators for human genes are known and include those described by Cheung et al. (2010) Polymorphic Cis- and Trans-Regulation of Human Gene Expression. PLOS Biol. 8(9):e1000480, the disclosure of which is incorporated by reference herein in its entirety.

In one aspect, the method or kit is used to identify, detect or quantify one or more nucleotide sequences or variants, such as DNA methylation polymorphisms or other epigenetic variations.

In one aspect, the method or kit is used to identify, detect, or quantify microsatellite instability (MSI). MSI is indicative of a predisposition to mutation resulting from impaired DNA mismatch repair. MSI is further described in, e.g., Schlotterer et al., “Microsatellite Instability,” eLS 2004; doi:10.1038/npg.els.0000840.

In one aspect, the method or kit is used to identify, detect, or quantify one or more nucleotide sequences or variants due to gene editing technology, including, for example, clustered regularly interspaced short palindromic repeat (CRISPR), transcription activator-like effector nuclease (TALEN), and zinc finger nucleases (ZFN).

In one aspect, the method or kit is used to identify, detect or quantify one or more proteins in a sample. In one aspect, the protein is a DNA binding protein. In one aspect, the method or kit is used to isolate one or more target DNA binding proteins from a sample. In another aspect, the method or kit is used to confirm the identity of one or more DNA binding proteins in a sample or determine the relative amount of DNA binding proteins in a sample. In one aspect, the method or kit is used to measure transcription factor-DNA binding interaction. In one aspect, a single stranded or double stranded DNA sequence to which a DNA binding protein bind is immobilized to a support surface as described herein and contacted with a sample that contains or is suspected of containing a DNA binding protein. In one aspect, the immobilized DNA sequence is contacted with the sample that contains or is suspected of containing the DNA binding protein under conditions in which the DNA binding protein binds to the immobilized DNA sequence on the support surface. The surface is then washed to remove debris, including, for example, non-specifically bound protein. In one aspect, the target DNA binding protein is eluted from the immobilized DNA and detected, for example, by western blot or mass spectrometry. In another aspect, the immobilized target DNA binding protein is labeled and detected, for example, using a labeled antibody that specifically binds to the protein or an electrochemiluminescent label. In one aspect, the sample is a cell lysate that includes one or more DNA binding proteins. In one aspect, the support surface is a microwell plate. In one aspect, the microplate format is used in connection with a high-throughput analysis, for example, for mutational or activation assays.

In one aspect, the method or kit is used to identify, detect or quantify one or more nucleotide sequences or variants, such as single nucleotide variants or single nucleotide polymorphisms associated with pathogenicity or drug resistance. In another aspect, the method or kit is used to identify, detect or quantify one or more nucleotide sequences or variants, such as single nucleotide variants or single nucleotide polymorphisms associated with a specific industrial or agriculture application, for example, mutations associated with a genetic modified organism (GMO). In one aspect, the method or kit can be used in a genome wide association studies (GWAS) to determine whether one or more variants, for example, single nucleotide variants, are associated with a disease.

In one aspect, the method or kit is used to identify, detect, or quantify one or more single nucleotide variants. In one aspect, the method or kit is used to identify, detect, or quantify between about 1 and about 100, or about 5 and about 100 defined single nucleotide variants, which can include one or more single nucleotide polymorphisms.

In one aspect, methods and kits are provided for simultaneous, parallel identification, detection or quantification of a plurality of target nucleotides sequences in a sample. In one aspect, a method is provided for identifying, detecting or quantifying up to 100 target nucleotide sequences in a sample, for example, between about 1 and about 100, or about 5 and about 100 target nucleotide sequences in a sample. In one aspect, a method or kit is provided in which a user or manufacturer can configure a multiplexed binding assay for detecting one or more target nucleotide sequences based on specific user requirements.

In one aspect, the method includes generating a tagged and labeled reaction product using a target nucleotide sequence as a template and contacting a support surface with the tagged and labeled reaction product, wherein the support surface includes patterned arrays of one or more binding domains to which a plurality of capture molecules are immobilized. In one aspect, the capture molecules include single stranded capture oligonucleotides immobilized on discrete binding domains, in which each binding domain includes capture oligonucleotides having a particular nucleotide sequence. In one aspect, the tagged and labeled reaction product includes a single stranded oligonucleotide tag having a sequence complementary to the sequence of a capture oligonucleotide. In one aspect, the tagged and labeled reaction product is generated by an oligonucleotide ligation assay (OLA). In another aspect, the tagged and labeled reaction product is generated by a primer extension assay (PEA). In one aspect, the label is an electrochemiluminescent (ECL) label and the support surface includes one or more working electrodes and one or more counter electrodes suitable for triggering an electrochemiluminescent emission from a label of an immobilized reaction product.

In one aspect, the target nucleotide sequence includes or is suspected of containing a wild-type sequence. In one aspect, the target nucleotide sequence includes or is suspected of containing a mutation, such as a deletion, addition, substitution, transition, transversion, rearrangement, or translocation. In one aspect, the mutation includes a missense, nonsense, silent, or splice-site mutation. In one aspect, methods and kits are provided for identifying, detecting or quantifying one or more single nucleotide polymorphisms (SNPs) in one or more target nucleotide sequences. In one aspect, methods and kits are provided for identifying, detecting or quantifying one or more common single nucleotide SNPs that are present in at least about 1% of the population. In another aspect, methods and kits are provided for identifying, detecting or quantifying mutations that are present at a low frequency in a sample, for example, mutations present at less than 0.05% or 0.01% in a sample.

In one aspect, a method of conducting a multiplexed binding assay for a plurality of target analytes is provided. Multiplex binding assays are known and include those described in U.S. Pat. Publication No. 2016/0069872, filed Sep. 8, 2015, entitled METHODS FOR CONDUCTING MULTIPLEXED ASSAYS, the disclosure of which is incorporated herein in its entirety.

In one aspect, the method of conducing a multiplexed binding assay includes providing a support surface on which at least a first capture oligonucleotide having a first nucleotide sequence is immobilized on a first binding domain and a second capture oligonucleotide having a second nucleotide sequence is immobilized on a second binding domain. In one aspect, the first and second nucleotide sequences are not the same. In one aspect, the support surface is contacted, in one or more steps, with at least a first targeting agent, a first binding reagent, a second targeting agent and a second binding reagent. In one aspect, the first targeting agent includes a first tag sequence operably connected to a first linking agent. In one aspect, the first tag sequence includes a nucleotide sequence that is complementary to the nucleotide sequence of the first capture oligonucleotide. In one aspect, the second targeting agent includes a second tag sequence operably connected to a second linking agent. In one aspect, the second tag sequence includes a nucleotide sequence that is complementary to the nucleotide sequence of the second capture oligonucleotide. In one aspect, the first binding reagent includes a first analyte binding domain specifically binds to a first analyte operably connected to a first supplemental linking agent. In one aspect, the second binding reagent includes a second analyte binding domain that specifically binds to a second analyte operably connected to a second supplemental linking agent. In one aspect, the first linking agent is a binding partner of the first supplemental linking agent and the second linking agent is a binding partner of the second linking agent. In one aspect, the support surface is contacted with at least a first and a second bridging agent. In one aspect, the first bridging agent includes a first linking agent binding site that binds to the first linking agent and a first supplemental linking agent binding site that binds to the first supplemental linking agent and the second bridging agent includes a second linking agent binding site that binds to the second linking agent and a second supplemental linking agent binding site that binds to the second supplemental linking agent.

In one aspect, the support surface is contacted with a sample that contains or is suspected of containing at least a first analyte of interest and a second analyte of interest. In one aspect, at least a first binding complex and a second binding complex are formed. In one aspect, the first binding complex is formed on the first binding domain and includes the first targeting agent, the first capture oligonucleotide, the first binding reagent and the first analyte. In one aspect, the first binding complex is formed on the first binding domain and includes the first targeting agent, the first capture oligonucleotide, the first bridging agent, the first binding reagent and the first analyte. In one aspect, the second binding complex is formed on the second binding domain and includes the second targeting agent, the second capture oligonucleotide, the second binding reagent and the second analyte. In one aspect, the second binding complex is formed on the second binding domain and includes the second targeting agent, the second capture oligonucleotide, the second bridging agent, the second binding reagent and the second analyte. In one aspect, the method includes measuring the amount of first and second analytes immobilized on the first and second binding domains, respectively, via the first and second binding complexes.

### O. Manual and Automated Embodiments

Methods disclosed herein may be performed manually, using automated technology, or both. Automated technology may be partially automated, e.g., one or more modular instruments, or a fully integrated, automated instrument.

Example automated systems are discussed and described in commonly owned International Patent Appl. Pub. Nos. WO 2018/017156 and WO 2017/015636 and International Patent Appl. Pub. No. WO 2016/164477, each of which is incorporated by reference in its entirety.

Automated systems (modules and fully integrated) on which the methods herein may be carried out may include the following automated subsystems: computer subsystem(s) that may include hardware (e.g., personal computer, laptop, hardware processor, disc, keyboard, display, printer), software (e.g., processes such as drivers, driver controllers, and data analyzers), and database(s); liquid handling subsystem(s), e.g., sample handling and reagent handling, e.g., robotic pipetting head, syringe, stirring apparatus, ultrasonic mixing apparatus, magnetic mixing apparatus; sample, reagent, and consumable storing and handling subsystem(s), e.g., robotic manipulator, tube or lid or foil piercing apparatus, lid removing apparatus, conveying apparatus such as linear and circular conveyors and robotic manipulators, tube racks, plate carriers, trough carriers, pipet tip carriers, plate shakers; centrifuges, assay reaction subsystem(s), e.g., fluid-based and consumable-based (such as tube and multi well plate); container and consumable washing subsystem(s), e.g., plate washing apparatus; magnetic separator or magnetic particle concentrator subsystem(s), e.g., flow cell, tube, and plate types; cell and particle detection, classification and separation subsystem(s), e.g., flow cytometers and Coulter counters; detection subsystem(s) such as colorimetric, nephelometric, fluorescence, and ECL detectors; temperature control subsystem(s), e.g., air handling, air cooling, air warming, fans, blowers, water baths; waste subsystem(s), e.g., liquid and solid waste containers; global unique identifier (GUI) detecting subsystem(s) e.g., 1D and 2D bar-code scanners such as flat bed and wand types; sample identifier detection subsystem(s), e.g., 1D and 2D bar-code scanners such as flat bed and wand types. Analytical subsystem(s), e.g., chromatography systems such as high-performance liquid chromatography (HPLC), fast-protein liquid chromatography (FPLC), and mass spectrometer can also be modules or fully integrated.

Systems or modules that perform sample identification and preparation may be combined with (or be adjoined to or adjacent to or robotically linked or coupled to) systems or modules that perform assays and that perform detection or that perform both. Multiple modular systems of the same kind may be combined to increase throughput. Modular system(s) may be combined with module(s) that carry out other types of analysis such as chemical, biochemical, and nucleic acid analysis.

The automated system may allow batch, continuous, random-access, and point-of-care workflows and single, medium, and high sample throughput.

The system may include, for example, one or more of the following devices: plate sealer (e.g., Zymark), plate washer (e.g., BioTek, TECAN), reagent dispenser and/or automated pipetting station and/or liquid handling station (e.g., TECAN, Zymark, Labsystems, Beckman, Hamilton), incubator (e.g., Zymark), plate shaker (e.g., Q.Instruments, Inheco, Thermo Fisher Scientific), compound library or sample storage and/or compound and/or sample retrieval module. One or more of these devices is coupled to the apparatus of the invention via a robotic assembly such that the entire assay process can be performed automatically. According to an alternate embodiment, containers (e.g., plates) are manually moved between the apparatus and various devices (e.g., stacks of plates).

The automated system may be configured to perform one or more of the following functions: (a) moving consumables such as plates into, within, and out of the detection subsystem, (b) moving consumables between other subsystems, (c) storing the consumables, (d) sample and reagent handling (e.g., adapted to mix reagents and/or introduce reagents into consumables), (e) consumable shaking (e.g., for mixing reagents and/or for increasing reaction rates), (f) consumable washing (e.g., washing plates and/or performing assay wash steps (e.g., well aspirating)), (g) measuring ECL in a flow cell or a consumable such as a tube or a plate. The automated system may be configured to handle individual tubes placed in racks, multiwell plates such as 96 or 384 well plates.

Methods for integrating components and modules in automated systems as described herein are well-known in the art, see, e.g., Sargeant et al., Platform Perfection, Medical Product Outsourcing, May 17, 2010.

In embodiments, the automated system is fully automated, is modular, is computerized, performs in vitro quantitative and qualitative tests on a wide range of analytes and performs photometric assays, ion-selective electrode measurements, and/or electrochemiluminescence (ECL) assays. In embodiments, the system includes the following hardware units: a control unit, a core unit and at least one analytical module.

In embodiments, the control unit uses a graphical user interface to control all instrument functions, and is included of a readout device, such as a monitor, an input device(s), such as keyboard and mouse, and a personal computer using, e.g., a Windows operating system. In embodiments, the core unit is included of several components that manage conveyance of samples to each assigned analytical module. The actual composition of the core unit depends on the configuration of the analytical modules, which can be configured by one of skill in the art using methods known in the art. In embodiments, the core unit includes at least the sampling unit and one rack rotor as main components. Conveyor line(s) and a second rack rotor are possible extensions. Several other core unit components can include the sample rack loader/unloader, a port, a barcode reader (for racks and samples), a water supply and a system interface port. In embodiments, the analytical module conducts ECL assays and includes a reagent area, a measurement area, a consumables area and a pre-clean area.

### P. Kits

In one aspect, a kit is provided for conducting an assay to identify, detect or quantify one or more target analytes in a sample. In one aspect, the kit can be customized, by the manufacturer or the end user, to identify, detect or quantify one or more target proteins or nucleotide sequences of interest. In one aspect, the end user can designate which target analyte will be directed to each binding domain in an array based on the complementarity between the oligonucleotide tag associated with the target analyte or reaction product and the capture oligonucleotide immobilized in each binding domain. In one aspect, the kit provides a multi-well assay plate that can be configured based on a user’s specifications, e.g., an end-user can select a set of analytes and configure a user-customized multiplexed assay for that set of analytes.

In one aspect a kit is provided. In one aspect, the kit includes a set of non-cross-reactive capture oligonucleotides as described herein. In one aspect, the kit includes two or more non-cross-reactive capture oligonucleotides selected from Table 1 (SEQ ID NOs: 1-64), Table 2 (SEQ ID NOs: 65-122), Table 3 (SEQ ID NOs: 123-186), Table 4 (SEQ ID NOs: 187-250), Table 5 (SEQ ID NOs: 251-308), Table 6 (SEQ ID NOs: 309-372), Table 7 (SEQ ID NOs: 373-436), Table 8 (SEQ ID NOs: 437-494), Table 9 (SEQ ID NOs: 495-558), Table 10 (SEQ ID NOs: 559-622), Table 11 (SEQ ID NOs: 623-680), or Table 12 (SEQ ID NOs: 681-744), or variants thereof. In one aspect, the kit includes a set of two or more non-cross-reactive capture oligonucleotides selected from SEQ ID Nos: 1-64, or variants thereof. In one aspect, the kit includes a set of two or more non-cross-reactive capture oligonucleotides selected from SEQ ID Nos: 1-10, or variant thereof. In one aspect, the capture oligonucleotide includes at least 24, 30 or 36 nucleotides.

In one aspect, the kit includes a set of at least 2, 3, 4, 5, 6, 7, 8, 9, 10, 11, 12, 13, 14, 15, 16, 17, 18, 19, 20, 21, 22, 23, 24 or 25 and up to 64 non-cross-reactive capture oligonucleotides. In one aspect, the kit includes a set of up to 10 non-cross-reactive capture oligonucleotides.

In one aspect, the kit includes one or more capture oligonucleotides provided in containers, wherein the capture oligonucleotides in a container have the same sequence and each container contains capture oligonucleotides having a sequence different from (and not complementary to) the sequence of the capture oligonucleotides in the other containers. In one aspect, the kit includes, in separate containers, at least 2, 3, 4, 5, 6, 7, 8, 9, 10, 11, 12, 13, 14, 15, 16, 17, 18, 19, 20, 21, 22, 23, 24 or 25 and up to 64 non-cross-reactive capture oligonucleotides. In one aspect, the kit includes, in separate containers, up to 10 different capture oligonucleotides that can be used to identify, detect or quantify up to 10 target nucleotide sequences.

In one aspect, the kit includes a support surface and a set of non-cross-reactive capture oligonucleotides as described herein. In one aspect, the kit includes a set of non-cross-reactive capture oligonucleotides immobilized on a support surface. In one aspect, the kit includes a set of non-cross-reactive capture oligonucleotides immobilized on a support surface in an array. In one aspect, the kit includes one or more capture oligonucleotides immobilized to one or more discrete binding domains with a known location within an array. In one aspect, the kit includes two or more non-cross-reactive capture oligonucleotides immobilized on a bead array.

In one aspect, the kit includes one or more non-cross-reactive capture oligonucleotides immobilized in one or more binding domains on a support surface. In one aspect, the kit includes two or more non-cross-reactive capture oligonucleotides immobilized in two or more unique binding domains, wherein the sequence of capture oligonucleotides immobilized on each unique binding domain are the same. In one aspect, the kit includes one or more binding domains in which at least some capture oligonucleotides are not covalently bound to the support surface. In one aspect, the kit includes one or more binding domains in which at least some capture oligonucleotides are not covalently bound to the carbon-based surface, for example, carbon-based electrode, through a thiol group. In one aspect, one or more binding domains include more than 10%, 15%, 20%, 25%, 50% or 75% capture oligonucleotides that are not covalently bound to the support surface through a thiol group. In one aspect, the kit includes one or more binding domains having less than about 0.1%, 0.09%, 0.08%, 0.07%, 0.06%, 0.05%, 0.04%, 0.03%, 0.02%, or 0.01% contaminating capture oligonucleotides.

In one aspect, the kit includes one or more capture oligonucleotides that include a functional group. In one aspect, the kit includes one or more capture oligonucleotides that include a thiol group. In one aspect, one or more capture oligonucleotides are covalently attached to a carbon-based support surface through the thiol group. In one aspect, one or more capture oligonucleotides are attached to the thiol group through a linker. In one aspect, one or more capture oligonucleotides are attached to one or more electrodes through a thiol group.

In another aspect, the kit includes a set of non-cross-reactive oligonucleotide tags as described herein. In one aspect, the kit includes a set of non-cross-reactive oligonucleotide tags that bind to a non-complementary capture oligonucleotide less than 0.05% relative to a complementary capture oligonucleotide.

In one aspect, the kit includes a set of non-cross-reactive oligonucleotides selected from Table 13 (SEQ ID NOs: 745-808), Table 14 (SEQ ID NOs: 809-866), Table 15 (SEQ ID NOs: 867-930), Table 16 (SEQ ID NOs: 931-994), Table 17 (SEQ ID NOs: 995-1052), Table 18 (SEQ ID NOs: 1053-1116), Table 19 (SEQ ID NOs: 1117-1180), Table 20 (SEQ ID NOs: 1181-1238), Table 21 (SEQ ID NOs: 1239-1302), Table 22 (SEQ ID NOs: 1303-1366), Table 23 (SEQ ID NOs: 1367-1424), or Table 24 (SEQ ID NOs: 1425-1488), or variants thereof. In one aspect, the oligonucleotide tag includes at least 20, 24, 30 or 36 nucleotides.

In one aspect, the kit includes one or more oligonucleotides oligonucleotide tags provided in containers, wherein the oligonucleotide tags in a container have the same sequence and each container contains oligonucleotide tags having a sequence different from (and not complementary to) the sequence of the oligonucleotide tags in the other containers. In one aspect, the kit includes, in separate containers, at least 2, 3, 4, 5, 6, 7, 8, 9, 10, 11, 12, 13, 14, 15, 16, 17, 18, 19, 20, 21, 22, 23, 24 or 25 and up to 64 non-cross-reactive oligonucleotide tags. In one aspect, the kit includes a set of up to 10 non-cross-reactive oligonucleotide tags.

In one aspect, the kit includes a support surface. In one aspect, the kit includes a carbon-based support surface. In one aspect, the support surface includes at least one electrode. In one aspect, the electrode is a carbon-based electrode. In one aspect, the support surface includes one or more carbon ink electrodes. In one aspect, the support surface includes at least one working electrode and at least one counter electrode.

In one aspect, the kit includes a support surface that includes a multi-well assay plate. In one aspect, one or more wells of the multi-well plate include one or more electrodes. In one aspect, the support surface includes a multi-well plate wherein one or more wells include one or more working electrodes and one or more counter electrodes. In one aspect, the support surface includes one or more reference electrodes.

In one aspect, the kit includes a support surface having one or more electrodes on which one or more arrays of capture oligonucleotides are printed. In one aspect, the kit includes one or more multi-well plates on which one or more arrays of capture oligonucleotides have been printed. In another aspect, the kit includes one or more multi-well plates and one or more vials that include one or more capture oligonucleotides, wherein the capture oligonucleotides can be printed onto the multi-well plates. In one aspect, the end user or manufacturer can customize which target nucleotide sequences are identified, detected or quantified by associating an oligonucleotide tag with a target analyte or generating a reaction product having a oligonucleotide tag that is complementary to a capture oligonucleotide provided with the kit.

In one aspect, the kit includes one or more capture oligonucleotides immobilized to one or more binding domains on the support surface. In one aspect, the kit includes one or more capture oligonucleotides immobilized on one or more binding domains within a well of a multi-well plate. In one aspect, the kit includes one or more capture oligonucleotides immobilized on one or more binding domains on an electrode. In one aspect, the kit includes one or more capture oligonucleotides immobilized on one or more binding domains on an electrode within one or more wells of a multi-well plate.

In one aspect, the kit includes one or more multi-well plates in which up to 10 capture oligonucleotides are immobilized in one or more binding domains within a well of a multi-well plate, wherein each binding domain includes a capture oligonucleotide having a sequence that is different than the sequences of the capture oligonucleotides in the other binding domains within the well. In one aspect, the kit includes a support surface having at least 1, 2, 3, 4, 5, 6, 7, 8, 9, 10, 15, 20, or 25 distinct capture oligonucleotides immobilized in at least 1, 2, 3, 4, 5, 6, 7, 8, 9, 10, 15, 20, or 25 unique binding domains. In one aspect, the kit includes a multi-well plate having at least 1, 2, 3, 4, 5, 6, 7, 8, 9, 10, 15, 20, or 25 distinct capture oligonucleotides immobilized in at least 1, 2, 3, 4, 5, 6, 7, 8, 9, 10, 15, 20, or 25 unique binding domains in one or more wells. In one aspect, the kit includes one or more multi-well plates in which each well includes up to 10 capture oligonucleotides immobilized in an array. In one aspect, the multi-well plate can be configured to create between 1 and 10 detection assays within each well of the multi-well plate.

In one aspect, the kit includes a standard format multi-well plate, which are known in the art and can include, but are not limited to, 24, 96, and 384 well plates. In one aspect, the kit includes one or more 96 well plates. In one aspect, the kit includes one multi-well plate. In another aspect, the kit includes 10 multi-well plates. In another aspect, the kit includes between 10 and 100 multi-well plates.

In one aspect, a kit is provided for conducting a luminescence assay, for example, an electrochemiluminescence assay to identify, detect or quantify one or more target nucleotide sequences in a sample. In one aspect, the kit includes one or more assay components useful in carrying out an electrochemiluminescence assay.

In one aspect, the kit includes hybridization buffer that can be used to provide the appropriate conditions (e.g., stringent conditions) for hybridization of oligonucleotide tags to their corresponding complementary capture oligonucleotides sequences. In one aspect, the hybridization buffer includes a nucleic acid denaturant such as formamide. In one aspect, the hybridization buffer is provided as two separate components that can be combined to form the hybridization buffer.

In one aspect, the kit includes a container of wash solution for removing free (i.e., not immobilized) capture molecule from the support surface after printing. In one aspect, the wash solution is an aqueous solution. In one aspect, the wash solution includes a thiol-containing compound. In one aspect, the thiol-containing compound is water-soluble and has a molecular weight less than about 200 g/mol, 175 g/mol, 150 g/mol, or 125 g/mol. In one aspect, the thiol-containing compound is selected from cysteine, cysteamine, dithiothreitol, 3-mercaptoproprionate, 3-mercapto-1-propanesulfonic acid and combinations thereof. In one aspect, the thiol-containing compound includes cysteine. In one aspect, the thiol-containing compound includes a zwitterion.

In one aspect, the water-soluble thiol-containing compound in the wash solution competes with free capture oligonucleotide to prevent wash-over. Wash-over refers to a redepositing of capture molecules to a neighboring binding domain, for example, when a loosely bound capture molecule is released from the surface to into a solution, for example, a wash buffer, assay diluents, or sample, and migrates to one or more neighboring binding domains. To reduce wash-over, loosely bound capture molecule should be removed and redeposition should be prevented. Wash-over can increases apparent cross-reactivity between different analytes even if there is no true cross-reactivity.

While not wishing to be bound by theory, it is believed that mechanism of action of the wash solution is as follows: the wash solution brings loosely bound capture oligonucleotides into solution, from which they can potentially be re-deposited to the surface either via SH-covalent binding or other mechanisms. If a capture oligonucleotide is re-deposited on a binding domain with capture oligonucleotides having a different nucleotide sequence, it is considered a contaminating capture molecule. The presence of contaminating capture molecules can interfere with the assay results. In one aspect, the wash solution includes a water-soluble thiol containing compound, for example, cysteine, at great molar excess over the capture oligonucleotides (at least 10,000×), which allows the thiol-group of the thiol containing compound to bind and outcompete the loose capture oligonucleotides for binding to available sites on the surface. Triton X-100 (0.1%) inactivates surface reactivity with the SH-groups; and the Tris molecules reduce in-solution binding, possibly due to the presence of amine group that have the potential to bind to the surface. In one aspect, the binding domains of an array prepare by the methods described herein include less than about 0.1%, 0.09%, 0.08%, 0.07%, 0.06%, 0.05%, 0.04%, 0.03%, 0.02%, or 0.01% contaminating capture molecules.

In one aspect, the wash solution includes a thiol-containing compound, a pH buffering component, a surfactant, or combinations thereof and has a pH between about 7 and about 9.

In one aspect, the wash solution includes between about 5 mM and about 750 mM, between about 10 mM and about 500 mM, about 25 mM and about 75 mM, or about 50 mM cysteine. In one aspect, the surfactant is a non-ionic surfactant, for example, Triton X-100. In one aspect, the wash includes between about 10 mM and about 30 mM, or about 15 mM and about 25 mM, or about 20 mM of a buffer such as Tris. In one aspect, the wash includes between about 0.05% and about 0.5%, or between about 0.05% and 0.2%, or about 0.1% of a surfactant such as Triton X-100. In one aspect, the wash solution has a pH between about 7.5 and about 8.5, or about 8.0. In one aspect, the wash buffer includes between about 15mM and about 25 mM Tris, about pH 8.0, between about 0.05% and about 0.15% triton X-100 and between about 25 mM and 75 mM cysteine. In a more particular aspect, the wash includes about 20 mM Tris, about pH 8.0, about 0.1% triton X-100, and about 50 mM cysteine.

In one aspect, one or more components of the wash solution are provided in the kit in dry form. In one aspect, a liquid diluent is provided in the kit for reconstituting one or more components of the wash solution.

In one aspect, the kit includes one or more containers that include a label. In one aspect, the label is selected from a radioactive, fluorescent, chemiluminescent, electrochemiluminescent, light absorbing, light scattering, electrochemical, magnetic and an enzymatic label. In one aspect, the label includes an electrochemiluminescent label. In one aspect, the label includes an organometallic complex that includes a transition metal. In one aspect, the transition metal includes ruthenium. In one aspect, the label is a MSD SULFO-TAG™ label.

In one aspect, the label includes a primary binding reagent that is a binding partner of a secondary binding reagent. In one aspect, the secondary binding reagent includes biotin, streptavidin, avidin, or an antibody. In one aspect, the secondary binding reagent includes avidin, streptavidin or an antibody. In one aspect, the label includes a hapten selected from biotin, fluorescein and digoxigenin. In one aspect, the label is a primary binding agent that includes a first oligonucleotide sequence and the secondary binding reagent includes a second oligonucleotide sequence that is complementary to the first oligonucleotide sequence of the primary binding agent.

In one aspect, the kit includes one or more containers that include an electrochemiluminescent label. In a more particular aspect, the kit includes one or more containers containing Ru-containing or Os-containing organometallic compounds such as tris-bipyridyl-ruthenium (RuBpy). In one aspect, the label includes an organometallic complex that includes a transition metal. In one aspect, the transition metal includes ruthenium. In one aspect, the label includes the MSD SULFO-TAG™ label (MesoScale, Rockville, MD). In another aspect, the kit includes one or more containers containing luminol or other related compounds.

In one aspect, the kit includes one or more containers with one or more electrochemiluminescent co-reactants. In one aspect, one or more electrochemiluminescent co-reactants are covalently or non-covalently immobilized on the support surface. In one aspect, one or more electrochemiluminescent co-reactants are immobilized on one or more working electrodes of the support surface.

In one aspect, the label included in the kit includes a primary binding reagent and a secondary binding reagent. In one aspect, the secondary binding reagent includes biotin, streptavidin, avidin or an antibody.

In one aspect, the kit is adapted for multiple assays. In one aspect, the kit is contained in a resealable bag or container. In one aspect, the bag or container is substantially impermeable to water. In one aspect, the bag is a foil, for example, an aluminized foil. In one aspect, the kit and reagents are stored in a dry state and the kits may include desiccant materials to maintain the assay reagents in a dry state.

In one aspect, the kit includes a support surface that includes one or more immobilized capture oligonucleotides packaged in a desiccated package. In one aspect, the kit includes a support surface that was washed with a thiol-containing wash solution before it was is packaged in the desiccated package. In one aspect, the kit includes a support surface that includes one or more immobilized capture oligonucleotides, wherein the support surface was not washed with a thiol-containing wash solution before it was package in a desiccated package.

In one aspect, the kit includes one or more of the following assay components: one or more non-cross-reactive capture oligonucleotides; and one or more buffers, for example, a wash buffer, a hybridization buffer, a binding buffer, or a read buffer. In one aspect, the hybridization buffer includes a nucleic acid denaturant. In one aspect, the nucleic acid denaturant includes formamide. In one aspect, the hybridization buffer is provided as two separate components that can be combined to form the hybridization buffer. In one aspect, the binding buffer includes a surfactant. In one aspect the read buffer includes an electrochemiluminescent read buffer.

In one aspect, the kit includes one or more assay components such as a label. In one aspect, the label is a luminescent label such as an electrochemiluminescent label. In one aspect, the kit includes at least one electrochemiluminescence co-reactant. In one aspect, the electrochemiluminescent co-reactant includes a tertiary amine, tripropylamine, or N-butyldiethanolamine.

In one aspect, the label includes a primary binding reagent that is a binding pair of a secondary binding reagent. In one aspect, the kit includes the secondary binding reagent. In one aspect, the kit includes one or more assay components in dry form in one or more plate wells. In one aspect, the kit includes a unique kit identifier.

In one aspect, the kit includes one or more other assay components. In one aspect, the kit includes one or more assay including, but not limited to, a diluent, blocking agents, stabilizing agents, detergents, salts, pH buffers, and preservatives. In one aspect, the kit includes containers of one or more such components. In another aspect, one or more reagents are included on the assay support surface provided with the kit.

In one aspect, the kit includes a binding buffer that can be used to provide the appropriate conditions for binding one or more probes to one or more target nucleotide sequences. In one aspect, the binding buffer includes a surfactant.

In one aspect, the kit includes a read buffer that can be used to provide the appropriate conditions for detecting the presence of the label. In one aspect, the kit includes an electrochemiluminescence read buffer that includes one or more electrochemiluminescence co-reactants, including, for example, a tertiary amine, tripropylamine, and N-butyldiethanolamine. In one aspect, the kit includes instructions for use or a unique kit identifier.

In one aspect, the kit includes one or more assay components for detecting a single nucleotide polymorphism in a target nucleotide sequence. In one aspect, the kit includes one or more of the following components: a labeled oligonucleotide probe including a sequence complementary to a target sequence in a nucleic acid of interest; one or more blocking probes; one or more nucleoside triphosphates; one or more labeled nucleoside triphosphates; labeled dideoxy nucleoside triphosphate; a ligase, or a polymerase.

In one aspect, the kit includes one or more, or a plurality of labeled oligonucleotide probes having a first sequence complementary to a target sequence in a nucleic acid of interest and an oligonucleotide tag complementary to a capture oligonucleotide.

In one aspect, the kit includes one or more assay components for identifying, detection or quantifying a target nucleotide sequence using an oligonucleotide ligation assay, including, for example, ligase buffer or DNA ligase.

In one aspect, the kit includes one or more assay components for detecting, identifying or quantifying one or more target nucleotide sequences in a sample, wherein one or more target nucleotide sequences include a polymorphic nucleotide. In one aspect, the kit includes at least one pair of oligonucleotide probes. In one aspect, the kit includes a plurality of pairs of oligonucleotide probes for a plurality of target nucleotide sequence. In one aspect, the pair of oligonucleotide probes includes a targeting probe and a detecting probe. In one aspect, the targeting probe includes a single stranded oligonucleotide tag that is complementary to at least a portion of a capture oligonucleotide immobilized on the support surface and a first nucleic acid sequence that is complementary to a first region of the target nucleotide sequence in the sample. In one aspect, the detecting probe includes a label and a second nucleic acid sequence that is complementary to a second region of the target nucleotide sequence that is adjacent to the first region to which the first nucleic acid sequence of the targeting probe sequence is complementary, wherein the targeting or detecting probe includes a terminal 3′ or 5′ nucleotide situated over the polymorphic nucleotide of the target nucleotide sequence. In one aspect, the label is attached to a 3′ end of the detecting probe.

In one aspect, the targeting probe has a terminal 3′ nucleotide complementary to a region of the target nucleotide sequence adjacent to the region to which the 5′ terminal nucleotide of the detecting probe is complementary. In one aspect, the terminal 5′ nucleotide of the detecting probe is complementary to the polymorphic nucleotide of the target nucleotide sequence.

In one aspect, the kit includes first and second detecting probes that bind the target nucleotide sequence, wherein the first and second detecting probes differ only in the terminal 5′ nucleotide. In one aspect, the first detecting probe is complementary to a wild type sequence and the second detecting probe is complementary to a mutant sequence.

In one aspect, the kit includes a ligase. In one aspect, the kit includes one or more nucleoside triphosphates.

In one aspect, the kit or method includes one or more blocking probes. In one aspect, one or more blocking probes are used to increase assay sensitivity, for example, for the detection of rare or low-allele fractions of cancer mutations. In one aspect, blocking probes are used to reduce background signals in an OLA assay by preventing template molecules from bridging non-ligated probes into complexes that can hybridize with the capture oligonucleotides and generate false signals from unligated probes. In one aspect, the blocking probe includes a single stranded nucleotide sequence that is complementary to a target nucleotide sequence and straddles a probe ligation site but does not include a tag or label. In one aspect, the blocking probe is largely colinear with the probe sequences. In one aspect, a pair of blocking probes is used that includes a first blocking probe having a sequence identical to the wild type or variant targeting probe used in an OLA assay and a second blocking probe having a sequence that is identical to the detecting probe, but does not include a 5′ phosphate or a 3′ label.

In one aspect, the blocking probe includes at least about 20, 25, 30, 35, 40, 45 or 50 and up to about 50, 75, 100, 150, or 200, or between about 20 and about 200, or between about 50 and about 100 nucleotides. In one aspect, a pair of blocking probes is included in the ligation reaction mixture, in which the first blocking probe has a sequence identical to the connection probe, but without the oligonucleotide tag; and the second blocking probe has a sequence identical to the detecting probe, but without the label. In one aspect, up to 2, 3, 4 or 5 additional nucleotides can be added to the 5′- and 3′-end of the blocking probe that are complementary to the target nucleotide sequence adjacent to the probe sequences. In one aspect, the kit includes at least one pair of blocking probes for each pair of oligonucleotide probes.

In one aspect, the kit includes one or more components for use in a primer extension assay. In one aspect, the kit includes one or more targeting probes for use in a primer extension assay. In one aspect, the kit includes a plurality of probes including targeting nucleic acid sequences that are complementary to a plurality of target nucleotide sequences in the sample. In one aspect, the kit includes other assay components for a primer extension assay including, for example, a polymerase, one or more nucleoside triphosphates or one or more dideoxynucleotide triphosphates (ddNTPs). In one aspect, the kit includes one or more labeled or unlabeled nucleoside triphosphates. In one aspect, the kit includes labeled or unlabeled dideoxy nucleoside triphosphate.

In one aspect, the targeting probe includes a single stranded oligonucleotide tag that is complementary to at least a portion of a capture oligonucleotide immobilized on the support surface; a targeting nucleic acid sequence that is complementary to a target nucleotide sequence in the sample; and a label. In one aspect, the oligonucleotide tag is attached to a 5′ end of the targeting probe and the targeting nucleic acid sequence has a 3′ end that is complementary to a nucleotide adjacent to a polymorphic nucleotide in one or more target nucleotide sequences in the sample. In one aspect, the oligonucleotide tag is attached to a 5′ end of the targeting probe and the targeting nucleic acid sequence includes a terminal 3′ nucleotide complementary to a polymorphic nucleotide of in one or more target nucleotide sequences in the sample.

In one aspect, the kit includes one or more target specific probes that include an oligonucleotide tag that binds to a capture oligonucleotide on the support surface provided with the kit and a binding partner specific to a target analyte. In one aspect, the kit includes one or more target specific probe having an oligonucleotide tag and a nucleic acid sequence that hybridizes to a nucleic acid sequence in one or more target analytes. In one aspect, the end user generates one or more target specific probes for one or more target analytes of interest.

In one aspect, the kit includes labeled nucleoside triphosphate. In one aspect, the kit includes labeled nucleoside triphosphate and a secondary binding reagent. In one aspect, the labeled nucleoside triphosphate includes a primary binding reagent that is a binding partner of a secondary binding reagent. In one aspect, the secondary binding reagent includes avidin, streptavidin or an antibody and the labeled nucleoside triphosphate includes a biotin or hapten label. In one aspect, the labeled nucleoside triphosphate includes a radioactive, fluorescent, chemiluminescent, electrochemiluminescent, light absorbing, light scattering, electrochemical, magnetic or enzymatic label. In one aspect, the kit includes nucleoside triphosphate labeled with an electrochemiluminescent label. In one aspect, the kit includes labeled dideoxy nucleotide triphosphate complementary to the polymorphic nucleotide of the target nucleotide sequence.

In one aspect, the kit includes a support surface, such as a multi-well plate, for example, a 96 well plate, wherein each well of the multi-well plate includes one or more capture oligonucleotides immobilized in one or more binding domains. In one aspect, each well of the multi-well plate includes between 1 and 10 binding domains, wherein a unique capture oligonucleotide is immobilized in each binding domain in a well. In one aspect, the kit also includes one or more of the following reaction components: wash buffer, hybridization buffer, label, diluent and read buffer. In one aspect, the wash buffer includes a thiol-containing compound. In one aspect, the wash buffer is an aqueous solution. In one aspect, the thiol-containing compound is water-soluble and has a molecular weight less than about 200 g/mol, 175 g/mol, 150 g/mol, or 125 g/mol. In one aspect, the thiol-containing compound is selected from cysteine, cysteamine, dithiothreitol, 3-mercaptoproprionate, 3-mercapto-1-propanesulfonic acid and combinations thereof. In one aspect, the thiol-containing compound includes cysteine. In one aspect, the label includes an electrochemiluminescent label. In one aspect, the label includes a secondary binding partner. In one aspect, the label includes MSD Sulfo-Tag labeled streptavidin.

### Q. Databases

Various databases are available that provide information about the genetic association of diseases and disorders and provide information and sequences that can be used in connection with the methods and kits described herein, including, but not limited to the following:

### Genetic Association Database

Database of genetic association data from complex diseases and disorders. Database is “frozen” as of Sep. 1, 2014. However, all data as of Aug. 18, 2014 is available for download in text or SQL format. geneticassociationdb.nih.gov

### ClinVar

NCBI database, includes filters to display results by pathogenicity, type of mutation, etc. ncbi.nlm.nih.gov/clinvar?term=human%5Borgn%5D

### New England Biolabs

Provides a list of common genes of interest. neb.com/tools-and-resources/usage-guidelines/genetic-markers

### Genome in a Bottle (GIAB) (The Joint Initiative for Metrology in Biology)

Public-private-academic consortium hosted by NIST to develop the technical infrastructure to enable translation of the whole human genome sequencing to clinical practice. Provides genomes for highly characterized reference materials jimb.stanford.edu/giab-resources/

### ENSEMBL Genome Browser

Ensembl is a genome browser for vertebrate genomes that creates, integrates and distributes reference datasets and analysis tools for genomics research. ensembl.org/index.html

### COSMIC Genome Browser

Provides a catalogue of somatic mutations found in cancer cancer.sanger.ac.uk/cosmic/browse/genome

### NCI Genomic Data Commons

Provides the cancer research community with a unified data repository that enables data sharing across cancer genomic studies in support of precision medicine. portal.gdc.cancer.gov

### NCBI Resources

dbSNP and dbVAR covering SNPs and other variants (insertions, deletions, translocations etc) ncbi.nlm.nih.gov/snp ncbi.nlm.nih.gov/dbvar

### Database of Genomic Variants Archive

A repository that provides archiving, accessioning and distribution of publicly available genomic structural variants, in all species. ebi.ac.uk/dgva

### IGSR: The International Genome Sample Resource

Repository for the 1000 genome project internationalgenome.org/data

### InSiGHT Variant Databases

InSiGHT houses and curates the most comprehensive database of DNA variants re-sequenced in the genes that contribute to gastrointestinal cancer. insight-group.org/variants/databases

### The UCSC Genome Browser

genome.ucsc.edu

### R. Incorporation by Reference

All references cited herein, including patents, patent applications, papers, text books and the like, and the references cited therein, to the extent that they are not already, are hereby incorporated herein by reference in their entirety for all purposes.

S. Capture Oligonucleotides

TABLE 1

Capture Oligonucleotide set 1: 36-mer non-cross-reactive capture oligonucleotides generated using base oligonucleotide #1

| Run | Base | Transform | Parent SEQ ID NO | SEQ ID NO | Sequence |
|---|---|---|---|---|---|
| 1 | 1 | None | 1 | 1 | ACCGATCATGTCTGGGTTACCAGTTAGTCGTGTCTC |
| 1 | 1 | None | 2 | 2 | TCGTCTTGAACCAATGACCAAATGCAAGCCCTCCAT |
| 1 | 1 | None | 3 | 3 | ATACGAGGGCACAGGAGCTATTAGTGTAGCGAAAGG |
| 1 | 1 | None | 4 | 4 | TCACCACCTGATTTCTGTTGCCACCGCATCAGTTTA |
| 1 | 1 | None | 5 | 5 | TTTCCATACCTGCAGCGGCATCTATTCATGACATGT |
| 1 | 1 | None | 6 | 6 | CCATTAAGCTCACCCACAGGGAGTTGGAGTCTAAAC |
| 1 | 1 | None | 7 | 7 | GTTAGAAGGACCACAACGGACCAGAGAGTGCATATA |
| 1 | 1 | None | 8 | 8 | AATTCTCAGGCTAGTCGACGGATTTACCGTCACTCG |
| 1 | 1 | None | 9 | 9 | CCTACAAACCTTTAGCAGTCCTCTGTTGGTCTCTGC |
| 1 | 1 | None | 10 | 10 | AAGGTCTCCAGATTCAATGGTACGACCATCCGACTC |
| 1 | 1 | None | 11 | 11 | AACTGTCTGTTCTCTGTGAGGAATTCTCCTTCCGAG |
| 1 | 1 | None | 12 | 12 | TCACGTAACCATTGTCGTTTAATGTCTCCCAGCGTT |
| 1 | 1 | None | 13 | 13 | CTATGCCAGATGATGCTATCCCGACGATGAACGGTT |
| 1 | 1 | None | 14 | 14 | TCCCTACGTCACAACTACCTATTCGAGTGTGGTCTT |
| 1 | 1 | None | 15 | 15 | GTGCTATCTAGCTAACCAAGCGGGCTCTTTATAGGC |
| 1 | 1 | None | 16 | 16 | ATACTAAGGGTTACTGCCATAGTGCCGACGCGTAGA |
| 1 | 1 | None | 17 | 17 | AGGTGTTTCTTTCTAGACACCAAGTATCCACAGGCA |
| 1 | 1 | None | 18 | 18 | CGAGACGGAGCAACGTTTGCTTTCAGTTAATCGGAG |
| 1 | 1 | None | 19 | 19 | GACGGAATCTTACATAAAGTGTTTGGAGATGGTAGG |
| 1 | 1 | None | 20 | 20 | CAGTACCGTGATTTAAGTCGGGTAGACGATGCGGAT |
| 1 | 1 | None | 21 | 21 | GTCTCTACGTTATACCGGATTTGGGTATTCTCTGAG |
| 1 | 1 | None | 22 | 22 | CTTTCTTTGAGACTGCGGGAAAGCGGTTCGGTAACT |
| 1 | 1 | None | 23 | 23 | GTACTTACACGGCTTGGCTCAGTGCCCGTTTCATAT |
| 1 | 1 | None | 24 | 24 | CTCGGTGTTCTGTAGGTAAATAACGAGTAATCGCAC |
| 1 | 1 | None | 25 | 25 | GTAACATCCCAAGCGAACCTGGCCTTTAGTACCCAA |
| 1 | 1 | None | 26 | 26 | GGGTAGTGCTGCAACAGTCGCGAATTATAAATACGG |
| 1 | 1 | None | 27 | 27 | AGTCAGTCATTTATCACACACATACACAGTGAGCTC |
| 1 | 1 | None | 28 | 28 | TTCAAAGATGCTCATCACCCTTCGCATCTCGGACCA |
| 1 | 1 | None | 29 | 29 | ATAGTCTCGAGTGCGCCTGTCCCTCTTACAGTTTCT |
| 1 | 1 | None | 30 | 30 | TCCTACGCGATCTACCTGTATTACTTTACTGGCAAC |
| 1 | 1 | None | 31 | 31 | TTCTGCGGAAGCTGATCCGTCACACAATCCTTCTTG |
| 1 | 1 | None | 32 | 32 | TGGTTCCGCCGCTTGAATACAACCAATACTTATCGG |
| 1 | 1 | None | 33 | 33 | GTGAGCGTTTACGAGCAGTACGCTTCAACTCAATTC |
| 1 | 1 | None | 34 | 34 | CAGGGTGTTATTGTTGGAACGCCAAATCGTCTGAAC |
| 1 | 1 | None | 35 | 35 | CGCTCAAATGAACTTTCAACATCGAATCCTTGCTGG |
| 1 | 1 | None | 36 | 36 | TGGTATTGGGCAGATGCTTCTTAGGAATTGTGCAGT |
| 1 | 1 | None | 37 | 37 | ATCTTGCCATGCACGAAATTTACGATTAGGTCAGCG |
| 1 | 1 | None | 38 | 38 | TATACTACTTAAACAGGAACCCGCTCTCCGCAGGAC |
| 1 | 1 | None | 39 | 39 | GTAGATCCCTACATTCAGAAATGCGTCTGTTGACGG |
| 1 | 1 | None | 40 | 40 | GTGTGCTAAGTGCGCGAATATAATAGCAAGTAGTTG |
| 1 | 1 | None | 41 | 41 | CGCAAGCAGATAATGAGTTAGTTAGGCACACATCAT |
| 1 | 1 | None | 42 | 42 | AGTATCACGCTCCTAGGCTTGTAACAGATTGGCTAG |
| 1 | 1 | None | 43 | 43 | CCATACAATTCGTCAATCTATGTGACACTGTCCACC |
| 1 | 1 | None | 44 | 44 | ACATGACGGGTTGCTCAACATAAGACGTTACTCAGC |
| 1 | 1 | None | 45 | 45 | CGGCGGTTAATAAACGTAACGACATGAGTGTCCCTG |
| 1 | 1 | None | 46 | 46 | TTTCAAGTATTTCCGACATTCCTGCCTAGTTCCGCG |
| 1 | 1 | None | 47 | 47 | TTCGTAACTAGACTAGCGTACGTCGCTATAGGTCTT |
| 1 | 1 | None | 48 | 48 | AGTGGAATTCGGTGCCGCGTATAAATCAACTGACTG |
| 1 | 1 | None | 49 | 49 | TTTGCCTCATCCTTACACAAGACACCTTCTCCTACA |
| 1 | 1 | None | 50 | 50 | GATATCAACAACCCACCCTGCCTTATCTTCGTGTAT |
| 1 | 1 | None | 51 | 51 | CCAGATATAGTTAAATTAGCTGCGCGTGTACATACG |
| 1 | 1 | None | 52 | 52 | TGAATATTACCCGTCCCGCGACCTTCAATGAGTCGT |
| 1 | 1 | None | 53 | 53 | TCATCAATTGGGACATGTTATTCCTCCGTAAGCTTG |
| 1 | 1 | None | 54 | 54 | TTATAGAACAAAGTCAAGGGCCGTGCGTAATTCGGG |
| 1 | 1 | None | 55 | 55 | GCCTCAGTAGGCCCTAAGTTAACATCAGCTATGTAC |
| 1 | 1 | None | 56 | 56 | AGCAGGCCAATGGAGTGGTAATTCATCTTGGCCTCA |
| 1 | 1 | None | 57 | 57 | TAAGTGTCGGCTCATATCTAAAGCAGACGGACATAA |
| 1 | 1 | None | 58 | 58 | CATTGATATGATGATCCTCGAGCCGGTCGAACTTAC |
| 1 | 1 | None | 59 | 59 | GGAAGTACTGGCGTCTTTCGACGCTTATCAAACTAC |
| 1 | 1 | None | 60 | 60 | AATATACGAGAAACCCATAAGATAGCGCGCATTCGT |
| 1 | 1 | None | 61 | 61 | AACAGCTACTACCTGGGTCTTAATGCGGAGGTTGGA |
| 1 | 1 | None | 62 | 62 | CCCACTGATCTAACATGAGGCCGGTGGGATTTGAAA |
| 1 | 1 | None | 63 | 63 | CCGACCTCGTACTAACCGCTGCCCTGATAAAGAATT |
| 1 | 1 | None | 64 | 64 | CGTTCGTGACAAAGATCCGGGCCTCTCTATCATAGT |

TABLE 2

Capture Oligonucleotide set 2: 36-mer non-cross-reactive capture oligonucleotides generated using base oligonucleotide #2

| Run | Base | Transform | Parent SEQ ID NO | SEQ ID NO | Sequence |
|---|---|---|---|---|---|
| 2 | 2 | None | 65 | 65 | GTCGTCGTTCAGAGTACGTATCAAAGGAATGATCAA |
| 2 | 2 | None | 66 | 66 | AACAGCACGGCCTCCAGATCCTCTGTCTTTGCATAA |
| 2 | 2 | None | 67 | 67 | TTACTGAAACGCTGAGTCTTTCCGCTTAGCTACTGG |
| 2 | 2 | None | 68 | 68 | ATTCTTCGAACGAGCCATTCAATTGGTATACGGAGT |
| 2 | 2 | None | 69 | 69 | TTAGAGAGCGCGTGCACTATTTGTAGGTAGGTATGC |
| 2 | 2 | None | 70 | 70 | ATCTGGTCTTGAGATTAAATAGCTTTGCCGGTTGTG |
| 2 | 2 | None | 71 | 71 | TAGCTTAATCTGTTCGCACAAAGTACTGCTCGTCCC |
| 2 | 2 | None | 72 | 72 | CCGCAATTTCTCAATTACGACTAAAGCTCTCGCGGC |
| 2 | 2 | None | 73 | 73 | GACCGCACTATATTTCAGCTCTCATTGGGCATTGCA |
| 2 | 2 | None | 74 | 74 | AGCGGCTTGTTTAGACTCTATTCCTGAGGACCTGAC |
| 2 | 2 | None | 75 | 75 | CATACCAACGACAGTCCAATCACTTTCCTTCACTCC |
| 2 | 2 | None | 76 | 76 | TATATCGAAATCACCGCAACGACTCGCTTTCTCATT |
| 2 | 2 | None | 77 | 77 | ATGTTTAAAGAAATCCCGGACGGCTATGTCAAGCGG |
| 2 | 2 | None | 78 | 78 | CAGCTATACTATCATGTGTTGATCGGAGACCGCTGA |
| 2 | 2 | None | 79 | 79 | AATAACAGCGTGGTATATCATCCGACGTGTCTATCT |
| 2 | 2 | None | 80 | 80 | GTATGTAGAAGGTCAACTGCAGCGAGCGAATTCCTT |
| 2 | 2 | None | 81 | 81 | TCGCTATTTGACTTGTATAGGTCCCTCCACTAGGTA |
| 2 | 2 | None | 82 | 82 | AAAGTGGGTTGGTACCCGATTCTTATCAAACTCTAC |
| 2 | 2 | None | 83 | 83 | GACGCATTAGTTGTCTAGAACCATCATCAACCTGTC |
| 2 | 2 | None | 84 | 84 | ACTATTAACACCATCAGGGTCAATGCCATGGAAGGT |
| 2 | 2 | None | 85 | 85 | TACGAGTTAGGTCCATGTGAACGCATAGGCTGCGAA |
| 2 | 2 | None | 86 | 86 | TAGTCAAGGTCTTTCACCTGTTGCCGCTGTATATAT |
| 2 | 2 | None | 87 | 87 | ATATTATTAGCACGCCCGAGTATTGCTTAGGCCGAC |
| 2 | 2 | None | 88 | 88 | ATGAATCTACGTGGCGTTGTGTCGGGTATCGTCGAT |
| 2 | 2 | None | 89 | 89 | AGTTACTCACCGTGGACGAATAAACATTGCTAGCCC |
| 2 | 2 | None | 90 | 90 | CCCTCGCGAAACTTGAAGCTACAGACATGTGCATGA |
| 2 | 2 | None | 91 | 91 | GACTGATTCCTCTACTTACTGTCTGGATGGACAGGT |
| 2 | 2 | None | 92 | 92 | ACAATCAGGCAGGATGACACCAATCTGGCTAGACTC |
| 2 | 2 | None | 93 | 93 | TGGAGCGTAAGCCTTGGAGCCTTGATCTAGAATGAA |
| 2 | 2 | None | 94 | 94 | CGAAGCGTCTTAACCTTAGAACTTTCCAGTGAGTGG |
| 2 | 2 | None | 95 | 95 | CGAACATTCAGGGTTCTGGTTCGTCAGTCGCCTAAA |
| 2 | 2 | None | 96 | 96 | TCCTCAATCGCTCTACATCCGAGGAGCAAGATACAA |
| 2 | 2 | None | 97 | 97 | TGTGTTGGGACGGTAATGAGGACACAATCGATCAGT |
| 2 | 2 | None | 98 | 98 | AGCTTACTCAAACAAGTTAGCACTGAAGGCTACACA |
| 2 | 2 | None | 99 | 99 | TCGAATTGCAGCACCGACCTTGTGAGTCCTAAACAT |
| 2 | 2 | None | 100 | 100 | AGCGAAGTGAGAGAGAATGGTGATCCGTGTGATTAT |
| 2 | 2 | None | 101 | 101 | TGGACCGGAAGGGTTAATCGTATGCGGCATGAACAA |
| 2 | 2 | None | 102 | 102 | AATAGGGACTCTAACTCAATCTCGTGACAGCATACA |
| 2 | 2 | None | 103 | 103 | AGGCTCGTCAAATGGTCAAACCTTCACAAACAACTC |
| 2 | 2 | None | 104 | 104 | GGACCGTTCTACTCGACGAACTTACACTTGGTCGTA |
| 2 | 2 | None | 105 | 105 | TGCGACAGTTGCTACATGTCCTCTTACCACCCTTCA |
| 2 | 2 | None | 106 | 106 | GCCGTGAATCGTGCTTTGGATGCTCAATATACACTA |
| 2 | 2 | None | 107 | 107 | CTATCTGCTACTCAGAGAAACGAGGTTCAGGATCTC |
| 2 | 2 | None | 108 | 108 | GATCCTGGGATTATTGATGTGGCACCCAAACGCAAG |
| 2 | 2 | None | 109 | 109 | GCGGAACCACAGCTTTCTTAGGTTGCATCAATTTAG |
| 2 | 2 | None | 110 | 110 | ATCTGTGCGGTAGATGCACGTCATTAGTCTACTATA |
| 2 | 2 | None | 111 | 111 | CCGACGTTATTCGATTCGGGAAACAGACTGTGCTTC |
| 2 | 2 | None | 112 | 112 | TCTGGCGCTGGGTAGTAACGTAACACAGTTTAATTA |
| 2 | 2 | None | 113 | 113 | AGTGGGCGCAGAACAACCGCAGTTAAGATAACACTA |
| 2 | 2 | None | 114 | 114 | CGTACGTAGGGACACCGACATGAGATATAACATAGA |
| 2 | 2 | None | 115 | 115 | CATTTCGCCGTCTTCGTAACAACAACGGCGTTTCGT |
| 2 | 2 | None | 116 | 116 | ACGAGTGACGGAGTGACTGGGTTTGGAATTATGCTT |
| 2 | 2 | None | 117 | 117 | GTACTTCAGCGCGGTGCGTGTAGCATGAGAATTATC |
| 2 | 2 | None | 118 | 118 | TGGCTCTTGAACACGTAACGAACTATCAATGCGGTT |
| 2 | 2 | None | 119 | 119 | TTAAACAATAAGATCCCAGAACGGAGCCTGGCCCAA |
| 2 | 2 | None | 120 | 120 | ATGAACACTCTCCATCTTGCACTAAGTCAGGAAGCA |
| 2 | 2 | None | 121 | 121 | CCCTCAGATTCTGTGATTCCACTTTATAGGACACGG |
| 2 | 2 | None | 122 | 122 | GACATCATATACGTGAACAGCAGGAGAACCAATACG |

TABLE 3

Capture Oligonucleotide set 3: 36-mer non-cross-reactive capture oligonucleotides generated using base oligonucleotide #3

| Run | Base | Transform | Parent SEQ ID NO | SEQ ID NO | Sequence |
|---|---|---|---|---|---|
| 3 | 3 | None | 123 | 123 | GCTACTGTGGAGAGGGTTTGTGAATCTAGGAGCACA |
| 3 | 3 | None | 124 | 124 | ACAGCAATGCTAGGGAGCAATAAACATAACCATCCA |
| 3 | 3 | None | 125 | 125 | GAGACGTTCTCTTTCCATTTGGATCATTTCGGACCG |
| 3 | 3 | None | 126 | 126 | AACCAGAAGTTGTGGCCCATACTCGTTTACTGGGTG |
| 3 | 3 | None | 127 | 127 | GCGAATCGTAACTCCACCACAGAGTACGACGATTTG |
| 3 | 3 | None | 128 | 128 | CGGTCGTAGCCCTTATATTCGCTCAGTACGATTGAC |
| 3 | 3 | None | 129 | 129 | GGTTCCGCTTGCGACCGTTTAGATGTTTCAGAACAG |
| 3 | 3 | None | 130 | 130 | GGCTGTTCGCGTGATACGTCGTAAACCTAGATAGTC |
| 3 | 3 | None | 131 | 131 | AACCATGTCTAGTATTTGTCACGTCCTGTATGACCG |
| 3 | 3 | None | 132 | 132 | TGTACTTCGCCACACCTGTCCTTGTGGTTTGCCTAA |
| 3 | 3 | None | 133 | 133 | ACTAGGGTCCTTCAGAGCCGGTAGATGTATGGCATA |
| 3 | 3 | None | 134 | 134 | TTTACACTGGTATAGGGACGGTGTGTAGCCGAGCTA |
| 3 | 3 | None | 135 | 135 | GTGGGTTAAATATGATTTGGAGGACGAGACGCGCAT |
| 3 | 3 | None | 136 | 136 | AAAGTTCTGACGGCTATTCGCAGTTTCCACGGAACT |
| 3 | 3 | None | 137 | 137 | AGATTGCCTTCGTGTGCAGAATAGCGGCATCGTCTT |
| 3 | 3 | None | 138 | 138 | ATTCCAAGGCGATACGGGCTCGTCTTAACGGGAATT |
| 3 | 3 | None | 139 | 139 | TATAAGCCTCGCCTGACAGACGTTATTGTCTACACA |
| 3 | 3 | None | 140 | 140 | TGCTGGCTTAACGCCCATCTACACTTAGCTATAGAA |
| 3 | 3 | None | 141 | 141 | CGGGAGGTTATAAACCGCACATAGTAAATAGCTCAA |
| 3 | 3 | None | 142 | 142 | ATCAGTTCGCCTGTGAGCAGCAGCTAATACCTGTAA |
| 3 | 3 | None | 143 | 143 | AAAGGTATCAACTACGTATACCTGGGATGAACAGAC |
| 3 | 3 | None | 144 | 144 | CTCCAGGGATAGTTTCTATGAGTTTGAACAACGTCG |
| 3 | 3 | None | 145 | 145 | CTCTATTTGACGAACTGTCTGTAAGCACCCAAGGAT |
| 3 | 3 | None | 146 | 146 | GAGTTATATGAAGGAAAGTGTCTCGGCCGTACTTTC |
| 3 | 3 | None | 147 | 147 | GTCTGGTCGTGTACCCACAAATATAGGGCTGTCTTG |
| 3 | 3 | None | 148 | 148 | AACATCCAGATAGCGAAACCAGTCTTTACTTTGGCC |
| 3 | 3 | None | 149 | 149 | ACTAAAGCGCTCGATCCACCATTTCTTGAACTGCAA |
| 3 | 3 | None | 150 | 150 | GGACATGTAGTCTAACACTGGGCGTCATAGGATTGC |
| 3 | 3 | None | 151 | 151 | ATCTTCGAACTCGCTTCAACCTGGACTGTGCTGTTA |
| 3 | 3 | None | 152 | 152 | TCCCGTGCTCAATTGCGATTACTACAAAGAGTAGCC |
| 3 | 3 | None | 153 | 153 | TCAATTTCTCGCCGGAGTTTGCCACTGCTTCCTATG |
| 3 | 3 | None | 154 | 154 | ATCACTATACTATGGACGCATGGAGAGTGGGTATCC |
| 3 | 3 | None | 155 | 155 | CACGGTTTGATTAGATGCAATAGCGTTGGCTGAATG |
| 3 | 3 | None | 156 | 156 | CTACTCTCTGAATACATTATCCGAGTGGGCGAGGTT |
| 3 | 3 | None | 157 | 157 | CCGCTGGTAAGTTGATTGTGCAACCCGTAACCTTTA |
| 3 | 3 | None | 158 | 158 | AGGAATAAAGCGACATAAGAAGAGCATGCACTCTTG |
| 3 | 3 | None | 159 | 159 | CTGACTCCTAAGTGATGAGAACATATAGCCCACAGG |
| 3 | 3 | None | 160 | 160 | AATCGTTCGTTAGTGCTACGCCTTCACTTAAGCTAT |
| 3 | 3 | None | 161 | 161 | CAACCTGTATCGGAGACCATTTGTAATCACATCGCC |
| 3 | 3 | None | 162 | 162 | ATCAACGTTTGCAATAAGATTCAGCTGGAGTAGAGC |
| 3 | 3 | None | 163 | 163 | ACTATGCTCCGGTAATGGGTCATTAGATTCGAAGGA |
| 3 | 3 | None | 164 | 164 | CTACATCGACGAATGCTTTGTCCACTATTAACGTCG |
| 3 | 3 | None | 165 | 165 | TTATCGTGGTGTGATAACTGATTTGCTTTCGGGAGT |
| 3 | 3 | None | 166 | 166 | GGATCTACAGTGACTCTATCGGGTTGGGTAGTTCTT |
| 3 | 3 | None | 167 | 167 | ATTCCTGACCGGATGGCTGTAGGACATAGTTGTAAG |
| 3 | 3 | None | 168 | 168 | ATGCTGACGCTGAGGTACGCTAACAGGACAAATCCA |
| 3 | 3 | None | 169 | 169 | ACAATTAGCGGCCATATCTGTTAAGTCATTCCTCCG |
| 3 | 3 | None | 170 | 170 | TGCATAAAGAATCCTCGGAGTAGTTGGATCCTGATG |
| 3 | 3 | None | 171 | 171 | GGACAGGCCAGTTAAACATTGCGGGAAGCTTAACTA |
| 3 | 3 | None | 172 | 172 | TTTGCGCCCGGTGGTTAATCCCTAATAGATCTCACT |
| 3 | 3 | None | 173 | 173 | GGTTGGTGTCTGCAAATTGCTGGCGTTGGTAATCTG |
| 3 | 3 | None | 174 | 174 | ACGCTGTATCTCCGGCTGTCAATATGTGAATTCCGC |
| 3 | 3 | None | 175 | 175 | TCCACTTTAGTCTGCAGTCGGTGCTCTCTTACTCTA |
| 3 | 3 | None | 176 | 176 | AGTAATTAAGGCTTCCCATTGATCCGCCGAGCATTA |
| 3 | 3 | None | 177 | 177 | CAGAATATACCTTCGGTAGCACAGCAGACCTTAGGT |
| 3 | 3 | None | 178 | 178 | CCGAAACTGTTGATCATCGCGCTTTCAAACGGGTTA |
| 3 | 3 | None | 179 | 179 | ATGACTCGGCGATCTTGTCTGGGAGCTAGCAAATTC |
| 3 | 3 | None | 180 | 180 | GGGTCACTACGTTAAAGTGTTGGTATGGCCCTCTAA |
| 3 | 3 | None | 181 | 181 | TTCAACACCGTTATGGATCCGTGCCGAATCAGATCG |
| 3 | 3 | None | 182 | 182 | TCAGTCTGTATGGAGTATCGGCACTTCCACATCCTG |
| 3 | 3 | None | 183 | 183 | TGCGGGCAATAGTAGCTTGGATCTCGTGCAATTAGG |
| 3 | 3 | None | 184 | 184 | AATTCCGGTTTACCGTCGCTCACATTTCCTGGAGAC |
| 3 | 3 | None | 185 | 185 | AGTTGTGTTGTGCGAAATTAGGCGGATGCTACGGGA |
| 3 | 3 | None | 186 | 186 | ACGTTGCCTGGCTGAGTGTGTTAATGATGTCTCGAT |

TABLE 4

Capture Oligonucleotide set 4: 36-mer non-cross-reactive capture oligonucleotides complementary to the sequences generated using base oligonucleotide #1

| Run | Base | Transform | Parent SEQ ID NO | SEQ ID NO | Sequence |
|---|---|---|---|---|---|
| 4 | 1 | Complement | 1 | 187 | GAGACACGACTAACTGGTAACCCAGACATGATCGGT |
| 4 | 1 | Complement | 2 | 188 | ATGGAGGGCTTGCATTTGGTCATTGGTTCAAGACGA |
| 4 | 1 | Complement | 3 | 189 | CCTTTCGCTACACTAATAGCTCCTGTGCCCTCGTAT |
| 4 | 1 | Complement | 4 | 190 | TAAACTGATGCGGTGGCAACAGAAATCAGGTGGTGA |
| 4 | 1 | Complement | 5 | 191 | ACATGTCATGAATAGATGCCGCTGCAGGTATGGAAA |
| 4 | 1 | Complement | 6 | 192 | GTTTAGACTCCAACTCCCTGTGGGTGAGCTTAATGG |
| 4 | 1 | Complement | 7 | 193 | TATATGCACTCTCTGGTCCGTTGTGGTCCTTCTAAC |
| 4 | 1 | Complement | 8 | 194 | CGAGTGACGGTAAATCCGTCGACTAGCCTGAGAATT |
| 4 | 1 | Complement | 9 | 195 | GCAGAGACCAACAGAGGACTGCTAAAGGTTTGTAGG |
| 4 | 1 | Complement | 10 | 196 | GAGTCGGATGGTCGTACCATTGAATCTGGAGACCTT |
| 4 | 1 | Complement | 11 | 197 | CTCGGAAGGAGAATTCCTCACAGAGAACAGACAGTT |
| 4 | 1 | Complement | 12 | 198 | AACGCTGGGAGACATTAAACGACAATGGTTACGTGA |
| 4 | 1 | Complement | 13 | 199 | AACCGTTCATCGTCGGGATAGCATCATCTGGCATAG |
| 4 | 1 | Complement | 14 | 200 | AAGACCACACTCGAATAGGTAGTTGTGACGTAGGGA |
| 4 | 1 | Complement | 15 | 201 | GCCTATAAAGAGCCCGCTTGGTTAGCTAGATAGCAC |
| 4 | 1 | Complement | 16 | 202 | TCTACGCGTCGGCACTATGGCAGTAACCCTTAGTAT |
| 4 | 1 | Complement | 17 | 203 | TGCCTGTGGATACTTGGTGTCTAGAAAGAAACACCT |
| 4 | 1 | Complement | 18 | 204 | CTCCGATTAACTGAAAGCAAACGTTGCTCCGTCTCG |
| 4 | 1 | Complement | 19 | 205 | CCTACCATCTCCAAACACTTTATGTAAGATTCCGTC |
| 4 | 1 | Complement | 20 | 206 | ATCCGCATCGTCTACCCGACTTAAATCACGGTACTG |
| 4 | 1 | Complement | 21 | 207 | CTCAGAGAATACCCAAATCCGGTATAACGTAGAGAC |
| 4 | 1 | Complement | 22 | 208 | AGTTACCGAACCGCTTTCCCGCAGTCTCAAAGAAAG |
| 4 | 1 | Complement | 23 | 209 | ATATGAAACGGGCACTGAGCCAAGCCGTGTAAGTAC |
| 4 | 1 | Complement | 24 | 210 | GTGCGATTACTCGTTATTTACCTACAGAACACCGAG |
| 4 | 1 | Complement | 25 | 211 | TTGGGTACTAAAGGCCAGGTTCGCTTGGGATGTTAC |
| 4 | 1 | Complement | 26 | 212 | CCGTATTTATAATTCGCGACTGTTGCAGCACTACCC |
| 4 | 1 | Complement | 27 | 213 | GAGCTCACTGTGTATGTGTGTGATAAATGACTGACT |
| 4 | 1 | Complement | 28 | 214 | TGGTCCGAGATGCGAAGGGTGATGAGCATCTTTGAA |
| 4 | 1 | Complement | 29 | 215 | AGAAACTGTAAGAGGGACAGGCGCACTCGAGACTAT |
| 4 | 1 | Complement | 30 | 216 | GTTGCCAGTAAAGTAATACAGGTAGATCGCGTAGGA |
| 4 | 1 | Complement | 31 | 217 | CAAGAAGGATTGTGTGACGGATCAGCTTCCGCAGAA |
| 4 | 1 | Complement | 32 | 218 | CCGATAAGTATTGGTTGTATTCAAGCGGCGGAACCA |
| 4 | 1 | Complement | 33 | 219 | GAATTGAGTTGAAGCGTACTGCTCGTAAACGCTCAC |
| 4 | 1 | Complement | 34 | 220 | GTTCAGACGATTTGGCGTTCCAACAATAACACCCTG |
| 4 | 1 | Complement | 35 | 221 | CCAGCAAGGATTCGATGTTGAAAGTTCATTTGAGCG |
| 4 | 1 | Complement | 36 | 222 | ACTGCACAATTCCTAAGAAGCATCTGCCCAATACCA |
| 4 | 1 | Complement | 37 | 223 | CGCTGACCTAATCGTAAATTTCGTGCATGGCAAGAT |
| 4 | 1 | Complement | 38 | 224 | GTCCTGCGGAGAGCGGGTTCCTGTTTAAGTAGTATA |
| 4 | 1 | Complement | 39 | 225 | CCGTCAACAGACGCATTTCTGAATGTAGGGATCTAC |
| 4 | 1 | Complement | 40 | 226 | CAACTACTTGCTATTATATTCGCGCACTTAGCACAC |
| 4 | 1 | Complement | 41 | 227 | ATGATGTGTGCCTAACTAACTCATTATCTGCTTGCG |
| 4 | 1 | Complement | 42 | 228 | CTAGCCAATCTGTTACAAGCCTAGGAGCGTGATACT |
| 4 | 1 | Complement | 43 | 229 | GGTGGACAGTGTCACATAGATTGACGAATTGTATGG |
| 4 | 1 | Complement | 44 | 230 | GCTGAGTAACGTCTTATGTTGAGCAACCCGTCATGT |
| 4 | 1 | Complement | 45 | 231 | CAGGGACACTCATGTCGTTACGTTTATTAACCGCCG |
| 4 | 1 | Complement | 46 | 232 | CGCGGAACTAGGCAGGAATGTCGGAAATACTTGAAA |
| 4 | 1 | Complement | 47 | 233 | AAGACCTATAGCGACGTACGCTAGTCTAGTTACGAA |
| 4 | 1 | Complement | 48 | 234 | CAGTCAGTTGATTTATACGCGGCACCGAATTCCACT |
| 4 | 1 | Complement | 49 | 235 | TGTAGGAGAAGGTGTCTTGTGTAAGGATGAGGCAAA |
| 4 | 1 | Complement | 50 | 236 | ATACACGAAGATAAGGCAGGGTGGGTTGTTGATATC |
| 4 | 1 | Complement | 51 | 237 | CGTATGTACACGCGCAGCTAATTTAACTATATCTGG |
| 4 | 1 | Complement | 52 | 238 | ACGACTCATTGAAGGTCGCGGGACGGGTAATATTCA |
| 4 | 1 | Complement | 53 | 239 | CAAGCTTACGGAGGAATAACATGTCCCAATTGATGA |
| 4 | 1 | Complement | 54 | 240 | CCCGAATTACGCACGGCCCTTGACTTTGTTCTATAA |
| 4 | 1 | Complement | 55 | 241 | GTACATAGCTGATGTTAACTTAGGGCCTACTGAGGC |
| 4 | 1 | Complement | 56 | 242 | TGAGGCCAAGATGAATTACCACTCCATTGGCCTGCT |
| 4 | 1 | Complement | 57 | 243 | TTATGTCCGTCTGCTTTAGATATGAGCCGACACTTA |
| 4 | 1 | Complement | 58 | 244 | GTAAGTTCGACCGGCTCGAGGATCATCATATCAATG |
| 4 | 1 | Complement | 59 | 245 | GTAGTTTGATAAGCGTCGAAAGACGCCAGTACTTCC |
| 4 | 1 | Complement | 60 | 246 | ACGAATGCGCGCTATCTTATGGGTTTCTCGTATATT |
| 4 | 1 | Complement | 61 | 247 | TCCAACCTCCGCATTAAGACCCAGGTAGTAGCTGTT |
| 4 | 1 | Complement | 62 | 248 | TTTCAAATCCCACCGGCCTCATGTTAGATCAGTGGG |
| 4 | 1 | Complement | 63 | 249 | AATTCTTTATCAGGGCAGCGGTTAGTACGAGGTCGG |
| 4 | 1 | Complement | 64 | 250 | ACTATGATAGAGAGGCCCGGATCTTTGTCACGAACG |

TABLE 5

Capture Oligonucleotide set 5: 36-mer non-cross-reactive capture oligonucleotides complementary to the sequences generated using base oligonucleotide #2

| Run | Base | Transform | Parent SEQ ID NO | SEQ ID NO | Sequence |
|---|---|---|---|---|---|
| 5 | 2 | Complement | 65 | 251 | TTGATCATTCCTTTGATACGTACTCTGAACGACGAC |
| 5 | 2 | Complement | 66 | 252 | TTATGCAAAGACAGAGGATCTGGAGGCCGTGCTGTT |
| 5 | 2 | Complement | 67 | 253 | CCAGTAGCTAAGCGGAAAGACTCAGCGTTTCAGTAA |
| 5 | 2 | Complement | 68 | 254 | ACTCCGTATACCAATTGAATGGCTCGTTCGAAGAAT |
| 5 | 2 | Complement | 69 | 255 | GCATACCTACCTACAAATAGTGCACGCGCTCTCTAA |
| 5 | 2 | Complement | 70 | 256 | CACAACCGGCAAAGCTATTTAATCTCAAGACCAGAT |
| 5 | 2 | Complement | 71 | 257 | GGGACGAGCAGTACTTTGTGCGAACAGATTAAGCTA |
| 5 | 2 | Complement | 72 | 258 | GCCGCGAGAGCTTTAGTCGTAATTGAGAAATTGCGG |
| 5 | 2 | Complement | 73 | 259 | TGCAATGCCCAATGAGAGCTGAAATATAGTGCGGTC |
| 5 | 2 | Complement | 74 | 260 | GTCAGGTCCTCAGGAATAGAGTCTAAACAAGCCGCT |
| 5 | 2 | Complement | 75 | 261 | GGAGTGAAGGAAAGTGATTGGACTGTCGTTGGTATG |
| 5 | 2 | Complement | 76 | 262 | AATGAGAAAGCGAGTCGTTGCGGTGATTTCGATATA |
| 5 | 2 | Complement | 77 | 263 | CCGCTTGACATAGCCGTCCGGGATTTCTTTAAACAT |
| 5 | 2 | Complement | 78 | 264 | TCAGCGGTCTCCGATCAACACATGATAGTATAGCTG |
| 5 | 2 | Complement | 79 | 265 | AGATAGACACGTCGGATGATATACCACGCTGTTATT |
| 5 | 2 | Complement | 80 | 266 | AAGGAATTCGCTCGCTGCAGTTGACCTTCTACATAC |
| 5 | 2 | Complement | 81 | 267 | TACCTAGTGGAGGGACCTATACAAGTCAAATAGCGA |
| 5 | 2 | Complement | 82 | 268 | GTAGAGTTTGATAAGAATCGGGTACCAACCCACTTT |
| 5 | 2 | Complement | 83 | 269 | GACAGGTTGATGATGGTTCTAGACAACTAATGCGTC |
| 5 | 2 | Complement | 84 | 270 | ACCTTCCATGGCATTGACCCTGATGGTGTTAATAGT |
| 5 | 2 | Complement | 85 | 271 | TTCGCAGCCTATGCGTTCACATGGACCTAACTCGTA |
| 5 | 2 | Complement | 86 | 272 | ATATATACAGCGGCAACAGGTGAAAGACCTTGACTA |
| 5 | 2 | Complement | 87 | 273 | GTCGGCCTAAGCAATACTCGGGCGTGCTAATAATAT |
| 5 | 2 | Complement | 88 | 274 | ATCGACGATACCCGACACAACGCCACGTAGATTCAT |
| 5 | 2 | Complement | 89 | 275 | GGGCTAGCAATGTTTATTCGTCCACGGTGAGTAACT |
| 5 | 2 | Complement | 90 | 276 | TCATGCACATGTCTGTAGCTTCAAGTTTCGCGAGGG |
| 5 | 2 | Complement | 91 | 277 | ACCTGTCCATCCAGACAGTAAGTAGAGGAATCAGTC |
| 5 | 2 | Complement | 92 | 278 | GAGTCTAGCCAGATTGGTGTCATCCTGCCTGATTGT |
| 5 | 2 | Complement | 93 | 279 | TTCATTCTAGATCAAGGCTCCAAGGCTTACGCTCCA |
| 5 | 2 | Complement | 94 | 280 | CCACTCACTGGAAAGTTCTAAGGTTAAGACGCTTCG |
| 5 | 2 | Complement | 95 | 281 | TTTAGGCGACTGACGAACCAGAACCCTGAATGTTCG |
| 5 | 2 | Complement | 96 | 282 | TTGTATCTTGCTCCTCGGATGTAGAGCGATTGAGGA |
| 5 | 2 | Complement | 97 | 283 | ACTGATCGATTGTGTCCTCATTACCGTCCCAACACA |
| 5 | 2 | Complement | 98 | 284 | TGTGTAGCCTTCAGTGCTAACTTGTTTGAGTAAGCT |
| 5 | 2 | Complement | 99 | 285 | ATGTTTAGGACTCACAAGGTCGGTGCTGCAATTCGA |
| 5 | 2 | Complement | 100 | 286 | ATAATCACACGGATCACCATTCTCTCTCACTTCGCT |
| 5 | 2 | Complement | 101 | 287 | TTGTTCATGCCGCATACGATTAACCCTTCCGGTCCA |
| 5 | 2 | Complement | 102 | 288 | TGTATGCTGTCACGAGATTGAGTTAGAGTCCCTATT |
| 5 | 2 | Complement | 103 | 289 | GAGTTGTTTGTGAAGGTTTGACCATTTGACGAGCCT |
| 5 | 2 | Complement | 104 | 290 | TACGACCAAGTGTAAGTTCGTCGAGTAGAACGGTCC |
| 5 | 2 | Complement | 105 | 291 | TGAAGGGTGGTAAGAGGACATGTAGCAACTGTCGCA |
| 5 | 2 | Complement | 106 | 292 | TAGTGTATATTGAGCATCCAAAGCACGATTCACGGC |
| 5 | 2 | Complement | 107 | 293 | GAGATCCTGAACCTCGTTTCTCTGAGTAGCAGATAG |
| 5 | 2 | Complement | 108 | 294 | CTTGCGTTTGGGTGCCACATCAATAATCCCAGGATC |
| 5 | 2 | Complement | 109 | 295 | CTAAATTGATGCAACCTAAGAAAGCTGTGGTTCCGC |
| 5 | 2 | Complement | 110 | 296 | TATAGTAGACTAATGACGTGCATCTACCGCACAGAT |
| 5 | 2 | Complement | 111 | 297 | GAAGCACAGTCTGTTTCCCGAATCGAATAACGTCGG |
| 5 | 2 | Complement | 112 | 298 | TAATTAAACTGTGTTACGTTACTACCCAGCGCCAGA |
| 5 | 2 | Complement | 113 | 299 | TAGTGTTATCTTAACTGCGGTTGTTCTGCGCCCACT |
| 5 | 2 | Complement | 114 | 300 | TCTATGTTATATCTCATGTCGGTGTCCCTACGTACG |
| 5 | 2 | Complement | 115 | 301 | ACGAAACGCCGTTGTTGTTACGAAGACGGCGAAATG |
| 5 | 2 | Complement | 116 | 302 | AAGCATAATTCCAAACCCAGTCACTCCGTCACTCGT |
| 5 | 2 | Complement | 117 | 303 | GATAATTCTCATGCTACACGCACCGCGCTGAAGTAC |
| 5 | 2 | Complement | 118 | 304 | AACCGCATTGATAGTTCGTTACGTGTTCAAGAGCCA |
| 5 | 2 | Complement | 119 | 305 | TTGGGCCAGGCTCCGTTCTGGGATCTTATTGTTTAA |
| 5 | 2 | Complement | 120 | 306 | TGCTTCCTGACTTAGTGCAAGATGGAGAGTGTTCAT |
| 5 | 2 | Complement | 121 | 307 | CCGTGTCCTATAAAGTGGAATCACAGAATCTGAGGG |
| 5 | 2 | Complement | 122 | 308 | CGTATTGGTTCTCCTGCTGTTCACGTATATGATGTC |

TABLE 6

Capture Oligonucleotide set 6: 36-mer non-cross-reactive capture oligonucleotides complementary to the sequences generated using base oligonucleotide #3

| Run | Base | Transform | Parent SEQ ID NO | SEQ ID NO | Sequence |
|---|---|---|---|---|---|
| 6 | 3 | Complement | 123 | 309 | TGTGCTCCTAGATTCACAAACCCTCTCCACAGTAGC |
| 6 | 3 | Complement | 124 | 310 | TGGATGGTTATGTTTATTGCTCCCTAGCATTGCTGT |
| 6 | 3 | Complement | 125 | 311 | CGGTCCGAAATGATCCAAATGGAAAGAGAACGTCTC |
| 6 | 3 | Complement | 126 | 312 | CACCCAGTAAACGAGTATGGGCCACAACTTCTGGTT |
| 6 | 3 | Complement | 127 | 313 | CAAATCGTCGTACTCTGTGGTGGAGTTACGATTCGC |
| 6 | 3 | Complement | 128 | 314 | GTCAATCGTACTGAGCGAATATAAGGGCTACGACCG |
| 6 | 3 | Complement | 129 | 315 | CTGTTCTGAAACATCTAAACGGTCGCAAGCGGAACC |
| 6 | 3 | Complement | 130 | 316 | GACTATCTAGGTTTACGACGTATCACGCGAACAGCC |
| 6 | 3 | Complement | 131 | 317 | CGGTCATACAGGACGTGACAAATACTAGACATGGTT |
| 6 | 3 | Complement | 132 | 318 | TTAGGCAAACCACAAGGACAGGTGTGGCGAAGTACA |
| 6 | 3 | Complement | 133 | 319 | TATGCCATACATCTACCGGCTCTGAAGGACCCTAGT |
| 6 | 3 | Complement | 134 | 320 | TAGCTCGGCTACACACCGTCCCTATACCAGTGTAAA |
| 6 | 3 | Complement | 135 | 321 | ATGCGCGTCTCGTCCTCCAAATCATATTTAACCCAC |
| 6 | 3 | Complement | 136 | 322 | AGTTCCGTGGAAACTGCGAATAGCCGTCAGAACTTT |
| 6 | 3 | Complement | 137 | 323 | AAGACGATGCCGCTATTCTGCACACGAAGGCAATCT |
| 6 | 3 | Complement | 138 | 324 | AATTCCCGTTAAGACGAGCCCGTATCGCCTTGGAAT |
| 6 | 3 | Complement | 139 | 325 | TGTGTAGACAATAACGTCTGTCAGGCGAGGCTTATA |
| 6 | 3 | Complement | 140 | 326 | TTCTATAGCTAAGTGTAGATGGGCGTTAAGCCAGCA |
| 6 | 3 | Complement | 141 | 327 | TTGAGCTATTTACTATGTGCGGTTTATAACCTCCCG |
| 6 | 3 | Complement | 142 | 328 | TTACAGGTATTAGCTGCTGCTCACAGGCGAACTGAT |
| 6 | 3 | Complement | 143 | 329 | GTCTGTTCATCCCAGGTATACGTAGTTGATACCTTT |
| 6 | 3 | Complement | 144 | 330 | CGACGTTGTTCAAACTCATAGAAACTATCCCTGGAG |
| 6 | 3 | Complement | 145 | 331 | ATCCTTGGGTGCTTACAGACAGTTCGTCAAATAGAG |
| 6 | 3 | Complement | 146 | 332 | GAAAGTACGGCCGAGACACTTTCCTTCATATAACTC |
| 6 | 3 | Complement | 147 | 333 | CAAGACAGCCCTATATTTGTGGGTACACGACCAGAC |
| 6 | 3 | Complement | 148 | 334 | GGCCAAAGTAAAGACTGGTTTCGCTATCTGGATGTT |
| 6 | 3 | Complement | 149 | 335 | TTGCAGTTCAAGAAATGGTGGATCGAGCGCTTTAGT |
| 6 | 3 | Complement | 150 | 336 | GCAATCCTATGACGCCCAGTGTTAGACTACATGTCC |
| 6 | 3 | Complement | 151 | 337 | TAACAGCACAGTCCAGGTTGAAGCGAGTTCGAAGAT |
| 6 | 3 | Complement | 152 | 338 | GGCTACTCTTTGTAGTAATCGCAATTGAGCACGGGA |
| 6 | 3 | Complement | 153 | 339 | CATAGGAAGCAGTGGCAAACTCCGGCGAGAAATTGA |
| 6 | 3 | Complement | 154 | 340 | GGATACCCACTCTCCATGCGTCCATAGTATAGTGAT |
| 6 | 3 | Complement | 155 | 341 | CATTCAGCCAACGCTATTGCATCTAATCAAACCGTG |
| 6 | 3 | Complement | 156 | 342 | AACCTCGCCCACTCGGATAATGTATTCAGAGAGTAG |
| 6 | 3 | Complement | 157 | 343 | TAAAGGTTACGGGTTGCACAATCAACTTACCAGCGG |
| 6 | 3 | Complement | 158 | 344 | CAAGAGTGCATGCTCTTCTTATGTCGCTTTATTCCT |
| 6 | 3 | Complement | 159 | 345 | CCTGTGGGCTATATGTTCTCATCACTTAGGAGTCAG |
| 6 | 3 | Complement | 160 | 346 | ATAGCTTAAGTGAAGGCGTAGCACTAACGAACGATT |
| 6 | 3 | Complement | 161 | 347 | GGCGATGTGATTACAAATGGTCTCCGATACAGGTTG |
| 6 | 3 | Complement | 162 | 348 | GCTCTACTCCAGCTGAATCTTATTGCAAACGTTGAT |
| 6 | 3 | Complement | 163 | 349 | TCCTTCGAATCTAATGACCCATTACCGGAGCATAGT |
| 6 | 3 | Complement | 164 | 350 | CGACGTTAATAGTGGACAAAGCATTCGTCGATGTAG |
| 6 | 3 | Complement | 165 | 351 | ACTCCCGAAAGCAAATCAGTTATCACACCACGATAA |
| 6 | 3 | Complement | 166 | 352 | AAGAACTACCCAACCCGATAGAGTCACTGTAGATCC |
| 6 | 3 | Complement | 167 | 353 | CTTACAACTATGTCCTACAGCCATCCGGTCAGGAAT |
| 6 | 3 | Complement | 168 | 354 | TGGATTTGTCCTGTTAGCGTACCTCAGCGTCAGCAT |
| 6 | 3 | Complement | 169 | 355 | CGGAGGAATGACTTAACAGATATGGCCGCTAATTGT |
| 6 | 3 | Complement | 170 | 356 | CATCAGGATCCAACTACTCCGAGGATTCTTTATGCA |
| 6 | 3 | Complement | 171 | 357 | TAGTTAAGCTTCCCGCAATGTTTAACTGGCCTGTCC |
| 6 | 3 | Complement | 172 | 358 | AGTGAGATCTATTAGGGATTAACCACCGGGCGCAAA |
| 6 | 3 | Complement | 173 | 359 | CAGATTACCAACGCCAGCAATTTGCAGACACCAACC |
| 6 | 3 | Complement | 174 | 360 | GCGGAATTCACATATTGACAGCCGGAGATACAGCGT |
| 6 | 3 | Complement | 175 | 361 | TAGAGTAAGAGAGCACCGACTGCAGACTAAAGTGGA |
| 6 | 3 | Complement | 176 | 362 | TAATGCTCGGCGGATCAATGGGAAGCCTTAATTACT |
| 6 | 3 | Complement | 177 | 363 | ACCTAAGGTCTGCTGTGCTACCGAAGGTATATTCTG |
| 6 | 3 | Complement | 178 | 364 | TAACCCGTTTGAAAGCGCGATGATCAACAGTTTCGG |
| 6 | 3 | Complement | 179 | 365 | GAATTTGCTAGCTCCCAGACAAGATCGCCGAGTCAT |
| 6 | 3 | Complement | 180 | 366 | TTAGAGGGCCATACCAACACTTTAACGTAGTGACCC |
| 6 | 3 | Complement | 181 | 367 | CGATCTGATTCGGCACGGATCCATAACGGTGTTGAA |
| 6 | 3 | Complement | 182 | 368 | CAGGATGTGGAAGTGCCGATACTCCATACAGACTGA |
| 6 | 3 | Complement | 183 | 369 | CCTAATTGCACGAGATCCAAGCTACTATTGCCCGCA |
| 6 | 3 | Complement | 184 | 370 | GTCTCCAGGAAATGTGAGCGACGGTAAACCGGAATT |
| 6 | 3 | Complement | 185 | 371 | TCCCGTAGCATCCGCCTAATTTCGCACAACACAACT |
| 6 | 3 | Complement | 186 | 372 | ATCGAGACATCATTAACACACTCAGCCAGGCAACGT |

TABLE 7

Capture Oligonucleotide set 7: 36-mer non-cross-reactive capture oligonucleotides having sequences that are the reverse of the sequences generated using base oligonucleotide #1

| Run | Base | Transform | Parent SEQ ID NO | SEQ ID NO | Sequence |
|---|---|---|---|---|---|
| 7 | 1 | Reverse | 1 | 373 | CTCTGTGCTGATTGACCATTGGGTCTGTACTAGCCA |
| 7 | 1 | Reverse | 2 | 374 | TACCTCCCGAACGTAAACCAGTAACCAAGTTCTGCT |
| 7 | 1 | Reverse | 3 | 375 | GGAAAGCGATGTGATTATCGAGGACACGGGAGCATA |
| 7 | 1 | Reverse | 4 | 376 | ATTTGACTACGCCACCGTTGTCTTTAGTCCACCACT |
| 7 | 1 | Reverse | 5 | 377 | TGTACAGTACTTATCTACGGCGACGTCCATACCTTT |
| 7 | 1 | Reverse | 6 | 378 | CAAATCTGAGGTTGAGGGACACCCACTCGAATTACC |
| 7 | 1 | Reverse | 7 | 379 | ATATACGTGAGAGACCAGGCAACACCAGGAAGATTG |
| 7 | 1 | Reverse | 8 | 380 | GCTCACTGCCATTTAGGCAGCTGATCGGACTCTTAA |
| 7 | 1 | Reverse | 9 | 381 | CGTCTCTGGTTGTCTCCTGACGATTTCCAAACATCC |
| 7 | 1 | Reverse | 10 | 382 | CTCAGCCTACCAGCATGGTAACTTAGACCTCTGGAA |
| 7 | 1 | Reverse | 11 | 383 | GAGCCTTCCTCTTAAGGAGTGTCTCTTGTCTGTCAA |
| 7 | 1 | Reverse | 12 | 384 | TTGCGACCCTCTGTAATTTGCTGTTACCAATGCACT |
| 7 | 1 | Reverse | 13 | 385 | TTGGCAAGTAGCAGCCCTATCGTAGTAGACCGTATC |
| 7 | 1 | Reverse | 14 | 386 | TTCTGGTGTGAGCTTATCCATCAACACTGCATCCCT |
| 7 | 1 | Reverse | 15 | 387 | CGGATATTTCTCGGGCGAACCAATCGATCTATCGTG |
| 7 | 1 | Reverse | 16 | 388 | AGATGCGCAGCCGTGATACCGTCATTGGGAATCATA |
| 7 | 1 | Reverse | 17 | 389 | ACGGACACCTATGAACCACAGATCTTTCTTTGTGGA |
| 7 | 1 | Reverse | 18 | 390 | GAGGCTAATTGACTTTCGTTTGCAACGAGGCAGAGC |
| 7 | 1 | Reverse | 19 | 391 | GGATGGTAGAGGTTTGTGAAATACATTCTAAGGCAG |
| 7 | 1 | Reverse | 20 | 392 | TAGGCGTAGCAGATGGGCTGAATTTAGTGCCATGAC |
| 7 | 1 | Reverse | 21 | 393 | GAGTCTCTTATGGGTTTAGGCCATATTGCATCTCTG |
| 7 | 1 | Reverse | 22 | 394 | TCAATGGCTTGGCGAAAGGGCGTCAGAGTTTCTTTC |
| 7 | 1 | Reverse | 23 | 395 | TATACTTTGCCCGTGACTCGGTTCGGCACATTCATG |
| 7 | 1 | Reverse | 24 | 396 | CACGCTAATGAGCAATAAATGGATGTCTTGTGGCTC |
| 7 | 1 | Reverse | 25 | 397 | AACCCATGATTTCCGGTCCAAGCGAACCCTACAATG |
| 7 | 1 | Reverse | 26 | 398 | GGCATAAATATTAAGCGCTGACAACGTCGTGATGGG |
| 7 | 1 | Reverse | 27 | 399 | CTCGAGTGACACATACACACACTATTTACTGACTGA |
| 7 | 1 | Reverse | 28 | 400 | ACCAGGCTCTACGCTTCCCACTACTCGTAGAAACTT |
| 7 | 1 | Reverse | 29 | 401 | TCTTTGACATTCTCCCTGTCCGCGTGAGCTCTGATA |
| 7 | 1 | Reverse | 30 | 402 | CAACGGTCATTTCATTATGTCCATCTAGCGCATCCT |
| 7 | 1 | Reverse | 31 | 403 | GTTCTTCCTAACACACTGCCTAGTCGAAGGCGTCTT |
| 7 | 1 | Reverse | 32 | 404 | GGCTATTCATAACCAACATAAGTTCGCCGCCTTGGT |
| 7 | 1 | Reverse | 33 | 405 | CTTAACTCAACTTCGCATGACGAGCATTTGCGAGTG |
| 7 | 1 | Reverse | 34 | 406 | CAAGTCTGCTAAACCGCAAGGTTGTTATTGTGGGAC |
| 7 | 1 | Reverse | 35 | 407 | GGTCGTTCCTAAGCTACAACTTTCAAGTAAACTCGC |
| 7 | 1 | Reverse | 36 | 408 | TGACGTGTTAAGGATTCTTCGTAGACGGGTTATGGT |
| 7 | 1 | Reverse | 37 | 409 | GCGACTGGATTAGCATTTAAAGCACGTACCGTTCTA |
| 7 | 1 | Reverse | 38 | 410 | CAGGACGCCTCTCGCCCAAGGACAAATTCATCATAT |
| 7 | 1 | Reverse | 39 | 411 | GGCAGTTGTCTGCGTAAAGACTTACATCCCTAGATG |
| 7 | 1 | Reverse | 40 | 412 | GTTGATGAACGATAATATAAGCGCGTGAATCGTGTG |
| 7 | 1 | Reverse | 41 | 413 | TACTACACACGGATTGATTGAGTAATAGACGAACGC |
| 7 | 1 | Reverse | 42 | 414 | GATCGGTTAGACAATGTTCGGATCCTCGCACTATGA |
| 7 | 1 | Reverse | 43 | 415 | CCACCTGTCACAGTGTATCTAACTGCTTAACATACC |
| 7 | 1 | Reverse | 44 | 416 | CGACTCATTGCAGAATACAACTCGTTGGGCAGTACA |
| 7 | 1 | Reverse | 45 | 417 | GTCCCTGTGAGTACAGCAATGCAAATAATTGGCGGC |
| 7 | 1 | Reverse | 46 | 418 | GCGCCTTGATCCGTCCTTACAGCCTTTATGAACTTT |
| 7 | 1 | Reverse | 47 | 419 | TTCTGGATATCGCTGCATGCGATCAGATCAATGCTT |
| 7 | 1 | Reverse | 48 | 420 | GTCAGTCAACTAAATATGCGCCGTGGCTTAAGGTGA |
| 7 | 1 | Reverse | 49 | 421 | ACATCCTCTTCCACAGAACACATTCCTACTCCGTTT |
| 7 | 1 | Reverse | 50 | 422 | TATGTGCTTCTATTCCGTCCCACCCAACAACTATAG |
| 7 | 1 | Reverse | 51 | 423 | GCATACATGTGCGCGTCGATTAAATTGATATAGACC |
| 7 | 1 | Reverse | 52 | 424 | TGCTGAGTAACTTCCAGCGCCCTGCCCATTATAAGT |
| 7 | 1 | Reverse | 53 | 425 | GTTCGAATGCCTCCTTATTGTACAGGGTTAACTACT |
| 7 | 1 | Reverse | 54 | 426 | GGGCTTAATGCGTGCCGGGAACTGAAACAAGATATT |
| 7 | 1 | Reverse | 55 | 427 | CATGTATCGACTACAATTGAATCCCGGATGACTCCG |
| 7 | 1 | Reverse | 56 | 428 | ACTCCGGTTCTACTTAATGGTGAGGTAACCGGACGA |
| 7 | 1 | Reverse | 57 | 429 | AATACAGGCAGACGAAATCTATACTCGGCTGTGAAT |
| 7 | 1 | Reverse | 58 | 430 | CATTCAAGCTGGCCGAGCTCCTAGTAGTATAGTTAC |
| 7 | 1 | Reverse | 59 | 431 | CATCAAACTATTCGCAGCTTTCTGCGGTCATGAAGG |
| 7 | 1 | Reverse | 60 | 432 | TGCTTACGCGCGATAGAATACCCAAAGAGCATATAA |
| 7 | 1 | Reverse | 61 | 433 | AGGTTGGAGGCGTAATTCTGGGTCCATCATCGACAA |
| 7 | 1 | Reverse | 62 | 434 | AAAGTTTAGGGTGGCCGGAGTACAATCTAGTCACCC |
| 7 | 1 | Reverse | 63 | 435 | TTAAGAAATAGTCCCGTCGCCAATCATGCTCCAGCC |
| 7 | 1 | Reverse | 64 | 436 | TGATACTATCTCTCCGGGCCTAGAAACAGTGCTTGC |

TABLE 8

Capture Oligonucleotide set 8: 36-mer non-cross-reactive capture oligonucleotides having sequences that are the reverse of the sequences generated using base oligonucleotide #2

| Run | Base | Transform | Parent SEQ ID NO | SEQ ID NO | Sequence |
|---|---|---|---|---|---|
| 8 | 2 | Reverse | 65 | 437 | AACTAGTAAGGAAACTATGCATGAGACTTGCTGCTG |
| 8 | 2 | Reverse | 66 | 438 | AATACGTTTCTGTCTCCTAGACCTCCGGCACGACAA |
| 8 | 2 | Reverse | 67 | 439 | GGTCATCGATTCGCCTTTCTGAGTCGCAAAGTCATT |
| 8 | 2 | Reverse | 68 | 440 | TGAGGCATATGGTTAACTTACCGAGCAAGCTTCTTA |
| 8 | 2 | Reverse | 69 | 441 | CGTATGGATGGATGTTTATCACGTGCGCGAGAGATT |
| 8 | 2 | Reverse | 70 | 442 | GTGTTGGCCGTTTCGATAAATTAGAGTTCTGGTCTA |
| 8 | 2 | Reverse | 71 | 443 | CCCTGCTCGTCATGAAACACGCTTGTCTAATTCGAT |
| 8 | 2 | Reverse | 72 | 444 | CGGCGCTCTCGAAATCAGCATTAACTCTTTAACGCC |
| 8 | 2 | Reverse | 73 | 445 | ACGTTACGGGTTACTCTCGACTTTATATCACGCCAG |
| 8 | 2 | Reverse | 74 | 446 | CAGTCCAGGAGTCCTTATCTCAGATTTGTTCGGCGA |
| 8 | 2 | Reverse | 75 | 447 | CCTCACTTCCTTTCACTAACCTGACAGCAACCATAC |
| 8 | 2 | Reverse | 76 | 448 | TTACTCTTTCGCTCAGCAACGCCACTAAAGCTATAT |
| 8 | 2 | Reverse | 77 | 449 | GGCGAACTGTATCGGCAGGCCCTAAAGAAATTTGTA |
| 8 | 2 | Reverse | 78 | 450 | AGTCGCCAGAGGCTAGTTGTGTACTATCATATCGAC |
| 8 | 2 | Reverse | 79 | 451 | TCTATCTGTGCAGCCTACTATATGGTGCGACAATAA |
| 8 | 2 | Reverse | 80 | 452 | TTCCTTAAGCGAGCGACGTCAACTGGAAGATGTATG |
| 8 | 2 | Reverse | 81 | 453 | ATGGATCACCTCCCTGGATATGTTCAGTTTATCGCT |
| 8 | 2 | Reverse | 82 | 454 | CATCTCAAACTATTCTTAGCCCATGGTTGGGTGAAA |
| 8 | 2 | Reverse | 83 | 455 | CTGTCCAACTACTACCAAGATCTGTTGATTACGCAG |
| 8 | 2 | Reverse | 84 | 456 | TGGAAGGTACCGTAACTGGGACTACCACAATTATCA |
| 8 | 2 | Reverse | 85 | 457 | AAGCGTCGGATACGCAAGTGTACCTGGATTGAGCAT |
| 8 | 2 | Reverse | 86 | 458 | TATATATGTCGCCGTTGTCCACTTTCTGGAACTGAT |
| 8 | 2 | Reverse | 87 | 459 | CAGCCGGATTCGTTATGAGCCCGCACGATTATTATA |
| 8 | 2 | Reverse | 88 | 460 | TAGCTGCTATGGGCTGTGTTGCGGTGCATCTAAGTA |
| 8 | 2 | Reverse | 89 | 461 | CCCGATCGTTACAAATAAGCAGGTGCCACTCATTGA |
| 8 | 2 | Reverse | 90 | 462 | AGTACGTGTACAGACATCGAAGTTCAAAGCGCTCCC |
| 8 | 2 | Reverse | 91 | 463 | TGGACAGGTAGGTCTGTCATTCATCTCCTTAGTCAG |
| 8 | 2 | Reverse | 92 | 464 | CTCAGATCGGTCTAACCACAGTAGGACGGACTAACA |
| 8 | 2 | Reverse | 93 | 465 | AAGTAAGATCTAGTTCCGAGGTTCCGAATGCGAGGT |
| 8 | 2 | Reverse | 94 | 466 | GGTGAGTGACCTTTCAAGATTCCAATTCTGCGAAGC |
| 8 | 2 | Reverse | 95 | 467 | AAATCCGCTGACTGCTTGGTCTTGGGACTTACAAGC |
| 8 | 2 | Reverse | 96 | 468 | AACATAGAACGAGGAGCCTACATCTCGCTAACTCCT |
| 8 | 2 | Reverse | 97 | 469 | TGACTAGCTAACACAGGAGTAATGGCAGGGTTGTGT |
| 8 | 2 | Reverse | 98 | 470 | ACACATCGGAAGTCACGATTGAACAAACTCATTCGA |
| 8 | 2 | Reverse | 99 | 471 | TACAAATCCTGAGTGTTCCAGCCACGACGTTAAGCT |
| 8 | 2 | Reverse | 100 | 472 | TATTAGTGTGCCTAGTGGTAAGAGAGAGTGAAGCGA |
| 8 | 2 | Reverse | 101 | 473 | AACAAGTACGGCGTATGCTAATTGGGAAGGCCAGGT |
| 8 | 2 | Reverse | 102 | 474 | ACATACGACAGTGCTCTAACTCAATCTCAGGGATAA |
| 8 | 2 | Reverse | 103 | 475 | CTCAACAAACACTTCCAAACTGGTAAACTGCTCGGA |
| 8 | 2 | Reverse | 104 | 476 | ATGCTGGTTCACATTCAAGCAGCTCATCTTGCCAGG |
| 8 | 2 | Reverse | 105 | 477 | ACTTCCCACCATTCTCCTGTACATCGTTGACAGCGT |
| 8 | 2 | Reverse | 106 | 478 | ATCACATATAACTCGTAGGTTTCGTGCTAAGTGCCG |
| 8 | 2 | Reverse | 107 | 479 | CTCTAGGACTTGGAGCAAAGAGACTCATCGTCTATC |
| 8 | 2 | Reverse | 108 | 480 | GAACGCAAACCCACGGTGTAGTTATTAGGGTCCTAG |
| 8 | 2 | Reverse | 109 | 481 | GATTTAACTACGTTGGATTCTTTCGACACCAAGGCG |
| 8 | 2 | Reverse | 110 | 482 | ATATCATCTGATTACTGCACGTAGATGGCGTGTCTA |
| 8 | 2 | Reverse | 111 | 483 | CTTCGTGTCAGACAAAGGGCTTAGCTTATTGCAGCC |
| 8 | 2 | Reverse | 112 | 484 | ATTAATTTGACACAATGCAATGATGGGTCGCGGTCT |
| 8 | 2 | Reverse | 113 | 485 | ATCACAATAGAATTGACGCCAACAAGACGCGGGTGA |
| 8 | 2 | Reverse | 114 | 486 | AGATACAATATAGAGTACAGCCACAGGGATGCATGC |
| 8 | 2 | Reverse | 115 | 487 | TGCTTTGCGGCAACAACAATGCTTCTGCCGCTTTAC |
| 8 | 2 | Reverse | 116 | 488 | TTCGTATTAAGGTTTGGGTCAGTGAGGCAGTGAGCA |
| 8 | 2 | Reverse | 117 | 489 | CTATTAAGAGTACGATGTGCGTGGCGCGACTTCATG |
| 8 | 2 | Reverse | 118 | 490 | TTGGCGTAACTATCAAGCAATGCACAAGTTCTCGGT |
| 8 | 2 | Reverse | 119 | 491 | AACCCGGTCCGAGGCAAGACCCTAGAATAACAAATT |
| 8 | 2 | Reverse | 120 | 492 | ACGAAGGACTGAATCACGTTCTACCTCTCACAAGTA |
| 8 | 2 | Reverse | 121 | 493 | GGCACAGGATATTTCACCTTAGTGTCTTAGACTCCC |
| 8 | 2 | Reverse | 122 | 494 | GCATAACCAAGAGGACGACAAGTGCATATACTACAG |

TABLE 9

Capture Oligonucleotide set 9: 36-mer non-cross-reactive capture oligonucleotides having sequences that are the reverse of the sequences generated using base oligonucleotide #3

| Run | Base | Transform | Parent SEQ ID NO | SEQ ID NO | Sequence |
|---|---|---|---|---|---|
| 9 | 3 | Reverse | 123 | 495 | ACACGAGGATCTAAGTGTTTGGGAGAGGTGTCATCG |
| 9 | 3 | Reverse | 124 | 496 | ACCTACCAATACAAATAACGAGGGATCGTAACGACA |
| 9 | 3 | Reverse | 125 | 497 | GCCAGGCTTTACTAGGTTTACCTTTCTCTTGCAGAG |
| 9 | 3 | Reverse | 126 | 498 | GTGGGTCATTTGCTCATACCCGGTGTTGAAGACCAA |
| 9 | 3 | Reverse | 127 | 499 | GTTTAGCAGCATGAGACACCACCTCAATGCTAAGCG |
| 9 | 3 | Reverse | 128 | 500 | CAGTTAGCATGACTCGCTTATATTCCCGATGCTGGC |
| 9 | 3 | Reverse | 129 | 501 | GACAAGACTTTGTAGATTTGCCAGCGTTCGCCTTGG |
| 9 | 3 | Reverse | 130 | 502 | CTGATAGATCCAAATGCTGCATAGTGCGCTTGTCGG |
| 9 | 3 | Reverse | 131 | 503 | GCCAGTATGTCCTGCACTGTTTATGATCTGTACCAA |
| 9 | 3 | Reverse | 132 | 504 | AATCCGTTTGGTGTTCCTGTCCACACCGCTTCATGT |
| 9 | 3 | Reverse | 133 | 505 | ATACGGTATGTAGATGGCCGAGACTTCCTGGGATCA |
| 9 | 3 | Reverse | 134 | 506 | ATCGAGCCGATGTGTGGCAGGGATATGGTCACATTT |
| 9 | 3 | Reverse | 135 | 507 | TACGCGCAGAGCAGGAGGTTTAGTATAAATTGGGTG |
| 9 | 3 | Reverse | 136 | 508 | TCAAGGCACCTTTGACGCTTATCGGCAGTCTTGAAA |
| 9 | 3 | Reverse | 137 | 509 | TTCTGCTACGGCGATAAGACGTGTGCTTCCGTTAGA |
| 9 | 3 | Reverse | 138 | 510 | TTAAGGGCAATTCTGCTCGGGCATAGCGGAACCTTA |
| 9 | 3 | Reverse | 139 | 511 | ACACATCTGTTATTGCAGACAGTCCGCTCCGAATAT |
| 9 | 3 | Reverse | 140 | 512 | AAGATATCGATTCACATCTACCCGCAATTCGGTCGT |
| 9 | 3 | Reverse | 141 | 513 | AACTCGATAAATGATACACGCCAAATATTGGAGGGC |
| 9 | 3 | Reverse | 142 | 514 | AATGTCCATAATCGACGACGAGTGTCCGCTTGACTA |
| 9 | 3 | Reverse | 143 | 515 | CAGACAAGTAGGGTCCATATGCATCAACTATGGAAA |
| 9 | 3 | Reverse | 144 | 516 | GCTGCAACAAGTTTGAGTATCTTTGATAGGGACCTC |
| 9 | 3 | Reverse | 145 | 517 | TAGGAACCCACGAATGTCTGTCAAGCAGTTTATCTC |
| 9 | 3 | Reverse | 146 | 518 | CTTTCATGCCGGCTCTGTGAAAGGAAGTATATTGAG |
| 9 | 3 | Reverse | 147 | 519 | GTTCTGTCGGGATATAAACACCCATGTGCTGGTCTG |
| 9 | 3 | Reverse | 148 | 520 | CCGGTTTCATTTCTGACCAAAGCGATAGACCTACAA |
| 9 | 3 | Reverse | 149 | 521 | AACGTCAAGTTCTTTACCACCTAGCTCGCGAAATCA |
| 9 | 3 | Reverse | 150 | 522 | CGTTAGGATACTGCGGGTCACAATCTGATGTACAGG |
| 9 | 3 | Reverse | 151 | 523 | ATTGTCGTGTCAGGTCCAACTTCGCTCAAGCTTCTA |
| 9 | 3 | Reverse | 152 | 524 | CCGATGAGAAACATCATTAGCGTTAACTCGTGCCCT |
| 9 | 3 | Reverse | 153 | 525 | GTATCCTTCGTCACCGTTTGAGGCCGCTCTTTAACT |
| 9 | 3 | Reverse | 154 | 526 | CCTATGGGTGAGAGGTACGCAGGTATCATATCACTA |
| 9 | 3 | Reverse | 155 | 527 | GTAAGTCGGTTGCGATAACGTAGATTAGTTTGGCAC |
| 9 | 3 | Reverse | 156 | 528 | TTGGAGCGGGTGAGCCTATTACATAAGTCTCTCATC |
| 9 | 3 | Reverse | 157 | 529 | ATTTCCAATGCCCAACGTGTTAGTTGAATGGTCGCC |
| 9 | 3 | Reverse | 158 | 530 | GTTCTCACGTACGAGAAGAATACAGCGAAATAAGGA |
| 9 | 3 | Reverse | 159 | 531 | GGACACCCGATATACAAGAGTAGTGAATCCTCAGTC |
| 9 | 3 | Reverse | 160 | 532 | TATCGAATTCACTTCCGCATCGTGATTGCTTGCTAA |
| 9 | 3 | Reverse | 161 | 533 | CCGCTACACTAATGTTTACCAGAGGCTATGTCCAAC |
| 9 | 3 | Reverse | 162 | 534 | CGAGATGAGGTCGACTTAGAATAACGTTTGCAACTA |
| 9 | 3 | Reverse | 163 | 535 | AGGAAGCTTAGATTACTGGGTAATGGCCTCGTATCA |
| 9 | 3 | Reverse | 164 | 536 | GCTGCAATTATCACCTGTTTCGTAAGCAGCTACATC |
| 9 | 3 | Reverse | 165 | 537 | TGAGGGCTTTCGTTTAGTCAATAGTGTGGTGCTATT |
| 9 | 3 | Reverse | 166 | 538 | TTCTTGATGGGTTGGGCTATCTCAGTGACATCTAGG |
| 9 | 3 | Reverse | 167 | 539 | GAATGTTGATACAGGATGTCGGTAGGCCAGTCCTTA |
| 9 | 3 | Reverse | 168 | 540 | ACCTAAACAGGACAATCGCATGGAGTCGCAGTCGTA |
| 9 | 3 | Reverse | 169 | 541 | GCCTCCTTACTGAATTGTCTATACCGGCGATTAACA |
| 9 | 3 | Reverse | 170 | 542 | GTAGTCCTAGGTTGATGAGGCTCCTAAGAAATACGT |
| 9 | 3 | Reverse | 171 | 543 | ATCAATTCGAAGGGCGTTACAAATTGACCGGACAGG |
| 9 | 3 | Reverse | 172 | 544 | TCACTCTAGATAATCCCTAATTGGTGGCCCGCGTTT |
| 9 | 3 | Reverse | 173 | 545 | GTCTAATGGTTGCGGTCGTTAAACGTCTGTGGTTGG |
| 9 | 3 | Reverse | 174 | 546 | CGCCTTAAGTGTATAACTGTCGGCCTCTATGTCGCA |
| 9 | 3 | Reverse | 175 | 547 | ATCTCATTCTCTCGTGGCTGACGTCTGATTTCACCT |
| 9 | 3 | Reverse | 176 | 548 | ATTACGAGCCGCCTAGTTACCCTTCGGAATTAATGA |
| 9 | 3 | Reverse | 177 | 549 | TGGATTCCAGACGACACGATGGCTTCCATATAAGAC |
| 9 | 3 | Reverse | 178 | 550 | ATTGGGCAAACTTTCGCGCTACTAGTTGTCAAAGCC |
| 9 | 3 | Reverse | 179 | 551 | CTTAAACGATCGAGGGTCTGTTCTAGCGGCTCAGTA |
| 9 | 3 | Reverse | 180 | 552 | AATCTCCCGGTATGGTTGTGAAATTGCATCACTGGG |
| 9 | 3 | Reverse | 181 | 553 | GCTAGACTAAGCCGTGCCTAGGTATTGCCACAACTT |
| 9 | 3 | Reverse | 182 | 554 | GTCCTACACCTTCACGGCTATGAGGTATGTCTGACT |
| 9 | 3 | Reverse | 183 | 555 | GGATTAACGTGCTCTAGGTTCGATGATAACGGGCGT |
| 9 | 3 | Reverse | 184 | 556 | CAGAGGTCCTTTACACTCGCTGCCATTTGGCCTTAA |
| 9 | 3 | Reverse | 185 | 557 | AGGGCATCGTAGGCGGATTAAAGCGTGTTGTGTTGA |
| 9 | 3 | Reverse | 186 | 558 | TAGCTCTGTAGTAATTGTGTGAGTCGGTCCGTTGCA |

TABLE 10

Capture Oligonucleotide set 10: 36-mer non-cross-reactive capture oligonucleotides having sequences that are the inverse complement of the sequences generated using base oligonucleotide #1

| Run | Base | Transform | Parent SEQ ID NO | SEQ ID NO | Sequence |
|---|---|---|---|---|---|
| 10 | 1 | Inverse complement | 1 | 559 | TGGCTAGTACAGACCCAATGGTCAATCAGCACAGAG |
| 10 | 1 | Inverse complement | 2 | 560 | AGCAGAACTTGGTTACTGGTTTACGTTCGGGAGGTA |
| 10 | 1 | Inverse complement | 3 | 561 | TATGCTCCCGTGTCCTCGATAATCACATCGCTTTCC |
| 10 | 1 | Inverse complement | 4 | 562 | AGTGGTGGACTAAAGACAACGGTGGCGTAGTCAAAT |
| 10 | 1 | Inverse complement | 5 | 563 | AAAGGTATGGACGTCGCCGTAGATAAGTACTGTACA |
| 10 | 1 | Inverse complement | 6 | 564 | GGTAATTCGAGTGGGTGTCCCTCAACCTCAGATTTG |
| 10 | 1 | Inverse complement | 7 | 565 | CAATCTTCCTGGTGTTGCCTGGTCTCTCACGTATAT |
| 10 | 1 | Inverse complement | 8 | 566 | TTAAGAGTCCGATCAGCTGCCTAAATGGCAGTGAGC |
| 10 | 1 | Inverse complement | 9 | 567 | GGATGTTTGGAAATCGTCAGGAGACAACCAGAGACG |
| 10 | 1 | Inverse complement | 10 | 568 | TTCCAGAGGTCTAAGTTACCATGCTGGTAGGCTGAG |
| 10 | 1 | Inverse complement | 11 | 569 | TTGACAGACAAGAGACACTCCTTAAGAGGAAGGCTC |
| 10 | 1 | Inverse complement | 12 | 570 | AGTGCATTGGTAACAGCAAATTACAGAGGGTCGCAA |
| 10 | 1 | Inverse complement | 13 | 571 | GATACGGTCTACTACGATAGGGCTGCTACTTGCCAA |
| 10 | 1 | Inverse complement | 14 | 572 | AGGGATGCAGTGTTGATGGATAAGCTCACACCAGAA |
| 10 | 1 | Inverse complement | 15 | 573 | CACGATAGATCGATTGGTTCGCCCGAGAAATATCCG |
| 10 | 1 | Inverse complement | 16 | 574 | TATGATTCCCAATGACGGTATCACGGCTGCGCATCT |
| 10 | 1 | Inverse complement | 17 | 575 | TCCACAAAGAAAGATCTGTGGTTCATAGGTGTCCGT |
| 10 | 1 | Inverse complement | 18 | 576 | GCTCTGCCTCGTTGCAAACGAAAGTCAATTAGCCTC |
| 10 | 1 | Inverse complement | 19 | 577 | CTGCCTTAGAATGTATTTCACAAACCTCTACCATCC |
| 10 | 1 | Inverse complement | 20 | 578 | GTCATGGCACTAAATTCAGCCCATCTGCTACGCCTA |
| 10 | 1 | Inverse complement | 21 | 579 | CAGAGATGCAATATGGCCTAAACCCATAAGAGACTC |
| 10 | 1 | Inverse complement | 22 | 580 | GAAAGAAACTCTGACGCCCTTTCGCCAAGCCATTGA |
| 10 | 1 | Inverse complement | 23 | 581 | CATGAATGTGCCGAACCGAGTCACGGGCAAAGTATA |
| 10 | 1 | Inverse complement | 24 | 582 | GAGCCACAAGACATCCATTTATTGCTCATTAGCGTG |
| 10 | 1 | Inverse complement | 25 | 583 | CATTGTAGGGTTCGCTTGGACCGGAAATCATGGGTT |
| 10 | 1 | Inverse complement | 26 | 584 | CCCATCACGACGTTGTCAGCGCTTAATATTTATGCC |
| 10 | 1 | Inverse complement | 27 | 585 | TCAGTCAGTAAATAGTGTGTGTATGTGTCACTCGAG |
| 10 | 1 | Inverse complement | 28 | 586 | AAGTTTCTACGAGTAGTGGGAAGCGTAGAGCCTGGT |
| 10 | 1 | Inverse complement | 29 | 587 | TATCAGAGCTCACGCGGACAGGGAGAATGTCAAAGA |
| 10 | 1 | Inverse complement | 30 | 588 | AGGATGCGCTAGATGGACATAATGAAATGACCGTTG |
| 10 | 1 | Inverse complement | 31 | 589 | AAGACGCCTTCGACTAGGCAGTGTGTTAGGAAGAAC |
| 10 | 1 | Inverse complement | 32 | 590 | ACCAAGGCGGCGAACTTATGTTGGTTATGAATAGCC |
| 10 | 1 | Inverse complement | 33 | 591 | CACTCGCAAATGCTCGTCATGCGAAGTTGAGTTAAG |
| 10 | 1 | Inverse complement | 34 | 592 | GTCCCACAATAACAACCTTGCGGTTTAGCAGACTTG |
| 10 | 1 | Inverse complement | 35 | 593 | GCGAGTTTACTTGAAAGTTGTAGCTTAGGAACGACC |
| 10 | 1 | Inverse complement | 36 | 594 | ACCATAACCCGTCTACGAAGAATCCTTAACACGTCA |
| 10 | 1 | Inverse complement | 37 | 595 | TAGAACGGTACGTGCTTTAAATGCTAATCCAGTCGC |
| 10 | 1 | Inverse complement | 38 | 596 | ATATGATGAATTTGTCCTTGGGCGAGAGGCGTCCTG |
| 10 | 1 | Inverse complement | 39 | 597 | CATCTAGGGATGTAAGTCTTTACGCAGACAACTGCC |
| 10 | 1 | Inverse complement | 40 | 598 | CACACGATTCACGCGCTTATATTATCGTTCATCAAC |
| 10 | 1 | Inverse complement | 41 | 599 | GCGTTCGTCTATTACTCAATCAATCCGTGTGTAGTA |
| 10 | 1 | Inverse complement | 42 | 600 | TCATAGTGCGAGGATCCGAACATTGTCTAACCGATC |
| 10 | 1 | Inverse complement | 43 | 601 | GGTATGTTAAGCAGTTAGATACACTGTGACAGGTGG |
| 10 | 1 | Inverse complement | 44 | 602 | TGTACTGCCCAACGAGTTGTATTCTGCAATGAGTCG |
| 10 | 1 | Inverse complement | 45 | 603 | GCCGCCAATTATTTGCATTGCTGTACTCACAGGGAC |
| 10 | 1 | Inverse complement | 46 | 604 | AAAGTTCATAAAGGCTGTAAGGACGGATCAAGGCGC |
| 10 | 1 | Inverse complement | 47 | 605 | AAGCATTGATCTGATCGCATGCAGCGATATCCAGAA |
| 10 | 1 | Inverse complement | 48 | 606 | TCACCTTAAGCCACGGCGCATATTTAGTTGACTGAC |
| 10 | 1 | Inverse complement | 49 | 607 | AAACGGAGTAGGAATGTGTTCTGTGGAAGAGGATGT |
| 10 | 1 | Inverse complement | 50 | 608 | CTATAGTTGTTGGGTGGGACGGAATAGAAGCACATA |
| 10 | 1 | Inverse complement | 51 | 609 | GGTCTATATCAATTTAATCGACGCGCACATGTATGC |
| 10 | 1 | Inverse complement | 52 | 610 | ACTTATAATGGGCAGGGCGCTGGAAGTTACTCAGCA |
| 10 | 1 | Inverse complement | 53 | 611 | AGTAGTTAACCCTGTACAATAAGGAGGCATTCGAAC |
| 10 | 1 | Inverse complement | 54 | 612 | AATATCTTGTTTCAGTTCCCGGCACGCATTAAGCCC |
| 10 | 1 | Inverse complement | 55 | 613 | CGGAGTCATCCGGGATTCAATTGTAGTCGATACATG |
| 10 | 1 | Inverse complement | 56 | 614 | TCGTCCGGTTACCTCACCATTAAGTAGAACCGGAGT |
| 10 | 1 | Inverse complement | 57 | 615 | ATTCACAGCCGAGTATAGATTTCGTCTGCCTGTATT |
| 10 | 1 | Inverse complement | 58 | 616 | GTAACTATACTACTAGGAGCTCGGCCAGCTTGAATG |
| 10 | 1 | Inverse complement | 59 | 617 | CCTTCATGACCGCAGAAAGCTGCGAATAGTTTGATG |
| 10 | 1 | Inverse complement | 60 | 618 | TTATATGCTCTTTGGGTATTCTATCGCGCGTAAGCA |
| 10 | 1 | Inverse complement | 61 | 619 | TTGTCGATGATGGACCCAGAATTACGCCTCCAACCT |
| 10 | 1 | Inverse complement | 62 | 620 | GGGTGACTAGATTGTACTCCGGCCACCCTAAACTTT |
| 10 | 1 | Inverse complement | 63 | 621 | GGCTGGAGCATGATTGGCGACGGGACTATTTCTTAA |
| 10 | 1 | Inverse complement | 64 | 622 | GCAAGCACTGTTTCTAGGCCCGGAGAGATAGTATCA |

TABLE 11

Capture Oligonucleotide set 11: 36-mer non-cross-reactive capture oligonucleotides having sequences that are the inverse complement of the sequences generated using base oligonucleotide #2

| Run | Base | Transform | Parent SEQ ID NO | SEQ ID NO | Sequence |
|---|---|---|---|---|---|
| 11 | 2 | Inverse complement | 65 | 623 | CAGCAGCAAGTCTCATGCATAGTTTCCTTACTAGTT |
| 11 | 2 | Inverse complement | 66 | 624 | TTGTCGTGCCGGAGGTCTAGGAGACAGAAACGTATT |
| 11 | 2 | Inverse complement | 67 | 625 | AATGACTTTGCGACTCAGAAAGGCGAATCGATGACC |
| 11 | 2 | Inverse complement | 68 | 626 | TAAGAAGCTTGCTCGGTAAGTTAACCATATGCCTCA |
| 11 | 2 | Inverse complement | 69 | 627 | AATCTCTCGCGCACGTGATAAACATCCATCCATACG |
| 11 | 2 | Inverse complement | 70 | 628 | TAGACCAGAACTCTAATTTATCGAAACGGCCAACAC |
| 11 | 2 | Inverse complement | 71 | 629 | ATCGAATTAGACAAGCGTGTTTCATGACGAGCAGGG |
| 11 | 2 | Inverse complement | 72 | 630 | GGCGTTAAAGAGTTAATGCTGATTTCGAGAGCGCCG |
| 11 | 2 | Inverse complement | 73 | 631 | CTGGCGTGATATAAAGTCGAGAGTAACCCGTAACGT |
| 11 | 2 | Inverse complement | 74 | 632 | TCGCCGAACAAATCTGAGATAAGGACTCCTGGACTG |
| 11 | 2 | Inverse complement | 75 | 633 | GTATGGTTGCTGTCAGGTTAGTGAAAGGAAGTGAGG |
| 11 | 2 | Inverse complement | 76 | 634 | ATATAGCTTTAGTGGCGTTGCTGAGCGAAAGAGTAA |
| 11 | 2 | Inverse complement | 77 | 635 | TACAAATTTCTTTAGGGCCTGCCGATACAGTTCGCC |
| 11 | 2 | Inverse complement | 78 | 636 | GTCGATATGATAGTACACAACTAGCCTCTGGCGACT |
| 11 | 2 | Inverse complement | 79 | 637 | TTATTGTCGCACCATATAGTAGGCTGCACAGATAGA |
| 11 | 2 | Inverse complement | 80 | 638 | CATACATCTTCCAGTTGACGTCGCTCGCTTAAGGAA |
| 11 | 2 | Inverse complement | 81 | 639 | AGCGATAAACTGAACATATCCAGGGAGGTGATCCAT |
| 11 | 2 | Inverse complement | 82 | 640 | TTTCACCCAACCATGGGCTAAGAATAGTTTGAGATG |
| 11 | 2 | Inverse complement | 83 | 641 | CTGCGTAATCAACAGATCTTGGTAGTAGTTGGACAG |
| 11 | 2 | Inverse complement | 84 | 642 | TGATAATTGTGGTAGTCCCAGTTACGGTACCTTCCA |
| 11 | 2 | Inverse complement | 85 | 643 | ATGCTCAATCCAGGTACACTTGCGTATCCGACGCTT |
| 11 | 2 | Inverse complement | 86 | 644 | ATCAGTTCCAGAAAGTGGACAACGGCGACATATATA |
| 11 | 2 | Inverse complement | 87 | 645 | TATAATAATCGTGCGGGCTCATAACGAATCCGGCTG |
| 11 | 2 | Inverse complement | 88 | 646 | TACTTAGATGCACCGCAACACAGCCCATAGCAGCTA |
| 11 | 2 | Inverse complement | 89 | 647 | TCAATGAGTGGCACCTGCTTATTTGTAACGATCGGG |
| 11 | 2 | Inverse complement | 90 | 648 | GGGAGCGCTTTGAACTTCGATGTCTGTACACGTACT |
| 11 | 2 | Inverse complement | 91 | 649 | CTGACTAAGGAGATGAATGACAGACCTACCTGTCCA |
| 11 | 2 | Inverse complement | 92 | 650 | TGTTAGTCCGTCCTACTGTGGTTAGACCGATCTGAG |
| 11 | 2 | Inverse complement | 93 | 651 | ACCTCGCATTCGGAACCTCGGAACTAGATCTTACTT |
| 11 | 2 | Inverse complement | 94 | 652 | GCTTCGCAGAATTGGAATCTTGAAAGGTCACTCACC |
| 11 | 2 | Inverse complement | 95 | 653 | GCTTGTAAGTCCCAAGACCAAGCAGTCAGCGGATTT |
| 11 | 2 | Inverse complement | 96 | 654 | AGGAGTTAGCGAGATGTAGGCTCCTCGTTCTATGTT |
| 11 | 2 | Inverse complement | 97 | 655 | ACACAACCCTGCCATTACTCCTGTGTTAGCTAGTCA |
| 11 | 2 | Inverse complement | 98 | 656 | TCGAATGAGTTTGTTCAATCGTGACTTCCGATGTGT |
| 11 | 2 | Inverse complement | 99 | 657 | AGCTTAACGTCGTGGCTGGAACACTCAGGATTTGTA |
| 11 | 2 | Inverse complement | 100 | 658 | TCGCTTCACTCTCTCTTACCACTAGGCACACTAATA |
| 11 | 2 | Inverse complement | 101 | 659 | ACCTGGCCTTCCCAATTAGCATACGCCGTACTTGTT |
| 11 | 2 | Inverse complement | 102 | 660 | TTATCCCTGAGATTGAGTTAGAGCACTGTCGTATGT |
| 11 | 2 | Inverse complement | 103 | 661 | TCCGAGCAGTTTACCAGTTTGGAAGTGTTTGTTGAG |
| 11 | 2 | Inverse complement | 104 | 662 | CCTGGCAAGATGAGCTGCTTGAATGTGAACCAGCAT |
| 11 | 2 | Inverse complement | 105 | 663 | ACGCTGTCAACGATGTACAGGAGAATGGTGGGAAGT |
| 11 | 2 | Inverse complement | 106 | 664 | CGGCACTTAGCACGAAACCTACGAGTTATATGTGAT |
| 11 | 2 | Inverse complement | 107 | 665 | GATAGACGATGAGTCTCTTTGCTCCAAGTCCTAGAG |
| 11 | 2 | Inverse complement | 108 | 666 | CTAGGACCCTAATAACTACACCGTGGGTTTGCGTTC |
| 11 | 2 | Inverse complement | 109 | 667 | CGCCTTGGTGTCGAAAGAATCCAACGTAGTTAAATC |
| 11 | 2 | Inverse complement | 110 | 668 | TAGACACGCCATCTACGTGCAGTAATCAGATGATAT |
| 11 | 2 | Inverse complement | 111 | 669 | GGCTGCAATAAGCTAAGCCCTTTGTCTGACACGAAG |
| 11 | 2 | Inverse complement | 112 | 670 | AGACCGCGACCCATCATTGCATTGTGTCAAATTAAT |
| 11 | 2 | Inverse complement | 113 | 671 | TCACCCGCGTCTTGTTGGCGTCAATTCTATTGTGAT |
| 11 | 2 | Inverse complement | 114 | 672 | GCATGCATCCCTGTGGCTGTACTCTATATTGTATCT |
| 11 | 2 | Inverse complement | 115 | 673 | GTAAAGCGGCAGAAGCATTGTTGTTGCCGCAAAGCA |
| 11 | 2 | Inverse complement | 116 | 674 | TGCTCACTGCCTCACTGACCCAAACCTTAATACGAA |
| 11 | 2 | Inverse complement | 117 | 675 | CATGAAGTCGCGCCACGCACATCGTACTCTTAATAG |
| 11 | 2 | Inverse complement | 118 | 676 | ACCGAGAACTTGTGCATTGCTTGATAGTTACGCCAA |
| 11 | 2 | Inverse complement | 119 | 677 | AATTTGTTATTCTAGGGTCTTGCCTCGGACCGGGTT |
| 11 | 2 | Inverse complement | 120 | 678 | TACTTGTGAGAGGTAGAACGTGATTCAGTCCTTCGT |
| 11 | 2 | Inverse complement | 121 | 679 | GGGAGTCTAAGACACTAAGGTGAAATATCCTGTGCC |
| 11 | 2 | Inverse complement | 122 | 680 | CTGTAGTATATGCACTTGTCGTCCTCTTGGTTATGC |

TABLE 12

Capture Oligonucleotide set 12: 36-mer non-cross-reactive capture oligonucleotides having sequences that are the inverse complement of the sequences generated using base oligonucleotide #3

| Run | Base | Transform | Parent SEQ ID NO | SEQ ID NO | Sequence |
|---|---|---|---|---|---|
| 12 | 3 | Inverse complement | 123 | 681 | CGATGACACCTCTCCCAAACACTTAGATCCTCGTGT |
| 12 | 3 | Inverse complement | 124 | 682 | TGTCGTTACGATCCCTCGTTATTTGTATTGGTAGGT |
| 12 | 3 | Inverse complement | 125 | 683 | CTCTGCAAGAGAAAGGTAAACCTAGTAAAGCCTGGC |
| 12 | 3 | Inverse complement | 126 | 684 | TTGGTCTTCAACACCGGGTATGAGCAAATGACCCAC |
| 12 | 3 | Inverse complement | 127 | 685 | CGCTTAGCATTGAGGTGGTGTCTCATGCTGCTAAAC |
| 12 | 3 | Inverse complement | 128 | 686 | GCCAGCATCGGGAATATAAGCGAGTCATGCTAACTG |
| 12 | 3 | Inverse complement | 129 | 687 | CCAAGGCGAACGCTGGCAAATCTACAAAGTCTTGTC |
| 12 | 3 | Inverse complement | 130 | 688 | CCGACAAGCGCACTATGCAGCATTTGGATCTATCAG |
| 12 | 3 | Inverse complement | 131 | 689 | TTGGTACAGATCATAAACAGTGCAGGACATACTGGC |
| 12 | 3 | Inverse complement | 132 | 690 | ACATGAAGCGGTGTGGACAGGAACACCAAACGGATT |
| 12 | 3 | Inverse complement | 133 | 691 | TGATCCCAGGAAGTCTCGGCCATCTACATACCGTAT |
| 12 | 3 | Inverse complement | 134 | 692 | AAATGTGACCATATCCCTGCCACACATCGGCTCGAT |
| 12 | 3 | Inverse complement | 135 | 693 | CACCCAATTTATACTAAACCTCCTGCTCTGCGCGTA |
| 12 | 3 | Inverse complement | 136 | 694 | TTTCAAGACTGCCGATAAGCGTCAAAGGTGCCTTGA |
| 12 | 3 | Inverse complement | 137 | 695 | TCTAACGGAAGCACACGTCTTATCGCCGTAGCAGAA |
| 12 | 3 | Inverse complement | 138 | 696 | TAAGGTTCCGCTATGCCCGAGCAGAATTGCCCTTAA |
| 12 | 3 | Inverse complement | 139 | 697 | ATATTCGGAGCGGACTGTCTGCAATAACAGATGTGT |
| 12 | 3 | Inverse complement | 140 | 698 | ACGACCGAATTGCGGGTAGATGTGAATCGATATCTT |
| 12 | 3 | Inverse complement | 141 | 699 | GCCCTCCAATATTTGGCGTGTATCATTTATCGAGTT |
| 12 | 3 | Inverse complement | 142 | 700 | TAGTCAAGCGGACACTCGTCGTCGATTATGGACATT |
| 12 | 3 | Inverse complement | 143 | 701 | TTTCCATAGTTGATGCATATGGACCCTACTTGTCTG |
| 12 | 3 | Inverse complement | 144 | 702 | GAGGTCCCTATCAAAGATACTCAAACTTGTTGCAGC |
| 12 | 3 | Inverse complement | 145 | 703 | GAGATAAACTGCTTGACAGACATTCGTGGGTTCCTA |
| 12 | 3 | Inverse complement | 146 | 704 | CTCAATATACTTCCTTTCACAGAGCCGGCATGAAAG |
| 12 | 3 | Inverse complement | 147 | 705 | CAGACCAGCACATGGGTGTTTATATCCCGACAGAAC |
| 12 | 3 | Inverse complement | 148 | 706 | TTGTAGGTCTATCGCTTTGGTCAGAAATGAAACCGG |
| 12 | 3 | Inverse complement | 149 | 707 | TGATTTCGCGAGCTAGGTGGTAAAGAACTTGACGTT |
| 12 | 3 | Inverse complement | 150 | 708 | CCTGTACATCAGATTGTGACCCGCAGTATCCTAACG |
| 12 | 3 | Inverse complement | 151 | 709 | TAGAAGCTTGAGCGAAGTTGGACCTGACACGACAAT |
| 12 | 3 | Inverse complement | 152 | 710 | AGGGCACGAGTTAACGCTAATGATGTTTCTCATCGG |
| 12 | 3 | Inverse complement | 153 | 711 | AGTTAAAGAGCGGCCTCAAACGGTGACGAAGGATAC |
| 12 | 3 | Inverse complement | 154 | 712 | TAGTGATATGATACCTGCGTACCTCTCACCCATAGG |
| 12 | 3 | Inverse complement | 155 | 713 | GTGCCAAACTAATCTACGTTATCGCAACCGACTTAC |
| 12 | 3 | Inverse complement | 156 | 714 | GATGAGAGACTTATGTAATAGGCTCACCCGCTCCAA |
| 12 | 3 | Inverse complement | 157 | 715 | GGCGACCATTCAACTAACACGTTGGGCATTGGAAAT |
| 12 | 3 | Inverse complement | 158 | 716 | TCCTTATTTCGCTGTATTCTTCTCGTACGTGAGAAC |
| 12 | 3 | Inverse complement | 159 | 717 | GACTGAGGATTCACTACTCTTGTATATCGGGTGTCC |
| 12 | 3 | Inverse complement | 160 | 718 | TTAGCAAGCAATCACGATGCGGAAGTGAATTCGATA |
| 12 | 3 | Inverse complement | 161 | 719 | GTTGGACATAGCCTCTGGTAAACATTAGTGTAGCGG |
| 12 | 3 | Inverse complement | 162 | 720 | TAGTTGCAAACGTTATTCTAAGTCGACCTCATCTCG |
| 12 | 3 | Inverse complement | 163 | 721 | TGATACGAGGCCATTACCCAGTAATCTAAGCTTCCT |
| 12 | 3 | Inverse complement | 164 | 722 | GATGTAGCTGCTTACGAAACAGGTGATAATTGCAGC |
| 12 | 3 | Inverse complement | 165 | 723 | AATAGCACCACACTATTGACTAAACGAAAGCCCTCA |
| 12 | 3 | Inverse complement | 166 | 724 | CCTAGATGTCACTGAGATAGCCCAACCCATCAAGAA |
| 12 | 3 | Inverse complement | 167 | 725 | TAAGGACTGGCCTACCGACATCCTGTATCAACATTC |
| 12 | 3 | Inverse complement | 168 | 726 | TACGACTGCGACTCCATGCGATTGTCCTGTTTAGGT |
| 12 | 3 | Inverse complement | 169 | 727 | TGTTAATCGCCGGTATAGACAATTCAGTAAGGAGGC |
| 12 | 3 | Inverse complement | 170 | 728 | ACGTATTTCTTAGGAGCCTCATCAACCTAGGACTAC |
| 12 | 3 | Inverse complement | 171 | 729 | CCTGTCCGGTCAATTTGTAACGCCCTTCGAATTGAT |
| 12 | 3 | Inverse complement | 172 | 730 | AAACGCGGGCCACCAATTAGGGATTATCTAGAGTGA |
| 12 | 3 | Inverse complement | 173 | 731 | CCAACCACAGACGTTTAACGACCGCAACCATTAGAC |
| 12 | 3 | Inverse complement | 174 | 732 | TGCGACATAGAGGCCGACAGTTATACACTTAAGGCG |
| 12 | 3 | Inverse complement | 175 | 733 | AGGTGAAATCAGACGTCAGCCACGAGAGAATGAGAT |
| 12 | 3 | Inverse complement | 176 | 734 | TCATTAATTCCGAAGGGTAACTAGGCGGCTCGTAAT |
| 12 | 3 | Inverse complement | 177 | 735 | GTCTTATATGGAAGCCATCGTGTCGTCTGGAATCCA |
| 12 | 3 | Inverse complement | 178 | 736 | GGCTTTGACAACTAGTAGCGCGAAAGTTTGCCCAAT |
| 12 | 3 | Inverse complement | 179 | 737 | TACTGAGCCGCTAGAACAGACCCTCGATCGTTTAAG |
| 12 | 3 | Inverse complement | 180 | 738 | CCCAGTGATGCAATTTCACAACCATACCGGGAGATT |
| 12 | 3 | Inverse complement | 181 | 739 | AAGTTGTGGCAATACCTAGGCACGGCTTAGTCTAGC |
| 12 | 3 | Inverse complement | 182 | 740 | AGTCAGACATACCTCATAGCCGTGAAGGTGTAGGAC |
| 12 | 3 | Inverse complement | 183 | 741 | ACGCCCGTTATCATCGAACCTAGAGCACGTTAATCC |
| 12 | 3 | Inverse complement | 184 | 742 | TTAAGGCCAAATGGCAGCGAGTGTAAAGGACCTCTG |
| 12 | 3 | Inverse complement | 185 | 743 | TCAACACAACACGCTTTAATCCGCCTACGATGCCCT |
| 12 | 3 | Inverse complement | 186 | 744 | TGCAACGGACCGACTCACACAATTACTACAGAGCTA |

### T. Oligonucleotide Tags

TABLE 13

Tag set 1: 24-mer non-cross-reactive oligonucleotide tags that hybridize to the capture sequences generated using base oligonucleotide #1

| Run | Base | Transform | Parent SEQ ID NO | SEQ ID NO | Sequence |
|---|---|---|---|---|---|
| 1 | 1 | None | 1 | 745 | ACTGGTAACCCAGACATGATCGGT |
| 1 | 1 | None | 2 | 746 | CATTTGGTCATTGGTTCAAGACGA |
| 1 | 1 | None | 3 | 747 | CTAATAGCTCCTGTGCCCTCGTAT |
| 1 | 1 | None | 4 | 748 | GTGGCAACAGAAATCAGGTGGTGA |
| 1 | 1 | None | 5 | 749 | TAGATGCCGCTGCAGGTATGGAAA |
| 1 | 1 | None | 6 | 750 | ACTCCCTGTGGGTGAGCTTAATGG |
| 1 | 1 | None | 7 | 751 | CTGGTCCGTTGTGGTCCTTCTAAC |
| 1 | 1 | None | 8 | 752 | AATCCGTCGACTAGCCTGAGAATT |
| 1 | 1 | None | 9 | 753 | AGAGGACTGCTAAAGGTTTGTAGG |
| 1 | 1 | None | 10 | 754 | CGTACCATTGAATCTGGAGACCTT |
| 1 | 1 | None | 11 | 755 | ATTCCTCACAGAGAACAGACAGTT |
| 1 | 1 | None | 12 | 756 | CATTAAACGACAATGGTTACGTGA |
| 1 | 1 | None | 13 | 757 | TCGGGATAGCATCATCTGGCATAG |
| 1 | 1 | None | 14 | 758 | GAATAGGTAGTTGTGACGTAGGGA |
| 1 | 1 | None | 15 | 759 | CCCGCTTGGTTAGCTAGATAGCAC |
| 1 | 1 | None | 16 | 760 | CACTATGGCAGTAACCCTTAGTAT |
| 1 | 1 | None | 17 | 761 | CTTGGTGTCTAGAAAGAAACACCT |
| 1 | 1 | None | 18 | 762 | GAAAGCAAACGTTGCTCCGTCTCG |
| 1 | 1 | None | 19 | 763 | AAACACTTTATGTAAGATTCCGTC |
| 1 | 1 | None | 20 | 764 | TACCCGACTTAAATCACGGTACTG |
| 1 | 1 | None | 21 | 765 | CCAAATCCGGTATAACGTAGAGAC |
| 1 | 1 | None | 22 | 766 | GCTTTCCCGCAGTCTCAAAGAAAG |
| 1 | 1 | None | 23 | 767 | CACTGAGCCAAGCCGTGTAAGTAC |
| 1 | 1 | None | 24 | 768 | GTTATTTACCTACAGAACACCGAG |
| 1 | 1 | None | 25 | 769 | GGCCAGGTTCGCTTGGGATGTTAC |
| 1 | 1 | None | 26 | 770 | TTCGCGACTGTTGCAGCACTACCC |
| 1 | 1 | None | 27 | 771 | TATGTGTGTGATAAATGACTGACT |
| 1 | 1 | None | 28 | 772 | CGAAGGGTGATGAGCATCTTTGAA |
| 1 | 1 | None | 29 | 773 | AGGGACAGGCGCACTCGAGACTAT |
| 1 | 1 | None | 30 | 774 | GTAATACAGGTAGATCGCGTAGGA |
| 1 | 1 | None | 31 | 775 | TGTGACGGATCAGCTTCCGCAGAA |
| 1 | 1 | None | 32 | 776 | GGTTGTATTCAAGCGGCGGAACCA |
| 1 | 1 | None | 33 | 777 | AGCGTACTGCTCGTAAACGCTCAC |
| 1 | 1 | None | 34 | 778 | TGGCGTTCCAACAATAACACCCTG |
| 1 | 1 | None | 35 | 779 | CGATGTTGAAAGTTCATTTGAGCG |
| 1 | 1 | None | 36 | 780 | CTAAGAAGCATCTGCCCAATACCA |
| 1 | 1 | None | 37 | 781 | CGTAAATTTCGTGCATGGCAAGAT |
| 1 | 1 | None | 38 | 782 | GCGGGTTCCTGTTTAAGTAGTATA |
| 1 | 1 | None | 39 | 783 | GCATTTCTGAATGTAGGGATCTAC |
| 1 | 1 | None | 40 | 784 | ATTATATTCGCGCACTTAGCACAC |
| 1 | 1 | None | 41 | 785 | TAACTAACTCATTATCTGCTTGCG |
| 1 | 1 | None | 42 | 786 | TTACAAGCCTAGGAGCGTGATACT |
| 1 | 1 | None | 43 | 787 | CACATAGATTGACGAATTGTATGG |
| 1 | 1 | None | 44 | 788 | CTTATGTTGAGCAACCCGTCATGT |
| 1 | 1 | None | 45 | 789 | TGTCGTTACGTTTATTAACCGCCG |
| 1 | 1 | None | 46 | 790 | CAGGAATGTCGGAAATACTTGAAA |
| 1 | 1 | None | 47 | 791 | GACGTACGCTAGTCTAGTTACGAA |
| 1 | 1 | None | 48 | 792 | TTATACGCGGCACCGAATTCCACT |
| 1 | 1 | None | 49 | 793 | TGTCTTGTGTAAGGATGAGGCAAA |
| 1 | 1 | None | 50 | 794 | AAGGCAGGGTGGGTTGTTGATATC |
| 1 | 1 | None | 51 | 795 | CGCAGCTAATTTAACTATATCTGG |
| 1 | 1 | None | 52 | 796 | AGGTCGCGGGACGGGTAATATTCA |
| 1 | 1 | None | 53 | 797 | GGAATAACATGTCCCAATTGATGA |
| 1 | 1 | None | 54 | 798 | ACGGCCCTTGACTTTGTTCTATAA |
| 1 | 1 | None | 55 | 799 | TGTTAACTTAGGGCCTACTGAGGC |
| 1 | 1 | None | 56 | 800 | GAATTACCACTCCATTGGCCTGCT |
| 1 | 1 | None | 57 | 801 | GCTTTAGATATGAGCCGACACTTA |
| 1 | 1 | None | 58 | 802 | GGCTCGAGGATCATCATATCAATG |
| 1 | 1 | None | 59 | 803 | GCGTCGAAAGACGCCAGTACTTCC |
| 1 | 1 | None | 60 | 804 | TATCTTATGGGTTTCTCGTATATT |
| 1 | 1 | None | 61 | 805 | ATTAAGACCCAGGTAGTAGCTGTT |
| 1 | 1 | None | 62 | 806 | CCGGCCTCATGTTAGATCAGTGGG |
| 1 | 1 | None | 63 | 807 | GGGCAGCGGTTAGTACGAGGTCGG |
| 1 | 1 | None | 64 | 808 | AGGCCCGGATCTTTGTCACGAACG |

TABLE 14

Tag set 2: 24-mer non-cross-reactive oligonucleotide tags that hybridize to the capture oligonucleotides generated using base oligonucleotide #2

| Run | Base | Transform | Parent SEQ ID NO | SEQ ID NO | Sequence |
|---|---|---|---|---|---|
| 2 | 2 | None | 65 | 809 | TTGATACGTACTCTGAACGACGAC |
| 2 | 2 | None | 66 | 810 | AGAGGATCTGGAGGCCGTGCTGTT |
| 2 | 2 | None | 67 | 811 | CGGAAAGACTCAGCGTTTCAGTAA |
| 2 | 2 | None | 68 | 812 | AATTGAATGGCTCGTTCGAAGAAT |
| 2 | 2 | None | 69 | 813 | ACAAATAGTGCACGCGCTCTCTAA |
| 2 | 2 | None | 70 | 814 | AGCTATTTAATCTCAAGACCAGAT |
| 2 | 2 | None | 71 | 815 | ACTTTGTGCGAACAGATTAAGCTA |
| 2 | 2 | None | 72 | 816 | TTAGTCGTAATTGAGAAATTGCGG |
| 2 | 2 | None | 73 | 817 | TGAGAGCTGAAATATAGTGCGGTC |
| 2 | 2 | None | 74 | 818 | GGAATAGAGTCTAAACAAGCCGCT |
| 2 | 2 | None | 75 | 819 | AGTGATTGGACTGTCGTTGGTATG |
| 2 | 2 | None | 76 | 820 | AGTCGTTGCGGTGATTTCGATATA |
| 2 | 2 | None | 77 | 821 | GCCGTCCGGGATTTCTTTAAACAT |
| 2 | 2 | None | 78 | 822 | GATCAACACATGATAGTATAGCTG |
| 2 | 2 | None | 79 | 823 | CGGATGATATACCACGCTGTTATT |
| 2 | 2 | None | 80 | 824 | CGCTGCAGTTGACCTTCTACATAC |
| 2 | 2 | None | 81 | 825 | GGACCTATACAAGTCAAATAGCGA |
| 2 | 2 | None | 82 | 826 | AAGAATCGGGTACCAACCCACTTT |
| 2 | 2 | None | 83 | 827 | ATGGTTCTAGACAACTAATGCGTC |
| 2 | 2 | None | 84 | 828 | ATTGACCCTGATGGTGTTAATAGT |
| 2 | 2 | None | 85 | 829 | GCGTTCACATGGACCTAACTCGTA |
| 2 | 2 | None | 86 | 830 | GCAACAGGTGAAAGACCTTGACTA |
| 2 | 2 | None | 87 | 831 | AATACTCGGGCGTGCTAATAATAT |
| 2 | 2 | None | 88 | 832 | CGACACAACGCCACGTAGATTCAT |
| 2 | 2 | None | 89 | 833 | TTTATTCGTCCACGGTGAGTAACT |
| 2 | 2 | None | 90 | 834 | CTGTAGCTTCAAGTTTCGCGAGGG |
| 2 | 2 | None | 91 | 835 | AGACAGTAAGTAGAGGAATCAGTC |
| 2 | 2 | None | 92 | 836 | ATTGGTGTCATCCTGCCTGATTGT |
| 2 | 2 | None | 93 | 837 | CAAGGCTCCAAGGCTTACGCTCCA |
| 2 | 2 | None | 94 | 838 | AAGTTCTAAGGTTAAGACGCTTCG |
| 2 | 2 | None | 95 | 839 | ACGAACCAGAACCCTGAATGTTCG |
| 2 | 2 | None | 96 | 840 | CCTCGGATGTAGAGCGATTGAGGA |
| 2 | 2 | None | 97 | 841 | TGTCCTCATTACCGTCCCAACACA |
| 2 | 2 | None | 98 | 842 | AGTGCTAACTTGTTTGAGTAAGCT |
| 2 | 2 | None | 99 | 843 | CACAAGGTCGGTGCTGCAATTCGA |
| 2 | 2 | None | 100 | 844 | ATCACCATTCTCTCTCACTTCGCT |
| 2 | 2 | None | 101 | 845 | CATACGATTAACCCTTCCGGTCCA |
| 2 | 2 | None | 102 | 846 | CGAGATTGAGTTAGAGTCCCTATT |
| 2 | 2 | None | 103 | 847 | AAGGTTTGACCATTTGACGAGCCT |
| 2 | 2 | None | 104 | 848 | TAAGTTCGTCGAGTAGAACGGTCC |
| 2 | 2 | None | 105 | 849 | AGAGGACATGTAGCAACTGTCGCA |
| 2 | 2 | None | 106 | 850 | AGCATCCAAAGCACGATTCACGGC |
| 2 | 2 | None | 107 | 851 | CTCGTTTCTCTGAGTAGCAGATAG |
| 2 | 2 | None | 108 | 852 | TGCCACATCAATAATCCCAGGATC |
| 2 | 2 | None | 109 | 853 | AACCTAAGAAAGCTGTGGTTCCGC |
| 2 | 2 | None | 110 | 854 | ATGACGTGCATCTACCGCACAGAT |
| 2 | 2 | None | 111 | 855 | GTTTCCCGAATCGAATAACGTCGG |
| 2 | 2 | None | 112 | 856 | GTTACGTTACTACCCAGCGCCAGA |
| 2 | 2 | None | 113 | 857 | AACTGCGGTTGTTCTGCGCCCACT |
| 2 | 2 | None | 114 | 858 | CTCATGTCGGTGTCCCTACGTACG |
| 2 | 2 | None | 115 | 859 | TGTTGTTACGAAGACGGCGAAATG |
| 2 | 2 | None | 116 | 860 | AAACCCAGTCACTCCGTCACTCGT |
| 2 | 2 | None | 117 | 861 | GCTACACGCACCGCGCTGAAGTAC |
| 2 | 2 | None | 118 | 862 | AGTTCGTTACGTGTTCAAGAGCCA |
| 2 | 2 | None | 119 | 863 | CCGTTCTGGGATCTTATTGTTTAA |
| 2 | 2 | None | 120 | 864 | TAGTGCAAGATGGAGAGTGTTCAT |
| 2 | 2 | None | 121 | 865 | AAGTGGAATCACAGAATCTGAGGG |
| 2 | 2 | None | 122 | 866 | CCTGCTGTTCACGTATATGATGTC |

TABLE 15

Tag set 3: 24-mer non-cross-reactive oligonucleotide tags that hybridize with the capture oligonucleotides generated using base oligonucleotide #3

| Run | Base | Transform | Parent SEQ ID NO | SEQ ID NO | Sequence |
|---|---|---|---|---|---|
| 3 | 3 | None | 123 | 867 | TTCACAAACCCTCTCCACAGTAGC |
| 3 | 3 | None | 124 | 868 | TTTATTGCTCCCTAGCATTGCTGT |
| 3 | 3 | None | 125 | 869 | ATCCAAATGGAAAGAGAACGTCTC |
| 3 | 3 | None | 126 | 870 | GAGTATGGGCCACAACTTCTGGTT |
| 3 | 3 | None | 127 | 871 | CTCTGTGGTGGAGTTACGATTCGC |
| 3 | 3 | None | 128 | 872 | GAGCGAATATAAGGGCTACGACCG |
| 3 | 3 | None | 129 | 873 | ATCTAAACGGTCGCAAGCGGAACC |
| 3 | 3 | None | 130 | 874 | TTACGACGTATCACGCGAACAGCC |
| 3 | 3 | None | 131 | 875 | ACGTGACAAATACTAGACATGGTT |
| 3 | 3 | None | 132 | 876 | CAAGGACAGGTGTGGCGAAGTACA |
| 3 | 3 | None | 133 | 877 | CTACCGGCTCTGAAGGACCCTAGT |
| 3 | 3 | None | 134 | 878 | ACACCGTCCCTATACCAGTGTAAA |
| 3 | 3 | None | 135 | 879 | TCCTCCAAATCATATTTAACCCAC |
| 3 | 3 | None | 136 | 880 | ACTGCGAATAGCCGTCAGAACTTT |
| 3 | 3 | None | 137 | 881 | CTATTCTGCACACGAAGGCAATCT |
| 3 | 3 | None | 138 | 882 | GACGAGCCCGTATCGCCTTGGAAT |
| 3 | 3 | None | 139 | 883 | AACGTCTGTCAGGCGAGGCTTATA |
| 3 | 3 | None | 140 | 884 | GTGTAGATGGGCGTTAAGCCAGCA |
| 3 | 3 | None | 141 | 885 | CTATGTGCGGTTTATAACCTCCCG |
| 3 | 3 | None | 142 | 886 | GCTGCTGCTCACAGGCGAACTGAT |
| 3 | 3 | None | 143 | 887 | CAGGTATACGTAGTTGATACCTTT |
| 3 | 3 | None | 144 | 888 | AACTCATAGAAACTATCCCTGGAG |
| 3 | 3 | None | 145 | 889 | TTACAGACAGTTCGTCAAATAGAG |
| 3 | 3 | None | 146 | 890 | GAGACACTTTCCTTCATATAACTC |
| 3 | 3 | None | 147 | 891 | ATATTTGTGGGTACACGACCAGAC |
| 3 | 3 | None | 148 | 892 | GACTGGTTTCGCTATCTGGATGTT |
| 3 | 3 | None | 149 | 893 | AAATGGTGGATCGAGCGCTTTAGT |
| 3 | 3 | None | 150 | 894 | CGCCCAGTGTTAGACTACATGTCC |
| 3 | 3 | None | 151 | 895 | CCAGGTTGAAGCGAGTTCGAAGAT |
| 3 | 3 | None | 152 | 896 | TAGTAATCGCAATTGAGCACGGGA |
| 3 | 3 | None | 153 | 897 | TGGCAAACTCCGGCGAGAAATTGA |
| 3 | 3 | None | 154 | 898 | TCCATGCGTCCATAGTATAGTGAT |
| 3 | 3 | None | 155 | 899 | GCTATTGCATCTAATCAAACCGTG |
| 3 | 3 | None | 156 | 900 | TCGGATAATGTATTCAGAGAGTAG |
| 3 | 3 | None | 157 | 901 | GTTGCACAATCAACTTACCAGCGG |
| 3 | 3 | None | 158 | 902 | CTCTTCTTATGTCGCTTTATTCCT |
| 3 | 3 | None | 159 | 903 | ATGTTCTCATCACTTAGGAGTCAG |
| 3 | 3 | None | 160 | 904 | AAGGCGTAGCACTAACGAACGATT |
| 3 | 3 | None | 161 | 905 | ACAAATGGTCTCCGATACAGGTTG |
| 3 | 3 | None | 162 | 906 | CTGAATCTTATTGCAAACGTTGAT |
| 3 | 3 | None | 163 | 907 | AATGACCCATTACCGGAGCATAGT |
| 3 | 3 | None | 164 | 908 | TGGACAAAGCATTCGTCGATGTAG |
| 3 | 3 | None | 165 | 909 | AAATCAGTTATCACACCACGATAA |
| 3 | 3 | None | 166 | 910 | ACCCGATAGAGTCACTGTAGATCC |
| 3 | 3 | None | 167 | 911 | TCCTACAGCCATCCGGTCAGGAAT |
| 3 | 3 | None | 168 | 912 | GTTAGCGTACCTCAGCGTCAGCAT |
| 3 | 3 | None | 169 | 913 | TTAACAGATATGGCCGCTAATTGT |
| 3 | 3 | None | 170 | 914 | ACTACTCCGAGGATTCTTTATGCA |
| 3 | 3 | None | 171 | 915 | CCGCAATGTTTAACTGGCCTGTCC |
| 3 | 3 | None | 172 | 916 | TAGGGATTAACCACCGGGCGCAAA |
| 3 | 3 | None | 173 | 917 | GCCAGCAATTTGCAGACACCAACC |
| 3 | 3 | None | 174 | 918 | TATTGACAGCCGGAGATACAGCGT |
| 3 | 3 | None | 175 | 919 | GCACCGACTGCAGACTAAAGTGGA |
| 3 | 3 | None | 176 | 920 | GATCAATGGGAAGCCTTAATTACT |
| 3 | 3 | None | 177 | 921 | CTGTGCTACCGAAGGTATATTCTG |
| 3 | 3 | None | 178 | 922 | AAGCGCGATGATCAACAGTTTCGG |
| 3 | 3 | None | 179 | 923 | TCCCAGACAAGATCGCCGAGTCAT |
| 3 | 3 | None | 180 | 924 | ACCAACACTTTAACGTAGTGACCC |
| 3 | 3 | None | 181 | 925 | GCACGGATCCATAACGGTGTTGAA |
| 3 | 3 | None | 182 | 926 | GTGCCGATACTCCATACAGACTGA |
| 3 | 3 | None | 183 | 927 | AGATCCAAGCTACTATTGCCCGCA |
| 3 | 3 | None | 184 | 928 | TGTGAGCGACGGTAAACCGGAATT |
| 3 | 3 | None | 185 | 929 | CGCCTAATTTCGCACAACACAACT |
| 3 | 3 | None | 186 | 930 | TTAACACACTCAGCCAGGCAACGT |

TABLE 16

Tag set 4: 24-mer non-cross-reactive oligonucleotide tags that hybridize with the complementary sequences of the sequences generated using base oligonucleotide #1

| Run | Base | Transform | Parent SEQ ID NO | SEQ ID NO | Sequence |
|---|---|---|---|---|---|
| 4 | 1 | Complement | 1 | 931 | TGGGTTACCAGTTAGTCGTGTCTC |
| 4 | 1 | Complement | 2 | 932 | AATGACCAAATGCAAGCCCTCCAT |
| 4 | 1 | Complement | 3 | 933 | AGGAGCTATTAGTGTAGCGAAAGG |
| 4 | 1 | Complement | 4 | 934 | TTCTGTTGCCACCGCATCAGTTTA |
| 4 | 1 | Complement | 5 | 935 | CAGCGGCATCTATTCATGACATGT |
| 4 | 1 | Complement | 6 | 936 | CCCACAGGGAGTTGGAGTCTAAAC |
| 4 | 1 | Complement | 7 | 937 | ACAACGGACCAGAGAGTGCATATA |
| 4 | 1 | Complement | 8 | 938 | AGTCGACGGATTTACCGTCACTCG |
| 4 | 1 | Complement | 9 | 939 | TAGCAGTCCTCTGTTGGTCTCTGC |
| 4 | 1 | Complement | 10 | 940 | TTCAATGGTACGACCATCCGACTC |
| 4 | 1 | Complement | 11 | 941 | TCTGTGAGGAATTCTCCTTCCGAG |
| 4 | 1 | Complement | 12 | 942 | TGTCGTTTAATGTCTCCCAGCGTT |
| 4 | 1 | Complement | 13 | 943 | ATGCTATCCCGACGATGAACGGTT |
| 4 | 1 | Complement | 14 | 944 | AACTACCTATTCGAGTGTGGTCTT |
| 4 | 1 | Complement | 15 | 945 | TAACCAAGCGGGCTCTTTATAGGC |
| 4 | 1 | Complement | 16 | 946 | ACTGCCATAGTGCCGACGCGTAGA |
| 4 | 1 | Complement | 17 | 947 | CTAGACACCAAGTATCCACAGGCA |
| 4 | 1 | Complement | 18 | 948 | ACGTTTGCTTTCAGTTAATCGGAG |
| 4 | 1 | Complement | 19 | 949 | CATAAAGTGTTTGGAGATGGTAGG |
| 4 | 1 | Complement | 20 | 950 | TTAAGTCGGGTAGACGATGCGGAT |
| 4 | 1 | Complement | 21 | 951 | TACCGGATTTGGGTATTCTCTGAG |
| 4 | 1 | Complement | 22 | 952 | CTGCGGGAAAGCGGTTCGGTAACT |
| 4 | 1 | Complement | 23 | 953 | CTTGGCTCAGTGCCCGTTTCATAT |
| 4 | 1 | Complement | 24 | 954 | TAGGTAAATAACGAGTAATCGCAC |
| 4 | 1 | Complement | 25 | 955 | GCGAACCTGGCCTTTAGTACCCAA |
| 4 | 1 | Complement | 26 | 956 | AACAGTCGCGAATTATAAATACGG |
| 4 | 1 | Complement | 27 | 957 | ATCACACACATACACAGTGAGCTC |
| 4 | 1 | Complement | 28 | 958 | CATCACCCTTCGCATCTCGGACCA |
| 4 | 1 | Complement | 29 | 959 | GCGCCTGTCCCTCTTACAGTTTCT |
| 4 | 1 | Complement | 30 | 960 | TACCTGTATTACTTTACTGGCAAC |
| 4 | 1 | Complement | 31 | 961 | TGATCCGTCACACAATCCTTCTTG |
| 4 | 1 | Complement | 32 | 962 | TTGAATACAACCAATACTTATCGG |
| 4 | 1 | Complement | 33 | 963 | GAGCAGTACGCTTCAACTCAATTC |
| 4 | 1 | Complement | 34 | 964 | GTTGGAACGCCAAATCGTCTGAAC |
| 4 | 1 | Complement | 35 | 965 | CTTTCAACATCGAATCCTTGCTGG |
| 4 | 1 | Complement | 36 | 966 | GATGCTTCTTAGGAATTGTGCAGT |
| 4 | 1 | Complement | 37 | 967 | ACGAAATTTACGATTAGGTCAGCG |
| 4 | 1 | Complement | 38 | 968 | ACAGGAACCCGCTCTCCGCAGGAC |
| 4 | 1 | Complement | 39 | 969 | ATTCAGAAATGCGTCTGTTGACGG |
| 4 | 1 | Complement | 40 | 970 | CGCGAATATAATAGCAAGTAGTTG |
| 4 | 1 | Complement | 41 | 971 | ATGAGTTAGTTAGGCACACATCAT |
| 4 | 1 | Complement | 42 | 972 | CTAGGCTTGTAACAGATTGGCTAG |
| 4 | 1 | Complement | 43 | 973 | TCAATCTATGTGACACTGTCCACC |
| 4 | 1 | Complement | 44 | 974 | GCTCAACATAAGACGTTACTCAGC |
| 4 | 1 | Complement | 45 | 975 | AACGTAACGACATGAGTGTCCCTG |
| 4 | 1 | Complement | 46 | 976 | CCGACATTCCTGCCTAGTTCCGCG |
| 4 | 1 | Complement | 47 | 977 | CTAGCGTACGTCGCTATAGGTCTT |
| 4 | 1 | Complement | 48 | 978 | TGCCGCGTATAAATCAACTGACTG |
| 4 | 1 | Complement | 49 | 979 | TTACACAAGACACCTTCTCCTACA |
| 4 | 1 | Complement | 50 | 980 | CCACCCTGCCTTATCTTCGTGTAT |
| 4 | 1 | Complement | 51 | 981 | AAATTAGCTGCGCGTGTACATACG |
| 4 | 1 | Complement | 52 | 982 | GTCCCGCGACCTTCAATGAGTCGT |
| 4 | 1 | Complement | 53 | 983 | ACATGTTATTCCTCCGTAAGCTTG |
| 4 | 1 | Complement | 54 | 984 | GTCAAGGGCCGTGCGTAATTCGGG |
| 4 | 1 | Complement | 55 | 985 | CCTAAGTTAACATCAGCTATGTAC |
| 4 | 1 | Complement | 56 | 986 | GAGTGGTAATTCATCTTGGCCTCA |
| 4 | 1 | Complement | 57 | 987 | CATATCTAAAGCAGACGGACATAA |
| 4 | 1 | Complement | 58 | 988 | GATCCTCGAGCCGGTCGAACTTAC |
| 4 | 1 | Complement | 59 | 989 | GTCTTTCGACGCTTATCAAACTAC |
| 4 | 1 | Complement | 60 | 990 | ACCCATAAGATAGCGCGCATTCGT |
| 4 | 1 | Complement | 61 | 991 | CTGGGTCTTAATGCGGAGGTTGGA |
| 4 | 1 | Complement | 62 | 992 | ACATGAGGCCGGTGGGATTTGAAA |
| 4 | 1 | Complement | 63 | 993 | TAACCGCTGCCCTGATAAAGAATT |
| 4 | 1 | Complement | 64 | 994 | AGATCCGGGCCTCTCTATCATAGT |

TABLE 17

Tag set 5: 24-mer non-cross-reactive oligonucleotide tags that hybridize with the complementary sequences of the sequences generated using base oligonucleotide #1

| Run | Base | Transform | Parent SEQ ID NO | SEQ ID NO | Sequence |
|---|---|---|---|---|---|
| 5 | 2 | Complement | 65 | 995 | AGTACGTATCAAAGGAATGATCAA |
| 5 | 2 | Complement | 66 | 996 | TCCAGATCCTCTGTCTTTGCATAA |
| 5 | 2 | Complement | 67 | 997 | TGAGTCTTTCCGCTTAGCTACTGG |
| 5 | 2 | Complement | 68 | 998 | AGCCATTCAATTGGTATACGGAGT |
| 5 | 2 | Complement | 69 | 999 | TGCACTATTTGTAGGTAGGTATGC |
| 5 | 2 | Complement | 70 | 1000 | GATTAAATAGCTTTGCCGGTTGTG |
| 5 | 2 | Complement | 71 | 1001 | TTCGCACAAAGTACTGCTCGTCCC |
| 5 | 2 | Complement | 72 | 1002 | AATTACGACTAAAGCTCTCGCGGC |
| 5 | 2 | Complement | 73 | 1003 | TTTCAGCTCTCATTGGGCATTGCA |
| 5 | 2 | Complement | 74 | 1004 | AGACTCTATTCCTGAGGACCTGAC |
| 5 | 2 | Complement | 75 | 1005 | AGTCCAATCACTTTCCTTCACTCC |
| 5 | 2 | Complement | 76 | 1006 | ACCGCAACGACTCGCTTTCTCATT |
| 5 | 2 | Complement | 77 | 1007 | ATCCCGGACGGCTATGTCAAGCGG |
| 5 | 2 | Complement | 78 | 1008 | CATGTGTTGATCGGAGACCGCTGA |
| 5 | 2 | Complement | 79 | 1009 | GTATATCATCCGACGTGTCTATCT |
| 5 | 2 | Complement | 80 | 1010 | TCAACTGCAGCGAGCGAATTCCTT |
| 5 | 2 | Complement | 81 | 1011 | TTGTATAGGTCCCTCCACTAGGTA |
| 5 | 2 | Complement | 82 | 1012 | TACCCGATTCTTATCAAACTCTAC |
| 5 | 2 | Complement | 83 | 1013 | GTCTAGAACCATCATCAACCTGTC |
| 5 | 2 | Complement | 84 | 1014 | ATCAGGGTCAATGCCATGGAAGGT |
| 5 | 2 | Complement | 85 | 1015 | CCATGTGAACGCATAGGCTGCGAA |
| 5 | 2 | Complement | 86 | 1016 | TTCACCTGTTGCCGCTGTATATAT |
| 5 | 2 | Complement | 87 | 1017 | CGCCCGAGTATTGCTTAGGCCGAC |
| 5 | 2 | Complement | 88 | 1018 | GGCGTTGTGTCGGGTATCGTCGAT |
| 5 | 2 | Complement | 89 | 1019 | TGGACGAATAAACATTGCTAGCCC |
| 5 | 2 | Complement | 90 | 1020 | TTGAAGCTACAGACATGTGCATGA |
| 5 | 2 | Complement | 91 | 1021 | TACTTACTGTCTGGATGGACAGGT |
| 5 | 2 | Complement | 92 | 1022 | GATGACACCAATCTGGCTAGACTC |
| 5 | 2 | Complement | 93 | 1023 | CTTGGAGCCTTGATCTAGAATGAA |
| 5 | 2 | Complement | 94 | 1024 | ACCTTAGAACTTTCCAGTGAGTGG |
| 5 | 2 | Complement | 95 | 1025 | GTTCTGGTTCGTCAGTCGCCTAAA |
| 5 | 2 | Complement | 96 | 1026 | CTACATCCGAGGAGCAAGATACAA |
| 5 | 2 | Complement | 97 | 1027 | GTAATGAGGACACAATCGATCAGT |
| 5 | 2 | Complement | 98 | 1028 | CAAGTTAGCACTGAAGGCTACACA |
| 5 | 2 | Complement | 99 | 1029 | ACCGACCTTGTGAGTCCTAAACAT |
| 5 | 2 | Complement | 100 | 1030 | GAGAATGGTGATCCGTGTGATTAT |
| 5 | 2 | Complement | 101 | 1031 | GTTAATCGTATGCGGCATGAACAA |
| 5 | 2 | Complement | 102 | 1032 | AACTCAATCTCGTGACAGCATACA |
| 5 | 2 | Complement | 103 | 1033 | TGGTCAAACCTTCACAAACAACTC |
| 5 | 2 | Complement | 104 | 1034 | TCGACGAACTTACACTTGGTCGTA |
| 5 | 2 | Complement | 105 | 1035 | TACATGTCCTCTTACCACCCTTCA |
| 5 | 2 | Complement | 106 | 1036 | GCTTTGGATGCTCAATATACACTA |
| 5 | 2 | Complement | 107 | 1037 | CAGAGAAACGAGGTTCAGGATCTC |
| 5 | 2 | Complement | 108 | 1038 | ATTGATGTGGCACCCAAACGCAAG |
| 5 | 2 | Complement | 109 | 1039 | CTTTCTTAGGTTGCATCAATTTAG |
| 5 | 2 | Complement | 110 | 1040 | GATGCACGTCATTAGTCTACTATA |
| 5 | 2 | Complement | 111 | 1041 | GATTCGGGAAACAGACTGTGCTTC |
| 5 | 2 | Complement | 112 | 1042 | TAGTAACGTAACACAGTTTAATTA |
| 5 | 2 | Complement | 113 | 1043 | ACAACCGCAGTTAAGATAACACTA |
| 5 | 2 | Complement | 114 | 1044 | CACCGACATGAGATATAACATAGA |
| 5 | 2 | Complement | 115 | 1045 | TTCGTAACAACAACGGCGTTTCGT |
| 5 | 2 | Complement | 116 | 1046 | GTGACTGGGTTTGGAATTATGCTT |
| 5 | 2 | Complement | 117 | 1047 | GGTGCGTGTAGCATGAGAATTATC |
| 5 | 2 | Complement | 118 | 1048 | ACGTAACGAACTATCAATGCGGTT |
| 5 | 2 | Complement | 119 | 1049 | ATCCCAGAACGGAGCCTGGCCCAA |
| 5 | 2 | Complement | 120 | 1050 | CATCTTGCACTAAGTCAGGAAGCA |
| 5 | 2 | Complement | 121 | 1051 | GTGATTCCACTTTATAGGACACGG |
| 5 | 2 | Complement | 122 | 1052 | GTGAACAGCAGGAGAACCAATACG |

TABLE 18

Tag set 6: 24-mer non-cross-reactive oligonucleotide tags that hybridize with the complementary sequences of the sequences generated using base oligonucleotide #3

| Run | Base | Transform | Parent SEQ ID NO | SEQ ID NO | Sequence |
|---|---|---|---|---|---|
| 6 | 3 | Complement | 123 | 1053 | AGGGTTTGTGAATCTAGGAGCACA |
| 6 | 3 | Complement | 124 | 1054 | GGGAGCAATAAACATAACCATCCA |
| 6 | 3 | Complement | 125 | 1055 | TTCCATTTGGATCATTTCGGACCG |
| 6 | 3 | Complement | 126 | 1056 | TGGCCCATACTCGTTTACTGGGTG |
| 6 | 3 | Complement | 127 | 1057 | TCCACCACAGAGTACGACGATTTG |
| 6 | 3 | Complement | 128 | 1058 | TTATATTCGCTCAGTACGATTGAC |
| 6 | 3 | Complement | 129 | 1059 | GACCGTTTAGATGTTTCAGAACAG |
| 6 | 3 | Complement | 130 | 1060 | GATACGTCGTAAACCTAGATAGTC |
| 6 | 3 | Complement | 131 | 1061 | TATTTGTCACGTCCTGTATGACCG |
| 6 | 3 | Complement | 132 | 1062 | CACCTGTCCTTGTGGTTTGCCTAA |
| 6 | 3 | Complement | 133 | 1063 | CAGAGCCGGTAGATGTATGGCATA |
| 6 | 3 | Complement | 134 | 1064 | TAGGGACGGTGTGTAGCCGAGCTA |
| 6 | 3 | Complement | 135 | 1065 | TGATTTGGAGGACGAGACGCGCAT |
| 6 | 3 | Complement | 136 | 1066 | GCTATTCGCAGTTTCCACGGAACT |
| 6 | 3 | Complement | 137 | 1067 | TGTGCAGAATAGCGGCATCGTCTT |
| 6 | 3 | Complement | 138 | 1068 | TACGGGCTCGTCTTAACGGGAATT |
| 6 | 3 | Complement | 139 | 1069 | CTGACAGACGTTATTGTCTACACA |
| 6 | 3 | Complement | 140 | 1070 | GCCCATCTACACTTAGCTATAGAA |
| 6 | 3 | Complement | 141 | 1071 | AACCGCACATAGTAAATAGCTCAA |
| 6 | 3 | Complement | 142 | 1072 | GTGAGCAGCAGCTAATACCTGTAA |
| 6 | 3 | Complement | 143 | 1073 | TACGTATACCTGGGATGAACAGAC |
| 6 | 3 | Complement | 144 | 1074 | TTTCTATGAGTTTGAACAACGTCG |
| 6 | 3 | Complement | 145 | 1075 | AACTGTCTGTAAGCACCCAAGGAT |
| 6 | 3 | Complement | 146 | 1076 | GGAAAGTGTCTCGGCCGTACTTTC |
| 6 | 3 | Complement | 147 | 1077 | ACCCACAAATATAGGGCTGTCTTG |
| 6 | 3 | Complement | 148 | 1078 | GCGAAACCAGTCTTTACTTTGGCC |
| 6 | 3 | Complement | 149 | 1079 | GATCCACCATTTCTTGAACTGCAA |
| 6 | 3 | Complement | 150 | 1080 | TAACACTGGGCGTCATAGGATTGC |
| 6 | 3 | Complement | 151 | 1081 | GCTTCAACCTGGACTGTGCTGTTA |
| 6 | 3 | Complement | 152 | 1082 | TTGCGATTACTACAAAGAGTAGCC |
| 6 | 3 | Complement | 153 | 1083 | CGGAGTTTGCCACTGCTTCCTATG |
| 6 | 3 | Complement | 154 | 1084 | TGGACGCATGGAGAGTGGGTATCC |
| 6 | 3 | Complement | 155 | 1085 | AGATGCAATAGCGTTGGCTGAATG |
| 6 | 3 | Complement | 156 | 1086 | TACATTATCCGAGTGGGCGAGGTT |
| 6 | 3 | Complement | 157 | 1087 | TGATTGTGCAACCCGTAACCTTTA |
| 6 | 3 | Complement | 158 | 1088 | ACATAAGAAGAGCATGCACTCTTG |
| 6 | 3 | Complement | 159 | 1089 | TGATGAGAACATATAGCCCACAGG |
| 6 | 3 | Complement | 160 | 1090 | GTGCTACGCCTTCACTTAAGCTAT |
| 6 | 3 | Complement | 161 | 1091 | GAGACCATTTGTAATCACATCGCC |
| 6 | 3 | Complement | 162 | 1092 | AATAAGATTCAGCTGGAGTAGAGC |
| 6 | 3 | Complement | 163 | 1093 | TAATGGGTCATTAGATTCGAAGGA |
| 6 | 3 | Complement | 164 | 1094 | ATGCTTTGTCCACTATTAACGTCG |
| 6 | 3 | Complement | 165 | 1095 | GATAACTGATTTGCTTTCGGGAGT |
| 6 | 3 | Complement | 166 | 1096 | ACTCTATCGGGTTGGGTAGTTCTT |
| 6 | 3 | Complement | 167 | 1097 | ATGGCTGTAGGACATAGTTGTAAG |
| 6 | 3 | Complement | 168 | 1098 | AGGTACGCTAACAGGACAAATCCA |
| 6 | 3 | Complement | 169 | 1099 | CATATCTGTTAAGTCATTCCTCCG |
| 6 | 3 | Complement | 170 | 1100 | CCTCGGAGTAGTTGGATCCTGATG |
| 6 | 3 | Complement | 171 | 1101 | TAAACATTGCGGGAAGCTTAACTA |
| 6 | 3 | Complement | 172 | 1102 | GGTTAATCCCTAATAGATCTCACT |
| 6 | 3 | Complement | 173 | 1103 | CAAATTGCTGGCGTTGGTAATCTG |
| 6 | 3 | Complement | 174 | 1104 | CGGCTGTCAATATGTGAATTCCGC |
| 6 | 3 | Complement | 175 | 1105 | TGCAGTCGGTGCTCTCTTACTCTA |
| 6 | 3 | Complement | 176 | 1106 | TTCCCATTGATCCGCCGAGCATTA |
| 6 | 3 | Complement | 177 | 1107 | TCGGTAGCACAGCAGACCTTAGGT |
| 6 | 3 | Complement | 178 | 1108 | ATCATCGCGCTTTCAAACGGGTTA |
| 6 | 3 | Complement | 179 | 1109 | TCTTGTCTGGGAGCTAGCAAATTC |
| 6 | 3 | Complement | 180 | 1110 | TAAAGTGTTGGTATGGCCCTCTAA |
| 6 | 3 | Complement | 181 | 1111 | ATGGATCCGTGCCGAATCAGATCG |
| 6 | 3 | Complement | 182 | 1112 | GAGTATCGGCACTTCCACATCCTG |
| 6 | 3 | Complement | 183 | 1113 | TAGCTTGGATCTCGTGCAATTAGG |
| 6 | 3 | Complement | 184 | 1114 | CCGTCGCTCACATTTCCTGGAGAC |
| 6 | 3 | Complement | 185 | 1115 | CGAAATTAGGCGGATGCTACGGGA |
| 6 | 3 | Complement | 186 | 1116 | TGAGTGTGTTAATGATGTCTCGAT |

TABLE 19

Tag set 7: 24-mer non-cross-reactive oligonucleotide tags that hybridize with the reverse of the sequences generated using base oligonucleotide #1

| Run | Base | Transform | Parent SEQ ID NO | SEQ ID NO | Sequence |
|---|---|---|---|---|---|
| 7 | 1 | Reverse | 1 | 1117 | ACCCAATGGTCAATCAGCACAGAG |
| 7 | 1 | Reverse | 2 | 1118 | TTACTGGTTTACGTTCGGGAGGTA |
| 7 | 1 | Reverse | 3 | 1119 | TCCTCGATAATCACATCGCTTTCC |
| 7 | 1 | Reverse | 4 | 1120 | AAGACAACGGTGGCGTAGTCAAAT |
| 7 | 1 | Reverse | 5 | 1121 | GTCGCCGTAGATAAGTACTGTACA |
| 7 | 1 | Reverse | 6 | 1122 | GGGTGTCCCTCAACCTCAGATTTG |
| 7 | 1 | Reverse | 7 | 1123 | TGTTGCCTGGTCTCTCACGTATAT |
| 7 | 1 | Reverse | 8 | 1124 | TCAGCTGCCTAAATGGCAGTGAGC |
| 7 | 1 | Reverse | 9 | 1125 | ATCGTCAGGAGACAACCAGAGACG |
| 7 | 1 | Reverse | 10 | 1126 | AAGTTACCATGCTGGTAGGCTGAG |
| 7 | 1 | Reverse | 11 | 1127 | AGACACTCCTTAAGAGGAAGGCTC |
| 7 | 1 | Reverse | 12 | 1128 | ACAGCAAATTACAGAGGGTCGCAA |
| 7 | 1 | Reverse | 13 | 1129 | TACGATAGGGCTGCTACTTGCCAA |
| 7 | 1 | Reverse | 14 | 1130 | TTGATGGATAAGCTCACACCAGAA |
| 7 | 1 | Reverse | 15 | 1131 | ATTGGTTCGCCCGAGAAATATCCG |
| 7 | 1 | Reverse | 16 | 1132 | TGACGGTATCACGGCTGCGCATCT |
| 7 | 1 | Reverse | 17 | 1133 | GATCTGTGGTTCATAGGTGTCCGT |
| 7 | 1 | Reverse | 18 | 1134 | TGCAAACGAAAGTCAATTAGCCTC |
| 7 | 1 | Reverse | 19 | 1135 | GTATTTCACAAACCTCTACCATCC |
| 7 | 1 | Reverse | 20 | 1136 | AATTCAGCCCATCTGCTACGCCTA |
| 7 | 1 | Reverse | 21 | 1137 | ATGGCCTAAACCCATAAGAGACTC |
| 7 | 1 | Reverse | 22 | 1138 | GACGCCCTTTCGCCAAGCCATTGA |
| 7 | 1 | Reverse | 23 | 1139 | GAACCGAGTCACGGGCAAAGTATA |
| 7 | 1 | Reverse | 24 | 1140 | ATCCATTTATTGCTCATTAGCGTG |
| 7 | 1 | Reverse | 25 | 1141 | CGCTTGGACCGGAAATCATGGGTT |
| 7 | 1 | Reverse | 26 | 1142 | TTGTCAGCGCTTAATATTTATGCC |
| 7 | 1 | Reverse | 27 | 1143 | TAGTGTGTGTATGTGTCACTCGAG |
| 7 | 1 | Reverse | 28 | 1144 | GTAGTGGGAAGCGTAGAGCCTGGT |
| 7 | 1 | Reverse | 29 | 1145 | CGCGGACAGGGAGAATGTCAAAGA |
| 7 | 1 | Reverse | 30 | 1146 | ATGGACATAATGAAATGACCGTTG |
| 7 | 1 | Reverse | 31 | 1147 | ACTAGGCAGTGTGTTAGGAAGAAC |
| 7 | 1 | Reverse | 32 | 1148 | AACTTATGTTGGTTATGAATAGCC |
| 7 | 1 | Reverse | 33 | 1149 | CTCGTCATGCGAAGTTGAGTTAAG |
| 7 | 1 | Reverse | 34 | 1150 | CAACCTTGCGGTTTAGCAGACTTG |
| 7 | 1 | Reverse | 35 | 1151 | GAAAGTTGTAGCTTAGGAACGACC |
| 7 | 1 | Reverse | 36 | 1152 | CTACGAAGAATCCTTAACACGTCA |
| 7 | 1 | Reverse | 37 | 1153 | TGCTTTAAATGCTAATCCAGTCGC |
| 7 | 1 | Reverse | 38 | 1154 | TGTCCTTGGGCGAGAGGCGTCCTG |
| 7 | 1 | Reverse | 39 | 1155 | TAAGTCTTTACGCAGACAACTGCC |
| 7 | 1 | Reverse | 40 | 1156 | GCGCTTATATTATCGTTCATCAAC |
| 7 | 1 | Reverse | 41 | 1157 | TACTCAATCAATCCGTGTGTAGTA |
| 7 | 1 | Reverse | 42 | 1158 | GATCCGAACATTGTCTAACCGATC |
| 7 | 1 | Reverse | 43 | 1159 | AGTTAGATACACTGTGACAGGTGG |
| 7 | 1 | Reverse | 44 | 1160 | CGAGTTGTATTCTGCAATGAGTCG |
| 7 | 1 | Reverse | 45 | 1161 | TTGCATTGCTGTACTCACAGGGAC |
| 7 | 1 | Reverse | 46 | 1162 | GGCTGTAAGGACGGATCAAGGCGC |
| 7 | 1 | Reverse | 47 | 1163 | GATCGCATGCAGCGATATCCAGAA |
| 7 | 1 | Reverse | 48 | 1164 | ACGGCGCATATTTAGTTGACTGAC |
| 7 | 1 | Reverse | 49 | 1165 | AATGTGTTCTGTGGAAGAGGATGT |
| 7 | 1 | Reverse | 50 | 1166 | GGTGGGACGGAATAGAAGCACATA |
| 7 | 1 | Reverse | 51 | 1167 | TTTAATCGACGCGCACATGTATGC |
| 7 | 1 | Reverse | 52 | 1168 | CAGGGCGCTGGAAGTTACTCAGCA |
| 7 | 1 | Reverse | 53 | 1169 | TGTACAATAAGGAGGCATTCGAAC |
| 7 | 1 | Reverse | 54 | 1170 | CAGTTCCCGGCACGCATTAAGCCC |
| 7 | 1 | Reverse | 55 | 1171 | GGATTCAATTGTAGTCGATACATG |
| 7 | 1 | Reverse | 56 | 1172 | CTCACCATTAAGTAGAACCGGAGT |
| 7 | 1 | Reverse | 57 | 1173 | GTATAGATTTCGTCTGCCTGTATT |
| 7 | 1 | Reverse | 58 | 1174 | CTAGGAGCTCGGCCAGCTTGAATG |
| 7 | 1 | Reverse | 59 | 1175 | CAGAAAGCTGCGAATAGTTTGATG |
| 7 | 1 | Reverse | 60 | 1176 | TGGGTATTCTATCGCGCGTAAGCA |
| 7 | 1 | Reverse | 61 | 1177 | GACCCAGAATTACGCCTCCAACCT |
| 7 | 1 | Reverse | 62 | 1178 | TGTACTCCGGCCACCCTAAACTTT |
| 7 | 1 | Reverse | 63 | 1179 | ATTGGCGACGGGACTATTTCTTAA |
| 7 | 1 | Reverse | 64 | 1180 | TCTAGGCCCGGAGAGATAGTATCA |

TABLE 20

Tag set 8: 24-mer non-cross-reactive oligonucleotide tags having sequences that hybridize with the reverse of the sequences generated using base oligonucleotide #2

| Run | Base | Transform | Parent SEQ ID NO | SEQ ID NO | Sequence |
|---|---|---|---|---|---|
| 8 | 2 | Reverse | 65 | 1181 | TCATGCATAGTTTCCTTACTAGTT |
| 8 | 2 | Reverse | 66 | 1182 | AGGTCTAGGAGACAGAAACGTATT |
| 8 | 2 | Reverse | 67 | 1183 | ACTCAGAAAGGCGAATCGATGACC |
| 8 | 2 | Reverse | 68 | 1184 | TCGGTAAGTTAACCATATGCCTCA |
| 8 | 2 | Reverse | 69 | 1185 | ACGTGATAAACATCCATCCATACG |
| 8 | 2 | Reverse | 70 | 1186 | CTAATTTATCGAAACGGCCAACAC |
| 8 | 2 | Reverse | 71 | 1187 | AAGCGTGTTTCATGACGAGCAGGG |
| 8 | 2 | Reverse | 72 | 1188 | TTAATGCTGATTTCGAGAGCGCCG |
| 8 | 2 | Reverse | 73 | 1189 | AAAGTCGAGAGTAACCCGTAACGT |
| 8 | 2 | Reverse | 74 | 1190 | TCTGAGATAAGGACTCCTGGACTG |
| 8 | 2 | Reverse | 75 | 1191 | TCAGGTTAGTGAAAGGAAGTGAGG |
| 8 | 2 | Reverse | 76 | 1192 | TGGCGTTGCTGAGCGAAAGAGTAA |
| 8 | 2 | Reverse | 77 | 1193 | TAGGGCCTGCCGATACAGTTCGCC |
| 8 | 2 | Reverse | 78 | 1194 | GTACACAACTAGCCTCTGGCGACT |
| 8 | 2 | Reverse | 79 | 1195 | CATATAGTAGGCTGCACAGATAGA |
| 8 | 2 | Reverse | 80 | 1196 | AGTTGACGTCGCTCGCTTAAGGAA |
| 8 | 2 | Reverse | 81 | 1197 | AACATATCCAGGGAGGTGATCCAT |
| 8 | 2 | Reverse | 82 | 1198 | ATGGGCTAAGAATAGTTTGAGATG |
| 8 | 2 | Reverse | 83 | 1199 | CAGATCTTGGTAGTAGTTGGACAG |
| 8 | 2 | Reverse | 84 | 1200 | TAGTCCCAGTTACGGTACCTTCCA |
| 8 | 2 | Reverse | 85 | 1201 | GGTACACTTGCGTATCCGACGCTT |
| 8 | 2 | Reverse | 86 | 1202 | AAGTGGACAACGGCGACATATATA |
| 8 | 2 | Reverse | 87 | 1203 | GCGGGCTCATAACGAATCCGGCTG |
| 8 | 2 | Reverse | 88 | 1204 | CCGCAACACAGCCCATAGCAGCTA |
| 8 | 2 | Reverse | 89 | 1205 | ACCTGCTTATTTGTAACGATCGGG |
| 8 | 2 | Reverse | 90 | 1206 | AACTTCGATGTCTGTACACGTACT |
| 8 | 2 | Reverse | 91 | 1207 | ATGAATGACAGACCTACCTGTCCA |
| 8 | 2 | Reverse | 92 | 1208 | CTACTGTGGTTAGACCGATCTGAG |
| 8 | 2 | Reverse | 93 | 1209 | GAACCTCGGAACTAGATCTTACTT |
| 8 | 2 | Reverse | 94 | 1210 | TGGAATCTTGAAAGGTCACTCACC |
| 8 | 2 | Reverse | 95 | 1211 | CAAGACCAAGCAGTCAGCGGATTT |
| 8 | 2 | Reverse | 96 | 1212 | GATGTAGGCTCCTCGTTCTATGTT |
| 8 | 2 | Reverse | 97 | 1213 | CATTACTCCTGTGTTAGCTAGTCA |
| 8 | 2 | Reverse | 98 | 1214 | GTTCAATCGTGACTTCCGATGTGT |
| 8 | 2 | Reverse | 99 | 1215 | TGGCTGGAACACTCAGGATTTGTA |
| 8 | 2 | Reverse | 100 | 1216 | CTCTTACCACTAGGCACACTAATA |
| 8 | 2 | Reverse | 101 | 1217 | CAATTAGCATACGCCGTACTTGTT |
| 8 | 2 | Reverse | 102 | 1218 | TTGAGTTAGAGCACTGTCGTATGT |
| 8 | 2 | Reverse | 103 | 1219 | ACCAGTTTGGAAGTGTTTGTTGAG |
| 8 | 2 | Reverse | 104 | 1220 | AGCTGCTTGAATGTGAACCAGCAT |
| 8 | 2 | Reverse | 105 | 1221 | ATGTACAGGAGAATGGTGGGAAGT |
| 8 | 2 | Reverse | 106 | 1222 | CGAAACCTACGAGTTATATGTGAT |
| 8 | 2 | Reverse | 107 | 1223 | GTCTCTTTGCTCCAAGTCCTAGAG |
| 8 | 2 | Reverse | 108 | 1224 | TAACTACACCGTGGGTTTGCGTTC |
| 8 | 2 | Reverse | 109 | 1225 | GAAAGAATCCAACGTAGTTAAATC |
| 8 | 2 | Reverse | 110 | 1226 | CTACGTGCAGTAATCAGATGATAT |
| 8 | 2 | Reverse | 111 | 1227 | CTAAGCCCTTTGTCTGACACGAAG |
| 8 | 2 | Reverse | 112 | 1228 | ATCATTGCATTGTGTCAAATTAAT |
| 8 | 2 | Reverse | 113 | 1229 | TGTTGGCGTCAATTCTATTGTGAT |
| 8 | 2 | Reverse | 114 | 1230 | GTGGCTGTACTCTATATTGTATCT |
| 8 | 2 | Reverse | 115 | 1231 | AAGCATTGTTGTTGCCGCAAAGCA |
| 8 | 2 | Reverse | 116 | 1232 | CACTGACCCAAACCTTAATACGAA |
| 8 | 2 | Reverse | 117 | 1233 | CCACGCACATCGTACTCTTAATAG |
| 8 | 2 | Reverse | 118 | 1234 | TGCATTGCTTGATAGTTACGCCAA |
| 8 | 2 | Reverse | 119 | 1235 | TAGGGTCTTGCCTCGGACCGGGTT |
| 8 | 2 | Reverse | 120 | 1236 | GTAGAACGTGATTCAGTCCTTCGT |
| 8 | 2 | Reverse | 121 | 1237 | CACTAAGGTGAAATATCCTGTGCC |
| 8 | 2 | Reverse | 122 | 1238 | CACTTGTCGTCCTCTTGGTTATGC |

TABLE 21

Tag set 9: 24-mer non-cross-reactive oligonucleotide tags having sequences that hybridize with the reverse of the sequences generated using base oligonucleotide #3

| Run | Base | Transform | Parent SEQ ID NO | SEQ ID NO | Sequence |
|---|---|---|---|---|---|
| 9 | 3 | Reverse | 123 | 1239 | TCCCAAACACTTAGATCCTCGTGT |
| 9 | 3 | Reverse | 124 | 1240 | CCCTCGTTATTTGTATTGGTAGGT |
| 9 | 3 | Reverse | 125 | 1241 | AAGGTAAACCTAGTAAAGCCTGGC |
| 9 | 3 | Reverse | 126 | 1242 | ACCGGGTATGAGCAAATGACCCAC |
| 9 | 3 | Reverse | 127 | 1243 | AGGTGGTGTCTCATGCTGCTAAAC |
| 9 | 3 | Reverse | 128 | 1244 | AATATAAGCGAGTCATGCTAACTG |
| 9 | 3 | Reverse | 129 | 1245 | CTGGCAAATCTACAAAGTCTTGTC |
| 9 | 3 | Reverse | 130 | 1246 | CTATGCAGCATTTGGATCTATCAG |
| 9 | 3 | Reverse | 131 | 1247 | ATAAACAGTGCAGGACATACTGGC |
| 9 | 3 | Reverse | 132 | 1248 | GTGGACAGGAACACCAAACGGATT |
| 9 | 3 | Reverse | 133 | 1249 | GTCTCGGCCATCTACATACCGTAT |
| 9 | 3 | Reverse | 134 | 1250 | ATCCCTGCCACACATCGGCTCGAT |
| 9 | 3 | Reverse | 135 | 1251 | ACTAAACCTCCTGCTCTGCGCGTA |
| 9 | 3 | Reverse | 136 | 1252 | CGATAAGCGTCAAAGGTGCCTTGA |
| 9 | 3 | Reverse | 137 | 1253 | ACACGTCTTATCGCCGTAGCAGAA |
| 9 | 3 | Reverse | 138 | 1254 | ATGCCCGAGCAGAATTGCCCTTAA |
| 9 | 3 | Reverse | 139 | 1255 | GACTGTCTGCAATAACAGATGTGT |
| 9 | 3 | Reverse | 140 | 1256 | CGGGTAGATGTGAATCGATATCTT |
| 9 | 3 | Reverse | 141 | 1257 | TTGGCGTGTATCATTTATCGAGTT |
| 9 | 3 | Reverse | 142 | 1258 | CACTCGTCGTCGATTATGGACATT |
| 9 | 3 | Reverse | 143 | 1259 | ATGCATATGGACCCTACTTGTCTG |
| 9 | 3 | Reverse | 144 | 1260 | AAAGATACTCAAACTTGTTGCAGC |
| 9 | 3 | Reverse | 145 | 1261 | TTGACAGACATTCGTGGGTTCCTA |
| 9 | 3 | Reverse | 146 | 1262 | CCTTTCACAGAGCCGGCATGAAAG |
| 9 | 3 | Reverse | 147 | 1263 | TGGGTGTTTATATCCCGACAGAAC |
| 9 | 3 | Reverse | 148 | 1264 | CGCTTTGGTCAGAAATGAAACCGG |
| 9 | 3 | Reverse | 149 | 1265 | CTAGGTGGTAAAGAACTTGACGTT |
| 9 | 3 | Reverse | 150 | 1266 | ATTGTGACCCGCAGTATCCTAACG |
| 9 | 3 | Reverse | 151 | 1267 | CGAAGTTGGACCTGACACGACAAT |
| 9 | 3 | Reverse | 152 | 1268 | AACGCTAATGATGTTTCTCATCGG |
| 9 | 3 | Reverse | 153 | 1269 | GCCTCAAACGGTGACGAAGGATAC |
| 9 | 3 | Reverse | 154 | 1270 | ACCTGCGTACCTCTCACCCATAGG |
| 9 | 3 | Reverse | 155 | 1271 | TCTACGTTATCGCAACCGACTTAC |
| 9 | 3 | Reverse | 156 | 1272 | ATGTAATAGGCTCACCCGCTCCAA |
| 9 | 3 | Reverse | 157 | 1273 | ACTAACACGTTGGGCATTGGAAAT |
| 9 | 3 | Reverse | 158 | 1274 | TGTATTCTTCTCGTACGTGAGAAC |
| 9 | 3 | Reverse | 159 | 1275 | ACTACTCTTGTATATCGGGTGTCC |
| 9 | 3 | Reverse | 160 | 1276 | CACGATGCGGAAGTGAATTCGATA |
| 9 | 3 | Reverse | 161 | 1277 | CTCTGGTAAACATTAGTGTAGCGG |
| 9 | 3 | Reverse | 162 | 1278 | TTATTCTAAGTCGACCTCATCTCG |
| 9 | 3 | Reverse | 163 | 1279 | ATTACCCAGTAATCTAAGCTTCCT |
| 9 | 3 | Reverse | 164 | 1280 | TACGAAACAGGTGATAATTGCAGC |
| 9 | 3 | Reverse | 165 | 1281 | CTATTGACTAAACGAAAGCCCTCA |
| 9 | 3 | Reverse | 166 | 1282 | TGAGATAGCCCAACCCATCAAGAA |
| 9 | 3 | Reverse | 167 | 1283 | TACCGACATCCTGTATCAACATTC |
| 9 | 3 | Reverse | 168 | 1284 | TCCATGCGATTGTCCTGTTTAGGT |
| 9 | 3 | Reverse | 169 | 1285 | GTATAGACAATTCAGTAAGGAGGC |
| 9 | 3 | Reverse | 170 | 1286 | GGAGCCTCATCAACCTAGGACTAC |
| 9 | 3 | Reverse | 171 | 1287 | ATTTGTAACGCCCTTCGAATTGAT |
| 9 | 3 | Reverse | 172 | 1288 | CCAATTAGGGATTATCTAGAGTGA |
| 9 | 3 | Reverse | 173 | 1289 | GTTTAACGACCGCAACCATTAGAC |
| 9 | 3 | Reverse | 174 | 1290 | GCCGACAGTTATACACTTAAGGCG |
| 9 | 3 | Reverse | 175 | 1291 | ACGTCAGCCACGAGAGAATGAGAT |
| 9 | 3 | Reverse | 176 | 1292 | AAGGGTAACTAGGCGGCTCGTAAT |
| 9 | 3 | Reverse | 177 | 1293 | AGCCATCGTGTCGTCTGGAATCCA |
| 9 | 3 | Reverse | 178 | 1294 | TAGTAGCGCGAAAGTTTGCCCAAT |
| 9 | 3 | Reverse | 179 | 1295 | AGAACAGACCCTCGATCGTTTAAG |
| 9 | 3 | Reverse | 180 | 1296 | ATTTCACAACCATACCGGGAGATT |
| 9 | 3 | Reverse | 181 | 1297 | TACCTAGGCACGGCTTAGTCTAGC |
| 9 | 3 | Reverse | 182 | 1298 | CTCATAGCCGTGAAGGTGTAGGAC |
| 9 | 3 | Reverse | 183 | 1299 | ATCGAACCTAGAGCACGTTAATCC |
| 9 | 3 | Reverse | 184 | 1300 | GGCAGCGAGTGTAAAGGACCTCTG |
| 9 | 3 | Reverse | 185 | 1301 | GCTTTAATCCGCCTACGATGCCCT |
| 9 | 3 | Reverse | 186 | 1302 | ACTCACACAATTACTACAGAGCTA |

TABLE 22

Tag set 10: 24-mer non-cross-reactive oligonucleotide tags that hybridize with the inverse complement of the sequences generated using base oligonucleotide #1

| Run | Base | Transform | Parent SEQ ID NO | SEQ ID NO | Sequence |
|---|---|---|---|---|---|
| 10 | 1 | Inverse complement | 1 | 1303 | TGACCATTGGGTCTGTACTAGCCA |
| 10 | 1 | Inverse complement | 2 | 1304 | GTAAACCAGTAACCAAGTTCTGCT |
| 10 | 1 | Inverse complement | 3 | 1305 | GATTATCGAGGACACGGGAGCATA |
| 10 | 1 | Inverse complement | 4 | 1306 | CACCGTTGTCTTTAGTCCACCACT |
| 10 | 1 | Inverse complement | 5 | 1307 | ATCTACGGCGACGTCCATACCTTT |
| 10 | 1 | Inverse complement | 6 | 1308 | TGAGGGACACCCACTCGAATTACC |
| 10 | 1 | Inverse complement | 7 | 1309 | GACCAGGCAACACCAGGAAGATTG |
| 10 | 1 | Inverse complement | 8 | 1310 | TTAGGCAGCTGATCGGACTCTTAA |
| 10 | 1 | Inverse complement | 9 | 1311 | TCTCCTGACGATTTCCAAACATCC |
| 10 | 1 | Inverse complement | 10 | 1312 | GCATGGTAACTTAGACCTCTGGAA |
| 10 | 1 | Inverse complement | 11 | 1313 | TAAGGAGTGTCTCTTGTCTGTCAA |
| 10 | 1 | Inverse complement | 12 | 1314 | GTAATTTGCTGTTACCAATGCACT |
| 10 | 1 | Inverse complement | 13 | 1315 | AGCCCTATCGTAGTAGACCGTATC |
| 10 | 1 | Inverse complement | 14 | 1316 | CTTATCCATCAACACTGCATCCCT |
| 10 | 1 | Inverse complement | 15 | 1317 | GGGCGAACCAATCGATCTATCGTG |
| 10 | 1 | Inverse complement | 16 | 1318 | GTGATACCGTCATTGGGAATCATA |
| 10 | 1 | Inverse complement | 17 | 1319 | GAACCACAGATCTTTCTTTGTGGA |
| 10 | 1 | Inverse complement | 18 | 1320 | CTTTCGTTTGCAACGAGGCAGAGC |
| 10 | 1 | Inverse complement | 19 | 1321 | TTTGTGAAATACATTCTAAGGCAG |
| 10 | 1 | Inverse complement | 20 | 1322 | ATGGGCTGAATTTAGTGCCATGAC |
| 10 | 1 | Inverse complement | 21 | 1323 | GGTTTAGGCCATATTGCATCTCTG |
| 10 | 1 | Inverse complement | 22 | 1324 | CGAAAGGGCGTCAGAGTTTCTTTC |
| 10 | 1 | Inverse complement | 23 | 1325 | GTGACTCGGTTCGGCACATTCATG |
| 10 | 1 | Inverse complement | 24 | 1326 | CAATAAATGGATGTCTTGTGGCTC |
| 10 | 1 | Inverse complement | 25 | 1327 | CCGGTCCAAGCGAACCCTACAATG |
| 10 | 1 | Inverse complement | 26 | 1328 | AAGCGCTGACAACGTCGTGATGGG |
| 10 | 1 | Inverse complement | 27 | 1329 | ATACACACACTATTTACTGACTGA |
| 10 | 1 | Inverse complement | 28 | 1330 | GCTTCCCACTACTCGTAGAAACTT |
| 10 | 1 | Inverse complement | 29 | 1331 | TCCCTGTCCGCGTGAGCTCTGATA |
| 10 | 1 | Inverse complement | 30 | 1332 | CATTATGTCCATCTAGCGCATCCT |
| 10 | 1 | Inverse complement | 31 | 1333 | ACACTGCCTAGTCGAAGGCGTCTT |
| 10 | 1 | Inverse complement | 32 | 1334 | CCAACATAAGTTCGCCGCCTTGGT |
| 10 | 1 | Inverse complement | 33 | 1335 | TCGCATGACGAGCATTTGCGAGTG |
| 10 | 1 | Inverse complement | 34 | 1336 | ACCGCAAGGTTGTTATTGTGGGAC |
| 10 | 1 | Inverse complement | 35 | 1337 | GCTACAACTTTCAAGTAAACTCGC |
| 10 | 1 | Inverse complement | 36 | 1338 | GATTCTTCGTAGACGGGTTATGGT |
| 10 | 1 | Inverse complement | 37 | 1339 | GCATTTAAAGCACGTACCGTTCTA |
| 10 | 1 | Inverse complement | 38 | 1340 | CGCCCAAGGACAAATTCATCATAT |
| 10 | 1 | Inverse complement | 39 | 1341 | CGTAAAGACTTACATCCCTAGATG |
| 10 | 1 | Inverse complement | 40 | 1342 | TAATATAAGCGCGTGAATCGTGTG |
| 10 | 1 | Inverse complement | 41 | 1343 | ATTGATTGAGTAATAGACGAACGC |
| 10 | 1 | Inverse complement | 42 | 1344 | AATGTTCGGATCCTCGCACTATGA |
| 10 | 1 | Inverse complement | 43 | 1345 | GTGTATCTAACTGCTTAACATACC |
| 10 | 1 | Inverse complement | 44 | 1346 | GAATACAACTCGTTGGGCAGTACA |
| 10 | 1 | Inverse complement | 45 | 1347 | ACAGCAATGCAAATAATTGGCGGC |
| 10 | 1 | Inverse complement | 46 | 1348 | GTCCTTACAGCCTTTATGAACTTT |
| 10 | 1 | Inverse complement | 47 | 1349 | CTGCATGCGATCAGATCAATGCTT |
| 10 | 1 | Inverse complement | 48 | 1350 | AATATGCGCCGTGGCTTAAGGTGA |
| 10 | 1 | Inverse complement | 49 | 1351 | ACAGAACACATTCCTACTCCGTTT |
| 10 | 1 | Inverse complement | 50 | 1352 | TTCCGTCCCACCCAACAACTATAG |
| 10 | 1 | Inverse complement | 51 | 1353 | GCGTCGATTAAATTGATATAGACC |
| 10 | 1 | Inverse complement | 52 | 1354 | TCCAGCGCCCTGCCCATTATAAGT |
| 10 | 1 | Inverse complement | 53 | 1355 | CCTTATTGTACAGGGTTAACTACT |
| 10 | 1 | Inverse complement | 54 | 1356 | TGCCGGGAACTGAAACAAGATATT |
| 10 | 1 | Inverse complement | 55 | 1357 | ACAATTGAATCCCGGATGACTCCG |
| 10 | 1 | Inverse complement | 56 | 1358 | CTTAATGGTGAGGTAACCGGACGA |
| 10 | 1 | Inverse complement | 57 | 1359 | CGAAATCTATACTCGGCTGTGAAT |
| 10 | 1 | Inverse complement | 58 | 1360 | CCGAGCTCCTAGTAGTATAGTTAC |
| 10 | 1 | Inverse complement | 59 | 1361 | CGCAGCTTTCTGCGGTCATGAAGG |
| 10 | 1 | Inverse complement | 60 | 1362 | ATAGAATACCCAAAGAGCATATAA |
| 10 | 1 | Inverse complement | 61 | 1363 | TAATTCTGGGTCCATCATCGACAA |
| 10 | 1 | Inverse complement | 62 | 1364 | GGCCGGAGTACAATCTAGTCACCC |
| 10 | 1 | Inverse complement | 63 | 1365 | CCCGTCGCCAATCATGCTCCAGCC |
| 10 | 1 | Inverse complement | 64 | 1366 | TCCGGGCCTAGAAACAGTGCTTGC |

TABLE 23

Tag set 11: 24-mer non-cross-reactive oligonucleotide tags that hybridize with the inverse complement of the sequences generated using base oligonucleotide #2

| Run | Base | Transform | Parent SEQ ID NO | SEQ ID NO | Sequence |
|---|---|---|---|---|---|
| 11 | 2 | Inverse complement | 65 | 1367 | AACTATGCATGAGACTTGCTGCTG |
| 11 | 2 | Inverse complement | 66 | 1368 | TCTCCTAGACCTCCGGCACGACAA |
| 11 | 2 | Inverse complement | 67 | 1369 | GCCTTTCTGAGTCGCAAAGTCATT |
| 11 | 2 | Inverse complement | 68 | 1370 | TTAACTTACCGAGCAAGCTTCTTA |
| 11 | 2 | Inverse complement | 69 | 1371 | TGTTTATCACGTGCGCGAGAGATT |
| 11 | 2 | Inverse complement | 70 | 1372 | TCGATAAATTAGAGTTCTGGTCTA |
| 11 | 2 | Inverse complement | 71 | 1373 | TGAAACACGCTTGTCTAATTCGAT |
| 11 | 2 | Inverse complement | 72 | 1374 | AATCAGCATTAACTCTTTAACGCC |
| 11 | 2 | Inverse complement | 73 | 1375 | ACTCTCGACTTTATATCACGCCAG |
| 11 | 2 | Inverse complement | 74 | 1376 | CCTTATCTCAGATTTGTTCGGCGA |
| 11 | 2 | Inverse complement | 75 | 1377 | TCACTAACCTGACAGCAACCATAC |
| 11 | 2 | Inverse complement | 76 | 1378 | TCAGCAACGCCACTAAAGCTATAT |
| 11 | 2 | Inverse complement | 77 | 1379 | CGGCAGGCCCTAAAGAAATTTGTA |
| 11 | 2 | Inverse complement | 78 | 1380 | CTAGTTGTGTACTATCATATCGAC |
| 11 | 2 | Inverse complement | 79 | 1381 | GCCTACTATATGGTGCGACAATAA |
| 11 | 2 | Inverse complement | 80 | 1382 | GCGACGTCAACTGGAAGATGTATG |
| 11 | 2 | Inverse complement | 81 | 1383 | CCTGGATATGTTCAGTTTATCGCT |
| 11 | 2 | Inverse complement | 82 | 1384 | TTCTTAGCCCATGGTTGGGTGAAA |
| 11 | 2 | Inverse complement | 83 | 1385 | TACCAAGATCTGTTGATTACGCAG |
| 11 | 2 | Inverse complement | 84 | 1386 | TAACTGGGACTACCACAATTATCA |
| 11 | 2 | Inverse complement | 85 | 1387 | CGCAAGTGTACCTGGATTGAGCAT |
| 11 | 2 | Inverse complement | 86 | 1388 | CGTTGTCCACTTTCTGGAACTGAT |
| 11 | 2 | Inverse complement | 87 | 1389 | TTATGAGCCCGCACGATTATTATA |
| 11 | 2 | Inverse complement | 88 | 1390 | GCTGTGTTGCGGTGCATCTAAGTA |
| 11 | 2 | Inverse complement | 89 | 1391 | AAATAAGCAGGTGCCACTCATTGA |
| 11 | 2 | Inverse complement | 90 | 1392 | GACATCGAAGTTCAAAGCGCTCCC |
| 11 | 2 | Inverse complement | 91 | 1393 | TCTGTCATTCATCTCCTTAGTCAG |
| 11 | 2 | Inverse complement | 92 | 1394 | TAACCACAGTAGGACGGACTAACA |
| 11 | 2 | Inverse complement | 93 | 1395 | GTTCCGAGGTTCCGAATGCGAGGT |
| 11 | 2 | Inverse complement | 94 | 1396 | TTCAAGATTCCAATTCTGCGAAGC |
| 11 | 2 | Inverse complement | 95 | 1397 | TGCTTGGTCTTGGGACTTACAAGC |
| 11 | 2 | Inverse complement | 96 | 1398 | GGAGCCTACATCTCGCTAACTCCT |
| 11 | 2 | Inverse complement | 97 | 1399 | ACAGGAGTAATGGCAGGGTTGTGT |
| 11 | 2 | Inverse complement | 98 | 1400 | TCACGATTGAACAAACTCATTCGA |
| 11 | 2 | Inverse complement | 99 | 1401 | GTGTTCCAGCCACGACGTTAAGCT |
| 11 | 2 | Inverse complement | 100 | 1402 | TAGTGGTAAGAGAGAGTGAAGCGA |
| 11 | 2 | Inverse complement | 101 | 1403 | GTATGCTAATTGGGAAGGCCAGGT |
| 11 | 2 | Inverse complement | 102 | 1404 | GCTCTAACTCAATCTCAGGGATAA |
| 11 | 2 | Inverse complement | 103 | 1405 | TTCCAAACTGGTAAACTGCTCGGA |
| 11 | 2 | Inverse complement | 104 | 1406 | ATTCAAGCAGCTCATCTTGCCAGG |
| 11 | 2 | Inverse complement | 105 | 1407 | TCTCCTGTACATCGTTGACAGCGT |
| 11 | 2 | Inverse complement | 106 | 1408 | TCGTAGGTTTCGTGCTAAGTGCCG |
| 11 | 2 | Inverse complement | 107 | 1409 | GAGCAAAGAGACTCATCGTCTATC |
| 11 | 2 | Inverse complement | 108 | 1410 | ACGGTGTAGTTATTAGGGTCCTAG |
| 11 | 2 | Inverse complement | 109 | 1411 | TTGGATTCTTTCGACACCAAGGCG |
| 11 | 2 | Inverse complement | 110 | 1412 | TACTGCACGTAGATGGCGTGTCTA |
| 11 | 2 | Inverse complement | 111 | 1413 | CAAAGGGCTTAGCTTATTGCAGCC |
| 11 | 2 | Inverse complement | 112 | 1414 | CAATGCAATGATGGGTCGCGGTCT |
| 11 | 2 | Inverse complement | 113 | 1415 | TTGACGCCAACAAGACGCGGGTGA |
| 11 | 2 | Inverse complement | 114 | 1416 | GAGTACAGCCACAGGGATGCATGC |
| 11 | 2 | Inverse complement | 115 | 1417 | ACAACAATGCTTCTGCCGCTTTAC |
| 11 | 2 | Inverse complement | 116 | 1418 | TTTGGGTCAGTGAGGCAGTGAGCA |
| 11 | 2 | Inverse complement | 117 | 1419 | CGATGTGCGTGGCGCGACTTCATG |
| 11 | 2 | Inverse complement | 118 | 1420 | TCAAGCAATGCACAAGTTCTCGGT |
| 11 | 2 | Inverse complement | 119 | 1421 | GGCAAGACCCTAGAATAACAAATT |
| 11 | 2 | Inverse complement | 120 | 1422 | ATCACGTTCTACCTCTCACAAGTA |
| 11 | 2 | Inverse complement | 121 | 1423 | TTCACCTTAGTGTCTTAGACTCCC |
| 11 | 2 | Inverse complement | 122 | 1424 | GGACGACAAGT GCATATAC TACAG |

TABLE 24

Tag set 12: 24-mer non-cross-reactive oligonucleotide tags that hybridize with the inverse complement of the sequences generated using base oligonucleotide #3

| Run | Base | Transform | Parent SEQ ID NO | SEQ ID NO | Sequence |
|---|---|---|---|---|---|
| 12 | 3 | Inverse complement | 123 | 1425 | AAGTGTTTGGGAGAGGTGTCATCG |
| 12 | 3 | Inverse complement | 124 | 1426 | AAATAACGAGGGATCGTAACGACA |
| 12 | 3 | Inverse complement | 125 | 1427 | TAGGTTTACCTTTCTCTTGCAGAG |
| 12 | 3 | Inverse complement | 126 | 1428 | CTCATACCCGGTGTTGAAGACCAA |
| 12 | 3 | Inverse complement | 127 | 1429 | GAGACACCACCTCAATGCTAAGCG |
| 12 | 3 | Inverse complement | 128 | 1430 | CTCGCTTATATTCCCGATGCTGGC |
| 12 | 3 | Inverse complement | 129 | 1431 | TAGATTTGCCAGCGTTCGCCTTGG |
| 12 | 3 | Inverse complement | 130 | 1432 | AATGCTGCATAGTGCGCTTGTCGG |
| 12 | 3 | Inverse complement | 131 | 1433 | TGCACTGTTTATGATCTGTACCAA |
| 12 | 3 | Inverse complement | 132 | 1434 | GTTCCTGTCCACACCGCTTCATGT |
| 12 | 3 | Inverse complement | 133 | 1435 | GATGGCCGAGACTTCCTGGGATCA |
| 12 | 3 | Inverse complement | 134 | 1436 | TGTGGCAGGGATATGGTCACATTT |
| 12 | 3 | Inverse complement | 135 | 1437 | AGGAGGTTTAGTATAAATTGGGTG |
| 12 | 3 | Inverse complement | 136 | 1438 | TGACGCTTATCGGCAGTCTTGAAA |
| 12 | 3 | Inverse complement | 137 | 1439 | GATAAGACGTGTGCTTCCGTTAGA |
| 12 | 3 | Inverse complement | 138 | 1440 | CTGCTCGGGCATAGCGGAACCTTA |
| 12 | 3 | Inverse complement | 139 | 1441 | TTGCAGACAGTCCGCTCCGAATAT |
| 12 | 3 | Inverse complement | 140 | 1442 | CACATCTACCCGCAATTCGGTCGT |
| 12 | 3 | Inverse complement | 141 | 1443 | GATACACGCCAAATATTGGAGGGC |
| 12 | 3 | Inverse complement | 142 | 1444 | CGACGACGAGTGTCCGCTTGACTA |
| 12 | 3 | Inverse complement | 143 | 1445 | GTCCATATGCATCAACTATGGAAA |
| 12 | 3 | Inverse complement | 144 | 1446 | TTGAGTATCTTTGATAGGGACCTC |
| 12 | 3 | Inverse complement | 145 | 1447 | AATGTCTGTCAAGCAGTTTATCTC |
| 12 | 3 | Inverse complement | 146 | 1448 | CTCTGTGAAAGGAAGTATATTGAG |
| 12 | 3 | Inverse complement | 147 | 1449 | TATAAACACCCATGTGCTGGTCTG |
| 12 | 3 | Inverse complement | 148 | 1450 | CTGACCAAAGCGATAGACCTACAA |
| 12 | 3 | Inverse complement | 149 | 1451 | TTTACCACCTAGCTCGCGAAATCA |
| 12 | 3 | Inverse complement | 150 | 1452 | GCGGGTCACAATCTGATGTACAGG |
| 12 | 3 | Inverse complement | 151 | 1453 | GGTCCAACTTCGCTCAAGCTTCTA |
| 12 | 3 | Inverse complement | 152 | 1454 | ATCATTAGCGTTAACTCGTGCCCT |
| 12 | 3 | Inverse complement | 153 | 1455 | ACCGTTTGAGGCCGCTCTTTAACT |
| 12 | 3 | Inverse complement | 154 | 1456 | AGGTACGCAGGTATCATATCACTA |
| 12 | 3 | Inverse complement | 155 | 1457 | CGATAACGTAGATTAGTTTGGCAC |
| 12 | 3 | Inverse complement | 156 | 1458 | AGCCTATTACATAAGTCTCTCATC |
| 12 | 3 | Inverse complement | 157 | 1459 | CAACGTGTTAGTTGAATGGTCGCC |
| 12 | 3 | Inverse complement | 158 | 1460 | GAGAAGAATACAGCGAAATAAGGA |
| 12 | 3 | Inverse complement | 159 | 1461 | TACAAGAGTAGTGAATCCTCAGTC |
| 12 | 3 | Inverse complement | 160 | 1462 | TTCCGCATCGTGATTGCTTGCTAA |
| 12 | 3 | Inverse complement | 161 | 1463 | TGTTTACCAGAGGCTATGTCCAAC |
| 12 | 3 | Inverse complement | 162 | 1464 | GACTTAGAATAACGTTTGCAACTA |
| 12 | 3 | Inverse complement | 163 | 1465 | TTACTGGGTAATGGCCTCGTATCA |
| 12 | 3 | Inverse complement | 164 | 1466 | ACCTGTTTCGTAAGCAGCTACATC |
| 12 | 3 | Inverse complement | 165 | 1467 | TTTAGTCAATAGTGTGGTGCTATT |
| 12 | 3 | Inverse complement | 166 | 1468 | TGGGCTATCTCAGTGACATCTAGG |
| 12 | 3 | Inverse complement | 167 | 1469 | AGGATGTCGGTAGGCCAGTCCTTA |
| 12 | 3 | Inverse complement | 168 | 1470 | CAATCGCATGGAGTCGCAGTCGTA |
| 12 | 3 | Inverse complement | 169 | 1471 | AATTGTCTATACCGGCGATTAACA |
| 12 | 3 | Inverse complement | 170 | 1472 | TGATGAGGCTCCTAAGAAATACGT |
| 12 | 3 | Inverse complement | 171 | 1473 | GGCGTTACAAATTGACCGGACAGG |
| 12 | 3 | Inverse complement | 172 | 1474 | ATCCCTAATTGGTGGCCCGCGTTT |
| 12 | 3 | Inverse complement | 173 | 1475 | CGGTCGTTAAACGTCTGTGGTTGG |
| 12 | 3 | Inverse complement | 174 | 1476 | ATAACTGTCGGCCTCTATGTCGCA |
| 12 | 3 | Inverse complement | 175 | 1477 | CGTGGCTGACGTCTGATTTCACCT |
| 12 | 3 | Inverse complement | 176 | 1478 | CTAGTTACCCTTCGGAATTAATGA |
| 12 | 3 | Inverse complement | 177 | 1479 | GACACGATGGCTTCCATATAAGAC |
| 12 | 3 | Inverse complement | 178 | 1480 | TTCGCGCTACTAGTTGTCAAAGCC |
| 12 | 3 | Inverse complement | 179 | 1481 | AGGGTCTGTTCTAGCGGCTCAGTA |
| 12 | 3 | Inverse complement | 180 | 1482 | TGGTTGTGAAATTGCATCACTGGG |
| 12 | 3 | Inverse complement | 181 | 1483 | CGTGCCTAGGTATTGCCACAACTT |
| 12 | 3 | Inverse complement | 182 | 1484 | CACGGCTATGAGGTATGTCTGACT |
| 12 | 3 | Inverse complement | 183 | 1485 | TCTAGGTTCGATGATAACGGGCGT |
| 12 | 3 | Inverse complement | 184 | 1486 | ACACTCGCTGCCATTTGGCCTTAA |
| 12 | 3 | Inverse complement | 185 | 1487 | GCGGATTAAAGCGTGTTGTGTTGA |
| 12 | 3 | Inverse complement | 186 | 1488 | AATTGTGTGAGTCGGTCCGTTGCA |

TABLE 25

Thiol modified capture oligonucleotides (36-mer)

| Thiol modified oligonucleotides | SEQ ID NO: |
|---|---|
| 5′-ACC GAT CAT GTC TGG GTT ACC AGT TAG TCG TGT CTC /iSp18//3ThioMC3-D/-3′ | 1489 |
| 5′-TCG TCT TGA ACC AAT GAC CAA ATG CAA GCC CTC CAT /iSp18//3ThioMC3-D/-3′ | 1490 |
| 5′-ATA CGA GGG CAC AGG AGC TAT TAG TGT AGC GAA AGG /iSp18//3ThioMC3-D/-3′ | 1491 |
| 5′-TCA CCA CCT GAT TTC TGT TGC CAC CGC ATC AGT TTA /iSp18//3ThioMC3-D/-3′ | 1492 |
| 5′-TTT CCA TAG CTG CAG CGG CAT CTA TTC ATG ACA TGT /iSp18//3ThioMC3-D/-3′ | 1493 |
| 5′-CCA TTA AGC TCA CCC ACA GGG AGT TGG AGT CTA AAC /iSp18//3ThioMC3-D/-3′ | 1494 |
| 5′-GTT AGA AGG ACC ACA ACG GAC CAG AGA GTG CAT ATA /iSp18//3ThioMC3-D/-3′ | 1495 |
| 5′-AAT TCT CAG GCT AGT CGA CGG ATT TAG CGT CAC TCG /iSp18//3ThioMC3-D/-3′ | 1496 |
| 5′-CCT ACA AAC CTT TAG CAG TCC TCT GTT GGT CTC TGC /iSp18//3ThioMC3-D/-3′ | 1497 |
| 5′-AAG GTC TCC AGA TTC AAT GGT ACG ACC ATC CGA CTC /iSp18//3ThioMC3-D/-3′ | 1498 |

### WORKING EXAMPLES

The present invention is not to be limited in scope by the specific embodiments described herein. Indeed, various modifications of the method in addition to those described herein will become apparent to those skilled in the art from the foregoing description and accompanying figures. Such modifications are intended to fall within the scope of the claims.

### Example 1. Selection of Non-Interacting Capture Oligonucleotides

Software was used to randomly generate groups of 100,000 to 1,000,000 nucleotide sequences. Multiple groups were created of 36-mers. Within each group, sequences were eliminated that did not meet criteria for GC content (40% ≤ GC content ≤ 50%), AG content (30% ≤ AG content ≤ 70%) and CT content (30% ≤ CT content ≤ 70%), where GC (or AG or CT) content refers to the percentage of nucleotides that are G or C (or A or G, or C or T, respectively). Sequences were also eliminated if they had stretches of base repeats that were longer than 3 bases. Within a group, a set of non-interacting sequences was selected in an iterative process starting with a first randomly selected sequence from the group. Additional sequences were added one at a time to the set based their lack of predicted interactions with sequences already in the set. Sequences were added to the set if they met the following criteria: an alignment of the sequence with itself, with a previous member of the set, or with the complement of a previous member of the set could not be found (a) where there was a consecutive series of more than 7 complementary base pair matches in a row or (b) where there was a sequence of 18 bases or less where (i) the terminal bases at each end were complementary matches and (ii) the sum of the complementary base pair matches minus the sum of the mismatches was greater than 7. Using this approach, sets of roughly 50 to 150 sequences could be identified (for example, SEQ ID NOs 1 to 64, 65 to 122 and 123 to 186). Additional sets can be created by reversing or finding the complement of all the sequences in one of the original sets (for example, SEQ ID NOs 187 to 250, 251 to 308, 309 to 372, 373 to 436, 437 to 494, 495 to 558, 559 to 622, 623 to 680, and 681 to 744). The sequences are long enough that the probability of finding a matching sequence in nature is very low. A BLAST search of selected sets against the human genome did not find any matching or complementary sequences longer than 20 base pairs. Subsets of 10 sequences and 30 sequences from one of the sets (SEQ ID NOs 1 to 10, 11 to 13, 25 to 26, 33 to 37, 42, 44 to 46, 54 and 59 to 62, respectively) were selected as having free energies of hybridization (for hybridization to 24-mer probes complementary to the first 24 nucleotides of the 36-mer sequences starting at the 35-mer 5′ end) that were roughly in the center of the distribution of free energies for the full set of 36-mers (calculated free energies ranged from roughly -24 to roughly -22 kcal/mol). The 10 oligonucleotide set was used to demonstrate use of these sequences as capture reagents in the examples below.

### Example 2. Formation of Capture Oligonucleotide Arrays

Arrays were formed on 10-Spot 96-well MULTI-ARRAY® plates (Meso Scale Diagnostics, LLC.). These 96-well plates are formed by adhering an injection molded 96-well plate top to a mylar sheet that defines the bottom of the wells. The top surface of the mylar sheet has screen printed carbon ink electrodes printed on it such that each well includes a carbon ink working electrode roughly in the center of the well and two carbon ink counter electrodes roughly towards two edges of the well. The working electrode has a dielectric (i.e., electrically insulating ink) printed over it in a pattern that defines 10 roughly circular areas of exposed working electrode (or “spots”) which define the locations of array elements. Electrodes printed on the bottom of the mylar sheet, connected through conductive through-holes to the top of the sheet provide contacts for applying electrical voltage to the working and counter electrodes. See, for example, U.S. Pat. Nos. 6,977,722 and 7,842,246 for descriptions of plates with integrated carbon-based electrodes.

Capture oligonucleotide arrays of SEQ IDS 1 to 10 were printed on these plates by depositing 50 nL droplets containing thiol-modified capture oligonucleotides (using the n-mercaptopropanol modification linked to the 3′ end of the oligonucleotide through a 6-mer polyethyleneglycol (PEG6) spacer as shown in the structure below) on individual spots on the electrodes. The printing solutions included thiol oligonucleotide in a buffered solution containing sodium phosphate, NaCl, EDTA, Trehalose, and Triton X-100, with an excess of oligonucleotide relative to amount needed to saturate the carbon ink surface, and sufficient Triton X-100 so that the droplets spread to the edge of the spot as defined by the printed dielectric ink layer. The droplets were allowed to dry overnight, during which time the oligonucleotides bound to the carbon ink surface. The plates were packaged in sealed pouches with dessicant.

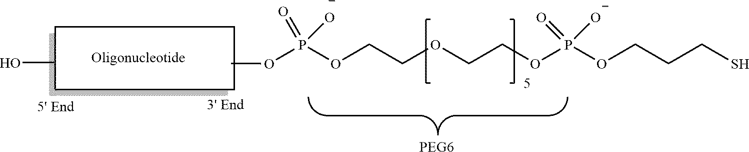

### Example 3. A Procedure for Measuring Biotin-Labeled Oligonucleotides With Sequences Complementary to Capture Oligonucleotides in an Array

In this procedure, plates with capture oligonucleotide arrays were prepared as described in Example 2, and used to measure, for example, biotin-labeled products of sandwich hybridization assays, oligonucleotide ligation assays (OLAs) and polymerase extension assays (PEAs). The procedure included an initial blocking step where the arrays were first treated with a blocking solution to dissolve excess non-immobilized capture oligonucleotide while preventing cross-contamination of non-specific spots. The overall procedure included the following steps:
1. Blocking 50 uL of a solution containing 50 mM L-cysteine and 0.1% (w/v) Triton X-100 in 20 mM Tris-HCl buffer, pH 8.0 (where Tris refers to tris(hydroxymethyl)aminomethane) was added to each well. The plates were incubated with shaking for 30 to 60 minutes at room temperature (or 37° C.). The blocking step was completed by washing the wells three times with phosphate buffered saline (PBS).
2. Addition of Sample 50 uL of a test sample containing the biotin-labeled products in a buffer containing 31% formamide, 400 mM NaCl, 1 mM EDTA, 0.01% Triton X-100 in 20 mM Tris-HCl, pH 8.0 was added to each well. After addition of the sample, the plates were incubated with shaking for one hour at 37° C. to provide stringent binding conditions, cooled at room temperature for 5 min. and washed three times with PBS.
3. Hot Soak Under Stringent Conditions (Optional) 50 uL of 0.1X PBS (concentration of salt ~ 15 mM) was added to each well and the plates were incubated with shaking for 30 min. at 37°, after which the plates were cooled at room temperature for 5 min. and washed three times with PBS. This optional step provides improved specificity in assays such as OLA assays, for example, by minimizing the non-specific binding of biotin-labeled OLA products to the wrong capture oligonucleotide, or by preventing the linking of directing sequence to biotin through non-covalent hybridization interactions.
4. Addition of Secondary Binding Reagent To detect the biotin-labeled probes, 50 uL of a solution containing 1 ug/mL of streptavidin labeled with SULFO-TAG ECL label (Meso Scale Diagnostics, LLC.) in 500 mM NaCl, 1 mM EDTA, 0.01% Triton X-100, 20 mM Tris-HCl, pH 8.0 was added to each well and the plates were incubated for 30 min. with shaking after which they were washed three time with PBS.
5. ECL Detection To measure ECL from the ECL label, 150 uL of an ECL read buffer containing butyldiethanolamine (BDEA) as the ECL coreactant (see copending Patent Application 62/787,892 , entitled COMPOSITIONS AND METHODS FOR CARRYING OUT ASSAY MEASUREMENTS, filed on Jan. 3, 2019) was added to each well and the plate was analyzed on a SECTOR Imager 600 or QuickPlex SQ120 ECL plate reader. The plate readers contacted the electrical contacts on the bottom of the plates, applied a voltage waveform across the working and counter electrodes within each well, imaged the ECL, and reported an ECL signal proportional to the total ECL emission from each array element.

### Example 4. Uniformity and Cross-Reactivity of Capture Oligonucleotide Arrays

A lot of plates prepared as described in Example 2 were tested for uniformity of coating and for cross-reactivity between array elements using a set of biotin-containing QC probes that were complementary to the first 24 nucleotides (from the 5′ end) of the capture oligonucleotides (SEQ ID NOs 745 to 754). Plates were tested according to the procedure described in Example 3, without the optional Hot Soak step. QC probes that were used included probes that were modified at the 3′ end with a biotin modification as shown in the structure below:

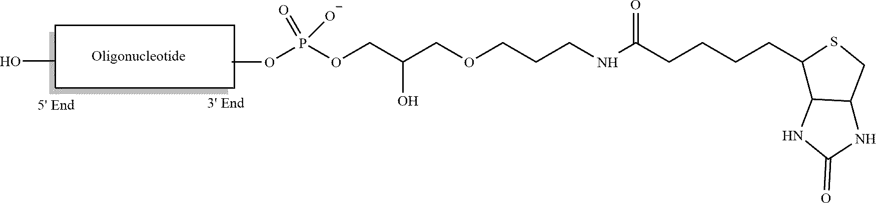

To measure uniformity of coating, all the wells of six plates were tested with a sample containing a mixture of the 10 biotin-labeled QC probes at 2 pM, as well as the non-biotin modified versions of the same probes at 2 nM. The average and the intraplate coefficient of variation (CV) was determined for the ECL signal from each capture oligonucleotide (i.e., the average and the CV for the signal from a given spot in a given plate). The average intraplate CV across the six plates were less than 5% for all of the capture oligonucleotides and ranged from 3.6% to 4.6%. The CV of the intraplate signal averages were less than 6% for all capture oligonucleotides and ranged from 3.5% to 5.5%.

To measure array specificity (including cross-reactivity from either binding of non-complementary sequences or from capture oligonucleotide cross-contamination), samples containing individual biotin-labeled QC probes at 200 pM were added to one plate (8 replicates per QC probe and 16 blank samples). The median cross-reactivity of each individual QC probe for each non-specific capture nucleotide was determined for the eight replicates of each specificity sample, where cross-reactivity was calculated for each well as the signal from the binding of a probe to a spot with a non-specific capture nucleotide as a percentage of the signal from the binding of the probe to the spot with its specific complementary capture nucleotide (after correction for non-specific background signal in the absence of any QC probe). For the 90 possible non-specific probe/capture interactions, 81 (90%) had a cross-reactivity of 0.01% or less and maximum cross-reactivity was 0.03%.

### Example 5. Comparison of Linkers for Capture Oligonucleotides

A model 12-mer capture oligonucleotide and a model 24-mer capture oligonucleotide were used to compare the use of linkers of different length between the oligonucleotide and the thiol used to link the oligonucleotide to carbon-based electrodes. The linkers included the linker with a PEG6 spacer as described in Example 2, an analogous linker except with a 3-mer polyethyleneglycol (PEG3) spacer, and a linker with no polyethyleneglycol spacer as shown below.

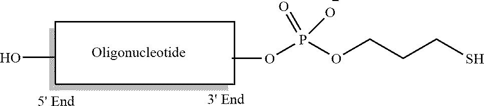

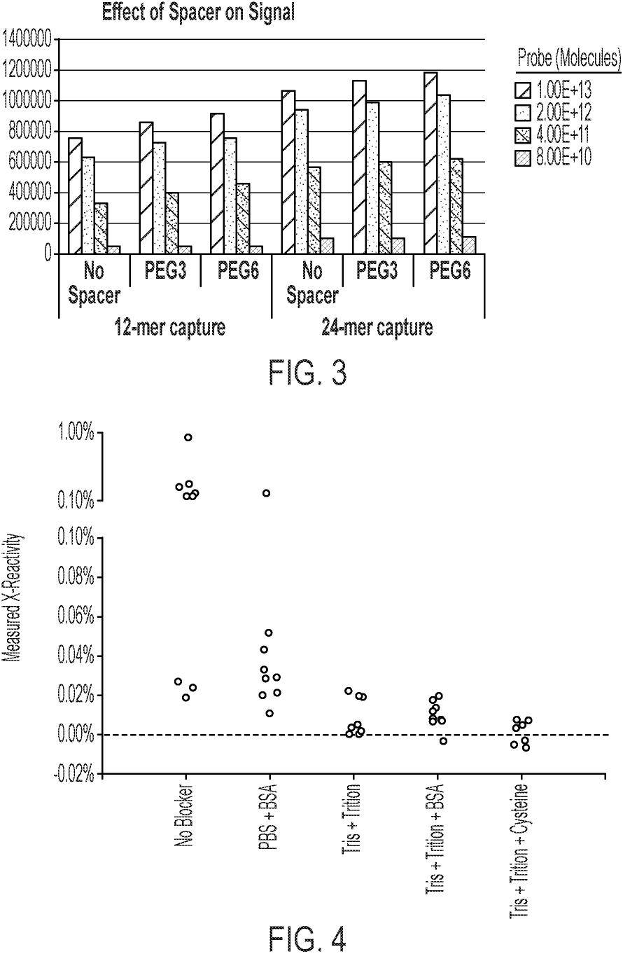

The capture oligonucleotides were immobilized on carbon electrodes in 96-well plates as described in Example 2 and tested with varying concentrations of biotin-modified QC probes complementary to the capture sequences under conditions similar to those described in Example 4 except that the hybridizations were carried out at room temperature in the absence of formamide. FIG. 3 shows the measured ECL signal as a function of the number of probe molecules in a well for the different linkers and demonstrates that the ECL signal from the binding of the QC probes to the capture oligonucleotides increased with linker length for both the 12-mer and 24-mer capture oligonucleotides.

### Example 6. Comparison of Blocking Conditions for Preparing Arrays

Blocking conditions for removing excess capture oligonucleotides from arrays were compared. Plates with printed arrays were prepared as described in Example 2 and blocked and washed as described in Example 4, except for varying the composition of the blocking solution. Specificity of the arrays was then characterized as described in Example 4. FIG. 4 shows the cross reactivity to the spot with the capture oligonucleotide with SEQ ID NO: 5 resulting from exposure to the QC probes complementary to the other capture oligonucleotides. The figure shows that omitting the blocking step leads to significant observed cross-reactivity due to cross-contamination of the capture oligonucleotides on the different spots. A conventional blocking solution with BSA in PBS provides only marginal improvement. The observed cross-reactivity is significantly improved when using Tris + Triton X-100 as the blocking solution. The addition of cysteine to the Tris/Triton formulation further reduces the cross-reactivity to non-detectable levels (≤ 0.01%). Adding BSA to the formulation, however, did not lead to the same improvement. In separate experiments, it was determined that cysteine concentrations ranging from 5 to 50 to 500 mM were effective at blocking, and it was also determined that blocking with BSA, but not cysteine, could cause a reduction in the signal from the desired interaction of the QC probes for their complementary capture oligonucleotides (data not shown).

Other blocking agents that were useful for reducing cross-contamination (data not shown), although not as effectively as thiol blocking agents like cysteine, included (i) polymers used to reduce background signals in hybridization assays including PS20, polyvinyl alcohol (PVA), polyvinylpyrrolidone (~ 1,000 kD, and ~ 360 kD), Ficoll, and polyethylene glycol (~ 3 kD and ~ 10 kD), (ii) nucleic acids and other polyanions including salmon sperm DNA, herring DNA, calf thymus DNA, sheared PolyA, yeast tRNA; and heparin, (iii) monomeric and polymeric protein blocking agents including BSA, and poly-BSA, (iv) surfactants including sodium dodecyl sulfate (SDS), 3-[(3-cholamidopropyl)dimethylammonio]-1-propanesulfonate (CHAPS), triton-100, and tween-20, and (v) hydrogen bond destabilizers such as formamide and propylene glycol.

It should be noted that the blocking and washing step can be carried out during manufacturing of arrays and prior to packaging of the arrays. The best performance, however, is achieved if this step is carried out just prior to use of the arrays. After blocking there may still be some loosely bound cross-contaminating oligonucleotides that are bound via weak base-base interactions with immobilized oligonucleotides. These would normally dissociate during the stringent hybridization conditions used in assays, but may become irreversibly immobilized if dried and stored on the array for long periods of time.

### Example 7. A Procedure for Oligonucleotide Ligation Assay (OLA) to Detect Single Nucleotide Polymorphisms (SNPs).

Detection of a SNP is carried out using a pair of oligonucleotide probes as shown in FIG. 1A: (i) a directing probe that includes a sequence towards the 5′ end that includes a sequence selected from SEQ ID NOs: 745 to 754 (i.e., a sequence that hybridizes to one of the capture oligonucleotides in the arrays prepared as described in Example 2) and a first probe sequence at the 3′ end that is complementary to the analyte nucleic acid sequence at and downstream from the SNP site (such that the 3′ end is complementary to the SNP nucleotide in the analyte) and (ii) a detection probe with a second probe sequence complementary to the analyte nucleotide sequence immediately upstream of the SNP and includes a biotin moiety at the 3′ terminus. In the presence of the analyte and a ligase, the probe pairs are ligated only when the directing probe matches the SNP nucleotide. When comparing the levels of different nucleotides at a SNP position (e.g., the levels of a wild type nucleotide vs. a mutant nucleotide), a directing probe is provided for each alternative. For some assays, the sequences of OLA directing and detection probes for SNPs include a sense DNA strand sequences of the coding region ( as for BRAF1799, NRAS181, and NRAS182), while for other assays the sequences of OLA directing and detection probes include antisense DNA strand sequences of the coding region (as for TP53, PIK3CA, KRAS, and APC).

Blocking oligonucleotide probes were developed for each of the OLA probes. The blocking probes use the matched sequence of the analyte binding portion (i.e., the first or second probe sequence). In some cases they may also have a few (e.g., 3) additional nucleotides at the 3′ or 5′ end that are complementary to the corresponding nucleotides adjacent to the target sequences on the analyte, although these additional nucleotides are generally not required to provide blocking activity.

Synthetic DNA templates were also created for each wild type and mutant target that could be used to test the performance of the probes.

The sequences of OLA probes for seven SNPs that were tested on oligo array are listed in Table 26 below with regions complementary to the capture oligonucleotides shown in bold.

TABLE 26

OLA probe sequences

| SEQ ID NO: | SNP | Reagent | Sequence |
|---|---|---|---|
| 1653 | BRAF 1799T (WT) | Dir probe-s1 | ACTGGTAACCCAGACATGATCGGTAGGTGATTTTGGTCTAGCTACAGT |
| 1654 | BRAF 1799A (M) | Dir probe-s6 | ACTCCCTGTGGGTGAGCTTAATGGAGGTGATTTTGGTCTAGCTACAGA |
| 1655 | BRAF 1799 | Det Probe | GAAATCTCGATGGAGTGGGTC |
| 1656 | BRAF 1799T (WT) | Template | TTCAAACTGATGGGACCCACTCCATCGAGATTTCACTGTAGCTAGACCAAAATCACCTATTTTTACTGTGAGGTC |
| 1657 | BRAF 1799A (M) | Template | TTCAAACTGATGGGACCCACTCCATCGAGATTTCTCTGTAGCTAGACCAAAATCACCTATTTTTACTGTGAGGTC |
| 1658 | BRAF 1799 | Block. Probe 1 | AGGTGATTTTGGTCTAGCTACAGT/A |
| 1659 | BRAF 1799 | Block. Probe 2 | GAAATCTCGATGGAGTGGGTC |
| 1660 | NRAS 181C (WT) | Dir probe-s2 | CATTTGGTCATTGGTTCAAGACGAGACATACTGGATACAGCTGGAC |
| 1661 | NRAS 181A (M) | Dir probe-s7 | CTGGTCCGTTGTGGTCCTTCTAACGACATACTGGATACAGCTGGAA |
| 1662 | NRAS 181 | Det Probe | AAGAAGAGTACAGTGCCATGAG |
| 1499 | NRAS 181C/182A (WT) | Template | TCTCTCATGGCACTGTACTCTTCTTGTCCAGCTGTATCCAGTATGTCCAACAAACAGGTTTCACCATCTA |
| 1500 | NRAS 181A (M) | Template | TCTCTCATGGCACTGTACTCTTCTTTTCCAGCTGTATCCAGTATGTCCAACAAACAGGTTTCACCATCTA |
| 1501 | NRAS 181/182 | Block. Probe 1 | ACATACTGGATACAGCTGGACA/T |
| 1502 | NRAS 181/182 | Block. Probe 2 | AGAAGAGTACAGTGCCATGAG |
| 1503 | KRAS 35G (WT) | Dir probe-c-s4 | GTGGCAACAGAAATCAGGTGGTGA CACTCTTGCCTACGCCAC |
| 1504 | KRAS 35A (M) | Dir probe-c-s9 | AGAGGACTGCTAAAGGTTTGTAGG CACTCTTGCCTACGCCAT |
| 1505 | KRAS 35 | Det Probe-c | CAGCTCCAACTACCACAAGTT |
| 1506 | KRAS 35G (WT) | Template-c | ACTGAATATAAACTTGTGGTAGTTGGAGCTGGTGGCGTAGGCAAGAGTGCCTTGACGATA |
| 1507 | KRAS 35A (M) | Template-c | ACTGAATATAAACTTGTGGTAGTTGGAGCTGATGGCGTAGGCAAGAGTGCCTTGACGATA |
| 1508 | KRAS 35 | Block. Probe 1c | CACTCTTGCCTACGCCAC/T |
| 1509 | KRAS 35 | Block. Probe 2c | CAGCTCCAACTACCACAAGTT |
| 1510 | PIK3CA 1633G (WT) | Dir probe-c-s3 | CTAATAGCTCCTGTGCCCTCGTAT CTCCATAGAAAATCTTTCTCCTGCTC |
| 1511 | PIK3CA 1633A (M) | Dir probe-c-s8 | AATCCGTCGACTAGCCTGAGAATT CTCCATAGAAAATCTTTCTCCTGCTT |
| 1512 | PIK3CA 1633 | Det Probe-c | AGTGATTTCAGAGAGAGGATCTCG |
| 1513 | PIK3CA 1633G (WT) | Template-c | AATTTCTACACGAGATCCTCTCTCTGAAATCACTGAGCAGGAGAAAGATTTTCTATGGAGTCACAGGTAAG |
| 1514 | PIK3CA 1633A (M) | Template-c | AATTTCTACACGAGATCCTCTCTCTGAAATCACTAAGCAGGAGAAAGATTTTCTATGGAGTCACAGGTAAG |
| 1515 | PIK3CA 1633 | Block. Probe 1c | CTCCATAGAAAATCTTTCTCCTGCTC/T |
| 1516 | PIK3CA 1633 | Block. Probe 2c | AGTGATTTCAGAGAGAGGATCTCG |
| 1517 | APC 4348C (WT) | Dir probe-c-s5 | TAGATGCCGCTGCAGGTATGGAAA GGTGCTTTATTTTTAGGTACTTCTCG |
| 1518 | APC 4348T (M) | Dir probe-c-s10 | CGTACCATTGAATCTGGAGACCTT GGTGCTTTATTTTTAGGTACTTCTCA |
| 1519 | APC 4348 | Det Probe-c | CTTGGTTTGAGCTGTTTGAGG |
| 1520 | APC 4348C (WT) | Template-c | TCCACCACCTCCTCAAACAGCTCAAACCAAGCGAGAAGTACCTAAAAAT AAAGCACCTACTGCTGAAAAG |
| 1521 | APC 4348T (M) | Template-c | TCCACCACCTCCTCAAACAGCTCAAACCAAGTGAGAAGTACCTAAAAAT AAAGCACCTACTGCTGAAAAG |
| 1522 | APC 4348 | Block. Probe 1c | GGTGCTTTATTTTTAGGTACTTCTCG/A |
| 1523 | APC 4348 | Block. Probe 2c | CTTGGTTTGAGCTGTTTGAGG |
| 1524 | NRAS 182A (WT) | Dir probe-s3 | CTAATAGCTCCTGTGCCCTCGTAT ACATACTGGATACAGCTGGACA |
| 1525 | NRAS 182T (M) | Dir probe-s8 | AATCCGTCGACTAGCCTGAGAATT ACATACTGGATACAGCTGGACT |
| 1526 | NRAS 182 | Det Probe | AGAAGAGTACAGTGCCATGAG |
| 1527 | NRAS 182T (M) | Template | TCTCTCATGGCACTGTACTCTTCTAGTCCAGCTGTATCCAGTATGTCCAA CAAACAGGTTTCACCATCTA |
| 1528 | NRAS 182A (WT) | Dir probe-s1 | ACTGGTAACCCAGACATGATCGGT ACATACTGGATACAGCTGGACA |
| 1529 | NRAS 182T (M) | Dir probe-s6 | ACTCCCTGTGGGTGAGCTTAATGG ACATACTGGATACAGCTGGACT |
| 1530 | TP53 524G (WT) | Dir probe-c-s2 | CATTTGGTCATTGGTTCAAGACGA TCATGGTGGGGGCAGC |
| 1531 | TP53 524A (M) | Dir probe-c-s 7 | CTGGTCCGTTGTGGTCCTTCTAAC TCATGGTGGGGGCAGT |
| 1532 | TP53 524 | Det Probe-c | GCCTCACAACCTCCGTCA |
| 1533 | TP53 524G (WT) | Template-c | CAGCACATGACGGAGGTTGTGAGGCGCTGCCCCCACCATGAGCGCTGCT CAGA |
| 1534 | TP53 524A (M) | Template-c | CAGCACATGACGGAGGTTGTGAGGCACTGCCCCCACCATGAGCGCTGCT CAGA |
| 1535 | TP53 524 | Block. Probe 1c | TCATGGTGGGGGCAGC/T |
| 1536 | TP53 524 | Block. Probe 2c | GCCTCACAACCTCCGTCA |

The OLA assay procedure includes the following steps:
1. Prepare OLA reaction mixture
   Combine the nucleic acid to be tested (e.g., genomic DNA, PCR-amplified DNA, whole genome amplified DNA, or a synthetic DNA analyte) with each directing probe, each detection probe and 500 U/mL Taq DNA ligase in Taq DNA ligase reaction buffer (New England Biolabs). Alternatively, HiFi Taq DNA ligase buffer (New England Biolabs) may be used to improve ligation specificity. For high target DNA levels (as in PCR products), the directing and detection probes are at 5 nM and 100 nM, respectively. For low target DNA levels, the probes are at 10 nM and 200 nM concentrations.
2. Run OLA reaction
   In a thermocycler, process the reaction mixture by (i) heating to 95° C. for 2 min., (ii) running 30 cycles of heating to 95° C. for 30 sec. then cooling to 62° C. for 5 min (for samples with low target levels of DNA) or 2 min. (for samples with high levels of target DNA) ., and (iii) heating to 95° C. for 5 min. Optionally, prior to the final heating condition, blocking probes that are complementary to (or include) the first and second probe sequences are at 50-fold excess relative to the OLA probes to prevent reformation of non-covalent complexes of the directing and detection probes.
3. Measure OLA reaction products by ECL assay
   The OLA reaction products are diluted to provide a solution with roughly the following levels of buffers and salts: 31% formamide, 400 mM NaCl, 1 mM EDTA, 0.01% Triton X-100 in 20 mM Tris-HCl, pH 8.0. This sample is analyzed to detect the reaction products as described in Example 3.

Optionally, the process can be repeated without adding ligase to determine the assay background signals in the absence of any ligated products. When measuring multiple alternative nucleotides at a SNP position, the percentage of each can be determined by comparing the specific signals from each. For example, when measuring the levels of a wild type and a mutant nucleotide at a SNP position, the specific signal for the wild type (WT) can be determined from signals measured on the array element capturing the wild type directing probe as SS_{WT} = S_{WT} -B_{WT}, where SS is specific signal, S is signal in the presence of ligase and B is the background in the absence of ligase. Similarly, the specific signal for the mutant (M) is determined from the array element capturing the mutant directing probe as SS_{M} = S_{M} - B_{M}. The percentage of nucleotides at the SNP position that are wild type or mutant are calculated as %WT = SS_{WT} / (SS_{WT} + SS_{M}) and %M = SS_{M} / (SS_{WT} + SS_{M}), respectively. These ratios can be used, for example, to analyze genomic DNA to identify heterozygosity. Possible %M thresholds for determining heterozygosity are %M < 0.2 (homozygous wild type), 0.3 < %M < 0.7 (heterozygous mutant) and 0.8 < %M (homozygous mutant). For many applications, the percentages may also be calculated using the signals (S) instead of the background corrected specific signals (SS).

### Example 8. Using OLA to Detect SNPs in Synthetic DNA Targets

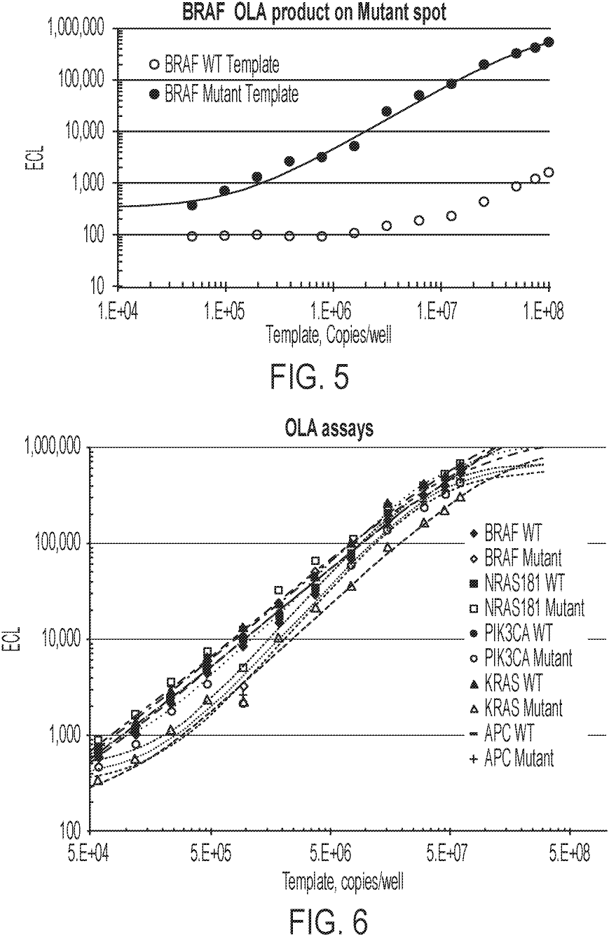

OLA assays were run for the detection of five mutations common in cancer, including melanoma and colon cancer: BRAF c.1799T>A (p.V600E); NRAS c.181C>A (p.Q61K); PIK3CA c.1633 G>A (p.E545K), KRAS c. 35G>A (p.G12D), and APC c.4348C>T (p.R1450*), where c.1799T>A represents the genetic mutation of nucleotide 1799 from T to A and p.V600E represents the resulting change in amino acid 600 of the coded protein from V to E. The OLA assays were run as described in Example 7 using the template sequences as the analyte and correspondent direct, detection and blocking probes (lines 1489-1523), with the use of the hot soak and blocking probes. FIG. 5 shows the signal from assay for the BRAF mutation (1799A) as a function of the number of template molecules (mutant or wild type) added per well and demonstrates that the assay has high specificity for the mutation relative to the wild type.

FIG. 6 shows the signal for all ten assays as a function of the number of template molecules and demonstrates that the assay signals increase linearly with template concentration. The limits of detection for the different assays were all around 2 × 10⁵ molecules per well. For each assay, Table 27 compares the signal measured for 10⁸ copies of the correct template vs. 10⁸ copies of the template with a single mis-match at the SNP site. The specificity, provided as the ratio of the signal for the matched vs. mis-matched sequence for 10⁸ template molecules, was greater than 100 for majority assays (ranging from 87 to 629 for WT>Mut substitution) indicating that the assays should be able to detect rare mutations at levels < 1% of the wild type.

TABLE 27

Specificity of OLA Assays

| Template (10⁸/Well) | WT BRAF 1799 | Mut BRAF 1799T>A | WT NRAS 181 | Mut NRAS 181C>A | WT PIK3CA 1633 | Mut PIK3CA 1633G>A | WT KRAS 35 | Mut KRAS 35G>A | WT APC 4348 | Mut APC 4348C>T |
|---|---|---|---|---|---|---|---|---|---|---|
| WT | 429,954 | 1,057 | 526,630 | 997 | 583,852 | 2,973 | 573,370 | 2,305 | 525,512 | 5,009 |
| Mutant | 1,615 | 426,351 | 843 | 626,968 | 1,353 | 342,822 | 954 | 269,483 | 17,717 | 433,408 |
| Specificity | 266 | 403 | 625 | 629 | 432 | 115 | 601 | 117 | 30 | 87 |

### Example 9. Use of Blocking Oligos for Reducing Non-specific Assay Background

OLA assay requires hybridization of probes to the analyte DNA (template) for the ligation to occur. The probes and template may stay hybridized even without a ligation event. This complex could bind to the capture oligo immobilized on the plate (via the directing probe) and generate signal (via detection probe) that is called here a bridging background. In cases of the low abundance of one allele over other (e.g., rare cancer mutations) the bridging background can be comparable to the signal originated from the ligation event on rare allele analyte such that bridging background could be misinterpreted as an actual specific signal leading to the false-positive results for the samples lacking the mutation.

One of the approaches for the mitigation of bridging background is a melting of DNA hybrids at high temperature (95C, and quick cool down to the 4C (or on ice). This procedure helps in the mitigation of bridging background but not to the extent required for the detection of low abundant mutations in the sample. In addition, this approach is hard to control thus small variations in the sample handling (how quick they are cooled after heating and handling during the loading to the plate) can potentially create conditions for the forming of non-desired complexes of DNA that affect background.

To evaluate the bridging background formation, the OLA samples were prepared for the 10-plex OLA assay (BRAF, NRAS181, PIK3CA, KRAS, and APC) as described in Example 8, with the exception that ligase was not added to the reaction mix. Synthetic templates at 10⁹ copies per reaction were used in the reaction mix, and ⅒^{th} of the reaction per assay well (10⁸ copies/well) was tested on plates as described in Example 7 in the presence and in the absence of blocking oligos.

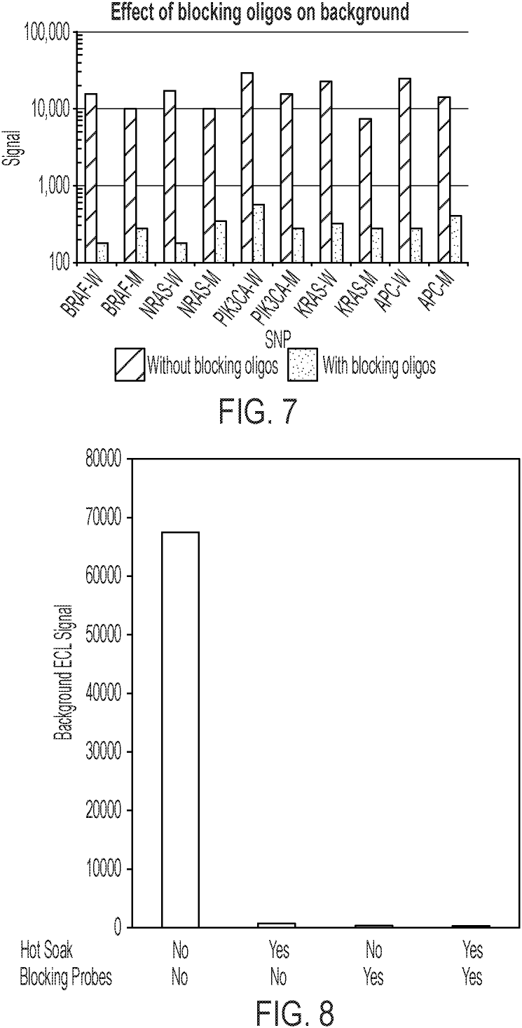

As shown on FIG. 7, backgrounds ranged between 7,000 and 30,000 counts for the samples tested without blocking oligos, presumably due to some level of non-covalent attachment of directing probes to detection probes through rehybridization to residual template (“bridiging”). In the presence of blocking oligos the background signal for the same samples drops significantly to 180 to 550 counts. In the absence of blocking oligos, bridging background increases with a template concentration increase (data not shown) becoming most significant at high template concentrations (e.g., 10⁸ copies per well and higher). Blocking probes are, therefore, most useful for experimental conditions with high template concentration (e.g., the detection of rare cancer mutations in a high background of wildtype sequences).

### Example 10. Effect of Blocking Oligos on OLA Sensitivity and Specificity

To evaluate the effect of blocking oligos on assay sensitivity and specificity, three OLA assays were tested in the presence and in the absence of blocking oligos: BRAF c. 1799T>A (p.V600E); NRAS c. 181C>A (p.Q61K); and NRAS c. 182A>T (p.Q61L). OLA samples were prepared using synthetic templates and OLA probes for BRAF and NRAS181 assays described in Example 8, and for NRAS182 assay using sequences listed in Example 7 (line 1524-27). OLA samples were tested as described in the Example 7; each sample was tested with and without addition of blocking oligos prior to the final heating step. For each assay, Table 28 compares the signal measured for 2×10⁸ copies of the correct template vs. 2×10⁸ copies of the template with a single mis-match at the SNP site with and without blocking oligos added to the sample before heating and loading to the plate. The table shows that the addition of blocking oligos had only marginal effects on specific signals (i.e., the signal for a target on the correct spot), showing that the blocking oligos did not reduce assay sensitivity. The table also shows that the non-specific signals on the incorrect spots were significantly reduced, leading to an improvement in specificity. The specificity, provided as the ratio of the signal for the matched vs. mis-matched sequence for 10⁸ template molecules, was improved up to 15-fold when blocking oligos were added to the sample. Specificity improvement was calculated by dividing Specificity in the presence of blocking oligo to the Specificity in the absence of blocking oligo.

TABLE 28

Specificity of OLA Assays tested in the presence and the absence of blocking oligos

| Blocking oligo | Template (2×10⁸ / Well) | WT BRAF 1799 | Mutant BRAF 1799T>A | WT NRAS 181 | Mutant NRAS 181C>A | WT NRAS 182 | Mutant NRAS 182A>T |
|---|---|---|---|---|---|---|---|
| Added | WT1 | 314,661 | 476 | 316,076 | 572 | 296,272 | 604 |
| | Mutant | 3,429 | 253,048 | 1,339 | 337,992 | 275 | 316,291 |
| | Specificity | 92 | 532 | 236 | 591 | 1,077 | 524 |
| Not Added | WT1 | 266,818 | 7,279 | 305,738 | 4,998 | 238,243 | 2,445 |
| | Mutant | 9,238 | 256,789 | 4,674 | 320,812 | 3,382 | 291,775 |
| | Specificity | 29 | 35 | 65 | 64 | 70 | 119 |
| Specificity improvement | | 3 | 15 | 4 | 9 | 15 | 4 |

### Example 11. Use of Hot Soak or Blocking Probes to Reduce Non-Specific Background in OLA Format

The procedure for capturing and measuring biotin-labeled oligonucleotides in Example 3 carries out hybridization under stringent conditions (including elevated temperature) to minimize non-specific hybridization reaction. The cooling of the plate after hybridization and prior to the plate wash, however, provides some time under less stringent conditions where it is possible for some non-specific hybridization reaction to occur which may persist through the wash step. One approach to mitigate this effect is to cool quickly to 4° C. (or on ice) to slow the kinetics of the non-specific hybridization, but the timing for cooling a plate may be difficult to control. Therefore, two other approaches were developed that individually, or in tandem, were found to greatly reduce observed non-specific hybridization: the use of blocking probes and the use of a hot soak step.

A 10-plex OLA assay was developed using the plates described in Example 2 for measuring the wild type and mutant forms of five different SNPs: NRAS c. 182A>T, TP53 c.524G>A, PIK3CA c.1633G>A, KRAS c.35G>A, and APC c.4348C>T. OLA samples were prepared using synthetic templates and OLA probes for PIK3C, KRAS, and APC as described in Example 8 with additional sequences for NRAS 182 and TP53 from the sequence table in Example 7 (line 1526-36). In this assay, the biotin-labeled detection probe for the KRAS SNP assays was found to have a weak interaction with the capture oligonucleotide on spot 6 of the capture oligonucleotide array that leads to elevated background signals on that spot in the absence of ligation. FIG. 8 shows the elevated background signal that was observed for spot 6 when the assay was run as described in Example 7, but in the absence of analyte or ligase, and without the use of blocking oligonucleotides or the hot soak step.

The blocking oligonucleotides were added (at 50-fold excess relative to the OLA probes) on completion of the ligation step during the OLA protocol, but before the final 95° C. denaturation step. FIG. 8 shows that addition of the blocking probes drastically reduced the level of non-specific binding.

The hot soak step is carried out after the incubation of the OLA products to the capture oligonucleotide array and washing of the array to remove excess unbound reagents. The hot soak that was employed was an additional 30 min. incubation under stringent conditions -low salt (0.1X PBS) and elevated temperature (37° C.) - that allowed weakly bound nucleotides to be dissociated and then washed away. FIG. 8 shows that the hot soak step, like the blocking probes, drastically reduced the level of non-specific binding. Even further reductions could be achieved by employing both the blocking probes and the hot soak step. The blocking probes and hot soak step did not have a significant effect on true signal from OLA products (data not shown).

### Example 12. Use of OLA to Detect Mutations in Whole Genome Amplification (WGA) Products and Genomic DNA Without Amplification

Cell lines heterozygous with respect to the mutation in either BRAF or NRAS gene were selected from ATCC collections as shown in Table 29.

TABLE 29

ATCC Cell Line Genomic DNA

| ATCC Cell Line Genomic DNA | Genotype | Percent Mutant |
|---|---|---|
| A2058 (Melanoma) | BRAF c. 1799T>A | 50% |
| NCI-H1299 (non-small cell lung cancer) | NRAS c. 181C>A | 50% |
| HL-60 (acute promyelocytic leukemia) | NRAS c. 182A>T | 50% |

DNA from cell lines A2058 and NCI-H1299 was subjected to whole genome amplification (WGA) using a REPLI-g Amplification kit (QIAGEN), 10 ng/reaction; DNA from cell line HL-60 was used in OLA reaction without amplification. OLA assays were performed as described in Example 8, above. Two WGA DNA samples were tested with BRAF, NRAS181, PIK3CA, KRAS and APC assays, HL60 gDNA was tested with BRAF, TP53, PIK3CA KRAS and NRAS182 assays. 5-15 ug of DNA sample was used in OLA reaction and 2-6 ug went into the ECL assay well.

For each assay conducted on each sample, Tables 30-32 present the % of the measured sequences that had the target mutation (calculated as described in Example 7). Measured mutation percentages < 20% were classified as homozygous wildtype samples, mutation percentages between 30% and 70% were classified as heterozygous (50% mutation) and mutation percentages above 80% were classified as homozygous mutants. The tables show that each cell line was correctly classified based on its expected genotype.

TABLE 30

OLA results with WGA DNA from cell line A2058 (BRAF 1799T>A heterozygous)

| | Assay Result | Heterozygosity | |
|---|---|---|---|
| Assay | (% Mutation) | Expected | Measured |
| BRAF 1799T>A | 35.1 | Heterozygous | Heterozygous |
| NRAS c. 181C>A | 2.0 | Homozygous (WT) | Homozygous (WT) |
| PIK3CA c. 1633G>A | 4.9 | Homozygous (WT) | Homozygous (WT) |
| KRAS c.35G>A | 4.1 | Homozygous (WT) | Homozygous (WT) |
| APC c.4348C>T | 4.0 | Homozygous (WT) | Homozygous (WT) |

TABLE 31

OLA results with WGA DNA from cell line NCI-H1299 (NRAS 181C>A heterozygous)

| | Assay Result | Heterozygosity | |
|---|---|---|---|
| Assay | (% Mutation) | Expected | Measured |
| BRAF 1799T>A | 2.0 | Homozygous (WT) | Homozygous (WT) |
| NRAS c. 181C>A | 46.0 | Heterozygous | Heterozygous |
| PIK3CA c. 1633G>A | 4.2 | Homozygous (WT) | Homozygous (WT) |
| KRAS c.35G>A | 3.4 | Homozygous (WT) | Homozygous (WT) |
| APC c.4348C>T | 4.2 | Homozygous (WT) | Homozygous (WT) |

TABLE 32

OLA results with gDNA from cell line HL-60 (NRAS 182A>T heterozygous)

| | Assay Result | Heterozygosity | |
|---|---|---|---|
| Assay | (% Mutation) | Expected | Measured |
| BRAF 1799T>A | 0.3 | Homozygous (WT) | Homozygous (WT) |
| TP53 c.524G>A | -0.4 | Homozygous (WT) | Homozygous (WT) |
| PIK3CA c. 1633G>A | 1.2 | Homozygous (WT) | Homozygous (WT) |
| KRAS c.35G>A | 4.7 | Homozygous (WT) | Homozygous (WT) |
| NRAS c.182A>T | 57.4 | Heterozygous | Heterozygous |

### Example 13. Use of OLA to Detect PCR Products

To create mock cancer samples for BRAF c.1799T>A and NRAS c. 181C>A mutations that mimic low levels of a mutation in a wild type background, genomic DNA from different ATCC cell lines (Table 29) was mixed at pre-specified levels to create mutant levels ranging from 0 to 50%. As shown in the table, each cell line was heterozygous for one of three mutations that are commonly seen in melanomas: BRAF c. 1799T>A (p.V600E); NRAS c. 181C>A (p.Q61K). To create BRAF c.1799T>A samples, genomic DNA from cell lines A2058 and NCI-H1299 were mixed. To create NRAS c. 181C>A samples, genomic DNA from cell lines A2058 and NCI-H1299 were mixed. NRAS and BRAF amplicons were generated by polymerase chain reaction (PCR) (35 cycles, 10 ng of genomic DNA input) for each mock cancer sample.

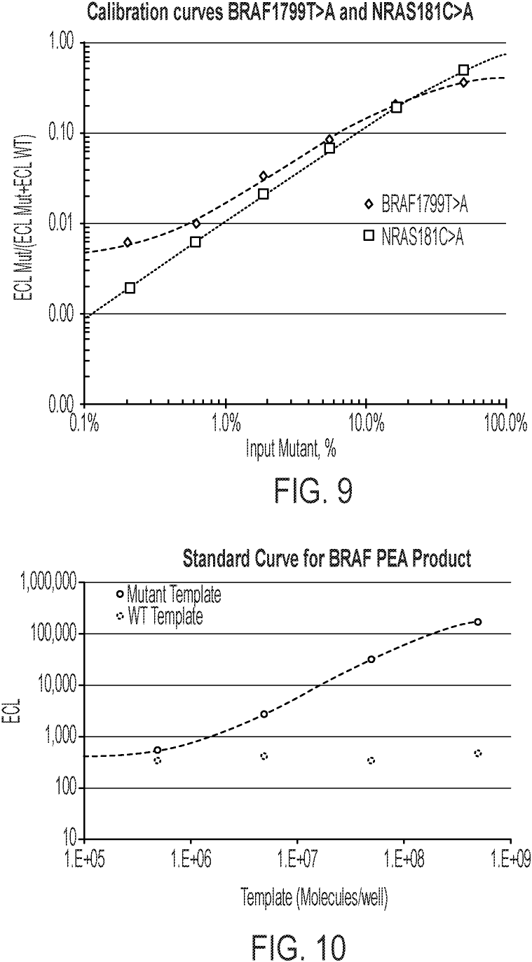

Oligonucleotide ligation assays for the mutant and wild type SNPs were performed on the PCR amplified samples as described in Examples 8. PCR product was diluted and 0.01 ul used per 20 ul OLA mix; ⅒^{th} of OLA product per assay well was tested on plates (0.001 ul of PCR product/assay well). The results were used to calculate the percent of each SNP with the mutant nucleotide. The calculated percentage as a function of the predicted percentage of mutant nucleotide (based on the mixture of cell line DNA) is provided in Tables 33 and 34, and in FIG. 9. The figures and tables show that the calculated ratios closely approximate the predicted ratios, and also show that in almost all assays a mutation rate as low as 0.2% could be differentiated from a pure wild type sample.

TABLE 33

BRAF 1799T>A mutation: OLA results with PCR amplified genomic DNA
BRAF 1799T>A Mutant; Mixed Genomic DNA; OLA results
Fitted with Cal Curve

| Input mutant, % | ECL Ratio | % calculated |
|---|---|---|
| 50.0 | 0.37 | 55.81 |
| 16.7 | 0.20 | 15.41 |
| 5.6 | 0.09 | 5.53 |
| 1.9 | 0.03 | 2.10 |
| 0.6 | 0.01 | 0.54 |
| 0.2 | 0.006 | 0.23 |
| 0 | 0.002 | ND |

TABLE 34

NRAS 181C>A mutation: OLA results with PCR amplified genomic DNA
NRAS 181C>A Mutant; Mixed Genomic DNA; OLA results
Fitted with Cal Curve

| Input mutant, % | ECL Ratio | % calculated |
|---|---|---|
| 50.0 | 0.490 | 50.46 |
| 16.7 | 0.189 | 16.26 |
| 5.6 | 0.066 | 5.67 |
| 1.9 | 0.020 | 1.87 |
| 0.6 | 0.006 | 0.61 |
| 0.2 | 0.002 | 0.21 |
| 0 | 0.001 | ND |

### Example 14. A Procedure for Polymerase Extension Assay (PEA) to Detect Single Nucleotide Polymorphisms (SNPs)

Detection of a SNP is carried out using an oligonucleotide probe a shown in FIGS. 2A 2B: a directing probe that includes a sequence towards the 5′ end that includes a sequence selected from SEQ ID NOs: 745 to 754 (i.e., a sequence that hybridizes to one of the capture oligonucleotides in the arrays prepared as described in Example 2) and a first probe sequence at the 3′ end that is complementary to the analyte nucleic acid sequence downstream from the SNP site (such that the 3′ end is complementary to the nucleotide one position downstream from the SNP nucleotide in the analyte). In the presence of the analyte, a polymerase and the complementary biotin-modified dideoxy nucleoside triphosphate (ddNTP) to the SNP site, the directing probe is extended to include the biotin-modified nucleotide. When comparing the levels of different nucleotides at a SNP position (e.g., the levels of a wild type nucleotide vs. a mutant nucleotide), the reaction is repeated in different wells with the appropriate ddNTPs for the nucleotide at the SNP position.

The PEA assay procedure includes the following steps:
1. Prepare PEA reaction mixture
   Combine the nucleic acid to be tested (e.g., genomic DNA, PCR-amplified DNA, whole genome amplified DNA, or a synthetic DNA analyte) with the 50 nM directing probe, 2 uM each biotin-ddNTP complementary to the SNP nucleotide of interest and unlabeled ddNTPs and 120 U/mL of Therminator™ DNA Polymerase in ThermoPol® Reaction buffer
2. Run PEA reaction
   In a thermocycler, process the reaction mixture by (i) heating to 96° C. for 2 min., (ii) running 30 cycles of heating to 95° C. for 30 sec. then cooling to 55° C. for 30 sec and heating to 72° C. for 30 sec.
3. Measure PEA reaction products by ECL assay
   The PEA reaction products are diluted to provide a solution with roughly the following levels of buffers and salts: 31% formamide, 400 mM NaCl, 1 mM EDTA, 0.01% Triton X-100 in 20 mM Tris-HCl, pH 8.0. This sample is analyzed to detect the reaction products as described in Example 3.

Measurements can optionally be repeated in the absence of polymerase to determine the background signals in the absence of extended probes. As described for the OLA format in Example 7, comparison of the signals or background-corrected specific signals for assays detecting different nucleotides at a given SNP position can be used to estimate the percentage of the nucleic acids in the sample with each nucleotide.

### Example 15. Using PEA to Detect SNPs in Synthetic DNA Targets

Oligonucleotide probes of the primer extension assay (PEA) were designed for the detection of three mutations common in melanoma: BRAF c. 1799T>A (p.V600E); NRAS c. 181C>A (p.Q61K); and NRAS c. 182A>T (p.Q61L). A directing probe was created for each SNP position of interest. An early prototype capture array was used relative to the array in previous examples. The directing probes are listed below in Table 35 with regions complementary to the capture oligonucleotides shown in caps:

TABLE 35

Directing probes

| SEQ ID NO | SNP | Reagent | Sequence |
|---|---|---|---|
| 1537 | BRAF 1799 | Dir. Probe | GCTCCCGTTAATGCTCCCGTTAAT aggtgattttggtctagctacag |
| 1538 | NRAS 181 | Dir. Probe | TAGCAAGGGAAATAGCAAGGGAAA gacatactggatacagctgga |
| 1539 | NRAS 182 | Dir. Probe | TGGTGAATTAGCTGGTGAATTAGC acatactggatacagctggac |

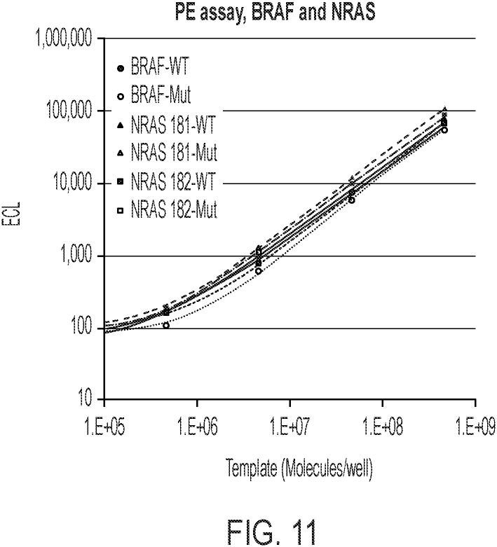

Assays were run as described in Examples 14 and 3 using the template sequences as the analyte, except for the use of different capture sequences. In this experiment the hot soak step was not used. FIG. 10 shows the signal from assay for the BRAF mutation (1799A) as a function of the number of template molecules (mutant or wild type) added per well and demonstrates that the assay has high specificity for the mutation relative to the wild type. FIG. 11 shows the signal for all six assays as a function of the number of template molecules (using the correct template for each assay) and demonstrates that the assay signals increase linearly with template concentration. The limits of detection for the different assays were all around 5 × 10⁵ molecules per well. For each assay, Table 36 compares the signal measured for 10⁸ copies of the correct template vs. 10⁸ copies of the template with a single mis-match at the SNP site. The specificity, provided as the ratio of the signal for the matched vs. mis-matched sequence for 10⁸ template molecules, ranged from roughly 10² to 10³ indicating that the assays should be able to detect rare mutations at levels < 1% of the wild type.

TABLE 36

Specificity of PEA Assays

| Template (4.8×10⁸ / Well) | WT BRAF 1799 | Mutant BRAF 1799T>A | WT NRAS 181 | Mutant NRAS 181C>A | WT NRAS 182 | Mutant NRAS 182A>T |
|---|---|---|---|---|---|---|
| WT1 | 72,196 | 88 | 36,977 | 1,496 | 51,391 | 86 |
| Mutant | 123 | 54,236 | 117 | 102,837 | 419 | 77,574 |
| Specificity | 587 | 616 | 316 | 69 | 123 | 902 |

### Example 16. Use of PEA to Detect PCR Products

NRAS and BRAF amplicons were generated by polymerase chain reaction (PCR) (35 cycles, 60 ng of genomic DNA input) using genomic DNA extracted from the ATCC cell lines shown in Table 29. As shown in the table, each cell line was heterozygous for one of three mutations commonly seen in melanomas: BRAF c.1799T>A (p.V600E); NRAS c. 181C>A (p.Q61K); or NRAS c. 182A>T (p.Q61L). To create mock cancer samples that mimic low levels of a mutation in a wild type background, the cell line DNA was mixed at pre-specified levels to create mutant levels ranging from 0 to 50%. To create BRAF c. 1799T>A samples, genomic DNA from cell lines A2058 and NCI-H1299 were mixed. To create NRAS c. 181C>A samples, genomic DNA from cell lines A2058 and NCI-H1299 were mixed. To create NRAS c. 182A>T samples, genomic DNA from cell lines A2058 and HL-60 were mixed.

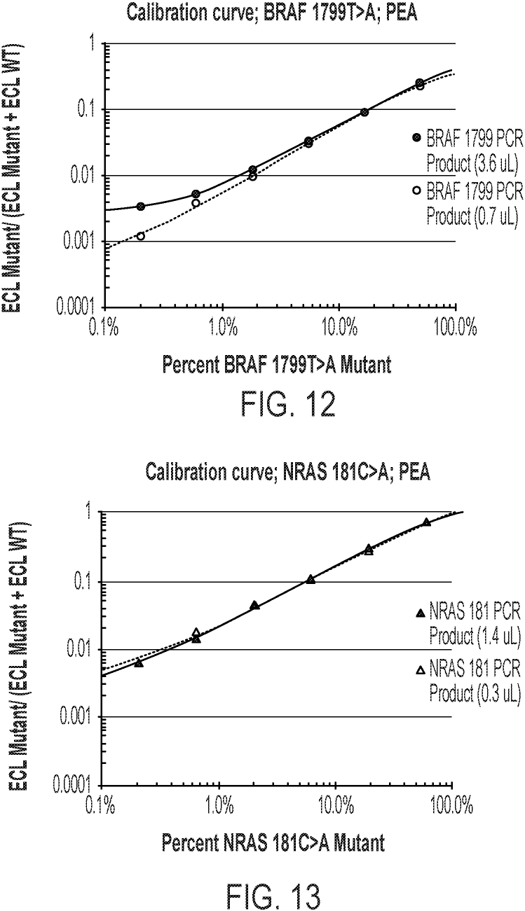

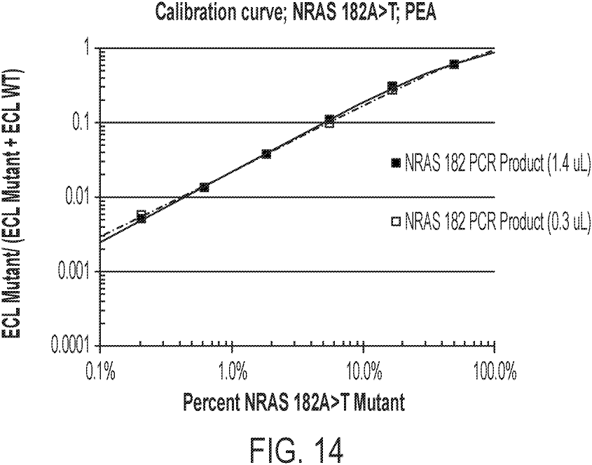

Primer extension assays for the mutant and wild type SNPs were performed on the samples as described in Example 15. For each sample, two different dilutions of PCR product were tested. The results were used to calculate the percent of each SNP with the mutant nucleotide. The calculated percentage as a function of the predicted percentage of mutant nucleotide (based on the mixture of cell line DNA) is provided in table and graphical format: BRAF 1799T>A results (Table 37 and FIG. 12); NRAS 181C>8 results (Table 38 and FIG. 13); NRAS 182A>T results (Table 39, FIG. 14). The figures and tables show that the calculated ratios closely approximate the predicted ratios, and also show that in almost all assays a mutation rate as low as 0.2% could be differentiated from a pure wild type sample.

TABLE 37

BRAF 1799T>A mutation: PEA results with genomic DNA
BRAF 1799T>A Mutant; Mixed Genomic DNA; PEA results

| Fitted with Cal Curve | | 0.04 µl PCR product | 0.0008 µl PCR product |
|---|---|---|---|
| Input mutant | ECL Ratio | % calculated | |
| 50.0% | 0.253 | 50.4% | 57.2% |
| 16.7% | 0.092 | 16.2% | 16.6% |
| 5.6% | 0.033 | 5.7% | 6.0% |
| 1.9% | 0.012 | 1.9% | 2.3% |
| 0.6% | 0.005 | 0.6% | 1.0% |
| 0.2% | 0.003 | 0.2% | 0.6% |
| 0% | 0.002 | 0.0% | 0.5% |

TABLE 38

NRAS 181C>A mutation: PEA results with genomic DNA
NRAS 181C>A Mutant; Mixed Genomic DNA; PEA results

| Fitted with Cal Curve | | 0.04 µl PCR product | 0.0008 µl PCR product |
|---|---|---|---|
| Input mutant | ECL Ratio | % calculated | |
| 50.0% | 0.686 | 50.4% | 50.8% |
| 16.7% | 0.298 | 16.6% | 18.7% |
| 5.6% | 0.107 | 5.3% | 5.8% |
| 1.9% | 0.044 | 2.1% | 2.1% |
| 0.6% | 0.014 | 0.6% | 0.5% |
| 0.2% | 0.006 | 0.2% | 0.2% |
| 0% | 0.002 | 0.0% | ND |

TABLE 39

NRAS 182A>T mutation: PEA results with genomic DNA
NRAS 182A>T Mutant; Mixed Genomic DNA; PEA results

| Fitted with Cal Curve | | 0.04 µl PCR product | 0.0008 µl PCR product |
|---|---|---|---|
| Input mutant | ECL Ratio | % calculated | |
| 50.0% | 0.609 | 48.2% | 49.8% |
| 16.7% | 0.303 | 17.9% | 20.0% |
| 5.6% | 0.113 | 5.7% | 6.3% |
| 1.9% | 0.037 | 1.7% | 1.8% |
| 0.6% | 0.013 | 0.6% | 0.6% |
| 0.2% | 0.005 | 0.2% | 0.2% |
| 0% | 0.000 | ND | ND |

### Example 17. Oligonucleotide Ligation Assay (OLA) to Detect Cystic Fibrosis (CF) Mutations

Cystic Fibrosis (CF) is caused by a mutation in the cystic fibrosis transmembrane conductance regulator (CFTR) gene. The most common mutation, ΔF508, is a deletion (Δ signifying deletion) of three nucleotides that results in a loss of the amino acid phenylalanine (F) at the 508th position on the protein. The ΔF508 mutation accounts for two-thirds (66-70%) of CF cases worldwide and 90% of cases in the United States. The ΔF508 mutation has its highest rates in people of Northern European descent. The next most common mutation is the G542X mutation, which accounts for about 5% of the CF cases in the United States.

The CF mutations can be detected using the method described in Example 7. OLA probes for the detection of CF mutations are listed in Table 40. The regions that are complementary to the capture oligonucleotides are shown in bold.

TABLE 40

OLA probe sequences for CF testing

| SEQ ID NO: | Site | Reagent | Sequence |
|---|---|---|---|
| 1540 | CF Δ508 (WT) | Dir probe-s1 | ACTGGTAACCCAGACATGATCGGT-CTGGCACCATTAAAGAAAATATCATCTT |
| 1541 | CF Δ508 (M) | Dir probe-s6 | ACTCCCTGTGGGTGAGCTTAATGG-GCCTGGCACCATTAAAGAAAATATCAT |
| 1542 | CF Δ508 | Det Probe | pTGGTGTTTCCTATGATGAATATAGATACAG-Biotin |
| 1543 | CF G542X, C>A (WT) | Dir probe-s3 | CTAATAGCTCCTGTGCCCTCGTAT-ACTCAGTGTGATTCCACCTTCTCC |
| 1544 | CF G542X C>A (M) | Dir probe-s8 | AATCCGTCGACTAGCCTGAGAATT-ACTCAGTGTGATTCCACCTTCTCA |
| 1545 | CF G542X | Det Probe | pAAGAACTATATTGTCTTTCTCTGCAAAC-Biotin |

### Example 18. Oligonucleotide Ligation Assay (OLA) to Detect BRCA Mutations

BRCA mutations are germline mutation in either the BRCA1 or BRCA2 genes, which are tumor suppressor genes. Mutations in these genes may produce a hereditary breast-ovarian cancer syndrome in affected persons. Common mutations in these genes include BRCA1*185delAG (exon 2), BRCA1*5382insC (exon 20) and BRCA2*6174delT (exon 11).

BRCA mutations can be detected using the method described in Example 7. OLA probes for the detection of BRCA mutations are listed in Table 41. The regions that are complementary to the capture oligonucleotides shown in bold.

TABLE 41

OLA probe sequences for BRCA testing

| SEQ ID NO: | Site | Reagent | Sequence |
|---|---|---|---|
| 1546 | BRCA1*185delAG (WT) | Dir probe-s2 | CATTTGGTCATTGGTTCAAGACGA-TTAATGCTATGCAGAAAATCTTAGAG |
| 1547 | BRCA1*185delAG (M) | Dir probe-s7 | CTGGTCCGTTGTGGTCCTTCTAAC-TCATTAATGCTATGCAGAAAATCTTAG |
| 1548 | BRCA1*185delAG | Det Probe | pTGTCCCATCTGTCTGGAGTTGA-Biotin |
| 1549 | BRCA1*5382insC (WT) | Dir probe-s3 | CTAATAGCTCCTGTGCCCTCGTAT-CAAAGCGAGCAAGAGAATCCC |
| 1550 | BRCA1*5382insC (M) | Dir probe-s8 | AATCCGTCGACTAGCCTGAGAATT-AAAGCGAGCAAGAGAATCCCC |
| 1551 | BRCA1*5382insC | Det Probe | pAGGACAGAAAGATCTTCAGGGGG-Biotin |
| 1552 | BRCA2*6174delT (WT) | Dir probe-s1 | ACTGGTAACCCAGACATGATCGGT-GTGGGATTTTTAGCACAGCAAGT |
| 1553 | BRCA2*6174delT (M) | Dir probe-s6 | ACTCCCTGTGGGTGAGCTTAATGG-TGTGGGATTTTTAGCACAGCAAG |
| 1554 | BRCA2*6174delT | Det Probe | pGGAAAATCTGTCCAGGTATCAGATG-Biotin |

### Example 19. Lung Cancer SNP Panel

A panel of 9 SNPs that are associated with an enhanced risk for lung cancer development was developed. These mutations were:
rs1801133 (MTHFR C677T)
rs1801270 (CDKN1A c.93C>A)
rs3842 (ABCB1 c.*193A>G)
rs1051730 (CHRNA3 D398N)
rs8034191 (LOC123688 or HYKK c.337+256T>C)
rs212090 (ABCC1 c.*866T>A)
rs2273535 (AURKA F31I)
rs17879961 (CHEK2 c.599T>C)
rs2243828 (MPO c.-764T>C)

The probe sequences used for each mutation are provided in Tables 42 (upstream) and 43 (downstream), below.

TABLE 42

Upstream Probes

| SNP | Strand | Upstream Probe | SEQ ID NO: | Spot |
|---|---|---|---|---|
| MTHFR rs180113 WT | 1^{st} strand | ACTGGTAACCCAGACATGATCGGT AGAAGGTGTCTGCGGGAGC | 1555 | 1 |
| | 2^{nd} strand | ACTGGTAACCCAGACATGATCGGT GCTGCGTGATGATGAAATCGG | 1556 | |
| MTHFR rs180113 MUT | 1^{st} strand | ACTCCCTGTGGGTGAGCTTAATGG AGAAGGTGTCTGCGGGAGT | 1557 | 6 |
| | 2^{nd} strand | ACTCCCTGTGGGTGAGCTTAATGG GCTGCGTGATGATGAAATCGA | 1558 | |
| CDKN1A rs1801270 WT | 1^{st} strand | CATTTGGTCATTGGTTCAAGACGA GACAGCGAGCAGCTGAGC | 1559 | 2 |
| | 2^{nd} strand | CATTTGGTCATTGGTTCAAGACGA GCGCATCACAGTCGCGG | 1560 | |
| CDKN1A rs1801270 MUT | 1^{st} strand | CTGGTCCGTTGTGGTCCTTCTAAC GACAGCGAGCAGCTGAGA | 1561 | 7 |
| | 2^{nd} strand | CTGGTCCGTTGTGGTCCTTCTAAC GCGCATCACAGTCGCGT | 1562 | |
| ABCB1 rs3842 WT | 1^{st} strand | CTAATAGCTCCTGTGCCCTCGTAT GAGACATCATCAAGTGGAGAGAAATCA | 1563 | 3 |
| | 2^{nd} strand | CTAATAGCTCCTGTGCCCTCGTAT CTGTTATAAAATTTATAATGCAGTTTAAACTAT | 1564 | |
| ABCB1 rs3842 MUT | 1^{st} strand | AATCCGTCGACTAGCCTGAGAATT GAGACATCATCAAGTGGAGAGAAATCG | 1565 | 8 |
| | 2^{nd} strand | AATCCGTCGACTAGCCTGAGAATT CTGTTATAAAATTTATAATGCAGTTTAAACTAC | 1566 | |
| CHRNA3 rs1051730 WT | 1^{st} strand | GTGGCAACAGAAATCAGGTGGTGA CATCATCAAAGCCCCAGGCTAC | 1567 | 4 |
| | 2^{nd} strand | GTGGCAACAGAAATCAGGTGGTGA AGTTGTACTTGATGTCGTGTTTG | 1568 | |
| CHRNA3 rs1051730 MUT | 1^{st} strand | AGAGGACTGCTAAAGGTTTGTAGG CATCATCAAAGCCCCAGGCTAT | 1569 | 9 |
| | 2^{nd} strand | AGAGGACTGCTAAAGGTTTGTAGG AGTTGTACTTGATGTCGTGTTTA | 1570 | |
| LOC1236 88 rs8034191 WT | 1^{st} strand | TAGATGCCGCTGCAGGTATGGAAA CCAATGTGGTATAAGTTTTCTGTTT | 1571 | 5 |
| | 2^{nd} strand | TAGATGCCGCTGCAGGTATGGAAA TTACTATCTGTCAGGGCCTTTCTA | 1572 | |
| LOC1236 88 rs8034191 MUT | 1^{st} strand | CGTACCATTGAATCTGGAGACCTT CCAATGTGGTATAAGTTTTCTGTTC | 1573 | 10 |
| | 2^{nd} strand | CGTACCATTGAATCTGGAGACCTT TTACTATCTGTCAGGGCCTTTCTG | 1574 | |
| ABCC1 rs212090 WT | 1^{st} strand | ACTGGTAACCCAGACATGATCGGT AGAACAATCAATGCTGTTATTACTGT | 1575 | 1 |
| | 2^{nd} strand | ACTGGTAACCCAGACATGATCGGT CCACATCAATCATGGTGGGAA | 1576 | |
| ABCC1 rs212090 MUT | 1^{st} strand | ACTCCCTGTGGGTGAGCTTAATGG AGAACAATCAATGCTGTTATTACTGA | 1577 | 6 |
| | 2^{nd} strand | ACTCCCTGTGGGTGAGCTTAATGG CCACATCAATCATGGTGGGAT | 1578 | |
| AURKA rs2273535 WT | 1^{st} strand | CTAATAGCTCCTGTGCCCTCGTAT CCAAAACGTGTTCTCGTGACTCAGCAAT | 1579 | 3 |
| | 2^{nd} strand | CTAATAGCTCCTGTGCCCTCGTAT TACAGGTAATGGATTCTGACAAGGAAA | 1580 | |
| AURKA rs2273535 MUT | 1^{st} strand | AATCCGTCGACTAGCCTGAGAATT CCAAAACGTGTTCTCGTGACTCAGCAAA | 1581 | 8 |
| | 2^{nd} strand | AATCCGTCGACTAGCCTGAGAATT TACAGGTAATGGATTCTGACAAGGAAT | 1582 | |
| CHEK2 rs1787996 1 WT | 1^{st} strand | GTGGCAACAGAAATCAGGTGGTGA AGTGGGTCCTAAAAACTCTTACAT | 1583 | 4 |
| | 2^{nd} strand | GTGGCAACAGAAATCAGGTGGTGA CCACTGTGATCTTCTATGTATGCAA | 1584 | |
| CHEK2 rs1787996 1 MUT | 1^{st} strand | AGAGGACTGCTAAAGGTTTGTAGG AGTGGGTCCTAAAAACTCTTACAC | 1585 | 9 |
| | 2^{nd} strand | AGAGGACTGCTAAAGGTTTGTAGG CCACTGTGATCTTCTATGTATGCAG | 1586 | |
| MPO rs2243828 WT | 1^{st} strand | TAGATGCCGCTGCAGGTATGGAAA CACCATTGTGTGCCTATACCA | 1587 | 5 |
| | 2^{nd} strand | TAGATGCCGCTGCAGGTATGGAAA CCCTGGGGACAAGCACT | 1588 | |
| MPO rs2243828 MUT | 1^{st} strand | CGTACCATTGAATCTGGAGACCTT CACCATTGTGTGCCTATACCG | 1589 | 10 |
| | 2^{nd} strand | CGTACCATTGAATCTGGAGACCTT CCCTGGGGACAAGCACC | 1590 | |

TABLE 43

Downstream Probes

| SNP | Strand | Downstream Probe | SEQ ID NO: | Spot |
|---|---|---|---|---|
| MTHFR rs180113 WT | 1^{st} strand | /5Phos/CGATTTCATCATCACGCAGC/3Bio/ | 1591 | 1 |
| | 2^{nd} strand | /5Phos/CTCCCGCAGACACCTTCTC/3Bio/ | 1592 | |
| MTHFR rs180113 MUT | 1^{st} strand | /5Phos/CGATTTCATCATCACGCAGC/3Bio/ | 1593 | 6 |
| | 2^{nd} strand | /5Phos/CTCCCGCAGACACCTTCTC/3Bio/ | 1594 | |
| CDKN1A rs1801270 WT | 1^{st} strand | /5Phos/CGCGACTGTGATGCGCTA/3Bio/ | 1595 | 2 |
| | 2^{nd} strand | /5Phos/CTCAGCTGCTCGCTGTCC/3Bio/ | 1596 | |
| CDKN1A rs1801270 MUT | 1^{st} strand | /5Phos/CGCGACTGTGATGCGCTA/3Bio/ | 1597 | 7 |
| | 2^{nd} strand | /5Phos/CTCAGCTGCTCGCTGTCC/3Bio/ | 1598 | |
| ABCB1 rs3842 WT | 1^{st} strand | /5Phos/TAGTTTAAACTGCATTATAAATTTTATAACAG/3Bio/ | 1599 | 3 |
| | 2^{nd} strand | /5Phos/GATTTCTCTCCACTTGATGATGTCTC/3Bio/ | 1600 | |
| ABCB1 rs3842 MUT | 1^{st} strand | /5Phos/TAGTTTAAACTGCATTATAAATTTTATAACAG/3Bio/ | 1601 | 8 |
| | 2^{nd} strand | /5Phos/GATTTCTCTCCACTTGATGATGTCTC/3Bio/ | 1602 | |
| CHRNA3 rs1051730 WT | 1^{st} strand | /5Phos/AAACACGACATCAAGTACAACTG/3Bio/ | 1603 | 4 |
| | 2^{nd} strand | /5Phos/TAGCCTGGGGCTTTGATGAT/3Bio/ | 1604 | |
| CHRNA3 rs1051730 MUT | 1^{st} strand | /5Phos/AAACACGACATCAAGTACAACTG/3Bio/ | 1605 | 9 |
| | 2^{nd} strand | /5Phos/TAGCCTGGGGCTTTGATGAT/3Bio/ | 1606 | |
| LOC1236 88 rs8034191 WT | 1^{st} strand | /5Phos/AGAAAGGCCCTGACAGATAGTAAC/3Bio/ | 1607 | 5 |
| | 2^{nd} strand | /5Phos/AACAGAAAACTTATACCACATTGGG/3Bio/ | 1608 | |
| LOC1236 88 rs8034191 MUT | 1^{st} strand | /5Phos/AGAAAGGCCCTGACAGATAGTAAC/3Bio/ | 1609 | 10 |
| | 2^{nd} strand | /5Phos/AACAGAAAACTTATACCACATTGGG/3Bio/ | 1610 | |
| ABCC1 rs212090 WT | 1^{st} strand | /5Phos/TCCCACCATGATTGATGTGGGG/3Bio/ | 1611 | 1 |
| | 2^{nd} strand | /5Phos/CAGTAATAACAGCATTGATTGTTCTTAC/3Bio/ | 1612 | |
| ABCC1 rs212090 MUT | 1^{st} strand | /5Phos/TCCCACCATGATTGATGTGGGG/3Bio/ | 1613 | 6 |
| | 2^{nd} strand | /5Phos/CAGTAATAACAGCATTGATTGTTCTTAC/3Bio/ | 1614 | |
| AURKA rs2273535 WT | 1^{st} strand | /5Phos/TTCCTTGTCAGAATCCATTACCTGTA/3Bio/ | 1615 | 3 |
| | 2^{nd} strand | /5Phos/TTGCTGAGTCACGAGAACACG/3Bio/ | 1616 | |
| AURKA rs2273535 MUT | 1^{st} strand | /5Phos/TTCCTTGTCAGAATCCATTACCTGTA/3Bio/ | 1617 | 8 |
| | 2^{nd} strand | /5Phos/TTGCTGAGTCACGAGAACACG/3Bio/ | 1618 | |
| CHEK2 rs1787996 1 WT | 1^{st} strand | /5Phos/TGCATACATAGAAGATCACAGTGG/3Bio/ | 1619 | 4 |
| | 2^{nd} strand | /5Phos/TGTAAGAGTTTTTAGGACCCACT/3Bio/ | 1620 | |
| CHEK2 rs1787996 1 MUT | 1^{st} strand | /5Phos/TGCATACATAGAAGATCACAGTGG/3Bio/ | 1621 | 9 |
| | 2^{nd} strand | /5Phos/TGTAAGAGTTTTTAGGACCCACT/3Bio/ | 1622 | |
| MPO rs2243828 WT | 1^{st} strand | /5Phos/GTGCTTGTCC CCAGGGGATA/3Bio/ | 1623 | 5 |
| | 2^{nd} strand | /5Phos/GGTATAGGCACACAATGGTGA/3Bio/ | 1624 | |
| MPO rs2243828 MUT | 1^{st} strand | /5Phos/GTGCTTGTCC CCAGGGGATA/3Bio/ | 1625 | 10 |

To test the probes for each mutation, probes from either the 1^{st} or 2^{nd} strand were chosen and tested against DNA extracted from a HL-60 cell line. DNA was extracted using the Gentra Puregene Cell Kit (Qiagen Cat# 158388) and 10 ng DNA was amplified using the MyTaq HS Master Mix (Bioline Cat# BIO-25045) and assay-specific PCR forward and revers primers, shown in Tables 44 and 45, respectively.

TABLE 44

PCR Forward Primers

| Assay | Forward Primer | SEQ ID NO: |
|---|---|---|
| MTHFR rs180113 | GTCATCCCTATTGGCAGGTTAC | 1626 |
| CDKN1A rs1801270 | CAGGGCCTTCCTTGTATCTC | 1627 |
| ABCB1 rs3842 | CCTCAGTCAAGTTCAGAGTCTTC | 1628 |
| CHRNA3 rs1051730 | TCCATGAACCTCAAGGACTATTG | 1629 |
| LOC123688 rs8034191 | GGTGATTGGTCCTCTGATTG | 1630 |
| ABCC1 rs212090 | GACTAACGGCTAACCTGGAC | 1631 |
| AURKA rs2273535 | TGAGCCTGGCCACTATTTAC | 1632 |
| CHEK2 rs17879961 | CTAGGAGAGCTGGTAATTTGGTC | 1633 |
| MPO rs2243828 | ACTACCAGCCCAAGATTTCTC | 1634 |

TABLE 45

PCR Reverse Primers

| Assay | Reverse Primer | SEQ ID NO: |
|---|---|---|
| MTHFR rs180113 | CTTCACAAAGCGGAAGAATGTG | 1635 |
| CDKN1A rs1801270 | TCGAAGTTCCATCGCTCAC | 1636 |
| ABCB1 rs3842 | AGCAAGGCAGTCAGTTACAG | 1637 |
| CHRNA3 rs1051730 | CGGATGTACAGCGAGTATGTG | 1638 |
| LOC123688 rs8034191 | CCCTGATTTCCACAAGTCC | 1639 |
| ABCC1 rs212090 | AGGCCATCTCCTTAATATTTACCC | 1640 |
| AURKA rs2273535 | CTTCCATTCTAGGCTACAGCTC | 1641 |
| CHEK2 rs17879961 | TCCATTGCCACTGTGATCTTC | 1642 |
| MPO rs2243828 | ATTCCTTGGGCTACCAGTTC | 1643 |

Following amplification, OLA was performed as described in Example 7 and the frequency of WT and Mutant alleles was determined. The results for each SNP are shown in Table 46.

TABLE 46

Frequency of WT and Mutant Alleles

| Assay | % Wild-type | % Mutant | Genotype |
|---|---|---|---|
| MTHFR rs180113 | 57.2 | 42.8 | Heterozygous |
| CDKN1A rs1801270 | 99.9 | 0.1 | Homozygous (WT) |
| ABCB1 rs3842 | 47.6 | 52.4 | Heterozygous |
| CHRNA3 rs1051730 | 99.9 | 0.1 | Homozygous (WT) |
| LOC123688 rs8034191 | 99.0 | 1.0 | Homozygous (WT) |
| ABCC1 rs212090 | 51.4 | 48.6 | Heterozygous |
| AURKA rs2273535 | 99.5 | 0.5 | Homozygous (WT) |
| CHEK2 rs17879961 | 99.2 | 0.8 | Homozygous (WT) |
| MPO rs2243828 | 46.4 | 53.6 | Heterozygous |

### Example 20. miRNA Detection Using an RNase Protection Assay

An RNase protection assay can be used for miRNA quantification. In the assay, a DNA/RNA chimeric probe is used that contains an oligonucleotide tag sequence that is complementary a capture oligonucleotide sequence and a miRNA complementary sequence. The oligonucleotide tag sequence can be a single stranded DNA (ssDNA) sequence included on the 5′ end of the chimeric probe and the miRNA complementary sequence can be a single stranded RNA (ssRNA) sequence at the 3′ end of the chimeric probe, along with a terminal 3′ biotin. The Sequence for miR-122 is shown in Table 47 and the chimeric probe is shown in Table 48.

Briefly, miRNA can be detected as follows:
1). Hybridize miRNA to the chimeric probe in a thermal cycler to form a hybridization product;
2). Block assay plate using one or more known blocking agent;
3). Add hybridization product with formamide to the blocked assay plate under conditions in which the oligonucleotide tag sequence of the hybridization product can hybridize to the capture oligonucleotides on the assay plate. Incubate at 37° C.;
4). Perform on-plate digestion of ssRNA with RNase A or RNase I (30° C. - 37° C.) to digest the following:
   a). ssRNA that has not been hybridized to the probe;
   b). Probe hybridized to the plate without the complementary miRNA; and
   c). RNA with as little as a single-base mismatch to the chimeric probe;
5). Add SULFO-TAG labeled streptavidin (SA-SULFO-TAG™) (Meso Scale Diagnostics, LLC, Rockville, MD) and incubate at room temperature;
6). Add Read Buffer B to assay plate and measure ECL signal generation with MSD Imager; and
7). Compare the results against a calibration curve for quantification.

The protocol above was used to detect the synthetic miR-122 miRNA. Results show that miR-122 was detected at 160 fM concentration, representing an improvement over results reported by Rissin et al., PLOS One (2017), which achieved detection of 500 fM of the same miRNA.

TABLE 47

miR-122 target sequence

| Target | Target Sequence | SEQ ID NO: |
|---|---|---|
| miR-122 | UGGAGUGUGACAAUGGUGUUUG | 1644 |

TABLE 48

miR-122 Chimeric Probe

| Target | Chimeric Probe | SEQ ID NO: |
|---|---|---|
| miR-122 | ACTGGTAACCCAGACATGATCGGT CAAACACCAUUGUCACACUCCA /3Bio/ 5′-Oligonucleotide tag complement (ssDNA)-miRNA complement (ssRNA)-3′ | 1645 |

### Example 21. ASO Detection Using Three Protocols (RNase Protection, OLA Using Taq DNA ligase, and OLA using T4 DNA ligase)

A model DNA ASO (GGC TAA ATC GCT CCA CCA AG; SEQ ID NO: 1646) was detected using three different protocols: an RNase protection assay, an OLA with Taq DNA ligase, and an OLA with T4 DNA ligase. Buffers used in all three protocols are as follows: blocking buffer contained Tris-HCl, a detergent, and cysteine; Hybridization Buffer 1 (“HB1”) contained Tris-HCl, a detergent, EDTA, salt, and formamide; Hybridization Buffer 2 (“HB2” used in all three protocols contained Tris-HCl, a detergent, and EDTA; dilution buffer contained Tris-HCl, a detergent, EDTA, and salt.

### A. RNase Protection Assay

A 10-point calibration curve was set up with a 4-fold dilution between calibrators and two blanks. The highest calibrator (Cal-1) concentration was 166 pM (~1 × 108 copies/ µL). Analyte or calibrator was originally diluted in water or in plasma to demonstrate a user-case scenario, where plasma is the most likely sample type. The starting sample size was 20 µL.

To the plasma samples, 2× RNAsecure (20 µL) was added and the mixture was heated to 60° C. for 10 min. A 5× master mix with chimeric probe was then added (10 µL) to the samples. Samples were hybridized to probes using the following protocol: 80° C. - 2 min; 65° C. - 5 min (then decrease 1° C. down to 50° C., each for 5 min); 37° C. - Hold.

The plate was blocked with blocking buffer for 30 min. @ 37° C. during hybridization. The hybridized product was diluted to 75 µL in Hybridization Buffer 2 and 30 µL added to 2 wells containing 20 µL Hybridization Buffer 1 (2:3 ratio HB1: HB2/sample). Hybridization to the plate was conducted for 1 hour @ 37° C.

RNase I was added to the plate and digestion was completed for 30 min. @ 37° C. Streptavidin A-SULFO-TAG (in dilution buffer) was added to the wells and incubated for 30 min. @ RT. Assay read buffer was added and the plate was read.

### B. OLA with Taq DNA Ligase

A 10-point calibration curve set up with a 4-fold dilution between calibrators and two blanks. The highest calibrator (Cal-1) concentration was 166 pM (~1 × 108 copies/ µL). Analyte or calibrator was originally diluted in water or in plasma to demonstrate a user-case scenario, where plasma is the most likely sample type. The starting sample size was 10 µL.

A 2× master mix was made that contained probes, Taq DNA ligase, and Taq ligase buffer. 25 µL was added to each sample (10 µL) and water (15 µL) prior to OLA. OLA cycling was performed as follows: 95° C. - 2 min; 95° C. - 30 sec; 37° C. - 5 min; 4° C. - hold.

The plate was blocked with blocking buffer for 30 min. @ 37° C. during cycling. The OLA product was diluted to 75 µL in Hybridization Buffer 2 and 30 µL added to 2 wells containing 20 µL Hybridization Buffer 1 (2:3 ratio HB1: HB2/sample). Hybridization to plate was conducted for 1 hour @ 37° C. Following hybridization, Streptavidin-SULFO-TAG (in dilution buffer) was added to wells and incubated for 30 min. @ RT. Assay read buffer was added and the plate was read.

### C. OLA With T4 DNA Ligase

A 10-point calibration curve set up with a 4-fold dilution between calibrators and two blanks. The highest calibrator (Cal-1) concentration was 166 pM (~1 × 108 copies/ µL). Analyte or calibrator was originally diluted in water or in plasma to demonstrate a user-case scenario, where plasma is the most likely sample type. The starting sample size was 20 µL.

A 2× master mix was made with probes and T4 DNA ligase buffer and was added (20 µL) to the samples. The samples were hybridized to the probes by ramping up to 95° C. for 2 min and cooling down to 65° C. at 50% ramp rate and then to 4° C. at a 3% ramp rate. T4 DNA Ligase was added and ligation at RT was completed for 30 min. The enzyme was inactivated by incubation at 65° C. for 10 min.

The plate was blocked with blocking buffer for 30 min. @ 37° C. during ligation/inactivation. The ligation product was diluted to 75 µL in Hybridization Buffer 2 and 30 µL added to 2 wells containing 20 µL Hybridization Buffer 1 (2:3 ratio HB1: HB2/sample). Hybridization to the plate was conducted for 1 hour @ 37° C. Following hybridization, Streptavidin-SULFO-TAG (in dilution buffer) was added to the wells and incubated for 30 min. @ RT. Assay read buffer was added and the plate was read.

### Example 22. ADA Detection Using Two Protocols (One-Step and Two-step)

Two methods were developed for detecting anti-drug antibody (ADA) against an antisense oligonucleotide. The same targeting probe and detecting probe can be used in either method. The targeting probe can include either a 12-mer oligonucleotide tag sequence GACATGATCGGT (SEQ ID NO: 1647) or a 24-mer oligonucleotide sequence ACTGGTAACCCAGACATGATCGGT (SEQ ID NO: 1648) and an antisense oligonucleotide sequence (ASO). If desired, a spacer sequence can be included between the oligonucleotide tag sequence and the ASO. The detecting probe includes a biotin label and the same antisense oligonucleotide sequence used with the targeting probe.

### A. “One Step”

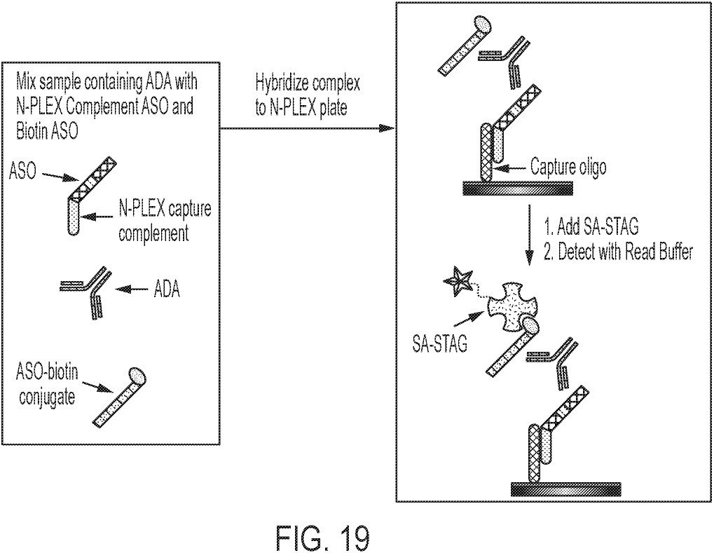

A schematic of the “one step” ADA detection method is shown in FIG. 19.

Briefly, at least about 250 nM targeting probe and at least about 250 nM detecting probe are combined with a sample that may include an anti-drug antibody (ADA) against the ASO on the targeting and detecting probes in a polypropylene plate to form a mixture. The mixture is incubated at room temperature with shaking (700 rpm) for 1 hour to allow the ADA, if present in the sample, to bind to the ASO on the targeting probe and the detecting probe.

A 96-well N-PLEX plate (MSD) on which a capture oligonucleotide that has a nucleotide sequence complementary to the oligonucleotide tag sequence is blocked with 50 µL of N-PLEX Blocker per well and incubated at 37° C. with shaking (700 rpm) for 30 minutes. The plate is then washed and 50 µL of the mixture from the polypropylene plate is transferred to each well of the N-PLEX plate and incubated at room temperature with shaking (700 rpm) for 1 hour to allow the oligonucleotide tags of the targeting probes in the mixture to hybridize to the capture oligonucleotides immobilized on the plate. The plate is washed to remove unbound species from the mixture and 50 µL of Streptavidin-SULFO-TAG in diluent is added to each well of the to the N-PLEX plate and incubated at RT with shaking (700 rpm) for 30 minutes to allow the Streptavidin-SULFO-TAG to bind to the Biotin moiety on any detection probe that is immobilized on the plate. The plate is washed, 150 µL of MSD Read Buffer is added to each well of the plate, and the presence of ADA is determined.

If the samples being tested for ADA also contain significant levels of the ASO drug (for example, if the sample is from a patient who received and has not yet cleared the drug), the ASO in the sample may be present as a complex with the ADA. If this occurs, the circulating ASO may block binding of the ADA to the targeting and detection probe and interfere with the detection of the ADA by the assay. In one aspect, the assay is tested to determine the sensitivity of the assay to the presence of ASO in a sample to determine at what level ASO present in the sample interferes with measurement of the ADA. In one aspect, if the assay is sensitive to interference from ASO in the sample, the method includes one or more steps to dissociate ASO in the sample from the ADA to prior to performing the assay. In one aspect, dissociation is achieved by exposing the sample to conditions that denature or otherwise destabilize the binding interaction between the ASO and ADA, but maintain the integrity of the ADA. In one aspect, dissociation is achieved by acidifying the sample. In one aspect, dissociation is achieved by making the sample basic. In one aspect, dissociation is achieved by heating the sample. In one aspect, dissociation is achieved by adding a denaturant to the sample. In one aspect, the sample is combined with the targeting and detection probe under conditions that are stabilizing to formation of ADA-ASO complexes, for example by neutralizing samples that had been acidified or made basic, by cooling samples that had been heated, or by diluting samples that had been treated with denaturants. In one aspect, the ASO is selectively degraded in the sample prior to testing. In one aspect, the ASO is selectively degraded based on the different nature of nucleic acids and proteins. In one aspect, the ASO is selectively degraded enzymatically, for example, using an enzyme capable of hydrolyzing phosphodiester bonds such as ribonucleases, deoxyribonucleases or other non-specific nucleases, phosphorylases or phosphomonoesterases. In one aspect, the ASO is degraded enzymatically, but probe degradation is prevented by inhibiting the nuclease prior to performing the assay.

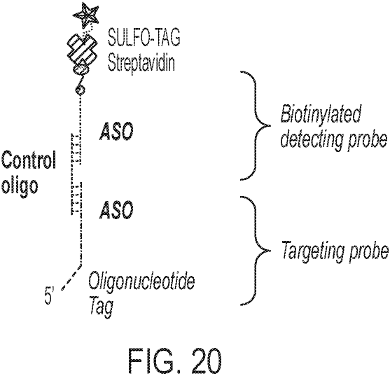

In one aspect, a positive control ADA is used that specifically binds to the ASO on the targeting and detecting probes to evaluate interference from circulating ASO, for example, by spiking different concentrations of ASO in samples containing positive control antibody. In another aspect, a positive control oligonucleotide that hybridizes to the ASO nucleotide sequence of the targeting probe and the detecting probe is used to optimize assay conditions (FIG. 20).

### B. Two Step

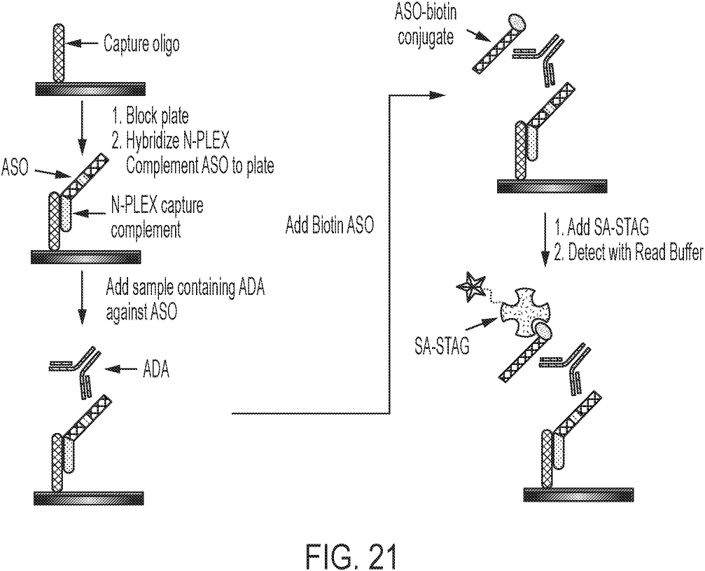

A schematic of the “two step” ADA detection method is shown in FIG. 21.

A 96-well N-PLEX plate (MSD) on which a capture oligonucleotide that has a nucleotide sequence complementary to the oligonucleotide tag sequence is blocked with 50 µL/well of N-PLEX Blocker and incubated at 37° C. with shaking (700 rpm) for 30 minutes. The plate is then washed and 50 µL of a mixture containing at least about 250 nM targeting probe in hybridization buffer is added to each well of the N-PLEX plate and incubate at 37° C. with shaking (700 rpm) for 1 hour to allow the oligonucleotide tags of the targeting probes to hybridize to the capture oligonucleotides hybridized on the plate. The plate is then washed and 50 µL of a sample that may include an anti-drug antibody (ADA) against the ASO on the targeting and at least about 250 nM detecting probe combined with a diluent is added to each well of the N-PLEX plate and incubated at room temperature with shaking (700 rpm) for 1 hour to allow the ADA, if present, to specifically bind to the ASO of the targeting probe immobilized on the plate. The plate is then washed and 50 µL of detection probe in diluent is added to the N-PLEX plate per well and incubated at room temperature with shaking (700 rpm) for 1 hour to allow the ASO of the detection probe to bind to the ADA immobilized on the plate. The plate is washed and 50 µL of Streptavidin-SULFO-TAG in diluent is added to each well of the N-PLEX plate and incubated at room temperature with shaking (700 rpm) for 30 minutes to allow the Streptavidin to bind to the biotin moiety of the detection probe immobilized on the plate. The plate is washed and 150 µL of MSD Read Buffer is added per well and the presence of ADA is determined.

If the samples being tested for ADA also contain significant levels of the ASO drug (for example, if the sample is from a patient who received and has not yet cleared the drug), the ASO in the sample may be present as a complex with the ADA. If this occurs, the circulating ASO may block binding of the ADA to the targeting and detection probe and interfere with the detection of the ADA by the assay. In one aspect, the assay is tested to determine the sensitivity of the assay to the presence of ASO in a sample to determine at what level ASO present in the sample interferes with measurement of the ADA. In one aspect, if the assay is sensitive to interference from ASO in the sample, the method includes one or more steps to dissociate ASO in the sample from the ADA to prior to performing the assay. In one aspect, dissociation is achieved by exposing the sample to conditions that are denaturing or otherwise destablizing the binding interaction between the ASO and ADA. In one aspect, dissociation is achieved by acidifying the sample. In one aspect, dissociation is achieved by making the sample basic. In one aspect, dissociation is achieved by heating the sample. In one aspect, dissociation is achieved by adding a denaturant to the sample. In one aspect, the sample is combined with the targeting and detection probe under conditions that are stabilizing to formation of ADA-ASO complexes, for example by neutralizing samples that had been acidified or made basic, by cooling samples that had been heated, or by diluting samples that had been treated with denaturants.

In one aspect, the ASO is selectively degraded in the sample prior to testing. In one aspect, the ASO is selectively degraded based on the different nature of nucleic acids and proteins. In one aspect, the ASO is selectively degraded enzymatically, for example, using an enzyme capable of hydrolyzing phosphodiester bonds such as ribonucleases, deoxyribonucleases or other non-specific nucleases, phosphorylases or phosphomonoesterases. In one aspect, the ASO is degraded enzymatically, but probe degradation is prevented by inhibiting the nuclease prior to performing the assay.

In one aspect, a positive control ADA is used that specifically binds to the ASO on the targeting and detecting probes to evaluate interference from circulating ASO, for example, by spiking different concentrations of ASO in samples containing positive control antibody. In another aspect, a positive control oligonucleotide that hybridizes to the ASO nucleotide sequence of the targeting probe and the detecting probe is used to optimize assay conditions (FIG. 20).

1. A set of two or more non-cross-reactive capture oligonucleotides (“capture set”), wherein the capture set is a subset of a parent set of capture oligonucleotides (“parent capture set”), wherein each capture oligonucleotide in the capture set comprises a) a nucleotide sequence comprising at least 20 consecutive nucleotides of a nucleotide sequence from the parent capture set or b) a nucleotide sequence having at least 95% identity to a nucleotide sequence from the parent capture set and wherein the parent capture set is selected from:
(a) parent capture set 1, comprising capture oligonucleotides having a nucleotide sequence selected from SEQ ID Nos: 1-64;
(b) parent capture set 2, comprising capture oligonucleotides having a nucleotide sequence selected from SEQ ID Nos: 65-122;
(c) parent capture set 3, comprising capture oligonucleotides having a nucleotide sequence selected from SEQ ID Nos: 123-186;
(d) parent capture set 4, comprising capture oligonucleotides having a nucleotide sequence selected from SEQ ID Nos: 187-250;
(e) parent capture set 5, comprising capture oligonucleotides having a nucleotide sequence selected from SEQ ID Nos: 251-308;
(f) parent capture set 6, comprising capture oligonucleotides having a nucleotide sequence selected from SEQ ID Nos: 309-372;
(g) parent capture set 7, comprising capture oligonucleotides having a nucleotide sequence selected from SEQ ID Nos: 373-436;
(h) parent capture set 8, comprising capture oligonucleotides having a nucleotide sequence selected from SEQ ID Nos: 437-494;
(i) parent capture set 9, comprising capture oligonucleotides having a nucleotide sequence selected from SEQ ID Nos: 495-558;
(j) parent capture set 10, comprising capture oligonucleotides having a nucleotide sequence selected from SEQ ID Nos: 559-662;
(k) parent capture set 11, comprising capture oligonucleotides having a nucleotide sequence selected from SEQ ID Nos: 623-680; and
1) parent capture set 12, comprising capture oligonucleotides having a nucleotide sequence selected from SEQ ID Nos: 681-744.

2. (canceled)

3. (canceled)

4. The capture set according to claim 1, wherein each capture oligonucleotide in the capture set is selected from:
(a) capture oligonucleotides having at least 20 consecutive nucleotides of a sequence selected from SEQ ID Nos: 1-64;
(b) capture oligonucleotides comprising a sequence having at least 95%, identity to a sequence selected from SEQ ID Nos: 1-64;
(c) capture oligonucleotides having at least 20 consecutive nucleotides of a sequence having at least 95%, identity to as sequence selected from SEQ ID Nos: 1-64;
(d) capture oligonucleotides comprising a sequence selected from SEQ ID Nos: 1-64; and
(e) capture oligonucleotides selected from any of (a)-(d).

5. The capture set according to claim 1, wherein each capture oligonucleotide in the set is selected from:
(a) capture oligonucleotides comprising a sequence having at least 20 consecutive nucleotides of a sequence selected from SEQ ID Nos: 1-10;
(b) capture oligonucleotides comprising a sequence having at least 95%, identity to a sequence selected from SEQ ID Nos: 1-10;
(c) capture oligonucleotides having at least 20 consecutive nucleotides of a sequence having at least 95% identity to as sequence selected from SEQ ID Nos: 1-10;
(d) capture oligonucleotides comprising a sequence selected from SEQ ID Nos: 1-10; and
(e) capture oligonucleotides selected from any of (a)-(d).

6. The capture set according to claim 1, wherein one or more capture oligonucleotides in the capture set comprise a reactive functional group.

7. The capture set according to claim 6, wherein the reactive functional group is attached to the capture oligonucleotide through a linker.

8. The capture set according to claim 6, wherein the reactive functional group comprises a thiol group.

9. The capture set according to claim 6, wherein the capture oligonucleotides are immobilized on a surface through the reactive functional group.

10. The capture set according to claim 9, wherein the surface comprises an electrode surface.

11. The capture set according to claim 10, wherein the electrode comprises a carbon-based electrode.

12. The capture set according to claim 11, wherein the electrode comprises a carbon ink electrode.

13. The capture set according to claim 1, wherein one or more capture oligonucleotides are at least 24 nucleotides long.

14. The capture set according to claim 1, wherein one or more capture oligonucleotides are at least 36 nucleotides long.

15. A kit comprising the capture set according to claim 1.

16. A kit comprising a capture set of 2 to 64 capture oligonucleotides according to claim 1.

17. A kit comprising a capture set of at least 10 capture oligonucleotides according to claim 1.

18. A set of two or more non-cross-reactive oligonucleotide tags (“tag set”), wherein the set of oligonucleotide tags is a subset of a parent set of oligonucleotide tags (“parent tag set”), wherein each oligonucleotide in the tag set comprises a) a nucleotide sequence comprising at least 20 consecutive nucleotides of a nucleotide sequencers from the parent tag set or b) a nucleotide sequence having at least 95% identity to a nucleotide sequence from the parent tag set and wherein the parent tag set is selected from:
(a) parent tag set 1, comprising oligonucleotide tags having a nucleotide sequence selected from SEQ ID Nos: 745-808;
(b) parent tag set 2, comprising oligonucleotide tags having a nucleotide sequence selected from SEQ ID Nos: 809-866;
(c) parent tag set 3, comprising oligonucleotide tags having a nucleotide sequence selected from SEQ ID Nos: 867-930;
(d) parent tag set 4, comprising oligonucleotide tags having a nucleotide sequence selected from SEQ ID Nos: 931-994;
(e) parent tag set 5, comprising oligonucleotide tags having a nucleotide sequence selected from SEQ ID Nos: 995-1052;
(f) parent tag set 6, comprising oligonucleotide tags having a nucleotide sequence selected from SEQ ID Nos: 1053-1116;
(g) parent tag set 7, comprising oligonucleotide tags having a nucleotide sequence selected from SEQ ID Nos: 1117-1180;
(h) parent tag set 8, comprising oligonucleotide tags having a nucleotide sequence selected from SEQ ID Nos: 1181-1238;
(i) parent tag set 9, comprising oligonucleotide tags having a nucleotide sequence selected from SEQ ID Nos: 1239-1302;
(j) parent tag set 10, comprising oligonucleotide tags having a nucleotide sequence selected from SEQ ID Nos: 1303-1366;
(k) parent tag set 11, comprising oligonucleotide tags having a nucleotide sequence selected from SEQ ID Nos: 1367-1424;
(1) parent tag set 12, comprising oligonucleotide tags having a nucleotide sequence selected from SEQ ID Nos: 1425-1488.

19-40. (canceled)

41. A method of immobilizing an oligonucleotide comprising a thiol group onto a carbon-based support surface, the method comprising:
(a) printing one or more liquid droplets comprising the oligonucleotide on the surface of the carbon-based electrode;
(b) allowing the liquid droplets to spread on the surface of the carbon-based electrode;
(c) drying the liquid droplets to form dried droplets;
(d) immobilizing the oligonucleotide to the carbon-based support surface through the thiol group; and
(e) washing the dried droplets with a wash solution comprising a thiol-containing compound to remove non-immobilized oligonucleotide.

42. (canceled)

43. (canceled)

44. The method according toclaim 41 comprising printing an array of liquid droplets comprising the capture oligonucleotides on a plurality of binding domains on the support surface.

45-65. (canceled)

66. A kit comprising:
(a) one or more support surfaces; and
(b) the capture set according to claim 1, wherein the capture set is immobilized on one or more support surfaces.

67. (canceled)

68. (canceled)

69. A kit comprising:
(a) one or more carbon-based electrodes having one or more surfaces; and
(b) the capture set according toclaim 1, wherein the capture set is immobilized on one or more support surfaces.

70-153. (canceled)

154. A method of identifying, detecting or quantifying a target analyte in a sample, the method comprising:
(a) contacting an array comprising:
(i) one or more carbon-based electrodes having one or more surfaces; and
(ii) the capture set according to claim 1 immobilized in one or more binding domains on one or more surfaces of the one or more carbon-based electrodes
with a composition comprising one or more target analytes, wherein the one or more target analytes are associated with a label and an oligonucleotide tag that is complementary to at least a portion of a capture oligonucleotide immobilized on the support surface and a label;
(b) incubating the composition of one or more target analytes under conditions in which the oligonucleotide tags hybridize to their corresponding complementary capture oligonucleotides to form a hybridization complex; and
(c) identifying, detecting or quantifying the target analyte based on the presence or absence of the label in an array location.

155-205. (canceled)
